(19) United States
(12) Patent Application Publication
(10) Pub. No.: US 2025/0099612 A1
(43) Pub. Date: Mar. 27, 2025

(54) VIRAL PARTICLES WITH MEMBRANE-BOUND HYALURONIDASE

(71) Applicant: Exuma Biotech Corp., West Palm Beach, FL (US)

(72) Inventors: Gregory Ian FROST, West Palm Beach, FL (US); Gregory Harold SCHREIBER, Palm Beach Gardens, FL (US)

(73) Assignee: Exuma Biotech Corp., West Palm Beach, FL (US)

(21) Appl. No.: 18/843,147

(22) PCT Filed: Mar. 1, 2023
(86) PCT No.: PCT/US23/63532

§ 371 Date: Aug. 30, 2024

Related U.S. Application Data
(60) Provisional application No. 63/362,741, filed on Apr. 8, 2022.
(60) Provisional application No. 63/315,509, filed on Mar. 1, 2022.
(60) Provisional application No. 63/315,523, filed on Mar. 1, 2022.

(51) Int. Cl. A61K 48/00; A61K 40/31; C12N 9/26

(52) U.S. Cl. CPC A61K 48/0041; A61K 40/31; C12N 9/2474; C12Y 302/01035; C07K 2319/03; C07K 2319/035; C12N 2740/16043; C12N 2740/16045

(57) ABSTRACT

Provided herein are methods, uses, compositions, and kits that include hyaluronidase polypeptides, including for example retroviral particles that include membrane bound hyaluronidase polypeptides, and novel, chimeric hyaluronidase polypeptides. Furthermore, provided herein are methods, uses, compositions, and kits that include hyaluronidase polypeptides, including for example retroviral particles that encode hyaluronidases and/or that include membrane bound hyaluronidases, which in some embodiments are novel, chimeric hyaluronidases provided herein. Accordingly, provided herein are replication incompetent recombinant retroviral particles (RIP), and methods using the same, that comprise a viral envelope, a nucleic acid encoding a packaging signal, Gag, and a membrane-bound hyaluronidase.

### CROSS-REFERENCE TO RELATED APPLICATIONS

This application claims priority to U.S. Provisional Application Ser. No. 63/315,509 filed on Mar. 1, 2022, U.S. Provisional Application Ser. No. 63/315,523 filed on Mar. 1, 2022, U.S. and Provisional Application Ser. No. 63/362,741 filed on Apr. 8, 2022. All of the applications cited in this paragraph are incorporated by reference herein in their entireties.

### SEQUENCE LISTING

This application hereby incorporates by reference the material of the electronic Sequencing Listing filed concurrently herewith. The materials in the electronic Sequence Listing is submitted as a text (.txt) file entitled “F1_008_WO_01_Sequence_Listing” created on Mar. 1, 2023, which has a file size of 513 KB, and is herein incorporated by reference in its entirety.

### FIELD OF INVENTION

This disclosure relates to the fields of polypeptides and viral particles that, in some embodiments, include hyaluronidase polypeptide on their surfaces and to methods for using the same.

### BACKGROUND OF THE DISCLOSURE

Hyaluronan (hyaluronic acid; HA) is a polypeptide that is found in the extracellular matrix of many cells, especially in soft connective tissues. HA also is found predominantly in skin, cartilage, and in Synovial fluid in mammals. Hyaluronan also is the main constituent of the vitreous of the eye. Hyaluronidases are enzymes that degrade hyaluron.

Hyaluronidases are enzymes that degrade hyaluronic acid. Six hyaluronidase genes have been identified in the human genome, all of which share homology, both within the paralog and across other vertabrates, including cows. Hyaluronidases facilitate the dispersion and absorption of biologics, including for example, viruses and cells through tissue. Furthermore, hyaluronidases can increase the lymphatic absorption profile of co-administered biologics when injected subcutaneously. When co-administered, however, hyaluronidases and the biologic may spread and disperse separately. There is a need for new hyaluronidase polypeptides that can harness the properties of existing hyaluronidases to create new enzymes with different properties for some or all of the functions of hyaluronidase. Furthermore, there is a need for new hyaluronidases that are better tailored for certain uses for which hyaluronidases are currently employed such as hyaluronidases that co-localize with the biologic. Thus, there is a need for therapeutics that can more effectively facilitate dispersion and absorption of biologics, including biologics that are co-administered with hyaluronidase polypeptides.

### SUMMARY

Provided herein are methods, uses, compositions, and kits that include hyaluronidase polypeptides, including for example retroviral particles that include membrane bound hyaluronidase polypeptides, and novel, chimeric hyaluronidase polypeptides. Provided herein are methods, uses, compositions, and kits that include hyaluronidase polypeptides, including for example retroviral particles that encode hyaluronidases and/or that include membrane bound hyaluronidases, which in some embodiments are novel, chimeric hyaluronidases provided herein. Accordingly, provided herein in one aspect is a replication incompetent recombinant retroviral particle (RIP), comprising a viral envelope, a nucleic acid encoding a packaging signal, Gag, and a membrane-bound hyaluronidase. Furthermore, provided herein are replication incompetent recombinant retroviral particle (RIP) formulations, comprising a plurality of RIPs, wherein each of the RIPs comprises a viral envelope, a nucleic acid encoding a packaging element, Gag, and a membrane-bound hyaluronidase polypeptide. Certain embodiments of such aspects, include novel chimeric hyaluronidase polypeptides provided herein. Furthermore, provided herein are delivery solutions, deliver reagents, formulations, pharmaceutical compositions, and methods that include that include such RIPs.

In one aspect, provided herein is a replication incompetent recombinant retroviral particle (RIP), comprising a viral envelope, a nucleic acid encoding a packaging signal, Gag and a membrane-bound hyaluronidase.

In another aspect, provided herein is a replication incompetent recombinant retroviral particle (RIP) formulation, comprising a plurality of RIPs, wherein each of the RIPs comprises a viral envelope, a nucleic acid encoding a packaging element, Gag, and a membrane-bound hyaluronidase polypeptide.

In some aspects, provided herein are isolated hyaluronidase polypeptides, and nucleic acids encoding the same. Such isolated hyaluronidase polypeptides include, for example, chimeric hyaluronidase polypeptides as provided herein.

In one aspect, provided herein is a polypeptide, comprising an amino acid sequence with at least 90% sequence identity to any one of SEQ ID NOs:544-1033.

Further details regarding aspects and embodiments of the present disclosure are provided throughout this patent application. Sections and section headers are for ease of reading and are not intended to limit combinations of disclosure, such as methods, compositions, and kits or functional elements therein across sections.

### BRIEF DESCRIPTION OF THE DRAWINGS

FIG. 1 shows an alignment of amino acids from human and bovine hyaluronidase precursor polypeptides and the stretch of amino acids corresponding to each of Regions 0, 1A, 1B, 2A, 2B, 3, 4, 5, 6, and 7.

FIG. 2 shows a table of the amino acid residue numbers from human and bovine hyaluronidase precursor polypeptides corresponding to each of Regions 0, 1A, 1B, 2A, 2B, 3, 4, 5, 6, and 7.

FIG. 3 shows a FACS plot of peripheral blood from an NSG-SGM3 CD34-humanized mouse 19 days after it was injected IP with GCAR-19GU RIPs.

FIG. 4 shows a graph of the number of B cells per μl blood in NSG-SGM3 CD34-humanized mice at various days after they were mock injected IP with PBS or injected IP with GCAR-19GU RIPs. The graph represents the average of 5 mice from each group.

FIG. 5 shows a FACS plot of peripheral blood from an NSG-MHC1/2-DKO PBMC-humanized mouse 19 days after it was injected IP with GCAR-19GU RIPs.

FIG. 6 shows flow cytometry data for CAR expression and markers of T and NK cells obtained from peripheral blood of mice 28 days post-dosing.

FIG. 7 shows a graph of the level (pg/ml) of select cytokines in the serum of mice 21 days after they were injected intravenously and/or subcutaneously with the test articles as indicated. The graph represents the averages from 5 mice in each group.

FIG. 8 shows a graph of CD19 CAR cell expansion following transduction with either F1-3-469 or F1-3-23. IL-2, IL-7, and anti-CD3 antibody were removed from the media on Day 7. “Fed” samples received Raji target cells every other day.

FIG. 9 shows graphs of the percentage of CD8+ cells that express PD-1, LAG3, TIM-3, or CD56 following transduction with the test articles as indicated.

FIG. 10 shows the expression of various surface markers by FACS contour plots of PBMCs transduced with F1-3-469GU after 14 days in culture.

### DEFINITIONS

As used herein, the term “about” refers to a value 10% less or 10% more than the disclosed value. For example, “about 1% sucrose” would include 0.9% to 1.1% sucrose.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

As used herein, the term cell “aggregate” means a cluster of cells that adhere to each other.

As used herein, the term “antibody” includes polyclonal and monoclonal antibodies, including intact antibodies and fragments of antibodies which retain specific binding to antigen. The antibody fragments can be, but are not limited to, fragment antigen binding (Fab) fragments, Fab′ fragments, F(ab′)₂ fragments, Fv fragments, Fab′-SH fragments, (Fab′)₂ Fv fragments, Fd fragments, recombinant IgG (rIgG) fragments, single-chain antibody fragments, including single-chain variable fragments (scFv), divalent scFv's, trivalent scFv's, a camelid, a VHH of a camelid, and single domain antibody fragments (e.g., sdAb, sdFv, nanobody). The term includes genetically engineered and/or otherwise modified forms of immunoglobulins, such as intrabodies, peptibodies, chimeric antibodies, single-chain antibodies, fully human antibodies, humanized antibodies, fusion proteins including an antigen-specific targeting region of an antibody and a non-antibody protein, heteroconjugate antibodies, multispecific, e.g., bispecific, antibodies, diabodies, triabodies, and tetrabodies, tandem di-scFv's, and tandem tri-scFv's. Unless otherwise stated, the term “antibody” should be understood to include functional antibody fragments thereof. The term also includes intact or full-length antibodies, including antibodies of any class or sub-class, including IgG and sub-classes thereof, IgM, IgE, IgA, and IgD.

As used herein, the term “antibody fragment” includes a portion of an intact antibody, for example, the antigen binding or variable region of an intact antibody. Examples of antibody fragments include Fab, Fab′, F(ab′)₂, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fe” fragment, a designation reflecting the ability to crystallize readily. Pepsin treatment yields an F(ab′)₂ fragment that has two antigen combining sites and is still capable of cross-linking antigen.

As used herein, “antibody mimetic” refers to an organic compound that specifically binds a target sequence and has a structure distinct from a naturally-occurring antibody. Antibody mimetics may comprise a protein, a nucleic acid, or a small molecule, and a skilled artisan can understand when each type is relevant. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Antibody mimetics include, but are not limited to, an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain.

As used herein, an “approved biologic” is a macromolecule that meets the requirements of a biologic provided by a government regulatory agency such as, but not limited to, the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan and has been approved by such regulatory agency either as a stand-alone biologic, or as part of a combination product or method.

As used herein, the term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, reference to a “cell surface expression system” or “cell surface display system” refers to the display or expression of a protein or portion thereof on the surface of a cell. Typically, a cell is generated that expresses proteins of interest fused to a cell-surface protein. For example, a protein is expressed as a fusion protein with a transmembrane domain.

A pseudotyping element as used herein can include a “binding polypeptide” that includes one or more polypeptides, typically glycoproteins, that identify and bind the target host cell, and one or more “fusogenic polypeptides” that mediate fusion of the retroviral and target host cell membranes, thereby allowing a retroviral genome to enter the target host cell. The “binding polypeptide” as used herein, can also be referred to as a “T cell and/or NK cell binding polypeptide” or a “target engagement element,” and the “fusogenic polypeptide” can also be referred to as a “fusogenic element”.

As used herein, the term “chimeric antigen receptor” or “CAR” or “CARs” refers to engineered receptors, which graft an antigen specificity onto cells, for example T cells, NK cells, macrophages, and stem cells. The CARs of the invention include at least one antigen-specific targeting region (ASTR), a transmembrane domain (TM), and an intracellular activating domain (IAD) and can include a stalk, and one or more co-stimulatory domains (CSDs). In another embodiment, the CAR is a bispecific CAR, which is specific to two different antigens or epitopes. After the ASTR binds specifically to a target antigen, the IAD activates intracellular signaling. For example, the IAD can redirect T cell specificity and reactivity toward a selected target in a non-MHC-restricted manner, exploiting the antigen-binding properties of antibodies. The non-MHC-restricted antigen recognition gives T cells expressing the CAR the ability to recognize an antigen independent of antigen processing, thus bypassing a major mechanism of tumor escape. Moreover, when expressed in T cells, CARs advantageously do not dimerize with endogenous T cell receptor (TCR) alpha and beta chains.

As used herein, “complementarity-determining region” or “CDR” refers to the three hypervariable regions in each variable chain of immunoglobulins and T cell receptors that interrupt the four “framework” regions of the chains. The CDRs are primarily responsible for the specificity of binding. The CDRs of each immunoglobulin chain are referred to as CDR1, CDR2, and CDR3, numbered sequentially starting from the N-terminus, and are also typically identified by the chain in which the particular CDR is located. Thus, HCDR3 is located in the variable domain of the heavy chain of the antibody in which it is found, whereas a LCDR1 is the CDR1 from the variable domain of the light chain of the antibody in which it is found. The sequences of the framework regions of different light or heavy chains are relatively conserved within a species. The framework region of an antibody, that is the combined framework regions of the constituent light and heavy chains, serves to position and align the CDRs in three dimensional space. The amino acid sequences of the CDRs and framework regions can be determined using various well-known definitions in the art, e.g., Kabat, Chothia, international ImMunoGeneTics database (IMGT), and AbM (see, e.g., Johnson and Wu, Nucleic Acids Res. 2000 Jan. 1; 28(1): 214-218 and Johnson et al., Nucleic Acids Res., 29:205-206 (2001); Chothia & Lesk, (1987) J. Mol. Biol. 196, 901-917; Chothia et al. (1989) Nature 342, 877-883; Chothia et al. (1992) J. Mol. Biol. 227, 799-817; Al-Lazikam et al., J. Mol. Biol. 1997, 273(4)). Unless otherwise indicated, CDRs herein are determined using “Fab Analysis” on the World Wide Web at vbase2.org (Retter et al., Nucleic Acids Res., 33:D671-D674 and Mollova et al., BMC Systems Bio., S1, p30)).

As used herein, the term “constitutive T cell or NK cell promoter” refers to a promoter which, when operably linked with a polynucleotide that encodes or specifies a gene product, causes the gene product to be produced in a cell under most or all physiological conditions of the cell.

As used herein, the term “construct” refers to an isolated polypeptide or an isolated polynucleotide encoding a polypeptide. A polynucleotide construct can encode a polypeptide, for example, a lymphoproliferative element. A skilled artisan will understand whether a construct refers to an isolated polynucleotide or an isolated polypeptide depending on the context.

As used herein, a “cytotoxic cell” includes CD8⁺ T cells, natural-killer (NK) cells, NK-T cells, γδ T cells, a subpopulation of CD4⁺ cells, and neutrophils, which are cells capable of mediating cytotoxicity responses.

As used herein, a “domain” is a region of a polypeptide or polynucleotide with a functional and/or structural property.

As used herein, “double stranded RNA” or “dsRNA” or “RNA duplex” refers to RNA molecules that are comprised of two strands. Double-stranded molecules include those comprised of two RNA strands that hybridize to form the duplex RNA structure or a single RNA strand that doubles back on itself to form a duplex structure. Most, but not necessarily all of the bases in the duplex regions are base-paired. The duplex region comprises a sequence complementary to a target RNA. The sequence complementary to a target RNA is an antisense sequence, and is frequently from 18 to 29, from 19 to 29, from 19 to 21, or from 25 to 28 nucleotides long, or in some embodiments between 18, 19, 20, 21, 22, 23, 24, 25 on the low end and 21, 22, 23, 24, 25, 26, 27, 28 29, or 30 on the high end, where a given range always has a low end lower than a high end. Such structures typically include a 5′ stem, a loop, and a 3′ stem connected by a loop which is contiguous with each stem and which is not part of the duplex. The loop comprises, in certain embodiments, at least 3, 4, 5, 6, 7, 8, 9, or 10 nucleotides. In other embodiments the loop comprises from 2 to 40, from 3 to 40, from 3 to 21, or from 19 to 21 nucleotides, or in some embodiments between 2, 3, 4, 5, 6, 7, 8, 9, 10, 11, 12, 13, 14, 15, 16, 17, 18, 19, or 20 on the low end and 10, 11, 12, 13, 14, 15, 16, 17, 18, 19, 20, 25, 30, 35, or 40 on the high end, where a given range always has a low end lower than a high end.

As used herein, the term “element” includes polypeptides, including fusions of polypeptides, regions of polypeptides, and functional mutants or fragments thereof and polynucleotides, including microRNAs and shRNAs, and functional mutants or fragments thereof.

As used herein, the term “evolution” or “evolving” refers to using one or more methods of mutagenesis to generate a different polynucleotide encoding a different polypeptide, which is itself an improved biological molecule and/or contributes to the generation of another improved biological molecule. “Physiological” or “normal” or “normal physiological” conditions are conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions, that would be considered within a normal range at the site of administration, or at the tissue or organ at the site of action, to a subject.

A “framework” of a miRNA consists of “5′ microRNA flanking sequence” and/or “3′ microRNA flanking sequence” surrounding a miRNA and, in some cases, a loop sequence that separates the stems of a stem-loop structure in a miRNA. In some examples, the “framework” is derived from naturally occurring miRNAs, such as, for example, miR-155. The terms “5′ microRNA flanking sequence” and “5′ arm” are used interchangeably herein. The terms “3′ microRNA flanking sequence” and “3′ arm” are used interchangeably herein.

As used herein, “hyaluronidase” refers to an enzyme that degrades hyaluronic acid.

As used herein “sequence identity”, has a meaning recognized in the art. Thus, by a first polypeptide sequence having an amino acid sequence at least, for example, 95% “identical” to a second polypeptide sequence having an amino acid sequence disclosed herein, it is intended that the amino acid sequence of the first polypeptide is identical to the amino acid sequence of the second polypeptide except that the first polypeptide sequence may include up to five amino acid alterations per each 100 amino acids of the second polypeptide sequence. In other words, to obtain a first polypeptide having an amino acid sequence at least 95% identical to a second polypeptide sequence, up to 5% of the amino acid residues in the first polypeptide sequence may be insertions, deletions, or substitutions when aligned with the amino acid sequence of the second polypeptide. These alterations as compared to the second sequence may occur at the amino- or carboxy-terminal positions of the second amino acid sequence or anywhere between those terminal positions, interspersed either individually among residues in the second sequence or in one or more contiguous groups within the second sequence. The identity of two or more sequences (e.g., amino acid sequences) can be compared to one another, or to published sequences, using the Basic Local Alignment Search Tool or “BLAST” algorithm; described in Johnson M, et al., (2008) NCBI BLAST: a better web interface. Nucleic Acids Res. 36:W5-W9 (incorporated herein by reference in its entirety). Similarly, identity can be determined between two nucleotide sequences in the same manner. Thus, to obtain a first nucleotide sequence (e.g., RNA or DNA sequence, such as a cDNA sequence) that is at least 95% identical to a second nucleotide sequence, up to 5% of the nucleotide residues in the first sequence may be insertions, deletions, or substitutions when aligned with nucleotide sequence of the second nucleotide sequence.

As used herein, the term “idiotype” refers to the segment of an antibody or an antibody mimetic that determines its specificity for antigen, for example, a structure of a variable region of an antibody, a T cell receptor, or an antibody mimetic that is a shared characteristic between a group of antibodies, T-cell receptors, or antibody mimetics based upon the antigen binding specificity and therefore structure of their variable regions. The idiotype of an antibody typically includes the variable region, e.g., the CDRs and framework regions. For antibodies, the idiotype is located in the Fab region. For antibodies formed with multiple chains, e.g., heavy chains and light chains, expression of the idiotype usually requires participation of the variable regions of both heavy and light chains that form the antigen-combining site. For antibodies formed with single chains, e.g., scFv, expression of the idiotype usually requires participation of the variable regions of one polypeptide that forms the antigen-combining site. For antibody mimetics, the idiotype varies depending on the type of antibody mimetic, but includes the region necessary for binding the cognate antigen.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

As used interchangeably herein, the terms “individual”, “subject”, “host”, and “patient” refer to a mammal, including, but not limited to, humans, murines (e.g., rats, mice), lagomorphs (e.g., rabbits), non-human primates, humans, canines, felines, ungulates (e.g., equines, bovines, ovines, porcines, caprines), etc.

As used herein, the terms “inducible promoter” or “activatable promoter” refer to promoters that when operably linked with a polynucleotide that encodes or specifies a gene product, cause the gene product to be produced in a cell substantially only when a promoter-specific inducer is present in the cell. Inducible promoters have no, or a low level, of basal transcription activity but the transcription activity increases, sometimes greatly, in the presence of an inducing signal.

As used herein, the term “insulator” refers to a cis-regulatory element that mediates intra- and inter-chromosomal interactions and can block interactions between enhancers and promoters. Typically, insulators are between 200 and 2000 base pairs in length and contain clustered binding sites for sequence specific DNA-binding proteins.

As used herein, the term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. The term “linker” when used in reference to a multiplex inhibitory RNA molecule refers to a connecting means that joins two inhibitory RNA molecules.

As used herein, “lymphodepletion” involves methods that reduce the number of lymphocytes in a subject, for example by administration of a lymphodepletion agent. Lymphodepletion can also be attained by partial body or whole body fractioned radiation therapy. A lymphodepletion agent can be a chemical compound or composition capable of decreasing the number of functional lymphocytes in a mammal when administered to the mammal. One example of such an agent is one or more chemotherapeutic agents. Such agents and dosages are known, and can be selected by a treating physician depending on the subject to be treated. Examples of lymphodepletion agents include, but are not limited to, fludarabine, cyclophosphamide, cladribine, denileukin diftitox, alemtuzumab or combinations thereof.

As used herein, the term “microenvironment” means any portion or region of a tissue or body that has constant or temporal, physical, or chemical differences from other regions of the tissue or regions of the body. For example, a “tumor microenvironment” as used herein refers to the environment in which a tumor exists, which is the non-cellular area within the tumor and the area directly outside the tumorous tissue but does not pertain to the intracellular compartment of the cancer cell itself. The tumor microenvironment can refer to any and all conditions of the tumor milieu including conditions that create a structural and or functional environment for the malignant process to survive and/or expand and/or spread. For example, the tumor microenvironment can include alterations in conditions such as, but not limited to, pressure, temperature, pH, ionic strength, osmotic pressure, osmolality, oxidative stress, concentration of one or more solutes, concentration of electrolytes, concentration of glucose, concentration of hyaluronan, concentration of lactic acid or lactate, concentration of albumin, levels of adenosine, levels of R-2-hydroxyglutarate, concentration of pyruvate, concentration of oxygen, and/or presence of oxidants, reductants, or co-factors, as well as other conditions a skilled artisan will understand.

The term “microRNA flanking sequence” as used herein refers to nucleotide sequences including microRNA processing elements. MicroRNA processing elements are the minimal nucleic acid sequences which contribute to the production of mature microRNA from precursor microRNA. Often these elements are located within a 40 nucleotide sequence that flanks a microRNA stem-loop structure. In some instances, the microRNA processing elements are found within a stretch of nucleotide sequences of between 5 and 4,000 nucleotides in length that flank a microRNA stem-loop structure.

As used herein, “MOI”, refers to Multiplicity of Infection ratio where the MOI is equal to the ratio of the number of virus particles used for infection per number of cells. Functional titering of the number of virus particles can be performed using FACS and reporter expression, as non-limiting examples.

As used herein, “naturally occurring” VH and VL domains refer to VH and VL domains that have been isolated from a host without further molecular evolution to change their affinities when generated in an scFv format under specific conditions such as those disclosed in U.S. Pat. No. 8,709,755 B2 and application WO/2016/033331A1.

As used herein, “NK cell” includes lymphocytes that express CD56 on their surface (CD56+ lymphocytes). NKT cells, which are CD3+, CD56+, and either CD4+ or CD8+, are considered a type of NK cells herein. NKT cells may also be positive for CD57.

“Peripheral blood mononuclear cells” (PBMCs) include peripheral blood cells having a round nucleus and include lymphocytes (e.g., T cells, NK cells, and B cells) and monocytes. Some blood cell types that are not PBMCs include red blood cells, platelets and granulocytes (i.e., neutrophils, eosinophils, and basophils).

As used interchangeably herein, the terms “polynucleotide” and “nucleic acid” refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, a “polypeptide” is a single chain of amino acid residues linked by peptide bonds. A polypeptide does not fold into a fixed structure nor does it have any posttranslational modification. A “protein” is a polypeptide that folds into a fixed structure. “Polypeptides” and “proteins” are used interchangeably herein.

A “polypeptide” as used herein can include part of or an entire protein molecule as well as any posttranslational or other modifications.

As used herein, a polypeptide may be “purified” to remove contaminant components of a polypeptide's natural environment, e.g., materials that would interfere with diagnostic or therapeutic uses for the polypeptide such as, for example, enzymes, hormones, and other proteinaceous or nonproteinaceous solutes. A polypeptide can be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain.

As used herein, the term “recognize” refers to the ability of one molecule to bind to another molecule, for example, the ability of a receptor to bind its ligand or the ability of an antibody to bind its target.

As used herein, the term “region” is any segment of a polypeptide or polynucleotide.

A “resting” lymphocyte, such as for example, a resting T cell, is a lymphocyte in the G0 stage of the cell cycle that does not express activation markers such as Ki-67. Resting lymphocytes can include naïve T cells that have never encountered specific antigen and memory T cells that have been altered by a previous encounter with an antigen. A “resting” lymphocyte can also be referred to as a “quiescent” lymphocyte.

RNA interference (RNAi) is a biological process in which RNA molecules inhibit gene expression or translation by neutralizing targeted RNA molecules. The RNA target may be mRNA, or it may be any other RNA susceptible to functional inhibition by RNAi. As used herein, an “inhibitory RNA molecule” refers to an RNA molecule whose presence within a cell results in RNAi and leads to reduced expression of a transcript to which the inhibitory RNA molecule is targeted. An inhibitory RNA molecule as used herein has a 5′ stem and a 3′ stem that is capable of forming an RNA duplex. The inhibitory RNA molecule can be, for example, a miRNA (either endogenous or artificial) or a shRNA, a precursor of a miRNA (i.e., a Pri-miRNA or Pre-miRNA) or shRNA, or a dsRNA that is either transcribed or introduced directly as an isolated nucleic acid, to a cell or subject.

As used herein, a “recombinant retrovirus” refers to a non-replicable, or “replication incompetent”, retrovirus unless it is explicitly noted as a replicable retrovirus. The terms “recombinant retrovirus” and “recombinant retroviral particle” are used interchangeably herein. Such retrovirus/retroviral particle can be any type of retroviral particle including, for example, gamma retrovirus, and in illustrative embodiments, lentivirus. As is known, such retroviral particles, for example lentiviral particles, typically are formed in packaging cells by transfecting the packing cells with plasmids that include packaging components such as Gag, Pol and Rev, an envelope or pseudotyping plasmid that encodes a pseudotyping element, and a transfer, genomic, or retroviral (e.g., lentiviral) expression vector, which is typically a plasmid on which a gene(s) or other coding sequence of interest is encoded. Accordingly, a retroviral (e.g., lentiviral) expression vector includes sequences (e.g., a 5′ LTR and a 3′ LTR flanking e.g., a psi packaging element and a target heterologous coding sequence) that promote expression and packaging after transfection into a cell. The terms “lentivirus” and “lentiviral particle” are used interchangeably herein.

As used herein, reference to a “cell surface expression system” or “cell surface display system” refers to the display or expression of a protein or portion thereof on the surface of a cell. Typically, a cell is generated that expresses proteins of interest fused to a cell-surface protein. For example, a protein is expressed as a fusion protein with a transmembrane domain.

As used herein, the term “element” includes polypeptides, including fusions of polypeptides, regions of polypeptides, and functional mutants or fragments thereof and polynucleotides, including microRNAs and shRNAs, and functional mutants or fragments thereof.

As used interchangeably herein, the terms “single-chain Fv,” “scFv,” or “sFv” antibody fragments include the VH and VL domains of antibody, wherein these domains are present in a single polypeptide chain. In some embodiments, the Fv polypeptide further includes a polypeptide linker or spacer between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used interchangeably herein, the terms “stalk” or “stalk domain” refer to a flexible polypeptide connector region providing structural flexibility and spacing to flanking polypeptide regions and can consist of natural or synthetic polypeptides. A stalk can be derived from a hinge or hinge region of an immunoglobulin (e.g., IgG1) that is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The stalk may be of natural occurrence or non-natural occurrence, including but not limited to an altered hinge region, as disclosed in U.S. Pat. No. 5,677,425. The stalk can include a complete hinge region derived from an antibody of any class or subclass. The stalk can also include regions derived from CD8, CD28, or other receptors that provide a similar function in providing flexibility and spacing to flanking regions.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, etc.).

As used herein, “TaNK cell” includes lymphocytes that express CD3, CD8, and CD56 (CD3+/CD8+/CD56+ lymphocytes).

As used herein, “T cell” includes all types of immune cells expressing CD3 including T-helper cells (CD4+ cells), cytotoxic T cells (CD8+ cells), T-regulatory cells (Treg) and gamma-delta T cells. NKT cells, which are CD3+, CD56+, and either CD4+ or CD8+, are considered a type of T cells herein. Surface expression of CD3 can be transiently decreased or eliminated in T cells, as has been observed with some of the methods for modifying T cells disclosed herein. Such modified CD4+ or CD8+ lymphocytes that have transiently decreased/absent CD3 surface expression, are still considered T cells in this disclosure. Reference to a “CD” or cluster of differentiation marker, such as CD3+, CD4+, CD8+, CD56+ herein, relates to surface expression of such polypeptide. It will be understood that surface expression is a continuum between positive and negative, and can be assessed by FACS analysis, where cells are determined to be positive or negative based on user cutoffs known in the art. A low and intermediate expression of a surface marker determined by FACS analysis, such as CD3lo or CD3int, are considered surface marker negative (e.g., CD3−) herein.

As used herein, a “therapeutic antibody” or “therapeutic antibody mimetic” is an antibody or an antibody mimetic that has been demonstrated using an in vivo assay, for example, in humans, to have therapeutic activity.

As used herein, the terms “therapeutically effective amount” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

As used herein, a “transduced cell” or a “stably transfected cell” is a cell that contains an exogenous nucleic acid(s) that is integrated into the genome of the cell. As used herein, a “genetically modified cell” is a cell that contains an exogenous nucleic acid(s) regardless of whether the exogenous nucleic acid(s) is integrated into the genome of the cell, and regardless of the method used to introduce the exogenous nucleic acid(s) into the cell. Exogenous nucleic acid(s) inside a cell that are not integrated into the genome of the cell can be referred to as “extrachromosomal” herein. As used herein, a “modified cell” is a cell that is associated with a recombinant nucleic acid vector (also called a “polynucleotide vector” or a “gene vector” herein), which in illustrative embodiments is a replication incompetent recombinant retroviral particle (also called a “RIR retroviral particle” or a “RIP” herein), that contains an exogenous nucleic acid, or a cell that has been genetically modified by an exogenous nucleic acid. Typically, in compositions and methods that include a replication incompetent recombinant retroviral particle, a modified cell associates with a replication incompetent recombinant retroviral particle through interactions between proteins on the surface of the cell and proteins on the surface of the replication incompetent recombinant retroviral particle, including pseudotyping elements and/or T cell activation elements. In compositions and methods that include transfection of nucleic acid inside a lipid-based reagent, such as a liposomal reagent, the lipid-based reagent containing nucleic acid, which is a type of recombinant nucleic acid vector, associates with the lipid bilayer of the modified cell before fusing or being internalized by the modified cell. Similarly, in compositions and methods that include chemical-based transfection of nucleic acid, such as polyethylenimine (PEI) or calcium phosphate-based transfection, the nucleic acid is typically associated with a positively charged transfection reagent to form the recombinant nucleic acid vector that associates with the negatively charged membrane of the modified cell before the complex is internalized by the modified cell. Other means or methods of stably transfecting or genetically modifying cells include electroporation, ballistic delivery, and microinjection.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “miRNA precursor” refers to an RNA molecule of any length which can be enzymatically processed into an miRNA, such as a primary RNA transcript, a pri-miRNA, or a pre-miRNA.

As used herein, the term “syncytium-inducing polypeptide” refers to a membrane polypeptide or a portion thereof that causes cell fusion, with such cell fusion leading to the formation of syncytia. Syncytium-inducing polypeptides are a type of fusogenic envelope protein as disclosed herein. Syncytium-inducing polypeptides as used herein encompass those proteins naturally produced by viruses, particularly the so-called fusogenic membrane proteins (FMPs) and fusogenic membrane glycoproteins (FMGs), that mediate virus-cell fusion, as well as cell-cell fusion of infected cells. Syncytium-inducing polypeptides as used herein further encompass non-viral polypeptides known to mediate cell-cell fusion events in vivo. A “viral fusogenic membrane glycoprotein” is a virally-derived fusogenic membrane protein that, in nature, mediates membrane fusion of a virus to its host target cell. A syncytium-inducing polypeptide (or portion thereof) or fusogenic membrane glycoprotein (or portion thereof), as used herein, has the ability, when in isolation from a virus, to mediate or induce fusion between a cell expressing the fusogenic membrane glycoprotein and a cell expressing a receptor for the fusogenic membrane glycoprotein. Examples of fusogenic membrane proteins include, but are not limited to fertilin b. The viral fusogenic membrane glycoprotein subset of the fusogenic membrane proteins includes, but is not limited to: type G glycoproteins in Rabies, Mokola, vesicular stomatitis and Toga viruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus, canine distemper virus, Newcastle disease virus, human parainfluenza virus 3, simian virus 41, Sendai virus and human respiratory syncytial virus; gH of human herpes virus 1 and simian varicella virus, with the chaperone protein gL; human, bovine and cercopithicine herpesvirus gB; envelope glycoproteins of Friend murine leukemia virus and Mason Pfizer monkey virus; influenza haemagglutinin; G protein of Vesicular Stomatitis Virus; mumps virus hemagglutinin neuraminidase, and glycoproteins FI and F2; and membrane glycoproteins from Venezuelan equine encephalomyelitis.

It is recognized herein that some syncytium-inducing polypeptides function alone, while others require more than one different polypeptide to have fusion-promoting activity. As used herein then, the singular term “syncytium-inducing polypeptide” is meant to encompass single fusion-promoting polypeptides as well as each of the polypeptides required for promoting fusion when there is a requirement for more than one.

As used herein, the term “transducing units” refers to the number of viral particles in a solution that are capable of transducing a target cell, and can be calculated by transducing any target cell, for example, Jurkat cells, and measuring the expression of a transgene in the target cells, as determined, for example, by serial dilution and analysis of transgene expression by qPCR for the lentiviral genome using the Lenti-X™ qRT-PCR Titration Kit (Takara, #631235).”

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within embodiments herein, subject to any specifically excluded limit in the stated range. Where the stated range includes one or both of the limits, ranges excluding either or both of those included limits are also included in the embodiments herein. When multiple low and multiple high values for ranges are given that overlap, a skilled artisan will recognize that a selected range will include a low value that is less than the high value. All headings in this specification are for the convenience of the reader and are not limiting.

Unless defined otherwise, all technical and scientific terms used herein have the same meaning as commonly understood by one of ordinary skill in the art to which the aspects and embodiments provided herein belong. Although any methods and materials similar or equivalent to those described herein can also be used in the practice or testing of any aspects or embodiments provided herein, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless specifically stated or otherwise obvious from context, as used herein, the term “or” is understood to be inclusive. The term “and/or” as used in a phrase such as “A and/or B” herein includes each of the following: A and B; A or B; A (alone); and B (alone). Similarly, the term “and/or” as used in a phrase such as “A, B, and/or C” includes each of the following: A, B, and C; A, B, or C; A or B; A or C; B or C; A and B; A and C; B and C; A (alone); B (alone); and C (alone). This logic extends to any number of items in a list that are connected with the term “and/or”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### DETAILED DESCRIPTION

The present disclosure overcomes prior art challenges by providing improved compositions that have many uses, including RIPs encoding hyaluronidase polypeptides and/or having membrane-bound hyaluronidase polypeptides, novel hyaluronidase polypeptides, including chimeric hyaluronidase polypeptides, and combinations thereof. Such compositions can be included in RIP formulations, research reagents, delivery reagents, and pharmaceutical compositions, which can be useful for any route of administration to improve the penetration of RIPs or other molecules, such as other biologics, delivered with the chimeric hyaluronidase polypeptides. In certain embodiments, such compositions are effective, and optionally adapted for any route of administration, and in certain illustrative embodiments are effective for and/or adapted for perilymphatic, intraperitoneal, intranodal or subcutaneous administration. Such compositions (e.g. RIP formulations) have utility in the treatment of virtually any disorder, for example those that can be treated by using virtually any type of small molecule therapeutic, or biologic such as a cell or gene therapy, in illustrative embodiments, for the treatment of hyperproliferative cell disorders such as cancer. Chimeric hyaluronidase polypeptides disclosed herein, in some embodiments include various combinations of regions from different hyaluronidase polypeptides.

### Recombinant Retroviral Particles

Retroviral particles and recombinant retroviral particles, that in illustrative embodiments include membrane-bound hyaluronidase polypeptide, are illustrative aspects provided herein, and are utilized in methods and compositions provided herein. Replication incompetent retroviral particles and recombinant, replication incompetent retroviral particles provided herein, each of which in illustrative embodiments include membrane-bound hyaluronidase, can be used for example, to modify cells, as non-limiting examples human cells, primary cells, T cells and/or NK cells to make genetically modified and/or transduced cells, human cells, primary cells, T cells and/or NK cells. Such modifying can occur in vivo, ex vivo, or in vitro. The recombinant retroviral particles are themselves aspects of the present disclosure. Typically, the recombinant retroviral particles included in aspects provided herein, are replication incompetent, meaning that a recombinant retroviral particle cannot replicate once it leaves the packaging cell. In fact, unless indicated otherwise herein, retroviral particles are replication incompetent, and if such retroviral particles include nucleic acids in their genome that are not native to the retrovirus, they are “recombinant retroviral particles.” In illustrative embodiments, the recombinant retroviral particles are lentiviral particles. It is noteworthy that in some embodiments, where the disclosure recites RIPs or recombinant retroviral particles, embodiments are contemplated wherein the retroviral particle is not recombinant, thus, for example, it has nucleic acid sequences that are found in a wild type retrovirus.

Provided herein in some aspects are replication incompetent recombinant retroviral particles that in illustrative embodiments include membrane-bound hyaluronidase polypeptide, for use in transducing cells, typically lymphocytes and illustrative embodiments T cells and/or NK cells. The replication incompetent recombinant retroviral particles can include an envelope protein. In some embodiments, the envelope protein can be a pseudotyping element. In some embodiments, the envelope protein can be an activation element. In some embodiments, the replication incompetent recombinant retroviral particles include both a pseudotyping element and an activation element. The replication incompetent recombinant retroviral particles can include any of the pseudotyping elements discussed elsewhere herein. In some embodiments, the replication incompetent recombinant retroviral particles can include any of the activation elements discussed elsewhere herein. In one aspect, provided herein is a replication incompetent recombinant retroviral particle (RIP) that includes a polynucleotide with nucleic acids that encode at least one of a CAR, a TCR, an LE, an anti-idiotype polypeptide, and a cytokine, and an inhibitory RNA provided in any of the aspects and embodiments herein. Such polypeptide typically includes nucleic acids that further encode at least one of a CAR, a TCR, an LE, a cytokine, and an inhibitory RNA. In some embodiments, the RIP includes a polynucleotide including: A) one or more transcriptional units operatively linked to a promoter active in T cells and/or NK cells, wherein the one or more transcriptional units encode at least one or anti-idiotype polypeptide, an engineered T cell receptor, or a chimeric antigen receptor (CAR); and B) a pseudotyping element and a T cell activation element on its surface, wherein the T cell activation element is not encoded by a polynucleotide in the replication incompetent recombinant retroviral particle. In some embodiments, the T cell activation element can be any of the activation elements discussed elsewhere herein. In illustrative embodiments, the T cell activation element can be anti-CD3 scFvFc. In any of the embodiments disclosed herein, the pseudotyping element may not be present. In another aspect, provided herein is a replication incompetent recombinant retroviral particle, including a polynucleotide including one or more transcriptional units operatively linked to a promoter active in T cells and/or NK cells, wherein the one or more transcriptional units encode a first polypeptide including an engineered T cell receptor or a chimeric antigen receptor (CAR) and a second polypeptide including a lymphoproliferative element. In some embodiments, the lymphoproliferative element can be a chimeric lymphoproliferative element. In illustrative embodiments, the lymphoproliferative element does not comprise IL-7 tethered to the IL-7 receptor alpha chain or a fragment thereof. In some embodiments the lymphoproliferative element does not comprise IL-15 tethered to the IL-2/IL-15 receptor beta chain. In some embodiments of any of the retroviral particle aspects or embodiments provided herein, or any other aspect that includes a retroviral particle, the engineered T cell receptor, CAR, or other transgene is expressed, displayed, and/or otherwise incorporated in the surface of the replication incompetent retroviral particle at a reduced level that is less than 70%, 60%, 50%, 40%, 30%, 20%, 10%, or 5% of the surface expression compared to when the transgene is expressed from an EF1-a or PGK promoter, and in illustrative embodiments, when the transgene is expressed from an EF1-a or PGK promoter in the absence of additional elements (such as degrons or inhibitory RNAs) to reduce such surface expression. In illustrative embodiments of any of the polynucleotide vector (e.g., RIP that include a membrane-bound hyaluronidase polypeptide) aspects provided herein, or any other aspect that includes a gene vector, the gene vector is substantially free of the protein transcript encoded by nucleic acid of the gene vector, and/or the RIPs do not express or comprise a detectable amount of the engineered T cell receptor or CAR on their surface, or express or comprise a reduced amount of the engineered T cell receptor or CAR on their surface.

In some aspects, provided herein is a replication incompetent recombinant retroviral particle that include a membrane-bound hyaluronidase polypeptide, comprising a polynucleotide comprising one or more transcriptional units operatively linked to a promoter active in T cells and/or NK cells, wherein the one or more transcriptional units encode a first polypeptide comprising a chimeric antigen receptor (CAR) and a second polypeptide comprising a lymphoproliferative element (LE), in illustrative embodiments a chimeric lymphoproliferative element (CLE), for example a constitutively active CLE. In illustrative embodiments, the chimeric lymphoproliferative element does not comprise a cytokine tethered to its cognate receptor or tethered to a fragment of its cognate receptor. As disclosed further herein, in some embodiments, the LE, such as the CLE, comprises a first lymphoproliferative element polypeptide (“LE polypeptide”) and a second LE polypeptide, wherein the first LE polypeptide has a different amino acid sequence from the second LE polypeptide and the first LE polypeptide and the second LE polypeptide are capable of, adapted to, and/or are configured to dimerize with each other. Such embodiments are called heterodimeric LEs herein. In illustrative embodiments, such an LE is a heterodimeric LE where the first LE polypeptide and the second LE polypeptide comprise a first extracellular dimerizing motif and a second extracellular dimerizing motif, respectively, that are capable of, adapted to, and/or configured to dimerize with each other. In illustrative embodiments, such an LE is a heterodimeric LE where the first LE polypeptide and the second LE polypeptide comprise a first intracellular dimerizing motif and a second intracellular dimerizing motif, respectively, that are capable of, adapted to, and/or configured to dimerize with each other.

Provided herein in some aspects, is a recombinant retroviral particle (RIP) that includes (i) a pseudotyping element capable of binding to a T cell and/or NK cell and facilitating membrane fusion of the recombinant retroviral particle thereto; (ii) a polynucleotide having one or more transcriptional units operatively linked to a promoter active in T cells and/or NK cells, wherein the one or more transcriptional units encode a first engineered signaling polypeptide having a chimeric antigen receptor that includes an antigen-specific targeting region, a transmembrane domain, and an intracellular activating domain, and a second engineered signaling polypeptide that includes at least one lymphoproliferative element; wherein expression of the first engineered signaling polypeptide and/or the second engineered signaling polypeptide are regulated by an in vivo control element; and (iii) an activation element on its surface, wherein the activation element is capable of binding to a T cell and/or NK cell and is not encoded by a polynucleotide in the recombinant retroviral particle, and wherein the RIP further includes a membrane-bound hyaluronidase polypeptide. In some embodiments, the promoter active in T cells and/or NK cells is not active in the packaging cell line or is only active in the packaging cell line in an inducible manner. In any of the embodiments disclosed herein, either of the first and second engineered signaling polypeptides can have a chimeric antigen receptor and the other engineered signaling polypeptide can have at least one lymphoproliferative element.

Various elements and combinations of elements that are included in replication incompetent, recombinant retroviral particles, and that can be included with RIPs that include a membrane-bound hyaluronidase polypeptide, are provided throughout this disclosure, such as, for example, pseudotyping elements, activation elements, and membrane bound cytokines, as well as nucleic acid sequences that are included in a genome of a replication incompetent, recombinant retroviral particle such as, but not limited to, nucleic acid sequences encoding an anti-idiotype polypeptide, nucleic acid sequences encoding a CAR; nucleic acid sequences encoding a lymphoproliferative element; nucleic acids encoding a cytokine; nucleic acid sequences encoding a control element, such as a riboswitch; a promoter, especially a promoter that is constitutively active or inducible in a T cell; and nucleic acid sequences encoding an inhibitory RNA molecule. Furthermore, various aspects provided herein, such as methods of making recombinant retroviral particles, methods for performing adoptive cell therapy, and methods for transducing T cells, produce and/or include replication incompetent, recombinant retroviral particles. Replication incompetent recombinant retroviruses that are produced and/or included in such methods themselves form separate aspects of the present disclosure as replication incompetent, recombinant retroviral particle compositions, which can be in an isolated form. Such compositions can be in dried down (e.g., lyophilized) form or can be in a suitable solution or medium known in the art for storage and use of retroviral particles.

Accordingly, as a non-limiting example, provided herein in another aspect, is a replication incompetent recombinant retroviral particle having in its genome a polynucleotide having one or more nucleic acid sequences operatively linked to a promoter active in T cells and/or NK cells that in some instances, includes a first nucleic acid sequence that encodes one or more (e.g., two or more) inhibitory RNA molecules directed against one or more RNA targets and a second nucleic acid sequence that encodes a chimeric antigen receptor, or CAR, as described herein, and wherein the RIP further includes a membrane-bound hyaluronidase polypeptide. In other embodiments, a third nucleic acid sequence is present that encodes at least one (e.g., 1, 2, 3, or 4) lymphoproliferative element described previously herein that is not an inhibitory RNA molecule. In certain embodiments, the polynucleotide incudes one or more riboswitches as presented herein, operably linked to the first nucleic acid sequence, the second nucleic acid sequence, and/or the third nucleic acid sequence, if present. In such a construct, expression of one or more inhibitory RNAs, the CAR, and/or one or more lymphoproliferative elements that are not inhibitory RNAs is controlled by the riboswitch. In some embodiments, two to 10 inhibitory RNA molecules are encoded by the first nucleic acid sequence. In further embodiments, two to six inhibitory RNA molecules are encoded by the first nucleic acid sequence. In illustrative embodiments, 4 inhibitory RNA molecules are encoded by the first nucleic acid sequence. In some embodiments, the first nucleic acid sequence encodes one or more inhibitory RNA molecules and is located within an intron. In certain embodiments, the intron includes all or a portion of a promoter. The promoter can be a Pol I, Pol II, or Pol III promoter. In some illustrative embodiments, the promoter is a Pol II promoter. In some embodiments, the intron is adjacent to and downstream of the promoter active in a T cell and/or NK cell. In some embodiments, the intron is EF1-α intron A.

Recombinant retroviral particle embodiments herein include those wherein the retroviral particle comprises a genome that includes one or more nucleic acids encoding one or more inhibitory RNA molecules and further includes a membrane-bound hyaluronidase polypeptide. Various alternative embodiments of such nucleic acids that encode inhibitory RNA molecules that can be included in a genome of a retroviral particle, including combinations of such nucleic acids with other nucleic acids that encode a CAR or a lymphoproliferative element other than an inhibitory RNA molecule, are included for example, in the inhibitory RNA section provided herein, as well as in various other paragraphs that combine these embodiments. Furthermore, various alternatives of such replication incompetent recombinant retroviruses can be identified by exemplary nucleic acids that are disclosed within packaging cell line aspects disclosed herein. A skilled artisan will recognize that disclosure in this section of a recombinant retroviral particle that includes a genome that encodes one or more (e.g., two or more) inhibitory RNA molecules, can be combined with various alternatives for such nucleic acids encoding inhibitory RNA molecules provided in other sections herein. Furthermore, a skilled artisan will recognize that such nucleic acids encoding one or more inhibitory RNA molecules can be combined with various other functional nucleic acid elements provided herein, as for example, disclosed in the section herein that focuses on inhibitory RNA molecules and nucleic acid encoding these molecules. In addition, the various embodiments of specific inhibitory RNA molecules provided herein in other sections can be used in recombinant retroviral particle aspects of the present disclosure.

Necessary elements of recombinant retroviral vectors, such as lentiviral vectors, and other vectors, are known in the art. These elements are included in the packaging cell line section and in details for making replication incompetent, recombinant retroviral particles provided in the Examples section and as illustrated in WO2019/055946. For example, lentiviral particles typically include a nucleic acid containing a packaging signal (e.g. psi) and a GAG polypeptide, surrounded by an envelope that includes an envelope protein that can include a targeting element, such as but not limited to a pseudotyping element. In some embodiments, lentiviral particles can include packaging elements, REV, GAG and POL, which can be delivered to packaging cell lines via one or more packaging plasmids, a pseudotyping element, various examples which are provided herein, which can be delivered to a packaging cell line via a pseudotyping plasmid, and a genome, which is produced by a polynucleotide that is delivered to a host cell via a transfer plasmid and which typically includes a packaging signal, and can further include one or more transcriptional units. In some embodiments, a lentiviral vector does not include a REV, POL, and/or INT polypeptide. This polynucleotide typically includes the viral LTRs and a psi packaging signal. The 5′ LTR can be a chimeric 5′ LTR fused to a heterologous promoter, which includes 5′ LTRs that are not dependent on Tat transactivation. The transfer plasmid can be self-inactivating, for example, by removing a U3 region of the 3′ LTR. In some non-limiting embodiments, Vpu, such as a polypeptide comprising Vpu (sometimes called a “Vpu polypeptide” herein) including but not limited to, Src-FLAG-Vpu, is packaged within the retroviral particle for any composition or method aspect and embodiment provided herein that includes a retroviral particle. In some non-limiting embodiments, Vpx, such as Src-FLAG-Vpx, is packaged within the retroviral particle. Not to be limited by theory, upon transduction of a T cells, Vpx enters the cytosol of the cells and promotes the degradation of SAMHD1, resulting in an increased pool of cytoplasmic dNTPs available for reverse transcription. In some non-limiting embodiments, Vpu and Vpx is packaged within the retroviral particle for any composition or method aspect and embodiment that includes a retroviral particle provided herein.

Retroviral particles (e.g., lentiviral particles) included in various aspects of the present disclosure are in illustrative embodiments, replication incompetent, especially for safety reasons for embodiments that include introducing cells transduced with such retroviral particles into a subject. A skilled artisan will understand that a minimal retroviral particle typically can include a viral envelope, Gag, and a nucleic acid containing a packaging motif (e.g., packaging element) for incorporation into retrovirus. The viral envelope is a lipid bilayer, which is derived from the host cell (e.g., packaging cell) membrane during viral budding. The packaging motif (which is also referred to herein as a packaging signal or a packaging element) can be natural or synthetic.

When replication incompetent retroviral particles are used to transduce a cell, retroviral particles are not produced from the transduced cell. Modifications to the retroviral genome are known in the art to assure that retroviral particles that include the genome are replication incompetent. However, it will be understood that in some embodiments for any of the aspects provided herein, replication competent recombinant retroviral particles can be used. In some non-limiting embodiments, the retroviral particles lack a functional integrase. In these and other embodiments, the retroviral RNA is reverse transcribed, but does not integrate into the genome of the host cell (e.g., target cell). In some non-limiting embodiments, retroviral particles lack a functional reverse polymerase. In these and other embodiments, the retroviral RNA is neither reverse transcribed nor integrated into the genome of the target cell. In such embodiments, the retroviral RNA is present in the target cell, and polypeptides can be transcribed from its sequence, or the retroviral RNA can be functional as an RNA, for example, as an inhibitory RNA.

A skilled artisan will recognize that the functional elements discussed herein can be delivered to packaging cells and/or to target cells, for example, T and/or NK cells, using different types of vectors, such as expression vectors. Illustrative aspects of the present disclosure utilize retroviral vectors, and in some particularly illustrative embodiments lentiviral vectors. Other suitable expression vectors can be used to achieve certain embodiments herein, and a skilled artisan will understand how to use other vectors instead of retroviral particles with the aspects and embodiments disclosed herein. Such expression vectors include, but are not limited to, viral vectors (e.g., viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, all of which are incorporated by reference in their entireties); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90: 10613-10617, all of which are incorporated by reference in their entireties); SV40; herpes simplex virus; rhabdovirus (e.g., Vesicular stomatitis virus); reovirus; senecavirus; enterovirus; Semliki Forest virus; maraba virus; Coxsackievirus A21; Influenza A; or a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus), for example a gamma retrovirus; or human immunodeficiency virus (see, e.g., Miyoshi et al., PNAS 94:10319 23, 1997; Takahashi et al., J Virol 73:7812 7816, 1999, all of which are incorporated by reference in their entireties); and the like.

As disclosed herein, replication incompetent recombinant retroviral particles are a common tool for gene delivery (Miller, Nature (1992) 357:455-460). The ability of replication incompetent recombinant retroviral particles to deliver an unrearranged nucleic acid sequence into a broad range of rodent, primate and human somatic cells makes replication incompetent recombinant retroviral particles well suited for transferring genes to a cell. In some embodiments, the replication incompetent recombinant retroviral particles that include a membrane-bound hyaluronidase polypeptide, can be derived from the Alpharetrovirus genus, the Betaretrovirus genus, the Gammaretrovirus genus, the Deltaretrovirus genus, the Epsilonretrovirus genus, the Lentivirus genus, or the Spumavirus genus. There are many retroviruses suitable for use in the methods disclosed herein. For example, murine leukemia virus (MLV), human immunodeficiency virus (HIV), equine infectious anaemia virus (EIAV), mouse mammary tumor virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) can be used. A detailed list of retroviruses may be found in Coffin et al (“Retroviruses” 1997 Cold Spring Harbor Laboratory Press Eds: J M Coffin, S M Hughes, H E Varmus pp 758-763). Details on the genomic structure of some retroviruses may be found in the art. By way of example, details on HIV may be found from the NCBI Genbank (i.e., Genome Accession No. AF033819).

In illustrative embodiments, the replication incompetent recombinant retroviral particles that include a membrane-bound hyaluronidase polypeptide, can be derived from the Lentivirus genus. In some embodiments, the replication incompetent recombinant retroviral particles can be derived from HIV, SIV, or FIV. In further illustrative embodiments, the replication incompetent recombinant retroviral particles can be derived from the human immunodeficiency virus (HIV) in the Lentivirus genus. Lentiviruses are complex retroviruses which, in addition to the common retroviral genes gag, pol and env, contain other genes with regulatory or structural function. The higher complexity enables the lentivirus to modulate the life cycle thereof, as in the course of latent infection. A typical lentivirus is the human immunodeficiency virus (HIV), the etiologic agent of AIDS. in vivo, HIV can infect terminally differentiated cells that rarely divide, such as lymphocytes and macrophages.

In illustrative embodiments, replication incompetent recombinant retroviral particles provided herein contain Vpx polypeptide.

In some embodiments, replication incompetent recombinant retroviral particles provided herein comprise and/or contain Vpu polypeptide.

In illustrative embodiments, a retroviral particle is a lentiviral particle. Such retroviral particle typically includes a retroviral genome within a capsid which is located within a viral envelope.

In some embodiments, the HIV RREs and the polynucleotide region encoding HIV Rev can be replaced with N-terminal RGG box RNA binding motifs and a polynucleotide region encoding ICP27. In some embodiments, the polynucleotide region encoding HIV Rev can be replaced with one or more polynucleotide regions encoding adenovirus E1B 55-kDa and E4 Orf6.

In some embodiments, the isolated replication incompetent retroviral particles are a large-scale batch contained in a large-scale container. Such large-scale batch can have titers, for example of 10⁶-10⁸ TU/ml and a total batch size of between 1×10¹⁰ TU and 1×10¹³ TU, 1×10¹¹ TU and 1×10¹³ TU, 1×10¹² TU and 1×10¹³ TU, 1×10¹⁰ TU and 5×10¹² TU, or 1×10¹¹ TU and 5×10¹² TU. In illustrative embodiments, retroviral particles for any aspect or embodiment provided herein are substantially pure, as discussed in more detail herein.

In some embodiments, a recombinant retroviral particle (RIP) formulation is provided, that include a recombinant retroviral particle, or a plurality thereof, that includes a membrane-bound hyaluronidase, in a solution, for example in a buffer, or in an excipient that is suitable for vial delivery for example to a subject. The RIP or RIPs in such formulations in illustrative embodiments include a polynucleotide comprising one or more transcriptional units, wherein each of the one or more transcriptional units is operatively linked to a promoter active in a target cell,

### Recombinant Viral Particles

In some embodiments, DNA-containing viral particles, in illustrative embodiments recombinant viral particles, are provided and/or utilized instead of, or in combination with recombinant retroviral particles. Such DNA-containing viral particles typically include a membrane-bound hyaluronidase polypeptide. In some embodiments, the membrane-bound hyaluronidase polypeptide is a chimeric hyaluronidase polypeptide disclosed herein. The membrane-bound hyaluronidase polypeptide typically has hyaluronidase activity and in some embodiments is a hyaluronidase polypeptide comprising an amino acid sequence with at least 75%, 80%, 90%, 95%, 96%, 97%, 98%, or 99% sequence identity to any one of SEQ ID Nos:544-1003. Such recombinant viral particles can be adenoviruses, adeno-associated viruses, herpesviruses, cytomegaloviruses, poxviruses, avipox viruses, influenza viruses, vesicular stomatitis virus (VSV), or Sindbis virus. In illustrative embodiments, such recombinant viral particles are replication incompetent. A skilled artisan will appreciate how to modify the methods disclosed herein for use with different viruses, viral particles, retroviruses, and retroviral particles. Where viral particles are used that include a DNA genome, a skilled artisan will appreciate that functional units can be included in such genomes to induce integration of all or a portion of the DNA genome of the viral particle into the genome of a cell, such as for example a T cell, transduced with such virus. In some embodiments, a recombinant viral particle formulation is provided, that include a viral particle, recombinant viral particle, or a plurality thereof, that includes a membrane-bound hyaluronidase, in a solution, for example in a buffer, or in an excipient that is suitable for vial delivery for example to a subject. The viral particles or recombinant viral particles in such formulations in illustrative embodiments include a polynucleotide comprising one or more transcriptional units, wherein each of the one or more transcriptional units is operatively linked to a promoter active in a target cell.

### Membrane-Bound and Soluble Hyaluronidase

Some embodiments of the method and composition aspects provided herein, include membrane-bound hyaluronidase (i.e., a membrane-bound hyaluronidase polypeptide), or polynucleotides encoding membrane-bound hyaluronidase. Such membrane bound hyaluronidase in illustrative embodiments is associated, attached, or on one or more membranes of any of the packaging cells and/or RIPs provided herein. Such membrane bound hyaluronidase in illustrative embodiments, is a chimeric hyaluronidase polypeptide disclosed herein, for example those disclosed in SEQ ID NOs:544-1003. Not to be limited by theory, hyaluronidases facilitate the dispersion and absorption of injected drugs by enabling large volume subcutaneous delivery, particularly beyond the typically administered 2 ml or less volume, and may increase the lymphatic absorption profile of RIPs injected subcutaneously. In certain embodiments, the hyaluronidase is a hyaluronidase glycoprotein. The hyaluronidase may also be recombinant, and furthermore certain hyaluronidases may be membrane-bound via their endogenous GP1 anchors to incorporate in the RIP. Alternatively, the hyaluronidase catalytic domain may be a fusion protein at the carboxy terminus with a heterologous membrane attachment sequence. In some embodiments, the heterologous membrane attachment sequence is a heterologous GPI anchor attachment sequence. The hyaluronidase may be human or non-human derived. Certain hyaluronidases, such as HYAL5 are active in some species such as bovine but are pseudogenes in humans. The heterologous GPI anchor attachment sequence can also be derived from any known GPI-anchored protein. In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF. The hyaluronidase can be any hyaluronidase enzyme found in U.S. Pat. No. 7,767,429, incorporated by reference herein, in its entirety as well as those that are membrane attached. In illustrative examples, the hyaluronidase is bovine HYAL5 or a catalytically active portion thereof, but that does not include the entire sequence of amino acids set forth in SEQ ID NOs:422, 470, or 471, or in some embodiments, SEQ ID NO:422. In some embodiments, the hyaluronidase includes a sequence of amino acids that has a least 70%, 80%, 85%, 90%, 95% or 100% sequence identity to SEQ ID NO:422 or 423. In some embodiments, the hyaluronidase glycoprotein contains amino acids 1-477, 36-477, 1-478, 36-478, 1-479, 36-479, 1-480, 36-480, 1-481, 36-481, 1-482, 36-482, 1-483 or 36-483 of SEQ ID NO:422, or contains amino acid substitutions in the sequence of amino acids set forth as amino acids 1-477, 36-477, 1-478, 36-478, 1-479, 36-479, 1-480, 36-480, 1-481, 36-481, 1-482, 36-482, 1-483 or 36-483 of SEQ ID NO:422, whereby the amino-acid substituted hyaluronidase glycoprotein consists of a sequence of amino acids that has at least 95% amino acid sequence identity with the sequence of amino acids set forth as amino acids 1-477, 36-477, 1-478, 36-478, 1-479, 36-479, 1-480, 36-480, 1-481, 36-481, 1-482, 36-482, 1-483 or 36-483 of SEQ ID NO:422, wherein the glycoprotein contains at least one glycosylation moiety that is covalently attached to an asparagine (N) residue of the polypeptide. In some embodiments, the hyaluronidase is a combination of chimeric proteins (i.e., a combination of consecutive stretches of amino acids wherein the consecutive stretches are from different hyaluronidases from the same or different species) from any of the hyaluronidases HYAL1, PH20, HYAL2, HYAL3, or HYAL5 with catalytic domains and carboxy terminal EGF repeat domains. Some embodiments include a nucleic acid vector that encodes for hyaluronidase that encodes a membrane bound hyaluronidase enzyme. Such a vector may be used in methods to transfect viral packaging host cells. The viral packaging host cells may express the hyaluronidase fusion protein on its cell membrane. During viral packaging, portions of the cell membrane that comprise the hyaluronidase fusion protein will be incorporated into the virus membrane. In further embodiments, hyaluronidase enzymatic domains and carboxy terminal domains may also be produced as a chimera with a transmembrane domain to facilitate incorporation into the viral plasma membrane. Transmembrane domains with short carboxy termini are preferred to minimize interference with the intracellular viral matrix proteins. In some embodiments, the transmembrane domain can be GP120, or any other transmembrane protein that interacts favorably with the matrix proteins. In other cases transmembrane domain can be PDGFR or other membrane domain that expresses well without a long cytoplasmic domain. In other cases the transmembrane domain naturally accumulates in lipid rafts.

Some embodiments of the method and composition aspects provided herein include soluble hyaluronidase (i.e., a soluble hyaluronidase polypeptide), or polynucleotides encoding soluble hyaluronidase. Some embodiments of the method and composition aspects provided herein include membrane-bound hyaluronidase (i.e., a membrane-bound hyaluronidase polypeptide), or polynucleotides encoding soluble or membrane-bound hyaluronidase. Such membrane bound hyaluronidase in illustrative embodiments is associated, attached, or on one or more membranes of any of the packaging cells, viruses such as RIPs, or modified cells provided herein. Not to be limited by theory, hyaluronidases facilitate the dispersion and absorption of small molecule therapeutics and biologics such as viruses and cells through tissue, and can increase their lymphatic absorption profile when injected subcutaneously. Accordingly, some formulations, delivery solutions, delivery reagents, and pharmaceutical compositions herein include a hyaluronidase polypeptide provided herein, and one or more additional components. In some embodiments, the additional component is a biologic and/or a small molecule therapeutic. For example, the biologic can be a virus, a retrovirus, or a cell, any of which in illustrative embodiments, can be recombinant. Not to be limited by theory, in some embodiments herein, by attaching hyaluronidase to the biologic, such as to the surface of a retrovirus, the hyaluronidase and the retorivurs would remain co-located and thereby enhance spreading and dispersion.

In some embodiments, the one or more additional components can be include adalimumab, agalsidase beta, aldesleukin, alefacept, ampicillin, anakinra, antipoliomyelitic vaccine, anti-thymocyte, azithromycin, becaplermin, caspofungin, cefazolin, cefepime, cefotetan, ceftazidime, ceftriaxone, cetuximab, cilastatin, clavulanic acid, clindamycin, darbepoetin alfa, deaclizumab, diphtheria toxoid, efalizumab, epinephrine, erythropoietin alpha, etanercept, filgrastim, fluconazole, follicle-stimulating hormones such as follitropin alphas and follitropin beta, fosphenyloin, fluconazole, gadodiamide, gadopentetate, gatifloxacin, glatiramer, Gm-csfr™, goserelin, granisetron, haemophilus influenza b, haloperidol, hepatitis a vaccine, hepatitis b vaccine, ibritumomab tiuxetan, immunoglobulin, influenza virus vaccine, infliximab, insulin, insulin glargine, interferon alfa-2a, interferon alfa-2b, interferon alfacon-1, interferon alfa-n3, interferon beta, interferon beta-1a, interferon gamma, iodixanol, iohexyl, iopamidol, ioversol, ketorolac, laronidase, levofloxacin, lidocaine, linezolid, lorazepam, measles vaccine, measles-mumps-rubella virus vaccine, medroxyprogesterone, meropenem, methylprednisolone, midazolam, morphine, octreotide, omalizumab, ondansetron, palivizumab, pantoprazole, pegaspargase, pegfilgrastim, peg-interferon alfa-2a, peg-interferon alfa-2b, pegvisomant, pertussis vaccine, piperacillin, pneumococcal vaccines and pneumococcal conjugate vaccine, promethazine, reteplase, somatropin, sulbactam, sumatriptan, tazobactam, tenecteplase, tetanus purified toxoid, ticarcillin, tositumomab, triamcinolone acetonide, vancomycin, or varicella vaccines.

In some embodiments, the one or more additional components can include a blood modifier. In some embodiments, the blood modifier can include antihemophilic factors, anti-inhibitor coagulant complexes, antithrombin Ills, coagulations Factor VIIs, coagulation Factor VIIs, coagulation Factor IXs, plasma protein fractions, antiplatelet agents, anagrelides, cilostazols, clopidogrel bisulfates, dipyridamoles, epoprostenols, eptifibatides, tirofibans, colony stimulating factors (CSFs), erythropoiesis stimulators, hemostatics, albumins, combinations of antihemophilic factors and plasma, desmopressin acetates, immune globulins, hepatitis B immune globulins, thrombin inhibitors, lepirudin, drotecogin alfas, anticoagulants, and warfarins.

In some embodiments, the one or more additional components can include acetazolamides, acyclovirs, adipiodones, alatrofloxacins, aldesleukins, alemtuzumabs, alfentanils, allergenic extracts, allopurinols, alpha 1-proteinase inhibitors, alprostadils, amifostines, amikacins, aminoacids, aminocaproic acids, aminophyllines, amitriptylines, amobarbitals, amrinones, anakinras, analgesics, anti-poliomyelitic vaccines, anti-rabic serums, anti-tetanus immunoglobulins, antithrombin iiis, antivenom serums, argatrobans, arginines, arsenics, ascorbic acids, asparaginases, atenolols, atracuriums, atropines, aurothioglucoses, azathioprines, azithromycins, aztreonams, bacillus calmette-guerin (bcg) vaccines, bacitracins, baclofens, basiliximabs, benzoic acids, benztropines, betamethasones, biotins, bivalirudins, bleomycins, botulism antitoxins, bretyliums, bumetanides, bupivacaines, buprenorphines, busulfans, butorphanols, calcitonins, calcitriols, calciums, capreomycins, carboprosts, carmustines, carnitines, caspofungins, cefamandoles, cefazolins, cefoperazones, cefotaximes, cefotetans, cefoxitins, ceftazidimes, ceftizoximes, cefuroximes, chloramphenicols, chloroprocaines, chloroquines, chlorothiazides, chlorpromazines, chondroitinsulfuric acids, choriogonadotropin alfas, chromiums, cidofovirs, cimetidines, ciprofloxacins, cisatracuriums, cisplatins, cladribines, clavulanic acids, clindamycins, clonidines, codeines, colchicines, colistins, collagens, corticorelin ovine triflutates, corticotrophins, cosyntropins, cyanocobalamins, cyclophosphamides, cyclosporines, cysteines, cytarabines, dacarbazines, dacliximabs, dactinomycins, dalfopristins, dalteparins, danaparoids, dantrolenes, daunorubicins, deferoxamines, denileukin diftitoxs, desmopressins, dexamethasones, dexmedetomidines, dexpanthenols, dexrazoxanes, dextrans, diatrizoic acids, diazepams, diazoxides, dicyclomines, digibinds, digoxins, dihydroergotamines, diltiazems, diphenhydramines, diphteria, tetanus and pertussis vaccines, diptheria antitoxins, dipyridamoles, dobutamines, dopamines, doxacuriums, doxaprams, doxercalciferols, doxycyclines, properidols, drotrecogin alfas, dyphyllines, edetic acids, edrophoniums, enalaprilats, ephedrines, epoprostenols, ergocalciferols, ergonovines, ertapenems, erythromycins, esmolols, estradiols, estrogenics, ethacrynic acids, ethanolamines, ethanols, ethiodized oils, etidronic acids, etomidates, etoposides, factor viis, famotidines, fenoldopams, fentanyls, floxuridines, fludarabines, flumazenils, fluoresceins, fluorouracils, fluphenazines, folic acids, follicle-stimulating hormones, fomepizoles, fomivirsens, fondaparinuxs, foscamets, fosphenyloins, fulvestrants, furosemides, gadoteridols, gadoversetamides, ganciclovirs, gatifloxacins, gemtuzumab ozogamicins, gentamicins, glucagons, glucoses, glycines, glycopyrrolates, gonadorelins, gonadotropin chorionics, haemophilus b polysaccarides, haloperidols, hemins, hemophilus influenza vaccines, hepatitis vaccines, herbals, histamines, hydralazines, hydrocortisones, hydromorphones, hydroxocobalamins, hydroxyzines, hyoscyamines, ibutilides, idarubicins, ifosfamides, imiglucerases, immunoglobulins, indigo carmines, indomethacins, interferon alfa-conls, interferon alfa-2as, interferon alfa-n3s, interferon beta-1 as, iodides, iopromides, iothalamic acids, ioxaglic acids, ioxilans, iron dextran injections, isoniazids, isoproterenols, japanese encephalitis vaccines, kanamycins, ketamines, ketorolacs, labetalols, lepirudins, leucovorins, leuprolides, levobupivacaines, levothyroxines, lidocaines, lincomycins, linezolids, liothyronines, lorazepams, luteinising hormones, lyme disease vaccines, mangafodipirs, mannitols, measles virus, mechlorethamines, melphalans, meningococcal polysaccharide vaccines, meperidines, mepivacaines, meropenems, mesnas, mesoridazines, metaraminols, methadones, methocarbamols, methohexitals, methotrexates, methyldopates, methylergonovines, metoclopramides, metoprolols, metronidazoles, midazolams, minocyclines, mithramycins, mitomycins, mitoxantrones, mivacuriums, morrhuic acids, moxifloxacins, mumps viruses, muromonab-cd3 s, mycophenolate mofetils, nafcillins, nalbuphines, nalmefenes, naloxones, nandrolones, neostigmines, niacinamides, nicardipines, nitroglycerins, nitroprussides, norepinephrines, oprelvekins, orphenadrines, oxacillins, oxaliplatins, oxymorphones, oxytetracyclines, oxytocins, pancuroniums, panthenols, pantothenic acids, papaverines, pegaspargases, peginterferon alfa 2as, penicillin gs, pentamidines, pentazocines, pentobarbitals, pentostatins, perflutrens, perphenazines, pertussis vaccines, phenobarbitals, phentolamines, phenylephrines, phenyloins, physostigmines, phytonadiones, pneumococcal vaccines, polymyxin bs, porfimers, pralidoximes, prilocalnes, procainamides, procaines, prochlorperazines, progesterones, promethazines, propranolols, pyridostigmine hydroxides, pyridoxines, quinidines, quinupristins, rabies immunoglobulins, rabies vaccines, ranitidines, rasburicases, remifentanils, riboflavins, rifampins, ropivacaines, rubella vaccines, samariums, scopolamines, seleniums, sermorelins, sincalides, somatrems, spectinomycins, streptokinases, streptomycins, streptozocins, succinylcholines, sufentanils, sulbactams, sulfamethoxazoles, sumatriptans, tacrolimuss, teniposides, terbutalines, teriparatides, testosterones, tetanus antitoxins, tetracaines, tetradecyl sulfates, theophyllines, thiamines, thiethylperazines, thiopentals, thyroid stimulating hormones, ticarcillins, tinzaparins, tirofibans, tobramycins, tolazolines, tolbutamides, topotecans, torsemides, tranexamic acids, treprostinils, triamcinolone acetonides, triamcinolone hexacetonides, triamcinolones, triethylenethiophosphoramides (thiotepa), trifluoperazines, trimethobenzamides, trimethoprims, trimetrexates, triptorelins, tromethamines, tuberculins, typhoid vaccines, urofollitropins, urokinases, valproic acids, valrubicins, varicella zoster immunoglobulins, vasopressins, vecuroniums, verapamils, vinblastines, vincristines, voriconazoles, warfarins, yellow fever vaccines, zidovudines, zincs, or ziprasidone hydrochlorides.

The hyaluronidase in any aspect or embodiments herein may be a bacterial hyaluronidase (EC 4.2.99.1), a hyaluronidase from leeches, other parasites and crustaceans (EC 3.2.1.36), or it may be a mammalian-type hyaluronidase (EC 3.2.1.35). The hyaluronidase may be human or non-human derived. The hyaluronidase may be non-human primate derived. In some embodiments, the hyaluronidase is derived from a Pan troglodytes, Pan paniscus, Gorilla gorilla, Pongo abeli, Hylobates lar, Symphalangus syndactylus, Nomascus leucogenys, Nasalis larvatur, Colobus angolensis, Colobus guerza, Semnopithecus entellus, Colobus angolensis, Pygathrix nemaeus, Rhinopithecus roxellana, Sanguinus oedipus, Cercopithecus mitis, Macaca nemestrina, Aotus nancymaae, Sanguinus fuscicollis, Callimico goeldii, Erythrocebus patas, Chlorocebus aethiops, Papio hamadryas, or a Cholorocebus sabaeus hyaluronidase.

In some embodiments, the hyaluronidase is derived from a bovine, murine, rat, rabbit, or an ovine hyaluronidase. In some embodiments, the hyaluronidase is human derived. Exemplary human hyaluronidases include Hyal-1, Hyal-2, Hyal-3, Hyal-4, and PH20. HYALP1 is apseudogene in humans, however some orthologs of HYALP1 encode active enzymes, such as the mouse ortholog. Accordingly, hyaluronidase polypeptides herein can have a sequence that is at least 80, 85, 90, 95, 96, 97, 98, 99, or 100% identical to a pseudogion or to one or more of regions 1 to 8 (as provided herein) thereof, typically that together result in a hyaluronidase polypeptide having hyaluronidase activity. In some embodiments, the hyaluronidase is bovine derived. Exemplary bovine hyaluronidases include Hyal-1 and PH20. In some embodiments, the hyaluronidase is a chimera comprising hyaluronidase sequences from more than one species. In illustrative embodiments the hyaluronidase has the ability to enzymatically catalyze the cleavage of hyaluronic acid at acidic pH and is thus acid-active. In some embodiments the hyaluronidase has the ability to enzymatically catalyze the cleavage of hyaluronic acid at neutral pH and is thus neutral-active.

Hyaluronidases contemplated in aspects and embodiments herein (including, but not limited to Hyal-1, Hyal-2, Hyal-3, Hyal-4, Hyal-5, and PH20) include precursor hyaluronidase polypeptides and mature hyaluronidase polypeptides (such as those in which a signal sequence has been removed), truncated forms thereof that have enzymatic activity, and include allelic variants and species variants, variants encoded by splice variants, chimeras, and other variants, including polypeptides that have at least 80%, 85%, 90%, 91%, 92%, 93%, 94%, 95%, 96%, 97%, 98%, 99% or 100% sequence identity to the polypeptides set forth in SEQ ID NOs:422-481 or SEQ ID NOs:1040-1077, or to any of the engineered hyaluronidase variants disclosed herein including those set forth in SEQ ID NOs:544-1003. In some embodiments, the hyaluronidase includes a polypeptide with at least 90% sequence identity to any one of the amino acid sequences in SEQ ID NOs:422-481, or SEQ ID NOs:1040-1077, or to SEQ ID NOs:544-1003. In some embodiments, the hyaluronidase includes a polypeptide with at least 95% sequence identity to any one of the amino acid sequences in SEQ ID NOs:422-481, or SEQ ID NOs:1040-1077, or to SEQ ID NOs:544-1003. A hyaluronidase can be any hyaluronidase enzyme found in any of U.S. Pat. Nos. 7,767,429, 8,927,249, US 2021/0155913, or US 2022/0289864, each incorporated by reference herein, in their entirety, as well as those that are membrane soluble. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

In some embodiments, the hyaluronidase is Hyal-1 derived. Examples of such embodiments include polypeptides that have at least 80, 85, 90, 95, 96, 97, 98, 99, or 100% sequence identity to any of the Hyal-1 polypeptides provided herein, or to one or more of regions 1 to 8 (as provided herein) of a Hyal-1 polypeptide provided herein. Typically, such Hya-1 derived polypeptide has hyaluronidase activity. Hyal-1 includes those of any origin including, human, non-human primates, bovine, murine, rat, rabbit, or an ovine origin. Exemplary precursor Hyal-1 polypeptides include those from human (SEQ ID NO:434), chimpanzee (SEQ ID NO:435), Rhesus monkey (SEQ ID NO:436), Cynomolgus monkey (SEQ ID NO:437), cow (SEQ ID NO:438), mouse (SEQ ID NO:439), rat (SEQ ID NO:440), rabbit (SEQ ID NO:441), and sheep (SEQ ID NO:442). In some embodiments, the Hyal-1 has either an N-terminus deletion of amino acid residues 1-21, 1-22, 1-23, 1-24, 1-25, 1-26, 1-27, 1-28, or 1-29 of SEQ ID NO:434; and/or a C-terminus deletion of amino acid residues 431-435, 432-435, 433-435, 434-435, or 435 wherein the numbering is by reference to human Hyal-1 SEQ ID NO:434, and includes the corresponding Hyal-1 fragments of orthologs to these human Hyal-1 fragments. In some embodiments, the hyaluronidase fragment has at least 85%, 90%, 91%, 92%, 93%, 94%, 95%, 96%, 97%, 98%, 99% sequence identity to amino acid residues 22-431, 22-432, 22-433, 22-434, or 22-435 of SEQ ID NO:434, or to the corresponding Hyal-1 fragments of orthologs to these human Hyal-1 fragments. In some embodiments, the hyaluronidase fragment has at least 95% sequence identity to amino acid residues 22-431, 22-432, 22-433, 22-434, or 22-435 of SEQ ID NO:434, or to the corresponding Hyal-1 fragments of orthologs to these human Hyal-1 fragments.

In some embodiments, the hyaluronidase is Hyal-2 derived. Examples of such embodiments include polypeptides that have at least 80, 85, 90, 95, 96, 97, 98, 99, or 100% sequence identity to any of the Hyal-2 polypeptides provided herein, or to one or more of regions 1 to 8 (as provided herein) of a Hyal-2 polypeptide provided herein. Typically, such Hya-2 derived polypeptide has hyaluronidase activity. Hyal-2 includes those of any origin including, human, non-human primates, bovine, murine, rat, rabbit, or an ovine origin. Exemplary precursor Hyal-2 polypeptides include those from human (SEQ ID NO:443), chimpanzee (SEQ ID NO:444), Rhesus monkey (SEQ ID NO:445), Cynomolgus monkey (SEQ ID NO:446), cow (SEQ ID NO:447), mouse (SEQ ID NO:448), rat (SEQ ID NO:449), rabbit (SEQ ID NO:450), and sheep (SEQ ID NO:451). In some embodiments, the Hyal-2 has either an N-terminus deletion of amino acid residues 1-20, 1-21, 1-22, 1-23, 1-24, 1-25, 1-26, 1-27, or 1-28 of SEQ ID NO:443 and/or a C-terminus deletion of amino acid residues 440-473, 441-473, 442-473, 443-473, 444-473, 445-473, 446-473, 447-473, 448-473, 449-473, 450-473, 451-473, 452-473, 453-473, 454-473, 455-473, 456-473, 457-473, 458-473, 459-473, 460-473, 461-473, 462-473, 463-473, 464-473, 465-473, 466-473, 467-473, 468-473, 469-473, 470-473, 471-473, 472-473, or 473 wherein the numbering is by reference to human Hyal-2 (SEQ ID NO:443), and includes the corresponding Hyal-2 fragments of orthologs to these human Hyal-2 fragments. In some embodiments, the hyaluronidase fragment has at least 85%, 90%, 91%, 92%, 93%, 94%, 95%, 96%, 97%, 98%, 99% sequence identity to amino acid residues 21-440, 21-441, 21-442, 21-443, 21-444, 21-445, 21-446, 21-447, 21-448, 21-449, 21-450, 21-451, 21-452, 21-453, 21-454, 21-455, 21-456, 21-457, 21-458, 21-459, 21-460, 21-461, 21-462, 21-463, 21-464, 21-465, 21-466, 21-467, 21-468, 21-469, 21-470, 21-471, 21-472, or 21-473 of (SEQ ID NO:443), or to the corresponding Hyal-2 fragments of orthologs to these human Hyal-2 fragments. In some embodiments, the hyaluronidase fragment has at least 95% sequence identity to amino acid residues 21-440, 21-441, 21-442, 21-443, 21-444, 21-445, 21-446, 21-447, 21-448, 21-449, 21-450, 21-451, 21-452, 21-453, 21-454, 21-455, 21-456, 21-457, 21-458, 21-459, 21-460, 21-461, 21-462, 21-463, 21-464, 21-465, 21-466, 21-467, 21-468, 21-469, 21-470, 21-471, 21-472, or 21-473 of (SEQ ID NO:443), or to the corresponding Hyal-2 fragments of orthologs to these human Hyal-2 fragments.

In some embodiments, the hyaluronidase is Hyal-3 derived. Examples of such embodiments include polypeptides that have at least 80, 85, 90, 95, 96, 97, 98, 99, or 100% sequence identity to any of the Hyal-3 polypeptides provided herein, or to one or more of regions 1 to 8 (as provided herein) of a Hyal-3 polypeptide provided herein. Typically, such Hya-3 derived polypeptide has hyaluronidase activity. Hyal-3 includes those of any origin including, human, non-human primates, bovine, murine, rat, rabbit, or an ovine origin. Exemplary precursor Hyal-3 polypeptides include those from human (SEQ ID NO:452), chimpanzee (SEQ ID NO:453), Rhesus monkey (SEQ ID NO:454), Cynomolgus monkey (SEQ ID NO:455), cow (SEQ ID NO:456), mouse (SEQ ID NO:457), rat (SEQ ID NO:458), rabbit (SEQ ID NO:459), and sheep (SEQ ID NO:460). In some embodiments, the Hyal-3 has either an N-terminus deletion of amino acid residues residues 1-20, 1-21, 1-22, 1-23, 1-24, 1-25, 1-26, 1-27, or 1-28 of SEQ ID NO:452; and/or a C-terminus deletion of amino acid residues 407-417, 408-417, 409-417, 410-417, 411-417, 412-417, 413-417, 414-417, 415-417, 416-417, or 417 wherein the numbering is by reference to human Hyal-3 SEQ ID NO:452, and includes the corresponding Hyal-3 fragments of orthologs to these human Hyal-3 fragments. In some embodiments, the hyaluronidase fragment has at least 85%, 90%, 91%, 92%, 93%, 94%, 95%, 96%, 97%, 98%, 99% sequence identity to amino acid residues 21-407, 21-408, 21-409, 21-410, 21-411, 21-412, 21-413, 21-414, 21-415, 21-416, or 21-417 of SEQ ID NO:452, or to the corresponding Hyal-3 fragments of orthologs to these human Hyal-3 fragments. In some embodiments, the hyaluronidase fragment has at least 95% sequence identity to amino acid residues 21-407, 21-408, 21-409, 21-410, 21-411, 21-412, 21-413, 21-414, 21-415, 21-416, or 21-417 of SEQ ID NO:452, or to the corresponding Hyal-3 fragments of orthologs to these human Hyal-3 fragments.

In some embodiments, the hyaluronidase is Hyal-4 derived. Examples of such embodiments include polypeptides that have at least 80, 85, 90, 95, 96, 97, 98, 99, or 100% sequence identity to any of the Hyal-4 polypeptides provided herein, or to one or more of regions 1 to 8 (as provided herein) of a Hyal-4 polypeptide provided herein. Typically, such Hya-4 derived polypeptide has hyaluronidase activity. Hyal-4 includes those of any origin including, human, non-human primates, bovine, murine, rat, rabbit, or an ovine origin. Exemplary precursor Hyal-4 polypeptides include those from human (SEQ ID NO:461), chimpanzee (SEQ ID NO:462), Rhesus monkey (SEQ ID NO:463), Cynomolgus monkey (SEQ ID NO:464), cow (SEQ ID NO:465), mouse (SEQ ID NO:466), rat (SEQ ID NO:467), rabbit (SEQ ID NO:468), and sheep (SEQ ID NO:469). In some embodiments, the Hyal-4 has either an N-terminus deletion of amino acid residues 1-34, 1-35, 1-36, 1-37, 1-38, 1-39, 1-40, 1-41, or 1-42 of SEQ ID NO:461; and/or a C-terminus deletion of amino acid residues 448-481, 449-481, 450-481, 451-481, 452-481, 453-481, 454-481, 455-481, 456-481, 457-481, 458-481, 459-481, 460-481, 461-481, 462-481, 463-481, 464-481, 465-481, 466-481, 467-481, 468-481, 469-481, 470-481, 471-481, 472-481, 473-481, 474-481, 475-481, 476-481, 477-481, 478-481, 479-481, 480-481, or 481 wherein the numbering is by reference to human Hyal-4 SEQ ID NO:461, and includes the corresponding Hyal-4 fragments of orthologs to these human Hyal-4 fragments. In some embodiments, the hyaluronidase fragment has at least 85%, 90%, 91%, 92%, 93%, 94%, 95%, 96%, 97%, 98%, 99% sequence identity to amino acid residues 35-448, 35-449, 35-450, 35-451, 35-452, 35-453, 35-454, 35-455, 35-456, 35-457, 35-458, 35-459, 35-460, 35-461, 35-462, 35-463, 35-464, 35-465, 35-466, 35-467, 35-468, 35-469, 35-470, 35-471, 35-472, 35-473, 35-474, 35-475, 35-476, 35-477, 35-478, 35-479, 35-480, or 35-481 of SEQ ID NO:461, or to the corresponding Hyal-4 fragments of orthologs to these human Hyal-4 fragments. In some embodiments, the hyaluronidase fragment has at least 95% sequence identity to amino acid residues 35-448, 35-449, 35-450, 35-451, 35-452, 35-453, 35-454, 35-455, 35-456, 35-457, 35-458, 35-459, 35-460, 35-461, 35-462, 35-463, 35-464, 35-465, 35-466, 35-467, 35-468, 35-469, 35-470, 35-471, 35-472, 35-473, 35-474, 35-475, 35-476, 35-477, 35-478, 35-479, 35-480, or 35-481 of SEQ ID NO:461, or to the corresponding Hyal-4 fragments of orthologs to these human Hyal-4 fragments.

In some embodiments, the hyaluronidase is Hyal-5 derived. Examples of such embodiments include polypeptides that have at least 80, 85, 90, 95, 96, 97, 98, 99, or 100% sequence identity to any of the Hyal-5 polypeptides provided herein, or to one or more of regions 1 to 8 (as provided herein) of a Hyal-5 polypeptide provided herein. HYAL5 is a pseudogene in humans and does not encode a functional protein in human. However, HYAL5 does encode functional enzymes in other species. Hyal-5 includes those of any origin including, cow (SEQ ID NO:470, 471, and 472) and mouse (SEQ ID NO:473). Typically, such Hya-5 derived polypeptide provided herein has hyaluronidase activity. In some embodiments, the hyaluronidase is a bovine Hyal-5 (bHyal-5). In some embodiments, the bHyal-5 has either an N-terminus deletion of amino acid residues 1-35, 1-36, 1-37, 1-38, 1-39, 1-40, 1-41, 1-42 or 1-43 of SEQ ID NO:470; and/or a C-terminus deletion of amino acid residues 457-553, 458-553, 459-553, 460-553, 461-553, 462-553, 463-553, 464-553, 465-553, 466-553, 467-553, 468-553, 469-553, 470-553, 471-553, 472-553, 473-553, 474-553, 475-553, 476-553, 477-553, 478-553, 479-553, 480-553, 481-553, 482-553, 483-553, 484-553, 485-553, 486-553, 487-553, 488-553, 489-553, 490-553, 491-553, 492-553, 493-553, 494-553, 495-553, 496-553, 497-553, 498-553, 499-553, 500-553, 501-553, 502-553, 503-553, 504-553, 505-553, 506-553, 507-553, 508-553, 509-553, 510-553, 511-553, 512-553, 513-553, 514-553, 515-553, 516-553, 517-553, 518-553, 519-553, 520-553, 521-553, 522-553, 523-553, 524-553, 525-553, 526-553, 527-553, 528-553, 529-553, 530-553, 531-553, 532-553, 533-553, 534-553, 535-553, 536-553, 537-553, 538-553, 539-553, 540-553, 541-553, 542-553, 543-553, 544-553, 545-553, 546-553, 547-553, 548-553, 549-553, 550-553, 551-553, 552-553, or 553 wherein the numbering is by reference to human bHyal-5 SEQ ID NO:470, and includes the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 36-457, 36-458, 36-459, 36-460, 36-461, 36-462, 36-463, 36-464, 36-465, 36-466, 36-467, 36-468, 36-469, 36-470, 36-471, 36-472, 36-473, 36-474, 36-475, 36-476, 36-477, 36-478, 36-479, 36-480, 36-481, 36-482, 36-483, 36-484, 36-485, 36-486, 36-487, 36-488, 36-489, 36-490, 36-491, 36-492, 36-493, 36-494, 36-495, 36-496, 36-497, 36-498, 36-499, 36-500, 36-501, 36-502, 36-503, 36-504, 36-505, 36-506, 36-507, 36-508, 36-509, 36-510, 36-511, 36-512, 36-513, 36-514, 36-515, 36-516, 36-517, 36-518, 36-519, 36-520, 36-521, 36-522, 36-523, 36-524, 36-525, 36-526, 36-527, 36-528, 36-529, 36-530, 36-531, 36-532, 36-533, 36-534, 36-535, 36-536, 36-537, 36-538, 36-539, 36-540, 36-541, 36-542, 36-543, 36-544, 36-545, 36-546, 36-547, 36-548, 36-549, 36-550, 36-551, 36-552, or 36-553 of SEQ ID NO:470, or to the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 36-457 of SEQ ID NO:470, or to the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 36-469 of SEQ ID NO:470, or to the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 39-457 of SEQ ID NO:470, or to the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 39-469 of SEQ ID NO:470, or to the corresponding Hyal-5 fragments of orthologs to these bovine Hyal-5 fragments. In some embodiments, the hyaluronidase fragment has at least 85%, 90%, 91%, 92%, 93%, 94%, 95%, 96%, 97%, 98%, 99% sequence identity to amino acid residues 39-472 of SEQ ID NO:470, or to the corresponding Hyal-5 fragments of orthologs to these bovine Hyal-5 fragments. In some embodiments, the hyaluronidase fragment has at least 95% sequence identity to amino acid residues 36-457, 36-458, 36-459, 36-460, 36-461, 36-462, 36-463, 36-464, 36-465, 36-466, 36-467, 36-468, 36-469, 36-470, 36-471, 36-472, 36-473, 36-474, 36-475, 36-476, 36-477, 36-478, 36-479, 36-480, 36-481, 36-482, 36-483, 36-484, 36-485, 36-486, 36-487, 36-488, 36-489, 36-490, 36-491, 36-492, 36-493, 36-494, 36-495, 36-496, 36-497, 36-498, 36-499, 36-500, 36-501, 36-502, 36-503, 36-504, 36-505, 36-506, 36-507, 36-508, 36-509, 36-510, 36-511, 36-512, 36-513, 36-514, 36-515, 36-516, 36-517, 36-518, 36-519, 36-520, 36-521, 36-522, 36-523, 36-524, 36-525, 36-526, 36-527, 36-528, 36-529, 36-530, 36-531, 36-532, 36-533, 36-534, 36-535, 36-536, 36-537, 36-538, 36-539, 36-540, 36-541, 36-542, 36-543, 36-544, 36-545, 36-546, 36-547, 36-548, 36-549, 36-550, 36-551, 36-552, or 36-553 of SEQ ID NO:470, or to the corresponding Hyal-5 fragments of orthologs to these bovine Hyal-5 fragments.

In some embodiments, the hyaluronidase is PH20 derived. Examples of such embodiments include polypeptides that have at least 80, 85, 90, 95, 96, 97, 98, 99, or 100% sequence identity to any of the HPH20 polypeptides provided herein, or to one or more of regions 1 to 8 (as provided herein) of a PH20 polypeptide provided herein. Typically, such PH20 derived polypeptide has hyaluronidase activity. PH20 includes those of any origin including, human, non-human primates, bovine, murine, rat, rabbit, or an ovine origin. Exemplary precursor PH20 polypeptides include those from human (SEQ ID NO:422), chimpanzee (SEQ ID NO:424), Rhesus monkey (SEQ ID NO:425), Cynomolgus monkey (SEQ ID NO:426), cow (SEQ ID NO:427), mouse (SEQ ID NO:428), rat (SEQ ID NO:429), rabbit (SEQ ID NO:430), and sheep (SEQ ID NOs:431, 432, and 433).

In some embodiments, the hyaluronidase is a PH20. In some embodiments, the PH20 has either an N-terminus deletion of amino acid residues 1-35, 1-36, 1-37, 1-38, 1-39, 1-40, 1-41, or 1-42 of SEQ ID NO:422, and/or a C-terminus deletion of amino acid residues 456-509, 457-509, 458-509, 459-509, 460-509, 461-509, 462-509, 463-509, 464-509, 465-509, 466-509, 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500 509, 501-509, 502-509, 503-509, 504-509, 505-509, 506-509, 507-509, 508-509, or 509 wherein the numbering is by reference to human PH20 SEQ ID NO:422, and includes the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 36-456, 36-457, 36-458, 36-459, 36-460, 36-461, 36-462, 36-463, 36-464, 36-465, 36-466, 36-467, 36-468, 36-469, 36-470, 36-471, 36-472, 36-473, 36-474, 36-475, 36-476, 36-477, 36-478, 36-479, 36-480, 36-481, 36-482, 36-483, 36-484, 36-485, 36-486, 36-487, 36-488, 36-489, 36-490, 36-491, 36-492, 36-493, 36-494, 36-495, 36-496, 36-497, 36-498, 36-499, 36-500, 36-501, 36-502, 36-503, 36-504, 36-505, 36-506, 36-507, 36-508, or 36-509 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 36-469 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 36-456 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 36-475 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 36-490 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 39-456 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 39-475 of SEQ ID NO:422, or to the corresponding PH20 fragments of orthologs to these human PH20 fragments. In some embodiments, the hyaluronidase fragment has at least 85%, 90%, 91%, 92%, 93%, 94%, 95%, 96%, 97%, 98%, 99% sequence identity to amino acid residues 39-490 of SEQ ID NO:422, or to the corresponding PH20 fragments of orthologs to these human PH20 fragments. In some embodiments, the hyaluronidase fragment has at least 95% sequence identity to amino acid residues 36-456, 36-457, 36-458, 36-459, 36-460, 36-461, 36-462, 36-463, 36-464, 36-465, 36-466, 36-467, 36-468, 36-469, 36-470, 36-471, 36-472, 36-473, 36-474, 36-475, 36-476, 36-477, 36-478, 36-479, 36-480, 36-481, 36-482, 36-483, 36-484, 36-485, 36-486, 36-487, 36-488, 36-489, 36-490, 36-491, 36-492, 36-493, 36-494, 36-495, 36-496, 36-497, 36-498, 36-499, 36-500, 36-501, 36-502, 36-503, 36-504, 36-505, 36-506, 36-507, 36-508, or 36-509 of NO:422, or to the corresponding PH20 fragments of orthologs to these human PH20 fragments.

In some embodiments, the ortholog is a bovine ortholog of the human PH20 fragments. In some embodiments, the bovine PH20 has either an N-terminus deletion of amino acid residues 1-33, 1-34, 1-35, 1-36, 1-37, 1-38, 1-39, or 1-40 of SEQ ID NO:427; and/or a C-terminus deletion of amino acid residues 467-509, 468-509, 469-509, 470-509, 471-509, 472-509, 473-509, 474-509, 475-509, 476-509, 477-509, 478-509, 479-509, 480-509, 481-509, 482-509, 483-509, 484-509, 485-509, 486-509, 487-509, 488-509, 489-509, 490-509, 491-509, 492-509, 493-509, 494-509, 495-509, 496-509, 497-509, 498-509, 499-509, 500 509, 501-509, 502-509, 503-509, 504-509, 505-509, 506-509, 507-509, 508-509, 510-509, 511-509, 512-509, 513-509, 514-509, 515-509, 516-509, 517-509, 518-509, 519-509, 520-509, 521-509, 522-509, 523-509, 524-509, 525-509, 526-509, 527-509, 528-509, 529-509, 530-509, 531-509, 532-509, 533-509, 534-509, 535-509, 536-509, 537-509, 538-509, 539-509, 540 509, 541-509, 542-509, 543-509, 544-509, 545-509, 546-509, 547-509, 548-509, 549-550, or 550 wherein the numbering is by reference to bovine bPH20 SEQ ID NO:427. In some embodiments, the hyaluronidase fragment has at least 85%, 90%, 91%, 92%, 93%, 94%, 95%, 96%, 97%, 98%, 99% sequence identity to amino acid residues 34-467, 34-468, 34-469, 34-470, 34-471, 34-472, 34-473, 34-474, 34-475, 34-476, 34-477, 34-478, 34-479, 34-480, 34-481, 34-482, 34-483, 34-484, 34-485, 34-486, 34-487, 34-488, 34-489, 34-490, 34-491, 34-492, 34-493, 34-494, 34-495, 34-496, 34-497, 34-498, 34-499, 34-500, 34-501, 34-502, 34-503, 34-504, 34-505, 34-506, 34-507, 34-508, 34-509, 34-510, 34-511, 34-512, 34-513, 34-514, 34-515, 34-516, 34-517, 34-518, 34-519, 34-520, 34-521, 34-522, 34-523, 34-524, 34-525, 34-526, 34-527, 34-528, 34-529, 34-530, 34-531, 34-532, 34-533, 34-534, 34-535, 34-536, 34-537, 34-538, 34-539, 34-540, 34-541, 34-542, 34-543, 34-544, 34-545, 34-546, 34-547, 34-548, 34-549, 34-550, 34-551, 34-552, 34-553, 34-554, or 34-505 of SEQ ID NO:427. In some embodiments, the hyaluronidase fragment has at least 85%, 90%, 91%, 92%, 93%, 94%, 95%, 96%, 97%, 98%, 99% sequence identity to amino acid residues 34-467 of SEQ ID NO:427. In some embodiments, the hyaluronidase fragment has at least 85%, 90%, 91%, 92%, 93%, 94%, 95%, 96%, 97%, 98%, 99% sequence identity to amino acid residues 34-473 of SEQ ID NO:427. In some embodiments, the hyaluronidase fragment has at least 85%, 90%, 91%, 92%, 93%, 94%, 95%, 96%, 97%, 98%, 99% sequence identity to amino acid residues 37-467 of SEQ ID NO:427. In some embodiments, the hyaluronidase fragment has at least 85%, 90%, 91%, 92%, 93%, 94%, 95%, 96%, 97%, 98%, 99% sequence identity to amino acid residues 37-473 of SEQ ID NO:427. In some embodiments, the hyaluronidase fragment has at least 95% sequence identity to amino acid residues 34-467, 34-468, 34-469, 34-470, 34-471, 34-472, 34-473, 34-474, 34-475, 34-476, 34-477, 34-478, 34-479, 34-480, 34-481, 34-482, 34-483, 34-484, 34-485, 34-486, 34-487, 34-488, 34-489, 34-490, 34-491, 34-492, 34-493, 34-494, 34-495, 34-496, 34-497, 34-498, 34-499, 34-500, 34-501, 34-502, 34-503, 34-504, 34-505, 34-506, 34-507, 34-508, 34-509, 34-510, 34-511, 34-512, 34-513, 34-514, 34-515, 34-516, 34-517, 34-518, 34-519, 34-520, 34-521, 34-522, 34-523, 34-524, 34-525, 34-526, 34-527, 34-528, 34-529, 34-530, 34-531, 34-532, 34-533, 34-534, 34-535, 34-536, 34-537, 34-538, 34-539, 34-540, 34-541, 34-542, 34-543, 34-544, 34-545, 34-546, 34-547, 34-548, 34-549, 34-550, 34-551, 34-552, 34-553, 34-554, or 34-505 of SEQ ID NO:427.

### Chimeras

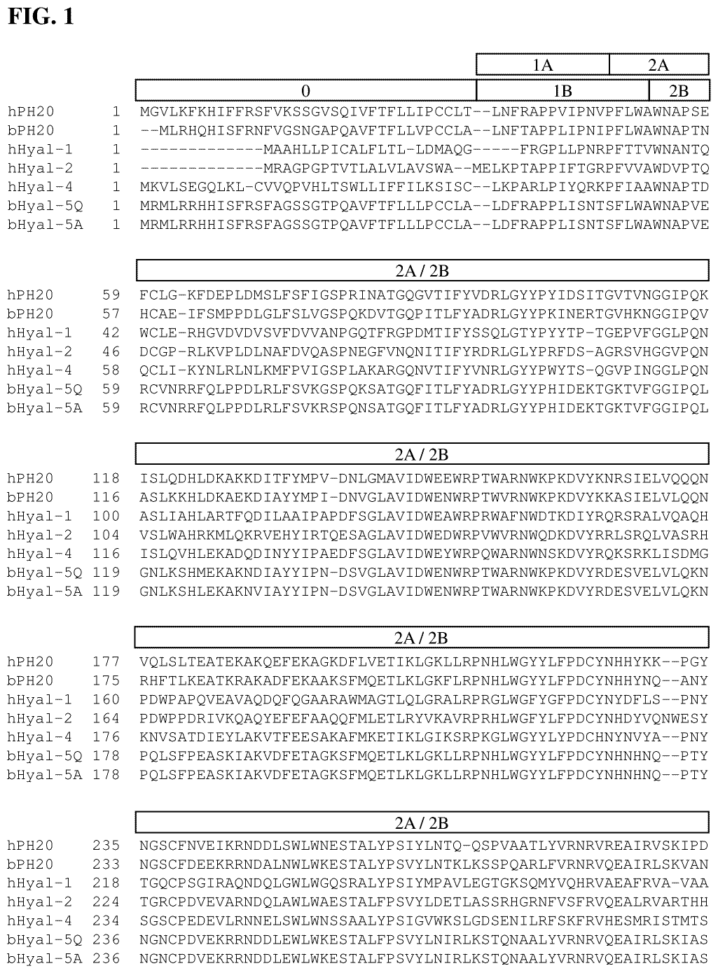

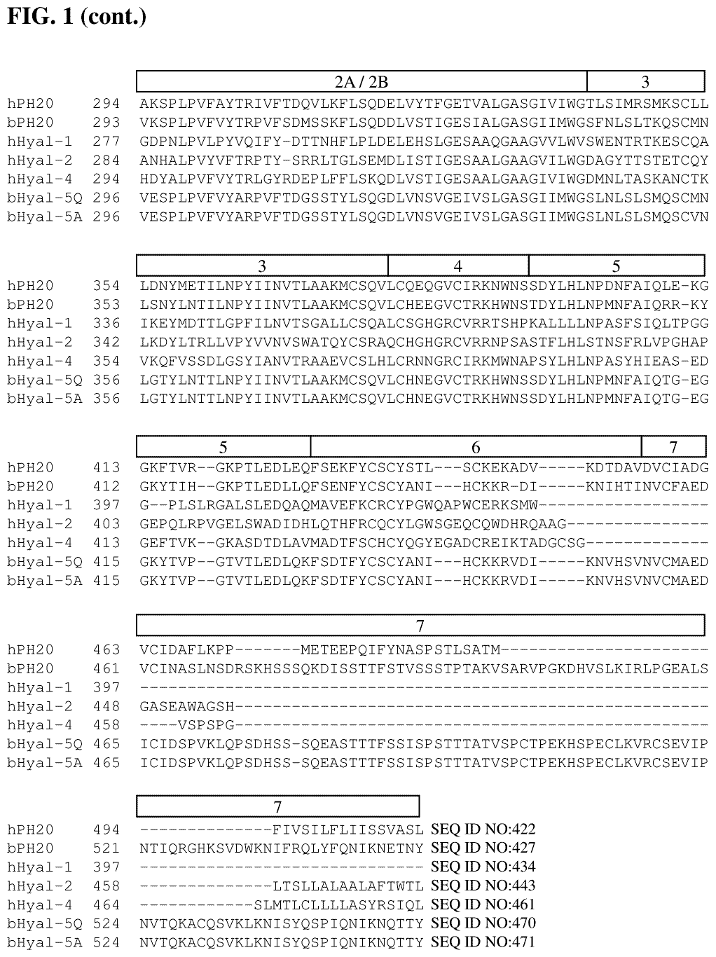

In some embodiments, the hyaluronidases are chimeric hyaluronidases. The amino acid sequences of human PH20 (“hPH20”) (Uniprot P38567, SEQ ID NO:422), bovine PH20 (“bPH20”) (Uniprot DA30460, SEQ ID NO:427), human Hyal-1 (“hHyal-1”) (Uniprot Q12794, SEQ ID NO:434), human Hyal-2 (hHyal-2) (Uniprot Q12891, SEQ ID NO:443), human Hyal-4 (“hHyal-4”) (Uniprot Q2M3T9, SEQ ID NO:461), bovine Hyal-5 (“bHyal-5Q”) (Uniprot Q2YDK3, SEQ ID NO:470), and bovine Hyal-5 (“bHyal-5A”) (Uniprot, AAV41022, SEQ ID NO:471) were aligned and divided into 8 Regions (Regions 0, 1, 2, 3, 4, 5, 6, and 7, each a “Region”) (FIG. 1). Two distinct cutoff points between Regions 1 and 2 resulted in two possible Regions 1 (Region 1A and 1B) and two possible Regions 2 (Regions 2A and 2B). As shown in FIG. 1, Region 1A pairs to Region 2A, and Region 1B pairs to Region 2B. Chimeric hyaluronidases can be generated by incorporating one or more Regions from one hyaluronidase with one or more Regions from at least one other hyaluronidase.

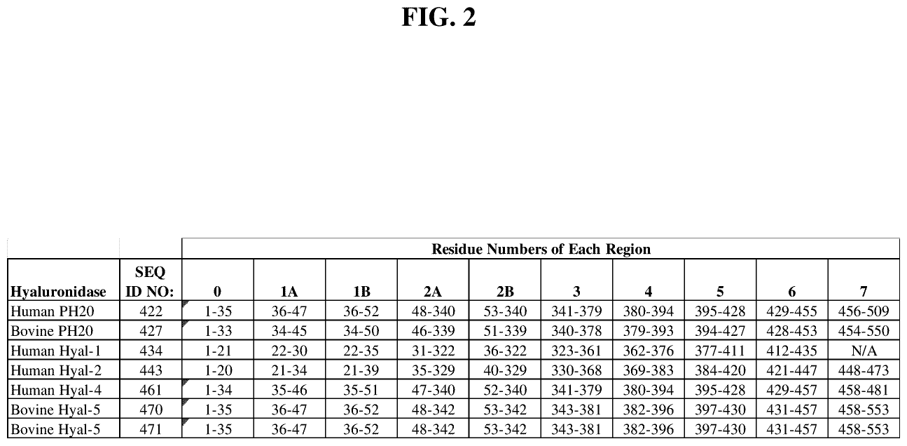

The amino acid positions corresponding to the Regions for each hyaluronidase are shown in FIG. 2. For hPH20 (SEQ ID NO:422), Region 0 corresponds to amino acids 1-35, Region 1A corresponds to amino acids 36-47, Region 1B corresponds to amino acids 36-52, Region 2A corresponds to amino acids 48-340, Region 2B corresponds to amino acids 53-340, Region 3 corresponds to amino acids 341-379, Region 4 corresponds to amino acids 380-394, Region 5 corresponds to amino acids 395-428, Region 6 corresponds to amino acids 429-455, and Region 7 corresponds to amino acids 456-509.

For bPH20 (SEQ ID NO:427), Region 0 corresponds to amino acids 1-33, Region 1A corresponds to amino acids 34-45, Region 1B corresponds to amino acids 34-50, Region 2A corresponds to amino acids 46-339, Region 2B corresponds to amino acids 51-339, Region 3 corresponds to amino acids 340-377, Region 4 corresponds to amino acids 378-393, Region 5 corresponds to amino acids 394-427, Region 6 corresponds to amino acids 428-453, and Region 7 corresponds to amino acids 454-550.

For hHyal-1 (SEQ ID NO:434), Region 0 corresponds to amino acids 1-21, Region 1A corresponds to amino acids 22-30, Region 1B corresponds to amino acids 22-35, Region 2A corresponds to amino acids 31-322, Region 2B corresponds to amino acids 36-322, Region 3 corresponds to amino acids 323-361, Region 4 corresponds to amino acids 362-376, Region 5 corresponds to amino acids 377-411, and Region 6 corresponds to amino acids 412-435. Hyal-1 does not comprise a Region 7.

For hHyal-2 (SEQ ID NO:443), Region 0 corresponds to amino acids 1-20, Region 1A corresponds to amino acids 21-34, Region 1B corresponds to amino acids 21-39, Region 2A corresponds to amino acids 35-329, Region 2B corresponds to amino acids 40-329, Region 3 corresponds to amino acids 330-368, Region 4 corresponds to amino acids 369-383, Region 5 corresponds to amino acids 384-420, Region 6 corresponds to amino acids 421-447, and Region 7 corresponds to amino acids 448-473.

For hHyal-4 (SEQ ID NO:461), Region 0 corresponds to amino acids 1-34, Region 1A corresponds to amino acids 35-46, Region 1B corresponds to amino acids 35-51, Region 2A corresponds to amino acids 47-340, Region 2B corresponds to amino acids 52-340, Region 3 corresponds to amino acids 341-379, Region 4 corresponds to amino acids 380-394, Region 5 corresponds to amino acids 395-428, Region 6 corresponds to amino acids 429-457, and Region 7 corresponds to amino acids 458-481.

For bHyal-5Q (SEQ ID NOs:470) and bHyal-5A (SEQ ID NO:471), Region 0 corresponds to amino acids 1-35, Region 1A corresponds to amino acids 36-47, Region 1B corresponds to amino acids 36-52, Region 2A corresponds to amino acids 48-342, Region 2B corresponds to amino acids 53-342, Region 3 corresponds to amino acids 343-381, Region 4 corresponds to amino acids 382-396, Region 5 corresponds to amino acids 397-430, Region 6 corresponds to amino acids 431-457, and Region 7 corresponds to amino acids 458-553.

In some embodiments, the chimeric hyaluronidase does not include a stretch of amino acids from Region 0 and/or Region 7, which correspond to a signal sequence and the carboxy terminus of a hyaluronidase, respectively. In some embodiments, the chimeric hyaluronidase comprises a stretch of amino acids from Regions 1, 2, 3, 4, 5, and 6, but lacks a stretch of amino acids from Regions 0 and 7. For example, a chimeric hyaluronidase can be generated that includes Regions 1A, 2A, 3, 4, and 6 of hPH20 and Region 5 of hHyal-4.

In some embodiments, the chimeric hyaluronidase comprises a stretch of amino acids from Regions 0, 1, 2, 3, 4, 5, and 6, but lacks a stretch of amino acids from Region 7. For example, a chimeric hyaluronidase can be generated that includes Regions 0, 1A, 2A, 3, 4, and 6 of hPH20 and Region 5 of hHyal-4.

In some embodiments, the chimeric hyaluronidase comprises a stretch of amino acids from Regions 1, 2, 3, 4, 5, 6 and 7, but lacks a stretch of amino acids from Region 0. For example, a chimeric hyaluronidase can be generated that includes Regions 1A, 2A, 3, 4, 6, and 7 of hPH20 and Region 5 of hHyal-4.

In some embodiments, the chimeric hyaluronidase comprises a stretch of amino acids from each of the 8 Regions. For example, a chimeric hyaluronidase can be generated that includes Regions 0, 1A, 2A, 3, 4, 6, and 7 of hPH20 and Region 5 of hHyal-4.

In some embodiments, the hyaluronidase includes a eukaryotic signal sequence to help direct expression of the hyaluronidase to the cell membrane. In any of the aspects and embodiments herein comprising a hyaluronidase, a hyaluronidase can include Region 0 from any hyaluronidase and typically includes one or more other regions from one or more other hyaluronidase and has hyaluronidase activity. In some embodiments, the hyaluronidase includes the Region 0 of SEQ ID NO:482 from hPH20. In some embodiments, the hyaluronidase includes the Region 0 of SEQ ID NO:492 from bPH20. In some embodiments, the hyaluronidase includes the Region 0 of SEQ ID NO:502 from hHyal-1. In some embodiments, the hyaluronidase includes the Region 0 of SEQ ID NO:511 from hHyal-2. In some embodiments, the hyaluronidase includes the Region 0 of SEQ ID NO:521 from hHyal-4. In some embodiments, the hyaluronidase includes the Region 0 of SEQ ID NO:531 from bHyal-5. In some embodiments of any of the hyaluronidases disclosed herein, the hyaluronidase does not include an amino acid sequence corresponding to Region 0, the signal sequence of a hyaluronidase, but typically includes one or more other regions from one or more other hyaluronidase, and has hyaluronidase activity. In some embodiments that lack a Region 0, the hyaluronidase can be linked to a heterologous signal sequence. The signal sequence can comprise any naturally occurring signal sequence or a synthetic signal sequence. In some embodiments, the signal sequence is a DAF signal sequence, a CD8 signal sequence, a human growth hormone signal sequence, tissue plaminogen activator signal sequence, chymotripsinogen signal sequence, human Ig kappa signal sequence, or a human serum albumin signal sequence. In some embodiments, the hyaluronidase does not include a signal sequence.

In any of the aspects and embodiments herein comprising a hyaluronidase, Region 1A of the hyaluronidase can be substituted with Region 1A from another hyaluronidase. In some embodiments, Region 1A of the hyaluronidase can be substituted with SEQ ID NO:483 from hPH20. In some embodiments, Region 1A of the hyaluronidase can be substituted with SEQ ID NO:493 from bPH20. In some embodiments, Region 1A of the hyaluronidase can be substituted with SEQ ID NO:503 from hHyal-1. In some embodiments, Region 1A of the hyaluronidase can be substituted with SEQ ID NO:512 from hHyal-2. In some embodiments, Region 1A of the hyaluronidase can be substituted with SEQ ID NO:522 from hHyal-4. In some embodiments, Region 1A of the hyaluronidase can be substituted with SEQ ID NO:532 from bHyal-5Q.

In any of the aspects and embodiments herein comprising a hyaluronidase, Region 1B of the hyaluronidase can be substituted with Region 1B from another hyaluronidase. In some embodiments, Region 1B of the hyaluronidase can be substituted with SEQ ID NO:484 from hPH20. In some embodiments, Region 1B of the hyaluronidase can be substituted with SEQ ID NO:494 from bPH20. In some embodiments, Region 1B of the hyaluronidase can be substituted with SEQ ID NO:504 from hHyal-1. In some embodiments, Region 1B of the hyaluronidase can be substituted with SEQ ID NO:513 from hHyal-2. In some embodiments, Region 1B of the hyaluronidase can be substituted with SEQ ID NO:523 from hHyal-4. In some embodiments, Region 1B of the hyaluronidase can be substituted with SEQ ID NO:533 from bHyal-5Q.

In any of the aspects and embodiments herein comprising a hyaluronidase, Region 1A or Region 1B of the hyaluronidase can be truncated at the amino terminus. In some embodiments, Region 1A or Region 1B of the hyaluronidase can be truncated at the amino terminus by 1, 2, 3, 4, 5, 6, 7, or 8 residues. In some embodiments, Region 1 of the hyaluronidase starts at residue 2, 3, 4, 5, 6, 7, 8, or 9 of Region 1A or Region 1B. In some embodiments, the amino acid sequence of the hyaluronidase starts with L36, N37, F38, R39, A40, P41, or P42 of hPH20 (SEQ ID NO:422). In some embodiments, the amino acid sequence of the hyaluronidase starts with L34, N35, F36, T37, A38, P39, or P40 of bPH20 (SEQ ID NO:427). In some embodiments, the amino acid sequence of the hyaluronidase starts with F22, R23, G24, or P25 of hHyal-1 (SEQ ID NO:434). In some embodiments, the amino acid sequence of the hyaluronidase starts with M21, E22, L23, K24, P25, T26, A27, P28, or P29 of hHyal-2 (SEQ ID NO:443). In some embodiments, the amino acid sequence of the hyaluronidase starts with L35, K36, P37, A38, R39, L40, or P41 of hHyal-4 (SEQ ID NO:461). In some embodiments, the amino acid sequence of the hyaluronidase starts with L36, D37, F38, R39, A40, P41, or P42 of bHyal-5 PH20 (SEQ ID NO:470). In some embodiments, the hyaluronidase is fused at the amino terminus to a heterologous protein other than to a hyaluronidase. In some embodiments, the hyaluronidase is fused at the amino terminus to an epitope tag.

In any of the aspects and embodiments herein comprising a hyaluronidase, Region 2A of the hyaluronidase can be substituted with Region 2A from another hyaluronidase. In some embodiments, Region 2A of the hyaluronidase can be substituted with SEQ ID NO:485 from hPH20. In some embodiments, Region 2A of the hyaluronidase can be substituted with SEQ ID NO:495 from bPH20. In some embodiments, Region 2A of the hyaluronidase can be substituted with SEQ ID NO:505 from hHyal-1. In some embodiments, Region 2A of the hyaluronidase can be substituted with SEQ ID NO:514 from hHyal-2. In some embodiments, Region 2A of the hyaluronidase can be substituted with SEQ ID NO:524 from hHyal-4. In some embodiments, Region 2A of the hyaluronidase can be substituted with SEQ ID NO:534 from bHyal-5Q. In some embodiments, Region 2A of the hyaluronidase can be substituted with SEQ ID NO:541 from bHyal-5A.

In any of the aspects and embodiments herein comprising a hyaluronidase, Region 2B of the hyaluronidase can be substituted with Region 2B from another hyaluronidase. In some embodiments, Region 2B of the hyaluronidase can be substituted with SEQ ID NO:486 from hPH20. In some embodiments, Region 2B of the hyaluronidase can be substituted with SEQ ID NO:496 from bPH20. In some embodiments, Region 2B of the hyaluronidase can be substituted with SEQ ID NO:506 from Hyal-1. In some embodiments, Region 2B of the hyaluronidase can be substituted with SEQ ID NO:515 from Hyal-2. In some embodiments, Region 2B of the hyaluronidase can be substituted with SEQ ID NO:525 from Hyal-4. In some embodiments, Region 2B of the hyaluronidase can be substituted with SEQ ID NO:535 from bHyal-5Q. In some embodiments, Region 2B of the hyaluronidase can be substituted with SEQ ID NO:542 from bHyal-5A.

In any of the aspects and embodiments herein comprising a hyaluronidase, Region 3 of the hyaluronidase can be substituted with Region 3 from another hyaluronidase. In some embodiments, Region 3 of the hyaluronidase can be substituted with SEQ ID NO:487 from hPH20. In some embodiments, Region 3 of the hyaluronidase can be substituted with SEQ ID NO:497 from bPH20. In some embodiments, Region 3 of the hyaluronidase can be substituted with SEQ ID NO:507 from hHyal-1. In some embodiments, Region 3 of the hyaluronidase can be substituted with SEQ ID NO:516 from hHyal-2. In some embodiments, Region 3 of the hyaluronidase can be substituted with SEQ ID NO:526 from hHyal-4. In some embodiments, Region 3 of the hyaluronidase can be substituted with SEQ ID NO:536 from bHyal-5Q. In some embodiments, Region 3 of the hyaluronidase can be substituted with SEQ ID NO:543 from bHyal-5A.

In any of the aspects and embodiments herein comprising a hyaluronidase, Region 4 of the hyaluronidase can be substituted with Region 4 from another hyaluronidase. In some embodiments, Region 4 of the hyaluronidase can be substituted with SEQ ID NO:488 from hPH20. In some embodiments, Region 4 of the hyaluronidase can be substituted with SEQ ID NO:498 from bPH20. In some embodiments, Region 4 of the hyaluronidase can be substituted with SEQ ID NO:508 from hHyal-1. In some embodiments, Region 4 of the hyaluronidase can be substituted with SEQ ID NO:517 from hHyal-2. In some embodiments, Region 4 of the hyaluronidase can be substituted with SEQ ID NO:527 from hHyal-4. In some embodiments, Region 4 of the hyaluronidase can be substituted with SEQ ID NO:537 from bHyal-5Q.

In any of the aspects and embodiments herein comprising a hyaluronidase, Region 5 of the hyaluronidase can be substituted with Region 5 from another hyaluronidase. In some embodiments, Region 5 of the hyaluronidase can be substituted with SEQ ID NO:489 from hPH20. In some embodiments, Region 5 of the hyaluronidase can be substituted with SEQ ID NO:499 from bPH20. In some embodiments, Region 5 of the hyaluronidase can be substituted with SEQ ID NO:509 from hHyal-1. In some embodiments, Region 5 of the hyaluronidase can be substituted with SEQ ID NO:518 from hHyal-2. In some embodiments, Region 5 of the hyaluronidase can be substituted with SEQ ID NO:528 from hHyal-4. In some embodiments, Region 5 of the hyaluronidase can be substituted with SEQ ID NO:538 from bHyal-5Q.

In any of the aspects and embodiments herein comprising a hyaluronidase, Region 6 of the hyaluronidase can be substituted with Region 6 from another hyaluronidase. In some embodiments, Region 6 of the hyaluronidase can be substituted with SEQ ID NO:490 from hPH20. In some embodiments, Region 6 of the hyaluronidase can be substituted with SEQ ID NO:500 from bPH20. In some embodiments, Region 6 of the hyaluronidase can be substituted with SEQ ID NO:510 from hHyal-1. In some embodiments, Region 6 of the hyaluronidase can be substituted with SEQ ID NO:519 from hHyal-2. In some embodiments, Region 6 of the hyaluronidase can be substituted with SEQ ID NO:529 from hHyal-4. In some embodiments, Region 6 of the hyaluronidase can be substituted with SEQ ID NO:539 from bHyal-5Q.

In any of the aspects and embodiments herein comprising a hyaluronidase, the hyaluronidase can include a Region 7. In any of the aspects and embodiments herein comprising a hyaluronidase that includes a Region 7, Region 7 of the hyaluronidase can be substituted with Region 7 from another hyaluronidase. In some embodiments, Region 7 of the hyaluronidase can be substituted with SEQ ID NO:491 from hPH20. In some embodiments, Region 7 of the hyaluronidase can be substituted with SEQ ID NO:501 from bPH20. In some embodiments, Region 7 of the hyaluronidase can be substituted with SEQ ID NO:520 from hHyal-2. In some embodiments, Region 7 of the hyaluronidase can be substituted with SEQ ID NO:530 from hHyal-4. In some embodiments, Region 7 of the hyaluronidase can be substituted with SEQ ID NO:540 from bHyal-5Q.

In any of the aspects and embodiments herein comprising a hyaluronidase, Region 7 of the hyaluronidase can be truncated at any residue. In some embodiments, Region 7 of the hyaluronidase can be truncated after residue 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, or 83 of Region 7, depending on the length of Region 7 present in the hyaluronidase.

In some embodiments, the hyaluronidase can have or include any one of the sequences of SEQ ID NOs:544-1003, or a sequence that is at least 80, 85, 90, 95, 96, 97, 98, or 99% identical to SEQ ID NOs:544-1003. In some embodiments, the hyaluronidase can include a sequence with at least 80% sequence identity to any one of the sequences of SEQ ID NOs:544-1003. In some embodiments, the hyaluronidase can include a sequence with at least 85% sequence identity to any one of the sequences of SEQ ID NOs:544-1003. In some embodiments, the hyaluronidase can include a sequence with at least 90% sequence identity to any one of the sequences of SEQ ID NOs:544-1003. In some embodiments, the hyaluronidase can include a sequence with at least 95% sequence identity to any one of the sequences of SEQ ID NOs:544-1003. In some embodiments, the hyaluronidase can include a sequence with at least 96% sequence identity to any one of the sequences of SEQ ID NOs:544-1003. In some embodiments, the hyaluronidase can include a sequence with at least 97% sequence identity to any one of the sequences of SEQ ID NOs:544-1003. In some embodiments, the hyaluronidase can include a sequence with at least 98% sequence identity to any one of the sequences of SEQ ID NOs:544-1003. In some embodiments, the hyaluronidase can include a sequence with at least 99% sequence identity to any one of the sequences of SEQ ID NOs:544-1003.

In some embodiments, the chimeric hyaluronidase is not a chimera of human PH20 and human Hyal-1. In some embodiments, the chimeric hyaluronidase is not a chimeric hyaluronidase comprising Regions 2A, 3, 4, 5, and 6 of hPH20 and Region 1A of hHyal-1. In some embodiments, the chimeric hyaluronidase is not a chimeric hyaluronidase comprising Regions 2B, 3, 4, 5, and 6 of hPH20 and Region 1B of hHyal-1. In some embodiments, the chimeric hyaluronidase is not a chimeric hyaluronidase comprising Regions 1B, 2B, 4, 5, and 6 of hPH20 and Region 3 of hHyal-1. In some embodiments, the chimeric hyaluronidase is not a chimeric hyaluronidase comprising Regions 2B, 4, 5, and 6 of hPH20 and Regions 1B and 3 of hHyal-1.

In some embodiments, the chimeric hyaluronidase comprises a sequence with at least 95% identity to a sequence comprised of any combination of Regions 1B, 2B, 3, 4, 5, and 6 from 2 or more hyaluronidases selected from hPH20 (SEQ ID NO:422), bPH20 (SEQ ID NO:427), hHyal-1 (SEQ ID NO:434), hHyal-2 (SEQ ID NO:443), hHyal-4 (SEQ ID NO:461), bHyal-5 (SEQ ID NO:470), and bHyal-5 (SEQ ID NO:471), and optionally wherein the chimeric hyaluronidase does not comprise Regions 2B, 4, 5, and 6 of hPH20 and Regions 1B and 3 of hHyal-1. In some embodiments, the chimeric hyaluronidase comprises any combination of Regions 1B, 2B, 3, 4, 5, and 6 from 2 or more hyaluronidases selected from hPH20 (SEQ ID NO:422), bPH20 (SEQ ID NO:427), hHyal-1 (SEQ ID NO:434), hHyal-2 (SEQ ID NO:443), hHyal-4 (SEQ ID NO:461), b Hyal-5 (SEQ ID NO:470), and bHyal-5 (SEQ ID NO:471), and optionally wherein the chimeric hyaluronidase does not comprise Regions 2B, 4, 5, and 6 of hPH20 and Regions 1B and 3 of hHyal-1.

In some embodiments, the hyaluronidase does not comprise an amino acid substitution at a position corresponding to 341 of hPH20 (SEQ ID NO:422) or at the corresponding position of any of the hyaluronidases disclosed herein. In some embodiments, the hyaluronidase does not comprise the amino acid substitution corresponding to T341A of hPH20 (SEQ ID NO:422) or at the corresponding position of any of the hyaluronidases disclosed herein. In some embodiments, the hyaluronidase does not comprise the amino acid substitution corresponding to T341C of hPH20 (SEQ ID NO:422) or at the corresponding position of any of the hyaluronidases disclosed herein. In some embodiments, the hyaluronidase does not comprise the amino acid substitution corresponding to T341D of hPH20 (SEQ ID NO:422) or at the corresponding position of any of the hyaluronidases disclosed herein. In some embodiments, the hyaluronidase does not comprise the amino acid substitution corresponding to T341G of hPH20 (SEQ ID NO:422) or at the corresponding position of any of the hyaluronidases disclosed herein. In some embodiments, the hyaluronidase does not comprise the amino acid substitution corresponding to T341S of hPH20 (SEQ ID NO:422) or at the corresponding position of any of the hyaluronidases disclosed herein.

In some embodiments, the hyaluronidase is a hyaluronidase glycoprotein. In some embodiments, 1 or more Asparagine residue of the hyaluronidase that would otherwise become glycosylated, is deleted or mutated to another amino acid such as Alanine. In some embodiments, 2 or more Asparagine residues of the hyaluronidase that could otherwise become glycosylated, is deleted or mutated to another amino acid such as Alanine. In some embodiments, 3 or more Asparagine residue of the hyaluronidase that could otherwise become glycosylated, are deleted or mutated to another amino acid such as Alanine. Asparagine residues in human PH20 that are capable of being glycosylated by post-translation modification include amino acids N82, N166, N235, N254, N368, and N393 of (SEQ ID NO:422). In some embodiments, 1, 2, 3, 4, 5, or all of the Asparagine residues of a PH20 variant at positions corresponding to amino acids 82, 166, 254, 368, and 393 of SEQ ID NO:422 are deleted or mutated to another amino acid. In some embodiments, 1, 2, or all 3 of the Asparagine residues of a bPH20 variant at positions corresponding to amino acids 233, 367, and 392 of SEQ ID NO:427 are deleted or mutated to another amino acid. In some embodiments, 1, 2, or all 3 of the Asparagine residues of a bHyal5 variant at positions corresponding to amino acids 236, 370, and 395 of SEQ ID NO:470 are deleted or mutated to another amino acid.

### Membrane-Bound Hyaluronidases

Membrane-bound hyaluronidases are included in methods and compositions disclosed herein, and are also an aspect of the invention. In some embodiments, the membrane-bound hyaluronidase is a polypeptide associated with and/or on the outer membrane or surface of a virus such as a RIP, on the plasma membrane of a cell line such as a packing cell, or on the plasma membrane of a primary cell such as a primary T cell, NK cell, macrophage, epithelial cell, neuron, or other modified, genetically modified, and/or transduced cell described herein.

Some hyaluronidases are naturally membrane-associated. For example, hPH20, bPH20, hHyal-2, hHyal-4, and bHyal-5 can be naturally membrane associated through a GPI linkage. In some embodiments, the hyaluronidase may be membrane-bound via their endogenous GPI anchors. In some embodiments, the membrane-bound hyaluronidase is hPH20 (SEQ ID NO:422), bPH20 (SEQ ID NO:427), hHyal-2 (SEQ ID NO:443), hHyal-4 (SEQ ID NO:461), b Hyal-5Q (SEQ ID NO:470), or bHyal-5A (SEQ ID NO:471). In some embodiments, the membrane-bound hyaluronidase is a variant of hPH20 (SEQ ID NO:422), bPH20 (SEQ ID NO:427), hHyal-2 (SEQ ID NO:443), hHyal-4 (SEQ ID NO:461), or b Hyal-5Q (SEQ ID NO:470) that comprises the membrane attachment sequence. In some embodiments, the membrane-bound hyaluronidase is a variant of hPH20 (SEQ ID NO:422), bPH20 (SEQ ID NO:427), hHyal-2 (SEQ ID NO:443), hHyal-4 (SEQ ID NO:461), b Hyal-5 (SEQ ID NO:470), or bHyal-5 (SEQ ID NO:471) that comprises a membrane attachment sequence that has at least 95% sequence identity to Region 7 from any of hPH20 (SEQ ID NO:491), bPH20 (SEQ ID NO:501), hHyal-2 (SEQ ID NO:520, hHyal-4 (SEQ ID NO:530), bHyal-5 (SEQ ID NO:540), or an ortholog of any of the foregoing. In some embodiments, the GPI attachment site is mutated to reduce shedding of the GPI-linked protein from the membrane.

In some embodiments, the hyaluronidase may be membrane-bound via a heterologous membrane attachment sequence that is fused to the C-terminus of the hyaluronidase. In some embodiments the heterologous membrane attachment sequence is a GPI anchor. In some embodiments, the heterologous membrane attachment sequence is a transmembrane domain. In some embodiments the heterologous membrane attachment sequence is fused to a hyaluronidase that is truncated including to remove any endogenous membrane attachment sequence. In some embodiments, the membrane-associated hyaluronidase is a chimeric hyaluronidase. In any embodiment comprising a heterologous membrane attachment sequence, an optional amino acid linker may connect the hyaluronidase to the membrane attachment sequence.

In some embodiments, the heterologous membrane attachment sequence is a GPI anchor attachment sequence. The heterologous GPI anchor attachment sequence can be derived from any known GPI-anchored protein (reviewed in Ferguson MAJ, Kinoshita T, Hart G W. Glycosylphosphatidylinositol Anchors. In: Varki A, Cummings R D, Esko J D, et al., editors. Essentials of Glycobiology. 2nd edition. Cold Spring Harbor (NY): Cold Spring Harbor Laboratory Press; 2009. Chapter 11). In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In an illustrative embodiment, the heterologous GPI anchor attachment sequence is derived from Fc receptor FcγRIIIb (CD16b). In some embodiments, the GPI anchor is the GPI anchor of DAF.

In some embodiments, the heterologous membrane attachment sequence is a transmembrane domain. The transmembrane domain may be any transmembrane domain disclosed herein for insertion into a eukaryotic cell membrane, including those transmembrane domains associated with engineered signaling polypeptides. In some embodiments, the transmembrane domains consists of short carboxy termini. Short carboxy termini minimize interference with the intracellular viral matrix proteins. In some embodiments, the transmembrane domain is from GP120, or any other transmembrane protein that interacts favorably with viral matrix proteins. In some embodiments, the transmembrane domain is from PDGFR. In illustrative embodiments, the transmembrane domain naturally accumulates in lipid rafts.

In some embodiments, the hyaluronidase is soluble. In some embodiments, the hyaluronidase is shed from the membrane. In some embodiments, the hyaluronidase has the property of being, and/or is designed to be shed from the membrane in an in vivo environment, or lacks a sequence for remaining bound to a membrane. In some embodiments, the soluble hyaluronidase lacks a membrane attachment sequence. In some embodiments, the soluble hyaluronidase is truncated to remove the endogenous membrane attachment sequence. In some embodiments the soluble hyaluronidase is a chimeric hyaluronidase that is not fused to a membrane attachment sequence. In some embodiments, the soluble hyaluronidase is truncated at amino acid position A467, E477, P478, Q479, 1480, N483 of hPH20 (SEQ ID NO:422) or at the corresponding position in a chimeric hyaluronidase or in a paralog or ortholog of hPH20.

Some embodiments include polynucleotides that encode any of the hyaluronidases disclosed herein, including fusions to heterologous signal sequences and/or membrane attachment sequences. SEQ ID NOs:1004-1018 are each representative polynucleotides encoding the human trypsinogen signal sequence fused to a soluble chimeric hyaluronidases. SEQ ID NOs:1019-1033 are each representative polynucleotides encoding the human trypsinogen signal sequence fused to a chimeric hyaluronidases with a GPI attachment sequence from CD16b.

In some embodiments, the polynucleotide is within an expression vector such as one that could be used to transfect a cell line such as a packaging cell. In some embodiments, the polynucleotide encoding the hyaluronidase remains extrachromosomal in the cell line. Thus, in some embodiments, cells are provided herein that include an extrachromosomal polynucleotide encoding a hyaluronidase polypeptide herein. In some embodiments, the polynculeotide encoding the hyaluronidase is stably integrated into the genome of the cell lines. In some embodiments, the polynucleotide encoding the hyaluronidase is in the genome of a virus such as a RIP, or a population thereof. In some embodiments, the polynucleotide encoding the hyaluronidase is within a primary cell such as a primary T cell, NK cell, macrophage, epithelial cell, neuron, or other modified cell described herein, or a population thereof. In some embodiments, the polynucleotide encoding the hyaluronidase remains extrachromosomal in the primary cell. In some embodiments, the polynucleotide encoding the hyaluronidase is stably integrated into the genome of the primary cell lines.

In illustrative embodiments, the membrane-associated hyaluronidase is on the surface of a virus, such as a RIP. The viral packaging cell may express the hyaluronidase protein on its cell membrane. During viral packaging, portions of the cell membrane that comprise the hyaluronidase will be incorporated into the virus membrane.

In some embodiments, the hyaluronidase is expressed on the surface of the cell that is modified, genetically modified, and/or transduced by the virus comprising a hyaluronidase. During fusion, portions of the viral membrane that comprise the hyaluronidase will be incorporated into the cell membrane. In some embodiments, genetically modified and/or transduced cell may express the hyaluronidase protein on its cell membrane.

### Hyaluronidase Activity

The hyaluronidase activity of a polypeptide, whether soluble or immobilized (e.g. membrane bound) on a retroviral particle, may be assessed using any one or more of several methods well known in the art. For example, the USP XXII assay for hyaluronidase determines activity indirectly by measuring the amount of undegraded hyaluronic acid, or hyaluronan, (HA) substrate remaining after incubation with retroviral particle immobilized or free enzyme with the HA at 37° C. (USP XXII-NFXVII (1990) 644-645 United States Pharmacopeia Convention, Inc, Rockville, Md.). A Hyaluronidase Reference Standard (USP) or National Formulary (NF) Standard Hyaluronidase solution can be used in an assay to ascertain the activity, in Units, of any hyaluronidase. Other standards may include commercially available recombinant human hyaluronidase (Hylenex®) or ovine testes-derived hyaluronidase (Vitrase®). Alternative methods for measuring hyaluronidase enzymatic activity include a microturbidity assay, and substrate gel zymography as described in U.S. Pat. No. 9,284,543, incorporated by reference herein in its entirety. Additionally, purified high molecular weight hyaluronan substrate may be incubated with the recombinant on-test polypeptide in either soluble or immobilized (e.g. membrane bound on retroviral particles) form followed by acrylamide electrophoresis with alcian blue staining for characterizing the depolymerized substrate. In each assay, the pH of the enzymatic assay reaction solution should be greater than or equal to pH 7.0 to establish the neutral dependent enzymatic activity relative to the Reference Standard. Comparison at other pH may be used to evaluate the relative potency of the enzyme by pH. In some embodiments the hyaluronidase has an activity of at least 2,000 USP Units/mg protein. In some embodiments of the invention, the hyaluronidase has at least 30% of the hyaluronidase activity of a polypeptide with amino acids 36-482 of SEQ ID NO:420. In some embodiments, the immobilized retroviral particle contains from 1-150 USP Units of hyaluronidase per microgram of P24 protein.

### Absolute Numbers of Hyaluronidases on the Surface of a Viral Particle

The absolute number of individual hyaluronidase polypeptides on the surface of a viral particle, and in illustrative embodiments a retroviral particle, can affect how the viral particle degrades the extracellular matrix (ECM). Not to be limited by theory, higher absolute numbers of hyaluronidase polypeptides on the surface of a viral particle can increase the degradation of the ECM and allow the viral particle to move through the ECM more easily. Depending on the target cell and location, fewer or more hyaluronidase polypeptides on the surface of the viral particle may be required. Thus, the absolute number of hyaluronidase polypeptides on the surface of a viral particle can help determine the binding of the targeting element.

In some embodiments, each viral particle, and in illustrative embodiments a retroviral particle, can include at least 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle. In some embodiments, each viral particle can include no more than 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, or 4,500 hyaluronidase polypeptides on the surface of one retroviral particle on the low end of the range and 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle on the high end of the range.

In some embodiments, between 100 hyaluronidase polypeptides on the surface of one retroviral particle on the low end of the range and 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle on the high end of the range. In some embodiments, between 500 hyaluronidase polypeptides on the surface of one retroviral particle on the low end of the range and 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle on the high end of the range. In some embodiments, between 1,000 hyaluronidase polypeptides on the surface of one retroviral particle on the low end of the range and 1,100, 1,200, 1,300, 1,400, 1,500, 1,600, 1,700, 1,800, 1,900, 2,000, 2,500, 3,000, 3,500, 4,000, 4,500, or 5,000 hyaluronidase polypeptides on the surface of one retroviral particle on the high end of the range. In some embodiments, between 2,000 hyaluronidase polypeptides on the surface of one retroviral particle on the low end of the range and 2,500, 3,000, 3,500, 4,000, 4,500, or 5,000 hyaluronidase polypeptides on the surface of one retroviral particle on the high end of the range.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, or 4,500 hyaluronidase polypeptides on the surface of one retroviral particle on the low end of the range and 5,000 hyaluronidase polypeptides on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, or 3,500 hyaluronidase polypeptides on the surface of one retroviral particle on the low end of the range and 4,000 hyaluronidase polypeptides on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, or 2,500 hyaluronidase polypeptides on the surface of one retroviral particle on the low end of the range and 3,000 hyaluronidase polypeptides on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, or 1,900 hyaluronidase polypeptides on the surface of one retroviral particle on the low end of the range and 2,000 hyaluronidase polypeptides on the surface of one retroviral particle on the high end of the range.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, or 900 hyaluronidase polypeptides on the surface of one retroviral particle on the low end of the range and 1,000 hyaluronidase polypeptides on the surface of one retroviral particle on the high end of the range.

In some embodiments, each viral particle can include between 50 and 5,000 hyaluronidase polypeptides on the surface of one viral particle. In some embodiments, each viral particle can include between 500 and 4,500 hyaluronidase polypeptides on the surface of one viral particle. In some embodiments, each viral particle can include between 2,000 and 4,000 hyaluronidase polypeptides on the surface of one viral particle. In some embodiments, each viral particle can include between 2,500 and 3,500 hyaluronidase polypeptides on the surface of one viral particle. In some embodiments, each viral particle can include between 250 and 1,000 hyaluronidase polypeptides on the surface of one viral particle. In some embodiments, each viral particle can include between 500 and 750 hyaluronidase polypeptides on the surface of one viral particle.

### Isolated Polypeptides

Hyaluronidase polypeptides disclosed herein, include chimeric hyaluronidase polypeptides that include various combinations of structural domains from different hyaluronidase polypeptides. The hyaluronidase polypeptides disclosed herein are themselves aspects of the present disclosure. Thus, in one aspect provided herein is an isolated (e.g., substantially purified) polypeptide comprising an amino acid sequence with at least 80%, 85%, 90%, 95%, 96%, 97%, 98%, or 99% sequence identity or identical to any one of SEQ ID NOs:544-1003. Typically, such polypeptides have hyaluronidase activity. In some embodiments, the isolated polypeptide comprises an amino acid sequence with at least 90% sequence identity to any one of SEQ ID NOs:544-1003. In some embodiments, the isolated polypeptide comprises an amino acid sequence with at least 95% sequence identity to any one of SEQ ID NOs:544-1003. In some embodiments, the isolated polypeptide comprises an amino acid sequence with at least 96% sequence identity to any one of SEQ ID NOs:544-1003. In some embodiments, the isolated polypeptide comprises an amino acid sequence with at least 97% sequence identity to any one of SEQ ID NOs:544-1003. In some embodiments, the isolated polypeptide comprises an amino acid sequence with at least 98% sequence identity to any one of SEQ ID NOs:544-1003. In some embodiments, the isolated polypeptide comprises an amino acid sequence with at least 99% sequence identity to any one of SEQ ID NOs: CH1-CH460. In some embodiments, the isolated polypeptide comprises an amino acid sequence of any one of SEQ ID NOs:544-1003.

Isolated cells, polypeptides or polynucleotides, (or nucleic acids/nucleic acid sequences) disclosed herein are typically, in some embodiments, substantially free of components normally associated in their naturally occurring or native state, or due to construction or processing, such as cellular material, gel materials, culture medium, and contaminating polypeptides or nucleic acids (such as from nucleic acid libraries or expression products therefrom), except as described herein, when produced by recombinant techniques; or substantially free of chemical precursors or other chemicals when chemically synthesized, except as described herein. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In some embodiments, the cells are cultured in vitro. In other embodiments, the cells are not cultured in vitro.

In aspects and embodiments herein, a substantially purified polypeptide can include a polypeptide that can be substantially or essentially free of components that normally accompany or interact with the polypeptide as found in its naturally occurring environment, i.e., a native cell, or host cell in the case of recombinantly produced polypeptides or polypeptide variants that may be substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein. When polypeptide or variant thereof is recombinantly produced by the host cells, the protein may be present at about 30%, about 25%, about 20%, about 15%, about 10%, about 5%, about 4%, about 3%, about 2%, or about 1% or less of the dry weight of the cells. When a polypeptide or variant thereof is recombinantly produced by the host cells, the protein may be present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. Thus, “substantially purified” polypeptide or polypeptide variant as produced by the methods of the present invention may have a purity level of at least about 30%, at least about 35%, at least about 40%, at least about 45%, at least about 50%, at least about 55%, at least about 60%, at 19 least about 65%, at least about 70%, specifically, a purity level of at least about 75%, 80%, 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

### Isolated Polynucleotides

Polynucleotides encoding the hyaluronidase polypeptides disclosed herein are themselves aspects of the present disclosure. Thus, in one aspect provided herein is an isolated (e.g., substantially purified polynucleotide) comprising a nucleic acid sequence encoding an amino acid sequence with at least 80%, 85%, 90%, 95%, 96%, 97%, 98%, or 99% sequence identity or identical to any one of SEQ ID NOs: CH1-CH460. In some embodiments, the isolated polynucleotide comprises a nucleic acid sequence encoding an amino acid sequence with at least 90% sequence identity to any one of SEQ ID NOs: CH1-CH460. In some embodiments, the isolated polynucleotide comprises a nucleic acid sequence encoding an amino acid sequence with at least 95% sequence identity to any one of SEQ ID NOs: CH1-CH460. In some embodiments, the isolated polynucleotide comprises a nucleic acid sequence encoding an amino acid sequence with at least 96% sequence identity to any one of SEQ ID NOs: CH1-CH460. In some embodiments, the isolated polynucleotide comprises a nucleic acid sequence encoding an amino acid sequence with at least 97% sequence identity to any one of SEQ ID NOs: CH1-CH460. In some embodiments, the isolated polynucleotide comprises a nucleic acid sequence encoding an amino acid sequence with at least 98% sequence identity to any one of SEQ ID NOs: CH1-CH460. In some embodiments, the isolated polynucleotide comprises a nucleic acid sequence encoding an amino acid sequence with at least 99% sequence identity to any one of SEQ ID NOs: CH1-CH460. In some embodiments, the isolated polynucleotide comprises a nucleic acid sequence encoding an amino acid sequence of any one of SEQ ID NOs: CH1-CH460.

### Rip Formulations, Cell Formulations and Methods of Use Thereof

Recombinant viral particle formulations are disclosed in methods and compositions provided herein, that typically include a plurality of recombinant viral particles having a membrane-bound hyaluronidase polypeptide. Such formulations can be used for example, to modify cells, as non-limiting examples human cells, primary cells, T cells and/or NK cells to make genetically modified and/or transduced cells, human cells, primary cells, T cells and/or NK cells, in ex vivo, in vitro, or in vivo methods, for example that can include administration of the formulation to a subject. The recombinant viral particles, and formulations thereof, are themselves aspects of the present disclosure. Typically, the recombinant viral particles included in aspects provided herein, are recombinant retroviral particles, and in some embodiments are replication incompetent, meaning that a recombinant retroviral particle cannot replicate once it leaves the packaging cell, and thus cannot replicate in a subject, for example when administered to the subject. In fact, unless indicated otherwise herein, retroviral particles are replication incompetent, and if such retroviral particles include nucleic acids in their genome that are not native to the retrovirus, they are “recombinant retroviral particles.” In illustrative embodiments, the recombinant retroviral particles are lentiviral particles. Replication incompetent recombinant retroviral particles are referred to herein as RIPs and formulations that include RIPs, typically a plurality of RIPs, can be referred to as RIP formulations. A skilled artisan can understand how the various aspects and embodiments disclosed herein could be modified for other viral and retroviral particles, or for non-viral recombinant vectors. In some embodiments, a RIP formulation includes a RIP, or a plurality thereof, with a membrane-bound hyaluronidase. In some embodiments, a RIP formulation can include a RIP with a polynucleotide encoding a membrane-bound hyaluronidase or a soluble hyaluronidase. Cell formulations are provided herein that include for example T cells and/or NK cells. Such formulations, in illustrative embodiments are provided by methods provided herein. In some embodiments, the cell formulation can include a T and/or NK cell with any of the membrane-bound hyaluronidases disclosed elsewhere herein. In some embodiments, the cell formulation can include any of the soluble hyaluronidases disclosed elsewhere herein. In some embodiments, the cell formulation can include a T and/or NK cell with a polynucleotide encoding any of the hyaluronidases disclosed elsewhere herein. In some embodiments, the hyaluronidases are membrane-bound. In some embodiments, the hyaluronidase are soluble.

In some embodiments, the delivery solution, RIP formulation, or cell formulation is compatible with, effective for or even adapted for perilymphatic, subcutaneous, or intramuscular delivery A skilled artisan can understand how the various aspects and embodiments disclosed herein could be modified for other delivery types, for example, for, intratumoral, intraocular, intraperitoneal, intravenous, intravesical infusion, intravitreal, intrathecal, intrathecal, or intra-arterial delivery. For example, the concentration of unmodified or modified cells in a delivery solution or formulation for perilymphatic, subcutaneous, or intramuscular delivery in some embodiments is higher than that typically delivered intravenously. In some embodiments, the concentration of white blood cells in the delivery solution or formulation for perilymphatic, subcutaneous, or intramuscular delivery is greater than about 1.5×10⁸ cells/ml, about 5×10⁸ cells/ml, about 1×10⁹ cells/ml to 1.2×10⁹ cells/ml.

In illustrative embodiments, cells, for example mixtures of modified and unmodified lymphocytes or unmodified cells alone as discussed herein, or, in other embodiments, viral particles (e.g., RIPs) are formulated in a delivery solution or cell formulation such that they are capable of, effective for, and adapted for perilymphatic, intranodal, subcutaneous, or intramuscular administration. In fact, certain embodiments of commercial container and kit aspects provided herein, are or include a container of sterile perilymphatic, intranodal, subcutaneous, and/or intramuscular delivery solution, which in some embodiments is stored refrigerated. Such delivery solutions are capable of, and in illustrative embodiments effective for, and in further illustrative embodiments adapted for, perilymphatic, subcutaneous, intranodal, or intramuscular administration, and in illustrative embodiments subcutaneous administration.

To accomplish this, such delivery solutions and/or formulations, e.g., RIP formulations and cell formulations, typically have a pH and ionic composition that provides an environment in which RIPs to be administered retain their transducing ability (i.e., are effective for, compatible with, or even adapted for transducing cells in vivo, in illustrative embodiments NK cells and/or T cells) and/or cells (e.g., NK cells and/or T cells) to be administered can survive until they are administered, for example for at least 1 hour, and typically can survive for at least 4 hours. Such pH is typically between pH 6.5 to 8.0 or 7.0 and 8.0 or 7.2 to 7.6 and can be maintained by a buffer such as a phosphate buffer or bicarbonate present at a concentration effective for maintaining pH in a target range. In any of the delivery solution and/or formulation aspects and embodiments disclosed herein, a delivery solution and/or a formulation can include one or more of any of the buffers or salts, including concentrations, disclosed in the Exemplary Embodiments section, for example, PBS, HBSS, saline, Ringer's lactate solution, Plasma-Lyte, and others. In any of the delivery solution and/or formulation aspects and embodiments disclosed herein, a delivery solution and/or a formulation can include one or more, for example two or more, three or more, four or more, or five or more, of any other components, including concentrations, disclosed in the Exemplary Embodiments section, for example, DMSO, human serum albumin (HSA), colloids (e.g., dextran (40 kDa to 2 MDa), hetastarch, albumin, PEG (5 kDa-100 kDa)), sugars (e.g., dextrose, lactose, sucrose, or trehalose), and others.

In some embodiments, a delivery solution and/or a formulation is or includes a multiple electrolyte solution. For example, a delivery solution can be or include a sterile, nonpyrogenic isotonic solution in a container, such as a single dose container. Such solution in certain embodiments is suitable or adapted for intravenous administration or intraperitoneal administration as well as perilymphatic, subcutaneous, and/or intramuscular administration. In any of the delivery solution and/or formulation aspects and embodiments disclosed herein, a delivery solution and/or a formulation can include any of the multiple electrolyte solutions, including concentrations, disclosed in the Exemplary Embodiments section, for example, the multiple electrolyte injection solution can be Plasma-Lyte A Injection pH 7.4 available from various commercial suppliers, and others.

In some embodiments, a delivery solution and/or a formulation is frozen before being thawed and administered to a subject. In some embodiments, the delivery solution and/or the formulation can be stored at less than 0, −15, or −70° C. for a certain number of days. In any of the delivery solution and/or the formulation aspects and embodiments disclosed herein, the delivery solution and/or the formulation can include any of the freezing storage temperatures and times disclosed in the Exemplary Embodiments section. In some embodiments, the delivery solution and/or formulation can be frozen for 1, 2, 3, 4, 5, or 6 days, or 1, 2, 3, or 4 weeks, or 1, 2, 3, 4, 5, 6, 9, or 12 months, or indefinitely, before they are administered to, or in illustrative embodiments of cell formulations and delivery solutions comprising cells, re-administered back to the subject. During the time period in which the RIPs or cells are frozen, or any time before administration to the subject, or re-administration back to the subject, the RIPs and/or cells can be tested for various quality control attributes disclosed elsewhere herein, for example, viral concentration, purity, and/or potency, and/or one or more cell and/or gene therapy quality control tests.

In other embodiments, a delivery solution and/or a formulation is never frozen before being administered to a subject. In some embodiments, the delivery solution and/or the formulation can be stored at 2 to 8° C. for a certain number of days. In any of the delivery solution and/or formulation aspects and embodiments disclosed herein, the delivery solution and/or the formulation can include any of the non-freezing storage temperatures and times disclosed in the Exemplary Embodiments section.

In some embodiments, a delivery solution and/or a formulation includes DMSO. In illustrative embodiments, the delivery solution and/or the formulation can include about 6% DMSO (v/v). In some embodiments, the delivery solution and/or the formulation contains no DMSO. In any of the delivery solution and/or formulation aspects and embodiments disclosed herein, the delivery solution and/or the formulation can include any of the DMSO concentrations disclosed in the Exemplary Embodiments section.

In some embodiments, a delivery solution and/or a formulation includes a colloid. In some embodiments, the delivery solution and/or the formulation can include one or more of dextran (40 kDa to 2,000 kDa, or 40 kDa to 2×10⁶ kDa), hetastarch, albumin, PEG (5 kDa-100 kDa). In some embodiments, a delivery solution and/or a formulation includes human serum albumin (HSA). In illustrative embodiments, the delivery solution and/or the formulation can include 2.5% to 7.5% HSA (w/v) (e.g., 25 to 75 mg/ml). In some embodiments, the delivery solution or the formulation includes no HSA. In any of the delivery solution and/or formulation aspects and embodiments disclosed herein, the delivery solution and/or the formulation can include any of the colloids and respective concentrations disclosed in the Exemplary Embodiments section.

In some embodiments, a delivery solution and/or a formulation includes 1% to 10% DMSO and 0.20% to 5% HSA. In further illustrative embodiments, the delivery solution and/or the formulation includes 2% to 8%, 3% to 7%, or 4.5% to 8% DMSO and 0.25% to 7.5%, 0.25% to 6%, or 0.25% to 5% HSA. In other illustrative embodiments, the delivery solution and/or the formulation includes 5% to 7.5% DMSO and 4% to 6% HSA.

In some embodiments, a delivery solution and/or a formulation includes a sugar. In some embodiments, the delivery solution and/or the formulation can include one or more of dextrose, lactose, trehalose, and/or sucrose. In some embodiments, the delivery solution and/or the formulation can include 3 to 7% dextrose (w/v) (e.g., 30 to 70 mg/ml). In some embodiments, the delivery solution and/or the formulation can include 2 to 8% lactose. In some embodiments, the delivery solution and/or the formulation can include 2 to 8% sucrose. In some embodiments, the delivery solution and/or the formulation can include 2 to 8% trehalose. In any of the delivery solution and/or formulation aspects and embodiments disclosed herein, the delivery solution and/or the formulation can include any of the sugars and respective concentrations disclosed in the Exemplary Embodiments section.

Other components that can be included in a delivery solution and/or formulation are disclosed in more detail herein and in the Exemplary Embodiments, and can be delivered either in the same delivery solution and/or formulation or in different delivery solutions and/or formulations, e.g., a delivery solution and a RIP formulation, or a first and second delivery solution. Furthermore, these other components can be delivered along with the delivery solution and/or formulation, or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the first delivery solution and/or formulation. Furthermore, the persistence of genetically modified CAR-T cells near the site of subcutaneous administration further demonstrates an advantage of certain embodiments provided herein wherein the subcutaneous administration is performed near (e.g., within 1, 1, 2, 3, 4, 5, 10, 20, or 30 cm) a site of neoplastic (e.g., cancerous) cells, such as a tumor, or an organ comprising a tumor, including for example, the spleen or lymph nodes in the case of blood cancers.

In some embodiments of any of the delivery solutions and/or formulations provided herein, the delivery solution and/or formulation can be substantially free of bovine protein as disclosed in the Exemplary Embodiments herein. In some embodiments of any of the delivery solutions and/or formulations provided herein, the delivery solution and/or formulation can be substantially free of non-human and non-viral protein as disclosed in the Exemplary Embodiments herein.

The purity of RIPs in a delivery solution or RIP formulation can be determined using the ratio of the amount of protein from the host cells used to generate the RIPs to the transducing units (amount host cell protein/TU). In some embodiments, the ratio of host cell protein to TUs can be 10, 5, 3, 2, or 1 ng or less host cell protein/TU or 750, 500, 400, 300, 200, 100, 50, 40, 30, 20, or 10 μg or less host cell protein/TU. In any of the aspects and embodiments herein that generate RIPs, the host cells used to generate the RIPs can be human cells. In some embodiments, the host cell can be primary cells. In some embodiments, the host cell can be immortalized cells. In some embodiments, the host cells can be HEK, HEK-293, HEK-293T, HEK-293E, HEK-293 FT, HEK-293S, HEK-293SG, HEK-293 FTM, HEK-293SGGD, HEK-293A, 293RTV, GP2-293, MDCK, C127, COS-7, A549, HeLa, CHO, mouse myeloma, PerC6, 91-1, or Vero cells. In illustrative embodiments, the host cells are HEK, HEK293, HEK293T, HEK293A, PerC6 or 91-1.

The potency of RIPs present in a delivery solution or RIP formulation can be determined using the ratio of the TUs to the ng of p24 protein. In some embodiments, the ratio of the TUs to the ng of p24 protein can be 100, 200, 300, 400, 500, 1,000, 4,000, 10,000, 12,500, or 15,000 or more TUs/ng of p24 protein.

In some embodiments, the concentration of RIPs present in a delivery solution and/or RIP formulation can be at least 1×10⁶, 5×10⁶, 1×10⁷, 5×10⁷, 1×10⁸, 2×10⁸, 5×10⁸, or 1×10⁹ TU/ml. In some embodiments, the concentration of RIPs present in a delivery solution and/or RIP formulation can be any of the concentrations disclosed in the Exemplary Embodiments section herein.

In some embodiments such as those embodiments in which the samples do not undergo a PBMC isolation or granulocyte depletion procedure, at least 5%, at least 10%, at least 20%, at least 25%, at least 30%, at least 40%, at least 50%, or at least 75% of the neutrophils, basophils, and/or eosinophils present in a blood sample that is subjected to a method for modifying herein or co-administered with a RIP formulation as unmodified cells (i.e., wherein the blood has not previously been contacted with RIPs), are present in the cell formulation, including at the time of the optional delivery (i.e., administering) step. In some embodiments such as those embodiments in which the samples do not undergo a B cell depletion procedure, at least 5%, at least 10%, at least 20%, at least 25%, at least 30%, at least 40%, at least 50%, or at least 75% of the B cells present in a blood sample that is subjected to a method for modifying herein or co-administered with a RIP formulation as unmodified cells, are present in the cell formulation, including at the time of the optional delivery step. In some embodiments such as those embodiments in which the samples do not undergo a monocyte depletion procedure, at least 5%, at least 10%, at least 20%, at least 25%, at least 30%, at least 40%, at least 50%, or at least 75% of the monocytes present in a blood sample that is subjected to a method for modifying herein or co-administered with a RIP formulation as unmodified cells, are present in the cell formulation, including at the time of the optional delivery step.

In some embodiments, and in illustrative embodiments in which the cell formulation is administered subcutaneously or intramuscularly, the volume of the cell formulation including the modified and/or unmodified lymphocytes is less than traditional CAR-T methods, which typically are infusion-delivery methods, and can be less than, or less than about 1 ml, about 2 ml, about 3 ml, about 4 ml, about 5 ml, about 10 ml, about 15 ml, about 20 ml, or about 25 ml.

The advantageously short time between drawing (collecting) blood and reintroducing the unmodified or modified lymphocytes into the subject means that in some embodiments, some lymphocytes are associated with the recombinant nucleic acid vectors, and in illustrative embodiments the replication incompetent recombinant retroviral particles, are not yet genetically modified. In some embodiments, at least 5% of the modified lymphocytes are not genetically modified. In some embodiments, the modified lymphocytes are genetically modified and contain the polynucleotide, either extrachromosomal or integrated into the genome. In some embodiments, the polynucleotide can be extrachromosomal in at least 5% of the modified lymphocytes. In some embodiments, at least 5% of the modified lymphocytes are not transduced. In some embodiments including co-administration with a RIP formulation or RIPs in a delivery solution, the lymphocytes have not been contacted with a RIP disclosed herein, and thus none of the lymphocytes are modified, genetically modified, or transduced with such a RIP.

The short contacting time in certain embodiments also results in many of the modified lymphocytes in cell formulations herein, having on their surfaces, binding polypeptides, fusogenic polypeptides, and in some embodiments T cell activation elements that originated on the surface of retroviral particles, either through association with the recombinant retroviral particles or by fusion of the retroviral envelopes with the plasma membranes, including at the time of the optional delivery step. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation include a pseudotyping element and/or a T cell activation element, e.g., a T cell activating antibody. In some embodiments, the pseudotyping element and/or T cell activation element can be bound to the surface of the modified lymphocytes through, for example, a T cell receptor, CD28, OX40, 4-1BB, ICOS, CD9, CD53, CD63, CD81, CD82, and/or the pseudotyping element and/or T cell activation element can be present in the plasma membrane of the modified lymphocytes. In some embodiments including co-administration with a RIP formulation or RIPs in a delivery solution, the lymphocytes have not been contacted with a RIP disclosed herein, and thus none of the lymphocytes include a pseudotyping element or a T cell activation element bound to the surface of the lymphocytes and thus none of the lymphocytes are modified, genetically modified, or transduced lymphocytes. In some aspects, cell formulations are provided herein that include for example T cells and/or NK cells. Such formulations, in illustrative embodiments are provided by methods provided herein. In one aspect, provided herein is a cell formulation comprising a population of unmodified, modified, genetically modified, transcribed, transfected, and/or stably integrated T cells and/or NK cells, including in non-limiting examples, CAR-T cells and/or NK cells in a delivery solution.

Due to the advantageously short time lymphocytes are contacted with recombinant nucleic acid vectors and modified lymphocytes are ex vivo after such contacting in some illustrative embodiments provided herein, in these embodiments some or all of the T and NK cells do not yet express the recombinant nucleic acid or have not yet integrated the recombinant nucleic acid into the genome of the cell, and some of the retroviral particles in embodiments including these, may be associated with, but may have not fused with the target cell membrane, before being used or included in any of the methods or compositions provided herein, including, but not limited to, being introduced or reintroduced back into a subject, or before being used to prepare a cell formulation. Thus, various cell formulation aspects and embodiments are provided herein that can be produced, for example, from these illustrative methods provided herein, such as for example, rapid point of care methods that in illustrative embodiments involve subcutaneous administration. Such cell formulations, including but not limited to those set out immediately below and in the Exemplary Embodiments section herein, can exist at the time of collection of cells after they are contacted with a recombinant retroviral vector and optionally rinsed, and can exist up to and including at the time of administration to a subject, in illustrative embodiments subcutaneously.

In some embodiments, provided herein are cell formulations comprising T cells and/or NK cells, wherein less than 90%, 80%, 75%, 70%, 60%, 50%, 40%, 30%, 25%, 20%, 10%, or 5% of the cells in the cell formulation are T cells and/or NK cells. In some embodiments, none of the cells have been contacted with a RIP disclosed herein and thus none of the cells are modified, genetically modified, or transduced. In some embodiments, cell formulations comprising lymphocytes, NK cells, and/or T cells, are provided wherein at least 4%, 5%, 10%, 15%, 20%, 25%, 30%, 35%, 40%, 45%, 50%, 55%, 60%, 65%, 70%, 75%, 80%, 85%, 90%, or 95% of the lymphocytes, NK cells, and/or in illustrative embodiments T cells in the cell formulation are modified cells, for example, modified with polynucleotides comprising nucleic acids that encode anti-idiotype polypeptides provided herein. Such polynucleotides can optionally encode a CAR, TCR, inhibitory RNA, or LE, as provided herein. In some embodiments, between 4%, 5%, 10%, 15%, 20%, 25%, 30%, 35%, 40%, 45%, 50%, 55%, 60%, 65%, and 70% of the lymphocytes are modified on the low end of the range and 10%, 15%, 20%, 25%, 30%, 35%, 40%, 45%, 50%, 55%, 60%, 65%, 70%, 75%, 80%, 85%, 90%, and 95% of the lymphocytes are modified cells on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified lymphocytes within the cell formulation are not genetically modified, transduced, or stably transfected. In some embodiments, between 5%, 10%, 15%, 20%, 25%, 30%, 35%, 40%, 45%, 50%, 55%, 60%, 65%, and 70% of the modified lymphocytes are not genetically modified, transduced, or stably transfected on the low end of the range and 10%, 15%, 20%, 25%, 30%, 35% 40%, 45%, 50%, 55%, 60%, 65%, 70%, 75%, 80%, 85%, 90%, 95%, 96%, 97%, 98%, and 99% or all of the modified lymphocytes are not genetically modified, transduced, or stably transfected on the high end of the range, for example between 5% and 95%, 10% and 90%, 25% and 75%, and 25% and 95%. In some embodiments, the polynucleotide of genetically modified lymphocytes can be either extrachromosomal or integrated into the genome in these cell formulations that are formed after contacting and incubation, and at the time of optional administration. In some embodiments of these cell formulations, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the genetically modified lymphocytes have an extrachromosomal polynucleotide. In some embodiments, between 5%, 10%, 15%, 20%, 25%, 30%, 35%, 40%, 45%, 50%, 55%, 60%, 65%, and 70% of the modified or genetically modified lymphocytes have an extrachromosomal polynucleotide on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes have an extrachromosomal polynucleotide on the high end of the range, for example between 5% and 95%, 10% and 90%, 25% and 75%, and 25% and 95%. In some embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or all of the modified or genetically modified lymphocytes are not transduced or stably transfected in these cell formulations, for example as a result of methods for genetically modifying T cells and/or NK cells provided herein. In some embodiments, between 5%, 10%, 15%, 20%, 25%, 30%, 35%, 40%, 45%, 50%, 55%, 60%, 65%, and 70% of the modified or genetically modified lymphocytes are not transduced on the low end of the range and 10%, 15%, 20%, 25%, 30%, 35%, 40%, 45%, 50%, 55%, 60%, 65%, 70%, 75%, 80%, 85%, 90%, 95%, 96%, 97%, 98%, and 99% or all of the modified or genetically modified lymphocytes are not transduced or stably transfected on the high end of the range, for example between 5% and 95%, 10% and 90%, 25% and 75%, and 25% and 95%.

In certain embodiments disclosed herein including subcutaneous delivery of a solution, and cell formulations that are adapted for subcutaneous delivery, fewer of the modified or genetically modified lymphocytes can engraft if delivered intravenously compared to when delivered subcutaneously. In some embodiments, at least 5%, 10%, 15%, 20%, 25%, 30%, 35%, 40%, 45%, 50%, 55%, 60%, 65%, 70%, 75%, 80%, 85%, 90%, or 95% fewer lymphocytes engraft when delivered intravenously compared to when delivered subcutaneously.

In some embodiments, cell formulations, including such formulations in existence at the time of collection of cells after they are contacted with a recombinant retroviral vector and optionally rinsed, and existing up to and including the time of administration to a subject, comprise at least two of unmodified lymphocytes, modified lymphocytes, and genetically modified lymphocytes. In some embodiments, such cell formulations comprise more unmodified lymphocytes than modified lymphocytes. In some embodiments of such cell formulations that are produced by methods provided herein, the percent of T cells and NK cells that are modified, genetically modified, transduced, and/or stably transfected is at least 5%, at least 10%, at least 15%, or at least 20%. As illustrated in the Examples herein, in exemplary methods provided herein for transducing lymphocytes in whole blood, between 1% and 20%, or between 5% and 20%, or between 1% and 15%, or between 5% and 15%, or between 7% and 12% or about 10% of lymphocytes, and in some embodiments of T cells and/or NK cells in the whole blood that is added to a reaction mixture or that is used to create a reaction mixture, are genetically modified and/or transduced and present in resultant cell formulations. In some embodiments, the lymphocytes are not contacted with a recombinant nucleic acid vector, such as a replication incompetent recombinant retroviral particle, and are not modified. In certain illustrative embodiments, the lymphocytes are tumor infiltrating lymphocytes. In some embodiments, the lymphocytes are tumor infiltrating lymphocytes before or after the tumor infiltrating lymphocytes are in contact a recombinant nucleic acid vector. In some embodiments, the lymphocytes comprise both tumor infiltrating lymphocytes and T cells and/or NK cells before or after the T cells and/or NK cells contact a recombinant nucleic acid vector.

In some embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in the cell formulation do not express a CAR, or a transposase in certain embodiments, and/or do not have a CAR associated with their cell membrane. In other embodiments, provided herein are cell formulations wherein at least 25%, 30%, 40%, 45%, 50%, 55%, 60%, 65%, 70%, 75%, 80%, 85%, 90%, 95%, 96%, 97%, 98%, 99%, or all of the modified T and/or NK cells in a cell formulation contain recombinant viral reverse transcriptase or integrase. Not to be limited by theory, unlike traditional CAR-T cell processing methods where cells are cultured ex-vivo for days or weeks and many cell divisions, in illustrative methods provided herein, where T cells and/or NK cells are contacted with retroviral particles to modify the T cells and/or NK cells within hours of delivery, some or most of the reverse transcriptase and integrase present within the retroviral particles that moves into a T cell and/or NK cell after it fuses with a retroviral particle, would still be present in the modified T cells and/or NK cells at the time of delivery. In some embodiments, provided herein are cell formulations wherein at least 25%, 30%, 35%, 40%, 45%, 50%, 55%, 60%, 65%, 70%, 75%, 80%, 85%, 90%, 95%, 96%, 97%, 98%, 99%, or all of the modified T and NK cells in a cell formulation do not express the recombinant mRNA (e.g., encoding a CAR and/or a recombinant transposase). In some embodiments, provided herein are cell formulations wherein at least 20%, 25%, 30%, 35%, 40%, 45%, 50%, 55%, 60%, 65%, 70%, 75%, 80%, 85%, 90%, 95%, 96%, 97%, 98%, 99%, or all of the modified T and NK cells in such cell formulation do not have the recombinant nucleic acid stably integrated into their genomes. In some embodiments, greater than 50%, 60%, 70%, 75%, 80% or 90% of the cells, NK cells, and/or T cells in a cell formulation are viable.

In further embodiments, cell formulations comprising modified lymphocytes that can be introduced or reintroduced in methods herein, include monocytes and/or B cells. In some embodiments, some of the B cells are modified during a contacting step when they are contacted by recombinant nucleic acid vectors, for example, naked DNA vectors, or in illustrative embodiments replication incompetent recombinant retroviral particles. In some embodiments, at least some but not more than 5%, 10%, 15%, 20%, 25%, 30%, 35%, 40%, 45%, 50%, 55%, 60%, 65%, 70%, 75%, 80%, 85%, 90%, or 95% of the B cells are modified in cell formulations, which can optionally be administered or re-administered. In illustrative embodiments, some of the B cells are not modified in such formulations and methods. In further illustrative embodiments, at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the B cells are not modified in such formulations and methods. Thus, in some embodiments, modified lymphocytes are present in cell formulations along with unmodified lymphocytes, which optionally are delivered to a subject intramuscularly or subcutaneously. In some embodiments, the modified lymphocytes in the cell formulations and optionally introduced into the subject can be allogeneic lymphocytes. In such embodiments, the lymphocytes are from a different person, and the lymphocytes from the subject are not modified. In some embodiments, no blood is collected from the subject to harvest lymphocytes.

Neutrophils, in illustrative embodiments, are present in the cell formulation, as a nonlimiting example a cell formulation for delivering modified T cells and/or NK cells subcutaneously, at a concentration too high for intravenous delivery when considering the safety of a subject into which the cell formulation is administered. Not to be limited by theory, and as discussed herein elsewhere, the injection or delivery of neutrophils intravenously can lead to pulmonary compromise, for example, as a result of transfusion-related acute lung injury (TRALI) and/or acute respiratory distress syndrome (ARDS). For example, this situation can arise when the method for producing the modified lymphocytes does not involve a PBMC enrichment step before the cell formulation comprising the modified lymphocytes is prepared, and before the solution is optionally delivered subcutaneously to a subject. Thus, in some embodiments, neutrophils are present in the cell formulation, for example at the time of the optional delivery step. More specifically, in some embodiments, at least 5%, at least 10%, at least 20%, at least 25%, at least 30%, at least 40%, at least 50%, or at least 75% of the neutrophils present in a blood sample that is subjected to a method for modifying herein, are present in the cell formulation, including at the time of the optional delivery step. In some embodiments, at least 5%, at least 10%, at least 20%, at least 25%, at least 30%, at least 40%, at least 50%, at least 60%, at least 70%, or at least 75% of the cells present in the cell formulation are neutrophils, including at the time of the optional delivery step. In some embodiments, between 5%, 10%, 15%, 20%, 25%, 30%, or 40% of the cells present in the cell formulation are neutrophils at the low end of the range and 30%, 40%, 50%, 60%, 70%, or 75% of the cells present in the cell formulation are neutrophils at the high end of the range, including at the time of the optional delivery step, for example between 5% and 50%, 20% and 50%, 30% and 75%, or 50% and 75% of the cells present in the cell formulation are neutrophils, including at the time of the optional delivery step.

In some embodiments, at least 5%, at least 10%, at least 20%, at least 25%, at least 30%, at least 40%, at least 50%, or at least 75% of the monocytes present in a blood sample that is subjected to a method for modifying herein, are present in a cell formulation, including at the time of the optional delivery step. In some embodiments, at least 5%, at least 10%, at least 20%, at least 25%, at least 30%, at least 40%, at least 50%, or at least 75% of the B cells present in a blood sample that is subjected to a method for modifying herein, are present in the resulting cell formulation, including at the time of the optional delivery step. In some embodiments, the cell formulation can include a PBMC fraction, which includes the modified T and NK cells. In some embodiments, at least 1%, 2%, 3%, 4%, 5%, 10%, 15%, 20%, 25%, 50%, 75%, 80%, 85%, 90%, or 95%, or between 1% and 95%, 5% and 95%, 5% and 50%, or 10% and 50% of the modified T and NK cells in a cell formulation are genetically modified.

The volume of delivery solution, RIP formulation, or cell formulation or other solution administered varies depending on the route of administration, as provided elsewhere herein. Delivery solutions, RIP formulations, or cell formulations injected perilymphatically, subcutaneously, or intramuscularly typically have smaller volumes than those delivered via infusion. In some embodiments, the volume of the delivery solution, RIP formulation, or cell formulation is not more than 1 ml, 2 ml, 3 ml, 4 ml, 5 ml, 10 ml, 15 ml, 20 ml, 25 ml, 30 ml, 35 ml, 40 ml, 45 ml, or 50 ml. In some embodiments, the volume of the delivery solution, RIP formulation, or cell formulation can be between 0.20 ml, 0.25 ml, 0.5 ml, 1 ml, 2 ml, 3 ml, 4 ml, 5 ml, 10 ml, 15 ml, 20 ml, or 25 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, 50 ml, 75 ml, 100 ml, 125 ml, 250 ml, 500 ml, or 1000 ml on the high end of the range. Thus, as non-limiting examples, the volume can be between 0.2 ml and 10 ml, 0.5 ml and 10 ml, 0.5 and 2 ml, 1 ml and 250 ml, 1 ml and 100 ml, 10 ml and 100 ml, or 1 ml and 10 ml. In certain illustrative embodiments, a delivery solution, RIP formulation, or cell formulation can be less than 10 ml, between 1 ml and 25 ml, and in illustrative embodiments between 1 ml and 3 ml, between 1 ml and 5 ml, or between 1 ml and 10 ml. In illustrative embodiments, the volume of the delivery solution, RIP formulation, or cell formulation can be between 0.20 ml, 0.25 ml, 0.5 ml, 1 ml, 2 ml, 3 ml, 4 ml, and 5 ml on the low end of the range and 0.5 ml, 1 ml, 2 ml, 3 ml, 4 ml, 5 ml, 10 ml, 15 ml, 20 ml, 25 ml, 30 ml, 35 ml, 40 ml, 45 ml, and 50 ml on the high end of the range. In an exemplary embodiment of a cell formulation, a 70 kg subject is dosed at 1.0×10⁶ T cells/kg by administering 1 ml of a delivery formulation of T cells at 7.0×10⁷ cells/ml subcutaneously. In some embodiments, a delivery solution, RIP formulation, or cell formulation can include hyaluronidase when the volume of the solution is at least 2 ml, 3 ml, 4 ml, 5 ml, 10 ml, 15 ml, 20 ml, or 25 ml. In embodiments herein wherein lymphocytes are filtered especially after they are modified, and/or especially where transduction is performed on top of a filter, the delivery solution can be used to resuspend and/or elute cells from the filter in volumes that can be those provided above. As such, in some embodiments, a delivery solution provided herein is an elution solution.

In some embodiments, unmodified, modified and in illustrative embodiments genetically modified lymphocytes are introduced or reintroduced into the subject by intradermal, intratumoral or intramuscular administration and in illustrative embodiments, perilymphatic or subcutaneous administration using a cell formulation present in a subcutaneous delivery device, such as a sterile syringe that is adapted to deliver a solution subcutaneously. In some embodiments, a subcutaneous delivery device is used that holds a solution (e.g., a delivery solution, RIP formulation, or cell formulation herein) and has an open or openable end, which in illustrative embodiments is the open end of a needle, for administering the solution (e.g., delivery solution, RIP formulation, or cell formulation) subcutaneously from the liquid holding portion of the device. Such a subcutaneous delivery device is effective for, and in illustrative embodiments adapted for subcutaneous delivery, or effective to inject subcutaneously or adapted to inject subcutaneously. Non-limiting examples of subcutaneous delivery devices that are adapted to deliver a solution subcutaneously include subcutaneous catheters, such as indwelling subcutaneous catheters, such as for example, the Insuflon® (Becton Dickinson) and needless closed indwelling subcutaneous catheter systems, for example with wings, such as for example, the Saf-T-Intima® (Becton Dickinson). In some embodiments, the delivery device can include a pump, for example an infusion pump or a peristaltic pump. In some embodiments, the delivery solution, RIP formulation, or cell formulation is fluidly connected to any of the needles disclosed herein, for example a needle compatible with, effective for, adapted for, or adapted to deliver subcutaneously or effective to deliver subcutaneously. In illustrative embodiments, the needle can have a gauge between 26 and 30. In some embodiments, the subcutaneous delivery device is a subcutaneous delivery pen. Such a pen can include a syringe effective to deliver subcutaneously or adapted to deliver subcutaneously enclosed within a housing and can include a needle guard. Examples of such pens include pens used to deliver sumatriptan. In some embodiments, said delivery solution, RIP formulation, or cell formulation is present in a subcutaneous delivery device, for example a syringe, with a needle that has penetrated the skin of a subject where RIPs and/or unmodified and modified T cells and/or NK cells are present in the syringe (i.e., the subject receiving the subcutaneous injection is the source of the RIPs or autologous cells being injected), and in some embodiments is located with its open end in the subcutaneous tissue of the subject. In illustrative embodiments, the subcutaneous delivery device (e.g., syringe) can include a needle that is suitable for subcutaneous administration. Subcutaneous administration typically uses needles with smaller diameters than used with intravenous catheters for blood infusion, which for example can employ a 16 gauge needle. A delivery device such as a syringe that is compatible with intramuscular and, in illustrative embodiments, subcutaneous delivery, is any delivery device (e.g., syringe) that can be successfully used for intramuscular or subcutaneous delivery, and includes those delivery devices (e.g., syringes) that are effective for and adapted for intramuscular or subcutaneous delivery, plus general purpose syringes and syringes that are specifically designed for other purposes and that can be successfully employed for intramuscular or subcutaneous delivery in at least some embodiments. As is known, for subcutaneous injection, in illustrative embodiments using a syringe, a needle is inserted through the skin at a 450 to 900 angle. Thus, some embodiments include injecting a delivery solution, RIP formulation, or cell formulation subcutaneously at an angle of 45° to 90° with respect to the skin, as well as a delivery solution, RIP formulation, or cell formulation contained within a syringe or other subcutaneous delivery device, having a needle at a 45° to 90° angle to the skin of a subject. A syringe that is effective for intramuscular and, in illustrative embodiments, subcutaneous delivery, or effective to inject intramuscularly or subcutaneously, is a syringe with parameters that are typically effective for intramuscular or subcutaneous delivery, for example, a needle with a gauge between 20 and 22 and a length between 1 inch and 1.5 inches is typically effective for intramuscular delivery and a needle with a gauge between 26 and 30 and a length between 0.5 inches and 0.625 inches is typically effective for subcutaneous delivery. A syringe that is adapted for subcutaneous delivery, or adapted to inject subcutaneously, is any syringe that is specifically made for subcutaneous delivery. One such syringe adapted for subcutaneous delivery uses a core annular flow that allows subcutaneous delivery of highly concentrated biological drug formulations not normally deliverable subcutaneously (Jayaprakash V et al. Adv Healthc Mater. 2020 Aug. 24; e2001022). Another syringe adapted for subcutaneous delivery uses a shorter needle than generally used (Pager A, Expert Opin Drug Deliv. 2020 Aug. 9; 1-14). Another syringe adapted for subcutaneous delivery uses a 29G/5-bevel needle with a Thermo Plastic Elastomer (TPE) needle shield (Jaber A et al. BMC Neurol. 2008 Oct. 10; 8:38). In illustrative embodiments, the outer diameter of the needle is less than 0.026″. In some embodiments, the outer diameter of the needle is at most 0.01625″, 0.01865″, 0.01825″, 0.02025″, 0.02255″, or 0.02525″. In some embodiments, the needle is a 17, 18, 19, 20, 21, 22, 23, 24, 25, 26, 26s, 27, 28, 29, or 30 gauge needle. In some embodiments, the length of the needle is not more than 1 inch or 0.5 inches. In illustrative embodiments, the needle is 26, 26s, 27, 28, 29, or 30 gauge needle and the length of the needle is between 0.5 inches and 0.625 inches. In some embodiments, the needle can be a winged infusion set, also known as a butterfly or scalp vein needle. In some embodiments, the introduction or reintroduction can be performed using a subcutaneous catheter.

Not to be limited by theory, in contrast to intravenous delivery in which the components of the delivery solution, RIP formulation, or cell formulation rapidly disperse, subcutaneous and intramuscular delivery methods provided herein permit the components of the delivery solution, RIP formulation, or cell formulation to remain in close proximity within a subject, for example in illustrative embodiments for up to several days, several weeks, or even several months as a controlled release while creating a local environment for T cell and/or NK cell activation and expansion while maintaining properties similar to what T and NK cells encounter in the lymphoid organs such as the spleen or lymph node. While the absorption of large protein molecules over 20 kDa such as antibodies from subcutaneous sites are absorbed into the blood through the lymphatics over 24 to 72 hours, controlled release of modified, genetically modified, and/or transduced T or NK cells from a cell formulation injected at a local injection site using perilymphatic, subcutaneous, or intramuscular methods provided herein, were found to involve an initial expansion phase at the site of injection before at least some and typically most of the modified cells migrate through blood vessels and lymphatics to the site of target expression, such as a tumor, and then be detectable throughout the body. RIPs from a RIP formulation injected at a local injection site using perilymphatic, subcutaneous, or intramuscular methods provided herein can transduce T and NK cells present in the subject, or when co-administered with PBMCs, for example, T and/or NK cells, isolated from the subject, can transduce the co-administered T and/or NK cells. In some embodiments the local injection controlled release of modified, genetically modified, and/or transduced cells (either from the cell formulation or later modified from a RIP formulation) will result in genetically modified cells expanding at the site of subcutaneous administration for days (e.g., for up to 5, 7, 14, 17, 21, or 28 days) or months (e.g., for up to 1, 2, 3, 6, 12, or 24 months) with genetically modified CAR-T cells or CAR-NK cells migrating away from the site of subcutaneous administration to other sites of the body, for example to tumors. Thus, genetically modified CAR-T cells can appear in lymphatics or circulation migrating away from a subcutaneous administration site after days (e.g., 1, 2, 3, 4, 5, 6, or 7 days), weeks (e.g., 1, 2, 4, or 4 weeks), and even months (e.g., 1, 2, 3, 6, 12, or 24 months) after modified T cells and/or NK cells are injected subcutaneously into a subject.

This persistence of genetically modified T cells and/or NK cells, such as CAR-T cells, subcutaneously provides an advantageous local environment where other components native or non-native to the subject, such as molecules (ions), macromolecules (e.g., DNA, RNA, peptides, and polypeptides) and/or other cells that can affect the modified CAR-T cells, can be recruited or delivered subcutaneously at or near the site of delivery of the modified CAR-T cells. In fact, tertiary lymphoid structures comprising lymphatic vasculature have been observed after delivery of modified T cells and/or NK cells. Not to be limited by theory, it is believed that such lymphatic vasculature provides a venue for modified T cells and/or NK cells administered subcutaneously to access the local lymphatic circulation, after which they can gain access to the systemic circulation and, for example, access the blood. Such tertiary lymphoid structures have been observed to comprise activated lymphoid cells. Tertiary lymphoid structures can comprise one or more of CD4+ lymphocytes, CD8+ lymphocytes, CD68+ antigen presenting cells (monocytes, macrophages and dendritic cells), CD56+ lymphocytes, CD57+ lymphocytes, and NKG2D+ lymphocytes. Accordingly, provided herein are lymphoid structures comprising aggregates of actively dividing genetically modified T cells and/or NK cells and lymphatic vasculature in proximity to such aggregates. In some embodiments, tertiary lymphoid structures and/or the genetically modified CAR-T cells can persist near a site of subcutaneous administration for at least 1, 2, 3, 4, 5, 6, or 7 days, 1, 2, 3, 4, 5, 6, 7, or 8 weeks, or 1, 2, 3, 4, 5, 6, 7, 8, 9, 10, 11, 12, or 24 months. In illustrative embodiments, tertiary lymphoid structures and/or the genetically modified CAR-T cells persist near the site of subcutaneous administration for at least 1, 2, 3, 4, 5, 6, or 7 days, 1, 2, 3, or 4 weeks, or 1, 2, or 3 months. In some embodiments, tertiary lymphoid structures and/or the genetically modified CAR-T cells can persist near a site of subcutaneous administration for between 1 day and 24 months, 7 days and 12 months, 2 weeks and 6 months, 3 weeks and 8 weeks, or 4 weeks and 6 weeks. In illustrative embodiments, in some embodiments, tertiary lymphoid structures and/or the genetically modified CAR-T cells can persist near a site of subcutaneous administration for between 1 week and 3, 4, 5, 6, 7, 8, 9, or 10 weeks, for example between 1 week and 8 weeks, 1 week and 7 weeks, or 1 week and 6 weeks. In some embodiments, at least 10%, 20%, 30%, 40%, 50%, 60%, 70%, 80%, 90%, or 95% of the cells in tertiary lymphoid structures and/or of the genetically modified cells can remain localized within 1, 2, 3, 4, or 5 cm of site of administration. In some embodiments of any of the aspects or embodiments that include administering a cell population, formulation, delivery solution, or RIP, the administering can be performed at least at 2 or more locations in a subject. In some embodiments, the administering can be performed at 2, 3, 4, 5, or more locations in a subject. Accordingly, in some embodiments that include administering of a cell population, formulation, delivery solution, or RIP at least at 2 or more locations in a subject, the subject can have 2 or more tertiary lymphoid structures as disclosed herein. In some embodiments, there can be more than 1 tertiary lymphoid structure, for example, 2, 3, 4, 5, or more. Without being bound by theory, presence of more than 1 tertiary lymphoid structure can help in the engraftment process.

In some embodiments, the expression in lymphocytes of lymphoproliferative elements disclosed herein can increases the percentage of CD8+ cells relative to before expression of the lymphoproliferative element. In some embodiments, at least 60%, 65%, 70%, 75%, 80%, 85%, 90%, or 95% of CD3+ lymphocytes in a cell population expressing a lymphoproliferative element disclosed herein are CD8+ after expressing the lymphoproliferative element for at least 5 days. In some embodiments, at least 60% of CD3+ lymphocytes in a cell population expressing a lymphoproliferative element disclosed herein are CD8+ after expressing the lymphoproliferative element for at least 2, 3, 4, 5, 6, 7, 8, 9, 10, 11, 12, 13, or 14 days. In some embodiments, 40%, 35%, 30%, 25%, 20%, 15%, 10%, 5%, or less of CD3+ lymphocytes in a cell population expressing a lymphoproliferative element disclosed herein are CD4+ after expressing the lymphoproliferative element for at least 5 days. In some embodiments, 40% or less of CD3+ lymphocytes in a cell population expressing a lymphoproliferative element disclosed herein are CD4+ after expressing the lymphoproliferative element for at least 2, 3, 4, 5, 6, 7, 8, 9, 10, 11, 12, 13, or 14 days. In some embodiments, the CD8+ cells are at least 2, 3, 4, 5, 6, 7, 8, 9, 10, 11, 12, 13, 14, 15, 16, 17, 18, 19, or 20 folds more in number than the CD4+ cells. Such cell populations may be in vitro or in vivo. In vivo locations include subcutaneous, in a TLS, intramuscular, intranodal, intraperitoneal, and intratumoral.

Other components that can be delivered along with modified lymphocytes are disclosed in more detail herein, and can be delivered either in the same formulation or in different formulation(s) than the modified T cells and/or NK cells. Furthermore, these other components can be delivered along with the modified T cells and/or NK cells, or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the modified T cells and/or NK cells.

In some embodiments, the cell formulation is compatible with or even adapted for subcutaneous, intratumoral, or intramuscular delivery to keep the cells aggregated locally to enable a controlled release of cells into circulation. The concentration of cells in a cell formulation for subcutaneous, intratumoral, intranodal, or intramuscular delivery in some embodiments is higher than that typically delivered intravenously. In some embodiments the concentration of white blood cells in the cell formulation for subcutaneous or intramuscular delivery is greater than, or greater than about 1.5×10⁸ cells/ml, about 5×10⁸ cells/ml, about 1×10⁹ cells/ml to 1.2×10⁹ cells/ml.

In illustrative embodiments, cells, for example mixtures of modified and unmodified lymphocytes discussed herein, are formulated in a delivery solution such that they are capable of, effective for, and adapted for subcutaneous, intratumoral, intranodal, or intramuscular administration. In fact, certain embodiments of commercial container and kit aspects provided herein, are or include a container of sterile subcutaneous, intratumoral, intranodal, and/or intramuscular delivery solution, which in some embodiments is stored refrigerated. Such delivery solutions are capable of, and in illustrative embodiments effective for, and in further illustrative embodiments adapted for, subcutaneous, intratumoral, intranodal, or intramuscular administration, and in illustrative embodiments subcutaneous administration. To accomplish this, such delivery solutions and resulting cell formulations typically have a pH and ionic composition that provides an environment in which cells to be administered can survive until they are administered, for example for at least 1 hour, and typically can survive for at least 4 hours. In some embodiments, for example, when the replication incompetent recombinant retroviral particle has a polynucleotide that encodes an MRB-CAR, the pH can be between pH 6.0 to pH 7.0, for example, pH 6.2 to pH 7.0, or pH 6.4 to pH 7.0, or pH 6.4 to pH 6.8. The ionic composition of such formulations can for example, include a saline composition with salts, for example 0.8 to 1.0 or about 0.9 or 0.9 percent salts such as sodium chloride. In some embodiments, the delivery solution is or includes PBS/In some embodiments of a delivery solution and resulting cell formulation herein, the concentration of Na⁺ is between 110 mM and 204 mM, the concentration of Cl⁻ is between 98 mM and 122 mM, and/or the concentration of K⁺ is between 3 mM and 6 mM. In illustrative embodiments, a delivery solution and cell formulation comprising the same, contains calcium and/or magnesium. The concentration of calcium can, for example, be between 0.5 mM and 2 mM. The concentration of magnesium can, for example, be between 0.5 mM and 2 mM. In some embodiments, the delivery solution is calcium and magnesium free. In some embodiments, the delivery solution can be Ringer's lactate solution, also known as sodium lactate solution and Hartmann's solution. In illustrative embodiments, Ringer's lactate solution can contain about 130-131 mM sodium, 109-111 mM chloride, 28-29 mM lactate, 4-5 mM potassium, and 1-1.5 mM calcium, and is typically made by mixing sodium chloride (NaCl), sodium lactate (CH₃CH(OH)CO₂Na), calcium chloride (CaCl₂), and potassium chloride (KCl). In some embodiments, the delivery solution can be Plasma-Lyte. In illustrative embodiments, Plasma-Lyte can contain about 150 mM sodium, 5 mM potassium, 1.5 mM magnesium, 98 mM chloride, 27 mM acetate, and 23 mM gluconate. In some embodiments, the delivery solution can include dextrose. In some embodiments, the concentration of dextrose can be at least or about 0.5%, 1%, 2%, 3%, 4%, 5%, 6%, 7%, 8%, 9%, or 10%. In illustrative embodiments, the delivery solution be 5% dextrose in 0.9% NaCl.

In some embodiments, the delivery solution and cell formulations contain human serum albumin and/or heparin. In some embodiments the delivery solution and cell formulation contain up to 5% HSA. In some embodiments, the delivery solution contains at least or about 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml. In some embodiments, the delivery solution contains between 10 mg/ml and 30 mg/ml, 10 mg/ml and 100 mg/ml, 20 and 80 mg/ml, or 40 mg/ml and 60 mg/ml. In some embodiments the delivery solution is PBS comprising 2% HSA. In some embodiments, the delivery solution is DPBS comprising 2% HSA (W/V, i.e., 2 g per 100 ml). In some embodiments, the delivery solution comprises 30-100 U/ml, 40-100 U/ml, 30-60 U/ml, or 60-80 U/ml heparin. In some embodiments, the delivery solution is a saline solution comprising 30-100 U/ml, 40-100 U/ml, 30-60 U/ml, or 60-80 U/ml heparin, with or without 0.5-5%, 1-5% Ir 1-2.5% HSA. Discussion herein regarding concentrations of heparin in reaction mixture aspects, apply equally to delivery solution and cellular formulation aspects. In some embodiments, the delivery solution comprises a saline solution at about pH 7.4 further comprising HSA and sodium bicarbonate.

In some embodiments, the delivery solution is or includes a multiple electrolyte solution suitable for injection into a subject. Such solution in certain embodiments is suitable or adapted for intravenous administration as well as subcutaneous and/or intramuscular administration. In some embodiments, a delivery solution can include a multiple analyte solution for injection into a subject where ach 100 mL contains 526 mg of Sodium Chloride, USP (NaCl); 502 mg of Sodium Gluconate (C₆H₁₁NaO₇); 368 mg of Sodium Acetate Trihydrate, USP (C₂H₃NaO₂·3H₂O); 37 mg of Potassium Chloride, USP (KCl); and 30 mg of Magnesium Chloride, USP (MgCl₂·6H₂O) with a pH adjusted to 7.4 (6.5 to 8.0). In illustrative embodiments, the delivery solution contains no antimicrobial agents. The pH is adjusted with sodium hydroxide. As one example, the multiple electrolyte injection solution can be PLASMA-LYTE A Injection pH 7.4 available from various commercial suppliers.

In some embodiments, the cell formulation is never frozen. In illustrative embodiments, the cell formulation contains less than, or less than about 7%, about 6%, about 5%, about 4%, about 3%, about 2%, or about 1% DMSO (v/v). In further illustrative embodiments, the cell formulation contains no DMSO. In some illustrative embodiments, the cell formulation is frozen.

A uniform single cell suspension is ideal for intravenous delivery but is not required for subcutaneous or intramuscular administration. In some embodiments, the cell formulation for subcutaneous or intramuscular delivery is a depot formulation or emulsion of cells that promotes cell aggregation, and a delivery solution herein used to prepare such a depot cell formulation, includes the accessory components that provide depot properties. In some embodiments, the cells may be aggregated in the formulation, for example before it is administered to a subject, or for example within 1 hour, 45 minutes, 30 minutes, 15 minutes, 10 minutes, 5 minutes, or 1 minute of cells, for example modified lymphocytes as provided herein, being formulated in a delivery solution, for example comprising an aggregating agent to produce the formulation. In some embodiments, at least 10%, 20%, 25%, 50%, 75%, 90%, 95%, or 99% of the cells in a cell formulation provided herein are aggregated. Such aggregation can be determined, for example, using microscopic counting of individual cells versus cells that are associated with at least one other cell, or by counting the number of cells on average, a cell within a formulation is associated with. In some embodiments the cell formulation is designed for controlled or delayed release with tissue expansion to accommodate cell expansion.

In some embodiments, a delivery solution provided herein, for subcutaneous or intramuscular delivery is a depot formulation. A depot (i.e., sustained release) formulation is typically an aqueous or oleaginous suspension or solution.

Accordingly, in some embodiments, the delivery solution or cell formulation includes components that form an artificial extracellular matrix such as a hydrogel. In some embodiments, a depot delivery solution comprises an effective amount of alginate, collagen, and/or dextran to form a depot formulation. One class of polymers that can be used to make gel-forming biomaterials, and can be included in delivery solutions and cell formulations provided herein, is composed of poly(ethylene glycol) (PEG) and its copolymers with aliphatic polyesters, such as poly(lactic acid) (PLA), poly(D,L-lactic-co-glycolic acid) (PLGA), poly(ε-caprolactone) (PCL) and polyphosphazenes. Other polymers that can be used include thermosensitive triblock copolymers based on poly(N-(2-hydroxypropyl methacrylamide lactate) and poly(ethylenglycol) (p(HPMAm-lac)-PEG), capable of spontaneous self-assembling in physiological environments (Vermonden et. al 2006, Langmuir 22: 10180-10184).

In some embodiments, the hydrogel used in a delivery solution or cell formulation herein, contains hyaluronic acid (HA). Such HA can have carboxylic acid groups that can be modified with 1-ethyl-3-(3-dimethyl aminopropyl)-1-carbodiimide hydrochloride to react with amine groups on proteins, peptides, polymers, and linkers, such as those found on modified lymphocytes provided herein, preferentially in the presence of N-hydroxysuccinimide. Antibodies, cytokines and peptides can be chemically conjugated to HA using such methods to produce a hydrogel for co-injection as a cell emulsion in some cell formulation embodiments provided herein. Additionally, in some embodiments, HA in delivery solutions and cell formulations is a polymer (e.g., Healon) and/or are crosslinked (e.g., restylane (AbbVie/Allergan)), for example lightly crosslinked, through its —OH groups with agents such as glutaraldehyde to reduce the local catabolism of the material following subcutaneous injection. The HA used in delivery solutions and cell formulations herein, can be of variable length and viscosity. The HA used in delivery solutions and cell formulations herein, can further be crosslinked with other glycosaminoglycans such as chondroitin sulfate (e.g., Viscoat) or polymers or surfactants. A skilled artisan will recognize that the porosity of the matrix and degree of crosslinking can be regulated to ensure cells, such as modified lymphocytes herein, are capable of migration through the hydrogels. Accordingly, a matrix, such as a hydrogel matrix, when used in a cell formulation herein, can be configured for, or adapted to permit migration of cells through the matrix. The degree of substitution of the hydrogel and concentration at the time of crosslinking will influence porosity swelling ratio and Youngs Modulus (or stiffness). Initial 1% substitution of HA with tyramine for example at 1 mg/ml when subsequently crosslinked in the presence of peroxide will result in a hydrogel with higher porosity and lower stiffness than 3% substitution and 5 mg/ml solution. Reducing the shear modulus is desirable in some circumstances to reduce shear force during injection and ensure adequate porosity and half life for cells to expand into the matrix subcutaneously over one to two weeks. In some embodiments, the shear modulus is or is about 2.5 kPa, about 3 kPa, about 3.5 kPa, or about 4 kPa.

In some embodiments the delivery solution, a composition in the kit, or the cell formulation includes one or more cytokines such as IL-2, IL-7, IL-15, IL-21, or variants thereof, or an active fragment of any of the preceding and/or cytokine receptor agonists, such as an IL-15 agonist. In some embodiments the delivery solution, a composition in the kit, and/or the cell formulation includes one or more of IL-1, IL-2, IL-7, IL-12, IL-15, IL-18, IL-21, TNFα, IFNγ, GM-CSF, CCL1, CCL2 (MCP-1), CCL3, CCL5, CCL7 (MCP-3), CCL8 (MCP-2), CCL19, CCL20, CCL21, CCL22, CCL28, CXCL1, CXCL9, CXCL10, CXCL11, CXCL12, CXCL14 (BRAK), CX3CL1, and variants thereof, and an active fragment of any of the preceding. In some embodiments, the delivery solution, a composition in the kit, and/or the cell formulation does not include IL-2, IL-7, IL-15, or IL-21. In some embodiments, the delivery solution, a composition in the kit, and/or the cell formulation includes one or more of IL-1, IL-12, IL-18, TNFα, IFNγ, GM-CSF, and variants thereof, and an active fragment of any of the preceding. In some embodiments, the delivery solution, a composition in the kit, and/or the cell formulation includes one or more of CCL1, CCL2 (MCP-1), CCL3, CCL5, CCL7 (MCP-3), CCL8 (MCP-2), CCL19, CCL20, CCL21, CCL22, CCL28, and variants thereof, and an active fragment of any of the preceding. In some embodiments, the delivery solution, a composition in the kit, and/or the cell formulation includes one or more of CCL19, CCL21, and variants thereof, and an active fragment of any of the preceding capable of binding to CCR7 and/or CXCR3. In some embodiments, the delivery solution, a composition in the kit, or the cell formulation includes one or more of CXCL1, CXCL9, CXCL10, CXCL11, CXCL12, CXCL14 (BRAK), and variants thereof, and an active fragment of any of the preceding. In some embodiments, the delivery solution, a composition in the kit, and/or the cell formulation includes one or more of CX3CL1, and variants thereof, and an active fragment of any of the preceding. In some embodiments, the delivery solution, a composition in the kit, and/or the cell formulation includes one or more polypeptides capable of binding to CCR2, CCR4, CCR5, CCR6, CCR7, CCR8, CCR9, CXCR3, CXCR4, CXCR5, CXCR6, and/or Cx3cr1. In some embodiments, the delivery solution, a composition in the kit, and/or the cell formulation includes one or more polypeptides capable of binding to CCR7, CXCR3, CXCR4, and/or CXCR6. In some embodiments, the delivery solution, a composition in the kit, and/or the cell formulation includes one or more polypeptides capable of binding to CCR1, CC42, CCR4, CCR5, CCR6, CCR7, CCR8, CCR9, CXCR3, CXCR4, CXCR5, and/or CXCR6.

In some embodiments the cytokine does not bind to a cytokine receptor included in the delivery solution, kit, or cell formulation, and/or does not bind to a cytokine receptor that is encoded by a polynucleotide in the delivery solution, cell formulation, or kit. In some embodiments, the cytokines can be modified cytokines that, not to be limited by theory, selectively activate complexes that drive proliferation. In illustrative embodiments, the modified cytokine is a modified IL-2, for example, a fusion protein with a circularly-permuted IL-2 with the extracellular domain of IL-2Ra (see, e.g., Lopes et al, J Immunother Cancer 2020 April; 8(1): e000673). In some embodiments, the cytokines, modified cytokines, or cytokine receptor agonists can also be administered in one or administrations separate from the cell formulation, before, contemporaneous to, or after the administration including the delivery solution or cell formulation. In some embodiments, two or more separate administrations can be in escalating doses. In some embodiments, two or more administrations can be at the same dose. In some embodiments, two or more administrations can include the same or different cytokines, modified cytokines, and or cytokine receptor agonists. In some embodiments, the separate administrations can be a series of 2, 3, 4, 5, 6, 7, 8, 9, 10, 11, 12, 13, 14, 15, 16, 17, 18, 19, 20, or 21 administrations. In some embodiments, the separate administrations occur on consecutive days.

In some embodiments the cell formulation includes antibodies or polypeptides that are capable of binding CD2, CD3, CD28, OX40, 4-1BB, ICOS, CD9, CD53, CD63, CD81, and/or CD82. The EDC-NHS reaction may be used for linking such proteins to HA or through other intermediates described above. In some embodiments these cytokines, antibodies, or polypeptides are crosslinked to components of a hydrogel. The hydrogel may be mixed with the cell suspension using a syringe connector and two syringes prior to injection. In other embodiments, these cytokines, antibodies, or polypeptides are in solution. In some embodiments, the delivery solution or the cell formulation includes RNA that encodes for these cytokines, antibodies, or polypeptides.

The proliferation and survival of genetically modified T cells and/or NK cells expressing a CAR are promoted by signaling through the CAR when it binds its cognate antigen in the proper context. In some embodiments, the antigen can be added to or co-administered with modified and/or genetically modified T cells and/or NK cells. In some embodiments the antigen is a protein, a glycoprotein, a carbohydrate or fragment thereof such as a peptide, glycopeptide, or functional group. In some embodiments, the antigen can be soluble. In some embodiments, the antigen is from a non-human source. In some embodiments, the antigen can be immobilized on a surface of the artificial matrix, such as a hydrogel. In some embodiments the antigen is a nucleic acid such as DNA or RNA. In some embodiments, the nucleic acid encodes a protein or peptide antigen that is an antigen recognized by the CAR. In illustrative embodiments, the antigen can be expressed on the surface of a cell comprising the nucleic acid encoding the protein or peptide antigen, such that the cell is a target cell, referred to as feeder cells herein. In some embodiments, such target cells are present in large numbers in whole blood and are naturally present in the cell formulation without having to be added. For example, B cells are present in whole blood, isolated TNCs, and isolated PBMCs and would naturally be present in the cell formulation and could serve as target cells for T cells and/or NK cells expressing a CAR directed to CD19 or CD22, as non-limiting examples which are both expressed on B cells. In other embodiments, such target cells are not present in whole blood or are not present in large numbers in whole blood and thus are added exogenously, for example, feeder cells. In some embodiments, target cells can be isolated or enriched from the subject, such as from a tumor sample, using methods known in the art. In other embodiments, cells from the subject or from a source other than the subject, including cell lines, are modified to express the appropriate antigen. In some embodiments, the targets cells are treated to reduce their proliferative capacity by for example, radiation or chemotherapeutic agents before they are administered to a subject. In illustrative embodiments, the antigen expressed on the target cell can include all or a portion of the protein that contains the antigen. In further illustrative embodiments, the antigen expressed on the target cell can include all or a portion of the extracellular domain of the protein that includes the antigen. In some embodiments, the antigen is an antibody that recognizes the ASTR of the CAR, such as an anti-idiotype antibody directed to the scFv domain of the CAR. In some embodiments, the antigen expressed on the target cell can be a fusion with a transmembrane domain that anchors it to the cell surface. Any of the transmembrane domains disclosed elsewhere herein can be used. In some embodiments, the antigen expressed on the target cell can be a fusion with a stalk domain. Any of the stalk domains disclosed elsewhere herein can be used. In illustrative embodiments, the antigen can be a fusion with a CD8 stalk and transmembrane domain (SEQ ID NO:24).

In illustrative embodiments, cells in a first cell mixture, for example cells obtained from a subject, are modified with a recombinant nucleic acid vector encoding a target antigen, which can be referred to herein as “artificial antigen presenting cells” or “aAPCs”, and cells in a separate second cell mixture from the same subject are modified to express the CAR that binds the antigen. In some embodiments, where the modified cell that was modified with a vector encoding a target antigen is a T cell, the cell can be called a “T-APC” herein. Such modified T-APCs can include, as non-limiting examples, B cells, dendritic cells, and macrophages, and in illustrative embodiments dendritic cells and macrophages such as where a corresponding CAR-T target is a B cell cancer target, and can be generated using methods provided herein where reaction mixtures for modification (e.g., transduction) include a T cell binding polypeptide, such as a polypeptide directed to CD3. In further illustrative embodiments, the cell mixture is whole blood, isolated TNCs, isolated PBMCs. For example, the first cell mixture can be modified with a recombinant nucleic acid vector encoding a fusion protein of the extracellular domain of Her2 and the transmembrane domain of PDGF and the second cell mixture can be modified with a recombinant nucleic acid vector encoding a CAR directed to HER2. The cells can then be formulated into the delivery solution or otherwise administered to the subject at varying CAR effector cell-to-target-cell ratios. In some embodiments, the effector-to-target ratio at the time of formulation or administration is, or is about 10:1, about 9:1, about 8:1, about 7:1, about 6:1, about 5:1, about 4:1, about 3:1, about 2:1, about 1:1, about 1:2, about 1:3, about 1:5, about 1:6, about 1:7, about 1:8, about 1:9, or about 1:10. In illustrative embodiments, target cells are co-administered with the modified T and/or NK cells subcutaneously or intramuscularly.

The proliferation and survival of genetically modified T cells and/or NK cells expressing a CAR can also be promoted by CAR signaling initiated by cross-linking the CARs by interactions other than through the CAR's ASTRs binding to their cognate antigens. In some embodiments, a small molecule or protein can cross-link and activate CARs on the surface of a cell. In illustrative embodiments, an antibody can cross-link and activate CARs on the surface of a cell. In further illustrative embodiments, the antibody recognizes an epitope in the extracellular domain of the CAR, such as in the stalk or spacer domain. In some embodiments, the epitope can be an epitope tag such as His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), HA Tag (YPYDVPDYA; SEQ ID NO:73), RYIRS (SEQ ID NO:79), Phe-His-His-Thr (SEQ ID NO:80), or WEAAAREACCRECCARA (SEQ ID NO:81). In illustrative embodiments the epitope is common to an intracellular antigen that is not reactive to an extracellular receptor. In some embodiments, the epitope tag is the HisX6 tag (SEQ ID NO:77). In some embodiments, the CARs can be cross-linked and activated by adding soluble antibodies that bind the epitope tag. In illustrative embodiments, the CARs can be cross-linked and activated by adding cells, also referred to herein as universal feeder cells, expressing antibodies, or antibody mimetics, that bind the epitope tag. In some embodiments, the antibody or antibody mimetic associates with the cell membrane through a GPI anchor. In illustrative embodiments the antibody or antibody mimetic associates with the cell membrane through a transmembrane domain. In further illustrative embodiments, a stalk or spacer separates the antibody or antibody mimetic, from the transmembrane domain. In some embodiments, the same universal feeder cells, for example, universal feeder cells expressing an anti-HisX6 scFv attached to a CD8a stalk and transmembrane domain, can be used with cells that express CARs with ASTRs that bind to different antigens but that include the HisX6 epitope tag in their stalk. These universal feeder cells can be used with cells expressing different CARs containing a common epitope tag. With universal feeder cells, provided the CARs contain the epitope tag, there is no need to generate different feeder cells that express the cognate antigen for CARs containing different ASTRs. The epitope tag on the cells expressing a CAR will be crosslinked by the universal feeder cells to engage clustering and proliferation of the CAR. For example, the anti-HisX6 universal feeder cells can be used with cells expressing a CAR that binds to Her2 and includes the HisX6 epitope tag and could also be used with cells expressing a CAR that binds to Axl and includes the HisX6 epitope tag. The combination of the universal feeder cell and the CAR can enable CAR-T propagation before the cells engage their cognate antigen. Additionally, if the ASTR of the CAR is microenvironment restricted, the use of the universal feeder cell binding to antigen may enable expansion outside that restrictive environment.

Provided herein in another aspect, is a cell formulation comprising an aggregate(s) of T cells and/or NK cells, wherein the T cells and/or NK cells are modified with a polynucleotide comprising one or more transcriptional units, wherein each of the transcriptional units is operatively linked to a promoter active in T cells and/or NK cells, and wherein the one or more transcriptional units encode a first polypeptide comprising a chimeric antigen receptor (CAR) in a solution, in illustrative embodiments a delivery solution; and further wherein the aggregate comprises at least 4, 5, 6, or 8 T cells and/or NK cells, wherein the cell aggregate is at least 15 μM in its smallest dimension, and/or wherein the cell aggregate is retained by a coarse filter having a diameter of at least 15 μm, or a coarse filter having a diameter of between 15 μm and 60 μm.

### Overview of Embodiments Related to T Cells and NK Cells

The present disclosure overcomes prior art challenges by providing improved methods and compositions for modifying and in illustrative embodiments genetically modifying lymphocytes, for example NK cells and in illustrative embodiments, T cells. Some of the methods and compositions herein, provide simplified and more rapid processes for transducing or transfecting lymphocytes that avoid some steps that require specialized devices. Thus, the methods provide an important step toward democratization of cell therapy methods. Illustrative methods and compositions for modifying lymphocytes, for example NK cells and in illustrative embodiments, T cells, are performed ex vivo or in vivo by direct administration of replication incompetent recombinant retroviral particles (RIPs) to a subject. In some aspects and embodiments herein, methods for modifying T cells and/or NK cells are performed in less time than prior methods, and in fact in some embodiments, provide rapid point of care methods. Furthermore, compositions that have many uses, including their use in these improved methods, are provided, including RIP and/or cell formulation and/or compositions that are adapted for perilymphatic, intranodal or subcutaneous administration. Some of these compositions include modified and in illustrative embodiments genetically modified lymphocytes that have improved proliferative and survival qualities, including in in vivo growth and engraftment and/or in ex vivo and/or in vitro culturing, for example in the absence of growth factors. Such RIP formulations and modified and in illustrative embodiments genetically modified lymphocytes will have utility, for example, as research tools to more easily transduce T cells and NK cells in vivo, ex vivo, or in vitro to understand factors that influence T cell proliferation and survival, and for commercial production, for example for the production of certain factors, such as growth factors and immunomodulatory agents, that can be harvested and tested or used in commercial products. And such RIP formulations and modified and genetically modified lymphocytes having have utility in the treatment of many disorders, such as in immune cell or gene therapy, in illustrative embodiments, for the treatment of hyperproliferative cell disorders such as cancer.

Illustrative methods and compositions for immune cell therapy herein, include, are compatible with, are effective for, and/or are adapted for perilymphatic, subcutaneous, intraperitoneal, or intramuscular delivery and cell and/or RIP formulations for such delivery. Some of these delivery methods and cell formulations (i.e., delivery compositions) promote cell aggregation. Such cell aggregation promotes cell proliferation and survival that in some embodiments is further enhanced by the addition of antigen, growth factors and immunomodulatory agents to the cell formulation or to the site of administration of the cell formulation.

Also provided herein are methods and compositions that overcome the challenges associated with the resistance of CAR therapy by CAR-cancer cells such as loss of target antigen availability (e.g., epitope or antigen masking) through genetic modification of malignant cells.

In Vivo Methods for Genetically Modifying T Cells and/or NK Cells

It is noteworthy that aspects and embodiments provided herein include direct injection of RIPs with membrane-bound hyaluronidase into a subject, wherein T cells and/or NK cells are modified in vivo. It is further noteworthy that although many of the aspects and embodiments provided herein are discussed in terms of a recombinant retroviral particle, it is intended, and a skilled artisan will recognize, that many different recombinant nucleic acid vectors, including but not limited to those provided herein, can be used and/or included in such methods and compositions. Accordingly, in some aspects and embodiments, methods provided herein include directly administering (e.g., delivering, introducing, or injecting) viral particles to a subject. In some aspects and embodiments, provided herein are methods for administering viral particles into a subject, to modify, and in illustrative embodiments, genetically modify and/or transduce T cells and/or NK cells in vivo, i.e., in the subject. In illustrative embodiments, the viral particles are retroviral particles. In further illustrative embodiments, the retroviral particles are replication incompetent recombinant retroviral particles (RIPs). Many of the aspects and embodiments herein refer to RIPs, and a skilled artisan can understand how other viral and/or retroviral particles can be used.

In related aspects and embodiments, RIP formulations, modifying compositions comprising RIPs, delivery solutions comprising RIPs, RIPs for use in a method, methods of making RIP formulations, in vivo compositions comprising RIPs, and in vivo reaction mixtures comprising RIPs are provided herein. In illustrative embodiments, the RIPs include one or more polynucleotides that encode an engineered signaling polypeptide, e.g., a CAR, engineered TCR, and/or a lymphoproliferative element (LE). In certain illustrative embodiments, the LE is a constitutively active LE. In illustrative embodiments, the RIP formulation includes activation elements, either in solution or present on the surface of the RIPs, to facilitate genetic modification of T cells and/or NK cells in vivo. In illustrative embodiments, the RIPs include a binding element and a fusogenic element on the surface of the RIP. In some embodiments, one or both of the binding element and fusogenic element can be a pseudotyping element. In some embodiments, the activation element is a binding element and the fusogenic element is a pseudotyping element.

In one aspect, provided herein is a method for administering replication incompetent recombinant retroviral particles (RIPs) to a subject, said method comprising:
administering a formulation comprising the RIPs to the subject, wherein the RIPs comprise:
   a) an activation element on the surface of the RIPs; and
   b) a polynucleotide comprising one or more transcriptional units, wherein each of the one or more transcriptional units encode one or more engineered signaling polypeptides.

The engineered signaling polypeptide can be any of the engineered signaling polypeptides disclosed elsewhere herein, for example, a CAR, an engineered TCR, and/or a lymphoproliferative element.

In another aspect, provided herein is a method for administering replication incompetent recombinant retroviral particles (RIPs) to a subject, said method comprising:
administering a formulation comprising the RIPs to the subject, wherein the RIPS comprise:
   a) an activation element on the surface of the RIPs; and
   b) a polynucleotide encoding a lymphoproliferative element (LE) and encoding a chimeric antigen receptor (CAR), wherein the LE is constitutively active.

The activation element can be any of the activation elements disclosed elsewhere herein. In some embodiments, the RIPs further comprise a binding element and/or a fusogenic element on the surface of the RIPs. Any of the binding or fusogenic elements disclosed elsewhere herein can be used.

In any of the aspects and embodiments herein that include administering RIPs to a subject, including delivering or introducing RIPs to a subject and/or injecting RIPs into a subject, and including methods of treating a subject herein that include administering RIPs to the subject, the administering can include administering the RIPs intravenously or perivascularly. In illustrative embodiments, the RIPs can be administered perivascularly. Perivascular administration includes intratumoral, intranodal, and perilymphatic administration. Thus, in some embodiments, the RIPs can be administered intratumorally, intranodally, or, in illustrative embodiments, perilymphaticly. Perilymphatic administration includes subcutaneous, interstitial, intraperitoneal, intramuscular, and intradermal administration, and in some embodiments, the RIPs can be administered subcutaneously, interstitially, intraperitoneally, intramuscularly, or intradermally. In some embodiments, the RIPs can be delivered intranodally or subcutaneously. Any formulation herein, including those that comprise RIPs, can also be referred to as a composition, such as a modifying composition if it includes for example, RIPs, or a delivery solution.

Introduction or administration of RIPs into a subject can be by direct delivery into lymph nodes. In illustrative embodiments, the RIPs can be injected into or administered to the inguinal, axillary, and/or cervical lymph nodes. In some embodiments, the lymph nodes into which the RIPs are delivered are lymph node metastases. In some embodiments, the lymph nodes are tumor-draining lymph nodes. In some embodiments, the lymph nodes are not tumor-draining lymph nodes. In some embodiments, the lymph nodes comprise TILs.

RIP formulations herein can include components, and thus can have properties, to improve their effectiveness, for example when introduced to a subject, which in non-limiting examples can be by subcutaneous delivery. In some embodiments, the RIPs are formulated to comprise a means for retaining the RIPs at or near the site of delivery or to be effective for, configured to, and/or adapted for retention near the site of delivery. In some embodiments, the RIPs are formulated such that they are effective for, adapted to and/or configured to retain the RIPs at or near the site of delivery, or have a means to retain the RIPs at or near the site of delivery. In some embodiments, the RIPs delivered subcutaneously comprise a membrane-bound cytokine. In some embodiments, the RIPs delivered subcutaneously comprise a membrane-bound chemokine. In some embodiments, the RIPs are delivered in a depot formulation. In some embodiments, the RIPs are delivered in a hydrogel. Depot formulations and hydrogels are discussed in more detail herein. In some embodiments, RIPs are formulated to comprise a means for inhibiting localized absorption into the blood. In some embodiments, RIPs are formulated such that they are effective for, adapted to and/or configured to inhibit localized absorption into the blood. In some embodiments, RIPs are formulated to comprise a means for entry into the lymphatics. In some embodiments, RIPs effective for, adapted to, and/or configured to enter into the lymphatics. In some embodiments, the RIPs are delivered in a soluble formulation. In some embodiments, the RIPs are formulated with an effective amount of one or more excipients that promote lymphatic absorption (a “lymphagogue”). In some embodiments the lymphagogue is selected from human serum albumin, hyaluronidase, colloids including non-sulfated dextrans, dextrans wherein the molecular weight is greater than 10K, 40K, 100K, 200K, 500K, 1000K, or 2000K kDa, and other agents that decrease the local residence time of the RIPs at the site of delivery (e.g., subcutaneous).

In some embodiments, the RIPs are formulated for delivery in a buffer that includes salts, typically in an effective amount for in vivo delivery. In some embodiments, the formulation comprises phosphate-buffered saline (PBS). In some embodiments the formulation further comprises lactose such as from 0.25% lactose to 10% lactose, for example 0.5% to 10%, 0.5% to 8%, 1% to 8%, 1% to 10%, 2% to 8%, 2% to 6%, 3% to 6%, 3% to 5%, 3.5% to 4.5%, 3.6% to 4.4%, 3.7% to 4.3%, 3.8% to 4.2%, 3.9% to 4.1% about 4% lactose or 4% lactose.

In some embodiments of any of the RIP formulations herein or the cell formulations herein, the formulation has a volume of, and/or the RIP formulation and/or cell formulation delivered to a subject has a volume of, between 0.5 and 25 ml, 0.5 and 20 ml, 0.5 and 10 ml, 0.5 and 7.5 ml, 0.5 and 6 ml, 1.0 and 25 ml, 1.0 and 20 ml, 1.0 and 7.5 ml, 1.0 and 5.0 ml, 1 and 5 ml, 2.5 and 25 ml, 2.5 and 20 ml, 2.5 and 7.5 ml, or 2.5 and 5.0 ml.

In some embodiments, a cell formulation can be combined with a RIP formulation immediately before, or within 1, 2, 3, 4, 5, 6, 7, 8, 9, 10, 15, 20, 25, or 30 minutes of injection and thus some, most, or all cells are not modified, genetically modified or transduced.

In some embodiments, a method for administering RIPs to a subject comprising administering a RIP formulation further comprises administering (sometimes referred to herein as co-administering) a cell formulation (also referred to herein as a cell suspension or cell mixture) to the subject. In some embodiments, the cell formulation comprises whole blood collected from the subject. In some embodiments, the cell formulation comprises neutrophils from the subject. In some embodiments, the cell formulation comprises dendritic cells from the subject. In some embodiments, the cell formulation comprises macrophages from the subject. In some embodiments, the whole blood has been subjected to a PBMC and TNC enrichment procedure and the cell formulation comprises enriched cells. In some embodiments, the cell formulation comprises PBMCs. In illustrative embodiments, the cell formulation administered to the subject comprises T cells and/or NK cells. In some embodiments, the cells, for example PBMCs, in illustrative embodiments are not/have not been exposed to a vector such as a RIP ex vivo, but are or have been exposed to an activation element (e.g., anti-CD3 and/or anti-CD28) before being co-administered, such that they are in activated state when they are co-administered and/or their signaling pathways have been engaged ex vivo such that they will become activated after co-administration to the subject with or without further exposure to an activation element. RIPs that are co-administered with such co-administered cells, and in any embodiments, can have an activation element on their surface and/or can encode an LE. However, in certain illustrative embodiments, RIPs that are co-administered with such activated cells, which in illustrative embodiments have not been exposed to a vector (e.g., RIP) ex vivo, do comprise either or both an activation element on their surface and/or a polynucleotide encoding an LE.

In some embodiments, the cells are modified, i.e, have been contacted with a RIP before administration to the subject. In illustrative embodiments, the cells are unmodified, i.e., have not been contacted with a RIP before administration to the subject.

In some embodiments, the RIP formulation and cell formulation can be administered, delivered, introduced, or injected within 0.5, 1, 2, 3, 4, or 5 cm of each other at a target delivery site of the subject, for example on the surface of the skin of the subject. In some embodiments, the administering the cell formulation to the subject occurs simultaneously or within 1, 2, 3, 4, 5, 10, 15, 30, 45, or 60 minutes or 1, 2, 3, 4, 5, 6, 7, or 8 hours of the administering the RIP formulation to the subject.

In some embodiments, a method for administering RIPs and/or cells to a subject comprises administering at least 0.1 ml, 0.5 ml, 1 ml, 1.5 ml, 2 ml, 2.5 ml, 3 ml, 4 ml, or 5 ml, 10 ml, 15 ml, 20 ml, or 25 ml of any of the formulations disclosed herein that comprise RIPs to the subject. In some embodiments, a method for administering RIPs and/or cells to a subject comprises administering 0.1 to 20 ml of a RIP formulation to the subject. In some embodiments, the method for administering RIPs and/or cells to the subject comprises administering 1 to 10 ml or 1 to 20 ml or 1 to 25 ml of a RIP formulation to the subject. In illustrative embodiments, the method for administering RIPs and/or cells to the subject comprises administering 2 to 3 ml of a RIP formulation. In some embodiments, the method for administering RIPs and/or cells to a subject can include any route of administration as disclosed herein. In illustrative embodiments, the method for administering RIPs and/or cells to a subject can include administering the RIPs and/or cells perilymphatically. In some embodiments, the method for administering RIPs and/or cells to a subject can include either a single dosage or multiple dosages. In some embodiments, the method for administering RIPs and/or cells to a subject can include more than 1, 2, 3, 4, 5, or a greater number of dosages.

In some embodiments, a method for administering RIPs to a subject comprises administering at least 1, 10, 100, 1000, 10⁴, 10¹, 10⁶, 10⁷, 10⁸, 10⁹, 10¹⁰, 10¹¹, 10¹², 10¹³, or 10¹⁴ total transducing units (TUs) to the subject. In some embodiments, the method for administering RIPs to a subject comprises administering 1×10⁹ to 4×10⁹ total TUs to the subject. In some embodiments, the method for administering RIPs to a subject comprises administering 1×10⁶ to 5×10⁹ total TUs to the subject. In some embodiments, the TUs administered can be measured in terms of the weight in kg of the subject, i.e., TUs/kg. In some embodiments, a method for administering RIPs to a subject comprises administering at least 1, 10, 100, 1000, 10⁴, 10⁵, 10⁶, 10⁷, 10⁸, 10⁹, 10¹⁰, 10¹¹, 10¹², 10¹³, or 10¹⁴ TU/kg to the subject. In some embodiments, the method for administering RIPs to a subject comprises administering 1 to 10¹⁴ TU/kg, 10⁵ to 10¹⁴ TU/kg, or 10⁶ to 10¹⁴ TU/kg, or 10³ to 10⁸ TU/kg, or 10⁴ to 10⁷ TU/kg to the subject, wherein kg refers to the weight of the subject. In some embodiments, the TUs administered can be measured in terms of the number of target cells found in 1 ml of blood of the subject, i.e., TUs/target cell/ml blood of the subject.

In some embodiments, a method for administering RIPs to a subject comprises administering 1 ng/kg/day to 500 mg/kg/day to the subject. In some embodiments, a method for administering RIPs to a subject comprises administering 1 ng/kg/day to 100 mg/kg/day for a period of 1, 2, 3, 4, 5, 6, or 7 days or consecutive days, or days in a 1, 2, 3, 4, 5, or 6-month period, such as 1 day per month over a 1, 2, 3, 4, 5, or 6 month period.

In some embodiments, a method for administering RIPs to a subject comprises administering at least 1.0×10⁴, 1.0×10⁵, 1.0×10⁶, or 1.0×10⁷ genome copies (GC) to the subject. In some embodiments, a method for administering RIPs to a subject comprises administering 1.0×10⁴ to 1.0×10¹⁵ GC to the subject. In some embodiments, the method for administering RIPs to a subject comprises administering 1.0×10⁹ to 1.0×10¹⁵ GC to the subject. In some embodiments, the dosage administered can be in terms of GC/kg of the subject. In some embodiments, the method of administering RIPs to a subject comprises administering 1.0×10⁹ to 1.0×10¹⁵ GC/kg to the subject.

In some embodiments, a method for administering RIPs to a subject comprises administering at least 1.0×10⁴, 1.0×10⁵, 1.0×10⁶, or 1.0×10⁷ infectious units to the subject. In some embodiments, a method for administering RIPs to a subject comprises administering 1.0×10⁴ to 1.0×10¹⁵ infectious units to the subject. In some embodiments, the method of administering RIPs to a subject comprises administering 1.0×10⁹ to 1.0×10¹⁵ infectious units/kg to the subject as per the body weight. Infectious unit can be quantified by techniques available in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay. In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID50) method, which determines the dilution of virus at which 50% of the cell cultures are infected and hence, generally, can determine the titer within a certain range, such as one log.

In some embodiments, a method for administering RIPs to a subject comprises administering at least 1.0×10⁴, 1.0×10⁵, 1.0×10⁶, or 1.0×10⁷ plaque forming units (PFU) to the subject. In some embodiments, a method for administering RIPs to a subject comprises administering 1.0×10⁴ to 1.0×10¹⁵ PFU to the subject. In some embodiments, the method for administering RIPs to a subject comprises administering 1.0×10³ to 1.0×10¹⁵ PFU to the subject. In some embodiments, the method of administering RIPs to a subject comprises administering 1.0×10⁹ to 1.0×10¹⁵ PFU/kg to the subject as per the body weight.

In some embodiments, a method for administering RIPs to a subject comprises administering at least 1.0×10³, 1.0×10⁴, 1.0×10⁵, 1.0×10⁶, or 1.0×10⁷ dimming units (DU), as disclosed elsewhere herein, to the subject. In some embodiments, a method for administering RIPs to a subject comprises administering 1.0×10³ to 1.0×10¹⁵ DU to the subject. In some embodiments, a method for administering RIPs to a subject comprises administering 1.0×10³ to 1.0×10⁶ DU to the subject. In some embodiments, the method of administering RIPs to a subject comprises administering 10 to 1.0×10¹³ DU/kg to the subject as per the body weight.

In some embodiments, the amount of RIPs administered to the subject can be such that it prevents producing too many integrations (also referred to as integrants) in an individual cell. In some embodiments, on average 5, 4, or 3 or fewer integrants, measured as lentigenome copies per cellular genome are generated. In illustrative embodiments, on average 3 or fewer integrants are generated. In further illustrative embodiments, on average 2 or fewer integrants are generated.

### Treatment Methods

The present disclosure provides various treatment methods that include administering, delivering, and/or injecting replication incompetent recombinant retroviral particles (RIPs) directly to a subject. In illustrative embodiments, such RIPs have an activation element associated with the surface of the RIPs and include a polynucleotide that encodes one or more of a lymphoproliferative element (LE), an engineered T cell receptor (“engineered TCR”), and a chimeric antigen receptor (CAR). In illustrative embodiments, the LE is a constitutively active LE. In illustrative embodiments, the RIPs encode both an LE and a CAR. Such RIPs are capable of, adapted for, and/or effective for transducing T cells and/or NK cells in vivo, such that the in vivo transduced T cells and NK cells express the LE, engineered TCR, and/or CAR. An engineered TCR or a CAR of the present disclosure, when expressed by and present in a T lymphocyte or an NK cell, can mediate cytotoxicity toward a target cell. Such methods herein typically involve administration of substantially purified or purified RIPs to a subject as provided herein. An engineered TCR or CAR of the present disclosure binds to an antigen present on a target cell, thereby mediating killing of a target cell by a T lymphocyte or an NK cell genetically modified to produce the engineered T cell receptor or CAR. In some embodiments, an engineered T cell receptor or an ASTR of the CAR binds to an antigen present on the surface of a target cell.

The present disclosure, in some aspects provides methods of killing, or inhibiting the growth of, a target cell, that involve contacting a cytotoxic immune effector cell (e.g., a cytotoxic T cell, or an NK cell) that is genetically modified to produce a subject engineered T cell receptor or CAR with a target cell, such that the T lymphocyte or NK cell recognizes an antigen present on the surface of the target cell, and mediates killing of the target cell.

In some aspects, the present disclosure provides a method of treating a disease or disorder in an individual having the disease or disorder, the method including: a. introducing an expression vector including a polynucleotide sequence encoding a CAR and/or an LE (e.g., a constitutively active LE) into a subject to produce a genetically engineered cell, for example, a genetically engineered cytotoxic cell, T cell, and/or NK cell. The present disclosure also provides a method of treating a disease or disorder in an individual having the disease or disorder, the method including: a. introducing an expression vector including a polynucleotide sequence encoding a CAR into peripheral blood cells obtained from the subject to produce a genetically engineered cytotoxic cell; and b. administering the genetically engineered cytotoxic cell to the subject. In some embodiments, the method of treating includes administering both an expression vector (e.g., a RIP formulation) and a genetically engineered cytotoxic cell (e.g., a cell formulation comprising modified, genetically modified, or transduced T cells and/or NK cells). In some embodiments, the method of treating includes administering an expression vector (e.g., a RIP formulation) and unmodified cells (e.g., a cell formulation comprising unmodified cells (e.g., T cells and/or NK cells) that have not been in contact with a RIP). In illustrative embodiments of such embodiments, the unmodified cells can be from the subject. In such embodiments, the unmodified cells from the cell formulation and the cells present in the subject before administration can be modified by the RIPs in the RIP formulation. In some embodiments, the RIP formulation modifies cells in the subject and produces a persistent population of cells in the subject, as disclosed elsewhere herein. In some embodiments, the cells modified by the RIP formulation include the cells from a separately administered cell formulation.

In some embodiments, the RIPs and/or cells administered to a subject for a method of treating can include administration of cytokines, as disclosed elsewhere herein. In some embodiments, the cytokines are part, of or co-administered with, a formulation. In some embodiments, the cytokines can be membrane-bound cytokines on the surface of the RIPs or cells, and in illustrative embodiments, chemokines membrane-bound. Cells administered to a subject including a membrane-bound cytokine would typically come from the recent fusion of the cell with a RIP expressing said membrane-bound cytokine, and not from expression of the membrane-bound cytokine in the cell. In illustrative embodiments, the cytokine or membrane-bound cytokine can include one or more of IL-1, IL-2, IL-7, IL-12, IL-15, IL-18, IL-21, TNFα, IFNγ, GM-CSF, CCL1, CCL2 (MCP-1), CCL3, CCL5, CCL7 (MCP-3), CCL8 (MCP-2), CCL19, CCL20, CCL21, CCL22, CCL28, CXCL1, CXCL9, CXCL10, CXCL11, CXCL12, CXCL14 (BRAK), or CX3CL1, or a variant of any of the preceding, or an active fragment of any of the preceding. In illustrative embodiments, the cytokine or membrane-bound cytokine, and in illustrative embodiments, the chemokine or membrane-bound chemokine can be CCL1, CCL2 (MCP-1), CCL3, CCL5, CCL7 (MCP-3), CCL8 (MCP-2), CCL19, CCL20, CCL21, CCL22, or CCL28, or a variant of any of the preceding, or an active fragment of any of the preceding.

Methods provided herein, such as adoptive cell therapies, methods for producing persistent populations of cells, methods for delivering a formulation (e.g., a modified cell T cell and/or NK cell formulation, a RIP formulation, and/or an unmodified T cell and/or NK cell formulation), etc. as non-limiting examples, are especially adopted for treating cancer. Such cancer can be any type of cancer. For example, such methods can be used for treating a subject who has, or a tumor associated with ovarian cancer, soft tissue sarcoma, peripheral T cell cancer, colorectal cancer, intrahepatic cholangiocarcinoma, glioblastoma, esophageal cancer, cutaneous T cell lymphoma, non-hodgkin lymphoma, urothelial cancer, basal cell carcinoma, epithelioid sarcoma, pancreatic cancer, non-small cell lung carcinoma, hodgkin lymphoma, renal cell carcinoma, mesothelioma, metastatic uveal melanoma, kidney cancer, blood cancer, HER2-expressing cancers, non-melanoma skin cancer, liposarcoma, hepatocellular carcinoma, small lymphocytic lymphoma, prostate cancer, breast cancer, anal cancer, marginal zone lymphoma, cutaneous squamous cell carcinoma, thyroid cancer, medullary thyroid cancer, triple-negative breast cancer, neuroendocrine prostate cancer, bladder cancer, paraganglioma, medulloblastoma, superficial basal cell carcinoma, head and neck squamous cell carcinoma, hematologic malignancies, melanoma, B-cell lymphoma, relapsed/refractory acute myeloid leukemia, angiosarcoma, bone sarcoma, refractory cervical cancer, cholangiocarcinoma, osteosarcoma, biliary tract cancer, castration-resistant prostate cancer, gastroesophageal adenocarcinomas, rhabdomyosarcoma, carcinoma, non-muscle invasive bladder cancer, uveal melanoma, small cell lung cancer, cervical cancer, primary open angle glaucoma, follicular lymphoma, synovial sarcoma, liver cancer, carcinosarcoma, leptomeningeal brain tumors, T-cell lymphoma, lymphoma, small cell lung cancer, mantle cell lymphoma, B-cell malignancies, endometrial cancer, myxoid/round cell liposarcoma, metastatic merkel cell carcinoma, neuroblastoma, chronic lymphocytic leukemia, tenosynovial giant cell tumors, sarcoma, acute myeloid leukemia, skin cancer, nasopharyngeal carcinoma, relapsed/refractory ewing sarcoma, bone cancer, glioma, salivary gland carcinoma, gastric cancer, benign tumor, low-grade serous ovarian cancer, metastatic breast cancer, multiple myeloma, diffuse large B cell lymphoma, relapsed/refractory lymphoma, metastatic colorectal cancer, advanced malignancies, acute lymphoblastic leukemia, mesothelin-expressing solid tumors.

In some embodiments, a cancer cell is from a cancer expressing low levels of PDL-1. In some embodiments, the cancer cell is from a cancer expressing medium levels of PDL-1. In some embodiments, the cancer cell is from a cancer expressing high levels of PDL-1. PDL-1 expression on the surface of target cells is typically measured using immunohistochemistry, and is measured from 0-100% on cancer cells, and is reported as a tumor proportion score (TPS). TPS is typically used to determine if a patient is a candidate for a drug approved for PD-1/PDL-1 immunotherapies. In some embodiments, TPS can be used to determine if a cancer is more amenable to treatment with a cell population of CD56+ and/or CD57+ lymphocytes that are either CD8+ or CD4+. A TPS score of at least 50% can be a high PDL-1 expressing cancer. A TPS score of less than 1% is categorized as being negative for PDL-1 expression. A TPS score of at least 1% can be positive for PDL-1 expression. In some embodiments, a TPS score of between 1 and 25% can be a low PDL-1 expressing cancer. In some embodiments, a TPS score of between 25 and 26% can be a medium PDL-1 expressing cancer. In some embodiments, a cancer can be a low PDL-1 expressing cancer if the TPS is at least 1%, 2%, 3%, 4%, 5%, 6%, 7%, 8%, 9%, 10%, 11%, 12%, 12%, 14%, or 15%. In some embodiments, a cancer can be a medium PDL-1 expressing cancer if the TPS is at least 20%, 25%, 30%, or 35%. In some embodiments, a cancer can be categorized as expressing high levels of PDL-1 if TPS is at least 50%. In some embodiments, the cancer expressing high levels of PDL-1 can include hepatocellular carcinoma, pancreatic cancer, gastric cancer, renal cell carcinoma, esophageal cancer, or ovarian cancer.

In some embodiments, the cancer cell is from any type of cancer that is approved for pembrolizumab. In some embodiments, the cancer approved for pembrolizumab is melanoma, non-small cell lung cancer (NSCLC), head and neck squamous cell cancer (HNSCC), classical Hodgkin Lymphoma (cHL), primary mediastinal B-cell lymphoma (PMBCL), urothelial carcinoma, microsatellite instability-high (MSI-H) or a mismatch repair deficient (dMMR) solid tumor, colon or rectal cancer, gastric or gastroesophageal junction (GEJ) adenocarcinoma, esophageal or certain gastroesophageal junction (GEJ) carcinomas, cervical cancer, hepatocellular carcinoma, Merkel Cell Carcinoma (MCC), renal cell carcinoma (RCC), endometrial carcinoma, cutaneous squamous cell carcinoma (cSCC), or triple-negative breast cancer (TNBC). In some embodiments, the cancer cell is from any type of cancer approved for nivolumab. In some embodiments, the cancer approved for nivolumab is non-small cell lung cancer (NSCLC), melanoma, RCC, HNSCC, hepatocellular carcinoma, urothelial carcinoma, cHL, esophageal squamous cell cancer, malignant pleural mesothelioma, GEJ, or esophageal carcinomas.

In some embodiments, methods herein can be used to treat tumors that express any one or more of the tumor-associated antigens and/or tumor-specific antigens provided herein, and engineered T cell receptors and CARs can be designed to recognize such targets, for example, any of the tumor-associated antigens and/or tumor-specific antigens disclosed elsewhere herein. As non-limiting examples, such tumor associated or tumor specific antigens include blood tumor antigens provided herein elsewhere in this specification, and in some non-limiting embodiments includes the following antigens, most or all of which are believed to be associated with solid tumors: AXL, CD44v6, CAIX, CEA, CD133, Claudin 18.2, c-Met, EGFR, EGFRvIII, Epcam, EphA2, GD2, GPC3, GUCY2C, HER1, HER2, ICAM-1, IL13Rα2, IL11Rα, Kras, Kras G12D, LlCAM, MAGE, MET, Mesothelin, MUC1, MUC16 ecto, Nectin4/FAP, NKG2D, NY-ESO-1, PSCA, ROR-2, WT-1.

In some embodiments, any of the methods provided herein that involve an administering step, can be combined with administration of another cancer therapy, which in certain embodiments, can be a cancer vaccine, for example delivered subcutaneously. In other embodiments and optionally in further combination with cancer vaccine administration, such methods provided herein that include administering genetically modified T cells and/or NK cells into a subject, especially where the subject has, is afflicted with, or is suspected of having cancer, can further include delivering an effective dose of an immune checkpoint inhibitor to the subject. This checkpoint inhibitor delivery can occur before, after, or at the same time as administering the genetically modified T cells and/or NK cells. Immune checkpoint inhibitors are known and various compounds are approved and in clinical development. Check point molecules, many of which are the target of checkpoint inhibitor compounds, include, but not limited to an anti-PD1 antibody.

In some embodiments, any of the methods provided herein that involve an administering step, can be combined with administration of another cancer therapy, which in certain embodiments, can be administering an anti-TIGIT antibody. In certain illustrative embodiments, the RIPs or cells can be administered along with an anti-TIGIT antibody. In illustrative embodiments, an administering step includes administering a modified cell population, wherein the cell population comprises a CAR and a lymphoproliferative element as disclosed herein and the cell population after administration is capable of differentiating into CD56+ and/or CD57+ lymphocytes that are either CD8+ or CD4+, and further administering an anti-TIGIT antibody. In some embodiments, the anti-TIGIT antibody is Tiragolumab, Ociperlimab, Vibostolimab, ASP-8374, JS006 (Junshi Biosciences), or COM902. In some embodiments, the anti-TIGIT antibody blocks the TIGIT-PVR pathway.

In some embodiments, the administering is for treating cancer in the subject, and wherein a tumor in the subject regresses within 60 days, 45 days, 30 days, or 14 days after said administering. In some embodiments, the tumor is a blood cancer, for example DLBCL, that in illustrative examples expresses any of the blood cancer antigens provided herein. In other embodiments, the tumor is a solid tumor that expresses a solid tumor antigen, which in certain illustrative embodiments is a HER2 positive solid tumor, such as, but not limited to, breast cancer. In some embodiments, the administering is for treating cancer in the subject, and wherein the subject experiences stable disease, at least a partial response, or a complete response, in illustrative embodiments by RECIST1.1 criteria, within 90 days, 75 days, 60 days, 45 days, 30 days, or 14 days after said administering. In some embodiments, the tumor shrinks by at least 10%, 20%, 25%, 30%, 50% or more. In some embodiments, a partial response occurs when the sum of tumor lesions reduces by 30% or more and is confirmed at least 4 weeks after the prior scan without the appearance of new lesions and/or any pathological lymph nodes have a reduction in short axis to less than 10 mm. In some embodiments, a complete response occurs when all target and non-target lesions disappear. In some embodiments, the administering is for treating cancer in the subject, and wherein the subject experiences at least a partial response or experiences a complete response within 60 days, 45 days, 30 days, or 14 days after said administering. In some embodiments, the subject is a human afflicted with cancer. In some embodiments, the cell formulation is administered 2, 3, 4, 5, 6, or more times, or in illustrative embodiments only once to the subject before stable disease, or in illustrative embodiments a partial response or a complete response is achieved. In some embodiments, a second formulation is administered to the subject at a second, third, fourth, etc. timepoint between 1 day and 1 month, 2 months, 3 months, 6 months, or 12 months after the administering a first cell formulation, wherein the second formulation can be identical to the first formulation, or can comprises any of the formulations provided herein.

In any of the aspects provided herein that include intramuscular, and in illustrative embodiments subcutaneous administration, of RIPs and/or modified and/or or unmodified lymphocytes (e.g., modified T cells and/or NK cells), in certain embodiments, administration by any route provided herein, is performed on a mammalian subject that has been subjected to a lymphodepletion process, as are known in the art. However, in illustrative embodiments, the administration of the modified T cells and/or NK cells, or RIPs, is performed in a method that does not require lymphodepletion of the subject for successful engraftment in the subject and/or for successful reduction of tumor volume in the subject, or that is performed on a mammalian (e.g., human) subject that has not been subjected to lymphodepletion in the prior days, weeks, or months or ever before such administration (e.g., subcutaneous administration). In certain embodiments, the administration is performed on a mammalian (e.g., human) subject that is not suffering from a low white blood cell count, lymphopenia or lymphocytopenia. In certain embodiments, the subcutaneous administration is performed on a subject having a lymphocyte count in the normal range (i.e., 1,000 and 4,800 lymphocytes in 1 microliter (μL) of blood). In certain embodiments, the subcutaneous administration is performed on a subject having between 1,000 and 5,000, over 300, over 500, over 1,000, over 1,500, or over 2,000 lymphocytes per μL of blood). In certain embodiments, the administration, for example subcutaneous administration, is performed on a mammalian (e.g., human) subject that is lymphoreplete, that has an effective number of lymphocytes, that has at least 50%, 60%, 75%, 80%, or 90% of the normal number of lymphocytes in a healthy human, and/or having more than 10, 20, 30, 40, 50, 60, 70, 75, 80, 85, 90, or 95% of their lymphocytes before such administration.

### Membrane-Bound Cytokines

Some embodiments of the method and composition aspects provided herein, include a membrane-bound cytokine typically associated with and/or on the outer membrane or surface of a RIP or on the plasma membrane of a modified, genetically modified and/or transduced T cell and/or NK cell, or population thereof, or polynucleotides encoding a membrane-bound cytokine, in illustrative embodiments within a RIP, for example in the genome of a RIP, or a population thereof, or within a T cell and/or NK cell, or a population thereof. Cytokines are typically, but not always, secreted proteins. Cytokines that are naturally secreted can be engineered as fusion proteins to be membrane-bound. Membrane-bound cytokine fusion polypeptides are included in methods and compositions disclosed herein, and are also an aspect of the invention. In some embodiments, RIPs have a membrane-bound cytokine fusion polypeptide on their surface that is capable of binding a T cell and/or NK cell and promoting proliferation and/or survival thereof, or capable of attracting or retaining an immune cell. Such RIPs can be used, for example, in RIP formulations provided herein for administration to a subject. Typically, membrane-bound polypeptides are incorporated into the membranes of replication incompetent recombinant retroviral particles, and when a cell is transduced by the replication incompetent recombinant retroviral particles, the fusion of the retroviral and host cell membranes results in the polypeptide, such as the cytokine fusion polypeptide, being bound to the membrane of the transduced cell. In some embodiments, RIP formulations and/or a delivery solution comprises membrane-bound cytokine associated with the surface of RIP. In some embodiments, the RIP formulation or a delivery solution including the RIP having membrane-bound cytokine associated with the surface as disclosed herein is administered in vivo to a subject in need thereof. In some embodiments, RIP having membrane-bound cytokine associated with the surface as disclosed herein is contacted to T cells and/or NK cells in vivo (direct RIP administration). In some embodiments, RIP having membrane-bound cytokine associated with the surface as disclosed herein is contacted to T cells and/or NK cells, ex vivo.

In some embodiments, the cytokine fusion polypeptide includes one or more of IL-1, IL-2, IL-7, IL-12, IL-15, IL-18, IL-21, TNFα, IFNγ, GM-CSF, CCL1, CCL2 (MCP-1), CCL3, CCL5, CCL7 (MCP-3), CCL8 (MCP-2), CCL19, CCL20, CCL21, CCL22, CCL28, CXCL1, CXCL9, CXCL10, CXCL11, CXCL12, CXCL14 (BRAK), CX3CL1, or variants thereof, or an active fragment of any of the preceding. In some embodiments, the cytokine fusion polypeptide does not include IL-2, IL-7, or IL-15.

In some embodiments, the cytokine fusion polypeptide comprises a cytokine capable of promoting T and/or NK cell proliferation and/or survival. In some embodiments, the cytokine fusion polypeptide capable of promoting T and/or NK cell proliferation and/or survival includes one or more of IL-2, IL-7, IL-15, IL-21, or variants thereof, or an active fragment of any of the preceding.

In some embodiments, the cytokine fusion polypeptide comprises a cytokine capable of stimulating inflammation. In some embodiments, the cytokine fusion polypeptide includes one or more of IL-1, IL-12, IL-18, TNFα, IFNγ, GM-CSF, or variants thereof, or an active fragment of any of the preceding.

In some embodiments, the cytokine fusion polypeptide comprises a chemotactic cytokine (e.g., chemokine). Not to be limited by theory, a chemotactic cytokine is capable of attracting and optionally retaining an immune cell (for example, a T cell, NK cell, NK T cell, TIL (tumor-infiltrating T cell), MIL (marrow-infiltrating lymphocyte), TINK (tumor-infiltrating NK cell), or dendritic cell). In some embodiments, the chemotactic cytokine comprises a C—C motif. In some embodiments, the chemotactic cytokine comprising a C—C motif comprises one or more of CCL1, CCL2 (MCP-1), CCL3, CCL5, CCL7 (MCP-3), CCL8 (MCP-2), CCL19, CCL20, CCL21, CCL22, CCL28, or variants thereof, or an active fragment of any of the preceding. In illustrative embodiments, the chemotactic cytokine comprising a C—C motif comprises one or more of CCL19, CCL21, or variants thereof, or an active fragment of any of the preceding capable of binding to CCR7 or CXCR3. In some embodiments, the chemotactic cytokine comprises a C-X-C motif. In some embodiments, the chemotactic cytokine comprising a C-X-C motif comprises one or more of CXCL1, CXCL9, CXCL10, CXCL11, CXCL12, CXCL14 (BRAK), or variants thereof, or an active fragment of any of the preceding. In some embodiments, the chemotactic cytokine comprises a C-X3-C motif. In some embodiments, the chemotactic cytokine comprising a C-X3-C motif comprises one or more of CX3CL1, or variants thereof, or an active fragment of any of the preceding.

In some embodiments, the cytokine fusion polypeptide comprises one or more polypeptides capable of binding to CCR2, CCR4, CCR5, CCR6, CCR7, CCR8, CCR9, CXCR3, CXCR4, CXCR5, CXCR6, or Cx3cr1. In illustrative embodiments, the cytokine fusion polypeptide comprises one or more polypeptides capable of binding to CCR7, CXCR3, CXCR4, or CXCR6.

In some embodiments, the cytokine fusion polypeptide comprises one or more polypeptides capable of binding to CCR1, CC42, CCR4, CCR5, CCR6, CCR7, CCR8, CCR9, CXCR3, CXCR4, CXCR5, or CXCR6.

The membrane-bound cytokine fusion polypeptides are typically a cytokine fused to heterologous signal sequence and/or a heterologous membrane attachment sequence. In some embodiments, the heterologous membrane attachment sequence is a GPI anchor attachment sequence. The heterologous GPI anchor attachment sequence can be derived from any known GPI-anchored protein as disclosed elsewhere herein. In some embodiments, the membrane-bound cytokine fusion polypeptide comprises a cleavage site. In some embodiments, the cleavage site can be within the sequence of the cytokine. In some embodiments, the cleavage site can be within the sequence of the heterologous signal sequence. In some embodiments, the cleavage site can be within the sequence of the heterologous membrane attachment sequence. In some embodiments, the cleavage site can be between the cytokine and the heterologous signal sequence or the heterologous membrane attachment sequence.

In some embodiments, the membrane-bound cytokine fusion polypeptide can include linkers as disclosed elsewhere herein.

### Targeting Elements

Many of the methods, compositions, and kits provided herein include viral particles, and in illustrative embodiments retroviral particles, that include multiple copies of one or more targeting elements on their surfaces. A “targeting element” can include one or more polypeptides that target, e.g., bind, the target cell that the virus will transduce. In some embodiments, a targeting element can be a binding element or polypeptide. In some embodiments, a targeting element can be an activation element, and will target the target cell in addition to activating the target cell. In some embodiments, the targeting element can be an anti-idiotype polypeptide, which in illustrative embodiments bind to an FDA-approved antibody. In some embodiments, a targeting element can be a binding element, an activation element, and/or an anti-idiotype polypeptide. The targeting element does not comprise both an ASTR and an intracellular activating domain (e.g., the targeting element is not a CAR or TCR as disclosed elsewhere herein).

Typically, one or more cell types are targeted for viral binding by including one or more targeting elements on the surface of the viral particle that can bind to a molecule on the target cell. Thus, in some embodiments, the targeting element can bind to a molecule on the surface of a target cell. In some embodiments, the targeting element can bind to 5T4, αvβ6 integrin, alpha-fetoprotein (AFP), an abnormal p53 protein, an abnormal ras protein, ASGPR, ASGPR 1, ASGPR1, Asialoglycoprotein Receptor 1 (ASGR1), Axl, B cell maturation antigen (BCMA), B7-H3, B7-H6, CAi9-9, CAIX, calretinin, cancer antigen-125 (CA-125), carcinoembryonic antigen (CEA), CCR5, CCR7, CD104, CD105, CD117, CD11b, CD11c, CD123, CD133, CD138, CD14, CD140b, CD146, CD16, CD171, CD184, CD19, CD20, CD21, CD22, CD23, CD24, CD25, CD27, CD28, CD3, CD30, CD31, CD33, CD34, CD37, CD38, CD4, CD40, CD44, CD44v6, CD44v7/8, CD45, CD45RA, CD45RO, CD5, CD55 (DAF1), CD56, CD57, CD62L, CD7, CD70, CD8, CD86, CD90, CD99, chromogranin, CK18, c-kit, c-met, cone opsin, CS1, CS-1, CSPG4, C-Type Lectin Domain Family 4 Member H1 (CLEC4H1), CXCR4, DC-SIGN (also known as CD209), desmin, EGFR, EGFRvIII (epidermal growth factor variant III), EGP2, EGP40, Encephalopsin (OPN3), EpCAM, EphA2, Eph-B2, epithelial membrane protein (EMA), epithelial tumor antigen (ETA), FAP (Fibroblast Activation Protein), fetal AchR, FoxP3, FRα, galactin, GD2, GD2 (ganglioside G2), GD3, GPC3, GUCY2C, Hepatic Lectin H1 (HL-1), HER1, Her2 (ERBB2), high molecular weight-melanoma associated antigen (HMW-MAA), HLA-A1+MAGE1, HLA-A1+NY-ESO-1, HLA-DR, ICAM-1, IL11Rα, IL-11Rα, IL13Rα2, IL-13Rα2, integrin αvβ3 (CD61), integrins, Itm2a, kappa, KIRs, Kras, K-Ras (V-Ki-ras2 Kirsten rat sarcoma viral oncogene), Kras G12D, Krt8, LICAM, lambda, Lewis-Y, MAGE-A1, melanoma-associated antigen (MAGE), mesothelin, MET, motor neuron marker non-phosphorylated neurofilament-H (SMI-32), MUC1, MUC-1, Muc16, MUC16 ecto, muscle-specific actin (MSA), MyoD, myo-D1, myogenin, NCAM, neurofilament, neuron-specific enolase (NSE), New York esophageal squamous cell carcinoma antigen (NYESO1), NKG2D, NKG2D Ligands, NY-ESO-1, PAP (prostatic acid phosphatase), Pax7, PDL-1, placental alkaline phosphatase, PRAME, prostate stem cell antigen (PSCA), prostein, protein melan-A (melanoma antigen recognized by T lymphocytes; MART-1), PSCA, PSMA (prostate-specific membrane antigen), Ral-B, recoverin, rhodopsin, Rlbp1, ROR1, ROR2, Rrh, Silv (also known as Pmel17), sperm protein 17 (Sp17), STEAP1 (six-transmembrane epithelial antigen of the prostate 1), Survivin, synaptophysin, TAG72, TARP (T cell receptor gamma alternate reading frame protein), TCR, TEMs, the dimeric form of the pyruvate kinase isoenzyme type M2 (tumor M2-PK), thyroglobulin, thyroid transcription factor-1, Trp-p8, tyrosinase, Vap2, VEGFR2, or WT-1.

In some embodiments, T cells can be targeted by including one or more targeting elements on the surface of the viral particle that can bind to CD3, CD4, CD5, and/or CD8. In some embodiments, B cells can be targeted by including one or more targeting elements on the surface of the viral particle that can bind to CD19, CD20, and/or CD21. NK cells can be targeted by including one or more targeting elements on the surface of the viral particle that can bind to CS-1, CD16 and/or CD56. In some embodiments, dendritic cells can be targeted by including one or more targeting elements on the surface of the viral particle that can bind to DC-SIGN (also known as CD209), CD11c, and/or CD86. In some embodiments, muscular vascular endothelial cell can be targeted, including by including one more targeting elements on the surface of the viral particle that can bind to CD31, CD146, c-met, Eph-B2, and/or Vap2. In some embodiments, macrophages can be targeted by including one or more targeting elements on the surface of the viral particle that can bind to CD11b and/or CD14. In some embodiments, neutrophils can be targeted by including one or more targeting elements on the surface of the viral particle that can bind to CD11b and/or CD16. In some embodiments, hematopoietic stem cells can be targeted by including one or more targeting elements on the surface of the viral particle that can bind to CD34. In some embodiments, bone marrow stem cells can be targeted by including one or more targeting elements on the surface of the viral particle that can bind to CD34, CXCR4, c-kit, and/or integrins. In some embodiments, endothelial cells can be targeted by including one or more targeting elements on the surface of the viral particle that can bind to c-met, CD31 and/or CD146. In some embodiments, epithelial cells can be targeted by including one or more targeting elements on the surface of the viral particle that can bind to EpCAM, and/or CK18. In some embodiments, fibroblasts can be targeted by including one or more targeting elements on the surface of the viral particle that can bind to CD90 and/or CD105. In some embodiments, retinal cells can be targeted by including one or more targeting elements on the surface of the viral particle that can bind to recoverin, rhodopsin, and/or cone opsin. In some embodiments, retinal pigment epithelium cells can be targeted by including one or more targeting elements on the surface of the viral particle that can bind to CD140b, CD56, CD104, CD184, GD2, Rlbp1, Krt8, Silv (also known as Pmel17), and/or Rrh. In some embodiments, the one or more targeting elements can bind to CD140b, CD56, CD104, CD184, and/or GD2. In some embodiments, the one or more targeting elements can bind to Rlbp1, Krt8, Silv (also known as Pmel17), and/or Rrh. In some embodiments, motor neurons can be targeted by including one or more targeting elements on the surface of the viral particle that can bind to motor neuron marker non-phosphorylated neurofilament-H (SMI-32), Encephalopsin (OPN3), Itm2a, and/or CD55 (DAF1). In some embodiments, skeletal muscle cells can be targeted by including one or more targeting elements on the surface of the viral particle that can bind to desmin, myogenin and/or CD56 with CD34, Pax7, and/or MyoD. In some embodiments, hepatocytes can be targeted by including one or more targeting elements on the surface of the viral particle that can bind to Asialoglycoprotein Receptor 1 (ASGR1, also known as ASGP-R 1, ASGPR 1, ASGPR1, ASGPR, C-Type Lectin Domain Family 4 Member H1 (CLEC4H1), Hepatic Lectin H1 (HL-1)).

In some embodiments, the targeting element can bind to CCR5, CD4, CD8, CD16, CD25, CD27, CD28, CD44, CD45RA, CD45RO, CD56, CD57, CD62L, CCR7, KIRs, FoxP3, and/or TCR components such as CD3.

In some embodiments, the targeting element can bind to a molecule typically associated with a cancer, e.g., a tumor associated antigen on the surface of a target cell. In some embodiments, the targeting element can bind to Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD5, CD7, CD19, CD20, CD22, CD23, CD24, CD27, CD30, CD33, CD34, CD37, CD38, CD40, CD44, CD44v6, CD44v7/8, CD45, CD70, CD99, CD117, CD123, CD133, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, c-Met, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), CS1, PDL-1, GPC3, GUCY2C, HER1, ICAM-1, IL13Rα2, IL11Rα, Kras, Kras G12D, LiCAM, MET, MUC1, MUC16 ecto, NKG2D, NY-ESO-1, PSCA, WT-1 and the like.

In some embodiments, the targeting element can be an antibody. In some embodiments, the targeting element comprises an alternative non-antibody scaffold, also referred to herein as an antibody mimetic. In any of the aspects or embodiments provided herein that include a targeting element capable of binding to a target cell, the targeting element can be an antibody mimetic. In some embodiments, the antibody mimetic can be an affibody, an afflilin, an affimer, an affitin, an alphabody, an alphamab, an anticalin, a VHH, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain. In any of the aspects or embodiments provided herein that include a targeting element that is an antibody, a suitable antibody mimetic can be used instead of the antibody. In some embodiments, the targeting element can be a cytokine. In some embodiments, the targeting element can be any of the cytokines, chemokines, ligands, or receptors disclosed elsewhere herein.

In some embodiments, the targeting element (e.g., binding element) on the surface of a viral particle can be an anti-idiotype polypeptide, as disclosed elsewhere herein. In such embodiments, a target cell that the viral particle will bind to can be marked with an antibody, and the anti-idiotype polypeptide on the surface of the viral particle can bind the antibody on the surface of the cell. The anti-idiotype polypeptide can be expressed in a packaging cell such that the viral particle includes the polypeptide on its surface. In some embodiments, the viral particle does not include a polynucleotide that encodes an anti-idiotype polypeptide. In other embodiments, the viral particle includes a polynucleotide that encodes the anti-idiotype polypeptide. In such embodiments, the antibody can be any FDA-approved antibody.

### Absolute Numbers of Targeting Elements on the Surface of a Viral Particle

The absolute number of individual targeting elements on the surface of a viral particle, and in illustrative embodiments a retroviral particle, can affect how the viral particle interacts with its target cell. Not to be limited by theory, higher absolute numbers of targeting elements on the surface of a viral particle can increase the probability that the viral particle will bind to the target cell and/or can result in multiple binding events on one cell and tighter binding overall. Some target cells and/or targeting elements may require fewer or more targeting elements on the surface of the viral particle. Thus, the absolute number of targeting elements on the surface of a viral particle can be one factor in determining the binding of the targeting element to the target cell.

In some embodiments, each viral particle, and in illustrative embodiments a retroviral particle, can include at least 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle. In some embodiments, each viral particle can include no more than 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, or 4,500 targeting elements on the surface of one retroviral particle on the low end of the range and 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle on the high end of the range.

In some embodiments, between 100 targeting elements on the surface of one retroviral particle on the low end of the range and 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle on the high end of the range. In some embodiments, between 500 targeting elements on the surface of one retroviral particle on the low end of the range and 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle on the high end of the range. In some embodiments, between 1,000 targeting elements on the surface of one retroviral particle on the low end of the range and 1,100, 1,200, 1,300, 1,400, 1,500, 1,600, 1,700, 1,800, 1,900, 2,000, 2,500, 3,000, 3,500, 4,000, 4,500, or 5,000 targeting elements on the surface of one retroviral particle on the high end of the range. In some embodiments, between 2,000 targeting elements on the surface of one retroviral particle on the low end of the range and 2,500, 3,000, 3,500, 4,000, 4,500, or 5,000 targeting elements on the surface of one retroviral particle on the high end of the range.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, or 4,500 targeting elements on the surface of one retroviral particle on the low end of the range and 5,000 targeting elements on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, 3,000, or 3,500 targeting elements on the surface of one retroviral particle on the low end of the range and 4,000 targeting elements on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, 1,900, 2,000, or 2,500 targeting elements on the surface of one retroviral particle on the low end of the range and 3,000 targeting elements on the surface of one retroviral particle on the high end of the range. In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, 1,000, 1,100, 1,200, 1,300, 1,400, 1,500, 1,600, 1,700, 1,800, or 1,900 targeting elements on the surface of one retroviral particle on the low end of the range and 2,000 targeting elements on the surface of one retroviral particle on the high end of the range.

In some embodiments, each viral particle can include between 1, 2, 3, 4, 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, or 900 targeting elements on the surface of one retroviral particle on the low end of the range and 1,000 targeting elements on the surface of one retroviral particle on the high end of the range.

In some embodiments, each viral particle can include between 50 and 5,000 targeting elements on the surface of one retroviral particle. In some embodiments, each viral particle can include between 500 and 4,500 targeting elements on the surface of one retroviral particle. In some embodiments, each viral particle can include between 2,000 and 4,000 targeting elements on the surface of one retroviral particle. In some embodiments, each viral particle can include between 2,500 and 3,500 targeting elements on the surface of one retroviral particle.

### Ratios of Hyaluronidase Polypeptides to Targeting Elements on the Surface of a Viral Particle

In any of the aspects and embodiments herein that include a viral particle with a membrane-bound hyaluronidase, the viral particle can further include a targeting element. The ratio of the absolute number of hyaluronidase polypeptides to the absolute number of targeting elements on the surface of a viral particle, and in illustrative embodiments retroviral particle, can affect how the viral particle moves within the extracellular matrix (ECM) and how it interacts with the target cell. Not to be limited by theory, relatively more hyaluronidase polypeptides can increase the degradation of the ECM, and result in a viral particle that moves more easily through the ECM. Not to be limited by theory, relatively more targeting elements can increase the binding of the viral particle to the target cell. Thus, in some embodiments, a packaging cell is engineered to generate viral particles with the desired ratio of hyaluronidase polypeptides to targeting elements on the surface of the viral particles. For example, the relative expression of hyaluronidase polypeptides and targeting elements in the packaging cell can be engineered to express more or less of one or the other, or the hyaluronidase polypeptides or targeting elements can be engineered to associate or not associate with other viral proteins to increase or decrease the number of hyaluronidase or targeting elements in the viral particles.

In some embodiments, the ratio of hyaluronidase polypeptides to targeting elements on the surface of a viral particle, and in illustrative embodiments retroviral particle, can be at least 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1. In some embodiments, the ratio of hyaluronidase polypeptides to targeting elements on the surface of a retroviral particle can be no more than 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1.

In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, or 1,000:1 on the low end of the range and 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range.

In some embodiments, the ratio of hyaluronidase polypeptides to targeting elements on the surface of a retroviral particle can be between 1:2,000 on the low end of the range and 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range. In some embodiments, the ratio of hyaluronidase polypeptides to targeting elements on the surface of a retroviral particle can be between 1:1,000 on the low end of the range and 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range. In some embodiments, the ratio of hyaluronidase polypeptides to targeting elements on the surface of a retroviral particle can be between 1:500 on the low end of the range and 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range. In some embodiments, the ratio of hyaluronidase polypeptides to targeting elements on the surface of a retroviral particle can be between 1:100 on the low end of the range and 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range. In some embodiments, the ratio of hyaluronidase polypeptides to targeting elements on the surface of a retroviral particle can be between 1:10 on the low end of the range and 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range. In some embodiments, the ratio of hyaluronidase polypeptides to targeting elements on the surface of a retroviral particle can be between 1:1 on the low end of the range and 2:1, 1:1, 4:1, 5:1, 10:1, 20:1, 30:1, 40:1, 50:1, 75:1, 100:1, 150:1, 200:1, 250:1, 500:1, 1,000:1, or 2,000:1 on the high end of the range.

In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, 500:1, or 1,000:1 on the low end of the range and 2,000:1 on the high end of the range. In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, 250:1, or 500:1 on the low end of the range and 1,000:1 on the high end of the range. In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, 75:1, 100:1, 150:1, 200:1, or 250:1 on the low end of the range and 500:1 on the high end of the range. In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, 5:1, 10:1, 20:1, 30:1, 40:1, 50:1, or 75:1 on the low end of the range and 100:1 on the high end of the range. In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, 1:2, 1:1, 2:1, 1:1, 4:1, or 5:1 on the low end of the range and 10:1 on the high end of the range. In some embodiments, the ratio of hyaluronidase polypeptides to targeting elements on the surface of a retroviral particle can be between 1:2,000, 1:1,000, 1:500, 1:250, 1:200, 1:150, 1:100, 1:75, 1:50, 1:40, 1:30, 1:20, 1:10, 1:5, 1:4, 1:3, or 1:2 on the low end of the range and 1:1 on the high end of the range.

In some embodiments, the ratio of hyaluronidase polypeptides to targeting elements on the surface of a retroviral particle can be between 1:2,000 and 2,000:1. In some embodiments, the ratio of hyaluronidase polypeptides to targeting elements on the surface of a retroviral particle can be between 1:1,000 and 1,000:1. In some embodiments, the ratio of hyaluronidase polypeptides to targeting elements on the surface of a retroviral particle can be between 1:500 and 500:1. In some embodiments, the ratio of hyaluronidase polypeptides to targeting elements on the surface of a retroviral particle can be between 1:100 and 100:1. In some embodiments, the ratio of hyaluronidase polypeptides to targeting elements on the surface of a retroviral particle can be between 1:10 and 10:1. In some embodiments, the ratio of hyaluronidase polypeptides to targeting elements on the surface of a retroviral particle can be between 1:100 and 1:1. In some embodiments, the ratio of hyaluronidase polypeptides to targeting elements on the surface of a retroviral particle can be between 1:100 and 1:10. In some embodiments, the ratio of hyaluronidase polypeptides to targeting elements on the surface of a retroviral particle can be between 1:10 and 1:1.

### Binding and Fusogenic Elements

Many of the methods, compositions, and kits provided herein include viral particles, and in illustrative embodiments retroviral particles, with envelope proteins on their surface, for example, multiple copies of a T cell and/or NK cell binding polypeptide and multiple copies of a fusogenic polypeptide, also called a fusogen. A “binding polypeptide” or “binding element” includes one or more polypeptides, typically glycoproteins, that bind the target host cell. In some embodiments, a targeting element can be the binding element. In some embodiments, an activation element can be the binding element. A “fusogenic polypeptide” or “fusogenic element” mediates fusion of the retroviral and target host cell membranes, thereby allowing a retroviral genome to enter the target host cell. In certain embodiments, the binding polypeptide(s) and the fusogenic polypeptide(s) are on the same envelope protein, e.g., a heterologous glycoprotein. In other embodiments, the binding polypeptide(s) and the fusogenic polypeptide(s) are on two or more different heterologous glycoproteins. In some embodiments, the binding element and/or fusogenic element are viral envelope proteins. In some embodiments, the binding element and/or fusogenic element are not viral envelope proteins. In some embodiments, the binding element and/or fusogenic element are two or more different polypeptides.

One or both of these binding and fusogenic polypeptide functions can be provided by a viral envelope protein. In some embodiments, the viral envelope protein can be one or more viral envelope proteins. In some embodiments, the binding polypeptide function of a viral envelope protein can be altered, reduced, or eliminated (e.g., the amino acids corresponding to the binding polypeptide function can be mutated or deleted). In some embodiments, the viral envelope protein with reduced or eliminated binding polypeptide function can be retargeted with a new binding polypeptide function or by mutating the original binding polypeptide function.

In some embodiments, the binding polypeptide function can be provided by any polypeptide that binds to a cell surface marker on the target cell. For example, the binding polypeptide function can be provided by a targeting element or an activation element, as disclosed elsewhere herein. The pseudotyping of replication incompetent recombinant retroviral particles with viral envelope proteins, e.g., heterologous envelope glycoproteins, typically alters the tropism of a virus and facilitates the transduction of host cells. In some embodiments provided herein, viral envelope proteins are pseudotyping elements and are provided as polypeptide(s)/protein(s), or as nucleic acid sequences encoding the polypeptide(s)/protein(s).

In some embodiments, a viral envelope protein (e.g., a pseudotyping element and/or a fusogenic envelope protein) herein is a full-length polypeptide(s), functional fragment(s), homolog(s), or functional variant(s) of a syncytium-inducing polypeptide. In some embodiments, a viral envelope protein herein is a full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezuelan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G. In some embodiments, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

In some embodiments, a viral envelope protein, (e.g., a pseudotyping element and/or a fusogenic envelope protein) herein is a full-length polypeptide(s), functional fragment(s), homolog(s), or functional variant(s) of type G membrane glycoprotein of rabies virus, type G membrane glycoprotein of Mokola virus, type G membrane glycoprotein of vesicular stomatitis virus, type G membrane glycoprotein of Togaviruses, murine hepatitis virus JHM surface projection protein, porcine respiratory coronavirus spike glycoprotein, porcine respiratory coronavirus membrane glycoprotein, avian infectious bronchitis spike glycoprotein and its precursor, bovine enteric coronavirus spike protein, paramyxovirus SV5 F protein, Measles virus F protein, canine distemper virus F protein, Newcastle disease virus F protein, human parainfluenza virus 3 F protein, simian virus 41 F protein, Sendai virus F protein, human respiratory syncytial virus F protein, Measles virus hemagglutinin, simian virus 41 hemagglutinin neuraminidase proteins, human parainfluenza virus type 3 hemagglutinin neuraminidase, Newcastle disease virus hemagglutinin neuraminidase, human herpesvirus 1 gH, simian varicella virus gH, human herpesvirus gB proteins, bovine herpesvirus gB proteins, cercopithecine herpesvirus gB proteins, Friend murine leukemia virus envelope glycoprotein, Mason Pfizer monkey virus envelope glycoprotein, HIV envelope glycoprotein, influenza virus hemagglutinin, poxvirus membrane glycoproteins, mumps virus hemagglutinin neuraminidase, mumps virus glycoproteins FI and F2, West Nile virus membrane glycoprotein, herpes simplex virus membrane glycoprotein, Russian Far East encephalitis virus membrane glycoprotein, Venezuelan equine encephalitis virus membrane glycoprotein, and varicella virus membrane glycoprotein.

In some embodiments, a viral envelope protein that can be employed include, but are not limited to, a full-length polypeptide(s), functional fragment(s), homolog(s), or functional variant(s) of: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Caliciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxviridae, and Iridoviridae) may be utilized. Representative examples include, FeLV, VEE, HFVW, WDSV, SFV, Rabies, ALV, BIV, BLV, EBV, CAEV, SNV, ChTLV, STLV, MPMV, SMRV, RAV, FuSV, MH2, AEV, AMV, CT10, and EIAV.

In some embodiments, pseudotyping elements and/or fusogenic envelope proteins include, but are not limited to a full-length polypeptide(s), functional fragment(s), homolog(s), or functional variant(s) of fusogenic envelope proteins from any of the following sources: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kyasanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephalitis causing virus.

In some embodiments, the viral envelope protein comprises the envelope protein from a different virus. In some embodiments, the viral envelope protein is the feline endogenous virus (RD114) envelope protein, an oncoretroviral amphotropic envelope protein, an oncoretroviral ecotropic envelope protein, the vesicular stomatitis virus envelope protein (VSV-G) (SEQ ID NO: 336), the baboon retroviral envelope glycoprotein (BaEV) (SEQ ID NO: 337), the murine leukemia envelope protein (MuLV) (SEQ ID NO: 338), the influenza glycoprotein HA surface glycoprotein (HA), the influenza glycoprotein neuraminidase (NA), the paramyxovirus Measles envelope protein H, the paramyxovirus Measles envelope protein F, the Tupaia paramyxovirus (TPMV) envelope protein H, the TPMV envelope protein F, glycoproteins G and F from the Henipavirus genus, the Nipah virus (NiV) envelope protein F, the NiV envelope protein G, the Sindbis virus (SINV) protein E1, the SINV protein E2, and/or functional variants or fragments of any of these envelope proteins (see, e.g., Frank and Bucholz Mol Ther Methods Clin Dev. 2018 Oct. 17; 12:19-31).

In some embodiments, the viral envelope protein can be wild-type BaEV. Not to be limited by theory, BaEV contains an R peptide that has been shown to inhibit transduction. In some embodiments, the BaEV can contain a deletion of the R peptide. In some embodiments, the BaEV can contain a deletion of the inhibitory R peptide after the nucleotides encoding the amino acid sequence HA, referred to herein as BaEVAR (HA) (SEQ ID NO: 339). In some embodiments, the BaEV can contain a deletion of the inhibitory R peptide after the nucleotides encoding the amino acid sequence HAM, referred to herein as BaEVAR (HAM) (SEQ ID NO: 340).

In some embodiments, the viral envelope protein can be wild-type MuLV. In some embodiments, the MuLV can contain one or more mutations to remove the furin-mediated cleavage site located between the transmembrane (TM) and surface (SU) subunits of the envelope glycoprotein. In some embodiments the MuLV contains the SUx mutation (MuLVSUx) (SEQ ID NO:372) which inhibits furin-mediated cleavage of MuLV envelope protein in packaging cells. In certain embodiments the C-terminus of the cytoplasmic tail of the MuLV or MuLVSUx protein is truncated by 4 to 31 amino acids. In certain embodiments the C-terminus of the cytoplasmic tail of the MuLV or MuLVSUx protein is truncated by 4, 8, 12, 16, 20, 24, 28, or 31 amino acids.

In some embodiments, the viral envelope protein include a binding polypeptide and a fusogenic polypeptide derived from different proteins. In one aspect, the viral envelope protein can comprise an influenza protein hemagglutinin HA and/or a neuraminidase (NA). In certain embodiments the HA is from influenza A virus subtype H1N1. In illustrative embodiments the HA is from H1N1 PR8 1934 in which the monobasic trypsin-dependent cleavage site has been mutated to a more promiscuous multibasic sequence (SEQ ID NO:311). In certain embodiments the NA is from influenza A virus subtype H10N7. In illustrative embodiments the NA is from H10N7-HKWF446C-07 (SEQ ID NO:312). In some embodiments, the binding polypeptide can be a functional variant or fragment of VSV-G, BaEV, BaEVAR (HA), BaEVAR (HAM), MuLV, MuLVSUx, influenza HA, influenza NA, or Measles envelope protein H that retains the ability to bind to a target cell, and the fusogenic polypeptide can be a functional variant or fragment of VSV-G, BaEV, BaEVAR (HA), BaEVAR (HAM), MuLV, MuLVSUx, influenza HA, influenza NA, or Measles envelope protein F that retains the ability to mediate fusion of the retroviral and target host cell membranes.

In another aspect, the viral envelope protein can include the fusion (F) and/or hemagglutinin (H) polypeptides of the measles virus (MV), as non-limiting examples, clinical wildtype strains of MV, and vaccine strains including the Edmonston strain (MV-Edm) (GenBank; AF266288.2) or fragments thereof. Not to be limited by theory, both hemagglutinin (H) and fusion (F) polypeptides are believed to play a role in entry into host cells wherein the H protein binds MV to receptors CD46, SLAM, and Nectin-4 on target cells and F mediates fusion of the retroviral and host cell membranes. In an illustrative embodiment, especially where the target cell is a T cell and/or NK cell, the binding polypeptide is a Measles Virus H polypeptide and the fusogenic polypeptide is a Measles Virus F polypeptide.

In some studies, lentiviral particles with viral envelope proteins including truncated F and H polypeptides had a significant increase in titers and transduction efficiency (Funke et al., 2008. Molecular Therapy. 16(8):1427-1436), (Frecha et al., 2008. Blood. 112(13):4843-4852). The highest titers were obtained when the F cytoplasmic tail was truncated by 30 residues (referred to as MV(Ed)-FΔ30 (SEQ ID NO:313)). For the H variants, optimal truncation occurred when 18 or 19 residues were deleted (MV(Ed)-HΔ18 (SEQ ID NO:314) or MV(Ed)-HΔ19), although variants with a truncation of 24 residues with and without replacement of deleted residues with alanine (MV(Ed)-HΔ24 (SEQ ID NO:315) and MV(Ed)-HΔ24+A) also resulted in optimal titers. Accordingly, in some embodiments, including those directed to transducing T cells and/or NK cells, the replication incompetent recombinant retroviral particles of the methods and compositions disclosed herein include mutated or variant versions of the measles virus fusion (F) and hemagglutinin (H) polypeptides, in illustrative examples, cytoplasmic domain deletion variants of measles virus F and H polypeptides. In some embodiments, the mutated F and H polypeptides are “truncated H” or “truncated F” polypeptides, whose cytoplasmic portion has been truncated, i.e., amino acid residues (or coding nucleic acids of the corresponding nucleic acid molecule encoding the protein) have been deleted. “HΔY” and “FΔX” designate such truncated H and F polypeptide, respectively, wherein “Y” refers to 1-34 residues that have been deleted from the amino termini and “X” refers to 1-35 residues that have been deleted from the carboxy termini of the cytoplasmic domains. In a further embodiment, the “truncated F polypeptide” is FΔ24 or FΔ30 and/or the “truncated H protein” is selected from the group consisting of HΔ14, HΔ15, HΔ16, HΔ17, HΔ18, HΔ19, HΔ20, HΔ21+A, HΔ24 and HΔ24+4A, more preferably HΔ18 or HΔ24. In an illustrative embodiment, the truncated F polypeptide is MV(Ed)-FΔ30 and the truncated H polypeptide is MV(Ed)-HΔ18.

In some embodiments, the viral envelope protein can be the envelope proteins from the Henipavirus genus (e.g., Nipah, Hendra, Cedar, Mojiang or Kumasi virus) and include envelope glycoprotein G (Henipavirus-G protein) and their fusion partner envelope glycoprotein F (Henipavirus-F protein). In some embodiments, the Henipavirus-F protein comprises the sequence of SEQ ID NO:374 and the Henipavirus-G protein comprises the sequence of SEQ ID NO:375. In some embodiments, the Henipavirus-F protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:374. In some embodiments, the Henipavirus-G protein comprises a sequence that has at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids of SEQ ID NO:375.

In some embodiments, the Henipavirus-G protein can contain one or more mutations to modify (e.g., truncate) the cytoplasmic tail and thus improve pseudotyping and particle incorporation efficiency (Palomares et al., 2013. J Virol. 87(8):4794-4794; Witting et al., 2013. Gene Ther. 20(10):997-1005; Bender et al., 2016. Plos Pathog. 12(6): e1005641). In certain embodiments, the N-terminus of the cytoplasmic tail of any of the Henipavirus-G proteins can be truncated by 1 to all of its amino acids. In some embodiments, the residues of the Henipavirus-G protein involved in receptor binding are mutated to alter, and in illustrative embodiments remove, their native interactions with their natural receptors. In certain embodiments, the Henipavirus-G protein is mutated for example, but not limited to, at one or more of Y389, E501, W504, E505, V507, Q530, E533, or 1588 of SEQ ID NO:375 (amino acids are given for Nipah-G, also referred to as NiV-G, and a skilled artisan will be able to identify the corresponding glutamines of other Henipavirus-G proteins)(Guillaume et al., 2006. J Virol. 80(15):7546-7554; Negrete et al., 2007. J Virol. 81(19):10804-10814; Xu et al., 2008. P Natl Acad Sci USA. 105(29):9953-9958; Xu et al., 2012. Plos One. 7(11): e48742; Bender et al., 2016. Plos Pathog. 12(6): e1005641). In some embodiments, Henipavirus-G protein is SEQ ID NO:375 with mutations E533A and/or Q530A. In some embodiments, one or more N- or O-glycosylation sites are mutated to improve pseudotyping and fusion (Biering et al., 2012. J Virol. 86(22):11991-12002; Stone et al., 2016. Plos Pathog. 12(2): e1005445). In some embodiments, one or more N-glycosylation sites are mutated for example, but not limited to, at one or more of N72, N159, N306, N378, N417, N481, or N529 of SEQ ID NO:375, or the corresponding glutamines of other Henipavirus-G proteins, to another amino acid such as glutamine. In some embodiments, one or more O-glycosylation sites are mutated from serine or threonine to another amino acid such as alanine. In some embodiments, one or more of the serine or threonine residues in the heavily 0-glycosylated stalk domain from amino acids 103 to 137 of SEQ ID NO:375, is mutated to, for example, alanine. In other embodiments, the C-terminus of the Henipavirus-G protein can be modified and fused to a binding polypeptide and in illustrative embodiments, an activation element, such as an antibody or antibody mimetic, which in illustrative embodiments can be an anti-CD3 antibody or antibody mimetic (Bender et al. 2016. Plos Pathog. 12(6): e1005641; Jamali et al. 2019. Mol Ther-Meth Clin D. 13:371-379; Frank et al. 2020. Blood Adv. 4(22):5702-5715).

In some embodiments, the F proteins can contain one or more mutations to modify (e.g., truncate) the cytoplasmic tail and thus improve pseudotyping, particle incorporation efficiency, and/or cleavage of the F protein from the inactive F0 to the cleaved active F1 form (Khetawat et al. 2010. Virol J. 7:312; Palomares et al. 2013. J Virol. 87(8):4794-4794; Witting et al. 2013. Gene Ther. 20(10):997-1005; Bender et al. 2016. Plos Pathog. 12(6): e1005641; Johnston et al. 2017. J Virol. 91(10): e02150-16). In some embodiments, one or more N-glycosylation sites are mutated for example, but not limited to, at one or more of N64, N67, N99, N414, or N 464 to another amino acid such as glutamine. In certain embodiments, the C-terminus of the cytoplasmic tail of the envelope glycoprotein F from the Henipavirus genus (Henipavirus-F protein) is truncated by 1 to all of its amino acids. In some embodiments, the F protein can contain one or more mutations to make it more fusogenic (Aguilar et al. 2007. J Virol. 81(9):4520-4532; Weis et al. 2015. Eur J Cell Biol. 94(7-9):316-322).

In some embodiments, the viral envelope protein can include a Henipavirus-F protein and a Henipavirus-G protein from the same virus of the Henipavirus genus (i.e., homologous proteins). In some embodiments, the viral envelope protein can include a Henipavirus-F protein and a Henipavirus-G protein from different viruses of the Henipavirus genus (i.e., heterologous proteins). In some embodiments, the viral envelope protein can include a Henipavirus-F protein and a Henipavirus-G protein can be chimeras composed of domains of heterologous proteins (Bradel-Tretheway et al. 2019. J Virol. 93(13): e00577-19).

In some embodiments, any of the viral envelope protein can contain one or more mutations to modify (e.g., truncate) the cytoplasmic tail and thus improve pseudotyping, and particle incorporation efficiency. In certain embodiments, the N-terminus of the cytoplasmic tail is truncated by 1 to all of its amino acids. In some embodiments, the residues involved in receptor binding are mutated to alter, and in illustrative embodiments remove, their native interactions with their natural receptors. Similar to the mutations for Nipah-G protein, in some embodiments, the VSV-G protein is mutated for example, but not limited to, in the residues K47 or R354, for example K47A or K47Q and/or R354A or R354Q. In some embodiments, these viral envelope protein are fused to heterologous binding polypeptides that function to direct or redirect the viral envelope protein to a new target protein on the same or different cell target.

In some embodiments, the viral envelope protein (e.g., pseudotyping element and/or the fusogenic membrane polypeptide) is derived from envelope glycoproteins of a virus of the Paramyxoviridae family. The virus of the Paramyxoviridae family is preferably a virus of the Morbillivirus genus or of the Henipavirus genus. The viruses of the Morbillivirus genus and of the Henipavirus genus use two types of glycoproteins to enter into a target cell: an attachment protein (called glycoprotein G in a virus of the Henipavirus genus or glycoprotein H in a virus of the Morbillivirus genus) and a glycoprotein F (also called fusion protein or protein F). The protein F mediates the fusion of viral membranes with the cellular membranes of the host cell. The glycoprotein G/H recognizes the receptor on the target membrane and supports the F protein in its membrane fusion function. Both glycoprotein G/H and glycoprotein F are used in a modified form for pseudotyping the retrovirus-like particle or retroviral vector according to the invention. A virus of the Morbillivirus genus is for example selected in the group consisting of measles virus, Canine distemper virus, Cetacean morbillivirus, Peste-des-petits-ruminants virus, Phocine distemper virus and Rinderpest virus. A preferred virus of the Morbillivirus genus is a measles virus (MeV). A virus of the Henipavirus genus is for example selected in the group consisting of Nipah virus, Cedar virus and Hendra virus. A preferred virus of the Henipavirus genus is a Nipah virus (NiV). In a preferred embodiment, the modified enveloped glycoproteins are derived from the envelope glycoprotein H and the glycoprotein F of a measles virus or from the envelope glycoprotein G and the glycoprotein F of a Nipah virus. An example of sequence of Nipah virus envelope glycoprotein G is sequence SEQ ID NO: 9 of WO 2017/182585. An example of sequence of Nipah virus envelope glycoprotein F is sequence SEQ ID NO: 11 of WO 2017/182585. An example of sequence of measles virus envelope glycoprotein H (called glycoprotein H) is sequence SEQ ID NO: 10 of WO 2017/182585. An example of sequence of measles virus envelope glycoprotein F is sequence SEQ ID NO: 12 of WO 2017/182585. WO 2017/182585 is incorporated herein by reference in its entirety.

In some embodiments, the separate binding and/or fusogenic polypeptides comprise one or more non virally-derived proteins. In some embodiments the binding polypeptide comprises an antibody, a ligand, or a receptor that binds a polypeptide on the target cell. In some embodiments, the binding polypeptide comprises an alternative non-antibody scaffold, also referred to herein as an antibody mimetic. In any of the aspects or embodiments provided herein that include a binding polypeptide, the binding polypeptide can be an antibody mimetic. In any of the aspects or embodiments provided herein that include a binding polypeptide that is an antibody, a suitable antibody mimetic can be used instead of the antibody. In some embodiments, the antibody mimetic can be an affibody, an afflilin, an affimer, an affitin, an alphabody, an alphamab, an anticalin, a peptide aptamer, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a nanofitin, a leucine-rich repeat domain, a lipocalin domain, a mAb 2 or Fcab™, a nanobody, a nanoCLAMP, an OBody, a Pronectin, a single-chain TCR, a tetratricopeptide repeat domain, a VHH, or a V-like domain. In some embodiments the binding polypeptide recognizes a protein on the surface of NK cells such as CD16, CD56, and CD57. In some embodiments the binding polypeptide recognizes a protein on the surface of T cells such as CD3, CD4, CD8, CD25, CD28, CD62L, CCR7, TCRa, and TCRb. In some embodiments, the binding polypeptide is also the activation element. In some embodiments, the binding polypeptide is a membrane polypeptide that binds CD3. In some embodiments, the fusogen is derived from the Sindbis virus glycoprotein that is modified to remove its binding activity, SV1, and the binding polypeptide is a membrane-bound anti-CD3 antibody (Yang et al. 2009. Pharm Res 26(6):1432-1445).

In some embodiments, the viral particles include viral envelope proteins from 2 or more heterologous viruses (e.g., are co-pseudotyped). In some embodiments, the viral particles include VSV-G, or a functional variant or fragment thereof, and an envelope protein from RD114, BaEV, MuLV, influenza virus, measles virus, and/or a functional variant or fragment thereof. In some embodiments, the viral particles include VSV-G and the MV(Ed)-H glycoprotein or the MV(Ed)-H glycoprotein with a truncated cytoplasmic domain. In illustrative embodiments, the viral particles include VSV-G and MV(Ed)-HΔ24. In certain embodiments, the viral particles include VSV-G with MuLV or MuLV with a truncated cytoplasmic domain. In other embodiments, the viral particles include VSV-G MuLVSUx or MuLVSUx with a truncated cytoplasmic domain. In further illustrative embodiments, the viral particles include VSV-G with a fusion of an antiCD3scFv to MuLV.

In some embodiments, the viral envelope proteins are altered to reduce their endogenous binding activity to its natural target. In some embodiments, VSV-G is altered to reduce its endogenous binding activity to LDL-R. In some embodiments, the viral envelope protein comprises a VSV-G variant that retains at least 75%, 80%, 85%, 90%, 95%, 96%, 97%, 98%, or 99% identity with the amino acid sequence of SEQ ID NO:336 and has one, two, three, or more mutations at a position corresponding to amino acids residues 8, 47, 209, and 354.

In some embodiments, the fusogenic polypeptide is derived from a class I fusogen. In some embodiments, the fusogenic polypeptide is derived from a class II fusogen. In some embodiments, both the binding polypeptide and the separate fusogenic polypeptide are virally-derived. In some embodiments, the fusogenic polypeptide includes multiple elements expressed as one polypeptide. In some embodiments, the binding polypeptide and fusogenic polypeptide are translated from the same transcript but from separate ribosome binding sites; in other embodiments, the binding polypeptide and fusogenic polypeptide are separated by a cleavage peptide site, which not to be bound by theory, is cleaved after translation, as is common in the literature, or a ribosomal skip sequence. In some embodiments, the translation of the binding polypeptide and fusogenic polypeptide from separate ribosome binding sites results in a higher amount of the fusogenic polypeptide as compared to the binding polypeptide. In some embodiments, the ratio of the fusogenic polypeptide to the binding polypeptide is at least 2:1, at least 3:1, at least 4:1, at least 5:1, at least 6:1, at least 7:1, or at least 8:1. In some embodiments, the ratio of the fusogenic polypeptide to the binding polypeptide is between 1.5:1, 2:1, or 3:1, on the low end of the range, and 3:1, 4:1, 5:1, 6:1, 7:1, 8:1, 9:1 or 10:1 on the high end of the range.

In embodiments disclosed herein including short contacting times, many of the modified lymphocytes in a cell formulation have viral envelope proteins on their surfaces during reintroduction of the modified lymphocytes into the subject, either through association with the replication incompetent recombinant retroviral particle or by fusion of the retroviral envelopes with the plasma membranes of the modified lymphocytes. In some embodiments, at least 10%, 15%, 20%, 25%, 30%, 35%, 40%, 45%, 50%, 55%, 60%, 65%, 70%, 75%, 80%, 85%, or 90% of the modified lymphocytes in the cell formulation can include a viral envelope protein on their surfaces. In some embodiments, the viral envelope protein can be bound to the surface of the modified lymphocytes and/or the viral envelope protein can be present in the plasma membrane of the modified lymphocytes.

In some embodiments, RIPs herein include on their surface, a means for binding to a T cell and/or NK cell. In some embodiments, RIPs herein include on their surface, a means for mediating fusion of the RIP and T cell and/or NK cell membranes. In some embodiments, RIPs herein include on their surface, both a means for binding to a T cell and/or NK cell, and a means for mediating fusion of the RIP and T cell and/or NK cell membranes. In some embodiments, this is the same means.

In some embodiments, any of the RIP (RIP formulation and/or a delivery solution) for direct administrating to the subject as disclosed herein can include envelope proteins on their surface, for example, multiple copies of a T cell and/or NK cell binding polypeptide and multiple copies of a fusogenic polypeptide, also called a fusogen as disclosed herein. In some embodiments, any of the RIP (RIP formulation and/or a delivery solution) for direct administrating to the subject as disclosed herein can include on their surface, a means for binding to a T cell and/or NK cell as disclosed herein.

### Activation Elements

Many of the methods and composition aspects of the present disclosure that include a replication incompetent recombinant retroviral particle further include an activation element, also referred to herein as a T cell activation element, or a nucleic acid encoding an activation element. Activation elements are envelope proteins of the replication incompetent recombinant retroviral particles. Cells of the immune system such as T lymphocytes recognize and interact with specific antigens through receptors or receptor complexes which, upon recognition or an interaction with such antigens, cause activation of the cell and expansion in the body. An example of such a receptor is the antigen-specific T lymphocyte receptor complex (TCR/CD3) expressed on the surface of T lymphocytes. The TCR recognizes antigenic peptides that are presented to it by the proteins of the major histocompatibility complex (MHC) on the surface of antigen presenting cells and other T lymphocyte targets. Stimulation of the TCR/CD3 complex results in activation of the T lymphocyte and a consequent antigen-specific immune response. Thus, activation elements provided herein, activate T cells by binding to one or more components of the T cell receptor associated complex, for example by binding to CD3. In some embodiments, the activation element can activate alone. In other cases, the activation requires activation through the TCR receptor complex in order to further activate cells. T lymphocytes also require a second, co-stimulatory signal to become fully active in vivo. Without such a signal, T lymphocytes are either non-responsive to antigen binding to the TCR, or become anergic. However, the second, co-stimulatory signal is not required for the transduction and expansion of T cells and can be provided, for example, by a later co-stimulatory signal from a CAR or LE after transduction, as provided elsewhere herein. In some embodiments, the co-stimulatory signal can be provided during transduction by, for example, CD28, a T lymphocyte protein, which interacts with CD80 and CD86 on antigen-producing cells.

Activation of the T cell receptor (TCR) CD3 complex and co-stimulation with CD28 can occur by ex vivo exposure to solid surfaces (e.g., beads) coated with anti-CD3 and anti-CD28. In some embodiments of the methods and compositions disclosed herein, resting T cells are activated by exposure to solid surfaces coated with anti-CD3 and anti-CD28 ex vivo. In other embodiments, resting T cells or NK cells, and in illustrative embodiments resting T cells, are activated by exposure to soluble anti-CD3 antibodies (e.g., at 50-150, or 75-125, or 100 ng/ml). In such embodiments, which can be part of methods for modifying, genetically modifying or transducing, in illustrative embodiments without prior activation, such activation and/or contacting can be carried out by including anti-CD3 in a transduction reaction mixture and contacting with optional incubating for any of the times provided herein. Furthermore, such activation with soluble anti-CD3 can occur by incubating lymphocytes, such as PBMCs, and in illustrative embodiments NK cells and in more illustrative embodiments, T cells, after they are contacted with retroviral particles in a media containing an anti-CD3. Such incubation can be for example, for between 5, 10, 15, 30, 45, 60, or 120 minutes on the low end of the range, and 15, 30, 45, 60, 120, 180, or 240 minutes on the high end of the range, for example, between 15 and 1 hours or 2 hours.

In certain illustrative embodiments of the methods, kits, and compositions provided herein, for example for modifying (in vivo or ex vivo), genetically modifying, and/or transducing lymphocytes, especially T cells and/or NK cells, polypeptides that are capable of binding to an activating T cell surface protein are presented as “activation elements” on the surface of replication incompetent recombinant retroviral particles. Thus, in some embodiments, an activation element can perform the binding polypeptide function. In some embodiments, the activation element is an envelope protein. Such T cell and/or NK cell activation elements on the surface of a retroviral particle are present in embodiments herein for modifying, genetically modifying, and/or transducing lymphocytes, for example wherein the retroviral particle has a genome that encodes a CAR, TCR, or LE. In some embodiments, any of the RIP formulations or delivery formulations comprising RIPs as disclosed herein for direct administration to a subject can comprise activation elements on the surface of RIPs as disclosed herein. In some embodiments, such retroviral particles whose surface has an activation element are used in methods and uses that include administration by subcutaneous administration, and in kit components for subcutaneous administration. The activation element function discussed herein this section, as well as the binding polypeptide and fusogenic polypeptide disclosed elsewhere herein, in certain illustrative embodiments are all found associated with the surface of a retroviral particle, as part of one, two, or three proteins, in illustrative embodiments glycoproteins, and in further illustrative embodiments, heterologous glycoproteins. For example, some activation element polypeptides, such as those that are capable of binding to CD3, can also provide a T cell binding polypeptide function.

In some embodiments, the activation element is a polypeptide capable of binding to a polypeptide on the surface of a lymphocyte, and in illustrative embodiments, a T cell and/or an NK cell. In illustrative embodiments, the activation element is capable of binding to a TCR complex polypeptide. In some embodiments, a TCR complex polypeptide is CD3D, CD3E, CD3G, CD3Z, TCRα, or TCRβ. In some embodiments, the activation element capable of binding to the TCR complex polypeptide is a polypeptide capable of binding to one or more of CD3D, CD3E, CD3G, CD3Z, TCRα, or TCRβ. In illustrative embodiments, the activation element activates ZAP-70. In some embodiments, the activation element includes a polypeptide capable of binding to CD16A, NKG2C, NKG2D, NKG2E, NKG2F, or NKG2H. In some embodiments, the polypeptide capable of binding to NKG2D is MIC-A, MIC-B, or a ULBP, for example ULBP1 or ULBP2. In further embodiments, the polypeptide capable of binding to CD16A includes capable of binding to one or more of NKp46, 2B4, CD2, DNAM, NKG2C, NKG2D, NKG2E, NKG2F, or NKG2H. In some embodiments, the activation element is a polypeptide capable of binding to one or more of the following combinations: NKp46 and 2B4, NKp46 and CD2, NKp46 and DNAM, NKp46 and NKG2D, 2B4 and DNAM, or 2B4 and NKG2D. In some embodiments, the activation element can be two or more polypeptides capable of binding to polypeptides on the surface of a lymphocyte. In some embodiments, the activation element can be one or more polypeptides capable of binding to at least one of the following combinations: NKp46 and 2B4, NKp46 and CD2, NKp46 and DNAM, NKp46 and NKG2D, 2B4 and DNAM, or 2B4 and NKG2D. In illustrative embodiments the activation element is a polypeptide capable of binding to CD3E. In some embodiments, the polypeptide capable of binding to CD3 is an anti-CD3 antibody, or a fragment thereof that retains the ability to bind to CD3. In illustrative embodiments, the anti-CD3 antibody or fragment thereof is a single chain anti-CD3 antibody, such as but not limited to, an anti-CD3 scFv. In another illustrative embodiment, the polypeptide capable of binding to CD3 is anti-CD3scFvFc.

In some embodiments, the activation element is an antibody. In some embodiments, the activation element comprises an alternative non-antibody scaffold, also referred to herein as an antibody mimetic. In any of the aspects or embodiments provided herein that include an activation element capable of binding to a polypeptide on the surface of a lymphocyte, and in illustrative embodiments, a T cell, the binding polypeptide can be an antibody mimetic. In some embodiments, the antibody mimetic can be an affibody, an afflilin, an affimer, an affitin, an alphabody, an alphamab, an anticalin, a VHH, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain. In any of the aspects or embodiments provided herein that include an activation element that is an antibody, a suitable antibody mimetic can be used instead of the antibody. In some embodiments, the activation element capable of binding a polypeptide on the surface of a lymphocyte (e.g., TCRβ) is a superantigen polypeptide.

RIPs provided herein in some embodiments, can include on their surface, a means for binding CD3. A number of anti-human CD3 monoclonal antibodies and antibody fragments thereof are available, and can be used in the present invention, including but not limited to UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XIII46, XIII-87, 12F6, T3/RW2-8C8, T3/RW24B6, OKT3D, M-T301, SMC2 and F101.01. In illustrative embodiments, the anti-CD3 is UCHT1 or OKT-3. In some embodiments, the anti-CD3 comprises the VHH antibody CML3.1 (SEQ ID NO: 421). In some embodiments, the anti-CD3 comprises the sequence of UCHT1 in SEQ ID NO: 347.

In other embodiments, the activation element on the surfaces of the replication incompetent recombinant retroviral particles can include one or more polypeptides capable of binding CD2, CD28, OX40, 4-1BB, ICOS, CD9, CD53, CD63, CD81, and/or CD82 and optionally one or more polypeptides capable of binding CD3. In illustrative embodiments, the activation element is a polypeptide capable of binding a mitogenic tetraspanin, for example, a polypeptide capable of binding to CD81, CD9, CD53, CD63, or CD82. In some embodiments, the activation element is a tetraspanin. Tetraspanins are known in the art. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-1/A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UPIb, UPK1B), TSPAN21 (UPla, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), or TSPAN33. In some embodiments, the tetraspanin can be TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231/TALLA-1/A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UPIb, UPK1B), TSPAN21 (UPIa, UPK1A), TSPAN22 (RDS, PRPH2), TSPAN23 (ROM1), TSPAN24 (CD151), TSPAN26 (CD37), TSPAN31 (SAS), TSPAN32 (TSSC6), or TSPAN33. In illustrative embodiments, the tetraspanin is TSPAN7 (CD231/TALLA-1/A15), TSPAN9 (NET-5), TSPAN24 (CD151), TSPAN27 (CD82), TSPAN28 (CD81), TSPAN29 (CD9), or TSPAN30 (CD63). In some embodiments, the activation element is a tetraspanin, and the tetraspanin is TSPAN25 (CD53), TSPAN27 (CD82), TSPAN28 (CD81), TSPAN29 (CD9), or TSPAN30 (CD63). In some embodiments, a tetraspanin is the only envelope protein. In some embodiments, a tetraspanin is a pseudotyping element comprising the binding polypeptide and the fusogenic element. In some embodiments, a tetraspanin is the activation element and the pseudotyping element. In illustrative embodiments, the tetraspanin that is the activation element and the pseudotyping element is TSPAN29 (CD9).

In some embodiments, one or typically more copies of one or more of these activation elements can be expressed on the surfaces of the replication incompetent recombinant retroviral particles as polypeptides separate and distinct from the pseudotyping elements. In some embodiments, the activation elements can be expressed on the surfaces of the replication incompetent recombinant retroviral particles as fusion polypeptides. In illustrative embodiments, the fusion polypeptides include one or more activation elements and one or more pseudotyping elements or one or more binding and/or fusogenic elements. In further illustrative embodiments, the fusion polypeptide includes anti-CD3, for example an anti-CD3scFv, or an anti-CD3scFvFc, and a viral envelope protein. In one example the fusion polypeptide is the OKT-3scFv fused to the amino terminal end of a viral envelope protein such as the MuLV envelope protein, as shown in Maurice et al. (2002). In some embodiments, the fusion polypeptide is UCHT1scFv fused to a viral envelope protein, for example the MuLV envelop protein (SEQ ID NO:341), the MuLVSUx envelope protein (SEQ ID NO:366), VSV-G (SEQ ID NO:367), or functional variants or fragments thereof, including any of the membrane protein truncations provided herein. In illustrative embodiments, especially for compositions and methods herein for transducing lymphocytes in whole blood, the fusion polypeptide does not include any blood protein (e.g., blood Factor (e.g., Factor X)) cleavage sites in the portion of the fusion protein that resides outside the retroviral particle. In some embodiments, the fusion constructs do not include any furin cleavage sites. Furin is a membrane bound protease expressed in all mammalian cells examined, some of which is secreted and active in blood plasma (See e.g., C. Fernandez et al. J. Internal. Medicine (2018) 284; 377-387). Mutations can be made to fusion constructs using known methods to remove such protease cleavage sites.

Polypeptides that bind CD3, CD28, OX40, 4-1BB, or ICOS are referred to as activation elements because of their ability to activate resting T cells. In certain embodiments, nucleic acids encoding such an activation element are found in the genome of a replication incompetent recombinant retroviral particle that contains the activating element on its surface. In illustrative embodiments, nucleic acids encoding an activation element are not found in the replication incompetent recombinant retroviral particle genome. In some embodiments, the nucleic acids encoding an activation element are found in the genome of a virus packaging cell.

In some embodiments, the activation element is a polypeptide capable of binding to CD28, for example, an anti-CD28 antibody or an anti-CD28 scFv antibody, or a fragment thereof that retains the ability to bind to CD28. In other embodiments, the polypeptide capable of binding to CD28 is CD80, CD86, or a functional fragment thereof that is capable of binding CD28 and inducing CD28-mediated activation of Akt, such as an external fragment of CD80. In some aspects herein, an external fragment of CD80 means a fragment that is typically present on the outside of a cell in the normal cellular location of CD80, that retains the ability to bind to CD28.

Anti-CD28 antibodies are known in the art and can include, as non-limiting examples, the monoclonal antibodies 9.3 (an IgG2a antibody), KOLT-2 (an IgG1 antibody), 15E8 (an IgG1 antibody), 248.23.2 (an IgM antibody), and EX5.3D10 (an IgG2a antibody).

In an illustrative embodiment, an activation element includes two polypeptides, a polypeptide capable of binding to CD3 and a polypeptide capable of binding to CD28.

In certain embodiments, the polypeptide capable of binding to CD3 or CD28 is an antibody, a single chain monoclonal antibody or an antibody fragment, for example a single chain antibody fragment. Accordingly, the antibody fragment can be, for example, a single chain fragment variable region (scFv), an antibody binding (Fab) fragment of an antibody, a single chain antigen-binding fragment (scFab), a single chain antigen-binding fragment without cysteines (scFabAC), a fragment variable region (Fv), a construct specific to adjacent epitopes of an antigen (CRAb), or a single domain antibody (VH or VL).

In some embodiments, the activation element can include a bispecific T-cell engager (BiTE), as known in the art, which binds to two separate cell surface proteins. In some embodiments, a BiTE can include one or more polypeptides capable of binding to an activating T cell surface protein and to another surface protein. In some embodiments, a BiTE can include one or more polypeptides capable of binding to an activating T cell surface protein and to a surface protein another cell, for example, a blood cell, and in illustrative embodiments, a B cell. In some embodiments, a BiTE can include one or more polypeptides capable of binding to CD3. In some embodiments, a polypeptide capable of binding CD3 can be any of the polypeptides herein that bind CD3, for example, an anti-CD3 antibody. In some embodiments, a BiTE can include one or more polypeptides capable of binding to CD19. In some embodiments, a polypeptide capable of binding to CD19 can be an anti-CD19 antibody, including any anti-CD19 antibody known in the art. In illustrative embodiments, the BiTe can include one or more polypeptides capable of binding to CD3 and CD19.

In some embodiments, at least 10%, 15%, 20%, 25%, 30%, 35%, 40%, 45%, 50%, 55%, 60%, 65%, 70%, 75%, 80%, 85%, or 90% of the modified lymphocytes in a cell formulation can include a T cell activation element on their surfaces. In some embodiments, the T cell activation element can be bound to the surface of the modified lymphocytes through, for example, a T cell receptor, and/or the pseudotyping element can be present in the plasma membrane of the modified lymphocytes.

In any of the embodiments disclosed herein, an activation element, or a nucleic acid encoding the same, can include a dimerizing or higher order multimerizing motif Dimerizing and multimerizing motifs are well-known in the art and a skilled artisan will understand how to incorporate them into the polypeptides for effective dimerization or multimerization. In illustrative embodiments, the polypeptide capable of binding to CD3 is anti-CD3scFvFc, which in some embodiments is considered an anti-CD3 with a dimerizing motif without any additional dimerizing motif, since anti-CD3scFvFc constructs are known to be capable of dimerizing without the need for a separate dimerizing motif.

In some embodiments, when present on the surface of replication incompetent recombinant retroviral particles, an activation element including a dimerizing motif can be active in the absence of a dimerizing agent. In some embodiments, the dimerizing or multimerizing motif, or a nucleic acid sequence encoding the same, can be an amino acid sequence from transmembrane polypeptides that naturally exist as homodimers or multimers. In some embodiments, the dimerizing or multimerizing motif, or a nucleic acid sequence encoding the same, can be an amino acid sequence from a fragment of a natural protein or an engineered protein. In one embodiment, the homodimeric polypeptide is a leucine zipper motif-containing polypeptide (leucine zipper polypeptide). For example, a leucine zipper polypeptide derived from c-JUN, non-limiting examples of which are disclosed related to chimeric lymphoproliferative elements (CLEs) herein. In some embodiments, these transmembrane homodimeric polypeptides can include CD69, CD71, CD72, CD96, Cd105, Cd161, Cd162, Cd249, CD271, Cd324, or active fragments thereof.

In some embodiments, when present on the surface of replication incompetent recombinant retroviral particles, an activation element including a dimerizing motif can be active in the presence of a dimerizing agent. In some embodiments, the dimerizing motif, and nucleic acid encoding the same, can include an amino acid sequence from transmembrane proteins that dimerize upon ligand (also referred to herein as a dimerizer or dimerizing agent) binding. In some embodiments, the dimerizing motif and dimerizer can include (where the dimerizer is in parentheses following the dimerizer-binding pair): FKBP and FKBP (rapamycin or its analog AP1903); FKBP12-F36V and FKBP12-F36V (AP1903); FKBP and FRB (rapamycin or its analog); GyrB and GyrB (coumermycin or its analog); DHFR and DHFR (methotrexate); or DmrB and DmrB (AP20187). As noted above, rapamycin can serve as a dimerizer. Alternatively, a rapamycin derivative or analog can be used (see, e.g., WO96/41865; WO 99/36553; WO 01/14387; and Ye et al (1999) Science 283:88-91). A coumermycin analog can be used (see, e.g., Farrar et al. (1996) Nature 383:178-181; and U.S. Pat. No. 6,916,846). Although some embodiments of lymphoproliferative elements include a dimerizing agent, in some aspects and illustrative embodiments, a lymphoproliferative element is constitutively active, and is other than a lymphoproliferative element that requires a dimerizing agent for activation.

In some embodiments, an activation element is fused to a heterologous signal sequence and/or a heterologous membrane attachment sequence or a membrane bound protein, all of which help direct the activation element to the membrane. In some embodiments, posttranslational lipid modification can occur via myristoylation, palmitoylation, or GPI anchorage. In some embodiments, the heterologous membrane attachment sequence is a GPI anchor attachment sequence. The heterologous GPI anchor attachment sequence can be derived from any known GPI-anchored protein. In some embodiments, the heterologous GPI anchor attachment sequence is the GPI anchor attachment sequence from CD14, CD16, CD48, CD55 (DAF), CD59, CD80, and CD87. In some embodiments, the heterologous GPI anchor attachment sequence is derived from CD16. In illustrative embodiments, the heterologous GPI anchor attachment sequence is derived from Fc receptor FcγRIIIb (CD16b) or decay accelerating factor (DAF), otherwise known as complement decay-accelerating factor or CD55.

In some embodiments, one or more of the activation elements include a heterologous signal sequence to help direct expression of the activation element to the cell membrane. Any signal sequence that is active in the packaging cell line can be used. In some embodiments, the signal sequence is a DAF signal sequence. In illustrative embodiments, an activation element is fused to a DAF signal sequence at its N terminus and a GPI anchor attachment sequence at its C terminus.

In an illustrative embodiment, the activation element includes anti-CD3 scFvFc fused to a GPI anchor attachment sequence derived from CD14 and CD80 fused to a GPI anchor attachment sequence derived from CD16b; and both are expressed on the surface of a replication incompetent recombinant retroviral particle provided herein. In some embodiments, the anti-CD3 scFvFc is fused to a DAF signal sequence at its N terminus and a GPI anchor attachment sequence derived from CD14 at its C terminus and the CD80 is fused to a DAF signal sequence at its N terminus and a GPI anchor attachment sequence derived from CD16b at its C terminus; and both are expressed on the surface of a replication incompetent recombinant retroviral particle provided herein. In some embodiments, the DAF signal sequence includes amino acid residues 1-30 of the DAF protein.

In some embodiments, an activation element can be separate from the replication incompetent recombinant retroviral particle. Thus, in some embodiments, the replication incompetent recombinant retroviral particles do not comprise an activation element on their surface.

In some embodiments, more than one activation element is used. In some embodiments, the activation element can be a superantigen, for example lipopolysaccharide, SEC3, and Staphylococcal enterotoxin B. In some embodiments, the activation element can be a cytokine. In some embodiments, the activation element can be phorbol myristate acetate (PMA), ionomycin, or phytohemagglutinin (PHA). In some embodiments, the concentration of PMA in a cell formulation or to be administered separately from the replication incompetent recombinant retroviral particles can be 10, 25, 50, 75, or 100 ng/ml or between 10 and 100 ng/ml or 25 and 75 ng/ml. In some embodiments, the concentration of ionomycin in a cell formulation or to be administered separately from the replication incompetent recombinant retroviral particles can be at least or about 100, 250, 500, or 750 ng/ml or 1, 2, 3, 4, or 5 μg/ml or between 100 ng/ml and 5 μg/ml or between 500 ng/ml and 2 μg/ml. In some embodiments, the concentration of PHA in a cell formulation or to be administered separately from the replication incompetent recombinant retroviral particles can be at least or about 0.1 μg/ml, 0.25 μg/ml, 0.5 μg/ml, 1 μg/ml, 2.5 μg/ml, 5 μg/ml, 7.5 μg/ml, or 10 μg/ml or between 0.1 and 10 μg/ml, 1 and 10 μg/ml, or 2.5 and 7.5 μg/ml. In some embodiments, the activation element is administered within 5, 10, 15, 20, 30, 45, or 60 minutes, or 1, 2, 3, 4, 5, 6, 7, 8, 10, 12, 18, or 24 hours or 1, 2, 3, 4, 5, 6, 7, 14, 21, or 28 days of administering a cell formulation. In some embodiments, the activation element or elements are administered multiple times, for example on different days following administration of the cell formulation.

### Retroviral Genome Size

In the methods and compositions provided herein that include RIPs, genomes of the RIPs (i.e., the recombinant retroviral genomes), in non-limiting illustrative examples, lentiviral genomes, have a limitation to the number of polynucleotides that can be packaged into the viral particle. As disclosed elsewhere herein, references to retroviral particles or RIPs herein can In some embodiments provided herein, the polypeptides encoded by the polynucleotide encoding region can be truncations or other deletions that retain a functional activity such that the polynucleotide encoding region is encoded by less nucleotides than the polynucleotide encoding region for the wild-type polypeptide. In some embodiments, the polypeptides encoded by the polynucleotide encoding region can be fusion polypeptides that can be expressed from one promoter. In some embodiments, the fusion polypeptide can have a cleavage signal to generate two or more functional polypeptides from one fusion polypeptide and one promoter. Furthermore, some functions that are not required after initial ex vivo transduction are not included in the retroviral genome, but rather are present on the surface of the replication incompetent recombinant retroviral particles via the packaging cell membrane. These various strategies are used herein to maximize the functional elements that are packaged within the replication incompetent recombinant retroviral particles.

In some embodiments, the recombinant retroviral genome to be packaged can be between 1,000, 2,000, 3,000, 4,000, 5,000, 6,000, 7,000, and 8,000 nucleotides on the low end of the range and 2,000, 3,000, 4,000, 5,000, 6,000, 7,000, 8,000, 9,000, 10,000, and 11,000 nucleotides on the high end of the range. The retroviral genome to be packaged includes one or more polynucleotide regions encoding a first and second engineering signaling polypeptide as disclosed in detail herein. In some embodiments, the recombinant retroviral genome to be packaged can be less than 5,000, 6,000, 7,000, 8,000, 9,000, 10,000, or 11,000 nucleotides. Functions discussed elsewhere herein that can be packaged include required retroviral sequences for retroviral assembly and packaging, such as a retroviral rev, gag, and pol coding regions, as well as a 5′ LTR and a 3′ LTR, or an active truncated fragment thereof, a nucleic acid sequence encoding a retroviral cis-acting RNA packaging element, and a cPPT/CTS element.

Furthermore, in illustrative embodiments a replication incompetent recombinant retroviral particle herein can include any one or more or all of the following, in some embodiments in reverse orientation with respect to a 5′ to 3′ orientation established by the retroviral 5′ LTR and 3′ LTR (as illustrated in WO2019/055946 as a non-limiting example): one or more polynucleotide regions encoding a first and second engineering signaling polypeptide, at least one of which includes at least one lymphoproliferative element (e.g., that each comprises 1 or 2 lymphoproliferative element polypeptides); a second engineered signaling polypeptide that can include a chimeric antigen receptor; an miRNA, a control element, such as a riboswitch, which typically regulates expression of the first and/or the second engineering signaling polypeptide; a safety switch polypeptide, an intron, a promoter that is active in a target cell, such as a T cell, a 2A cleavage signal and/or an IRES.

### Kits and Commercial Products

In another aspect, provided herein is a delivery composition or suspension, for example for treating or preventing a disease, for example cancer or tumor growth, comprising polynucleotides, such as polynucleotide vectors, in illustrative replication incompetent recombinant retroviral particle (RIPs), or modified cells, such as modified lymphocytes, modified TILs, modified lymphocytes other than B cells, or modified T cells and/or modified NK cells as an active ingredient. In another aspect, provided herein is an infusion composition or other RIP or cell formulation for treating or preventing cancer or tumor growth comprising polynucleotides such as polynucleotide vectors, in illustrative embodiments RIPs, or modified cells, such as modified lymphocytes, modified TILs, modified lymphocytes other than B cells, or modified T cells and/or modified NK cells. The polynucleotides such as polynucleotide vectors, in illustrative embodiments RIPs, or modified cells, such as modified lymphocytes, modified TILs, modified lymphocytes other than B cells, or modified T cells and/or modified NK cells of the delivery composition or infusion composition can include any of the aspects, embodiments, or subembodiments discussed above or elsewhere herein, for example that include nucleic acids that encode anti-idiotype polypeptides, a CAR, an LE, a cytokine and/or a TCR.

Provided herein in one aspect is a container, such as a commercial container or package, or a kit comprising the same, comprising polynucleotides, such as polynucleotide vectors, for example RIPs, or modified cells, such as modified lymphocytes, modified TILs, modified lymphocytes other than B cells, or modified T cells and/or modified NK cells according to any of the aspects and embodiments provided herein. As a non-limiting example, the polynucleotides, such as polynucleotide vectors, for example RIPs, or modified cells, such as modified lymphocytes, modified TILs, modified lymphocytes other than B cells, or modified T cells and/or modified NK cells can comprise in their genome a polynucleotide comprising one or more nucleic acid sequences operatively linked to a promoter active in T cells and/or NK cells. In some embodiments, a nucleic acid sequence of the one or more nucleic acid sequences can encode an anti-idiotype polypeptide, an inhibitory RNA, a cytokine, a lymphoproliferative element, a TCR, and/or a chimeric antigen receptor (CAR) comprising an antigen-specific targeting region (ASTR), a transmembrane domain, and an intracellular activating domain. In some embodiments, a nucleic acid sequence of the one or more nucleic acid sequences can encode one, two or more inhibitory RNA molecules directed against one or more RNA targets.

The container that contains the polynucleotides, such as polynucleotide vectors, for example RIPs, or modified cells, such as modified lymphocytes, modified TILs, modified lymphocytes other than B cells, or modified T cells and/or modified NK cells in any aspect or embodiment including a commercial container as well as kits, can be an infusion bag, tube, vial, well of a plate, or other vessel for storage of polynucleotides, such as polynucleotide vectors, for example RIPs, or modified cells, such as modified lymphocytes, modified TILs, modified lymphocytes other than B cells, or modified T cells and/or modified NK cells. In fact, some aspects provided herein, comprise a container comprising polynucleotides, such as polynucleotide vectors, for example RIPs, or modified cells, such as modified lymphocytes, modified TILs, modified lymphocytes other than B cells, or modified T cells and/or modified NK cells, wherein such biologic includes any nucleic acid(s) or other component(s) disclosed herein. Such container in illustrative embodiments includes substantially pure polynucleotides, such as polynucleotide vectors, for example RIPs, or modified cells, such as modified lymphocytes, modified TILs, modified lymphocytes other than B cells, or modified T cells and/or modified NK cells, sometimes referred to herein for shorthand, as a substantially biologic. Typically, a preparation and/or container of substantially pure retroviral particles is sterile, and negative for mycoplasma, replication competent retroviruses of the same type, and adventitious viruses according to standard protocols (see e.g., “Viral Vector Characterization: A Look at Analytical Tools”; Oct. 10, 2018 (available at https://cellculturedish.com/viral-vector-characterization-analytical-tools/)). Exemplary methods for generating substantially pure biologics and cells for cell therapy are known. For example, for such methods with respect to RIPs, viral supernatants can be purified by a combination of depth filtration, TFF, benzonase treatment, diafiltration, and formulation. In certain illustrative embodiments, a substantially pure biologic meets all of the following characteristics based on quality control testing results:
a) negative for mycoplasma;
b) endotoxin at less than 25 EU/ml, and in certain further illustrative embodiments, less than 10 EU/ml;
c) absence of replication competent retroviruses detected of the same type as purposefully in the container (e.g., lentiviruses) detected; and
d) absence of adventitious viruses detected.

Furthermore, if the biologic is a viral particle, such as a retroviral particle, in exemplary embodiments, it meets the following quality control testing results:
a) less than 1 pg host cell DNA/transducing unit (TU), and in certain further illustrative embodiments, less than 0.3 pg/TU;
b) less than 100 residual plasmid copies/TU, and in certain further illustrative embodiments, less than 10 copies/TU of any plasmid used to make the recombinant retroviral particles;
c) less than 1 ng host cell protein/TU, and in certain further illustrative embodiments, less than 50 pg host cell protein/TU; and/or
d) greater than 100 TU/ng p24 protein, and in certain further illustrative embodiments, greater than 10,000 TU/ng p24 protein.

Retroviral particles are typically tested against release specifications that include some or all of those provided above, before they are released to a customer. Mycoplasma testing can be performed in accordance with The United States Pharmacopeia's (USP) chapter <63> or by a rapid method such as RT-PCR of samples for the absence mycoplasma. Endotoxin testing can be performed in accordance with USP chapter <85> or a rapid method such as Endosafe® nexgen-PTS™ from Charles River. Replication competent retrovirus can be tested by assaying for reverse transcriptase activity by qPCR-based Product Enhanced Reverse Transcriptase (PERT) assay or a rapid RCL assay. Adventitious virus detection can be accomplished in vitro by inoculating indicator cells lines (MRC-5, Vero, HEK 293T) with test articles, observing the cultures for cytopathic effects for 14 days, and testing for hemagglutination and hemadsorption of red blood cells. Host cell DNA can be quantitated by qPCR for host cell genomic nucleic acid. Residual viral RNA genome copies can be measured by q-RT-PCR for nucleic acid residues of viral envelope protein such as VSVG. Host cell protein can be measured by ELISA using polyclonal antibodies against a host cell lysate. P24 analysis for determination of physical titer based on HIV-1-based lentiviral supernatant can be determined by standard ELISA.

Potency of each particle may be defined on the basis of p24 viral capsid protein or viral RNA genome copies and can be converted to infectious titer by measuring functional gene transfer Transducing Units (TUs) in a bioassay.

Determination of infectious titer of purified bulk retrovirus material and finished product by bioassay and qPCR is an exemplary analytical test method for the determination of infectious titer of retroviruses. An indicator cell bank (such as Jurkat or F1XT(a HEK293T cell variant)) may be seeded at 150,000 cells per well, followed by exposure to serial dilutions of the retrovirus product. Dilutions of purified retrovirus particles are made on indicator cells, for example from 1:200 to 1:1,600. A reference standard virus may be added for system suitability. Following 4 days (or 2 to 4 days) of incubation with retrovirus, the cells are harvested, DNA extracted and purified. A standard curve, for example from 100-10,000,000 copies/well, of human genome and unique retroviral genome sequence plasmid pDNA amplicons are used followed by addition of genomic DNA of the cell samples exposed to retrovirus particles. For each PCR reaction, the Cq values of both the retrovirus amplicon and the endogenous control such as human RNAseP are extrapolated back to copies per reaction. From these values the integrated genome copy number is calculated. In some cases, indicator cells such as HEK 293T have been characterized as being triploid, hence 3 copies of a single copy gene per cell should be utilized in the calculation. Using the initial viable cell count per well, the volume of retrovirus added to the cells and the genome copy number ratio, a Transducing Unit (TU) per ml retrovirus particles may be determined. 1 TU is the amount of functional viral particles in a solution that generates 1 transgene integration when the viral solution is added to 100 permissive cells.

Potency testing can include potency testing against release specifications with purity and specific activity. For example, potency release testing of final product can include measurement of the number of Transducing Units (TU) compared to viral particle quantity (e.g., by performing an ELISA against a viral protein, for example, for lentivirus by performing a p24 capsid protein ELISA with a cutoff of at least 100, 1,000, 2,000 or 2,500 TU/ng p24), and CAR functionality, for example by measuring interferon gamma release by a reporter cell line exposed to gene modified cells. Thus, RIP formulations herein can have potency or potency range equal to any of the potency testing cutoffs and ranges herein.

The purity of replication incompetent recombinant retroviral particles (RIPs) can be determined using the ratio of the amount of protein from the host cells used to generate the of RIPs to the transducing units (amount host cell protein/TU). In some embodiments, the ratio of host cell protein to TUs can be 10, 5, 3, 2, or 1 ng or less host cell protein/TU or 750, 500, 400, 300, 200, 100, 50, 40, 30, 20, or 10 μg or less host cell protein/TU. In some embodiments, the ratio of host cell protein to TUs can be 1 ng or less host cell protein/TU. In some embodiments, the ratio of host cell protein to TUs can be 50 μg or less host cell protein/TU. In some embodiments, the host cell is a HEK cell line or variant thereof. In some embodiments, the ratio of HEK protein to TUs can be 10, 5, 3, 2, or 1 ng or less HEK protein/TU or 750, 500, 400, 300, 200, 100, 50, 40, 30, 20, or 10 μg or less HEK protein/TU. In some embodiments, the ratio of HEK protein to Tus can be 1 ng or less protein/TU. In some embodiments, the ratio of HEK protein to Tus can be 50 μg or less HEK protein/TU.

The potency of RIPs present in a delivery solution or RIP formulation can be determined using the ratio of the TUs to the ng of p24 protein. In some embodiments, the ratio of the TUs to the ng of p24 protein can be 100, 200, 300, 400, 500, 1,000, 4,000, 10,000, 12,500, or 15,000 or more TUs/ng of p24 protein.

In some embodiments, the purity and potency of RIPs in a substantially pure biologic can be any of the combinations provided in the Exemplary Embodiments section herein.

In some embodiments, the quality control testing can include the ratios of host cell protein/TU and the TU/ng p24 protein being, respectively: 1 ng host cell protein/TU or less and 100 TU/ng p24 protein or more; 1 ng host cell protein/TU or less and 500 TU/ng p24 protein or more; 1 ng host cell protein/TU or less and 1,000 TU/ng p24 protein or more; 1 ng host cell protein/TU or less and 5,000 TU/ng p24 protein or more; 1 ng host cell protein/TU or less and 10,000 TU/ng p24 protein or more; 1 ng host cell protein/TU or less and 12,500 TU/ng p24 protein or more; 1 ng host cell protein/TU or less and 15,000 TU/ng p24 protein or more; 50 pg host cell protein/TU or less and 100 TU/ng p24 protein or more; 50 pg host cell protein/TU or less and 500 TU/ng p24 protein or more; 50 pg host cell protein/TU or less and 1,000 TU/ng p24 protein or more; 50 pg host cell protein/TU or less and 5,000 TU/ng p24 protein or more; 50 pg host cell protein/TU or less and 10,000 TU/ng p24 protein or more; 50 pg host cell protein/TU or less and 12,500 TU/ng p24 protein or more; or 50 pg host cell protein/TU or less and 15,000 TU/ng p24 protein or more.

In some embodiments, the host cell can be a HEK 293 cell line or variant thereof including a HEK 293T cell line. In such embodiments, the quality control testing can include the ratios of HEK protein/TU and TU/ng p24 protein being, respectively: 1 ng HEK protein/TU or less and 100 TU/ng p24 protein or more; 1 ng HEK protein/TU or less and 500 TU/ng p24 protein or more; 1 ng HEK protein/TU or less and 1,000 TU/ng p24 protein or more; 1 ng HEK protein/TU or less and 5,000 TU/ng p24 protein or more; 1 ng HEK protein/TU or less and 10,000 TU/ng p24 protein or more; 1 ng HEK protein/TU or less and 12,500 TU/ng p24 protein or more; 1 ng HEK protein/TU or less and 15,000 TU/ng p24 protein or more; 50 pg HEK protein/TU or less and 100 TU/ng p24 protein or more; 50 pg HEK protein/TU or less and 500 TU/ng p24 protein or more; 50 pg HEK protein/TU or less and 1,000 TU/ng p24 protein or more; 50 pg HEK protein/TU or less and 5,000 TU/ng p24 protein or more; 50 pg HEK protein/TU or less and 10,000 TU/ng p24 protein or more; 50 pg HEK protein/TU or less and 12,500 TU/ng p24 protein or more; or 50 pg HEK protein/TU or less and 15,000 TU/ng p24 protein or more.

In any of the kit or isolated replication incompetent recombinant retroviral particle aspects herein, that include a container of such retroviral particles, sufficient recombinant retroviral particles are present in the container to achieve an MOI (the number of Transducing Units, or TUs applied per cell) in a reaction mixture made using the retroviral particles, of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15. The Transducing Units of virus particles provided in the kit should enable the use an MOI that prevents producing too many integrants in an individual cell, on average less than 5, 4, or 3 lentigenome copies per cellular genome and more preferably 1 copy per cell. For kit and isolated retroviral particle embodiments, such MOI can be based on 1, 2.5, 5, 10, 20, 25, 50, 100, 250, 500, or 1,000 ml of reaction mixture assuming 1×10⁶ target cells/ml, for example in the case of whole blood, assuming 1×10⁶ PBMCs/ml of blood. Accordingly, a container of retroviral particles can include between 1×10⁵ and 1×10⁹, 1×10⁵ and 1×10⁸⁰ 1×10⁵ and 5×10⁷, 1×10⁵ and 1×10⁷, 1×10⁵ and 1×10⁶; 5×10⁵ and 1×10⁹; 5×10⁵ and 1×10⁸, 5×10⁵ and 5×10⁷, 5×10⁵ and 1×10⁷, 5×10⁵ and 1×10⁶, or 1×10⁷ and 1×10⁹, 1×10⁷ and 5×10⁷, 1×10⁶ and 1×10⁷, and 1×10⁶ and 5×10⁶ TUs. In certain illustrative embodiments, the container can contain between 1×10⁷ and 1×10⁹, 5×10⁶ and 1×10⁸, 1×10⁶ and 5×10⁷, 1×10⁶ and 5×10⁶ or between 5×10⁷ and 1×10⁸ retroviral Transducing Units. Not to be limited by theory, such numbers of particles would support between 1 and 100 ml of blood at an MOI of between 1 and 10. In some illustrative embodiments, as indicate herein, as little as 10 ml, 5 ml, 3 ml, or even 2.5 ml of blood can be processed for T cell and/or NK cell modification and optionally subcutaneous and/or intramuscular administration methods provided herein. Thus, an advantage of the present methods is that in some illustrative embodiments, they require far fewer retroviral particle Transducing Units than prior methods that involve nucleic acids encoding a CAR, such as CAR-T methods.

Each container that contains retroviral particles, can have, for example, a volume of between 0.05 ml and 5 ml, 0.05 ml and 1 ml, 0.05 ml and 0.5 ml, 0.1 ml and 5 ml, 0.1 ml and 1 ml, 0.1 ml and 0.5 ml, 0.1 and 10 ml, 0.5 and 10 ml, 0.5 ml and 5 ml, 0.5 ml and 1 ml, 1.0 ml and 10.0 ml, 1.0 ml and 5.0 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml, 0.25 ml to 10 ml, 0.25 to 5 ml, or 0.25 to 2 ml.

In certain embodiments, retroviral particles in the container are GMP-grade, or cGMP-grade retroviral particles (i.e., produced under GMP or current GMP requirements according to a regulatory agency), or the product of a retroviral manufacturing process performed using GMP systems. Such retroviral particles are typically made using a USA FDA (i.e., U.S. GMP or U.S. cGMP), EMA (i.e., EMA GMP or EMA cGMP), or National Medical Products Administration (NMPA) of China (i.e., Chinese FDA) (i.e., NMPA GMP or NMPA cGMP) good manufacturing practice (GMP), for example using GMP quality systems and GMP procedural controls. These products are typically produced in facilities that meet GMP or cGMP requirements. Such products are typically manufactured under a strict quality management system based on GMP or cGMP regulations. GMP-grade retroviral particles are typically sterile. This can be accomplished for example, by filtering retroviral particles, for example substantially pure retroviral particles, with a 0.45 μm or a 0.22 μm filter. GMP-grade retroviral particles are typically substantially pure, and prepared with control manufacturing test specifications for potency, quality and safety.

In some embodiments, the solution comprising retroviral particles in the container is free of detectable bovine proteins, which can be referred to as “bovine-free”. For example, such solution of retroviral particles can be bovine free because bovine proteins, such as bovine serum proteins, are not used in culturing the packaging cells during retrovirus production. In some embodiment, the solution of retroviral particles is GMP-grade and bovine-free. Substantially pure nucleic acid solutions are typically bovine-free and manufactured in bovine-free broth.

With respect to retroviral particles, and in illustrative embodiments, lentiviral particles, in certain exemplary reaction mixtures provided herein, between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15, multiplicity of infection (MOI); or at least 1 and less than 6, 11, or 51 MOI; or in some embodiments, between 5 and 10 MOI units of replication incompetent recombinant retroviral particles are present. In some embodiments, the MOI can be at least 0.1, 0.5, 1, 2, 2.5, 3, 5, 10 or 15. With respect to composition and method for transducing lymphocytes in blood, in certain embodiments higher MOI can be used than in methods wherein PBMCs are isolated and used in the reaction mixtures. For example, illustrative embodiments of compositions and methods for transducing lymphocytes in whole blood, assuming 1×10⁶ PBMCs/ml of blood, can use retroviral particles with an MOI of between 1 and 50, 2 and 25, 2.5 and 20, 2.5 and 10, 4 and 6, or about 5, and in some embodiments between 5 and 20, 5 and 15, 10 and 20, or 10 and 15.

Surface expression of the TCR complex, including TCRα, TCRβ, and CD3, on CD4 positive (CD4+) cells and CD8 positive (CD8+) cells is reduced or “dimmed” when such cells are contacted with polynucleotide vectors (e.g., replication incompetent recombinant (RIR) retroviral particles) displaying a binding polypeptide that binds the TCR complex, e.g., a T cell activation element, as is the case in certain illustrative embodiments herein. This dimming is largely the result of internalization of the TCR complex upon activation. Furthermore, the extent of this dimming increases as the concentration of a given gene vector is increased in the reaction mixture and correlates with the ability of the gene vector to activate and enter cells. Similarly, internalization of other surface polypeptides after binding to polypeptides on the surface of a gene vector results in dimming of the surface polypeptide on the cell being contacted with the gene vector and may be common during transduction using other binding polypeptides. Thus, in some embodiments, a percent reduction in surface polypeptide expression on cells contacted with a gene vector comprising a binding polypeptide compared to surface polypeptide expression on cells not contacted with the gene vector comprising a binding polypeptide is used to quantitate the potency of a gene vector and determine the appropriate dose of gene vector used to modify a population of cells. In illustrative embodiments, a percent reduction in surface TCR complex expression on cells contacted with a gene vector compared to surface TCR complex expression on cells not contacted with the gene vector is used to quantitate the potency of a gene vector and determine the appropriate dose of gene vector used to modify a population of cells. As used herein, a “Dimming Unit” (DU) is the amount of gene vector (e.g., RIR retroviral particles) that reduces the surface expression of a surface polypeptide in 1 ml of a cell mixture after contacting with the gene vector for 4 hours at 37° C. and 5% CO₂ by 50% compared to the surface expression of the surface polypeptide in the cell mixture under similar conditions but not contacted with the gene vector. The surface polypeptide is typically a binding partner of a binding polypeptide present on the surface of the gene vector. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the binding partner is CD3 and the binding polypeptide is anti-CD3.

Because the level of expression of a binding polypeptide on the surface of a polynucleotide vector (sometimes referred to herein as a “gene vector”) will vary between different binding polypeptides and between polynucleotide vector preparations, the ability of a polynucleotide vector to reduce surface expression of a surface polypeptide should be determined for each preparation of a polynucleotide vector. In some embodiments, the ability of a polynucleotide vector to reduce surface expression of a surface polypeptide is determined based on target cell number. In some embodiments, the ability of a polynucleotide vector to reduce surface expression of a surface polypeptide is based on the volume the cells. In any of the aspects and embodiments herein, the reduction of surface expression of a surface polypeptide can be referred to as dimming the surface polypeptide. For example, if the surface expression of CD3 on a cell is reduced, then CD3 is dimmed on that cell and the cell can be called CD3−, even though the cell may still contain CD3 not expressed on its surface. Not to be limited by theory, T cells that temporarily internalize and dim CD3 are T cells and will eventually re-express CD3 on their cell surfaces such that they are again CD3+.

Accordingly, provided herein in one aspect, is a method for determining an amount of a polynucleotide vector preparation to dim surface expression of a surface polypeptide by a dimming percentage on cells in a dimming volume, comprising:
a) forming a plurality of reaction mixtures comprising a plurality of volumes of the polynucleotide vector preparation and a plurality of volumes of a cell mixture, wherein at least two of the reaction mixtures in the plurality of reaction mixtures comprise different volumes of the polynucleotide vector preparation and/or the cell mixture, wherein the cell mixture comprises a plurality of cells comprising the surface polypeptide on their surfaces, and wherein the polynucleotide vector preparation comprises a plurality of polynucleotide vectors comprising a binding polypeptide on their surfaces capable of binding the surface polypeptide;
b) incubating the reaction mixtures;
c) measuring the surface expression of the surface polypeptide in the reaction mixtures and in an uncontacted volume of the cell mixture, wherein the uncontacted volume of the cell mixture is not contacted with the polynucleotide vector preparation; and
d) determining the amount of the polynucleotide vector preparation to dim the dimming percentage of cells in the dimming volume the measured surface expression of the surface polypeptide in the reaction mixtures, the measured surface expression of the surface polypeptide in the uncontacted volume of the cell mixture, and the amounts of the polynucleotide vector preparation and the cell mixture in the reaction mixtures.

In some embodiments, the amount of the cell mixture in the reaction mixtures is based on volume. In some embodiments, the amount of the cell mixture in the reaction mixtures is based on numbers of target cells. In some embodiments, the polynucleotide vector preparation is a viral preparation. In illustrative embodiments, the viral preparation is a replication incompetent recombinant retroviral particle preparation. In some embodiments, the dimming percentage (percentage of cells dimmed) is 50%, 60%, 70%, 75%, 80%, 85%, 90%, 95%, 96%, or 97%. In illustrative embodiments, the dimming percentage is at least or about 80%, 85%, 90%, or 95%. In some embodiments, the dimming volume is 0.25 ml, 0.5 ml, 0.75 ml, 1 ml, 2 ml, 3 ml, 4 ml, 5 ml, 10 ml, 15 ml, 20 ml, or 25 ml. In some embodiments, the surface polypeptide can be CD3D, CD3E, CD3G, CD3Z, TCRα, TCRβ, CD16A, NKp46, 2B4, CD2, DNAM, or NKG2C, NKG2D, NKG2E, NKG2F, and/or NKG2H. In some embodiments, the surface polypeptide is a TCR complex polypeptide. In some embodiments, the TCR complex polypeptide is CD3D, CD3E, CD3G, CD3Z, TCRα, or TCRβ. In illustrative embodiments, the surface polypeptide is CD3E. In some embodiments, the binding polypeptide can be any of the activation elements disclosed in the Activation Elements section herein. In such embodiments, the surface polypeptide can be the binding partner of the activation element.

In illustrative embodiments, the cell mixture is whole blood. In further illustrative embodiments, the cell mixture has been subjected to a red blood cell depletion procedure. In some embodiments, the whole blood is collected from a healthy subject, e.g., a subject that does not have or is not known or suspected to have a disease, disorder, or condition associated with an elevated expression of an antigen. In some embodiments, the whole blood is collected from a subject with a disease, disorder, or condition associated with an elevated expression of an antigen, wherein the polynucleotide vector will be administered to the subject or other subjects with the disease disorder, or condition. In some embodiments, the whole blood is collected from each subject and the Dimming Units are calculated for each subject individually.

In some embodiments, the reaction mixtures can be incubated for less than or about 24, 12, 10, 8, 6, 4, or 2 hours or 60, 45, 30, 15, 10, or 5 minutes, or for just an initial contacting. In some embodiments, the reaction mixtures can be incubated for between 10 minutes and 24 hours, or between 10 minutes and 8 hours, or for between 1 hour and 8 hours, or for between 1 hour and 6 hours, or in illustrative embodiments, for between 3.5 and 4.5 hours or for 4 hours. In some embodiments, the reaction mixtures can be incubated at about 10° C., 15° C., 20° C., 25° C., 30° C., 37° C., or 42° C. In some embodiments, the reaction mixtures are incubated without CO₂. In illustrative embodiments, the reaction mixtures are incubated with 5% CO₂.

In some embodiments, the surface expression of the surface polypeptide is measured by a fluorescence-activated cell sorting (FACS) method. In some embodiments, the antibody used in a FACS method is GMP. In some embodiments, a CD3 antibody is used to determine surface expression of the surface polypeptide. In some embodiments, the CD3 antibody is UCHT1, OKT-3, HIT3A, TRX4, X35-3, VIT3, BMA030 (BW264/56), CLB-T3/3, CRIS7, YTH12.5, F111409, CLB-T3.4.2, TR-66, TR66.opt, HuM291, WT31, WT32, SPv-T3b, 11D8, XIII-141, XIII46, XIII-87, 12F6, T3/RW2-8C8, T3/RW24B6, OKT3D, M-T301, SMC2, F101.01, and/or SK7. In illustrative embodiments, the CD3 antibody is PerCP Mouse Anti-Human CD3—Clone SK7 (BD, 347344). In some embodiments, before measuring the surface expression of the surface polypeptide, cells present in the cell mixture are separated from unbound polynucleotide vector in the incubated reaction mixture.

In an illustrative embodiment of the above method, the polynucleotide vector preparation is a RIP preparation, the dimming percentage is 50%, the dimming volume is 1 ml, the surface polypeptide is CD3, the cell mixture is whole blood collected from a healthy subject, and the reaction mixture is incubated for 4 hours at 37° C. and 5% CO₂ and the method is used to calculate Dimming Units.

Such methods can be used to determine the amount of retroviral particles in a polynucleotide vector preparation that reduces surface polypeptide expression on cells by a specific percentage. This amount can then be used to determine an amount of the preparation of retroviral particles to use for subsequent transductions of whole blood, isolated PBMCs, or isolated TNCs. In any of the aspects and embodiments provided herein that include genetically modifying and/or transducing lymphocytes, the amount of a preparation of the polynucleotide vector, for example replication incompetent recombinant retroviral particles, to add to the lymphocytes can be determined using the method above.

Dimming Units (DUs) can be used in any of the aspects or embodiments herein that include a contacting step to determine the amount of the polynucleotide vector to add. As 1 DU of the polynucleotide vector reduces the surface expression of the surface polypeptide by 50% in a 1 ml volume of cells, 10 DUs of the polynucleotide vector reduces the surface expression of the surface polypeptide by 50% in 10 ml of a cell mixture. In some embodiments, sufficient DUs are added to a volume of cells to reduce surface expression of the surface polypeptide, for example CD3, by greater than 50%, 60%, 70%, 75%, 80%, 85%, 90%, 95%, 96%, or 97% after contacting with the polynucleotide vector compared to the surface expression of the surface polypeptide in the cell mixture under similar conditions but not contacted with the polynucleotide vector. In illustrative embodiments, sufficient DUs are added to a volume of cells to reduce surface expression of the surface polypeptide by greater than 80%, 85%, 90%, or 95% after contacting with the polynucleotide vector compared to the surface expression of the surface polypeptide in the cell mixture under similar conditions but not contacted with the polynucleotide vector. In some embodiments, at least 1, 2, 3, 4, 5, 6, 7, 8, 9, 10, 11,12, 13, 14, 15, 16, 17, 18, 19 or 20 DU are added per ml of cell mixture. In illustrative embodiments, between 5 and 20 DU, 5 and 15 DU, 10 and 20 DU, or 13 and 18 DU are added per ml of cell mixture. In some embodiments, at least 1, 2, 3, 4, 5, 6, 7, 8, 9, 10, 11,12, 13, 14, 15, 16, 17, 18, 19 or 20 DU are added per 1,000,000 target cells. In some embodiments, the target cells are lymphocytes, for example T cells or NK cells. In illustrative embodiments, the cells are in whole blood, isolated PBMCs, or isolated TNCs. In further illustrative embodiments, the cells are the remaining fraction of whole blood after lysing red blood cells. In some embodiments, sufficient DUs are added to dim a population of cells a specific percentage, for example, to dim CD3 on a population of T cells by greater than 50%, 60%, 70%, 75%, 80%, 85%, 90%, 95%, 96%, or 97%. In some embodiments, sufficient dimming units of a polynucleotide vector, and in illustrative embodiments RIP are present to increase the percentage of surface dimmed surface polypeptide, and in illustrative embodiments dimmed surface CD3−, in a population of cells, and in illustrative embodiments T cells, to at least 50%, 60%, 70%, 75%, 80%, 85%, 90%, 95%, 96%, or 97%. In any of the aspects and embodiments herein that include cells contacted with a polynucleotide vector, the composition including cells can include at least 1, 2, 3, 4, 5, 6, 7, 8, 9, 10, 11,12, 13, 14, 15, 16, 17, 18, 19 or 20 DU per ml of the cells, for example at least 1, 2, 3, 4, 5, 6, 7, 8, 9, 10, 11,12, 13, 14, 15, 16, 17, 18, 19 or 20 DU per ml of blood, cell formulation, or population of cells.

In some aspects, provided herein is a kit for modifying NK cells and/or, in illustrative embodiments, T cells. Such a kit in certain embodiments, includes one or a plurality of containers containing polynucleotides, typically substantially pure polynucleotides comprising one or more first transcriptional units operatively linked to a promoter active in T cells and/or NK cells, wherein the one or more first transcriptional units encode a first polypeptide comprising a first chimeric antigen receptor (CAR), sometimes referred to as a first CAR, and one or more containers of accessory component(s), also called accessory kit components herein. The polynucleotides (e.g., retroviral particles) can be stored frozen, for example at −70° C. or lower (e.g., −80° C.).

In some embodiments where the kit includes modified cells, such as modified lymphocytes, modified TILs, modified lymphocytes that are not B cells, such as modified T cells and/or modified NK cells in a cell suspension within a commercial container, for example a cryopreservation infusion bag, the container, such as the cryopreservation infusion bag, can hold 5, 10, 15, 20, 25, 50, 75, 100, 150, 200, 250, 300, 400, or 500 ml or less of blood. In some embodiments, the container, for example the cryopreservation infusion bag, can hold at least 5, 10, 15, 20, 25, 50, 75, 100, 150, 200, 250, 300, 400, or 500 ml of blood. In some embodiments, the container, for example the cryopreservation infusion bag, can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. In some embodiments, the container, for example the cryopreservation infusion bag, can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. For example, the container, for example the cryopreservation infusion bag, can hold between 1 and 10 ml, 5 and 25 ml, 10 and 50 ml, 25 and 100 ml, 50 and 200 ml, or 100 and 500 ml of blood. In some embodiments, the container, for example the cryopreservation infusion bag, can include heparin. In other embodiments, the container, for example the cryopreservation infusion bag, does not include heparin.

In some embodiments where the kit includes modified cells, such as a modified lymphocytes, modified TILs, modified lymphocytes other than B cells, or modified T cells and/or modified NK cells in a cell suspension within a commercial container, such as a cell cryopreservation infusion bag, the number of cells delivered can be sufficient to provide between 1×10⁵ cells and 1×10⁹, between 1×10⁶ cells and 1×10⁹, or between 1×10⁶ cells and 5×10⁸, for example CAR-positive viable T cells and/or NK cells per kg of body weight of the subject to which the cells are to be delivered. Accordingly, in some embodiments the commercial container can include the aforementioned ranges×50-150 kg, or 50-100 kg. In some embodiments, the commercial container includes between 1×10⁷ and 1×10¹¹ cells, between 1×10⁸ and 1×10¹¹ cells, or between 1×10⁸ and 5×10¹⁰ cells, for example CAR-positive viable T cells and/or NK cells, or in an illustrative embodiment, cells that are positive for an anti-idiotype extracellular recognition domain.

In illustrative embodiments, the polynucleotides encoding any of the various polypeptides disclosed herein, e.g., a CAR, are located in the genome of retroviral particles, typically substantially pure retroviral particles, according to any of the replication incompetent recombinant retroviral particle aspects and embodiments provided herein. In illustrative embodiments, the replication incompetent recombinant retroviral particles in the kit comprise a polynucleotide comprising one or more transcriptional units operatively linked to a promoter active in T cells and/or NK cells, wherein the one or more first transcriptional units encode a first polypeptide comprising an anti-idiotype polypeptide, a CAR, a TCR, and/or an LE and optionally encode a second polypeptide comprising an anti-idiotype polypeptide, a CAR, a TCR, and/or an LE, according to any of the embodiments provided herein.

A kit provided herein can include a container containing the polynucleotides, such as polynucleotide vectors, for example RIPs, or modified cells, such as, modified lymphocytes, modified TILs, modified lymphocytes other than B cells, for example modified T cells and/or NK cells, and an accessory kit. The accessory kit components can include one or more of the following:
a. one or more containers containing a delivery solution compatible with, in illustrative embodiments effective for, and in further illustrative embodiments adapted for subcutaneous and/or intramuscular administration as provided herein;
b. one or more containers of hyaluronidase as provided herein;
c. one or more blood bags such as a blood collection bag, in illustrative embodiments comprising an anticoagulant in the bag, or in a separate container, a blood processing buffer bag, a blood processing waste collection bag, and a blood processing cell sample collection bag;
d. one or more sterile syringes compatible with, in illustrative embodiments effective for, and in further illustrative embodiments adapted for, subcutaneous or intramuscular delivery of T cells and/or NK cells;
e. a T cell activation element as disclosed in detail herein, for example anti-CD3 provided in solution in the container containing the retroviral particle, or in a separate container, or in illustrative embodiments, is associated with a surface of the replication incompetent retroviral particle;
f. one or a plurality of leukoreduction filtration assemblies;
g. one or more containers containing a solution or media compatible with, in illustrative embodiments effective for, and in further illustrative embodiments adapted for transduction of T cells and/or NK cells;
h. one or more containers containing a solution or media compatible with, in illustrative embodiments effective for, and/or in further illustrative embodiments adapted for rinsing T cells and/or NK cells;
i. one or more containers containing a pH-modulating pharmacologic agent;
j. one or more containers containing polynucleotides, typically substantially pure polynucleotides (e.g., found within recombinant retroviral particles according to any embodiment herein), comprising one or more second transcriptional units operatively linked to a promoter active in T cells and/or NK cells, wherein the one or more second transcriptional units encode a polypeptide comprising a second CAR directed against a different target epitope, and in certain embodiments a different antigen, in illustrative embodiments found on a same target cancer cell (e.g., B cell);
k. one or more containers containing a cognate antigen for the first CAR and/or the second CAR encoded by the nucleic acids (e.g., retroviral particles); and
l. Instructions, either physically or digitally associated with other kit components, for the use thereof, for example for modifying T cells and/or NK cells, for delivering modified T cells and/or NK cells to a subject subcutaneously or intramuscularly, and/or for treating tumor growth or cancer in a subject.

In some embodiments, the blood bags can hold 5, 10, 15, 20, 25, 50, 75, 100, 150, 200, 250, 300, 400, or 500 ml or less of blood. In some embodiments, the blood bags can hold at least 5, 10, 15, 20, 25, 50, 75, 100, 150, 200, 250, 300, 400, or 500 ml of blood. In some embodiments, the blood bags can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. In some embodiments, the blood bag can hold between 1, 2, 3, 4, 5, 10, 15, 20, 25, and 50 ml of blood on the low end of the range and 10, 15, 20, 25, 50, 75, 100, 150, 200, 250, 300, 400, and 500 ml of blood on the high end of the range. For example, the blood bag can hold between 1 and 10 ml, 5 and 25 ml, 10 and 50 ml, 25 and 100 ml, 50 and 200 ml, or 100 and 500 ml of blood. In some embodiments, the blood bags can include heparin. In other embodiments, the blood bags do not include heparin.

In any of the kit aspects and embodiments herein that include an antigen or a cognate antigen, less than 50%, 40%, 30%, 20%, 10%, 5%, or 1% of the polypeptides in the kit are non-human, i.e., produced from non-human sources.

In some embodiments, the kit may be a single-pack/use kit, but in other embodiments the kit is a multi-pack or multi-use kit for the processing of more than one blood sample from contacting with nucleic acids encoding a CAR optionally thru subcutaneous administration. Typically, a container of nucleic acids encoding a CAR (and optionally a paired container of nucleic acids encoding a second CAR in certain embodiments) in the kit is used for one performance of a method for modifying T cells and/or NK cells and optionally subcutaneous administration. The container(s) containing nucleic acids encoding a CAR and optionally a second CAR is typically stored and shipped frozen. Thus, a kit can include sufficient containers (e.g., vials) of nucleic acids encoding a CAR (and optionally paired containers encoding a second CAR in certain embodiments) for 1, 2, 3, 4, 5, 6, 10, 12, 20, 24, 50 and 100 performances of a method for modifying a T cell and/or NK cell provided herein, and thus can include 1, 2, 3, 4, 5, 6, 10, 12, 20, 24, 50 and 100 containers (e.g., vials) of nucleic acids encoding the CAR (e.g., retroviral particles), and similarly is considered a 1, 2, 3, 4, 5, 6, 10, 12, 20, 24, 50 and 100 pack, performance, administration or X kit, respectively. Similarly, accessory components in the kit would be provided for similar numbers of performances of a method for modifying T cells and/or NK cells and optionally subcutaneous administration, using the kit.

The one or more leukoreduction filtration assemblies, if present in such a kit, typically include(s) one or a plurality of leukoreduction filters or leukoreduction filter sets, each typically within a filter enclosure, as well as a plurality of connected sterile tubes connected or adapted to be connected thereto, and a plurality of valves connected or adapted to be connected thereto, that are adapted for use in a single-use closed blood processing system. Typically, there is one leukoreduction filtration assembly for each container of nucleic acid encoding a CAR in a kit. Thus a 20-pack kit in illustrative embodiments, includes 20 vials of nucleic acids encoding a CAR and 20 leukoreduction filtration assemblies. In some embodiments, a kit herein comprises one or a plurality of containers containing nucleic acids and one or more leukoreduction filtration assemblies. Such a kit can optionally be intended to be used for administration to a subject via any route including for example, infusion or in illustrative embodiments intramuscular and/or in further illustrative embodiments, subcutaneous delivery. Thus, such a kit optionally includes other accessory components that are intended to be used with such route of administration. The one or more containers of subcutaneous or intramuscular delivery solution is discussed in more detail herein, is typically sterile and can include a total combined volume, or individually per container, of 100 ml to 5 L, 1 ml to 1 L, 1 ml to 500 ml, 1 ml to 250 ml, 1 ml to 200 ml, 1 ml to 100 ml, 1 ml to 10 ml, or 1 ml to 5 ml; 5 ml to 1 L, 5 ml to 500 ml, 5 ml to 250 ml, 5 ml to 100 ml, 5 ml to 10 ml, or approximately 5 ml. In some illustrative embodiments, the kit comprises a plurality of containers of subcutaneous delivery solution, with each container having a volume of between 10 ml and 200 ml, 10 ml and 100 ml, 1 ml and 20 ml, 1 ml and 10 ml, 1 ml and 5 ml, 1 ml and 2 ml, 2 ml and 20 ml, 2 ml and 10 ml, 2 ml and 5 ml, 0.25 ml to 10 ml, 0.25 to 5 ml, or 0.25 to 2 ml. In illustrative embodiments, there is one container of delivery solution for each container of nucleic acid encoding a CAR in a kit. Thus, a 20-pack kit in illustrative embodiments, includes 20 vials of nucleic acids encoding a CAR and 20 containers of sterile delivery solution.

In certain kit aspects, provided herein are embodiments in which either or both the container(s) containing nucleic acids encoding a first CAR and optionally nucleic acids encoding a second CAR. In such embodiments, accessory components of the kit can further include one or more of the following:
a. one or more containers containing a delivery solution adapted for, compatible with, and/or effective for, intravenous or intraperitoneal administration as provided herein; and
b. Instructions, either physically or digitally associated with other kit components, for the use thereof, for example for delivering modified T cells and/or NK cells to a subject intravenously or intraperitoneally.

In certain aspects, provided herein are the use of a RIP in the manufacture of a kit for modifying a T cell or NK cell, wherein the use of the kit includes: contacting the T cell or NK cell ex vivo with the replication incompetent recombinant retroviral particle, wherein the replication incompetent recombinant retroviral particle includes a pseudotyping element on a surface and a T cell activation element on the surface, wherein said contacting facilitates transduction of the T cell or NK cell by the replication incompetent recombinant retroviral particle, thereby producing a modified and in illustrative embodiments genetically modified T cell or NK cell.

In some aspects, provided herein are aspects that include the use of a replication incompetent recombinant retroviral particle in the manufacture of a kit for modifying a T cell or NK cell. Details regarding polynucleotides, and replication incompetent recombinant retroviral particles that contain such polynucleotides are disclosed in more detail herein, and in the Exemplary Embodiments section. In some embodiments, the T cell or NK cell can be from a subject. In some embodiments, the T cell activation element can be membrane-bound. In some embodiments, the contacting can be performed for between 1, 2, 3, 4, 5, 6, 7, or 8 hours on the low end of the range and 4, 5, 6, 7, 8, 10, 12, 15, 18, 21, and 24 hours on the high end of the range, for example, between 1 and 12 hours. The replication incompetent recombinant retroviral particle for use in the manufacture of a kit can include any of the aspects, embodiments, or sub-embodiments discussed elsewhere herein.

Furthermore, provided herein in another aspect is a container, such as a commercial container or package, or a kit comprising the same, comprising isolated packaging cells, in illustrative embodiments isolated packaging cells from a packaging cell line, according to any of the packaging cell and/or packaging cell line aspects provided herein. In some embodiments, the kit includes additional containers that include additional reagents such as buffers or reagents used in methods provided herein. Furthermore, provided herein in certain aspects are use of any replication incompetent recombinant retroviral particle provided herein in any aspect, in the manufacture of a kit for modifying and in illustrative embodiments genetically modifying a T cell or NK cell according to any aspect provided herein. Furthermore, provided herein in certain aspects are use of any packaging cell or packaging cell line provided herein in any aspect, in the manufacture of a kit for producing the replication incompetent recombinant retroviral particles according to any aspect provided herein.

In another aspect, provided herein is a pharmaceutical composition for treating or preventing cancer or tumor growth comprising a replication incompetent recombinant retroviral particle as an active ingredient. In another aspect, provided herein is an infusion composition or other cell formulation for treating or preventing cancer or tumor growth comprising a replication incompetent recombinant retroviral particle. The replication incompetent recombinant retroviral particle of the pharmaceutical composition or infusion composition can include any of the aspects, embodiments, or subembodiments discussed above or elsewhere herein.

### Genes of Interest

A viral particle of the present disclosure, which in illustrative embodiments is a retroviral particle, typically includes a polynucleotide that includes one or more genes of interest (GOIs). The GOIs can be any GOI known in the art. In some embodiments, a GOI can encode a hyaluronidase polypeptide (as disclosed elsewhere herein), an engineered signaling polypeptide (e.g., a CAR, TCR and/or lymphoproliferative element as disclosed elsewhere herein), a checkpoint-inhibiting ligand, a nucleic acid sequence or polypeptide that stimulates an immune response towards the modified cell, a nucleic acid sequence or polypeptide that stimulates apoptosis, a nucleic acid sequence or polypeptide that functions as a transporter or is a member of a transporter construct, a nucleic acid sequence or polypeptide that stimulates growth (e.g., VEGF, GM-CSF), a toxic protein, a nucleic acid sequence or polypeptide that stimulates autophagy and shutdown of protein synthesis, a nucleic acid sequence or polypeptide that stimulates induction of anti-tumoral immunity, a suicide gene (e.g., cytocidal dominant negative cyclin-G1 construct and herpes simplex virus thymidine kinase (HSV-TK)), a safety switch (e.g., recognition and/or elimination domain), an anti-idiotype polypeptide, any polypeptide known to be deleted or defective in diseases, CRISPR-Cas, guide RNAs, and/or an inhibitory RNA, for example, inhibitory RNA to a GOI. A skilled artisan will understand that a polynucleotide in a viral particle can include multiple GOIs, which can be expressed as fusion polypeptides, separated by a cleavage signal or ribosomal skip sequence such as P2A or T2A, or have separate IRES sequences.

A skilled artisan will understand that the gene of interest can be expressed at different stages during the transduction of the target cell. In some embodiments, the polynucleotide can be designed to express the gene of interest directly from the RNA or DNA present in the viral particle. In some embodiments, RNA present in the viral particle can be reverse-transcribed into DNA before expression of the gene of interest in the target cell. In some embodiments, DNA present in the viral particle or DNA generated from reverse-transcribing the viral RNA can be integrated into the genome of the target cell before expression of the gene of interest.

In illustrative embodiments, the viral particle is to be administered in vivo to a subject or patient, such that the cells that will express the gene of interest are not isolated or enriched before the transduction. To transduce the appropriate target cells, the viral particles include a targeting element, which binds the viral particle to the target cell. Thus, a GOI is present in a viral particle that includes on its surface a targeting element, as discussed elsewhere herein, that will deliver the GOI to the appropriate target cells.

A skilled artisan will under that various diseases can be treated by delivering a gene of interest. In some embodiments, a GOI is delivered to a subject or patient, wherein the subject or patient has spinal muscular atrophy, hemophilia, Duchenne muscular dystrophy, polyneuropathy of hereditary transthyretin amyloidosis (hATTR), hyperlipoproteinaemia, amyotrophic lateral sclerosis (ALS), Huntington disease, cancer (including any cancer types disclosed elsewhere herein, and including relapsed and/or refractory cancers), familial partial lipodystrophy (FPL), Leber congenital amaurosis, retinal dystrophy (including biallelic retinal pigment epithelium-specific 65 kilodalton (RPE65) mutation-associated retinal dystrophy), adrenoleukodystrophy, and/or cystic fibrosis.

In some embodiments, the GOI can include SMN1 (survival motor neuron 1), SMN2 (also known as BCD541, C-BCD541, GEMIN1, SMNC, TDRD16B, survival motor neuron 2), F8 (also known as AHF, DXS1253E, F8B, F8C, FVIII, HEMA, THPH13), a DMD gene that is amenable to exon 51 skipping, DMD gene located on chromosome Xp21, ATTR, RPE65, human interferon alpha-2b, ABCD1, ARSA (arylsulfatase A), DDC (dopa decarboxylase, also known as AADC, AAAD, tryptophan decarboxylase, 5-hydroxytryptophan decarboxylase), tyrosine hydroxylase (TH, also known as DYT14, DYT5b), GTPCH (GTP-cyclohydrolase 1, also known as DYT14, DYT5, DYT5a, GCH, GTP-CH-1, GTPCH1, HPABH4B, GTP cyclohydrolase 1), ABCR (Retina-Specific ABC Transporter, also known as ABCA4), MYO7A (also known as Usher Syndrome Type 1B gene), p53 (also known as TP53, LFS1, Antigen NY-CO-13, BMFS5, TRP53, BCC7) VEGF (vascular endothelial growth factor, also known as VPF), GMCSF (granulocyte-macrophage colony-stimulating factor, also known as CSF2 (colony-stimulating factor 2), G-CSF (granulocyte-colony stimulating factor, also known as CSF3 (colony stimulating factor 3), HGF (hepatocyte growth factor, also known as HPTA, SF, F-TCF), and/or CFTR.

### Engineered Signaling Polypeptide(s)

In some embodiments, the replication incompetent recombinant retroviral particles used to contact T cells and/or NK cells, whether in vivo (e.g., in direct RIP administration aspects and embodiments) or ex vivo, have a polynucleotide or nucleic acid having one or more transcriptional units that encode one or more engineered signaling polypeptides. In some embodiments, an engineered signaling polypeptide includes any combination of an extracellular domain (e.g., an antibody, an antigen-specific targeting region or ASTR), a stalk and a transmembrane domain, combined with one or more intracellular activating domains, optionally one or more modulatory domains (such as a co-stimulatory domain), and optionally one or more T cell survival motifs. In illustrative embodiments, at least one, two, or all of the engineered signaling polypeptides is a chimeric antigen receptor (CAR) or a lymphoproliferative element (LE) such as a chimeric lymphoproliferative element (CLE). In some embodiments, at least one, two, or all of the engineered signaling polypeptides is an engineered T cell receptor (TCR). In some embodiments, when two signaling polypeptides are utilized, one encodes a lymphoproliferative element and the other encodes a chimeric antigen receptor (CAR) that includes an antigen-specific targeting region (ASTR), a transmembrane domain, and an intracellular activating domain. For any domain of an engineered signaling polypeptide disclosed herein, exemplary sequences can be found in WO2019/055946, incorporated herein in its entirety by reference. A skilled artisan will recognize that such engineered polypeptides can also be referred to as recombinant polypeptides. The engineered signaling polypeptides, such as CARs, engineered TCRs, LEs, and CLEs provided herein, are typically transgenes with respect to lymphocytes, especially T cells and NK cells, and most especially T cells and/or NK cells that are engineered using methods and compositions provided herein, to express such signaling polypeptides.

### Extracellular Domain

In some embodiments, an engineered signaling polypeptide includes an extracellular domain that is a member of a specific binding pair. For example, in some embodiments, the extracellular domain can be the extracellular domain of a cytokine receptor, or a mutant thereof, or a hormone receptor, or a mutant thereof. Such mutant extracellular domains in some embodiments have been reported to be constitutively active when expressed at least in some cell types. In illustrative embodiments, such extracellular and transmembrane domains do not include a ligand binding region. It is believed that such domains do not bind a ligand when present in an engineered signaling polypeptide and expressed in B cells, T cells, and/or NK cells. Mutations in such receptor mutants can occur in the extracellular juxtamembrane region. Not to be limited by theory, a mutation in at least some extracellular domains (and some extracellular-transmembrane domains) of engineered signaling polypeptides provided herein, are responsible for signaling of the engineered signaling polypeptide in the absence of ligand, by bringing activating chains together that are not normally together. Further embodiments regarding extracellular domains that comprise mutations in extracellular domains can be found, for example, in the Lymphoproliferative Element section herein.

In certain illustrative embodiments, the extracellular domain comprises a dimerizing motif. In an illustrative embodiment the dimerizing motif comprises a leucine zipper. In some embodiments, the leucine zipper is from a jun polypeptide, for example c-jun. Further embodiments regarding extracellular domains that comprise a dimerizing motif can be found, for example, in the Lymphoproliferative Element section herein.

In certain embodiments, the extracellular domain is an antigen-specific targeting region (ASTR), sometimes called an antigen binding domain herein. Specific binding pairs include, but are not limited to, antigen-antibody binding pairs; ligand-receptor binding pairs; and the like. Thus, a member of a specific binding pair suitable for use in an engineered signaling polypeptide of the present disclosure includes an ASTR that is an antibody, an antigen, a ligand, a receptor binding domain of a ligand, a receptor, a ligand binding domain of a receptor, and an alternative non-antibody scaffold, also referred to herein as an antibody mimetic. In any of the aspects or embodiments provided herein that include an ASTR, the ASTR can be a suitable antibody mimetic. In some embodiments, the antibody mimetic can be an affibody, an afflilin, an affimer, an affitin, an alphabody, an alphamab, an anticalin, an armadillo repeat protein, an atrimer, an avimer (also known as avidity multimer), a C-type lectin domain, a cysteine-knot miniprotein, a cyclic peptide, a cytotoxic T-lymphocyte associated protein-4, a DARPin (Designed Ankyrin Repeat Protein), a fibrinogen domain, a fibronectin binding domain (FN3 domain) (e.g., adnectin or monobody), a fynomer, a knottin, a Kunitz domain peptide, a leucine-rich repeat domain, a lipocalin domain, a mAb 2 or Fcab™, a nanobody, a nanoCLAMP, an OBody, a Pronectin, a single-chain TCR, a tetratricopeptide repeat domain, or a V-like domain. In any of the aspects or embodiments provided herein that include an ASTR that is an antibody, for example, an scFv, a suitable antibody mimetic can be used instead of the antibody.

An ASTR suitable for use in an engineered signaling polypeptide of the present disclosure can be any antigen-binding polypeptide. In certain embodiments, the ASTR is an antibody such as a full-length antibody, a single-chain antibody, a Fab fragment, a Fab′ fragment, a (Fab′)2 fragment, a Fv fragment, and a divalent single-chain antibody or a diabody.

In some embodiments, the ASTR is a single chain Fv (scFv). In some embodiments, the heavy chain is positioned N-terminal of the light chain in the engineered signaling polypeptide. In other embodiments, the light chain is positioned N-terminal of the heavy chain in the engineered signaling polypeptide. In any of the disclosed embodiments, the heavy and light chains can be separated by a linker as discussed in more detail herein. In any of the disclosed embodiments, the heavy or light chain can be at the N-terminus of the engineered signaling polypeptide and is typically C-terminal of another domain, such as a signal sequence or peptide.

Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use with the engineered signaling polypeptides and methods using the engineered signaling polypeptides of the present disclosure. In some instances, T cell receptor (TCR) based recognition domains.

Naturally-occurring T cell receptors include an α-subunit and a β-subunit, separately produced by unique recombination events in a T cell's genome. Libraries of TCRs may be screened for their selectivity to a target antigen, for example, any of the antigens disclosed herein. Screens of natural and/or engineered TCRs can identify TCRs with high avidities and/or reactivities towards a target antigen. Such TCRs can be selected, cloned, and a polynucleotide encoding such a TCR can be included in a replication incompetent recombinant retroviral particle to genetically modify a lymphocyte, or in illustrative embodiments, T cell or NK cell, such that the lymphocyte expresses the engineered TCR. In some embodiments, the TCR can be a single chain TCR (scTv, single chain two-domain TCR containing VaVD).

Certain embodiments for any aspect or embodiment herein that includes a CAR, include CARs having extracellular domains engineered to co-opt the endogenous TCR signaling complex and CD3Z signaling pathway. In one embodiment, a chimeric antigen receptor ASTR is fused to one of the endogenous TCR complex chains (e.g., TCR alpha, CD3E etc.) to promote incorporation into the TCR complex and signaling through the endogenous CD3Z chains. In other embodiments, a CAR contains a first scFv or protein that binds to the TCR complex and a second scFv or protein that binds to the target antigen (e.g., tumor antigen). In another embodiment, the TCR can be a single chain TCR (scTv, single chain two-domain TCR containing VaVp). Finally, scFv's may also be generated to recognize the specific MHC/peptide complex, thereby acting as a surrogate TCR. Such peptide/MHC scFv-binders may be used in many similar configurations as CARs.

In some embodiments, the ASTR can be multispecific, e.g., bispecific antibodies. Multispecific antibodies have binding specificities for at least two different sites. In certain embodiments, one of the binding specificities is for one target antigen and the other is for another target antigen. In certain embodiments, bispecific antibodies may bind to two different epitopes of a target antigen. Bispecific antibodies may also be used to localize cytotoxic agents to cells which express a target antigen. Bispecific antibodies can be prepared as full-length antibodies or antibody fragments.

An ASTR suitable for use in an engineered signaling polypeptide of the present disclosure, or an engineered TCR, can have a variety of antigen-binding specificities. In some cases, the antigen-binding domain is specific for an epitope present in an antigen that is expressed by (synthesized by) a target cell. In one example, the target cell is a cancer cell associated antigen. The cancer cell associated antigen can be an antigen associated with, e.g., a breast cancer cell, a B cell lymphoma cell, as a diffuse large B cell lymphoma (DLBCL) cell, a Hodgkin lymphoma cell, an ovarian cancer cell, a prostate cancer cell, a mesothelioma, a lung cancer cell (e.g., a small cell lung cancer cell), a lymphoma cell, a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a leukemia cell, a chronic myelogenous leukemia (CML) cell, a chronic lymphocytic leukemia (CLL) cell, an acute myelogenous leukemia (AML) cell, an acute lymphocytic leukemia (ALL) cell, a neuroblastoma cell, a glioma, a glioblastoma, a medulloblastoma, a colorectal cancer cell, etc. A cancer cell associated antigen may also be expressed by a non-cancerous cell. In some embodiments, the cancer cell is a PDL-1 positive cancer cell. In illustrative embodiments, the cancer cell is a PDL-1 positive DLBCL cell. In some embodiments, the cancer cell is a PDL-1 negative cell. In illustrative embodiments, the cancer cell is a PDL-1 negative DLBCL cell.

In any of the aspects or embodiments herein that include an ASTR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD5, CD7, CD19, CD20, CD22, CD23, CD24, CD27, CD30, CD33, CD34, CD37, CD38, CD40, CD44, CD44v6, CD44v7/8, CD45, CD70, CD99, CD117, CD123, CD133, CD138, CD171, CS1, GD2 (ganglioside G2), EphA2, CSPG4, FAP (Fibroblast Activation Protein), kappa, lambda, 5T4, πvβ6 integrin, integrin πvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, c-Met, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), PDL-1, GPC3, GUCY2C, HER1, ICAM-1, IL13Rα2, IL11Rα, Kras, Kras G12D, LlCAM, MET, MUC1, MUC16 ecto, NKG2D, NY-ESO-1, PSCA, WT-1 and the like.

In some embodiments, a member of a specific binding pair suitable for use in an engineered signaling polypeptide is an ASTR that is a ligand for a receptor. Ligands include, but are not limited to, hormones (e.g., erythropoietin, growth hormone, leptin, etc.); cytokines (e.g., interferons, interleukins, certain hormones, etc.); growth factors (e.g., heregulin; vascular endothelial growth factor (VEGF); and the like); an integrin-binding peptide (e.g., a peptide comprising the sequence Arg-Gly-Asp (SEQ ID NO:1)); and the like.

Where the member of a specific binding pair in an engineered signaling polypeptide is a ligand, the engineered signaling polypeptide can be activated in the presence of a second member of the specific binding pair, where the second member of the specific binding pair is a receptor for the ligand. For example, where the ligand is VEGF, the second member of the specific binding pair can be a VEGF receptor, including a soluble VEGF receptor.

As noted above, in some cases, the member of a specific binding pair that is included in an engineered signaling polypeptide is an ASTR that is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); CD27; a natural cytotoxicity receptor (NCR) (e.g., NKP30 (NCR3/CD337) polypeptide (receptor for HLA-B-associated transcript 3 (BAT3) and B7-H6); etc.); etc.

In certain embodiments of any of the aspects provided herein that include an ASTR, the ASTR can be directed to an intermediate protein that links the ASTR with a target molecule expressed on a target cell. The intermediate protein may be endogenously expressed or introduced exogenously and may be natural, engineered, or chemically modified. In certain embodiments the ASTR can be an anti-tag ASTR such that at least one tagged intermediate, typically an antibody-tag conjugate, is included between a tag recognized by the ASTR and a target molecule, typically a protein target, expressed on a target cell. Accordingly, in such embodiments, the ASTR binds a tag and the tag is conjugated to an antibody directed against an antigen on a target cell, such as a cancer cell. Non-limiting examples of tags include fluorescein isothiocyanate (FITC), streptavidin, biotin, histidine, dinitrophenol, peridinin chlorophyll protein complex, green fluorescent protein, phycoerythrin (PE), horse radish peroxidase, palmitoylation, nitrosylation, alkaline phosphatase, glucose oxidase, and maltose binding protein. As such, the ASTR comprises a molecule that binds the tag.

### Stalk

In some embodiments, the engineered signaling polypeptide includes a stalk which is located in the portion of the engineered signaling polypeptide lying outside the cell and interposed between the ASTR and the transmembrane domain. Stalks are known in the art and a skilled artisan will understand how to incorporate them into engineered signaling polypeptides. In some embodiments, the stalk has at least 85, 90, 95, 96, 97, 98, 99, or 100% identity to a wild-type CD8 stalk region (TTTPAPRPPTPAPTIASQPLSLRPEACRPAAGG AVHTRGLDFA (SEQ ID NO:2), has at least 85, 90, 95, 96, 97, 98, 99, or 100% identity to a wild-type CD28 stalk region (FCKIEVMYPPPYLDNEKSNGTIIHVKGKHLCPSPLFPGPSKP (SEQ ID NO:3)), or has at least 85, 90, 95, 96, 97, 98, 99, or 100% identity to a wild-type immunoglobulin heavy chain stalk region. In an engineered signaling polypeptide, the stalk employed allows the antigen-specific targeting region, and typically the entire engineered signaling polypeptide, to retain increased binding to a target antigen.

The stalk region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

In some embodiments, the stalk of an engineered signaling polypeptide includes at least one cysteine. For example, in some embodiments, the stalk can include the sequence Cys-Pro-Pro-Cys (SEQ ID NO:4). If present, a cysteine in the stalk of a first engineered signaling polypeptide can be available to form a disulfide bond with a stalk in a second engineered signaling polypeptide.

Stalks can include immunoglobulin hinge region amino acid sequences that are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include a domain with at least 50, 60, 70, 75, 80, 85, 90, 95, 96, 97, 98, 99 or 100% sequence identity to a stretch of at least 10, 15, 20, or all of the amino acids of any of the following amino acid sequences: DKTHT (SEQ ID NO:5); CPPC (SEQ ID NO:4); CPEPKSCDTPPPCPR (SEQ ID NO:6) (see, e.g., Glaser et al. (2005) J. Biol. Chem. 280:41494); ELKTPLGDTTHT (SEQ ID NO:7); KSCDKTHTCP (SEQ ID NO:8); KCCVDCP (SEQ ID NO:9); KYGPPCP (SEQ ID NO: 10); EPKSCDKTHTCPPCP (SEQ ID NO: 11) (human IgG1 hinge); ERKCCVECPPCP (SEQ ID NO: 12) (human IgG2 hinge); ELKTPLGDTTHTCPRCP (SEQ ID NO: 13) (human IgG3 hinge); SPNMVPHAH1HAQ (SEQ ID NO: 14) (human IgG4 hinge); and the like. The stalk can include a hinge region with an amino acid sequence of a human IgG1, IgG2, IgG3, or IgG4, hinge region. The stalk can include one or more amino acid substitutions and/or insertions and/or deletions compared to a wild-type (naturally-occurring) hinge region. For example, His229 of human IgG 1 hinge can be substituted with Tyr, so that the stalk includes the sequence EPKSCDKTYTCPPCP (SEQ ID NO: 15), (see, e.g., Yan et al. (2012) J. Biol. Chem. 287:5891). The stalk can include an amino acid sequence derived from human CD8; e.g., the stalk can include the amino acid sequence: TTTPAPRPPTPAPTIASQPLSLRPEACRPAAGGAVHTRGLDFACD (SEQ ID NO: 16), or a variant thereof.

### Transmembrane Domain

An engineered signaling polypeptide of the present disclosure can include transmembrane domains for insertion into a eukaryotic cell membrane. The transmembrane domain can be interposed between the ASTR and the co-stimulatory domain. The transmembrane domain can be interposed between the stalk and the co-stimulatory domain, such that the chimeric antigen receptor includes, in order from the amino terminus (N-terminus) to the carboxyl terminus (C-terminus): an ASTR; a stalk; a transmembrane domain; and an activating domain. Transmembrane domains are known in the art and a skilled artisan will understand how to incorporate them into engineered signaling polypeptides.

Any transmembrane (TM) domain that provides for insertion of a polypeptide into the cell membrane of a eukaryotic (e.g., mammalian) cell is suitable for use in aspects and embodiments disclosed herein. In some embodiments, the TM domain for any aspect provided herein that includes a CAR can include atransmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD27, CD16, CD18, CD19, CD22, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof.

Non-limiting examples of™ domains suitable for any of the aspects or embodiments provided herein, include a domain with at least 50, 60, 70, 75, 80, 85, 90, 95, 96, 97, 98, 99 or 100% sequence identity to a stretch of at least 10, 15, 20, or all of the amino acids of any of the following TM domains or combined stalk and TM domains: a) CD8 alpha TM (SEQ ID NO: 17); b) CD8 beta TM (SEQ ID NO: 18); c) CD4 stalk (SEQ ID NO: 19); d) CD3Z TM (SEQ ID NO:20); e) CD28 TM (SEQ ID NO:21); f) CD134 (OX40) TM: (SEQ ID NO:22); g) CD7 TM (SEQ ID NO:23); h) CD8 stalk and TM (SEQ ID NO:24); and i) CD28 stalk and TM (SEQ ID NO:25).

As non-limiting examples, a transmembrane domain of an aspect of the present disclosure can have at least 80%, 90%, or 95% or can have 100% sequence identity to the SEQ ID NO: 17 transmembrane domain, or can have 100% sequence identity to any of the transmembrane domains from the following genes respectively: the CD8 beta transmembrane domain, the CD4 transmembrane domain, the CD3 zeta transmembrane domain, the CD28 transmembrane domain, the CD134 transmembrane domain, or the CD7 transmembrane domain.

### Intracellular Activating Domain

Intracellular activating domains suitable for use in an engineered signaling polypeptide of the present disclosure when activated, typically induce the production of one or more cytokines; increase cell death; and/or increase proliferation of CD8⁺ T cells, CD4⁺ T cells, NKT cells, γδ T cells, and/or neutrophils. Activating domains can also be referred to as activation domains herein. Activating domains can be used in CARs or in lymphoproliferative elements provided herein. Intracellular activating domains are known in the art and a skilled artisan will understand how to incorporate them into engineered signaling polypeptides.

In some embodiments, the intracellular activating domain includes at least one (e.g., one, two, three, four, five, six, etc.) ITAM motifs as described below. In some embodiments, an intracellular activating domain of an aspect of the present disclosure can have at least 80%, 90%, or 95% or can have 100% sequence identity to the CD3Z, CD3D, CD3E, CD3G, CD79A, CD79B, DAP12, FCER1G, FCGR2A, FCGR2C, DAP10/CD28, ZAP70, NKp30 (B7-H6), NKG2D, NKp44, NKp46, FcR gamma (FCER1G), FcR beta (FCER1B), FcgammaRL, FcgammaRIIA, FcgammaRIIC, FcgammaRIIIA, and FcRL5 domains as described below.

Intracellular activating domains suitable for use in an engineered signaling polypeptide of the present disclosure include immunoreceptor tyrosine-based activation motif (ITAM)-containing intracellular signaling polypeptides. An ITAM motif is YX₁×₂L/I, where X₁ and X₂ are independently any amino acid. In some embodiments, the intracellular activating domain of an engineered signaling polypeptide includes 1, 2, 3, 4, or 5 ITAM motifs. In some embodiments, an ITAM motif is repeated twice in an intracellular activating domain, where the first and second instances of the ITAM motif are separated from one another by 6 to 8 amino acids, e.g., (YX₁×₂L/I)(X₃)˜(YX₁×₂L/I), where n is an integer from 6 to 8, and each of the 6-8 X₃ can be any amino acid. In some embodiments, the intracellular activating domain of an engineered signaling polypeptide includes 3 ITAM motifs.

A suitable intracellular activating domain can be an ITAM motif-containing portion that is derived from a polypeptide that contains an ITAM motif. For example, a suitable intracellular activating domain can be an ITAM motif-containing domain from any ITAM motif-containing protein. Thus, a suitable intracellular activating domain need not contain the entire sequence of the entire protein from which it is derived. Examples of suitable ITAM motif-containing polypeptides include, but are not limited to: CD3Z (CD3 zeta); CD3D (CD3 delta); CD3E (CD3 epsilon); CD3G (CD3 gamma); CD79A (antigen receptor complex-associated protein alpha chain); CD79B (antigen receptor complex-associated protein beta chain) DAP12; and FCER1G (Fc epsilon receptor I gamma chain).

In some embodiments, an intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids in the following ITAM motif-containing polypeptides or to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of any of the following ITAM motif-containing polypeptides: CD3 zeta chain (also known as CD3Z, T cell receptor T3 zeta chain, CD247, CD3-ZETA, CD3H, CD3Q, T3Z, TCRZ, etc.) with exemplary sequences MKWKALFTAAILQAQLPITEAQSFGLLDPKLCYLLDGILFIYGVILTALFLRVKFSRSADAPAYQQ GQNQL[YNELNLGRREEYDVL]DKRRGRDPEMGGKPRRKNPQEGL[YNELQKDKMAEAYSEI]G MKGERRRGKGHDGL[YQGLSTATKDTYDAL]HMQALPPR (SEQ ID NO:26), MKWKALFTAAILQAQLPITEAQSFGLLDPKLCYLLDGILFIYGVILTALFLRVKFSRSADAPAYQQ GQNQL[YNELNLGRREEYDVL]DKRRGRDPEMGGKPQRRKNPQEGL[YNELQKDKMAEAYSEI]GMKGERRRGKGHDGL[YQGLSTATKDTYDAL]HMQALPPR (SEQ ID NO:27); RVKFSRSADAPAYQQGQNQL[YNELNLGRREEYDVL]DKRRGRDPEMGGKPRRKNPQEGL[YNE LQKDKMAEAYSEI]GMKGERRRGKGHDGL[YQGLSTATKDTYDAL]HMQALPPR (SEQ ID NO:28), RVKFSRSADAPAYQQGQNQL[YNELNLGRREEYDVL]DKRRGRDPEMGGKPQRRKNPQEGL[YN ELQKDKMAEAYSEI]GMKGERRRGKGHDGL[YQGLSTATKDTYDAL]HMQALPPR (SEQ ID NO:29), NQL[YNELNLGRREEYDVL]DKR SEQ ID NO:30); EGL[YNELQKDKMAEAYSEI]GMK (SEQ ID NO:31), and DGL[YQGLSTATKDTYDAL]HMQ (SEQ ID NO:32); T cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T cell receptor T3 delta chain; T cell surface glycoprotein CD3 delta chain; etc.) with exemplary sequences: MEHSTFLSGLVLATLLSQVSPFKIPIEELEDRVFVNCNTSITWVEGTVGTLLSDITRLDLGKRILDP RGIYRCNGTDIYKDKESTVQVHYRMCQSCVELDPATVAGIIVTDVIATLLLALGVFCFAGHETGR LSGAADTQALLRNDQV[YQPLRDRDDAQYSHL]GGNWARNK (SEQ ID NO:33), MEHSTFLSGLVLATLLSQVSPFKIPIEELEDRVFVNCNTSITWVEGTVGTLLSDITRLDLGKRILDP RGIYRCNGTDIYKDKESTVQVHYRTADTQALLRNDQV[YQPLRDRDDAQYSHL]GGNWARNK (SEQ ID NO:34), and DQV[YQPLRDRDDAQYSHL]GGN (SEQ ID NO:35); T cell surface glycoprotein CD3 epsilon chain (also known as CD3e, T cell surface antigen T3/Leu-4 epsilon chain, T cell surface glycoprotein CD3 epsilon chain, A1504783, CD3, CD3epsilon, T3e, etc.) with exemplary sequences: MQSGTHWRVLGLCLLSVGVWGQDGNEEMGGITQTPYKVSISGTTVILTCPQYPGSEILWQHNDK NIGGDEDDKNIGSDEDHLSLKEFSELEQSGYYVCYPRGSKPEDANFYLYLRARVCENCMEMDMS VATIVIVDICITGGLLLLVYYWSKNRKAKAKPVTRGAGAGGRQRGQNKERPPPVPNPD[YEPIRK GQRDLYSGL]NQRRI (SEQ ID NO:36) and NPD[YEPIRKGQRDLYSGL]NQR (SEQ ID NO:37); T cell surface glycoprotein CD3 gamma chain (also known as CD3G, T cell receptor T3 gamma chain, CD3-GAMMA, T3G, gamma polypeptide (TiT3 complex), etc.) with exemplary sequences: MEQGKGLAVLILAIILLQGTLAQSIKGNHLVKVYDYQEDGSVLLTCDAEAKNITWFKDGKMIGF LTEDKKKWNLGSNAKDPRGMYQCKGSQNKSKPLQVYYRMCQNCIELNAATISGFLFAEIVSIFV LAVGVYFIAGQDGVRQSRASDKQTLLPNDQL[YQPLKDREDDQYSHL]QGNQLRRN (SEQ ID NO:38) and DQL[YQPLKDREDDQYSHL]QGN (SEQ ID NO:39); CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.) with exemplary sequences: MPGGPGVLQALPATIFLLFLLSAVYLGPGCQALWMHKVPASLMVSLGEDAHFQCPHNSSNNAN VTWWRVLHGNYTWPPEFLGPGEDPNGTLIIQNVNKSHGGIYVCRVQEGNESYQQSCGTYLRVR QPPPRPFLDMGEGTKNRIITAEGIILLFCAVVPGTLLLFRKRWQNEKLGLDAGDEYEDENL[YEGL NLDDCSMYEDI]SRGLQGTYQDVGSLNIGDVQLEKP (SEQ ID NO:40), MPGGPGVLQALPATIFLLFLLSAVYLGPGCQALWMHKVPASLMVSLGEDAHFQCPHNSSNNAN VTWWRVLHGNYTWPPEFLGPGEDPNEPPPRPFLDMGEGTKNRIITAEGIILLFCAVVPGTLLLFRK RWQNEKLGLDAGDEYEDENL[YEGLNLDDCSMYEDI]SRGLQGTYQDVGSLNIGDVQLEKP (SEQ ID NO:41), and ENL[YEGLNLDDCSMYEDI]SRG (SEQ ID NO:42); CD79B with exemplary sequence: LDKDDSKAGMEEDHT[YEGLDIDQTATYEDI]VTLRTGEVKWSVGEHPGQE (SEQ ID NO:211); DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.) with exemplary sequences: MGGLEPCSRLLLLPLLLAVSGLRPVQAQAQSDCSCSTVSPGVLAGIVMGDLVLTVLIALAVYFLG RLVPRGRGAAEAATRKQRITETESP[YQELQGQRSDVYSDL]NTQRPYYK (SEQ ID NO:43), MGGLEPCSRLLLLPLLLAVSGLRPVQAQAQSDCSCSTVSPGVLAGIVMGDLVLTVLIALAVYFLG RLVPRGRGAAEATRKQRITETESP[YQELQGQRSDVYSDL]NTQ (SEQ ID NO:44), MGGLEPCSRLLLLPLLLAVSDCSCSTVSPGVLAGIVMGDLVLTVLIALAVYFLGRLVPRGRGAAE AATRKQRITETESP[YQELQGQRSDVYSDL]NTQRPYYK (SEQ ID NO:45), MGGLEPCSRLLLLPLLLAVSDCSCSTVSPGVLAGIVMGDLVLTVLIALAVYFLGRLVPRGRGAAE ATRKQRITETESP[YQELQGQRSDVYSDL]NTQRPYYK (SEQ ID NO:46), and ESP[YQELQGQRSDVYSDL]NTQ (SEQ ID NO:47); and FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.) with exemplary sequences: MIPAVVLLLLLLVEQAAALGEPQLCYILDAILFLYGIVLTLLYCRLKIQVRKAAITSYEKSDGV[YT GLSTRNQETYETL]KHEKPPQ (SEQ ID NO:48) and DGV[YTGLSTRNQETYETL]KHE (SEQ ID NO:49), where the ITAM motifs are set out in brackets.

Intracellular activating domains suitable for use in an engineered signaling polypeptide of the present disclosure include a DAP10/CD28 type signaling chain. An example of a DAP10 signaling chain is the amino acid SEQ ID NO:50. In some embodiments, a suitable intracellular activating domain includes a domain with at least 50%, 60%, 70%, 75%, 80%, 85%, 90%, 95%, 96%, 97%, 98%, 99% or 100% sequence identity to a stretch of at least 10, 15, 20, or all amino acids in SEQ ID NO:50.

An example of a CD28 signaling chain is the amino acid sequence is SEQ ID NO:51. In some embodiments, a suitable intracellular domain includes a domain with at least 50%, 60%, 70%, 75%, 80%, 85%, 90%, 95%, 96%, 97%, 98%, 99% or 100% sequence identity to a stretch of at least 10, 15, 20, or all amino acids of SEQ ID NO:51.

Intracellular activating domains suitable for use in an engineered signaling polypeptide of the present disclosure include a ZAP70 polypeptide. For example, a suitable intracellular activating domain can include a domain with at least 50%, 60%, 70%, 75%, 80%, 85%, 90%, 95%, 96%, 97%, 98%, 99% or 100% sequence identity to a stretch of at least 10, 15, 20, or all amino acids in the following sequences or to a contiguous stretch of from about 300 amino acids to about 400 amino acids, from about 400 amino acids to about 500 amino acids, or from about 500 amino acids to 619 amino acids, of SEQ ID NO:52.

### Modulatory Domains

Modulatory domains can change the effect of the intracellular activating domain in the engineered signaling polypeptide, including enhancing or dampening the downstream effects of the activating domain or changing the nature of the response. Modulatory domains are known in the art and a skilled artisan will understand how to incorporate them into engineered signaling polypeptides. Modulatory domains suitable for use in an engineered signaling polypeptide of the present disclosure include co-stimulatory domains. A modulatory domain suitable for inclusion in the engineered signaling polypeptide can have a length of from about 30 amino acids to about 70 amino acids (aa), e.g., a modulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, modulatory domain can have a length of from about 70 aa to about 100 aa, from about 100 aa to about 200 aa, or greater than 200 aa.

Co-stimulatory domains typically enhance and/or change the nature of the response to an activation domain. Co-stimulatory domains suitable for use in an engineered signaling polypeptide of the present disclosure are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). In some embodiments, any of the CAR provided herein can include a costimulatory domain. In some embodiments, the co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, 25, 30, 35, 40, 45, or 50 amino acids or an intracellular domain of 4-1BB (CD137), B7-H3, B7-HCDR3, BAFFR, BTLA, C100 (SEMA4D), CD2, CD4, CD7, CD8A, CD8B, CD11A, CD11B, CD11C, CD11D, CD18, CD19, CD27, CD28, CD28 deleted for Lek binding (ICA), CD29, CD30, CD40, CD49A, CD49D, CD49F, CD69, CD84, CD96 (Tactile), CD103, CD160 (BY55), CD162 (SELPLG), CD226 (DNAM1), CD229 (Ly9), a ligand that specifically binds with CD83, CDS, CEACAM1, CRLF2, CRTAM, CSF2RA, CSF2RB, CSF3R, EPOR, Fc receptor gamma chain, Fc receptor E chain, FCGRA2, GADS, GHR, GITR, HVEM, IA4, ICAM-1, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, ILSRA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA4, ITGA6, ITGAD, ITGAE, ITGAL, ITGAM, ITGAX, ITGB1, ITGB2, ITGB7, LAT, LEPR, LFA-1 (CD11a/CD18), LIGHT, LIFR, LMP1, LTBR, MPL, MYD88, NKG2C, NKP80 (KLRF1), OSMR, OX40, PD-1, PRLR, PSGL1, PAG/Cbp, SLAM (SLAMF1, CD150, IPO-3), SLAMF4 (C244, 2B4), SLAMF6 (NTB-A, Ly108), SLAMF7, SLAMF8 (BLAME), SLP-76, TILR2, TILR4, TILR7, TILR9, TNFR2, TNFRSF4, TNFRSF8, TNFRSF9, TNFRSF14, TNFRSF18, TRANCE/RANKL, VLA1, or VLA-6, or functional mutants and/or fragments thereof.

A co-stimulatory domain suitable for inclusion in an engineered signaling polypeptide can have a length of from about 30 amino acids to about 70 amino acids (aa), e.g., a co-stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, the co-stimulatory domain can have a length of from about 70 aa to about 100 aa, from about 100 aa to about 200 aa, or greater than 200 aa.

In some embodiments, a co-stimulatory domain can include a domain with at least 50%, 60%, 70%, 75%, 80%, 85%, 90%, 95%, 96%, 97%, 98%, 99% or 100% sequence identity to a stretch of at least 10, 15, 20, or all the amino acids or from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa (depending on how long the intracellular portion of the protein is) of an intracellular portion of: CD137 (also known as TNFRSF9; CD137; 4-1BB; CDw137; ILA; etc.) for example SEQ ID NO:53, CD28 (also known as Tp44) for example SEQ ID NO:54, CD28 deleted for Lek binding (ICA) for example SEQ ID NO:55, ICOS (also known as AILIM, CD278, and CVIDl) for example SEQ ID NO:56, OX40 (also known as TNFRSF4, RP5-902P8.3, ACT35, CD134, OX-40, TXGPIL) for example SEQ ID NO:57, CD27 (also known as S 152, T 14, TNFRSF7, and Tp55) for example SEQ ID NO:58, BTLA (also known as BTLA1 and CD272) for example SEQ ID NO:59, CD30 (also known as TNFRSF8, D1S166E, and Ki-1), for example SEQ ID NO:60, GITR (also known as TNFRSF18, RP5-902P8.2, AITR, CD357, and GITR-D), for example SEQ ID NO:61, or HVEM (also known as TNFRSF14, RP3-395M20.6, ATAR, CD270, HVEA, HVEM, LIGHTR, and TR2), for example SEQ ID NO:62. OX40 contains a p85 PI3K binding motif at residues 34-57 and a TRAF binding motif at residues 76-102, each of SEQ ID NO: 296 (of Table 1). In some embodiments, a co-stimulatory domain can include a sequence with at least 80% sequence identity to a stretch of at least 15 amino acids of SEQ ID NOs:53-62 and 296. In some embodiments, a co-stimulatory domain can include a sequence with at least 90% sequence identity to a stretch of at least 15 amino acids of SEQ ID NOs:53-62 and 296. In some embodiments, a co-stimulatory domain can include a sequence with at least 95% sequence identity to a stretch of at least 15 amino acids of SEQ ID NOs:53-62 and 296. In some embodiments, the costimulatory domain can include the p85 PI3K binding motif of OX40. In some embodiments, the costimulatory domain can include the TRAF binding motif of OX40. Lysines corresponding to amino acids 17 and 41 of SEQ ID NO: 296 are potentially negative regulatory sites that function as parts of ubiquitin targeting motifs. In some embodiments, one or both of these Lysines in the costimulatory domain of OX40 are mutated Arginines or another amino acid.

### Linker

In some embodiments, the engineered signaling polypeptide includes a linker between any two adjacent domains. For example, a linker can be between the transmembrane domain and the first co-stimulatory domain. As another example, the ASTR can be an antibody and a linker can be between the heavy chain and the light chain. As another example, a linker can be between the ASTR and the transmembrane domain and a co-stimulatory domain. As another example, a linker can be between the co-stimulatory domain and the intracellular activating domain of the second polypeptide. As another example, the linker can be between the ASTR and the intracellular signaling domain.

The linker peptide may have any of a variety of amino acid sequences. Proteins can be joined by a spacer peptide, generally of a flexible nature, although other chemical linkages are not excluded. A linker can be a peptide of between about 1 and about 100 amino acids in length, or between about 1 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ, (GGS)ₙ, (GGGS)ₙ, and (GGGGS)ₙ where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited to, GGGGSGGGGS (SEQ ID NO:674), GGGGSGGGGSGGGGS (SEQ ID NO:63), GGGGSGGGGSGGGGSGGGGS (SEQ ID NO:372), GGGGSGGGGSGGGGSGGGGSGGGGS (SEQ ID NO:675), GGGGSGGGGSGGGGSGGGGSGGGGSGGGGS (SEQ ID NO:64), GGSSRSS (SEQ ID NO:673), GGGGSGGGSGGGGS (SEQ ID NO:65), GGSG (SEQ ID NO:66), GGSGG (SEQ ID NO:67), GSGSG (SEQ ID NO:68), GSGGG (SEQ ID NO:69), GGGSG (SEQ ID NO:70), GSSSG (SEQ ID NO:71), and the like. The ordinarily skilled artisan will recognize that design of a peptide conjugated to any elements described above can include linkers that are all or partially flexible, such that the linker can include a flexible linker as well as one or more portions that confer less flexible structure.

### Combinations

In some embodiments, a polynucleotide provided by the replication incompetent recombinant retroviral particles has one or more transcriptional units that encode certain combinations of the one or more engineered signaling polypeptides. In some methods and compositions provided herein, modified and in illustrative embodiments genetically modified T cells include the combinations of the one or more engineered signaling polypeptides after transduction of T cells by the replication incompetent recombinant retroviral particles. It will be understood that the reference of a first polypeptide, a second polypeptide, a third polypeptide, etc. is for convenience and elements on a “first polypeptide” and those on a “second polypeptide” means that the elements are on different polypeptides that are referenced as first or second for reference and convention only, typically in further elements or steps to that specific polypeptide.

In one embodiment, one or more GOIs are expressed under a cell specific promoter or a general promoter under the same transcript wherein in the transcript, nucleic acids encoding one or more other GOIs are separated by nucleic acids that encode one or more internal ribosomal entry sites (IREs) or one or more protease cleavage peptides.

In any of the aspects or embodiments herein that include an ASTR, the antigen can be a tumor-associated antigen or a tumor-specific antigen. In some embodiments, the tumor-associated antigen or tumor-specific antigen is Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD19, CD20, CD22, CD23, CD24, CD27, CD30, CD33, CD34, CD37, CD38, CD40, CD44, CD44v6, CD44v7/8, CD45, CD70, CD99, CD117, CD123, CD133, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, c-Met, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), PDL-1, GPC3, GUCY2C, HER1, ICAM-1, IL13Rα2, IL11Rα, Kras, Kras G12D, LlCAM, MET, MUC1, MUC16 ecto, NKG2D, NY-ESO-1, PSCA, or WT-1.

### Additional Sequences

The engineered signaling polypeptides, such as CARs, can further include one or more additional polypeptide domains, where such domains include, but are not limited to, a signal sequence; an epitope tag; an affinity domain; and a polypeptide whose presence or activity can be detected (detectable marker), for example by an antibody assay or because it is a polypeptide that produces a detectable signal. Non-limiting examples of additional domains for any of the aspects or embodiments provided herein, include a domain with at least 50%, 60%, 70%, 75%, 80%, 85%, 90%, 95%, 96%, 97%, 98%, 99% or 100% sequence identity to any of the following sequences as disclosed below: a signal sequence, an epitope tag, an affinity domain, or a polypeptide that produces a detectable signal. In some embodiments, the domain can have at least 80% sequence identity to any of the signal sequences disclosed below. In some embodiments, the domain can have at least 90% sequence identity to any of the signal sequences disclosed below. In some embodiments, the domain can have at least 95% sequence identity to any of the signal sequences disclosed below. In some embodiments, the domain can have at least 80% sequence identity to any of the epitope tag sequences disclosed below. In some embodiments, the domain can have at least 90% sequence identity to any of the epitope tag sequences disclosed below. In some embodiments, the domain can have at least 95% sequence identity to any of the epitope tag sequences disclosed below. In some embodiments, the domain can have at least 80% sequence identity to any of the affinity domain sequences disclosed below. In some embodiments, the domain can have at least 90% sequence identity to any of the affinity domain sequences disclosed below. In some embodiments, the domain can have at least 95% sequence identity to any of the affinity domain sequences disclosed below. In some embodiments, the domain can have at least 80% sequence identity to any of the polypeptide sequences that produce a detectable signal disclosed below. In some embodiments, the domain can have at least 90% sequence identity to any of the polypeptide sequences that produce a detectable signal disclosed below. In some embodiments, the domain can have at least 95% sequence identity to any of the polypeptide sequences that produce a detectable signal disclosed below.

Signal sequences that are suitable for use in a subject CAR, e.g., in the first polypeptide of a subject CAR, include any eukaryotic signal sequence, including a naturally-occurring signal sequence, a synthetic (e.g., man-made) signal sequence, etc. In some embodiments, for example, the signal sequence can be the CD8 signal sequence MALPVTALLLPLALLLHAARP (SEQ ID NO:72).

Suitable epitope tags include, but are not limited to, hemagglutinin (HA; e.g., YPYDVPDYA; SEQ ID NO:73); FLAG (e.g., DYKDDDDK; SEQ ID NO:74); c-myc (e.g., EQKLISEEDL; SEQ ID NO:75), and the like.

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), hemagglutinin, e.g., HA Tag (YPYDVPDYA; SEQ ID NO:73), GST, thioredoxin, cellulose binding domain, RYIRS (SEQ ID NO:79), Phe-His-His-Thr (SEQ ID NO:80), chitin binding domain, S-peptide, T7 peptide, SH2 domain, C-end RNA tag, WEAAAREACCRECCARA (SEQ ID NO:81), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCfPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Safety Switch (Recognition and/or Elimination Domain)

Safety switches have been developed for use with cellular therapies to affect the reduction or elimination of infused cells in the case of adverse events. Any of the recombinant viral vectors (e.g., RIPs) provided herein, including those that comprise, for example, membrane-bound cytokines, can include nucleic acids that encode a safety switch as part of, or separate from, nucleic acids encoding any of the engineered signaling polypeptides provided herein. Thus, any of the RIPs that are delivered directly to a subject can comprise nucleic acids that encode safety switches, including for example, anti-idiotype safety switches. Any of the engineered signaling polypeptides provided herein, for example engineered signaling polypeptides in modified, genetically modified, and/or transduced lymphocytes to be introduced or reintroduced into a subject, can include a safety switch. Thus, any of the engineered T cells disclosed herein can include a safety switch.

Safety switch technologies can be broadly categorized into three groups based on their mechanism of action, antibody- or antibody mimetic-mediated cytotoxicity, pro-apoptotic signaling, and metabolic (gene-directed enzyme prodrug therapy, GDEPT). Previously disclosed safety switches include cell surface molecules that are truncated tyrosine kinase receptors. In some of these examples, the truncated tyrosine kinase receptor is a member of the epidermal growth factor receptor (EGFR) family (e.g., ErbB1 (HER1), ErbB2, ErbB3, and ErbB4), for example as disclosed in U.S. Pat. No. 8,802,374 or WO2018226897. Thus, some of these prior safety switches were polypeptides that are recognized by an antibody that recognizes the extracellular domain of an EGFR member. For example, SEQ ID NO:82, is an exemplary polypeptide that is recognized by, and under the appropriate conditions bound by an antibody that recognizes the extracellular domain of an EGFR member. Such truncated EGFR polypeptides are sometimes referred to herein as eTags. In illustrative embodiments, eTags are recognized by monoclonal antibodies that are commercially available such as matuzumab, necitumumab panitumumab, and in illustrative embodiments, cetuximab. For example, eTag was demonstrated to have a cell killing potential through Erbitux® mediated antibody dependent cellular cytotoxicity (ADCC) pathways. The inventors of the present disclosure have successfully expressed eTag in PBMCs using lentiviral vectors, and have found that expression of eTag in vitro by PBMCs exposed to Cetuximab, provided an effective elimination mechanism for PBMCs. eTags can be used in some embodiments of the present disclosure, but in such embodiments, typically an anti-idiotype extracellular domain is present as well.

In some embodiments, the extracellular recognition domain (i.e., cell tag) is itself an antibody, which as disclosed herein includes a functional antibody fragment, that binds a predetermined binding partner antibody (e.g., a target antibody). In illustrative embodiments, the cell tag antibody is specific for the target antibody, and for example, does not bind antibody constant regions exclusively, or in some embodiments, at all, or in illustrative embodiments, unless they interact with the target antibody (Ab1) to cell tag (extracellular recognition domain) (Ab2) binding. In illustrative embodiments, the cell tag antibody (i.e., extracellular recognition domain that includes the variable region of an antibody) is an anti-idiotypic antibody or antibody mimetic. In some embodiments, the anti-idiotypic antibody (Ab2) recognizes an epitope on the predetermined binding partner antibody (i.e., target antibody) (Ab1) that is distinct from the antigen binding site on Ab1. In illustrative embodiments, Ab2 binds the variable region of Ab1. In other illustrative embodiments, Ab2 binds the antigen-binding site of Ab1, and, in illustrative embodiments, competes with Ab1 for binding to the antigen-binding site of Ab1. In certain embodiments, Ab2 may be from any animal including human and murine, or humanized or a chimeric antibody or an antibody derivative included within the definition of “antibody” herein, including, for example antibody fragments (Fab, Fab′, F(ab′)2, scFv, diabodies, bispecific antibodies, and antibody fusion proteins. Ab2 is typically associated with a membrane through a membrane association domain. In certain embodiments, Ab2 is associated with the cell surface via its endogenous transmembrane domain. In other embodiments, Ab2 is associated with the cell surface via a heterologous transmembrane domain or membrane attachment sequence such as GPI. In some embodiments, Ab1 is a commercially available monoclonal antibody. In illustrative embodiments, Ab1 is a commercially available monoclonal antibody therapeutic. In further illustrative embodiments, Ab1 is capable of mediating ADCC and/or CDC as described below. An example of a binding pair comprising an anti-idiotypic antibody (and methods of making the same) displayed on a cell line and cognate monoclonal Ab2 antibodies that mediate ADCC and CDC, is provided in WO2013188864.

In some embodiments, safety switches can also function as flags that label or mark polynucleotides, polypeptides, or cells as being engineered. Such safety switches can be detected using standard laboratory techniques including PCR, Southern Blots, RT-PCR, Northern Blots, Western Blots, histology, and flow cytometry. For example, detection of eTAG by flow cytometry has been used by at least one of the inventors as an in vivo tracking marker for T cell engraftment in mice. In other embodiments, cell tags are used to enrich for engineered cells using antibodies or ligands optionally bound to a solid substrate such as a column or beads. For example, others have shown that application of biotinylated-cetuximab to immunomagnetic selection in combination with anti-biotin microbeads successfully enriches T cells that have been lentivirally transduced with eTAG containing constructs from as low as 2% of the population to greater than 90% purity without observable toxicity to the cell preparation.

In some embodiments provided herein, the anti-idiotype polypeptide is a safety switch (also called a safety switch polypeptide or an anti-idiotype polypeptide safety switch herein) comprising a recognition domain of an anti-idiotype antibody or anti-idiotype antibody mimetic and a membrane association domain. Such safety switch polypeptides can be designed much more efficiently and with many more optional sequences and designs, than prior art safety switches. Such a safety switch polypeptide in one aspect, is designed such that the extracellular recognition domain recognizes an idiotype of an antibody or antibody mimetic capable of inducing cytotoxicity.

Thus, in one aspect the safety switch is based on antibody mediated cytotoxicity upon antibody or antibody mimetic binding to an anti-idiotype polypeptide expressed on the surface of a cell, and more specifically binding to an extracellular recognition domain (also referred to herein as a cell tag or more specifically, an anti-idiotype cell tag) of an anti-idiotype polypeptide. In some embodiments, the antibody or antibody mimetic binds to the cell tag and induces complement-dependent cytotoxicity (CDC) and/or antibody-dependent cell-mediated cytotoxicity (ADCC). In some embodiments, binding of the antibody or antibody mimetic to the anti-idiotype polypeptide induces, promotes, and/or activates one or more of ADCC, CDC, antibody-mediated complement activation, antibody-dependent cellular phagocytosis, and antibody-dependent enhancement of diseases. Details related to other antibody and antibody mimetic functions, including corresponding Fc domains for eliciting such responses, are discussed in the “Antibody and antibody mimetic effector functions” herein. The anti-idiotype polypeptide can be immunogenic, to further stimulate the immune system. Thus, in some embodiments, the cell tag is immunogenic. In other embodiments, the cell tag polypeptide is non-immunogenic. In another aspect, a safety switch polypeptide is designed such that the anti-idiotype polypeptide includes an intracellular domain having one or more cell-death inducing signals, and the polypeptide is capable of inducing a cell death signal upon binding of the anti-idiotype polypeptide to a target antibody or antibody mimetic that comprises the idiotype recognized by the anti-idiotype polypeptide. The cell-death inducing signals can be induced based on dimerization-induced apoptotic signaling. In some embodiments, the safety switch is based on dimerization induced apoptotic signals. In some embodiments, such a safety switch comprises an extracellular dimerization domain comprising a recognition domain of an anti-idiotype antibody or antibody mimetic linked in frame with a membrane association domain and an intracellular domain comprising components of an apoptotic pathway. Thus, dimerization mediated by the binding of an antibody or antibody mimetic to the anti-idiotype polypeptide results in apoptosis of the cell. In some embodiments, the safety switch includes inducible FAS (iFAS) comprised of one or more inducible dimerization domains, i.e., the anti-idiotype polypeptides, fused to the cytoplasmic tail of the Fas receptor and localized to the membrane by a membrane association domain. As discussed in the “Intracellular domains” section herein, in some embodiments, the safety switch includes one or more domains from a Caspase, such as caspase-1 or caspase-9.

The anti-idiotype polypeptides, including the safety switches discussed in this section, can be expressed as fusions with other polypeptides disclosed herein, including a lymphoproliferative element, a CAR, and/or a recombinant TCR. In other embodiments, the anti-idiotype polypeptides are expressed as polypeptides by themselves. In any of these embodiments, the anti-idiotype polypeptides can include any of the domains disclosed herein to be included in a lymphoproliferative element, CAR, and/or TCR, such as the extracellular domains, stalks, transmembrane domains, intracellular activating domains, modulatory domains, linkers, or intracellular domains.

In one aspect the safety switch is based on dimerization induced apoptotic signals. In some embodiments, the safety switch is a chimeric protein comprised of an inducible dimerization domain linked in frame with components of an apoptotic pathway, such that conditional dimerization mediated by the binding of a cell-permeable chemical inducer of dimerization (CID) results in apoptosis of the cell. In some embodiments, the safety switch is inducible FAS (iFAS) comprised of one or more inducible dimerization domains fused to the cytoplasmic tail of the Fas receptor and localized to the membrane by a myristoyl group. In some embodiments, the safety switch is an inducible Caspase comprised of one or more inducible dimerization domains fused to a caspase, such as caspase-1 or caspase-9. In some embodiments the inducible dimerization domain is a cyclophilin and the CID is cyclosporin or a cyclosporin derivative. In some embodiments the inducible dimerization domain is a FKBP and the CID is an FK-506 dimer or derivative thereof, such as AP1903.

In some embodiments, the cell tag is a myc or FLAG tag. In preferred embodiments, the cell tag polypeptide is non-immunogenic. In some embodiments, the modified endogenous cell-surface molecule is a truncated version of a member of the TNF receptor superfamily. For example, a truncated version of the low affinity nerve growth factor receptor (LNGFR or TNFRSF16). Human LNGFR is a single pass type I transmembrane glycoprotein with the amino acid sequence of (SEQ ID NO:369) that comprises a 28 aa residue signal peptide, a 222 aa extracellular domain comprising 4 cysteine rich domains, a 22 aa transmembrane domain and a 155aa intracellular domain. In some embodiments the cell-surface molecule comprises an epitope has at least 70%, 75%, 80%, 85%, 90%, 95%, 99%, or 100% identify to the amino acid sequence of the entire extracellular domain of LNGFR or to a truncated fragment of the extracellular domain such as residues 29-250, 65-250, or 108-250 of SEQ ID NO:369.

In some embodiments, the modified endogenous cell-surface molecule is a version of CD20. The human CD20 polypeptide is a multi-pass transmembrane protein encoded by a membrane-spanning 4-domains subfamily A member (MS4A1) gene with the amino acid sequence of SEQ ID NO:370. In some embodiments, CD20 comprises 4 transmembrane domain passes encompassing amino acids 57-78, 85-105, 121-141, and 189-209. In some embodiments, CD20 comprises 2 extracellular domains encompassing amino acids 79-84 and 142-188. In some embodiments, CD20 comprises 3 cytoplasmic domains encompassing amino acids 1-56, 106-120 and 210-297. In some embodiments, a CD20 polypeptide can be missing multiple domains or multiple portions of a domain relative to the wildtype polypeptide. In an embodiment, a CD20 polypeptide comprises M1-E263, M117-N214, M1-N214, V82-N214, or V82-I186 of endogenous CD20. In an embodiment, a CD20 polypeptide has at least 70%, 75%, 80%, 85%, 90%, 95%, 99%, or 100% identity to an amino acid sequence selected from K142-S185, P160-S185, or C167-C183 of SEQ ID NO:370. In illustrative embodiments, the truncated CD20 version comprises at least one copy of an epitope recognized by a monoclonal antibody such as ocrelizumab, obinutuzumab, ofatumumab, ibritumomab tiuxetan, tositumomab, ublituximab, and in further illustrative embodiments rituximab.

In some embodiments, the modified endogenous cell-surface molecule is a version of CD52. CD52 occurs endogenously in humans as a peptide of 12 amino acids linked at its C-terminus to a GPI anchor. In some embodiments, GPI can be used to anchor the polypeptide to the cell surface. In other embodiments, CD52 can be attached to the cell surface using a heterologous transmembrane domain. In some embodiments, the truncated CD52 polypeptide can incorporate one or more epitopes recognized by an antibody such as HI186 (BioRad), YTH34.5 (BioRad), YTH66.9 (BioRad), or in illustrative embodiments, alemtuzumab. In some embodiments, the CD52 epitope has at least 70%, 75%, 80%, 85%, 90%, 95%, 99%, or 100% identify to the amino acid sequence of SEQ ID NO:371.

In some embodiments, safety switches also function as flags that label or mark polynucleotides, polypeptides, or cells as being engineered. Such safety switches can be detected using standard laboratory techniques including PCR, Southern Blots, RT-PCR, Northern Blots, Western Blots, histology, and flow cytometry. For example, detection of eTAG by flow cytometry was used herein as an in vivo tracking marker for T cell engraftment in mice. In other embodiments, cell tags are used to enrich for engineered cells using antibodies or ligands optionally bound to a solid substrate such as a column or beads. For example, others have shown that application of biotinylated-cetuximab to immunomagnetic selection in combination with anti-biotin microbeads successfully enriches T cells that have been lentivirally transduced with eTAG containing constructs from as low as 2% of the population to greater than 90% purity without observable toxicity to the cell preparation.

In some embodiments, the safety switch is expressed as part of a single polynucleotide that also includes the CAR, or as part of a single polynucleotide that includes the lymphoproliferative element, or as a single polynucleotide that encodes both the CAR and the lymphoproliferative element. In some embodiments the polynucleotide encoding the safety switch is separated from the polynucleotide encoding the CAR and/or the polynucleotide encoding the lymphoproliferative element, by an internal ribosome entry site (IRES) or a ribosomal skip sequence and/or cleavage signal. The ribosomal skip and/or cleavage signal can be any ribosomal skip sequence and/or cleavage signal known in the art. The ribosomal skip sequence can be, for example, T2A with amino acid sequence GSGEGRGSLLTCGDVEENPGP (SEQ ID NO:83). Other examples of cleavage signals and ribosomal skip sequences include FMDV 2A (F2A); equine rhinitis A virus 2A (abbreviated as E2A); porcine teschovirus-1 2A (P2A); and Thoseaasigna virus 2A (T2A).

In some embodiments a safety switch, and in illustrative embodiments, a cell tag, is expressed as part of a fusion polypeptide, fused to a CAR. In other embodiments, a safety switch, and as exemplified empirically herein, a cell tag, is expressed fused to a lymphoproliferative element. Such constructs provide the advantage, especially in combination with other “space saving” elements provided herein, of taking up less genomic space on an RNA genome compared to separate polypeptides. In one illustrative embodiment, an eTag is expressed as a fusion polypeptide, fused the 5′ terminus of the c-Jun domain (SEQ ID NO: 104), a transmembrane domain from CSF2RA (SEQ ID NO: 129), a first intracellular domain from MPL (SEQ ID NO:283), and a second intracellular domain from CD40 (SEQ ID NO:208). When expressed as a polypeptide not fused to a CAR or lymphoproliferative element, the cell tag may be associated with the cell membrane via its natural membrane attachment sequence or via a heterologous membrane attachment sequence such as a GPI-anchor or transmembrane sequence. In illustrative embodiments cell tags are expressed on the T cell and/or NK cell but are not expressed on the replication incompetent recombinant retroviral particles. In some embodiments, polynucleotides, polypeptides, and cells comprise 2 or more safety switches.

### Chimeric Antigen Receptors

In some aspects of the present invention, an engineered signaling polypeptide is a chimeric antigen receptor (CAR) or a polynucleotide encoding a CAR, which, for simplicity, is referred to herein as “CAR.” A CAR of the present disclosure includes: a) at least one antigen-specific targeting region (ASTR); b) a transmembrane domain; and c) an intracellular activating domain. In illustrative embodiments, the antigen-specific targeting region of the CAR is an scFv portion of an antibody to the target antigen. In illustrative embodiments, the intracellular activating domain is from CD3Z, CD3D, CD3E, CD3G, CD79A, CD79B, DAP12, FCER1G, FCGR2A, FCGR2C, DAP10/CD28, or ZAP70, and some further illustrative embodiments, from CD3z. In illustrative embodiments, the CAR further comprises a co-stimulatory domain, for example any of the co-stimulatory domains provided above in the Modulatory Domains section, and in further illustrative embodiments the co-stimulatory domain is the intracellular co-stimulatory domain of 4-1BB (CD137), CD28, ICOS, OX-40, BTLA, CD27, CD30, GITR, and HVEM. In some embodiments, the CAR includes any of the transmembrane domains listed in the Transmembrane Domain section above. In some embodiments, any of the RIPs (RIP formulation and/or a delivery solution) that are delivered directly to a subject can comprise nucleic acids that encode a CAR as disclosed in this section, and in any embodiments herein.

A CAR of the present disclosure can be present in the plasma membrane of a eukaryotic cell, e.g., a mammalian cell, where suitable mammalian cells include, but are not limited to, a cytotoxic cell, a T lymphocyte, a stem cell, a progeny of a stem cell, a progenitor cell, a progeny of a progenitor cell, and an NK cell, an NK-T cell, and a macrophage. When present in the plasma membrane of a eukaryotic cell, a CAR of the present disclosure is active in the presence of one or more target antigens that, in certain conditions, binds the ASTR. The target antigen is the second member of the specific binding pair. The target antigen of the specific binding pair can be a soluble (e.g., not bound to a cell) factor; a factor present on the surface of a cell such as a target cell; a factor presented on a solid surface; a factor present in a lipid bilayer; and the like. Where the ASTR is an antibody, and the second member of the specific binding pair is an antigen, the antigen can be a soluble (e.g., not bound to a cell) antigen; an antigen present on the surface of a cell such as a target cell; an antigen presented on a solid surface; an antigen present in a lipid bilayer; and the like.

In some embodiments, the ASTR of a CAR is expressed as a separate polypeptide from the intracellular signaling domain. In such embodiments, one or both of the polypeptides can include any of the transmembrane domains disclosed herein. In some embodiments, one or both of the polypeptides can include a heterologous signal sequence and/or a heterologous membrane attachment sequence. In some embodiments, the heterologous membrane attachment sequence is a GPI anchor attachment sequence.

In some instances, a CAR of the present disclosure, when present in the plasma membrane of a eukaryotic cell, and when activated by one or more target antigens, increases expression of at least one nucleic acid in the cell. For example, in some cases, a CAR of the present disclosure, when present in the plasma membrane of a eukaryotic cell, and when activated by the one or more target antigens, increases expression of at least one nucleic acid in the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the level of transcription of the nucleic acid in the absence of the one or more target antigens.

As an example, the CAR of the present disclosure can include an immunoreceptor tyrosine-based activation motif (ITAM)-containing intracellular signaling polypeptide.

A CAR of the present disclosure, when present in the plasma membrane of a eukaryotic cell, and when activated by one or more target antigens, can, in some instances, result in increased production of one or more cytokines by the cell. For example, a CAR of the present disclosure, when present in the plasma membrane of a eukaryotic cell, and when activated by the one or more target antigens, can increase production of a cytokine by the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the cell in the absence of the one or more target antigens. Cytokines whose production can be increased include, but are not limited to, interferon gamma (IFN-γ), tumor necrosis factor-alpha (TNF-α), IL-2, IL-15, IL-12, IL-4, IL-5, IL-10; a chemokine; a growth factor; and the like.

In some embodiments, a CAR of the present disclosure, when present in the plasma membrane of a eukaryotic cell, and when activated by one or more target antigens, can result in both an increase in transcription of a nucleic acid in the cell and an increase in production of a cytokine by the cell.

In some instances, a CAR of the present disclosure, when present in the plasma membrane of a eukaryotic cell, and when activated by one or more target antigens, results in cytotoxic activity by the cell toward a target cell that expresses on its cell surface an antigen to which the antigen-binding domain of the first polypeptide of the CAR binds. For example, where the eukaryotic cell is a cytotoxic cell (e.g., an NK cell or a cytotoxic T lymphocyte), a CAR of the present disclosure, when present in the plasma membrane of the cell, and when activated by the one or more target antigens, increases cytotoxic activity of the cell toward a target cell that expresses on its cell surface the one or more target antigens. For example, where the eukaryotic cell is an NK cell or a T lymphocyte, a CAR of the present disclosure, when present in the plasma membrane of the cell, and when activated by the one or more target antigens, increases cytotoxic activity of the cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the cell in the absence of the one or more target antigens.

In some embodiments, a CAR of the present disclosure, when present in the plasma membrane of a eukaryotic cell, and when activated by one or more target antigens, can result in other CAR activation related events such as proliferation and expansion (either due to increased cellular division or anti-apoptotic responses).

In some embodiments, a CAR of the present disclosure, when present in the plasma membrane of a eukaryotic cell, and when activated by one or more target antigens, can result in other CAR activation related events such as intracellular signaling modulation, cellular differentiation, or cell death.

In some embodiments, CARs of the present disclosure are microenvironment restricted. This property is typically the result of the microenvironment restricted nature of the ASTR domain of the CAR. Thus, CARs of the present disclosure can have a lower binding affinity or, in illustrative embodiments, can have a higher binding affinity to one or more target antigens under a condition(s) in a microenvironment than under a condition in a normal physiological environment.

In certain illustrative embodiments, CARs provided herein comprise a co-stimulatory domain in addition to an intracellular activating domain, wherein the co-stimulatory domain is any of the intracellular signaling domains provided herein for lymphoproliferative elements (LEs), such as, for example, intracellular domains of CLEs. In certain illustrative embodiments, the co-stimulatory domains of CARs herein are first intracellular domains (P3 domains) identified herein for CLEs or P4 domains that are shown as effective intracellular signaling domains of CLEs herein in the absence of a P3 domain. Furthermore, in certain illustrative embodiments, co-stimulatory domains of CARs can comprise both a P3 and a P4 intracellular signaling domain identified herein for CLEs. Certain illustrative subembodiments include especially effective P3 and P4 partner intracellular signaling domains as identified herein for CLEs. In illustrative embodiments, the co-stimulatory domain is other than an ITAM-containing intracellular domain of a CAR either as part of the co-stimulatory domain, or in further illustrative embodiments as the only co-stimulatory domain.

In these embodiments that include a CAR with a co-stimulatory domain identified herein as an effective intracellular domain of an LE, the co-stimulatory domain of a CAR can be any intracellular signaling domain in Table 1 provided herein. Active fragments of any of the intracellular domains in Table 1 can be a co-stimulatory domain of a CAR. In illustrative embodiments, the ASTR of the CAR comprises an scFV. In illustrative embodiments, in addition to the c-stimulatory intracellular domain of a CLE, these CARs comprise an intracellular activating domain that in illustrative embodiments is a CD3Z, CD3D, CD3E, CD3G, CD79A, CD79B, DAP12, FCER1G, FCGR2A, FCGR2C. DAP10/CD28, or ZAP70 intracellular activating domain, or in further illustrative embodiments is a CD3z intracellular activating domain.

In these illustrative embodiments, the co-stimulatory domain of a CAR can comprise an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL7RA, IL9R, IL10RA, IL10RB, IL11RA, IL12RB1, IL12RB2, IL13RA1, IL13RA2, IL15RA, IL17RB, IL17RC, IL17RD, IL18R1, IL18RAP, IL20RA, IL20RB, IL21R, IL22RA1, IL23R, IL27RA, IL31RA, LEPR, LIFR, LMP1, MPL, MyD88, OSMR, or PRLR. In some embodiments, the co-stimulatory domain of a CAR can include an intracellular domain or a functional signaling fragment thereof that includes a signaling domain from CSF2RB, CRLF2, CSF2RA, CSF3R, EPOR, GHR, IFNAR1, IFNAR2, IFNGR1, IFNGR2, IFNLR1, IL1R1, IL1RAP, IL1RL1, IL1RL2, IL2RA, IL2RB, IL2RG, IL3RA, IL5RA, IL6R, IL6ST, IL9R, IL10RA, IL10RB, IL11RA, IL13RA1, IL13RA2, IL17RB, IL17RC, IL17RD, IL18R1, IL18RAP, IL20RA, IL20RB, IL22RA1, IL31RA, LEPR, LIFR, LMP1, MPL, MyD88, OSMR, or PRLR. In some embodiments, the co-stimulatory domain of a CAR can include an intracellular domain or a functional fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL12RB1, IL12RB2, IL13RA2, IL15RA, IL17RD, IL21R, IL23R, IL27RA, IL31RA, LEPR, MPL, MyD88, or OSMR. In some embodiments, the co-stimulatory domain of a CAR can include an intracellular domain or a fragment thereof that includes a signaling domain from CSF2RB, CSF2RA, CSF3R, EPOR, IFNGR1, IFNGR2, IL1R1, IL1RAP, IL1RL1, IL2RA, IL2RG, IL5RA, IL6R, IL9R, IL10RB, IL11RA, IL13RA2, IL17RD, IL31RA, LEPR, MPL, MyD88, or OSMR. In some embodiments, the co-stimulatory domain of a CAR can include an intracellular domain or a functional signaling fragment thereof that includes a signaling domain from CSF2RB, CSF3R, IFNAR1, IFNGR1, IL2RB, IL2RG, IL6ST, IL10RA, IL12RB2, IL17RC, IL17RE, IL18R1, IL27RA, IL31RA, MPL, MyD88, OSMR, or PRLR. In some embodiments, the co-stimulatory domain of a CAR can include an intracellular domain or a functional signaling fragment thereof that includes a signaling domain from CSF2RB, CSF3R, IFNGR1, IL2RB, IL2RG, IL6ST, IL10RA, IL17RE, IL31RA, MPL, or MyD88.

In some embodiments, the co-stimulatory domain of a CAR can include an intracellular domain or a fragment thereof that includes a signaling domain from CSF3R, IL6ST, IL27RA, MPL, and MyD88. In certain illustrative subembodiments, the intracellular activating domain of the CAR is derived from CD3z.

The present disclosure in some aspects provides methods of administering, which in illustrative embodiments are treatment methods using a CAR for cancers that are heterogeneous. Without being bound by theory, cancer heterogeneity refer to the existence of subpopulations of cells, with distinct genotypes and phenotypes that may harbor divergent biological behaviors, within a primary tumor and its metastases. Cancers with a high level of heterogeneity typically predispose subjects to inferior clinical outcomes. Such heterogeneous cancers may vary in the expression on the surface of the cancer cells of the antigen to which a CAR binds. In such cancers, a CAR cell that only kills cells expressing the antigen would be unable to bind and kill the cancer cells not expressing the antigen. However, some CAR cells are able to kill neighboring cells as part of the binding and killing of a target cell. This process is also known as the bystander effect. In some embodiments, a CAR of the present disclosure binds to an antigen present on a target cell, thereby mediating killing of the target cell by the genetically modified lymphocyte expressing the CAR, wherein one or more neighbor cells are killed and/or the growth of one or more neighbor cells is inhibited. In some embodiments, neighbor cells can be with 1, 2, 3, 4, 5, 6, 7, 8, 9, 10, 11, 12, 13, 14, 15, 16, 17, 18, 19, or 20 cells of the target cell. In some embodiments, the neighbor cells are within 100, 200, 300, 400, 500, 600, 700, 800, or 900 m or 1, 2, 3, 4, 5, 6, 7, 8, 9, or 10 mm of a target cell. In some embodiments, the neighbor cells comprise cancer cells. In some embodiments, the neighbor cells comprise cells that do not express the antigen to which the CAR binds. In illustrative embodiments, the neighbor cells comprise cancer cells that do not express the antigen to which the CAR binds. In some embodiments, a cancer is a heterogeneous cancer for expression of an antigen to which the CAR binds if at least 5%, 10%, 15%, 20%, 25%, 30%, 35%, 40%, 45%, 50%, 55%, 60%, 65%, 70%, 75%, 80%, 85%, 90%, or 95% of the cells in the cancer do not express the antigen on the surface of the cells. In some embodiments, a cancer is a heterogeneous cancer for expression of an antigen to which the CAR binds if at least 5%, 10%, 15%, 20%, 25%, 30%, 35%, 40%, 45%, 50%, 55%, 60%, 65%, 70%, 75%, 80%, 85%, 90%, or 95% of the cells in the cancer express the antigen at a level on the surface of the cells that is insufficient for binding and killing by a CAR that binds the antigen. In certain illustrative embodiments of any of the aspects that include administering to a subject with a heterogeneous cancer, the cells or cell population administered comprise CAR cells that exhibit a T and NK cell-like phenotype. In some embodiments, the CD3+ CAR cells express NKG2D, CD56, and/or CD57. In illustrative embodiments, the CD3+ CAR cells express NKG2D, CD56, and/or CD57 in addition to CD8. Without being bound by theory, the killing of target cells by CD56+ and/or CD57+ cells that are either CD8+ or CD4+ cells genetically modified to express a CAR is more effective due to the bystander effects exhibited by these cells. Owing to the bystander effects, the CD56+ and/or CD57+ cells that are either CD8+ or CD4+ cells modified to express a CAR also induce killing of cancer cells that are not expressing the antigen. In some embodiments, CD56+ and/or CD57+ cells that are either CD8+ or CD4+ cells modified to express a CAR as disclosed herein are effective in killing cancer cells that express antigens heterogeneously and at different levels.

Another method to reduce the potential effects of CAR-cancer cells is to use two or more separate CARs, and in illustrative embodiments, two CARs expressed in two populations of cells, to kill target cells that could mask one of the epitopes. A population of cells, such as blood cells or PBMCs, are genetically modified separately so each population expresses either a first CAR or a second CAR. In illustrative embodiments, a target cell expressing the first or second CAR does not mask the epitope that the second and first CAR, respectively, bind to. Therefore, a target cell expressing the first or second CAR can be killed by an effector T or NK cell expressing the second or first CAR, respectively. In some embodiments, the first and second CARs can bind to different epitopes of the same antigen expressed on a target cell. In other embodiments, the first and second CARs can bind to different antigens expressed on the same target cell, including any of the antigens disclosed elsewhere herein. In some embodiments, the first and second CARs can bind to different epitopes of, or different antigens selected from CD19, CD20, CD22, CD25, CD32, CD34, CD38, CD123, BCMA, TACI or TIM3. In further illustrative embodiments, the first CAR can bind to CD19 and the second CAR can bind to CD22, both of which are expressed on B cells. In other embodiments, the CAR can be an extracellular ligand of a cancer antigen. In illustrative embodiments, the modified cell populations are formulated separately. In some embodiments, the separate cell formulations are introduced or reintroduced back into the subject at different sites in the body. In some embodiments, separate cell formulations are separately introduced or reintroduced back into the subject at the same site. In other embodiments, the modified cell populations are combined into one formulation that is optionally introduced or reintroduced back into the subject together at the same site. In illustrative embodiments wherein the cell populations are combined, the cell populations are not combined until after a washing step in which the cells are washed away from the recombinant nucleic acid vectors. By this method of using two or more distinct CARs, a CAR-cancer cell expressing a first or second CAR that binds and masks its cognate epitope in cis, will be killed by a CAR-T cell expressing the second or first CAR, respectively.

### Recombinant T Cell Receptors (TCRs)

T Cell Receptors (TCRs) recognize specific protein fragments derived from intracellular as well as extracellular proteins. When proteins are broken into peptide fragments, they are presented on the cell surface with another protein called major histocompatibility complex, or MHC, which is called the HLA (human leukocyte antigen) complex in humans. Three different T cell antigen receptors combinations in vertebrates are αβ TCR, γδTCR and pre-TCR. Such combinations are formed by dimerization between members of dimerizing subtypes, such as an a TCR subunit and a 0 TCR subunit, a γ TCR subunit and a 6 TCR subunit, and for pre-TCRs, a pTα subunit and a 0 TCR subunit. A set of TCR subunits dimerize and recognize a target peptide fragment presented in the context of an MHC. The pre-TCR is expressed only on the surface of immature αβ T cells while the αβ TCR is expressed on the surface of mature αβ T cells and NK T cells, and γδTCR is expressed on the surface of γδT cells. αβTCRs on the surface of a T cell recognize the peptide presented by MHCI or MHCII and the αβ TCR on the surface of NK T cells recognize lipid antigens presented by CD1. γδTCRs can recognize MHC and MHC-like molecules, and can also recognize non-MHC molecules such as viral glycoproteins. Upon ligand recognition, αβTCRs and γδTCRs transmit activation signals through the CD3zeta chain that stimulate T cell proliferation and cytokine secretion.

TCR molecules belong to the immunoglobulin superfamily with its antigen-specific presence in the V region, where CDR3 has more variability than CDR1 and CDR2, directly determining the antigen binding specificity of the TCR. When the MHC-antigen peptide complex is recognized by a TCR, the CDR1 and CDR2 recognize and bind the sidewall of the MHC molecule antigen binding channel, and the CDR3 binds directly to the antigenic peptide. Recombinant TCRs may thus be engineered that recognize a tumor-specific protein fragment presented on MHC.

Recombinant TCR's such as those derived from human TCRα and TCRβ pairs that recognize specific peptides with common HLAs can thus be generated with specificity to a tumor specific protein (Schmitt, T M et al., 2009). The target of recombinant TCRs may be peptides derived from any of the antigen targets for CAR ASTRs provided herein, but are more commonly derived from intracellular tumor specific proteins such as oncofetal antigens, or mutated variants of normal intracellular proteins or other cancer specific neoepitopes. Libraries of TCR subunits may be screened for their selectivity to a target antigen. Screens of natural and/or recombinant TCR subunits can identify sets of TCR subunits with high avidities and/or reactivities towards a target antigen. Members of such sets of TCR subunits can be selected and cloned to produce one or more polynucleotide encoding the TCR subunit.

Polynucleotides encoding such a set of TCR subunits can be included in a replication incompetent recombinant retroviral particle to genetically modify a lymphocyte, or in illustrative embodiments, a T cell or an NK cell, such that the lymphocyte expresses the recombinant TCR. In some embodiments, RIP comprising nucleic acids that encode a TCR as disclosed in this section, and in any embodiments herein modifies a lymphocyte, such as, a T cell and/or a NK cell, in vivo. In some embodiments, the RIP comprising nucleic acids that encode TCR modifies a lymphocyte, such as, a T cell and/or a NK cell, ex vivo. Accordingly, in any aspect or embodiment provided herein that includes a polynucleotide encoding a CAR or an engineered signaling polypeptide that is a CAR, the CAR can be replaced by a set of γδTCR chains, or in illustrative embodiments αβTCR chains. TCR chains that form a set may be co-expressed using a number of different techniques to co-express the two TCR chains as is disclosed herein for expressing two or more other engineered signaling polypeptides such as CARs and lymphoproliferative elements. For example, protease cleavage epitopes such as 2A protease, internal ribosomal entry sites (IRES), and separate promoters may be used. In some embodiments, any of the RIP (RIP formulation and/or a delivery solution) that are delivered directly to a subject can comprise nucleic acids that encode a TCR as disclosed in this section, and in any embodiments herein.

Several strategies have been employed to reduce the likelihood of mixed TCR dimer formation. In general, this involves modification of the constant (C) domains of the TCRα and TCRβ chains to promote the preferential pairing of the introduced TCR chains with each other, while rendering them less likely to successfully pair with endogenous TCR chains. One approach that has shown some promise in vitro involves replacement of the C domain of human TCRα and TCRβ chains with their mouse counterparts. Another approach involves mutation of the human TCRα common domain and TCRβ chain common regions to promote self-pairing, or the expression of an endogenous TCR alpha and TCR beta miRNA within the viral gene construct. Accordingly, in some embodiments provided herein that include one or more sets of TCR chains as engineered signaling polypeptides, each member of the set of TCR chains, in illustrative embodiments αβTCR chains, comprises a modified constant domain that promotes preferential pairing with each other. In some subembodiments, each member of a set of TCR chains, in illustrative embodiments αβTCR chains, comprises a mouse constant domain from the same TCR chain type, or a constant domain from the same TCR chain subtype with enough sequences derived from a mouse constant domain from the same TCR chain subtype, such that dimerization of the set of TCR chains to each other is preferred over, or occurs to the exclusion of, dimerization with human TCR chains. In other subembodiments, each member of a set of TCR chains, in illustrative embodiments αβTCR chains, comprises corresponding mutations in its constant domain, such that dimerization of the set of TCR chains to each other is preferred over, or occurs to the exclusion of, dimerization with TCR chains that have human constant domains. Such preferred or exclusive dimerization in illustrative embodiments, is under physiological conditions.

In some embodiments provided herein that include one or more sets of TCR chains as engineered signaling polypeptides, the constant regions of the members of each of the one or more sets of TCR chains are swapped. Thus, the α TCR subunit of the set has a f TCR constant region, and the β TCR subunit of the set has a α TCR constant region. Not to be limited by theory, it is believed that such swapping may prevent mispairing with endogenous counterparts.

### Lymphoproliferative Elements

Many of the embodiments provided herein include a lymphoproliferative element, or a nucleic acid encoding the same, typically as part of an engineered signaling polypeptide. Accordingly, in some aspects of the present invention, for example for modified and/or genetically modified lymphocytes to be introduced or reintroduced by subcutaneous injection or modifying the lymphocytes in vivo, an engineered signaling polypeptide is a lymphoproliferative element (LE) such as a chimeric lymphoproliferative element (CLE). In some embodiments, any of the RIP (RIP formulation and/or a delivery solution) that are delivered directly to a subject comprises nucleic acid encoding an LE, such as a CLE. In some embodiments, such an in vivo delivery of the RIP provides in vivo modification of lymphocytes. Typically, the LE comprises an extracellular domain, a transmembrane domain, and at least one intracellular signaling domain that drives proliferation, and in illustrative embodiments a second intracellular signaling domain.

The extracellular domains, transmembrane domains, and intracellular domains of LEs can vary in their respective amino acid lengths. For example, for embodiments that include a replication incompetent retroviral particle (RIP), there are limits to the length of a polynucleotide that can be packaged into a retroviral particle so LEs with shorter amino acid sequences can be advantageous in certain illustrative embodiments. In some embodiments, the overall length of the LE can be between 3 and 4000 amino acids, for example between 10 and 3000, 10 and 2000, 50 and 2000, 250 and 2000 amino acids, and, in illustrative embodiments between 50 and 1000, 100 and 1000 or 250 and 1000 amino acids. The extracellular domain, when present to form an extracellular and transmembrane domain, can be between 1 and 1000 amino acids, and is typically between 4 and 400, between 4 and 200, between 4 and 100, between 4 and 50, between 4 and 25, or between 4 and 20 amino acids. In one embodiment, the extracellular region is GGGS for an extracellular and transmembrane domain of this aspect of the invention. The transmembrane domains, or transmembrane regions of extracellular and transmembrane domains, can be between 10 and 250 amino acids, and are more typically at least 15 amino acids in length, and can be, for example, between 15 and 100, 15 and 75, 15 and 50, 15 and 40, or 15 and 30 amino acids in length. The intracellular signaling domains can be, for example, between 10 and 1000, 10 and 750, 10 and 500, 10 and 250, or 10 and 100 amino acids. In illustrative embodiments, the intracellular signaling domain can be at least 30, or between 30 and 500, 30 and 250, 30 and 150, 30 and 100, 50 and 500, 50 and 250, 50 and 150, or 50 and 100 amino acids. In some embodiments, an intracellular signaling domain for a particular gene is at least 50%, 60%, 70%, 75%, 80%, 85%, 90%, 95%, 98%, 99% or 100% identical to at least 10, 25, 30, 40, 50, or all the amino acids from a sequence of that intracellular signaling domain, such as a sequence provided herein for that intracellular domain, up to the size of the entire intracellular domain sequence, and can include for example, up to an additional 1, 2, 3, 4, 5, 10, 20, or 25 amino acids, provided that such sequence still is capable of providing any of the properties of LEs disclosed herein.

In some embodiments, the lymphoproliferative element can include a first and/or second intracellular signaling domain. In some embodiments, the first and/or second intracellular signaling domain can include CD2, CD3D, CD3E, CD3G, CD4, CD8A, CD8B, CD27, mutated Delta Lek CD28, CD28, CD40, CD79A, CD79B, CRLF2, CSF2RB, CSF2RA, CSF3R, EPOR, FCER1G, FCGR2C, FCGRA2, GHR, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, LEPR, LIFR, LMP1, MPL, MYD88, OSMR, PRLR, TNFRSF4, TNFRSF8, TNFRSF9, TNFRSF14, or TNFRSF18, or functional mutants and/or fragments thereof. In illustrative embodiments, the first intracellular signaling domain can include MyD88, or a functional mutant and/or fragment thereof. In further illustrative embodiments, the first intracellular signaling domain can include MyD88, or a functional mutant and/or fragment thereof, and the second intracellular signaling domain can include ICOS, TNFRSF4, or TNSFR18, or functional mutants and/or fragments thereof. In some embodiments, the first intracellular domain is MyD88 and the second intracellular domain is an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70. In some embodiments, the second intracellular signaling domain can include TNFRSF18, or a functional mutant and/or fragment thereof.

In some embodiments, the lymphoproliferative element can include a fusion of an extracellular domain and a transmembrane domain. In some embodiments, the fusion of an extracellular domain and a transmembrane domain can include eTAG IL7RA Ins PPCL (interleukin 7 receptor), Myc LMP1, LMP1, eTAG CRLF2, eTAG CSF2RB, eTAG CSF3R, eTAG EPOR, eTAG GHR, eTAG truncated after Fn F523C IL27RA, or eTAG truncated after Fn S505N MPL, or functional mutants and/or fragments thereof. In some embodiments, the lymphoproliferative element can include an extracellular domain. In some embodiments, the extracellular domain can include cell tag with 0, 1, 2, 3, or 4 additional alanines at the carboxy terminus. In some embodiments, the extracellular domain can include Myc or an eTAG with 0, 1, 2, 3, or 4 additional alanines at the carboxy terminus, or functional mutants and/or fragments thereof. For any embodiment of a lymphoproliferative element disclosed herein that includes a cell tag, there is a corresponding embodiment that is identical but lacks the cell tag and optionally lacks any linker sequence that connected the cell tag to the lymphoproliferative element.

In some embodiments, the lymphoproliferative element can include a transmembrane domain. In some embodiments, the transmembrane domain can include a transmembrane domain from BAFFR, C3Z, CEACAM1, CD2, CD3A, CD3B, CD3D, CD3E, CD3G, CD3Z, CD4, CD5, CD7, CD8A, CD8B, CD9, CD11A, CD11B, CD11C, CD11D, CD16, CD18, CD19, CD22, CD27, CD28, CD29, CD33, CD37, CD40, CD45, CD49A, CD49D, CD49F, CD64, CD79A, CD79B, CD80, CD84, CD86, CD96 (Tactile), CD100 (SEMA4D), CD103, C134, CD137, CD154, CD160 (BY55), CD162 (SELPLG), CD226 (DNAM1), CD229 (Ly9), CD247, CRLF2, CRTAM, CSF2RA, CSF2RB, CSF3R, EPOR, FCER1G, FCGR2C, FCGRA2, GHR, HVEM (LIGHTR), IA4, ICOS, IFNAR1, IFNAR2, IFNGR1, IFNGR2, IFNLR1, IL1R1, IL1RAP, IL1RL1, IL1RL2, IL2RA, IL2RB, IL2RG, IL3RA, IL4R, IL5RA, IL6R, IL6ST, IL7RA, IL7RA Ins PPCL, IL9R, IL10RA, IL10RB, IL11RA, IL12RB1, IL12RB2, IL13RA1, IL13RA2, IL15RA, IL17RA, IL17RB, IL17RC, IL17RD, IL17RE, IL18R1, IL18RAP, IL20RA, IL20RB, IL21R, IL22RA1, IL23R, IL27RA, IL31RA, ITGA1, ITGA4, ITGA6, ITGAD, ITGAE, ITGAL, ITGAM, ITGAX, ITGB1, ITGB2, ITGB7, KIRDS2, LEPR, LFA-1 (CD11a, CD18), LIFR, LTBR, MPL, NKp80 (KLRF1), OSMR, PAG/Cbp, PRLR, PSGL1, SLAM (SLAMF1, CD150, IPO-3), SLAMF4 (CD244, 2B4), SLAMF6 (NTB-A, Ly108), SLAMF7, SLAMF8 (BLAME), TNFR2, TNFRSF4, TNFRSF8, TNFRSF9, TNFRSF14, TNFRSF18, VLA1, or VLA-6, or functional mutants and/or fragments thereof. Transmembrane™ domains suitable for use in any engineered signaling polypeptide include, but are not limited to, constitutively active cytokine receptors, the TM domain from LMP1, and TM domains from type 1 TM proteins comprising a dimerizing motif, as discussed in more detail herein. In any of the aspects disclosed herein containing the transmembrane domain from a type I transmembrane protein, the transmembrane domain can be a Type I growth factor receptor, a hormone receptor, a T cell receptor, or a TNF-family receptor.

Exemplary extracellular and transmembrane domains for CLEs of embodiments that include such domains, in illustrative embodiments, are extracellular regions, typically less than 30 amino acids of the membrane-proximal extracellular domains along with transmembrane domains from mutant receptors that have been reported to be constitutive, that is not require ligand binding for activation of an associated intracellular domain. In illustrative embodiments, such extracellular and transmembrane domains include IL7RA Ins PPCL, CRLF2 F232C, CSF2RB V449E, CSF3R T640N, EPOR L251C I252C, GHR E260C I270C, IL27RA F523C, and MPL S505N. In some embodiments, the extracellular and transmembrane domain does not comprise more than 10, 20, 25 30 or 50 consecutive amino acids that are identical in sequence to a portion of the extracellular and/or transmembrane domain of IL7RA, or a mutant thereof. In some embodiments, the extracellular and transmembrane domain is other than IL7RA Ins PPCL. In some embodiments, the extracellular and transmembrane does not comprise more than 10, 20, 25, 30, or 50 consecutive amino acids that are identical in sequence to a portion of the extracellular and/or transmembrane domain of IL15R.

In some embodiments, the extracellular and transmembrane domain is the viral protein LMP1, or a mutant and/or fragment thereof. LMP1 is a multispan transmembrane protein that is known to activate cell signaling independent of ligand when targeted to lipid rafts or when fused to CD40 (Kaykas et al. EMBO J. 20: 2641 (2001)). A fragment of LMP1 is typically long enough to span a plasma membrane and to activate a linked intracellular domain(s). For example, the LMP1 can be between 15 and 386, 15 and 200, 15 and 150, 15 and 100, 18 and 50, 18 and 30, 20 and 200, 20 and 150, 20 and 50, 20 and 30, 20 and 100, 20 and 40, or 20 and 25 amino acids. A mutant and/or fragment of LMP1 when included in a CLE provided herein, retains its ability to activate an intracellular domain. Furthermore, if present, the extracellular domain includes at least 1, but typically at least 4 amino acids and is typically linked to another functional polypeptide, such as a clearance domain, for example, an eTag. In some embodiments, the lymphoproliferative element comprises an LMP1 transmembrane domain. In illustrative embodiments, the lymphoproliferative element comprises an LMP1 transmembrane domain and the one or more intracellular domains do not comprise an intracellular domain from TNFRSF proteins (i.e., CD40, 4-IBB, RANK, TACI, OX40, CD27, GITR, LTR, and BAFFR), TLR1 to TLR13, integrins, FcγRIII, Dectin1, Dectin2, NOD1, NOD2, CD16, IL-2R, Type III interferon receptor, chemokine receptors such as CCR5 and CCR7, G-protein coupled receptors, TREM1, CD79A, CD79B, Ig-alpha, IPS-1, MyD88, RIG-1, MDA5, CD3Z, MyD88ATIR, TRIF, TRAM, TIRAP, MAL, BTK, RTK, RAC1, SYK, NALP3 (NLRP3), NALP3ALRR, NALP1, CARD9, DAI, IPAG, STING, Zap70, or LAT.

In other embodiments of CLEs provided herein, the extracellular domain includes a dimerizing moiety. Many different dimerizing moieties disclosed herein can be used for these embodiments. In certain illustrative embodiments, the dimerizing moieties are capable of homodimerizing. Not to be limited by theory, dimerizing moieties can provide an activating function on intracellular domains connected thereto via transmembrane domains.

CLEs for use in any aspect or embodiment herein can include any CLE disclosed in WO2019/055946 (incorporated by reference herein, in its entirety), the vast majority of which were designed to be and are believed to be constitutively active, typically because they constitutively activate a signaling pathway, typically through functional domains on their intracellular domains. In some embodiments, the constitutively active signaling pathways include activation of a Jak pathway, a Stat pathway, or Jak/Stat pathways including Jak1, Jak2, Jak3, and tyk2 and STATs such as STAT1, STAT2, STAT3, STAT4, STAT5, STAT6, and in illustrative embodiments, STAT3 and/or STAT5. Illustrative embodiments of LEs herein include a JAK-binding domain and/or a STAT-recruiting domain. Accordingly, provided herein, in certain embodiments, are lymphoproliferative elements that comprise a means for activating any one or more of these pathways, which typically comprises an intracellular domain that is a means for activating any one or more of these pathways. In certain embodiments, lymphoproliferative elements comprise a means, such as an intracellular domain, that is a means for transmitting a signal that promotes proliferation and/or survival of a T cell and/or NK cell, in illustrative embodiments when part of a dimerized lymphoproliferative element. In some embodiments, a CLE includes one or more Jak binding domains. In some embodiments, a CLE includes one or more Stat recruitment domains. Without being bound to theory or mechanism, in some embodiments, an LE herein that includes a JAK-binding domain and a STAT-recruiting domain dimerizes and/or clusters and allows for two bound JAK-proteins to become activated, which in turn phosphorylate tyrosine residues on a recruiting domain of the LE. The phosphorylated recruiting domains are then capable of binding the recruited proteins (e.g., a phosphorylated STAT-recruiting domain binds a STAT protein), the STAT protein is activated (e.g., by phosphorylation), dissociates from the STAT-recruiting domain, and translocates to the nucleus where the STAT protein affects transcription events.

In some embodiments, a CLE includes one or more STAT-activation domains. In some embodiments, a CLE includes two or more, three or more, four or more, five or more, or six or more STAT-activation domains. In some embodiments, at least one of the one or more STAT-activation domains is, or is derived from BLNK, IL2RG, EGFR, EpoR, GHR, IFNAR1, IFNAR2, IFNAR1/2, IFNLR1, IL10R1, IL12Rb1, IL12Rb2, IL21R, IL2Rb, IL2small, IL7R, IL7Ra, IL9R, IL15R, and IL21R, as are known in the art. In some embodiments, two or more STAT-activation domains are, or are derived from two or more different receptors. Other STAT-activation domains that can be included in aspects and embodiments herein that include an LE, include one or more of IL7R (316-459), IL2Rb (333-551), IFNAR1 (508-557), IFNAR2 (310-515), IFNAR1/2 (IFNAR1 residues 508-557-IFNAR2 residues 310-515), IFNLR1 (300-520), Common Gamma Chain (335-369), IL9R (356-521), IL21R (322-538), GHR (353-638), EpoR (339-508), murine IL2Rb (337-539), murine IL7Ra (316-459), EGFR (955-1186), EGFR (955-1186; Y974F, d1045-1057), EGFR (955-1009; Y974F), EGFR (1019-1085), EGFR (1037-1103; Y1068/1101F, d1045-1057), EGFR (1066-1118; Y1068/1086F), EGFR (1122-1165), EGFR (1133-1186; Y1148F), IL12 Rb2 (775-825), IL7R (376-416), IL7R (424-459), IL7R (376-416, 424-459), IL7R (424-459; Y456F), IL7R (376-416, 424-459, Y456F), IL2Rbsmall (393-433), IL2Rbsmall (518-551), IL2Rbsmall (339-379, 393-433), IL2Rbsmall (339-379, 518-551), IL2Rbsmall (393-433, 518-551), IL2Rbsmall (339-379, 393-433, 518-551), IFNAR2small (310-352), IFNAR2small (486-515), IFNAR2small (310-352, 486-515), BLNK (53-208), BLNK (53-208; Y72F), BLNK (53-208; Y72F, Y96F), EpoR (339-508), IL12Rb2 (714-862), IL12Rb1 (622-662), IL10R1 (304-578), IL2Rb (333-551, Y381S, Y384S, Y387S), and IL2Rb (333-551, Y364S, Y381S, Y384S, Y387S). These STAT-activation domains are provided in SEQ ID NOs:376 to 420, respectively.

In some embodiments, STAT-activation domains, which can also be called STAT-recruiting domains herein, can be linked in tandem to stimulate multiple pathways (e.g., the IL7R(316-459)-IL12Rb2(775-825) fragment fusion for pro-persistence STAT5 and pro-inflammatory STAT4; IL7R(316-459)-IL2Rbsmall(393-433, 518-551) for pro-persistence; IL7R(316-459)-EGFR(1122-1165) for pro-persistence and anti-exhaustion; IL2Rbsmall(393-433, 518-551)-EGFR(1122-1165) for pro-persistence and anti-exhaustion).

In some embodiments, the constitutively active signaling pathways include activation of a TRAF pathway through activation of TNF receptor associated factors such as TRAF3, TRAF4, TRAF7, and in illustrative embodiments TRAF1, TRAF2, TRAF5, and/or TRAF6. Thus, in certain embodiments, lymphoproliferative elements for use in any of the kits, methods, uses, or compositions herein, are constitutively active and comprise an intracellular signaling domain that activates a Jak/Stat pathway and/or a TRAF pathway. In some embodiments, the constitutively active signaling pathways include activation of PI3K pathways. In some embodiments, the constitutively active signaling pathways include activation of PLC pathways. Thus, in certain embodiments, lymphoproliferative elements for use in any of the kits, methods, uses, or compositions herein, are constitutively active and comprise an intracellular signaling domain that activates a Jak/Stat pathway a TRAF pathway, a PI3K pathway, and/or a PLC pathway. As illustrated therein, where there is a first and a second intracellular signaling domain of a CLE, the first intracellular signaling domain is positioned between the membrane associating motif, for example, a transmembrane domain, and the second intracellular domain.

In some embodiments, the lymphoproliferative elements provided herein include one or more, or all of the binding domains, including those disclosed herein, responsible for signaling found in the corresponding lymphoproliferative element in nature. In some embodiments, the lymphoproliferative elements provided herein include one or more JAK binding domains. In some embodiments, the JAK-binding domain is, or is derived from, EPOR, GP130, PRLR, GHR, GCSFR, or TPOR/MPL. JAK-binding domains from these proteins are known in the art and a skilled artisan will understand how to use them. For example, residues 273-338 of EpoR and residues 478-582 of TpoR are known to be JAK-binding domains. Conserved motifs that are found in intracellular domains of cytokine receptors that are responsible for this signaling are known and are present in certain illustrative lymphoproliferative elements provided herein (see e.g., Morris et al., “The molecular details of cytokine signaling via the JAK/STAT pathway,” Protein Science (2018) 27:1984-2009). The Box1 and Box2 motifs are involved in binding to JAKs and signal transduction, although the Box2 motif presence is not always required for a proliferative signal (Murakami et al. Proc Natl Acad Sci USA. 1991 Dec. 15; 88(24):11349-53; Fukunaga et al. EMBO J. 1991 October; 10(10):2855-65; and O'Neal and Lee. Lymphokine Cytokine Res. 1993 October; 12(5):309-12). Accordingly, in some embodiments a lymphoproliferative element herein is a transgenic Box1-containing cytokine receptor that includes an intracellular domain of a cytokine receptor comprising a Box1 Janus kinase (JAK)-binding motif, optionally a Box2 JAK-binding motif, and a Signal Transducer and Activator of Transcription (STAT) binding motif comprising a tyrosine residue. In some embodiments, a lymphoproliferative element includes two or more JAK-binding motifs, for example three or more or four or more JAK-binding motifs, which in illustrative are the binding motifs found in natural versions of the corresponding lymphoproliferative element.

Intracellular domains from IFNAR1, IFNGR1, IFNLR1, IL2RB, IL4R, IL5RB, IL6R, IL6ST, IL7RA, IL9R, IL10RA, IL21R, IL27R, IL31RA, LIFR, and OSMR are known in the art to activate JAK1 signaling and thus comprise a JAK1 binding motif. Intracellular domains from CRLF2, CSF2RA, CSF2RB, CSF3R, EPOR, GHR, IFNGR2, IL3RA, IL5RA, IL6ST, IL20RA, IL20RB, IL23R, IL27R, LEPR, MPL, and PRLR are known in the art to activate JAK2 and thus comprise a JAK2 binding motif. Intracellular domains from IL2RG are known in the art to activate JAK3 and thus comprise a JAK3 binding motif Intracellular domains from GHR, IFNAR1, IFNAR2, IFNGR1, IFNGR2, IL2RB, IL2RG, IL4R, IL5RA, IL5RB, IL7RA, IL9R, IL21R, IL22RA1, IL31RA, LIFR, MPL, and OSMR are known in the art to activate STAT1. Intracellular domains from IFNAR1 and IFNAR2 are known in the art to activate STAT2. Intracellular domains from GHR, IL2RB, IL2RG, IL6R, IL7RA, IL9R, IL10RA, IL10RB, IL21R, IL22RA1, IL23R, IL27R, IL31RA, LEPR, LIFR, MPL, and OSMR are known in the art to activate STAT3. Intracellular domains from IL12RB1 are known in the art to activate STAT4. Intracellular domains from CSF2RA, CSF2RB, CSF3R, EPOR, GHR, IL2RB, IL2RG, IL3RA, IL4R, IL5RA, IL5RB, IL7RA, IL9R, IL15RA, IL20RA, IL20RB, IL21R, IL22RA1, IL31RA, LIFR, MPL, OSMR, and PRLR are known in the art to activate STAT5. Intracellular domains from IL4R and OSMR are known in the art to activate STAT6. The genes and intracellular domains thereof that are found in a first intracellular domain are the same as the optional second intracellular domain, except that if the first and second intracellular domain are identical, then at least one, and typically both the transmembrane domain and the extracellular domain are not from the same gene.

In some embodiments, a lymphoproliferative element herein can include one or more intracellular signaling domains that include one or more Box1 motifs. In some embodiments, the one or more intracellular signaling domains that include one or more Box1 motifs can be IL7RA (Box1 motif at residues 9-17 of SEQ ID NOs:248 and 249), IL12RB ((Box1 motifs at residues 10-12 of SEQ ID NOs:254 and 255; and residues 107-110 and 139-142 of SEQ ID NO:256), IL31RA (Box1 motifs at residues 12-15 of SEQ ID NOs:275 and 276), CSF2RB (Box1 motif at residues 14-22 of SEQ ID NO:213), IL2RB (Box1 motif at residues 13-21 of SEQ ID NO:240), IL6ST (Box1 motif at residues 10-18 of SEQ ID NO:247), IL2RG (Box1 motif at residues 3-11 of SEQ ID NO:241), IL27RA (Box1 motif at residues 17-25 of SEQ ID NO:273), MPL (Box1 motif at residues 17-20 of SEQ ID NO:283), OSMR (Box1 motif at residues 16-30 of SEQ ID NO:294), IFNAR2 (Box1 motif at residues 23-31 of SEQ ID NO:227), CSF3R, or EPOR (Box1 motif at residues 257-264 of full-length EPOR).

In some embodiments, a lymphoproliferative element herein can include one or more intracellular signaling domains that include one or more Box2 motifs. In some embodiments, the one or more intracellular signaling domains that include one or more Box2 motifs can be MPL (Box2 motif at residues 46-64 in SEQ ID NO:283), IFNAR2 (Box1 motif at residues 37-46 of SEQ ID NO:227), CSF3R, or EPOR (Box2 motif at residues 303-313 of full-length EPOR). EPOR also contains an extended Box2 motif (residues 329-372 of full-length EPOR) important for binding tyrosine kinase receptor KIT, which, in some embodiments, a lymphoproliferative element can include. CSF3R also contains a Box3 motif, which, in some embodiments, a lymphoproliferative element can include.

Some intracellular signaling domains have hydrophobic residues at positions −1, −2, and −6 relative to the Box1 motif, that form a “switch motif,” which is required for cytokine-induced JAK2 activation but not for JAK2 binding (Constantinescu et al. Mol Cell. 2001 February; 7(2):377-85; and Huang et al. Mol Cell. 2001 December; 8(6):1327-38). Accordingly, in certain embodiments, the Box1 motif-containing lymphoproliferative element has a switch motif, which in illustrative embodiments has one or more, and preferably all hydrophobic residues at positions −1, −2, and −6 relative to the Box1 motif. In certain embodiments, the Box1 motif an ICD of a lymphoproliferative element is located proximal to the transmembrane (TM) domain (for example between 5 and 15 or about 10 residues downstream from the TM domain) relative to the Box2 motif, which is located proximal to the transmembrane domain (for example between 10 and 50 residues downstream from the TM domain) relative to the STAT binding motif. The STAT binding motif typically comprising a tyrosine residue, the phosphorylation of which affects binding of a STAT to the STAT binding motif of the lymphoproliferative element. In some embodiments, the ICDs comprising multiple STAT binding motifs where multiple STAT binding motifs are present in a native ICD (e.g., EPO receptor and IL-6 receptor signaling chain (gp130). In some embodiments, the switch motif containing intracellular signaling domain can be MPL (switch motif at residues 11, 15, and 16 of SEQ ID NO:283).

In some embodiments, a lymphoproliferative element herein can include one or more intracellular signaling domains that include one or more phosphorylatable residues, for example, a phosphorylatable serine, threonine, or tyrosine. In some embodiments, the one or more intracellular signaling domains that include one or more phosphorylatable residues can be IL31RA (phosphorylatable tyrosines at residues Y96, Y237, and Y165 of SEQ ID NO:275; not present in SEQ ID NO:276), CD27 (phosphorylatable serine at residue S6 of SEQ ID NO:205), CSF2RB (phosphorylatable tyrosine at residue Y306 of SEQ ID NO:213), IL6ST (phosphorylatable serines at residues S20, S26, S141, S148, S188, and S198 of SEQ ID NO:247), MPL (phosphorylatable tyrosines at residues Y8, Y29, Y78, Y113, and Y118 of SEQ ID NO: 283), CD79B (phosphorylatable tyrosines at residues Y16 and Y27 of SEQ ID NO: 211), OSMR (phosphorylatable serines at residues S65 and S128 of SEQ ID NO:294), or CD3G (phosphorylatable serines at residues S123 and S126 of full-length CD3G). In some embodiments, a lymphoproliferative element that includes a CSF3R intracellular domain can include one, two, three, or all of the tyrosine residues corresponding to Y704, Y729, Y744, and Y764 of full-length CSF3R, various combinations of which have been shown to be important for binding Stat3, SOCS3, Grb2, and p21Ras. In some embodiments, a lymphoproliferative element herein can include one or more intracellular signaling domains that has one or more of its phosphorylatable residues mutated to a phosphomimetic residue, for example, aspartic acid or glutamic acid. In some embodiments, a lymphoproliferative element herein can include one or more intracellular signaling domains that has one or more of its phosphorylatable tyrosines mutated to a non-phosphorylatable residue, for example, alanine, valine, or phenylalanine. In some embodiments, a lymphoproliferative element that includes a CSF3R intracellular domain can include one or more mutations corresponding to T615A and T6181 of full-length CSF3R, which have been shown to increase receptor dimerization and activity.

In some embodiments, a lymphoproliferative element herein can include one or more intracellular signaling domains that include one or more ubiquitination targeting motif residues. In some embodiments, the one or more intracellular signaling domains that include one or more ubiquitination targeting motif residues can be MPL (residues at K40 and K60 of SEQ ID NO:283) or OX40 (residues at K17 and K41 of SEQ ID NO:296). In some embodiments herein, an intracellular domain including ubiquitination targeting motif residues can have one or more of the lysines mutated to arginine or another amino acid.

In some embodiments, a lymphoproliferative element herein can include one or more intracellular signaling domains that include one or more TRAF binding sites. Not to be limited by theory, TRAF1, TRAF2, and TRAF3 binding sites include the amino acid sequence PXQXT (SEQ ID NO:303), where each X can be any amino acid, a distinct TRAF2 binding site includes the consensus sequence SXXE (SEQ ID NO:304) where each X can be any amino acid, and a TRAF6 binding site includes the consensus sequence QXPXEX (SEQ ID NO:305). In some embodiments, the one or more intracellular signaling domains that include one or more TRAF binding sites can be CD40 (binding sites for TRAF1, TRAF2, and TRAF3 at residues 35-39 of SEQ ID NO:208; TRAF2 binding site at residues 57-60 of SEQ ID NO:208; TRAF6 binding site at residues 16-21 of SEQ ID NO:208), or OX40 (TRAF1, TRAF2, TRAF3, and TRAF5 binding motif at residues 20-27 of SEQ ID NO:296).

In some embodiments, a lymphoproliferative element herein can include one or more intracellular signaling domains that include a TIR domain. In some embodiments, the one or more intracellular signaling domains that include a TIR domains can be IL17RE (TIR domain at residues 13-136 of SEQ ID NO:265), IL18R1 (TIR domain at residues 28-170 of SEQ ID NO:266), or MyD88 (TIR domain at residues 160-304 of SEQ ID NO:284).

In some embodiments, a lymphoproliferative element herein can include one or more intracellular signaling domains that include a PI3K binding motif domain. In some embodiments, the one or more intracellular signaling domains that include a PI3K binding motif can be CD28 (PI3K binding motifs at residues 12-15 of SEQ ID NOs:206 and 207, which also binds Grb2), ICOS (PI3K binding motif at residues 19-22 of SEQ ID NO:225, which can be mutated F21Q to increase IL-2 production and/or to bind Grb2), OX40 (p85 PI3K binding motif at residues 34-57 of full-length OX40)

In some embodiments, a lymphoproliferative element herein can include one or more intracellular signaling domains that include a dileucine motif. In some embodiments, the one or more intracellular signaling domains that include a dileucine motif can be IFNGR2 (dileucine motif at residues 8-9 of SEQ ID NO:230) or CD3G (dileucine motif at residues 131-132 of full-length CD3G). In some embodiments, one or both of the residues in the dileucine motif can be mutated.

In some embodiments, a lymphoproliferative element herein can include one or more intracellular signaling domains that include one or more N-terminal death domains. In some embodiments, the one or more intracellular signaling domains that include one or more N-terminal death domains can be MyD88 (N-terminal death domain at residues 29-106 of SEQ ID NO:284) or a TNFR. The cytoplasmic domains of TNF receptors (TNFRs), which in illustrative embodiments can be TNFRSF4, TNFRSF8, TNFRSF9, TNFRSF14, or TNFRSF18, can recruit signaling molecules, including TRAFs (TNF receptor-associated factors) and/or “death domain” (DD) molecules. The domains, motifs, and point mutations of TNFRs that induce proliferation and/or survival of T cells and/or NK cells are known in the art and a skilled artisan can identify corresponding domains, motifs, and point mutations in TNFR polypeptides. A skilled artisan will be able to identify the TRAF- and/or DD-binding motif in the different TNFR families using, for example, sequence alignments to known binding motifs. In some embodiments, a lymphoproliferative element that includes a TNFR intracellular domain can include one or more TRAF-binding motifs. In some embodiments, a lymphoproliferative element that includes a TNFR intracellular domain does not include a DD-binding motif, or has one or more DD-binding motifs deleted or mutated within the intracellular domain. In some embodiments, a lymphoproliferative element that includes a TNFR intracellular domain can recruit TRADD and/or TRAF2. TNFRs also include cysteine-rich domains (CRDs) that are important for ligand binding (Locksley R M et al. Cell. 2001 Feb. 23; 104(4):487-501). In some embodiments, a lymphoproliferative element that includes a TNFR intracellular domain does not include a TNFR CRD.

In some embodiments, a lymphoproliferative element herein can include one or more intracellular signaling domains that include one or more intermediate domains that interact with IL-1R associated kinase. In some embodiments, the one or more intracellular signaling domains that include one or more intermediate domains can be MyD88 (intermediate domain at residues 107-156 of SEQ ID NO:284).

In some embodiments, a lymphoproliferative element that includes an intracellular domain from IL7RA can include one or more of the S region or T region (S region at residues 359-394 and T region at residues Y401, Y449, and Y456 of full-length IL7RA). In some embodiments of lymphoproliferative elements that comprise an intracellular domain from IL7RA, the lymphoproliferative element comprises a second intracellular domain from a receptor other than IL7RA. In some embodiments, the second intracellular domain is derived from TNFRSF8. In some embodiments, the second intracellular domain is derived from IL2Rβ. In some embodiments, the second intracellular domain is derived from IL12Rβ2. In some embodiments of lymphoproliferative elements that comprise an intracellular domain from IL7RA, the transmembrane domain is derived from EpoR, GP130, PrlR, GHR, GCSFR, or TPOR/MPL. In some embodiments of lymphoproliferative elements that comprise an intracellular domain from IL2Rβ, the transmembrane domain is derived from EpoR, GP130, PrlR, GHR, GCSFR, or TPOR/MPL. In some embodiments of lymphoproliferative elements that comprise an intracellular domain from IL12Rβ2, the transmembrane domain is derived from EpoR, GP130, PrlR, GHR, GCSFR, or TPOR/MPL. In some embodiments of lymphoproliferative elements that comprise an intracellular domain from IL2Rβ, IL7RA, or IL2Rβ, the transmembrane domain comprises amino acids 478-582 of the naturally occurring TPOR/MPL. In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD11c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIG1.

In some embodiments, a lymphoproliferative element that includes an intracellular domain from MyD88 can include one or more of the mutations L93P, R193C, and L265P in full-length MyD88 (mutations L93P, R196C, and L260P of SEQ ID NO:284). In illustrative embodiments of lymphoproliferative elements that include a first intracellular domain derived from MyD88, the second intracellular domain can be derived from TNFRSF4 or TNFRSF8. In other illustrative embodiments of lymphoproliferative elements that include a first intracellular domain derived from MyD88, the second intracellular domain can be other than an intracellular domain derived from a CD28 family member (e.g., CD28, ICOS), Pattern Recognition Receptor, a C-reactive protein receptor, a TNF receptor, or an HSP receptor.

In some embodiments, a cell expressing the lymphoproliferative element comprising an intracellular and transmembrane domain of MPL can be contacted with or exposed to eltrombopag, or a patient or subject to which such a cell has been infused can be treated with eltrombopag. Not to be limited by theory, eltrombopag binds to the transmembrane domain of MPL and induces the activation of the intracellular domain of MPL.

The domains, motifs, and point mutations of MPL that induce proliferation and/or survival of T cells and/or NK cells are known in the art and a skilled artisan can identify corresponding domains, motifs, and point mutations in MPL polypeptides, some of which are discussed in this paragraph. Deletion of the region encompassing amino acids 70-95 in SEQ ID NO:283 was shown to support viral transformation in the context of v-mpl (Benit et al. J Virol. 1994 August; 68(8):5270-4), thus indicating that this region is not necessary for the function of mpl in this context. Morello et al. Blood 1995 July; 86(8):557-71 used the same deletion to show that this region was not required for stimulating transcription for a hematopoietin receptor-responsive CAT reporter gene construct and furthermore saw that this deletion resulted in slightly enhanced transcription expected for removal of a nonessential and negative element in this region as suggested by Drachman and Kaushansky. Thus, in some embodiments, a MPL intracellular signaling domain does not comprise the region comprising amino acids 70-95 in SEQ ID NO:283. Using computer simulations, Lee et al. found clinically relevant mutations in the transmembrane domain of MPL should activate MPL with the following order of activating effects: W515K (corresponding to the amino acid substitution W2K of SEQ ID NO: 283) >S505A (corresponding to the amino acid substitution S14A of SEQ ID NO: 187)>W5151 (corresponding to the amino acid substitution W2I of SEQ ID NO: 283)>S505N (corresponding to the amino acid substitution S14N of SEQ ID NO: 187, which was tested as part T075 (SEQ ID NO: 188)) (Lee et. al. PLoS One. 2011; 6(8): e23396). The simulations predicted these mutations could cause constitutive activation of JAK2, the kinase partner of MPL. In some embodiments, the intracellular portion of MPL can include one or more, or all the domains and motifs described herein that are present in SEQ ID NO: 283. In some embodiments, a transmembrane portion of MPL can include one or more, or all the domains and motifs described herein that are present in SEQ ID NO: 187. In illustrative embodiments of lymphoproliferative elements that include a first intracellular domain derived from MPL, the second intracellular domain can be derived from CD79B.

In some embodiments, a lymphoproliferative element that includes an PRLR intracellular domain can include the growth hormone receptor binding domain of PRLR and any known mutations (growth hormone receptor binding domain at residues 28-104 of SEQ ID NO:295).

In some embodiments, a lymphoproliferative element that includes an ICOS intracellular domain can include a calcium-signaling motif (calcium-signaling motif at residues 5-8 of SEQ ID NO:225). In some embodiments, a lymphoproliferative element that includes an ICOS intracellular domain can include at least one of a first and second conserved motif (first and second conserved motifs at residues 9-18 and 24-30, respectively, of SEQ ID NO:225). In some embodiments, a lymphoproliferative element that includes an ICOS intracellular domain does not include at least one of the first and second conserved motif.

EPOR also contains a short segment important for EPOR internalization (residues 267-276 of full-length EPOR). In some embodiments, a lymphoproliferative element that includes an EPOR intracellular domain does not include the internalization segment.

The domains, motifs, and point mutations of intracellular signaling domains that induce proliferation and/or survival of T cells and/or NK cells are known in the art and a skilled artisan can identify corresponding domains, motifs, and point mutations in polypeptides, some of which are above, and a skilled artisan can identify corresponding domains, motifs, and point mutations in other polypeptides. A skilled artisan will be able to identify these domains, motifs, and point mutations in similar polypeptides using, for example, sequence alignments to known binding motifs. In some embodiments, a lymphoproliferative element herein can include any, for example, one or more up to all of the domains, motifs, and mutations of an intracellular signaling domain disclosed herein or otherwise known to induce proliferation and/or survival of T cells and/or NK cells.

In another embodiment, the LE provides, is capable of providing and/or possesses the property of (or a cell modified, genetically modified, and/or transduced with the LE is capable of providing, is adapted for, possesses the property of, and/or is modified for) driving T cell expansion in vivo. In some embodiments, the lymphoproliferative element can include any of the sequences listed in Table 1 (SEQ ID NOs:84-302). In some embodiments, the lymphoproliferative element can a sequence with at least 90% sequence identity to any of the sequences listed in Table 1 (SEQ ID NOs:84-302). In some embodiments, the lymphoproliferative element can a sequence with at least 95% sequence identity to any of the sequences listed in Table 1 (SEQ ID NOs:84-302). In some embodiments, the lymphoproliferative element can a sequence with at least 96% sequence identity to any of the sequences listed in Table 1 (SEQ ID NOs:84-302). In some embodiments, the lymphoproliferative element can a sequence with at least 97% sequence identity to any of the sequences listed in Table 1 (SEQ ID NOs:84-302). In some embodiments, the lymphoproliferative element can a sequence with at least 98% sequence identity to any of the sequences listed in Table 1 (SEQ ID NOs:84-302). In some embodiments, the lymphoproliferative element can a sequence with at least 99% sequence identity to any of the sequences listed in Table 1 (SEQ ID NOs:84-302). Table 1 shows the parts, names (including gene names), and amino acid sequences for domains in CLEs. CLEs can include in certain illustrative embodiments, an extracellular domain (ECD) (denoted P1), a transmembrane (TM) domain (denoted P2), a first intracellular domain (ICD) (denoted P3), and a second ICD (denoted P4). In some embodiment, CLEs provided herein can be heterodimeric CLEs comprised of two different LE or CLE polypeptides that each comprise a TM domain and an ICD and, in illustrative embodiments, an ECD, wherein the TM or ECD of each LE polypeptide of the heterodimer comprises a dimerizing motif that can bind to the other (i.e., complementary dimerizing motifs). In some embodiments of these heterodimeric CLEs, the ICD of one polypeptide is any of the first ICDs called out herein and the ICD of the other polypeptide of the homodimer is any of the second ICDs called out herein. In some embodiments of these heterodimeric CLEs, the ICD of one polypeptide is one of the P3 ICDs in Table 1, and the ICD of the other polypeptide of the heterodimer comprises a corresponding P4 ICD of Table 1. In certain illustrative embodiments, retroviruses encoding such heterodimeric CLEs can be directly administered to a subject. In certain illustrative embodiments, retroviruses encoding such heterodimeric CLEs can comprise membrane-bound cytokines.

Typically, the lymphoproliferative element includes a first intracellular domain. In illustrative embodiments, the first intracellular domain can include any of the parts listed as S036 to S0216 or in Table 1, or functional mutants and/or fragments thereof. In some embodiments, the lymphoproliferative element can include a second intracellular domain. In illustrative embodiments, the second intracellular domain can include any of the parts listed as S036 to S0216 or in Table 1, or functional mutants and/or fragments thereof. In some embodiments, the lymphoproliferative element can include an extracellular domain. In illustrative embodiments, the extracellular domain can include any of the sequences of parts listed as M001 to M049 or E006 to E015 in Table 1, or functional mutants and/or fragments thereof. In some embodiments, the lymphoproliferative element can include a transmembrane domain. In illustrative embodiments, the transmembrane domain can include any of the parts listed as M001 to M049 or T001 to T082 in Table 1, or functional mutants and/or fragments thereof. In some embodiments, the lymphoproliferative element can be a fusion of an extracellular/transmembrane domain (M001 to M049 in Table 1), a first intracellular domain (S036 to S0216 in Table 1), and a second intracellular domain (S036 to S216 in Table 1). In some embodiments, the lymphoproliferative element can be a fusion of an extracellular domain (E006 to E016 in Table 1), a transmembrane domain (T001 to T082 in Table 1), a first intracellular domain (S036 to S0216 in Table 1), and a second intracellular domain (S036 to S0216 in Table 1). For example, the lymphoproliferative element can be a fusion of E006, T001, S036, and S216, also written as E006-T001-S036-S216). In illustrative embodiments, the lymphoproliferative element can be the fusion E010-T072-S192-S212, E007-T054-S197-S212, E006-T006-S194-S211, E009-T073-S062-S053, E008-T001-S121-S212, E006-T044-S186-S053, or E006-T016-S186-S050.

In illustrative embodiments, the intracellular domain of an LE, or the first intracellular domain in an LE that has two or more intracellular domains, is other than a functional intracellular activating domain from an ITAM-containing intracellular domain, for example, an intracellular domain from CD3Z, CD3D, CD3E, CD3G, CD79A, CD79B, DAP12, FCER1G, FCGR2A, FCGR2C, DAP10/CD28, or ZAP70, and in a further illustrative subembodiment, CD3z. In illustrative embodiments, the extracellular domain of an LE does not comprise a single-chain variable fragment (scFv). In further illustrative embodiments, the extracellular domain of an LE that upon binding to a binding partner activates an LE, does not comprise a single-chain variable fragment (scFv). A CLE does not comprise both an ASTR and an activation domain from CD3Z, CD3D, CD3E, CD3G, CD79A, CD79B, DAP12, FCER1G, FCGR2A, FCGR2C, DAP10/CD28, or ZAP70. If an LE does include an ASTR (and not an activation domain in the previous list), the ASTR of an LE in illustrative embodiments does not include an scFv. In some embodiments, a lymphoproliferative element does not include an extracellular domain.

In some embodiments, the lymphoproliferative element, and in illustrative embodiments CLE, is not covalently attached to a cytokine. In some aspects, a lymphoproliferative element, and in illustrative embodiments CLE, comprises a cytokine polypeptide covalently linked to its cognate receptor. In either of these embodiments, the CLE can be constitutively active and typically constitutively activates the same Jak/STAT and/or TRAF pathways as the corresponding activated wild-type cytokine receptor. In some embodiments, the chimeric cytokine receptor is an interleukin. In some embodiments, the CLE is IL-7 covalently linked to IL7RA or IL-15 covalently linked to IL15RA. In other embodiments, the CLE is other than IL-15 covalently linked to IL15RA. In other aspects, the CLE comprises a cytokine polypeptide covalently linked to only a portion of its cognate receptor that includes a functional portion of the extracellular domain capable of binding the cytokine polypeptide, the transmembrane domain and/or intracellular domain are from heterologous polypeptides, and the CLE is constitutively active. In one embodiment, the CLE is IL-7 covalently linked to the extracellular and transmembrane domains of IL7RA, and the intracellular domain from IL2RB. In another embodiment, the CLE is a cytokine polypeptide covalently linked to a portion of its cognate receptor that includes a functional portion of the extracellular domain capable of binding the cytokine polypeptide, a heterologous transmembrane domain, and lymphoproliferative element intracellular domain provided herein. In some embodiments, the lymphoproliferative element is a cytokine receptor that is not tethered to a cytokine.

In some aspects, the lymphoproliferative element is capable of binding to soluble cytokines or growth factors and such binding is required for activity. In certain illustrative embodiments, the lymphoproliferative element is constitutively active, and thus does not require binding to a soluble growth factor or cytokine for activity. Typically, constitutively active lymphoproliferative elements do not bind soluble cytokines or growth factors. In some embodiments, the lymphoproliferative element is a chimera comprising an extracellular binding domain from one receptor and the intracellular signaling domain from a different receptor. In some embodiments the CLE is an inverted receptor that is activated upon binding of a ligand that would inhibit proliferation and/or survival when bound to its natural receptor, but instead leads to proliferation and/or survival upon activating the CLE. In some embodiments, inverted receptors include chimeras that comprise an extracellular ligand binding domain from IL4Ra and an intracellular domain from IL7Ra or IL21. Other embodiments of inverted cytokine receptors include chimeras that comprise an extracellular ligand binding domain from a receptor that would inhibit proliferation and/or survival when bound to its natural ligand, such as receptors for IL-4, IL-10, IL-13, or TGFb, and any lymphoproliferative element intracellular domain disclosed herein. In illustrative aspects, the lymphoproliferative element does not bind a cytokine. In further illustrative aspects, the lymphoproliferative element does not bind any ligand. In illustrative embodiments, the lymphoproliferative elements that do not bind any ligand are constitutively dimerized or otherwise multimerized and are constitutively active. In illustrative embodiments of any of the methods and compositions provided herein that include a lymphoproliferative element, the intracellular domain can be derived from an intracellular portion of the transmembrane protein of the TNF receptor family, CD40. The domains, motifs, and point mutations of CD40 that induce proliferation and/or survival of T cells and/or NK cells are known in the art and a skilled artisan can identify corresponding domains, motifs, and point mutations in CD40 polypeptides, some of which are discussed in this paragraph. The CD40 protein contains several binding sites for TRAF proteins. Not to be limited by theory, binding sites for TRAF1, TRAF2, and TRAF3 are located at the membrane distal domain of the intracellular portion of CD40 and include the amino acid sequence PXQXT (SEQ ID NO:303) where each X can be any amino acid, (corresponding to amino acids 35-39 of SEQ ID NO:208) (Elgueta et al. Immunol Rev. 2009 May; 229(1):152-72). TRAF2 has also been shown to bind to the consensus sequence SXXE (SEQ ID NO:304) where each X can be any amino acid, (corresponding to amino acids 57-60 of SEQ ID NO:208) (Elgueta et al. Immunol Rev. 2009 May; 229(1):152-72). A distinct binding site for TRAF6 is situated at the membrane proximal domain of intracellular portion of CD40 and includes the consensus sequence QXPXEX (SEQ ID NO:305) where each X can be any amino acid (corresponding to amino acids 16-21 of SEQ ID NO:208) (Lu et al. J Biol Chem. 2003 Nov. 14; 278(46):45414-8). In illustrative embodiments, the intracellular portion of the transmembrane protein CD40 can include all the binding sites for the TRAF proteins. The TRAF binding sites are known in the art and a skilled artisan will be able to identify corresponding TRAF binding sites in similar CD40 polypeptides. In some embodiments, a suitable intracellular domain can include a domain with at least 50%, 60%, 70%, 75%, 80%, 85%, 90%, 95%, 96%, 97%, 98%, 99% or 100% sequence identity to a stretch of at least 10, 15, 20, or all of the amino acids in SEQ ID NO:208 or SEQ ID NO:209. In some embodiments, the intracellular domain derived from CD40 has a length of from about 30 amino acids (aa) to about 35 aa, from about 35 aa to about 40 aa, from about 40 aa to about 45 aa, from about 45 aa to about 50 aa, from about 50 aa to about 55 aa, from about 55 aa to about 60 aa, or from about 60 aa to about 65 aa. In illustrative embodiments, the intracellular domain derived from CD40 has a length of from about 30 aa to about 66 aa, for example, 30 aa to 65 aa, or 50 aa to 66 aa. In illustrative embodiments of lymphoproliferative elements that include a first intracellular domain derived from CD40, the second intracellular domain can be other than an intracellular domain derived from MyD88, a CD28 family member (e.g., CD28, ICOS), Pattern Recognition Receptor, a C-reactive protein receptor (i.e., Nodi, Nod2, PtX3-R), a TNF receptor, CD40, RANK/TRANCE-R, OX40, 4-1BB), an HSP receptor (Lox-1 and CD91), or CD28. In certain embodiments one intracellular domain of a CLE herein is the intracellular domain from CD40, or a functional fragment thereof, and another intracellular domain of the CLE is the intracellular domain of MPL, or a functional fragment thereof. Pattern Recognition Receptors include, but are not limited to endocytic pattern-recognition receptors (i.e., mannose receptors, scavenger receptors (i.e., Mac-1, LRP, peptidoglycan, teichoic acids, toxins, CD11c/CR4)); external signal pattern-recognition receptors (Toll-like receptors (TLR1, TLR2, TLR3, TLR4, TLR5, TLR6, TLR7, TLR8, TLR9, TLR10), peptidoglycan recognition protein, (PGRPs bind bacterial peptidoglycan, and CD14); internal signal pattern-recognition receptors (i.e., NOD-receptors 1 & 2), and RIGi.

In another embodiment, the LE provides, is capable of providing and/or possesses the property of (or a cell genetically modified and/or transduced with the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold expansion, or between 1.5 fold and 25-fold expansion, or between 2-fold and 20-fold expansion, or between 2-fold and 15-fold expansion, or between 5-fold and 25-fold expansion, or between 5-fold and 20-fold expansion, or between 5-fold and 15-fold expansion, of pre-activated PBMCs transduced with a nucleic acid encoding the LE when transduced along with an anti-CD19 CAR comprising a CD3 zeta intracellular activating domain but no co-stimulatory domain, between day 7 and day 21, 28, 35, and/or 42 of in vitro culturing in the absence of exogenously added cytokines. In some embodiments, the test is performed in the presence of PBMCs, for example at a 1:1 ratio of transduced cells to PBMCs, which can be for example, from a matched donor, and in some embodiments, the test is performed in the absence of PBMCs. In some embodiments, the analysis of expansion for any of these tests is performed as illustrated in WO2019/055946. In some embodiments, the test can include a further statistical test and a cut-off such as a P value below 0.1, 0.05, or 0.01, wherein a test polypeptide or nucleic acid encoding the same, needs to meet one or both thresholds (i.e., fold expansion and statistical cutoff).

In another embodiment, the LE provides, is capable of providing and/or possesses the property of (or a cell genetically modified and/or transduced with the LE is capable of providing, is adapted for, possesses the property of, and/or is modified for) driving T cell expansion in vivo. For example, the in vivo test can utilize a mouse model and measure T cell expansion at 15 to 25 days in vivo, or at 19 to 21 days in vivo, or at approximately 21 days in vivo, after T cells are contacted with lentiviral vectors encoding the LEs, are introduced into the mice, as disclosed in WO2019/055946.

In some embodiments, improved or enhanced survival, expansion, and/or proliferation can be shown as an increase in the number of cells determined by sequencing DNA from cells transduced with retroviral particle (e.g., lentiviral particle) having a genome encoding CLEs and counting the occurrences of sequences present in unique identifiers from each CLE. In some embodiments, improved survival and/or improved expansion can be determined by counting the cells directly, for example with a hemocytometer or a cell counter, at each time point. In some embodiments, improved survival and/or improved expansion and/or enrichment can be calculated by dividing the number of cells at the later time point (day 21, 28, 35, and/or day 45) by the number of cells at day 7 for each construct. In some embodiments, the cells can be counted by hemocytometer or cell counters. In some embodiments, the enrichment level determined using the nucleic acid counts or the cell counts of each specific test construct can be normalized to the enrichment level of the respective control construct, i.e., the construct with the same extracellular domain and transmembrane domain but lacking the intracellular domains present in the test construct. In these embodiments, the LE encoded in the construct provides (or a cell genetically modified and/or transduced with a retroviral particle (e.g., lentiviral particle) having a genome encoding the LE is capable of providing, is adapted for, possesses the property of, and/or is modified for) at least a 1.5-fold, 2-fold, 3-fold, 4-fold, 5-fold, 6-fold, 7-fold, 8-fold, 9-fold, or 10-fold normalized enrichment level, or between 1.5 fold and 25-fold normalized enrichment level, or between 3-fold and 20-fold normalized enrichment level, or between 5-fold and 25-fold normalized enrichment level, or between 5-fold and 20-fold normalized enrichment level, or between 5-fold and 15-fold normalized enrichment level.

In some embodiments, a lymphoproliferative element, including a CLE, comprises an intracellular activating domain as disclosed hereinabove. In some illustrative embodiments a lymphoproliferative element is a CLE comprising an intracellular activating domain comprising an ITAM-containing domain, as such, the CLE can comprise an intracellular activating domain having at least 80%, 90%, 95%, 98%, or 100% sequence identity to the CD3Z, CD3D, CD3E, CD3G, CD79A, CD79B, DAP12, FCER1G, FCGR2A, FCGR2C, DAP10/CD28, or ZAP70 domains provided herein wherein the CLE does not comprise an ASTR.

In some embodiments, one or more domains of a lymphoproliferative element is fused to a modulatory domain, such as a co-stimulatory domain, and/or an intracellular activating domain of a CAR. In some embodiments of the composition and method aspects for transducing lymphocytes in whole blood, one or more intracellular domains of a lymphoproliferative element can be part of the same polypeptide as a CAR or can be fused and optionally functionally connected to some components of CARs. In still other embodiments, an engineered signaling polypeptide can include an ASTR, an intracellular activation domain (such as a CD3 zeta signaling domain), a co-stimulatory domain, and a lymphoproliferative domain. Further details regarding co-stimulatory domains, intracellular activating domains, ASTRs and other CAR domains, are disclosed elsewhere herein.

In some embodiments, CLEs include both an extracellular portion and a transmembrane portion that is from the same protein, in illustrative embodiments the same receptor, either of which in illustrative embodiments is a mutant, thus forming an extracellular and transmembrane domain. These domains can be from a cytokine receptor, or a mutant thereof, or a hormone receptor, or a mutant thereof in some embodiments that have been reported to be constitutively active when expressed at least in some cell types. In illustrative embodiments, such extracellular and transmembrane domains do not include a ligand binding region. It is believed that such domains do not bind a ligand when present in CLEs and expressed in B cells, T cells, and/or NK cells. Mutations in such receptor mutants can occur in the transmembrane region or in the extracellular juxtamembrane region. Not to be limited by theory, a mutation in at least some extracellular—transmembrane domains of CLEs provided herein, are responsible for signaling of the CLE in the absence of ligand, by bringing activating chains together that are not normally together, or by changing the confirmation of a linked transmembrane and/or intracellular domain.

In some embodiments, the dimerizing moiety of a CLE can comprise an epitope recognized by an antibody, such as, for example, a clinical antibody, which in illustrative embodiments can be an approved biologic. As a non-limiting example, an ECD of an LE herein can include an ECD that includes one or multiple tandem copies of a PD-1 epitope and/or a CTLA-4 epitope. As such, an anti-PD-1 antibody and/or an anti-CTLA-4 antibody respectively, for example a clinical anti-PD-1 antibody or a clinical anti-CTLA-4 antibody, can be used to dimerize and activate such an LE. Not to be limited by theory, such embodiments might provide an advantage for anti-PD-1 administration in both blocking interactions of PD-1 for example expressed on CAR-T cells, with PDL-1 expressed on tumor cells, and driving proliferation and/or survival signal in the CAR-T cells through dimerization of a homodimeric or a heterodimeric LE. In some embodiments, where LEs comprising a PD-1 epitope-containing ECD is used, an anti-PD1 antibody, for example, nivolumab or pembrolizumab, can be provided and/or administered to a subject as part of a combination therapy. The ECD can be referred to herein as the ectodomain. In some embodiments, the ectodomain can comprise a PD-1 polypeptide, such as any of the polypeptides of Table 2 below −1 or Table 1 of WO2020180664A1 (incorporated by reference herein in its entirety) whose ectoderm include “PD1” in its name.

TABLE 2
PD-1 sequences

| Ectodomain | Amino acid sequence | SEQ ID NO: |
|---|---|---|
| High-affinity (HA) PD-1 | PGWFLDSPDRPWNPPTFSPA | 1034 |
| ectodomain | LLVVTEGDNATFTCSFSNTS | |
| | ESFHVIWHRESPSGQTDTLA | |
| | AFPEDRSQPGQDCRFRVTQL | |
| | PNGRDFHMSVVRARRNDSG | |
| | TYVCGVISLAPKIQIKESLRA | |
| | ELRVTERRAEVPTAHPSPSP | |
| | RPAGQFQTLVVGVVGGLLG | |
| | SLVLLVWVLAVICSRAARG | |
| | TIGARRTGQ | |
| FKBP (F36V) | MGVQVETISPGDGRTFPKRG | 1035 |
| | QTCVVHYTGMLEDGKKVD | |
| | SSRDRNKPFKFMLGKQEVIR | |
| | GWEEGVAQMSVGQRAKLTI | |
| | SPDYAYGATGHPGIIPPHAT | |
| | LVFDVELLKLE | |
| Wildtype (WT) PD-1 | PGWFLDSPDRPWNPPTFSPA | 1036 |
| ectodomain (PD-1 (21-170)) | LLVVTEGDNATFTCSFSNTS | |
| | ESFVLNWYRMSPSNQTDKL | |
| | AAFPEDRSQPGQDCRFRVTQ | |
| | LPNGRDFHMSVVRARRNDS | |
| | GTYLCGAISLAPKAQIKESL | |
| | RAELRVTERRAEVPTAHPSP | |
| | SPRPAGQFQTLV | |
| High-affinity (HA) PD-1 | PGWFLDSPDRPWNPPTFSPA | 1037 |
| ectodomain (21-170) | LLVVTEGDNATFTCSFSNTS | |
| | ESFHVIWHRESPSGQTDTLA | |
| | AFPEDRSQPGQDCRFRVTQL | |
| | PNGRDFHMSVVRARRNDSG | |
| | TYVCGVISLAPKIQIKESLRA | |
| | ELRVTERRAEVPTAHPSPSP | |
| | RPAGQFQTLV | |
| 2x WTPD-1 ectodomain (PD- | PGWFLDSPDRPWNPPTFSPA | 1038 |
| 1(21-170)-G-PD-1(21-170)-G- | LLVVTEGDNATFTCSFSNTS | |
| PD-1(21-170)) | ESFVLNWYRMSPSNQTDKL | |
| | AAFPEDRSQPGQDCRFRVTQ | |
| | LPNGRDFHMSVVRARRNDS | |
| | GTYLCGAISLAPKAQIKESL | |
| | RAELRVTERRAEVPTAHPSP | |
| | SPRPAGQFQTLVGPGWFLDS | |
| | PDRPWNPPTFSPALLVVTEG | |
| | DNATFTCSFSNTSESFVLNW | |
| | YRMSPSNQTDKLAAFPEDRS | |
| | QPGQDCRFRVTQLPNGRDF | |
| | HMSVVRARRNDSGTYLCGA | |
| | ISLAPKAQIKESLRAELRVTE | |
| | RRAEVPTAHPSPSPRPAGQF | |
| | QTLV | |
| 3x WTPD-1 ectodomain (PD- | PGWFLDSPDRPWNPPTFSPA | 1039 |
| 1(21-170)-G-PD-1(21-170)-G- | LLVVTEGDNATFTCSFSNTS | |
| PD-1(21-170)) | ESFVLNWYRMSPSNQTDKL | |
| | AAFPEDRSQPGQDCRFRVTQ | |
| | LPNGRDFHMSVVRARRNDS | |
| | GTYLCGAISLAPKAQIKESL | |
| | RAELRVTERRAEVPTAHPSP | |
| | SPRPAGQFQTLVGPGWFLDS | |
| | PDRPWNPPTFSPALLVVTEG | |
| | DNATFTCSFSNTSESFVLNW | |
| | YRMSPSNQTDKLAAFPEDRS | |
| | QPGQDCRFRVTQLPNGRDF | |
| | HMSVVRARRNDSGTYLCGA | |
| | ISLAPKAQIKESLRAELRVTE | |
| | RRAEVPTAHPSPSPRPAGQF | |
| | QTLVGPGWFLDSPDRPWNP | |
| | PTFSPALLVVTEGDNATFTC | |
| | SFSNTSESFVLNWYRMSPSN | |
| | QTDKLAAFPEDRSQPGQDC | |
| | RFRVTQLPNGRDFHMSVVR | |
| | ARRNDSGTYLCGAISLAPKA | |
| | QIKESLRAELRVTERRAEVP | |
| | TAHPSP SPRPAGQFQTLV | |

In some embodiments, an ECD of an LE herein can include an ECD that includes one or multiple tandem copies of a TIGIT epitope. As such, an anti-TIGIT antibody, for example, a clinical anti-TIGIT antibody, can be used to dimerize and activate such an LE. Not to be limited by theory, such embodiments might provide an advantage for anti-TIGIT administration in blocking interactions of TIGIT, and driving proliferation and/or survival signal in the CAR-T cells through dimerization of a homodimeric or a heterodimeric LE. In some embodiments, where LEs comprising a TIGIT epitope-containing ECD is used, an anti-TIGIT antibody can be provided and/or administered to a subject as part of a combination therapy.

In some embodiments, the dimerizing moiety of a CLE can comprise an anti-idiotype extracellular recognition domain of any of the anti-idiotype polypeptides herein. As such, anti-idiotype polypeptides containing an anti-idiotype extracellular domain can be CLEs. For example, the extracellular recognition domain attached to such a CLE can dimerize upon binding of the target antibody or antibody mimetic, as disclosed elsewhere herein. In other words, in some embodiments, the CLE is part of a fusion polypeptide including an anti-idiotype polypeptide, and the fusion polypeptide is dimerized through binding of the target antibody or antibody mimetic to the anti-idiotype extracellular recognition domain. In illustrative embodiments, the CLE is not constitutively active, but rather is activated upon dimerization induced by binding of a target antibody to 2 anti-idiotype polypeptides that bind the idiotype of the target antibody. In these embodiments, the target antibody typically does not induce cytotoxicity.

In some embodiments, a lymphoproliferative element provided herein comprises an extracellular domain, and in illustrative embodiments, the extracellular domain comprises a dimerizing motif. In illustrative embodiments of this aspect, the extracellular domain comprises a leucine zipper. In some embodiments, the leucine zipper is from a jun polypeptide, for example c-jun. In certain embodiments the c-jun polypeptide is the c-jun polypeptide region of ECD-11.

An extracellular domain with a dimerizing moiety can also serve a function of connecting a cell tag polypeptide, such as an anti-idiotype extracellular recognition domain of an anti-idiotype polypeptide to a cell expressing a CLE. Accordingly, in such embodiments, the dimerizing motif can serve the function of a stalk connecting an anti-idiotype extracellular recognition domain to a membrane association domain, which in LEs and CLEs is typically a transmembrane domain. Such embodiments provide an advantage of having a transmembrane domain and dimerization motif that anchor an anti-idiotype domain to a cell, while retaining their function in an LE. In some embodiments, such embodiments provide the advantage of providing for tetramerization of an intracellular domain by constitutive dimerization through an LE dimerization motif and inducible dimerization, which would form tetramers, upon binding of a target antibody that has the idiotype recognized by the anti-idiotype extracellular recognition domain. In these embodiments, the target antibody typically does not induce cytotoxicity but rather serves to tetramerize dimerized ICDs. These are useful in some apoptosis-inducing embodiments as described in other sections herein.

In some embodiments, the dimerizing agent can be located intracellularly rather than extracellularly. In some embodiments, more than one or multiples of dimerizing domains can be used. In any aspects or embodiments wherein the extracellular domain of a CLE comprises a dimerizing motif, the dimerizing motif can be selected from the group consisting of: a leucine zipper motif-containing polypeptide, CD69, CD71, CD72, CD96, Cd105, Cd161, Cd162, Cd249, CD271, and Cd324, as well as mutants and/or active fragments thereof that retain the ability to dimerize. In any of the aspects and embodiments herein wherein the extracellular domain of a CLE comprises a dimerizing motif, the dimerizing motif can require a dimerizing agent, and the dimerizing motif and associated dimerizing agent can be selected from the group consisting of FKBP and rapamycin or analogs thereof (e.g., AP1903), GyrB and coumermycin or analogs thereof, DHFR and methotrexate or analogs thereof, or DmrB and AP20187 or analogs thereof, as well as mutants and/or active fragments of the recited dimerizing proteins that retain the ability to dimerize. In some aspects and illustrative embodiments, a lymphoproliferative element is constitutively active, and is other than a lymphoproliferative element that requires a dimerizing agent for activation.

Extracellular domains for embodiments where extracellular domains have a dimerizing motif, are long enough to form dimers, such as leucine zipper dimers. As such, extracellular domains that include a dimerizing moiety can be from 15 to 100, 20 to 50, 30 to 45, or 35 to 40 amino acids, of in illustrative embodiments is a c-Jun portion of a c-Jun extracellular domain. Extracellular domains of polypeptides that include a dimerizing moiety, may not retain other functionalities. For example, for leucine zippers embodiments, such leucine zippers are capable of forming dimers because they retain a motif of leucines spaced 7 residues apart along an alpha helix. However, leucine zipper moieties of certain embodiments of CLEs provided herein, may or may not retain their DNA binding function.

A spacer of between 1 and 4 alanine residues can be included in CLEs between the extracellular domain that has a dimerizing moiety, and the transmembrane domain. Not to be limited by theory, it is believed that the alanine spacer affects signaling of intracellular domains connected to the leucine zipper extracellular region via the transmembrane domain, by changing the orientation of the intracellular domains.

In illustrative embodiments, CLEs include a cell tag domain. Details regarding cell tags are provided in other sections herein. Any of the cell tags provided herein can be part of a CLE. Typically, the cell tag is linked to the N terminus of the extracellular domain. Not to be limited by theory, in some embodiments, the extracellular domain includes the function of providing a linker, in illustrative embodiments a flexible linker, linking a cell tag domain to a cell that expresses the CLE.

Furthermore, polynucleotides that include a nucleic acid sequence encoding a CLE provided herein, also typically comprise a signal sequence to direct expression to the plasma membrane. Exemplary signal sequences are provided herein in other sections. Elements can be provided on the transcript such that both a CAR and CLE are expressed from the same transcript in certain embodiments.

### Checkpoint Inhibiting Ligands

In some embodiments, RIPs of the present disclosure further comprise nucleic acid sequences encoding a checkpoint-inhibiting ligand. Optionally, the checkpoint-inhibiting ligand is capable of blocking the PD-1/PD-L1 checkpoint. Optionally, the checkpoint-inhibiting ligand is capable of blocking the Tim-3 checkpoint. In some embodiments, such RIPs encode a CAR and/or an LE. In illustrative sub-embodiments the LE is other than an LE that includes an anti-PD-1 ligand. Furthermore, in certain illustrative embodiments, RIPs herein comprise nucleic acid sequences encoding a checkpoint-inhibiting ligand and comprise membrane-bound or membrane-associated cytokine fusion polypeptide. Furthermore, such RIPs can further comprise a membrane-associated activation element, such as a polypeptide that binds CD3.

Checkpoint inhibitor therapy is a form of cancer treatment that uses agents to stimulate or inhibit immune checkpoints and thereby modulate the immune response. Tumors may use checkpoints to protect themselves from the immune system of the subject or from therapeutic agents used in cancer immunotherapy. The present disclosure provides RIPs (e.g., lentiviral particles) comprising a nucleic acid sequence encoding a checkpoint-inhibiting ligand, wherein lentiviral particles produced from the lentiviral vector system display the checkpoint-inhibiting ligand on their surface, and therefore administration of the lentiviral particle results in delivery of the checkpoint-inhibiting ligand to the subject at the site of therapeutic use. The present disclosure further provides lentiviral vector systems comprising a nucleic acid sequence encoding a checkpoint-inhibiting ligand, whereby administration of lentiviral particles delivers the polynucleotide sequence to target cells, which then express the checkpoint-inhibiting ligand at the site of therapeutic use.

Examples of checkpoint-inhibitor ligands provided by the present disclosure include, without limitation, anti-CTLA-4 antibody, anti-PD-1 antibodies, and anti-PD-L1 antibodies or any non-antibody ligands (e.g., nanobodies, DARPins) that interact with CTLA4, PD-1, or PD-L1, respectively. In some embodiments, the checkpoint-inhibiting ligand is capable of blocking the PD-1/PD-L1 checkpoint and/or the Tim-3 checkpoint and/or the CTLA-4 checkpoint.

In some embodiments, any of the RIP formulations or delivery formulations comprising RIPs as disclosed herein for direct administration to a subject can comprise nucleic acid sequences encoding a checkpoint-inhibiting ligand as disclosed herein.

### Anti-Idiotype Polypeptides

Provided herein, in some aspects, are anti-idiotype polypeptides and polynucleotides encoding these polypeptides (as disclosed in detail in other sections herein and in PCT/US21/48532, incorporated by reference herein in its entirety) that have numerous utilities in life sciences and medicine. Such polypeptides, in illustrative embodiments, are especially useful in modified cells, for example for use in cell and gene therapy. Anti-idiotype polypeptides expressed on the surface of cells can recognize target antibodies or target antibody mimetics that come in contact with these cells. These antibodies and antibody mimetics can be used to, for example, mark cells expressing the anti-idiotype polypeptides for killing by the immune system, modulate a property (such as, for example, a proliferative state or an apoptotic state) or activity of the cells, label the cells, provide a target for enrichment and/or purification, enrich the cells, or cause the cells to aggregate. A person skilled in the art will understand how to use the anti-idiotype polypeptides for these and other methods in view of the present disclosure. Accordingly, provided herein are methods for providing any of the above-mentioned uses, by expressing any of the polynucleotides that are disclosed herein and/or in PCT/US21/48532, that include nucleic acids encoding anti-idiotype polypeptides disclosed herein/therein.

Anti-idiotype polypeptides herein, in illustrative aspects, include an extracellular recognition domain (sometimes referred to as an anti-idiotype extracellular recognition domain, anti-id ERD, or anti-Id ECD) and typically include a membrane association domain (MAD), which is separated from the anti-id ECD, in illustrative embodiments, by a stalk. The anti-Id ERD, in illustrative embodiments, includes a recognition domain of an anti-idiotype antibody or anti-idiotype antibody mimetic. The recognition domain of an anti-idiotype polypeptide recognizes the idiotype of a target antibody or the idiotype of a target antibody mimetic. In illustrative embodiments, an anti-idiotype extracellular recognition domain includes an idiotype-binding variable region of an anti-idiotype antibody or anti-idiotype antibody mimetic. In illustrative embodiments, a stalk separates the MAD and the anti-idiotype extracellular recognition domain.

In some embodiments, an extracellular recognition domain recognizes the idiotype of any antibody or antibody mimetic known in the art. In certain illustrative embodiments, the extracellular recognition domain recognizes the idiotype of a clinical antibody or clinical antibody mimetic. Such a clinical antibody, in some illustrative embodiments, is a regulatory agency (e.g., U.S. FDA) approved biologic. In some embodiments, binding of the anti-idiotype polypeptide to the target antibody does not block or prevent binding between the target antibody and its cognate antigen. In illustrative embodiments, binding of the anti-idiotype polypeptide to the target antibody blocks or prevents binding between the target antibody and its cognate antigen.

Typically, anti-idiotype polypeptides include a membrane association domain (sometimes referred to herein as a MAD). The membrane association domain of the anti-idiotype polypeptide attaches, tethers, or anchors the recognition domain from an anti-idiotype antibody or antibody mimetic to a cell membrane. In some embodiments, the membrane association domain comprises one or more of a transmembrane domain and a GPI anchor, as further disclosed elsewhere herein. In some embodiments, the transmembrane domain can be a heterologous transmembrane domain or an endogenous transmembrane domain, either of which could be the transmembrane domain of an antibody.

In some embodiments, anti-idiotype polypeptides provided herein further include one or more intracellular domains (sometimes referred to herein as ICD). Furthermore, the MAD of such anti-idiotype polypeptides that include ICDs in certain illustrative embodiments, is a transmembrane. The one or more intracellular domains can activate or inhibit pro-apoptotic or anti-apoptotic pathways and/or pro-survival or anti-survival pathways, and in certain embodiments modulate other cellular processes/pathways. Details are provided throughout this specification regarding these various embodiments and other embodiments wherein an anti-idiotype polypeptide herein, includes an ICD. In some embodiments, the ICD serves a structural role to assure the anti-id ERD is stably expressed on and remains bound to the cell membrane. In other embodiment an ICD can have functional properties that are regulated by binding dimerization or multimerization that is induced by binding of the anti-id ERD by its target antibody or antibody mimetic.

In some embodiments, the anti-id ERD acts as an inducible extracellular dimerizing domain of an LE herein. In such embodiments, dimerization of an LE by binding of a target antibody to the anti-id ERD can activate signaling domains in the ICD, driving proliferation and/or cell survival. As illustrated, the ICD of an LE can include P3 and optionally P4 domains, as disclosed herein with respect to LE ICDs. In some embodiments, the intracellular domains can activate one or more of a Jak/Stat pathway, a TRAF pathway, a PI3K pathway, or a PLC pathway. Disclosure related to mechanisms for activating these pathways is provided in the “Lymphoproliferative section” herein. Illustrative examples of these embodiments are inducible chimeric lymphoproliferative elements, which are inducible upon binding of a target antibody to an extracellular domain that recognizes the idiotype of a target antibody.

In some embodiments, the anti-id ERD acts as the ASTR of a CAR or TCR. Thus, the CAR or TCR includes the anti-id ERD, a stalk attaching the ERD to a transmembrane domain, and an ICD of a CAR or TCR. Thus, binding of a target antibody to the anti-id ERD can activate signaling domains in the ICD of the CAR or TCR.

In some embodiments, the intracellular domain is pro-apoptotic, and can include one or more intracellular signaling domains from a caspase protein and/or one or more intracellular signaling domains from tumor necrosis factor receptor superfamily members. As such, such embodiments are specific examples of safety switches provided herein. Such embodiments can include an anti-idiotype polypeptide wherein the ICD includes a death domain. Such ICDs can include all or in certain illustrative embodiments, include a portion of an ICD from a TNF receptor superfamily member, that includes a death domain, such as FAS. Illustrative embodiments of such an ant-idiotype polypeptide includes a constitutive dimerization domain in the extracellular domain, that can be all or part of the stalk or transmembrane domain, thus providing such anti-idiotype polypeptide, the ability to form higher order multimers, such as tetramers. Furthermore, ICDs for these embodiments can include death domains, or other functional domains from initiator caspases, such as for example, caspases 2, 8, 9, and 10.

In some embodiments, the anti-id ERD acts as one of the ASTRs of a bi-specific CAR or TCR, wherein a second ASTR is present that typically binds to an antigen expressed by a cancer cell. Such embodiments can be called anti-idiotype bispecific CARs herein. In such embodiments, the CAR or TCR includes the anti-id ERD, a second ASTR, a stalk attaching the anti-id ERD and the 2^{nd} ASTR to a transmembrane domain, and an ICD of a CAR or TCR. Accordingly, binding of a target antibody to the anti-id ERD or binding of the 2^{nd} ASTR to its antigen, can activate signaling domains in the ICD of the CAR or TCR.

In other embodiments of anti-idiotype polypeptides herein that includes an ICD, a cleavage site, typically that is activated by dimerization, is added to the anti-idiotype polypeptide between or as part of the transmembrane domain and the ICD. As such, in some embodiments these anti-idiotype polypeptides include a transmembrane domain that includes an amino acid sequence that serves as a substrate cleavage site upon dimerization, by certain proteases. Such cleavage site can be, in certain embodiments, a substrate cleavage site for gamma-secretase complex, as discussed in more detail herein. The intracellular domain for such embodiments can encode various intracellular polypeptides including certain caspases, including initiator caspases, for example caspases 2, 8, 9, and 10. In other embodiments, the ICD is a transcription factor. In such embodiments, the transcription factor is sequestered outside the nucleus until binding of the anti-id ERD by its target antibody inducing dimerization and cleavage, which releases the transcription factor such that it can enter the nucleus to become active in regulating gene expression.

### Inhibitory RNA Molecules

Embodiments of any of the aspects provided herein can include viral particles, and in illustrative embodiments recombinant retroviral particles, whose genomes are constructed to induce expression of one or more, and in illustrative embodiments two or more, inhibitory RNA molecules, such as for example, a miRNA or shRNA, before or after integration into a host cell, such as a lymphocyte (e.g., a T cell and/or an NK cell). Such inhibitory RNA molecules can be encoded within introns, including for example, an EF1-a intron. Intron sequences that can be used to harbor miRNAs for the present disclosure include any intron that is processed within a T cell. Sequence requirements for introns are known in the art. Such inhibitory RNA molecules can be encoded on genomes of RIPs included in RIP formulations herein.

The inhibitory RNA molecules included in compositions and methods provided herein, in certain illustrative examples, do not exist and/or are not expressed naturally in T cells into whose genome they are inserted. In some embodiments, the inhibitory RNA molecule is a miRNA or an shRNA, or as a precursor of a miRNA, such as for example, a Pri-miRNA or a Pre-miRNA, or a precursor of an shRNA. In some embodiments, the miRNA or shRNA are artificially derived (i.e., artificial miRNAs or siRNAs). In other embodiments, the inhibitory RNA molecule is a dsRNA (either transcribed or artificially introduced) that is processed into an siRNA or the siRNA itself. In some embodiments, the miRNA or shRNA has a sequence that is not found in nature, or has at least one functional segment that is not found in nature, or has a combination of functional segments that are not found in nature.

In some embodiments, inhibitory RNA molecules are positioned in the first nucleic acid molecule in a series or multiplex arrangement such that multiple miRNA sequences are simultaneously expressed from a single polycistronic miRNA transcript. In some embodiments, the inhibitory RNA molecules can be adjoined to one another either directly or indirectly by non-functional linker sequence(s). The linker sequence in some embodiments, is between 5 and 120 nucleotides in length, and in some embodiments can be between 10 and 40 nucleotides in length, as non-limiting examples. In some embodiments, functional sequences can be expressed from the same transcript as the inhibitory RNA molecules, for example, any of the lymphoproliferative elements provided herein. In some embodiments, the inhibitory RNA molecule is a naturally occurring miRNA such as but not limited to miR-155, miR-30, miR-17-92, miR-122, and miR-21. In some embodiments, an inhibitory RNA molecule includes from 5′ to 3′ orientation: a 5′ microRNA flanking sequence, a 5′ stem, a loop, a 3′ stem that is partially or fully complementary to said 5′ stem, and a 3′ microRNA flanking sequence. In certain embodiments, the 5′ microRNA flanking sequence, 3′ microRNA flanking sequence, or both, are derived from a miR-155, such as, e.g., the miR-155 from Mus musculus or Homo sapiens. Inserting a synthetic miRNA stem-loop into a miR-155 framework (i.e., the 5′ microRNA flanking sequence, the 3′ microRNA flanking sequence, and the loop between the miRNA 5′ and 3′ stems) is known to one of ordinary skill in the art (Chung, K. et al. 2006. Nucleic Acids Research. 34(7): e53; U.S. Pat. No. 7,387,896) for example the SIBR and eSIBR sequences. In some embodiments of the present disclosure, miRNAs can be placed in the SIBR or eSIBR miR-155 framework. Any known microRNA framework that is functional to provide proper processing within a cell of miRNAs inserted therein to form mature miRNA capable of inhibiting expression of a target mRNA to which they bind, is contemplated within the present disclosure.

In some embodiments, where two or more inhibitory RNA molecules (in some examples, 1, 2, 3, 4, 5, 6, 7, 8, 9, or 10 inhibitory RNA molecules) are included, these inhibitory RNA molecules are directed against the same or different RNA targets (such as e.g., mRNAs transcribed from genes of interest).

In some embodiments, the one or more inhibitor RNA molecules are one or more lymphoproliferative elements, accordingly, in any aspect or embodiment provided herein that includes a lymphoproliferative element, unless incompatible therewith, or already stated therein.

In some embodiments, the inhibitory RNA, for example miRNA, targets mRNA encoding ABCG1, Cbl Proto-Oncogene (RNF55) (also known as cCBL and RNF55) (HGNC: 1541, Entrez Gene: 867, OMIM: 165360), T-Cell Receptor T3 Zeta Chain (CD3z) (HGNC: 1677, Entrez Gene: 919, OMIM: 186780), T cell receptor alpha locus (TCRA) (also known as TCRα) (HGNC: 12027, Entrez Gene: 6955, OMIM: 186880), T cell receptor beta locus (TCRB) (also known as TCRβ) (HGNC: 12155, Entrez Gene: 6957, OMIM: 186930), PD1, CTLA4, IFN gamma, T Cell Immunoglobulin Mucin 3 (TIM3) (also known as Hepatitis A Virus Cellular Receptor 2) (HGNC: 18437 Entrez Gene: 84868, OMIM: 606652), Lymphocyte Activating 3 (LAG3) (HGNC: 6476, Entrez Gene: 3902, OMIM: 153337), SMAD2, TNF Receptor Superfamily Member 10b (TNFRSF10B) (HGNC: 11905, Entrez Gene: 8795, OMIM: 603612), Protein Phosphatase 2 Catalytic Subunit Alpha (PPP2CA) (HGNC: 9299, Entrez Gene: 5515, OMIM: 176915), Tumor Necrosis Factor Receptor Superfamily Member 6 (TNFRSF6) (also known as Fas Cell Surface Death Receptor (FAS)) (HGNC: 11920, Entrez Gene: 355, OMIM: 134637), B And T Lymphocyte Associated (BTLA) (HGNC: 21087, Entrez Gene: 151888, OMIM: 607925), T Cell Immunoreceptor With Ig And ITIM Domains (TIGIT) (HGNC: 26838, Entrez Gene: 201633, OMIM: 612859), Adenosine A2a Receptor (ADORA2A or A2AR) (HGNC: 263, Entrez Gene: 135, OMIM: 102776), Aryl Hydrocarbon Receptor (AHR) (HGNC: 348, Entrez Gene: 196, OMIM: 600253), Eomesodermin (EOMES) (HGNC: 3372, Entrez Gene: 8320, OMIM: 604615), SMAD Family Member 3 (SMAD3) (HGNC: 6769, Entrez Gene: 4088, OMIM: 603109), SMAD Family Member 4 (SMAD4) (GNC: 6770, Entrez Gene: 4089, OMIM: 600993), TGFBR2, TRAIL2, PP2A, Protein Phosphatase 2 Regulatory Subunit B delta (PPP2R2D) (HGNC: 23732, Entrez Gene: 55844, OMIM: 613992), Tumor Necrosis Factor Ligand Superfamily Member 6 (TNFSF6) (also known as FASL) (HGNC: 11936, Entrez Gene: 356, OMIM: 134638), Caspase 3 (CASP3) HGNC: 1504, Entrez Gene: 836, OMIM: 600636), SOCS1, Suppressor Of Cytokine Signaling 2 (SOCS2) (HGNC: 19382, Entrez Gene: 8835, OMIM: 605117), Kruppel Like Factor 10 (KLF10) (also known as TGFB-Inducible Early Growth Response Protein 1 (TIEG1)) (HGNC: 11810, Entrez Gene: 7071, OMIM: 601878), JunB Proto-Oncogene, AP-1 Transcription Factor Subunit (JunB) (HGNC: 6205, Entrez Gene: 3726, OMIM: 165161), Chromobox 1 (Cbx) (HGNC: 1551, Entrez Gene: 10951, OMIM: 604511), Cbx3, Tet Methylcytosine Dioxygenase 2 (Tet2) (HGNC: 25941, Entrez Gene: 54790, OMIM: 612839), Hexokinase 2 (HK2) (HGNC: 4923, Entrez Gene: 3099, OMIM: 601125), Src homology region 2 domain-containing phosphatase-1 (SHP1) (HGNC: 9658, Entrez Gene: 5777, OMIM: 176883), Src homology region 2 domain-containing phosphatase-2 (SHP2) (HGNC: 9644, Entrez Gene: 5781, OMIM: 176876), colony stimulating factor 2 (CSF2; GMCSF) (Entrez Gene: 1437). In some embodiments, the inhibitory RNA, for example miRNA, targets RNA from a gene that could interfere with beneficial effects of delivering a RIP, or modified T cell or NK cell provided herein: e.g., TCR components that could induce graft versus host disease, HLA components that could induce host versus graft disease, stress ligands, immune checkpoints. Accordingly in some embodiments, the inhibitory RNA, for example miRNA, targets mRNA encoding one or more of a gene, transcribed sequence therein, or coding sequence therein, from one or more of the following: MHC class I gene, a MHC class II gene, a MHC coreceptor gene (e.g., HLA-F, HLA-G), a TCR chain, NKBBiL, LTA, TNF, LTB, LST1, NCR3, AIF1, LY6, a heat shock protein (e.g., HSPA1L, HSPA1A, HSPA1B), complement cascade, regulatory receptors (e.g., NOTCH4), TAP, HLA-DM, HLA-DO, RING1, CD52, CD247, HCP5, DGKA, DGKZ, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD95, CD123, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, SHIP1, SOAT1, SOCS1, T-BET, TET2, TGFBR1, TGFBR2, TGFBR3, TIGIT, TIM3, TOX, and ZFP36L2. In some embodiments, the inhibitory RNA, for example miRNA, targets the antigen that the ASTR of the CAR binds to.

### Synthetic RNA

Provided herein in another aspect, the delivery solution or the cell formulation can include synthetic RNA. In some embodiments the synthetic RNA includes inhibitory RNAs such as siRNAs directed to one or more targets. The targets for these inhibitory RNAs can be any of the targets for siRNAs or miRNAs disclosed elsewhere herein. In some embodiments, the synthetic RNA includes mRNA encoding for one or more proteins or peptides. In some embodiments, the mRNA can encode for any of the genes of interest disclosed elsewhere herein. In some embodiments, the mRNA encodes for one or more cytokines. In some embodiments, the mRNA encodes for one or more CARs. The CARs may be any CAR composition disclosed herein, including bispecific CARs that include an anti-idiotype extracellular recognition domain (also referred to herein as an anti-id ERD) as disclosed herein. In some embodiments, the mRNA can encode any anti-idiotype polypeptide disclosed herein. In some embodiments, the mRNA encodes for the target antibody to an anti-idiotype polypeptide disclosed herein. Such embodiment can have an advantage of providing a target antibody that can be cytotoxic when delivered in soluble form, but less or not cytotoxic when taken up by cells after in vivo administration of an mRNA encoding the target antibody. Thus, cells that take up and express the target antibody can become artificial antigen presenting cells for embodiments where the anti-idiotype polypeptide has an ICD that is the ICD of an LE, or of a CAR (e.g., a bi-specific CAR). In some embodiments, the mRNA encodes one or more proteins or polypeptides that bind and activate a CAR. In some embodiments, the mRNA encodes for an antigen recognized by the ASTR of the CAR. In some embodiments, the mRNA encoding for an antibody recognized by the ASTR of the CAR is an anti-idiotype antibody directed to the antibody or scFv of the ASTR. In some embodiments, the mRNA encodes for an antibody that binds an epitope tag of the CAR and can cross-link two CARs. In some embodiments, the mRNA encodes for one or more T and/or NK cell co-stimulatory proteins. Such co-stimulatory proteins may comprise one or more ligands or antibodies to a co-stimulatory receptor on T and/or NK cells. In some embodiments the co-stimulatory receptor is CD28. In some embodiments the co-stimulatory receptor is 4-1BB. In some embodiments, the mRNA encodes a protein or polypeptide that is soluble. In some embodiments, the mRNA encodes a protein or polypeptide that is membrane-bound. In some embodiments, the membrane-bound protein or polypeptide is operatively linked to a transmembrane domain. In some embodiments, the synthetic RNA includes both inhibitory RNAs such as siRNAs directed to one or more targets and mRNA encoding for one or more proteins or peptides.

In some embodiments, the RNA can be added to a cell formulation comprising, or co-administered with, modified and/or genetically modified T cells and/or NK cells in cell formulations and methods provided herein. In some embodiments, the RNA is added to the isolated blood of a subject and processed in parallel with the T cells and/or NK cells. In some embodiments, the RNA can be formulated separately from the modified and/or genetically modified T cells and/or NK cells. The synthetic RNA may be delivered by any means known in the art for therapeutic delivery of RNA. In some embodiments, the RNA is delivered intravenously. In some embodiments, the RNA is delivered intraperitoneally. In some embodiments, the RNA is delivered intramuscularly. In some embodiments, the RNA is delivered intratumorally. In some embodiments, the RNA is delivered intradermally. In illustrative embodiments, the RNA is delivered subcutaneously. In some embodiments, the RNA is delivered at the same site as the site of administration of the modified and/or genetically modified T cells and/or NK cells. In some embodiments, the RNA is delivered at a site adjacent to the site of administration of the modified and/or genetically modified T cells and/or NK cells. In some embodiments, the RNA is administered once. In some embodiments, the RNA is administered, 2, 3, 4, 5, 6 or more times.

### Packaging Cell Lines/Methods of Making Recombinant Retroviral Particles

The present disclosure provides mammalian packaging cells and packaging cell lines that produce replication incompetent recombinant retroviral particles (RIPs). The cell lines that produce RIPs are also referred to herein as packaging cell lines. A non-limiting example of such method is illustrated in WO2019/055946. RIPs that are produced using methods herein can be used, for example, in RIP formulations provided herein, for example for administering to a subject. Such methods include, for example, a 4 plasmid system or a 5 plasmid system when a nucleic acid encoding an additional membrane bound protein, such as a T cell activation element that is not a fusion with the viral envelope, such as a GPI-linked anti-CD3, is included (See WO2019/05546). In an illustrative embodiment, provided herein is a 4 plasmid system in which a T cell activation element, such as a GPI-linked anti-CD3, is encoded on one of the packaging plasmids such as the plasmid encoding the viral envelope protein or the plasmid encoding REV, and optionally a second viral membrane-associated transgene such as a membrane bound cytokine can be encoded on the other packaging plasmid. In each case the nucleic acid encoding the viral protein is separated from the transgene by an IRES or a ribosomal skip sequence such as P2A or T2A. Such 4 plasmid system and associated polynucleotides as stated in the Examples, provided increased titers as compared to a 5 vector system in transient transfections, and thus provide illustrative embodiments herein. The present disclosure provides packaging cells and mammalian cell lines that are packaging cell lines that produce replication incompetent recombinant retroviral particles that genetically modify target mammalian cells and the target mammalian cells themselves. In illustrative embodiments, the packaging cell comprises nucleic acid sequences encoding a packageable RNA genome of the replication incompetent retroviral particle, a REV protein, a gag polypeptide, a pol polypeptide, and a pseudotyping element.

The cells of the packaging cell line can be adherent or suspension cells. Exemplary cell types are provided hereinbelow. In illustrative embodiments, the packaging cell line can be a suspension cell line, i.e., a cell line that does not adhere to a surface during growth. The cells can be grown in a chemically-defined media and/or a serum-free media. In some embodiments, the packaging cell line can be a suspension cell line derived from an adherent cell line, for example, the HEK293 cell line can be grown in conditions to generate a suspension-adapted HEK293 cell line according to methods known in the art. The packaging cell line is typically grown in a chemically defined media. In some embodiments, the packaging cell line media can include serum. In some embodiments, the packaging cell line media can include a serum replacement, as known in the art. In illustrative embodiments, the packaging cell line media can be serum-free media. Such media can be a chemically defined, serum-free formulation manufactured in compliance with Current Good Manufacturing Practice (CGMP) regulations of the US Food and Drug Administration (FDA). The packaging cell line media can be xeno-free and complete. In some embodiments, the packaging cell line media has been cleared by regulatory agencies for use in ex vivo cell processing, such as an FDA 510(k) cleared device.

Accordingly, in one aspect, provided herein is a method of making a replication incompetent recombinant retroviral particle including: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; and B. harvesting the replication incompetent recombinant retroviral particle from the serum-free media. In another aspect, provided herein is a method of transducing a lymphocyte with a replication incompetent recombinant retroviral particle comprising: A. culturing a packaging cell in suspension in serum-free media, wherein the packaging cell comprises nucleic acid sequences encoding a packageable RNA genome of the replication incompetent retroviral particle, a REV protein, a gag polypeptide, a pol polypeptide, and a pseudotyping element; B. harvesting the replication incompetent recombinant retroviral particle from the serum-free media; and C. contacting the lymphocyte with the replication incompetent recombinant retroviral particle, wherein the contacting is performed for less than 24 hours, 20 hours, 18 hours, 12 hours, 8 hours, 4 hours, 2 hours, 1 hour, 30 minutes, or 15 minutes (or between contacting and no incubation, or 15 minutes, 30 minutes, 1, 2, 3, or 4 hours on the low end of the range and 1, 2, 3, 4, 6, 8, 12, 18, 20, or 24 hours on the high end of the range), thereby transducing the lymphocyte.

The packageable RNA genome, in certain illustrative embodiments, is designed to express one or more target polypeptides, including as a non-limiting example, any of the engineered signaling polypeptides disclosed herein and/or one or more (e.g., two or more) inhibitory RNA molecules in opposite orientation (e.g., encoding on the opposite strand and in the opposite orientation), from retroviral components such as gag and pol. For example, the packageable RNA genome can include from 5′ to 3′: a 5′ long terminal repeat, or active truncated fragment thereof; a nucleic acid sequence encoding a retroviral cis-acting RNA packaging element; a nucleic acid sequence encoding a first and optionally second target polypeptide, such as, but not limited to, an engineered signaling polypeptide(s) in opposite orientation, which can be driven off a promoter in this opposite orientation with respect to the 5′ long terminal repeat and the cis-acting RNA packaging element, which in some embodiments is called a “fourth” promoter for convenience only (and sometimes referred to herein as the promoter active in T cells and/or NK cells), which is active in a target cell such as a T cell and/or an NK cell but in illustrative examples is not active in the packaging cell or is only inducibly or minimally active in the packaging cell; and a 3′ long terminal repeat, or active truncated fragment thereof. In some embodiments, the packageable RNA genome can include a central polypurine tract (cPPT)/central termination sequence (CTS) element. In some embodiments, the retroviral cis-acting RNA packaging element can be HIV Psi. In some embodiments, the retroviral cis-acting RNA packaging element can be the Rev Response Element. The engineered signaling polypeptide driven by the promoter in the opposite orientation from the 5′ long terminal repeat, in illustrative embodiments, is one or more of the engineered signaling polypeptides disclosed herein and can optionally express one or more inhibitory RNA molecules as disclosed in more detail herein and in WO2017/165245A2, WO2018/009923A1, and WO2018/161064A1.

It will be understood that promoter number, such as a first, second, third, fourth, etc. promoter is for convenience only. A promoter that is called a “fourth” promoter should not be taken to imply that there are any additional promoters, such as first, second or third promoters, unless such other promoters are explicitly recited. It should be noted that each of the promoters are capable of driving expression of a transcript in an appropriate cell type and such transcript forms a transcription unit.

In some embodiments, the engineered signaling polypeptide can include a first lymphoproliferative element. Suitable lymphoproliferative elements are disclosed in other sections herein. As a non-limiting example, the lymphoproliferative element can be expressed as a fusion with a cell tag, such as an eTag, as disclosed herein. In some embodiments, the packageable RNA genome can further include a nucleic acid sequence encoding a second engineered polypeptide including a chimeric antigen receptor, encoding any CAR embodiment provided herein. For example, the second engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments where the target cell is a T cell, the promoter that is active in a target cell is active in a T cell, as disclosed elsewhere herein.

In some embodiments, the engineered signaling polypeptide can include a CAR, and the nucleic acid sequence can encode any CAR embodiment provided herein. For example, the engineered polypeptide can include a first antigen-specific targeting region, a first transmembrane domain, and a first intracellular activating domain. Examples of antigen-specific targeting regions, transmembrane domains, and intracellular activating domains are disclosed elsewhere herein. In some embodiments, the packageable RNA genome can further include a nucleic acid sequence encoding a second engineered polypeptide. In some embodiments, the second engineered polypeptide can be a lymphoproliferative element. In some embodiments where the target cell is a T cell or NK cell, the promoter that is active in a target cell is active in a T cell or NK cell, as disclosed elsewhere herein.

In some embodiments, the packageable RNA genome included in any of the aspects provided herein, can further include a riboswitch, as discussed in WO2017/165245A2, WO2018/009923A1, and WO2018/161064A1. In some embodiments, the nucleic acid sequence encoding the engineered signaling polypeptide can be in a reverse orientation with respect to the 5′ to 3′ orientation established by the 5′ LTR and the 3′ LTR. In further embodiments, the packageable RNA genome can further include a riboswitch and, optionally, the riboswitch can be in reverse orientation. In any of the embodiments disclosed herein, a polynucleotide including any of the elements can include a primer binding site. In illustrative embodiments, insulators and/or polyadenylation sequences can be placed before, after, between, or near genes to prevent or reduce unregulated transcription. In some embodiments, the insulator can be chicken HS4 insulator, Kaiso insulator, SAR/MAR elements, chimeric chicken insulator-SAR elements, CTCF insulator, the gypsy insulator, or the p3-globin insulator or fragments thereof known in the art. In some embodiments, the insulator and/or polyadenylation sequence can be hGH polyA (SEQ ID NO:316), SPA1 (SEQ ID NO:317), SPA2 (SEQ ID NO:318), b-globin polyA spacer B (SEQ ID NO:319), b-globin polyA spacer A (SEQ ID NO:320), 250 cHS4 insulator v1 (SEQ ID NO:321), 250 cHS4 insulator v2 (SEQ ID NO:322), 650 cHS4 insulator (SEQ ID NO:323), 400 cHS4 insulator (SEQ ID NO:324), 650 cHS4 insulator and b-globin polyA spacer B (SEQ ID NO:325), or b-globin polyA spacer B and 650 cHS4 insulator (SEQ ID NO:326).

In any of the embodiments disclosed herein, a nucleic acid sequence encoding Vpx can be on the second or an optional third transcriptional unit, or on an additional transcriptional unit that is operably linked to the first inducible promoter.

Some aspects of the present disclosure include or are cells, in illustrative examples, mammalian cells, that are used as packaging cells to make replication incompetent recombinant retroviral particles, such as lentiviruses, for transduction of T cells and/or NK cells.

Any of a wide variety of cells can be selected for in vitro production of a virus or virus particle, such as a redirected recombinant retroviral particle, according to the invention. Eukaryotic cells are typically used, particularly mammalian cells including human, simian, canine, feline, equine and rodent cells. In illustrative examples, the cells are human cells. In further illustrative embodiments, the cells reproduce indefinitely, and are therefore immortal. Examples of cells that can be advantageously used in the present invention include NIH 3T3 cells, COS cells, Madin-Darby canine kidney cells, human embryonic 293T cells and any cells derived from such cells, such as gpnlslacZ pNX cells, which are derived from 293T cells. Highly transfectable cells, such as human embryonic kidney 293T cells, can be used. By “highly transfectable” it is meant that at least about 50%, more preferably at least about 70% and most preferably at least about 80% of the cells can express the genes of the introduced DNA.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), C127, A549, RATl cells, mouse L cells (ATCC No. CCLI.3), mouse myeloma, human embryonic kidney (HEK) cells (ATCC No. CRL1573), HEK-293, HEK-293T, HEK-293E, HEK-293 FT, HEK-293S, HEK-293SG, HEK-293 FTM, HEK-293SGGD, HEK-293A, 293RTV, GP2-293, MDCK, HLHepG2 cells, Hut-78, Jurkat, HL-60, PerC6, 91-1, and the like.

### Nucleic Acids

The present disclosure provides nucleic acid encoding polypeptides of the present disclosure and nucleic acids are disclosed for use in various methods herein. A nucleic acid will in some embodiments be DNA, including, e.g., a recombinant expression construct, or as all or part of the genome of a T cell or an NK cell, for example. A nucleic acid will in some embodiments be RNA, such as a retroviral genome (for example included in RIPs provided hererin) or an expressed transcript within a packaging cell line, a T cell or an NK, for example. A nucleic acid will in some embodiments be RNA, e.g., in vitro synthesized RNA. In some embodiments, the nucleic acid can be isolated. As used herein, the term “isolated” means that the material is removed from its original environment (e.g., the natural environment if it is naturally occurring). For example, a naturally-occurring polynucleotide, or in other embodiments a polypeptide, present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment. For example, an isolated nucleic can be part of recombinant nucleic acid vector, such as an expression vector, which in illustrative embodiments can be a replication incompetent recombinant retroviral particle. In some embodiments, the nucleic acid is manufactured in compliance with cGMP, as discussed herein for kit components.

In some embodiments, a nucleic acid provides for production of a polypeptide of the present disclosure, e.g., in a mammalian cell. In other cases, a subject nucleic acid provides for amplification of the nucleic acid encoding a polypeptide of the present disclosure.

For packaging viral particles, promoters suitable for use may be constitutive or inducible. For expression of the viral particle RNA, an LTR promoter or hybrid LTR promoter may be used. For example, RSV/LTR, TRE/LTR or LTR alone may be used to transcribe the nucleic acid to be packaged. Examples of LTR's include, but are not limited to, MSCV, GALV, HIV-1, HIV-2 and MuLV. In packaging lines containing the large T antigen, for example, incorporation of the SV40 origin of replication may be included in one or more packaging vectors to amplify circular plasmid DNA during transcription and/or translation. The use of multiple promoters may be utilized to prevent transcription factor competition. For example, CMV, SV40, RSV, HSVTK, TRE and other promoters may be utilized to express different components of the LV particles. In some instances, viral particle components may be expressed from integrated expression vectors. In other cases, one or more of the nucleic acids may be introduced via transient expression. In some embodiments the use of inducible promoters are used to minimize cellular toxicity before viral particle packaging.

For expression of a transgene (e.g., a CAR) in a genetically modified cell, such as a lymphocyte, a macrophage, or a dendritic cell, suitable promoters include any constitutive promoter known in the art. In some embodiments, the constitutive promoter can be an EF1-a promoter, PGK promoter, CMV promoter, MSCV-U3 promoter (see, e.g., Jones et al., Human Gene Therapy (2009) 20: 630-40), SV40hCD43 promoter, VAV promoter, TCRβpromoter, UBC promoter, cytomegalovirus immediate early promoter, herpes simplex virus thymidine kinase promoter, early and late SV40 promoters, promoter present in long terminal repeats from a retrovirus, mouse metallothionein-I promoter, and various art-known tissue specific promoters. In some embodiments, a constitutive promoter can include the EF1-a promoter nucleotide sequence (SEQ ID NO:350), the PGK promoter nucleotide sequence (SEQ ID NO:351), or a functional portion or variant thereof. In some embodiments, a constitutive promoter can include other than the EF1-a promoter. In some embodiments, the promoter include light and/or heavy chain immunoglobulin gene promoter and enhancer elements.

In some embodiments, the promoter is not active in the packaging line or is only minimally active in the packaging line. Such embodiments have the advantage with expression an engineered T cell receptor or a CAR, that they would reduce, minimize, or in illustrative embodiments substantially eliminate, or even eliminate expression of the engineered T cell receptor or CAR in a encapsulated nucleic acid vector such as a RIR retroviral particle or a virus-like particle because of reduced, low, negligible, substantially no, or no expression of the engineered T cell receptor or CAR in the packaging cell line used to make the encapsulated nucleic acid vector. In illustrative embodiments, such expression is reduced, substantially eliminated, or eliminated on the surface of the encapsulated nucleic acid vector (e.g., RIR particle or virus-like particle). In some embodiments, the promoter can be a T cell-specific promoter, a CD8 cell-specific promoter, a CD4 cell-specific promoter, a NKT cell specific promoter, or an NK cell-specific promoter. In some embodiments, the T cell-specific promoter can be the CD3 zeta promoter or the CD3 delta promoter (see, e.g., Ji et al., J Biol Chem. 2002 Dec. 6; 277(49):47898-906). In illustrative embodiments, the T cell-specific promoter can be the CD3 zeta promoter. In some embodiments, the T cell-specific promoter can be a CD8 gene promoter. In some embodiments, the T cell-specific promoter can be a CD4 gene promoter (see, e.g., Salmon et al. (1993) Proc. Natl. Acad. Sci. USA 90:7739; and Marodon et al. (2003) Blood 101:3416). In some embodiments, an NK cell-specific promoter can be a Neri (p46) promoter (see, e.g., Eckelhart et al. (2011) Blood 117:1565). In some embodiments, the specific proteins encoded by the recombinant gene vector are not expressed, displayed, and/or incorporated in the surface of the gene vector (e.g., RIP). In some embodiments, this is accomplished by means of a T cell specific promoter driving transgene expression in the gene vector. In some embodiments, that promoter is a promoter from the CD3 family. In other embodiments it is a hybrid CD3 promoter. In other embodiments, the packaging cell line encodes a repressor protein capable of substantially suppressing the expression of the lentiviral transgenes in the packaging cell line. In some embodiments that suppressor may be a TET repressor protein. In yet other embodiments, the transcription factor activate against the protein in the packaging line has been suppressed or inactivated. In some embodiments the inactivation may be achieved through DNA editing nucleases. In other embodiments the inactivation is achieved through shRNA or miRNA. In other embodiments, the suppression of the transcription factor is achieved through a dominant negative protein or degron to the transcription factor. In other embodiments, the viral nucleic acids are controlled via a ligand inducible or repressible promoter not activated in the packaging cell line. In other embodiments, the promoter can be a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like. Further discussion of suitable promoters for use in various methods and as separate aspects, are provided herein.

In some embodiments, the promoter is inducible in a cell to be genetically modified (e.g., CAR-T cell). In some embodiments, an inducible promoter can include a T cell-specific response element or an NFAT response element. In other embodiments the promoter may be regulated by an environmental condition, such as hypoxia, temperature, glucose, pH or light. In other embodiments the promoter may be responsive to concentrations of extracellular molecules. In some instances, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28: e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, MA), the disclosures of which are incorporated herein by reference.

In some embodiments, the promoter is a CD8 cell-specific promoter, a CD4 cell-specific promoter, a macrophage-specific promoter, or an NK-specific promoter. For example, a CD4 gene promoter can be used.

In some embodiments, e.g., for expression in a yeast cell, a suitable promoter is a constitutive promoter such as an ADH1, PGKI, ENO, or PYKI promoter and the like; or a regulatable promoter such as a GALI, GALlO, ADH2, PH05, CUPI, GAL7, MET25, MET3, CYCI, HIS3, ADH1, PGK, GAPDH, ADCI, TRP1, URA3, LEU2, ENO, TPl, or AOXl promoter (e.g., for use in Pichia). Selection of the appropriate vector and promoter is well within the level of ordinary skill in the art.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), apagC promoter (Pulkkinen and Miller, J. Bacterial., 1991: 173(1): 86-93; Alpuche-Aranda et al., PNAS, 1992; 89(21): 10079-83), a nirB promoter (Harborne et al. (1992) Mal. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mal. Microbial. 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Laci repressor protein changes conformation when contacted with lactose, thereby preventing the Laci repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

An isolated nucleotide sequence encoding a polypeptide of the disclosure can be present in a eukaryotic expression vector and/or a cloning vector. Nucleotide sequences encoding two separate polypeptides can be cloned in the same or separate vectors. An expression vector can include a selectable marker, an origin of replication, and other features that provide for replication and/or maintenance of the vector and expression of a transgene. For example, an expression vector typically includes a promoter operably linked to a transgene. Suitable expression vectors are known in the art and include, for example, plasmids and viral vectors. In some embodiments, the expression vector is a recombinant retroviral particle, as disclosed in detail herein.

Large numbers of suitable vectors and promoters are known to those of skill in the art; many are commercially available for generating subject recombinant constructs. The following bacterial vectors are provided by way of example: pBs, phagescript, PsiX174, pBluescript SK, pBs KS, pNH8a, pNH16a, pNH18a, pNH46a (Stratagene, La Jolla, CA, USA); pTrc99A, pKK223-3, pKK233-3, pDR540, and pRIT5 (Pharmacia, Uppsala, Sweden). The following eukaryotic vectors are provided by way of example: pWLneo, pSV2cat, pOG44, PXR1, pSG (Stratagene) pSVK3, pBPV, pMSG, and pSVL (Pharmacia).

Expression vectors generally have convenient restriction sites located near the promoter sequence to provide for the insertion of nucleic acid sequences encoding heterologous proteins. A selectable marker operative in the expression host may be present.

As noted above, in some embodiments, a nucleic acid encoding a polypeptide of the present disclosure will in some embodiments be RNA, e.g., in vitro synthesized RNA. Methods for in vitro synthesis of RNA are known in the art; any known method can be used to synthesize RNA including a nucleotide sequence encoding a polypeptide of the present disclosure. Methods for introducing RNA into a host cell are known in the art. See, e.g., Zhao et al. (2010) Cancer Res. 15:9053. Introducing RNA including a nucleotide sequence encoding a polypeptide of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

Various aspects and embodiments that include a polynucleotide, a nucleic acid sequence, and/or a transcriptional unit, and/or a vector including the same, further include one or more of a Kozak-type sequence (also called a Kozak-related sequence herein), a woodchuck hepatitis virus post-transcriptional regulatory element (WPRE), and a double stop codon or a triple stop codon, wherein one or more stop codons of the double stop codon or the triple stop codon define a termination of a reading from of at least one of the one or more transcriptional units. In certain embodiments, a polynucleotide a nucleic acid sequence, and/or a transcriptional unit, and/or a vector including the same, further includes a Kozak-type sequence having a 5′ nucleotide within 10 nucleotides upstream of a start codon of at least one of the one or more transcriptional units. Kozak determined the Kozak consensus sequence, (GCC)GCCRCCATG (SEQ ID NO:327), for 699 vertebrate mRNAs, where R is a purine (A or G) (Kozak. Nucleic Acids Res. 1987 Oct. 26; 15(20):8125-48). In one embodiment the Kozak-type sequence is or includes CCACCAT/UG(G) (SEQ ID NO:328), CCGCCAT/UG(G) (SEQ ID NO:329), GCCGCCGCCAT/UG(G) (SEQ ID NO:330), or GCCGCCACCAT/UG(G) (SEQ ID NO:331) (with nucleotides in parenthesis representing optional nucleotides and nucleotides separated by a slash indicated different possible nucleotides at that position, for example depending on whether the nucleic acid is DNA or RNA. In these embodiments that include the AU/TG start codon, the A can be considered position 0. In certain illustrative embodiments, the nucleotides at −3 and +4 are identical, for example the −3 and +4 nucleotides can be G. In another embodiment the Kozak-type sequence includes an A or G in the 3rd position upstream of ATG where ATG is the start codon. In another embodiment the Kozak-type sequence includes an A or G in the 3rd position upstream of AUG where AUG is the start codon. In an illustrative embodiment, the Kozak sequence is (GCC)GCCRCCATG (SEQ ID NO:327), where R is a purine (A or G). In an illustrative embodiment, the Kozak-type sequence is GCCGCCACCAUG (SEQ ID NO:332). In another embodiment, which can be combined with the preceding embodiment that includes a Kozak-type sequence and/or the following embodiment that includes triple stop codon, the polynucleotide includes a WPRE element. WPREs have been characterized in the art (See e.g., (Higashimoto et al., Gene Ther. 2007; 14: 1298)) and as illustrated in WO2019/055946. In some embodiments, the WPRE element is located 3′ of a stop codon of the one or more transcriptional units and 5′ to a 3′ LTR of the polynucleotide. In another embodiment, which can be combined with either or both of the preceding embodiments (i.e., an embodiment wherein the polynucleotide includes a Kozak-type sequence and/or an embodiment wherein the polynucleotide includes a WPRE), the one or more transcriptional units terminates with one or more stop codons of a double stop codon or a triple stop codon, wherein the double stop codon includes a first stop codon in a first reading frame and a second stop codon in a second reading frame, or a first stop codon in frame with a second stop codon, and wherein the triple stop codon includes a first stop codon in a first reading frame, a second stop codon in a second reading frame, and a third stop codon in a third reading frame, or a first stop codon in frame with a second stop codon and a third stop codon.

A triple stop codon herein includes three stop codons, one in each reading frame, within 10 nucleotides of each other, and preferably having overlapping sequence, or three stop codons in the same reading frame, preferably at consecutive codons. A double stop codon means two stop codons, each in a different reading frame, within 10 nucleotides of each other, and preferably having overlapping sequences, or two stop codons in the same reading frame, preferably at consecutive codons.

In some of the methods and compositions disclosed herein, the introduction of DNA into PBMCs, B cells, T cells and/or NK cells and optionally the incorporation of the DNA into the host cell genome, is performed using methods that use recombinant nucleic acid vectors other than replication incompetent recombinant retroviral particles. For example, other viral vectors can be utilized, such as those derived from adenovirus, adeno-associated virus, or herpes simplex virus-1, as non-limiting examples.

In some embodiments, methods provided herein, and associated uses, reaction mixtures, kits and cell formulations can include transfecting cells with polynucleotides that are not encoded in viral vectors. Such polynucleotides can be referred to as non-viral vectors. In any of the embodiments disclosed herein that utilize non-viral vectors to genetically modify or transfect cells, the non-viral vectors, including for example, plasmids or naked DNA, can be introduced into the cells, such as for example, PBMCs, B cells, T cells and/or NK cells using methods that include electroporation, nucleofection, liposomal formulations, lipids, dendrimers, cationic polymers such as poly(ethylenimine) (PEI) and poly(l-lysine) (PLL), nanoparticles, cell-penetrating peptides, microinjection, and/or non-integrating lentiviral vectors. In some embodiments, the liposomal formulations, lipids, dendrimers, PEI, PLL, nanoparticles, and cell-penetrating peptides can be modified to include lymphocyte-targeting ligands, for example, an anti-CD3 antibody. PEI coupled to anti-CD3 antibodies was shown to efficiently transfect PBMCs with an exogenous nucleic acid (O'Neill et al. Gene Ther. 2001 March; 8(5):362-8). Similarly, nanoparticles made from polyglutamic acid molecules coupled to anti-CD3e f(ab′)2 fragments transfected T lymphocytes (Smith et al. Nat Nanotechnol. 2017 August; 12(8): 813-820). In some embodiments, DNA can be introduced into cells, such as PBMCs, B cells, T cells and/or NK cells in a complex with liposomes and protamine. Other methods for transfecting T cells and/or NK cells ex vivo that can be used in embodiments of methods provided herein, are known in the art (see e.g., Morgan and Boyerinas, Biomedicines. 2016 Apr. 20; 4(2). pii: E9, incorporated by reference herein in its entirety).

In some embodiments of method provided herein, DNA can be integrated into the genome using transposon-based carrier systems by co-transfection, co-nucleofection or co-electroporation of target DNA as plasmid containing the transposon ITR fragments in 5′ and 3′ ends of the gene of interest and transposase carrier system as DNA or mRNA or protein or site specific serine recombinases such as phiC31 that integrates the gene of interest in pseudo attP sites in the human genome, in this instance the DNA vector contains a 34 to 40 bp attB site that is the recognition sequence for the recombinase enzyme (Bhaskar Thyagarajan et al. Site-Specific Genomic Integration in Mammalian Cells Mediated by Phage pC31 Integrase, Mol Cell Biol. 2001 June; 21(12): 3926-3934) and co transfected with the recombinase. For T cells and/or NK cells, transposon-based systems that can be used in certain methods provided herein utilize the Sleeping Beauty DNA carrier system (see e.g., U.S. Pat. No. 6,489,458 and U.S. patent application Ser. No. 15/434,595, incorporated by reference herein in their entireties), the PiggyBac DNA carrier system (see e.g., Manuri et al., Hum Gene Ther. 2010 April; 21(4):427-37, incorporated by reference herein in its entirety), or the ToLCDR2transposon system (see e.g., Tsukahara et al., Gene Ther. 2015 February; 22(2): 209-215, incorporated by reference herein in its entirety) in DNA, mRNA, or protein form. In some embodiments, the transposon and/or transposase of the transposon-based vector systems can be produced as a minicircle DNA vector before introduction into T cells and/or NK cells (see e.g., Hudecek et al., Recent Results Cancer Res. 2016; 209:37-50 and Monjezi et al., Leukemia. 2017 January; 31(1):186-194, incorporated by reference herein in their entireties). However, in some situations, the transposase-based carrier systems are not the preferred method of introducing an exogenous nucleic acid. Thus, in some embodiments, a polynucleotide of any of the aspects or embodiments disclosed herein does not include the transposon ITR fragments. In some embodiments, a modified, genetically modified, and/or transduced cell of any of the aspects or embodiments disclosed herein does not include the transposase carrier system as DNA or mRNA or protein.

The CAR or lymphoproliferative element can also be integrated into the defined and specific sites in the genome using CRISPR or TALEN mediated integration, by adding 50-1000 bp homology arms homologous to the integration 5′ and 3′ of the target site (Jae Seong Lee et al. Scientific Reports 5, Article number: 8572 (2015), Site-specific integration in CHO cells mediated by CRISPR/Cas9 and homology-directed DNA repair pathway). CRISPR or TALEN provide specificity and genomic-targeted cleavage and the construct will be integrated via homology-mediated end joining (Yao X et al. Cell Res. 2017 June; 27(6):801-814. doi: 10.1038/cr.2017.76. Epub 2017 May 19). The CRISPR or TALEN can be co-transfected with target plasmid as DNA, mRNA, or protein.

For any of the methods for modifying, genetically modifying, and/or transducing T and/or NK cells (e.g., in whole blood or in whole blood fractions such as TNCs or PBMCs), or uses that include such methods, or modified cells produced using such methods, and any other method or product-by-process provided herein, a skilled artisan will understand where an exogenous nucleic acid(s) could be introduced into the cells using methods that do not include a replication incompetent recombinant retroviral particle, for example using another type of recombinant vector (e.g., a plasmid associated with a lipid transfection agent).

### Polypeptide Variants

In any of the embodiments disclosed herein, the amino acids in a polypeptide sequences can contain substitutions or variations. The polypeptides obtained by performing substitutions or variations in the amino acid sequences can be referred to as polypeptide variants or variants, for example, a chimeric antigen receptor variant or a lymphoproliferative element variant. In some embodiments, the polypeptides disclosed herein can be polypeptide variants. Variants, can be prepared by introducing appropriate changes into the nucleotide sequence of the nucleic acids encoding the polypeptide. Alternatively, variants can be prepared by peptide synthesis. Such modifications include, for example, deletions, insertions, and/or substitutions of residues within the amino acid sequences of the polypeptide. Any combination of deletion, insertion, and substitution can be made to generate the variant, provided that the variant possesses the desired characteristics, for example, lymphoproliferative activity, or antigen binding. Not to be limited by theory, variations can be introduced into an antibody or antibody mimetic to improve the binding affinity, and/or other biological properties of the antibody or antibody mimetic. In some embodiments, the variants include one or more amino acid substitutions.

In some embodiments, an alanine (Ala) residue can be substituted with valine (Val), leucine (Leu), or isoleucine (Ile). In some embodiments, an arginine (Arg) residue can be substituted with lysine (Lys), glutamine (Gln), or asparagine (Asn). In some embodiments, an asparagine (Asn) residue can be substituted with glutamine (Gln), histidine (His), aspartic acid (Asp), lysine (Lys), or arginine (Arg). In some embodiments, an aspartic acid (Asp) residue can be substituted with glutamic acid (Glu) or asparagine (Asn). In some embodiments, a cysteine (Cys) residue can be substituted with serine (Ser) or alanine (Ala). In some embodiments, a glutamine (Gln) residue can be substituted with asparagine (Asn) or glutamic acid (Glu). In some embodiments, a glutamic acid (Glu) residue can be substituted with aspartic acid (Asp) or glutamine (Gln). In some embodiments, a glycine (Gly) residue can be substituted with alanine (Ala). In some embodiments, a histidine (His) residue can be substituted with asparagine (Asn), glutamine (Gln), lysine (Lys), or arginine (Arg). In some embodiments, an isoleucine (Ile) residue can be substituted with leucine (Leu), valine (Val), methionine (Met), alanine (Ala), phenylalanine (Phe), or norleucine. In some embodiments, a leucine (Leu) residue can be substituted with norleucine, isoleucine (Ile), valine (Val), methionine (Met), alanine (Ala), or phenylalanine (Phe). In some embodiments, a lysine (Lys) residue can be substituted with arginine (Arg), glutamine (Gln), or asparagine (Asn). In some embodiments, a methionine (Met) residue can be substituted with leucine (Leu), phenylalanine (Phe), or isoleucine (Ile). In some embodiments, a phenylalanine (Phe) residue can be substituted with tryptophan (Trp), leucine (Leu), valine (Val), isoleucine (Ile), alanine (Ala), or tyrosine (Tyr). In some embodiments, a proline residue can be substituted with alanine (Ala). In some embodiments, a serine (Ser) residue can be substituted with threonine (Thr). In some embodiments, a threonine (Thr) residue can be substituted with valine (Val) or serine (Ser). In some embodiments, a tryptophan (Trp) can be substituted with tyrosine (Tyr) or phenylalanine (Phe). In some embodiments, a tyrosine (Tyr) residue can be substituted with tryptophan (Trp), phenylalanine (Phe), threonine (Thr), or senne (Ser). In some embodiments, a valine (Val) residue can be substituted with isoleucine (Ile), leucine (Leu), methionine (Met), phenylalanine (Phe), alanine (Ala), or norleucine.

A skilled artisan will understand that any of the embodiments disclosed herein that include polypeptides can instead include a polypeptide variant as disclosed in this section.

### Characterization and Commercial Production Methods

The present disclosure provides various methods and compositions that can be used as research reagents in scientific experimentation and for commercial production. Such scientific experimentation can include methods for characterization of lymphocytes, such as NK cells and in illustrative embodiments, T cells using methods for administering RIPs to a subject, or methods for modifying, for example genetically modifying and/or transducing lymphocytes provided herein. Such methods for example, can be used to study activation of lymphocytes and the detailed molecular mechanisms by which activation makes such cells transducible. Furthermore, provided herein are modified and in illustrative embodiments genetically modified lymphocytes that will have utility for example, as research tools to better understand factors that influence T cell proliferation and survival. Such modified lymphocytes, such as NK cells and in illustrative embodiments T cells, can furthermore be used for commercial production, for example for the production of certain factors, such as growth factors and immunomodulatory agents, that can be harvested and tested or used in the production of commercial products.

The scientific experiments and/or the characterization of lymphocytes can include any of the aspects, embodiments, or subembodiments provided herein useful for analyzing or comparing lymphocytes. In some embodiments, T cells and/or NK cells can be transduced with the RIPs provided herein that include polynucleotides. In some embodiments, transduction of the T cells and/or NK cells can include polynucleotides that include polynucleotides encoding polypeptides of the present disclosure, for example, CARs, lymphoproliferative elements, and/or activation elements. In some embodiments, the polynucleotides can include inhibitory RNA molecules as discussed elsewhere herein. In some embodiments, the lymphoproliferative elements can be chimeric lymphoproliferative elements.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein and all of the possible combinations of the disclosed aspects and embodiments are not listed in this section. Additional embodiments and aspects are provided in other sections herein. Furthermore, it will be understood that embodiments are provided that are specific embodiments for many aspects, as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are discussed more specifically in other sections of this detailed description. Thus, for example any of the embodiments provided herein can be used in any of the reaction mixture, cell formulation, kit, use, cell processing assembly, filter assembly, cell population, modified, genetically modified, and transduced T cell or NK cell, mixtures, cell mixtures, or method aspect provided herein, unless incompatible with, or otherwise stated.

Provided herein are methods, uses, compositions, and kits that include a hyaluronidase polypeptide, including for example retroviral particles that encode a hyaluronidase polypeptide or that include a membrane-bound hyaluronidase polypeptide. Such hyaluronidase polypeptide in some embodiments is a novel, chimeric hyaluronidase provided herein. Accordingly, provided herein in one aspect is a replication incompetent recombinant retroviral particle (RIP), comprising a viral envelope, a nucleic acid encoding a packaging signal, Gag, and a membrane-bound hyaluronidase. Provided herein in another aspect is a replication incompetent recombinant retroviral particle (RIP) formulation, delivery solution, delivery reagent, or composition (e.g. pharmaceutical composition) comprising a plurality of RIPs, wherein each of the RIPs comprises a viral envelope, a nucleic acid encoding a packaging element, Gag, and a membrane-bound hyaluronidase polypeptide. Another aspect herein includes a method of administering to a subject, that includes administering any of these RIP, RIP formulations, delivery reagents, or compositions that include a membrane-bound hyaluronidase, to the subject. In some embodiments, the method of administering is a method of treating a disease or disorder.

In some aspects, provided herein are isolated hyaluronidase polypeptides, and nucleic acids encoding the same. Such isolated hyaluronidase polypeptides include, for example, chimeric hyaluronidase polypeptides as provided herein.

In any of the aspects and embodiments herein that include a viral particle, for example a RIP, the viral particle, for example RIP, further includes a polynucleotide that is a gene of interest (GOI), or that comprises one or more transcriptional units, wherein each of the one or more transcriptional units is operatively linked to a promoter active in a target cell. The transcriptional unit in some embodiment, includes a gene of interest, in illustrative embodiments functionally linked to the promoter. In some embodiments, the GOI comprises an engineered signaling polypeptide, a hyaluronidase polypeptide, a checkpoint-inhibiting ligand, a nucleic acid sequence or polypeptide that stimulates an immune response, a nucleic acid sequence or polypeptide that stimulates apoptosis, a polypeptide that functions as a transporter, a polypeptide that stimulates growth, a toxic protein, a nucleic acid sequence or polypeptide that stimulates autophagy and shutdown of protein synthesis, a nucleic acid sequence or polypeptide that stimulates induction of anti-tumoral immunity, a suicide gene, a safety switch, an anti-idiotype polypeptide, any polypeptide known to be deleted or defective in diseases, CRISPR-Cas, guide RNAs, and/or an inhibitory RNA. In some embodiments, the gene of interest can encode an engineered signaling polypeptide (e.g., a CAR, TCR and/or lymphoproliferative element as disclosed elsewhere herein), a checkpoint-inhibiting ligand, a nucleic acid sequence or polypeptide that stimulates an immune response towards the modified cell, a nucleic acid sequence or polypeptide that stimulates apoptosis in the modified cell, a nucleic acid sequence or polypeptide that functions as a transporter, or is a member of a transporter construct, a nucleic acid sequence or polypeptide that stimulates growth (e.g., VEGF, GM-CSF) in the modified cell, a toxic protein, a nucleic acid sequence or polypeptide that stimulates autophagy and shutdown of protein synthesis, a nucleic acid sequence or polypeptide that stimulates induction of anti-tumoral immunity, a suicide gene (e.g., cytocidal dominant negative cyclin-G1 construct and herpes simplex virus thymidine kinase (HSV-TK)), a safety switch (e.g., recognition and/or elimination domain), an anti-idiotype polypeptide, any polypeptide known to be deleted or defective in diseases, CRISPR-Cas, guide RNAs, and/or an inhibitory RNA, for example, inhibitory RNA to a GO

In some embodiments, the GOI comprises an engineered signaling polypeptide. In some embodiments, the engineered signaling polypeptide is a chimeric antigen receptor, TCR, or lymphoproliferative element. In some embodiments, the engineered signaling polypeptide is the chimeric antigen receptor. In some embodiments, the engineered signaling polypeptide is the TCR. In some embodiments, the engineered signaling polypeptide is the lymphoproliferative element. In some embodiments, the GOI is not a targeting element or a membrane-bound hyaluronidase. In some embodiments, the GOI is a hyaluronidase (i.e. hyaluronidase polypeptide).

In any of the aspects herein that include a hyaluronidase, in some embodiments, the hyaluronidase comprises an amino acid sequence with at least 90% sequence identity to any one of SEQ ID NOs:544-1003. In any of the aspects and embodiments herein that include a hyaluronidase (i.e., hyaluronidase polypeptide), the hyaluronidase polypeptide can comprise a PH20, Hyal-1, Hyal-2, Hyal-3, Hyal-4, or Hyal-5 hyaluronidase polypeptide. In other embodiments, the hyaluronidase polypeptide comprises a PH20, Hyal-1, Hyal-2, Hyal-3, or Hyal-5 hyaluronidase polypeptide. In other embodiments, the hyaluronidase polypeptide comprises a human PH20, bovine PH20, human Hyal-1, human Hyal-2, human Hyal-3, human Hyal-4, or bovine Hyal-5 hyaluronidase polypeptide. In other embodiments, the hyaluronidase polypeptide comprises a human PH20, human Hyal-1, human Hyal-2, human Hyal-3, or bovine Hyal-5 hyaluronidase polypeptide. In other embodiments, the hyaluronidase polypeptide comprises a bovine Hyal-5 hyaluronidase polypeptide.

In any of the aspects and embodiments herein that include a hyaluronidase (i.e., hyaluronidase polypeptide), the hyaluronidase polypeptide can comprise an amino acid sequence with at least 90% sequence identity to any one of SEQ ID NOs:422-480 or SEQ ID NOs:1040-1077. In other embodiments, the hyaluronidase polypeptide comprises an amino acid sequence with at least 90% sequence identity to any one of SEQ ID NOs:422-451 or 461-480. In other embodiments, the hyaluronidase polypeptide comprises an amino acid sequence with at least 90% sequence identity to any one of SEQ ID NOs:422-451 or 461-480. In other embodiments, the hyaluronidase polypeptide comprises an amino acid sequence with at least 90% sequence identity to any one of SEQ ID NOs:422-423, 434, 443, 452, 470-471, 475-477, or 478-480. In other embodiments, the hyaluronidase polypeptide is a chimeric hyaluronidase polypeptide. In other embodiments, the hyaluronidase polypeptide comprises a first stretch of at least 20 amino acids with at least 90% sequence identity to a first hyaluronidase and a second stretch of at least 20 amino acids with at least 90% sequence identity to a second hyaluronidase. In other embodiments, the hyaluronidase polypeptide comprises from amino terminus to carboxy terminus Region 1, Region 2, Region 3, Region 4, Region 5, and Region 6, and wherein one or more Regions are from a first hyaluronidase and one or more other Regions are from a second hyaluronidase. In other embodiments, the hyaluronidase polypeptide comprises from amino terminus to carboxy terminus Region 1, Region 2, Region 3, Region 4, Region 5, and Region 6, and wherein one or more Regions are from a first hyaluronidase and one or more other Regions are from a second hyaluronidase.

In any of the aspects and embodiments herein that include a hyaluronidase (i.e., hyaluronidase polypeptide), the hyaluronidase polypeptide can comprise an amino acid sequence with at least 90% sequence identity to any one of SEQ ID NOs:544-1003. In other embodiments, the hyaluronidase polypeptide comprises an amino acid sequence with at least 90% sequence identity to any one of SEQ ID NOs:544-1003. In other embodiments, the hyaluronidase polypeptide comprises from amino terminus to carboxy terminus Region 1, Region 2, Region 3, Region 4, Region 5, and Region 6, and wherein Region 5 comprises an amino acid sequence with at least 90% sequence identity to any one of SEQ ID NOs:499, 509, 518, 528, or 538. In other embodiments, Region 1, Region 2, Region 3, Region 4, and Region 6 include amino acid sequences with at least 90% sequence identity to SEQ ID NOs:483, 485, 487, 488, and 490, respectively. In other embodiments, the hyaluronidase polypeptide comprises an amino acid sequence with at least 90% sequence identity to any one of SEQ ID NOs:554-558. In other embodiments, the hyaluronidase polypeptide comprises an amino acid sequence with at least 90% sequence identity to any one of SEQ ID NOs:555-557.

In any aspect herein that includes a virus(es) or retrovirus(es), for example a RIP(s), which in illustrative embodiments encode a hyaluronidase polypeptide or include a membrane-bound hyaluronidase polypeptide, the virus or the retrovirus, for example the RIP or each of the RIPs comprises a targeting element on its surface. In some embodiments, the targeting element binds to a cytokine, chemokine, ligand, or receptor. In some embodiments, the targeting element binds to CD3, CD4, CD8, or CCR5. In some embodiments, the targeting element is an activation element. In some embodiments, the targeting element is a binding element. In some embodiments, the targeting element is a pseudotyping element.

In one aspect provided herein is an isolated polynucleotide, comprising a nucleic acid sequence encoding a hyaluronidase polypeptide comprising an amino acid sequence with at least 90% sequence identity to any one of SEQ ID NOs:544-1003. In another aspect, provided herein is an isolated hyaluronidase polypeptide, comprising an amino acid sequence with at least 90% sequence identity to any one of SEQ ID NOs:544-1003.

In another aspect, provided herein is a recombinant viral particle, comprising comprises a viral envelope, a nucleic acid encoding a packaging signal, a Gag polypeptide (i.e. Gag), and a hyaluronidase polypeptide comprising an amino acid sequence with at least 80, 85, 90, 95, 96, 97, 98, 99, or 100% sequence identity to any one of SEQ ID Nos:544-1003.

In another aspect, provided herein is a recombinant viral particle, comprising
a) a polynucleotide comprising one or more transcriptional units, wherein each of the one or more transcriptional units is operatively linked to a promoter active in a target cell, and wherein at least one of one or more transcriptional units encodes a hyaluronidase polypeptide. In some embodiments the hyaluronidase polypeptide comprises an amino acid sequence with at least 90% sequence identity to any one of SEQ ID Nos:544-1003. In some embodiments, the hyaluronidase polypeptide is membrane-bound. Typically, the recombinant viral particle further comprises a viral envelope, a nucleic acid encoding a packaging signal, and Gag.

In some aspects, provided herein is a viral particle formulation, comprising a plurality of viral particles, wherein each of the viral particles comprises:
a) a polynucleotide comprising one or more transcriptional units, wherein each of the one or more transcriptional units is operatively linked to a promoter active in a target cell, and wherein at least one of one or more transcriptional units encodes the hyaluronidase polypeptide comprising an amino acid sequence with at least 90% sequence identity to any one of SEQ ID Nos:544-1003; and/or
b) the a hyaluronidase polypeptide comprising an amino acid sequence with at least 90% sequence identity to any one of SEQ ID NOs:544-1003, wherein the hyaluronidase polypeptide is membrane-bound.

In some aspects, provided herein is a target cell, comprising:
a) a polynucleotide comprising one or more transcriptional units, wherein each of the one or more transcriptional units is operatively linked to a promoter active in a target cell, and wherein at least one of one or more transcriptional units encodes the hyaluronidase polypeptide of claim B1; and/or
b) a hyaluronidase polypeptide comprising an amino acid sequence with at least 90% sequence identity to any one of SEQ ID NOs:544-1003 of claim B1.

In some aspects, provided herein is a replication incompetent recombinant retroviral particle (RIP), comprising:
a) a polynucleotide comprising one or more transcriptional units, wherein each of the one or more transcriptional units is operatively linked to a promoter active in a target cell, and wherein the polynucleotide comprises a nucleic acid containing a packaging motif;
b) a membrane-bound hyaluronidase polypeptide; and
c) a Gag polypeptide.

In some aspects, provided herein is a replication incompetent recombinant retroviral particle (RIP), comprising a polynucleotide comprising one or more transcriptional units, wherein each of the one or more transcriptional units is operatively linked to a promoter active in a target cell, and the RIP comprises a membrane-bound hyaluronidase.

In one aspect, provided herein is a replication incompetent recombinant retroviral particle (RIP) formulation, comprising a plurality of RIPs, wherein each of the RIPs comprises a polynucleotide comprising one or more transcriptional units, wherein each of the one or more transcriptional units is operatively linked to a promoter active in a target cell, and each of the RIPs comprises a membrane-bound hyaluronidase polypeptide.

In some embodiments, provided herein is a delivery solution, delivery reagent, RIP formulation, or pharmaceutical composition, comprising a vessel holding a solution comprising an isolated polynucleotide herein, a hyaluronidase polypeptide herein, a viral particle herein, a viral particle formulation herein, a target cell herein, or a RIP herein.

In one aspect, provided herein are viral particles for use in administering a viral particle formulation to a subject, wherein use of the viral particles comprises, administering the viral particle formulation to the subject, wherein the viral particle formulation comprises the viral particles, and wherein the viral particles are any of the viral particles disclosed herein, including for example, recombinant retroviral particles that encode a hyaluronidase polypeptide herein, and/or that include a membrane-bound hyaluronidase polypeptide.

In another aspect, provide herein are viral particles for use in modifying target cells in a subject, wherein use of the viral particles comprises administering to the subject, a viral particle formulation comprising the viral particles, wherein the viral particles are any of the viral particles disclosed herein, including for example, recombinant retroviral particles that encode a hyaluronidase polypeptide herein, and/or that include a membrane-bound hyaluronidase polypeptide, and wherein said administering facilitates association of the target cells with the viral particles,
wherein the target cells are present in the subject, and wherein the viral particles modify the target cells to form a population of modified target cells in the subject.

In any of the aspects and embodiments herein that include a hyaluronidase, including as non-limiting examples, aspects and embodiments that include a soluble hyaluronidase or a membrane-bound hyaluronidase such as attached to the surface of a virus, the hyaluronidase can be any chimeric hyaluronidase disclosed elsewhere herein. In such embodiments, a hyaluronidase polypeptide can include from amino terminus to carboxy terminus Region 1, Region 2, Region 3, Region 4, Region 5, and Region 6, as disclosed elsewhere herein. In some embodiments, the hyaluronidase polypeptide further includes a Region 0 N-terminal of Region 1. In some embodiments, the hyaluronidase polypeptide further includes a Region 7 C-terminal of Region 6. In some embodiments, the hyaluronidase polypeptide further comprises a heterologous membrane attachment sequence.

In one aspect provided herein is a hyaluronidase polypeptide, wherein Region 5 includes an amino acid sequence with at least 80%, 90%, 95%, 96%, 97%, 98%, or 99% sequence identity or identical to any one of SEQ ID NOs:499, 509, 518, 528, or 538. In some embodiments, Region 5 includes an amino acid sequence with at least 90% sequence identity to any one of SEQ ID NOs:499, 509, 518, 528, or 538. In some embodiments, Region 5 includes an amino acid sequence with at least 95% sequence identity to any one of SEQ ID NOs:499, 509, 518, 528, or 538. In some embodiments, Region 5 includes an amino acid sequence of any one of SEQ ID NOs:499, 509, 518, 528, or 538. In some embodiments, Region 1, Region 2, Region 3, Region 4, and Region 6 include amino acid sequences with at least 80%, 90%, 95%, 96%, 97%, 98%, or 99% sequence identity or identical to SEQ ID NOs:483, 485, 487, 488, and 490, respectively. In some embodiments, Region 1, Region 2, Region 3, Region 4, and Region 6 include amino acid sequences with at least 90% sequence identity to SEQ ID NOs:483, 485, 487, 488, and 490, respectively. In some embodiments, Region 1, Region 2, Region 3, Region 4, and Region 6 include amino acid sequences with at least 95% sequence identity to SEQ ID NOs:483, 485, 487, 488, and 490, respectively. In some embodiments, Region 1, Region 2, Region 3, Region 4, and Region 6 include amino acid sequences of SEQ ID NOs:483, 485, 487, 488, and 490, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:493, 503, 512, 522, or 532, and wherein Region 5 includes an amino acid sequence with at least 80%, 90%, 95%, 96%, 97%, 98%, or 99% sequence identity or identical to any one of SEQ ID NOs:499, 509, 518, 528, or 538. In some embodiments, Region 1 includes an amino acid sequence with at least 90% sequence identity to any one of SEQ ID NOs:493, 503, 512, 522, or 532. In some embodiments, Region 1 includes an amino acid sequence with at least 95% sequence identity to any one of SEQ ID NOs:493, 503, 512, 522, or 532. In some embodiments, Region 1 includes an amino acid sequence of any one of SEQ ID NOs:493, 503, 512, 522, or 532. In some embodiments, Region 5 includes an amino acid sequence with at least 90% sequence identity to any one of SEQ ID NOs:499, 509, 518, 528, or 538. In some embodiments, Region 5 includes an amino acid sequence with at least 95% sequence identity to any one of SEQ ID NOs:499, 509, 518, 528, or 538. In some embodiments, Region 5 includes an amino acid sequence of any one of SEQ ID NOs:499, 509, 518, 528, or 538. In some embodiments, Region 2, Region 3, Region 4, and Region 6 include amino acid sequences with at least 80%, 90%, 95%, 96%, 97%, 98%, or 99% sequence identity or identical to SEQ ID NOs:485, 487, 488, and 490, respectively. In some embodiments, Region 2, Region 3, Region 4, and Region 6 include amino acid sequences with at least 90% sequence identity to SEQ ID NOs:485, 487, 488, and 490, respectively. In some embodiments, Region 2, Region 3, Region 4, and Region 6 include amino acid sequences with at least 95% sequence identity to SEQ ID NOs:485, 487, 488, and 490, respectively. In some embodiments, Region 2, Region 3, Region 4, and Region 6 include amino acid sequences of SEQ ID NOs:485, 487, 488, and 490, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:493, 503, 512, 522, or 532, wherein Region 5 includes an amino acid sequence with at least 80%, 90%, 95%, 96%, 97%, 98%, or 99% sequence identity or identical to any one of SEQ ID NOs:499, 509, 518, 528, or 538, and wherein Region 6 includes an amino acid sequence with at least 80%, 90%, 95%, 96%, 97%, 98%, or 99% sequence identity or identical to any one of SEQ ID NOs:500, 510, 519, 529, or 539. In some embodiments, Region 1 includes an amino acid sequence with at least 90% sequence identity to any one of SEQ ID NOs:493, 503, 512, 522, or 532. In some embodiments, Region 1 includes an amino acid sequence with at least 95% sequence identity to any one of SEQ ID NOs:493, 503, 512, 522, or 532. In some embodiments, Region 1 includes an amino acid sequence of any one of SEQ ID NOs:493, 503, 512, 522, or 532. In some embodiments, Region 5 includes an amino acid sequence with at least 90% sequence identity to any one of SEQ ID NOs:499, 509, 518, 528, or 538. In some embodiments, Region 5 includes an amino acid sequence with at least 95% sequence identity to any one of SEQ ID NOs:499, 509, 518, 528, or 538. In some embodiments, Region 5 includes an amino acid sequence of any one of SEQ ID NOs:499, 509, 518, 528, or 538. In some embodiments, Region 6 includes an amino acid sequence with at least 90% sequence identity to any one of SEQ ID NOs:500, 510, 519, 529, or 539. In some embodiments, Region 6 includes an amino acid sequence with at least 95% sequence identity to any one of SEQ ID NOs:500, 510, 519, 529, or 539. In some embodiments, Region 6 includes an amino acid sequence of any one of SEQ ID NOs:500, 510, 519, 529, or 539. In some embodiments, Region 2, Region 3, and Region 4 include amino acid sequences with at least 80%, 90%, 95%, 96%, 97%, 98%, or 99% sequence identity or identical to SEQ ID NOs:485, 487, and 488, respectively. In some embodiments, Region 2, Region 3, and Region 4 include amino acid sequences with at least 90% sequence identity to SEQ ID NOs:485, 487, and 488, respectively. In some embodiments, Region 2, Region 3, and Region 4 include amino acid sequences with at least 95% sequence identity to SEQ ID NOs:485, 487, and 488, respectively. In some embodiments, Region 2, Region 3, and Region 4 include amino acid sequences of SEQ ID NOs:485, 487, and 488, respectively.

In one aspect provided herein is a hyaluronidase polypeptide, wherein Region 5 includes an amino acid sequence with at least 80%, 90%, 95%, 96%, 97%, 98%, or 99% sequence identity or identical to any one of SEQ ID NOs:489, 509, 518, 528, or 538. In some embodiments, Region 5 includes an amino acid sequence with at least 90% sequence identity to any one of SEQ ID NOs:489, 509, 518, 528, or 538. In some embodiments, Region 5 includes an amino acid sequence with at least 95% sequence identity to any one of SEQ ID NOs:489, 509, 518, 528, or 538. In some embodiments, Region 5 includes an amino acid sequence of any one of SEQ ID NOs:489, 509, 518, 528, or 538. In some embodiments, Region 1, Region 2, Region 3, Region 4, and Region 6 include amino acid sequences with at least 80%, 90%, 95%, 96%, 97%, 98%, or 99% sequence identity or identical to SEQ ID NOs:493, 495, 497, 498, and 500, respectively. In some embodiments, Region 1, Region 2, Region 3, Region 4, and Region 6 include amino acid sequences with at least 90% sequence identity to SEQ ID NOs:493, 495, 497, 498, and 500, respectively. In some embodiments, Region 1, Region 2, Region 3, Region 4, and Region 6 include amino acid sequences with at least 95% sequence identity to SEQ ID NOs:493, 495, 497, 498, and 500, respectively. In some embodiments, Region 1, Region 2, Region 3, Region 4, and Region 6 include amino acid sequences of SEQ ID NOs:493, 495, 497, 498, and 500, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 503, 512, 522, or 532, and wherein Region 5 includes an amino acid sequence with at least 80%, 90%, 95%, 96%, 97%, 98%, or 99% sequence identity or identical to any one of SEQ ID NOs:489, 509, 518, 528, or 538. In some embodiments, Region 1 includes an amino acid sequence with at least 90% sequence identity to any one of SEQ ID NOs:483, 503, 512, 522, or 532. In some embodiments, Region 1 includes an amino acid sequence with at least 95% sequence identity to any one of SEQ ID NOs:483, 503, 512, 522, or 532. In some embodiments, Region 1 includes an amino acid sequence of any one of SEQ ID NOs:483, 503, 512, 522, or 532. In some embodiments, Region 5 includes an amino acid sequence with at least 90% sequence identity to any one of SEQ ID NOs:489, 509, 518, 528, or 538. In some embodiments, Region 5 includes an amino acid sequence with at least 95% sequence identity to any one of SEQ ID NOs:489, 509, 518, 528, or 538. In some embodiments, Region 5 includes an amino acid sequence of any one of SEQ ID NOs:489, 509, 518, 528, or 538. In some embodiments, Region 2, Region 3, Region 4, and Region 6 include amino acid sequences with at least 80%, 90%, 95%, 96%, 97%, 98%, or 99% sequence identity or identical to SEQ ID NOs:495, 497, 498, and 500, respectively. In some embodiments, Region 2, Region 3, Region 4, and Region 6 include amino acid sequences with at least 90% sequence identity to SEQ ID NOs:495, 497, 498, and 500, respectively. In some embodiments, Region 2, Region 3, Region 4, and Region 6 include amino acid sequences with at least 95% sequence identity to SEQ ID NOs:495, 497, 498, and 500, respectively. In some embodiments, Region 2, Region 3, Region 4, and Region 6 include amino acid sequences of SEQ ID NOs:495, 497, 498, and 500, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 503, 512, 522, or 532, wherein Region 5 includes an amino acid sequence with at least 80%, 90%, 95%, 96%, 97%, 98%, or 99% sequence identity or identical to any one of SEQ ID NOs:489, 509, 518, 528, or 538, and wherein Region 6 includes an amino acid sequence with at least 80%, 90%, 95%, 96%, 97%, 98%, or 99% sequence identity or identical to any one of SEQ ID NOs:490, 510, 519, 529, or 539. In some embodiments, Region 1 includes an amino acid sequence with at least 90% sequence identity to any one of SEQ ID NOs:483, 503, 512, 522, or 532. In some embodiments, Region 1 includes an amino acid sequence with at least 95% sequence identity to any one of SEQ ID NOs:483, 503, 512, 522, or 532. In some embodiments, Region 1 includes an amino acid sequence of any one of SEQ ID NOs:483, 503, 512, 522, or 532. In some embodiments, Region 5 includes an amino acid sequence with at least 90% sequence identity to any one of SEQ ID NOs:489, 509, 518, 528, or 538. In some embodiments, Region 5 includes an amino acid sequence with at least 95% sequence identity to any one of SEQ ID NOs:489, 509, 518, 528, or 538. In some embodiments, Region 5 includes an amino acid sequence of any one of SEQ ID NOs:489, 509, 518, 528, or 538. In some embodiments, Region 6 includes an amino acid sequence with at least 90% sequence identity to any one of SEQ ID NOs:490, 510, 519, 529, or 539. In some embodiments, Region 6 includes an amino acid sequence with at least 95% sequence identity to any one of SEQ ID NOs:490, 510, 519, 529, or 539. In some embodiments, Region 6 includes an amino acid sequence of any one of SEQ ID NOs:490, 510, 519, 529, or 539. In some embodiments, Region 2, Region 3, and Region 4 include amino acid sequences with at least 80%, 90%, 95%, 96%, 97%, 98%, or 99% sequence identity or identical to SEQ ID NOs:495, 497, and 498, respectively. In some embodiments, Region 2, Region 3, and Region 4 include amino acid sequences with at least 90% sequence identity to SEQ ID NOs:495, 497, and 498, respectively. In some embodiments, Region 2, Region 3, and Region 4 include amino acid sequences with at least 95% sequence identity to SEQ ID NOs:495, 497, and 498, respectively. In some embodiments, Region 2, Region 3, and Region 4 include amino acid sequences of SEQ ID NOs:495, 497, and 498, respectively.

In one aspect provided herein is a hyaluronidase polypeptide, wherein Region 5 includes an amino acid sequence with at least 80%, 90%, 95%, 96%, 97%, 98%, or 99% sequence identity or identical to any one of SEQ ID NOs:489, 499, 509, 518, or 528. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528. In some embodiments, Region 1, Region 2, Region 3, Region 4, and Region 6 include amino acid sequences with at least 80%, 90%, 95%, 96%, 97%, 98%, or 99% sequence identity or identical to SEQ ID NOs:532, 534, 536, 537, and 539, respectively. In some embodiments, Region 1, Region 2, Region 3, Region 4, and Region 6 include amino acid sequences with at least 90% sequence identity to SEQ ID NOs:532, 534, 536, 537, and 539, respectively. In some embodiments, Region 1, Region 2, Region 3, Region 4, and Region 6 include amino acid sequences with at least 95% sequence identity to SEQ ID NOs:532, 534, 536, 537, and 539, respectively. In some embodiments, Region 1, Region 2, Region 3, Region 4, and Region 6 include amino acid sequences of SEQ ID NOs:532, 534, 536, 537, and 539, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 493, 503, 512, or 522, and wherein Region 5 includes an amino acid sequence with at least 80%, 90%, 95%, 96%, 97%, 98%, or 99% sequence identity or identical to any one of SEQ ID NOs:489, 499, 509, 518, or 528. In some embodiments, Region 1 includes an amino acid sequence with at least 90% sequence identity to any one of SEQ ID NOs:483, 493, 503, 512, or 522. In some embodiments, Region 1 includes an amino acid sequence with at least 95% sequence identity to any one of SEQ ID NOs:483, 493, 503, 512, or 522. In some embodiments, Region 1 includes an amino acid sequence of any one of SEQ ID NOs:483, 493, 503, 512, or 522. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528. In some embodiments, Region 2, Region 3, Region 4, and Region 6 include amino acid sequences with at least 80%, 90%, 95%, 96%, 97%, 98%, or 99% sequence identity or identical to SEQ ID NOs:534, 536, 537, and 539, respectively. In some embodiments, Region 2, Region 3, Region 4, and Region 6 include amino acid sequences with at least 90% sequence identity to SEQ ID NOs:534, 536, 537, and 539, respectively. In some embodiments, Region 2, Region 3, Region 4, and Region 6 include amino acid sequences with at least 95% sequence identity to SEQ ID NOs:534, 536, 537, and 539, respectively. In some embodiments, Region 2, Region 3, Region 4, and Region 6 include amino acid sequences of SEQ ID NOs:534, 536, 537, and 539, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 493, 503, 512, or 522, wherein Region 5 includes an amino acid sequence with at least 80%, 90%, 95%, 96%, 97%, 98%, or 99% sequence identity or identical to any one of SEQ ID NOs:489, 499, 509, 518, or 528, and wherein Region 6 includes an amino acid sequence with at least 80%, 90%, 95%, 96%, 97%, 98%, or 99% sequence identity or identical to any one of SEQ ID NOs:490, 500, 510, 519, or 529. In some embodiments, Region 1 includes an amino acid sequence with at least 90% sequence identity to any one of SEQ ID NOs:483, 493, 503, 512, or 522. In some embodiments, Region 1 includes an amino acid sequence with at least 95% sequence identity to any one of SEQ ID NOs:483, 493, 503, 512, or 522. In some embodiments, Region 1 includes an amino acid sequence of any one of SEQ ID NOs:483, 493, 503, 512, or 522. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528. In some embodiments, Region 6 includes an amino acid sequence with at least 90% sequence identity to any one of SEQ ID NOs:490, 500, 510, 519, or 529. In some embodiments, Region 6 includes an amino acid sequence with at least 95% sequence identity to any one of SEQ ID NOs:490, 500, 510, 519, or 529. In some embodiments, Region 6 includes an amino acid sequence of any one of SEQ ID NOs:490, 500, 510, 519, or 529. In some embodiments, Region 2, Region 3, and Region 4 include amino acid sequences with at least 80%, 90%, 95%, 96%, 97%, 98%, or 99% sequence identity or identical to SEQ ID NOs:534, 536, and 537, respectively. In some embodiments, Region 2, Region 3, and Region 4 include amino acid sequences with at least 90% sequence identity to SEQ ID NOs:534, 536, and 537, respectively. In some embodiments, Region 2, Region 3, and Region 4 include amino acid sequences with at least 95% sequence identity to SEQ ID NOs:534, 536, and 537, respectively. In some embodiments, Region 2, Region 3, and Region 4 include amino acid sequences of SEQ ID NOs:534, 536, and 537, respectively.

In one aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 493, 503, 512, or 522. In some embodiments, Region 1 includes an amino acid sequence with at least 90% sequence identity to any one of SEQ ID NOs:483, 493, 503, 512, or 522. In some embodiments, Region 1 includes an amino acid sequence with at least 95% sequence identity to any one of SEQ ID NOs:483, 493, 503, 512, or 522. In some embodiments, Region 1 includes an amino acid sequence of any one of SEQ ID NOs:483, 493, 503, 512, or 522. In some embodiments, Region 2, Region 3, Region 4, Region 5, and Region 6 include amino acid sequences with at least 80%, 90%, 95%, 96%, 97%, 98%, or 99% sequence identity or identical to SEQ ID NOs:541, 543, 537, 538, and 539, respectively. In some embodiments, Region 2, Region 3, Region 4, Region 5, and Region 6 include amino acid sequences with at least 90% sequence identity SEQ ID NOs:541, 543, 537, 538, and 539, respectively. In some embodiments, Region 2, Region 3, Region 4, Region 5, and Region 6 include amino acid sequences with at least 95% sequence identity SEQ ID NOs:541, 543, 537, 538, and 539, respectively. In some embodiments, Region 2, Region 3, Region 4, Region 5, and Region 6 include amino acid sequences of SEQ ID NOs:541, 543, 537, 538, and 539, respectively.

In another aspect provided herein is a hyaluronidase polypeptide, wherein Region 5 includes an amino acid sequence with at least 80%, 90%, 95%, 96%, 97%, 98%, or 99% sequence identity or identical to any one of SEQ ID NOs:489, 499, 509, 518, or 528. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528. In some embodiments, Region 1, Region 2, Region 3, Region 4, and Region 6 include amino acid sequences with at least 80%, 90%, 95%, 96%, 97%, 98%, or 99% sequence identity or identical to SEQ ID NOs:532, 541, 542, 537, and 539, respectively. In some embodiments, Region 1, Region 2, Region 3, Region 4, and Region 6 include amino acid sequences with at least 90% sequence identity SEQ ID NOs:532, 541, 542, 537, and 539, respectively. In some embodiments, Region 1, Region 2, Region 3, Region 4, and Region 6 include amino acid sequences with at least 95% sequence identity SEQ ID NOs:532, 541, 542, 537, and 539, respectively. In some embodiments, Region 1, Region 2, Region 3, Region 4, and Region 6 include amino acid sequences of SEQ ID NOs:532, 541, 542, 537, and 539, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 493, 503, 512, or 522, and wherein Region 5 includes an amino acid sequence with at least 80%, 90%, 95%, 96%, 97%, 98%, or 99% sequence identity or identical to any one of SEQ ID NOs:489, 499, 509, 518, or 528. In some embodiments, Region 1 includes an amino acid sequence with at least 90% sequence identity to any one of SEQ ID NOs:483, 493, 503, 512, or 522. In some embodiments, Region 1 includes an amino acid sequence with at least 95% sequence identity to any one of SEQ ID NOs:483, 493, 503, 512, or 522. In some embodiments, Region 1 includes an amino acid sequence of any one of SEQ ID NOs:483, 493, 503, 512, or 522. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528. In some embodiments, Region 2, Region 3, Region 4, and Region 6 include amino acid sequences with at least 90% sequence identity SEQ ID NOs:541, 542, 537, and 539, respectively. In some embodiments, Region 2, Region 3, Region 4, and Region 6 include amino acid sequences with at least 95% sequence identity SEQ ID NOs:541, 542, 537, and 539, respectively. In some embodiments, Region 2, Region 3, Region 4, and Region 6 include amino acid sequences of SEQ ID NOs:541, 542, 537, and 539, respectively.

In another aspect provided herein is a hyaluronidase polypeptide, wherein Region 1 includes an amino acid sequence with at least 80%, 90%, 95%, 96%, 97%, 98%, or 99% sequence identity or identical to any one of SEQ ID NOs:483, 493, 503, 512, or 522, wherein Region 5 includes an amino acid sequence with at least 80%, 90%, 95%, 96%, 97%, 98%, or 99% sequence identity or identical to any one of SEQ ID NOs:489, 499, 509, 518, or 528, and wherein Region 6 includes an amino acid sequence with at least 80%, 90%, 95%, 96%, 97%, 98%, or 99% sequence identity or identical to any one of SEQ ID NOs:490, 500, 510, 519, or 529. In some embodiments, Region 1 includes an amino acid sequence with at least 90% sequence identity to any one of SEQ ID NOs:483, 493, 503, 512, or 522. In some embodiments, Region 1 includes an amino acid sequence with at least 95% sequence identity to any one of SEQ ID NOs:483, 493, 503, 512, or 522. In some embodiments, Region 1 includes an amino acid sequence of any one of SEQ ID NOs:483, 493, 503, 512, or 522. In some embodiments, Region 5 includes an amino acid sequence with at least 90% sequence identity to any one of SEQ ID NOs:489, 499, 509, 518, or 528. In some embodiments, Region 5 includes an amino acid sequence with at least 95% sequence identity to any one of SEQ ID NOs:489, 499, 509, 518, or 528. In some embodiments, Region 5 includes an amino acid sequence of any one of SEQ ID NOs:489, 499, 509, 518, or 528.

In some embodiments, Region 6 includes an amino acid sequence with at least 90% sequence identity to any one of SEQ ID NOs:490, 500, 510, 519, or 529. In some embodiments, Region 6 includes an amino acid sequence with at least 95% sequence identity to any one of SEQ ID NOs:490, 500, 510, 519, or 529. In some embodiments, Region 6 includes an amino acid sequence of any one of SEQ ID NOs:490, 500, 510, 519, or 529. In some embodiments, Region 2, Region 3, and Region 4 include amino acid sequences with at least 90% sequence identity SEQ ID NOs:541, 542, and 537, respectively. In some embodiments, Region 2, Region 3, and Region 4 include amino acid sequences with at least 95% sequence identity SEQ ID NOs:541, 542, and 537, respectively. In some embodiments, Region 2, Region 3, and Region 4 include amino acid sequences of SEQ ID NOs:541, 542, and 537, respectively.

In any of the aspects and embodiments herein that include a hyaluronidase polypeptide including from amino to carboxy terminus Region 1, Region 2, Region 3, Region 4, Region 5, and Region 6 including, for example, a chimeric hyaluronidase polypeptide, hyaluronidase polypeptide can further include Region 0 N-terminal of Region 1. In some embodiments, Region 0 can include an amino acid sequence with at least 80%, 90%, 95%, 96%, 97%, 98%, or 99% sequence identity or identical to any one of SEQ ID NOs:482, 492, 502, 511, 521, or 531. In some embodiments, Region 0 can include an amino acid sequence with at least 90% sequence identity any one of SEQ ID NOs:482, 492, 502, 511, 521, or 531. In some embodiments, Region 0 can include an amino acid sequence with at least 95% sequence identity any one of SEQ ID NOs:482, 492, 502, 511, 521, or 531. In some embodiments, Region 0 can include an amino acid sequence of any one of SEQ ID NOs:482, 492, 502, 511, 521, or 531.

In any of the aspects and embodiments herein that include a hyaluronidase polypeptide including from amino to carboxy terminus Region 1, Region 2, Region 3, Region 4, Region 5, and Region 6 including, for example, a chimeric hyaluronidase polypeptide, hyaluronidase polypeptide can further include Region 7 C-terminal of Region 6. In some embodiments, Region 7 can include an amino acid sequence with at least 80%, 90%, 95%, 96%, 97%, 98%, or 99% sequence identity or identical to any one of SEQ ID NOs:491, 501, 520, 530, or 540. In some embodiments, Region 7 can include an amino acid sequence with at least 90% sequence identity to any one of SEQ ID NOs:491, 501, 520, 530, or 540. In some embodiments, Region 7 can include an amino acid sequence with at least 95% sequence identity to any one of SEQ ID NOs:491, 501, 520, 530, or 540. In some embodiments, Region 7 can include an amino acid sequence of any one of SEQ ID NOs:491, 501, 520, 530, or 540. In some embodiments, a hyaluronidase polypeptide including from amino to carboxy terminus Region 1, Region 2, Region 3, Region 4, Region 5, and Region 6 can further include a heterologous membrane attachment sequence C-terminal of Region 6.

In any of the aspects and embodiments herein that include RIPs, including as non-limiting examples, aspects that include delivery of a RIP formulation to a subject, the RIPs can include an activation element on the surface of the RIPs, including any of the activation elements disclosed elsewhere herein. In some embodiments, a activation element can include one or more of an antibody or an antibody mimetic capable of binding CD3, TCRα/β, CD28, or a mitogenic tetraspanin, or an activation element can be a mitogenic tetraspanin. In some embodiments, an activation element can include an antibody or an antibody mimetic capable of binding CD3. In some embodiments, activation element is an anti-CD3 antibody. In some embodiments, and wherein the activation element is bound to the membrane of the RIPs.

Provided herein in one aspect is a method for delivering, administering, and/or injecting replication incompetent recombinant retroviral particles (RIPs) to a subject, that includes the following: administering a RIP formulation comprising the RIPs to the subject, wherein the RIPs comprise:
a) an activation element associated with a membrane of the RIPs or associated with the surface or on the surface of the RIPs; and
b) a polynucleotide comprising one or more transcriptional units, wherein each of the one or more transcriptional units is operatively linked to a promoter active in T cells and/or NK cells, and wherein the one or more transcriptional units encode a lymphoproliferative element and/or a chimeric antigen receptor (CAR). In some embodiments, the one or more transcription units encode both the LE and the CAR.

Provided herein in another aspect, is a method for administering or delivering or injecting replication incompetent recombinant retroviral particles (RIPs) to a subject, said method comprising administering a RIP formulation comprising the RIPS directly to the subject, wherein the RIPs comprise:
a) an activation element associated with a membrane of the RIP; and
b) a polynucleotide comprising one or more transcriptional units, wherein each of the one or more transcriptional units is operatively linked to a promoter active in T cells and/or NK cells, and wherein the one or more transcriptional units encode a lymphoproliferative element and/or a chimeric antigen receptor (CAR), wherein the LE is constitutively active. In illustrative embodiments, the polynucleotide encodes both the LE and a chimeric antigen receptor (CAR). In some embodiments, administering a RIP formulation directly to a subject lead to the formation of modified T cells and/or NK cells in the subject, and wherein the lymphoproliferative element is expressed by the modified T cells and/or NK cells. In illustrative embodiments, expression of the lymphoproliferative element affects the phenotype (e.g., CD56+ and/or CD57+) of the modified T cells and/or NK cells in the subject. In some embodiments, at least 50%, 60%, 70%, 80%, 90%, 95%, or all of the CD4+ and CD8+ lymphocytes in the cell population express a lymphoproliferative element. In some embodiments, the lymphoproliferative element can be any of the lymphoproliferative elements disclosed elsewhere herein. In some embodiments, at least 50%, 60%, 70%, 80%, 90%, 95%, or all of the CD4+ and CD8+ lymphocytes in the cell population further express an engineered TCR and/or a CAR. In some embodiments, the engineered TCR and/or CAR can be any of the engineered TCRs and/or CARs disclosed elsewhere herein.

In any aspects and embodiments herein that include a hyaluronidase polypeptide, including, polynucleotides encoding thereof, viral particles, viral formulations, target cells, RIPs, RIPs formulations, delivery solutions, or pharmaceutical compositions, or a method thereof, the hyaluronidase polypeptide can be neutral-active. In some embodiments, the hyaluronidase polypeptide can comprise a membrane attachment sequence. In some embodiments, the membrane attachment sequence can comprise one or more of amino acid sequences SEQ ID NOs:491, 501, 520, 530, or 540. In some embodiments, the hyaluronidase polypeptide is a soluble hyaluronidase polypeptide, and in some embodiments, the hyaluronidase polypeptide comprises a signaling sequence. In some embodiments, the hyaluronidase polypeptide does not comprise a signal sequence. In some embodiments, the hyaluronidase polypeptide comprises an amino acid sequence with at least 95% sequence identity to any one of SEQ ID NOs:544-1003. In some embodiments, the hyaluronidase polypeptide comprises an amino acid sequence with at least 90% sequence identity to any one of SEQ ID NOs:422-481, or SEQ ID NOs:1040-1077, or 544-1003. In some embodiments, the hyaluronidase polypeptide comprises an amino acid sequence with at least 95% sequence identity to any one of SEQ ID NOs:422-481, or SEQ ID NOs:1040-1077 or 544-1003. In some embodiments comprising RIPs, the RIPs hyaluronidase polypeptide can comprise an amino acid sequence with at least 95% sequence identity to any one of SEQ ID NOs:422-423.

In any aspect or embodiment herein that include administering or treating a subject, the subject can have a disease. In some embodiments, the disease is selected from one or more of spinal muscular atrophy, hemophilia, Duchenne muscular dystrophy, polyneuropathy of hereditary transthyretin amyloidosis (hATTR), hyperlipoproteinaemia, amyotrophic lateral sclerosis (ALS), Huntington disease, cancer, familial partial lipodystrophy (FPL), Leber congenital amaurosis, retinal dystrophy (including biallelic retinal pigment epithelium-specific 65 kilodalton (RPE65) mutation-associated retinal dystrophy), adrenoleukodystrophy, and/or cystic fibrosis. In some embodiments, the subject has cancer, wherein the cancer can be selected from one or more of breast cancer cell, a B cell lymphoma cell, as a diffuse large B cell lymphoma (DLBCL) cell, a Hodgkin lymphoma cell, an ovarian cancer cell, a prostate cancer cell, a mesothelioma, a lung cancer cell (e.g., a small cell lung cancer cell), a lymphoma cell, a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a leukemia cell, a chronic myelogenous leukemia (CML) cell, a chronic lymphocytic leukemia (CLL) cell, an acute myelogenous leukemia (AML) cell, an acute lymphocytic leukemia (ALL) cell, a neuroblastoma cell, a glioma, a glioblastoma, a medulloblastoma, a colorectal cancer cell. In some embodiments, the cancer cell is a PDL-1 negative cell. In illustrative embodiments, the cancer cell is a PDL-1 negative DLBCL cell. In illustrative embodiments, the cancer is selected from one or more of head and neck cancer, nasopharyngeal cancer, melanoma, acute lymphocytic leukemia, mantel cell lymphoma, acute lymphocytic leukemia, metachromatic leukodystrophy, diffuse large B-cell lymphoma, follicular lymphoma, multiple myeloma, and malignant glioma.

In some embodiments of any aspect herein that include a target cell, the target cell is a T cell, a B cell, an NK cell, a dendritic cell, a macrophage, a neutrophil, a hematopoietic stem cell, an endothelial cell, a muscular vascular endothelial cell, a fibroblast, an epithelial cell, a retinal pigment epithelial cell, a motor neuron, a skeletal muscle cell, or a hepatocyte. In some embodiments, the target cell expresses one or more of CD3, CD4, CD5, CD7, CD8, CD11b, CD14, CD16, CD19, CD20, CD21, CD22, CD23, CD24, CD25, CD27, CD28, CD30, CD31, CD33, CD34, CD37, CD38, CD40, CD44, CD44v6, CD44v7/8, CD45, CD45Ra, CD5RO, CD56, CD57, CD62L, CD70, CD90, CD99, CD104, CD105, CD117, CD123, CD133, CD138, CD140, CD140b, CD146, CD171, CD184, GD2, CS-1, DC-SIGN (also known as CD209), CD11c, CD86, c-met, Eph-B2, Vap2, CXCR4, c-kit, integrins, EpCAM, CK18, recoverin, rhodopsin, cone opsin, GD2, Rlbp1, Krt8, Silv (also known as Pmel17), and/or Rrh, non-phosphorylated neurofilament-H (SMI-32), Encephalopsin (OPN3), Itm2a, and/or CD55 (DAF1), desmin, myogenin, Pax7, and/or MyoD, Asialoglycoprotein Receptor 1 (ASGR1, also known as ASGP-R 1, ASGPR 1, ASGPR1, ASGPR, C-Type Lectin Domain Family 4 Member H1 (CLEC4H1), Hepatic Lectin H1 (HL-1), CCR7, KIRs, FoxP3, and/or TCR components such as CD3A, Axl, ROR1, ROR2, Her2 (ERBB2), prostate stem cell antigen (PSCA), PSMA (prostate-specific membrane antigen), B cell maturation antigen (BCMA), alpha-fetoprotein (AFP), carcinoembryonic antigen (CEA), cancer antigen-125 (CA-125), CA19-9, calretinin, chromogranin, protein melan-A (melanoma antigen recognized by T lymphocytes; MART-1), myo-D1, muscle-specific actin (MSA), neurofilament, neuron-specific enolase (NSE), MUC-1, epithelial membrane protein (EMA), epithelial tumor antigen (ETA), tyrosinase, melanoma-associated antigen (MAGE), MAGE-A1, high molecular weight-melanoma associated antigen (HMW-MAA), placental alkaline phosphatase, synaptophysin, thyroglobulin, thyroid transcription factor-1, the dimeric form of the pyruvate kinase isoenzyme type M2 (tumor M2-PK), CD5, CD7, CD19, CD20, CD22, CD23, CD24, CD27, CD30, CD33, CD34, CD37, CD38, CD40, CD44, CD44v6, CD44v7/8, CD45, CD70, CD99, CD117, CD123, CD133, CD138, CD171, GD2 (ganglioside G2), EphA2, CSPG4, FAP (Fibroblast Activation Protein), kappa, lambda, 5T4, αvβ6 integrin, integrin αvβ3 (CD61), galactin, K-Ras (V-Ki-ras2 Kirsten rat sarcoma viral oncogene), Ral-B, B7-H3, B7-H6, CAIX, EGFR, EGP2, EGP40, EpCAM, fetal AchR, FRα, GD3, HLA-A1+MAGE1, HLA-A1+NY-ESO-1, HLA-DR, IL-11Rα, IL-13Rα2, Lewis-Y, Muc16, NCAM, NKG2D Ligands, PRAME, Survivin, TAG72, TEMs, VEGFR2, c-Met, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, an abnormal p53 protein, New York esophageal squamous cell carcinoma antigen (NYESO1), CS1, PDL-1, GPC3, GUCY2C, HER1, ICAM-1, IL13Rα2, IL11Rα, Kras, Kras G12D, LiCAM, MET, MUC1, MUC16 ecto, NKG2D, NY-ESO-1, PSCA, or WT-1 on its surface.

In some embodiments for aspects herein that include a viral particle, the viral particle is a vaccinia virus, poliovirus, adenovirus, adeno-associated virus, SV40, herpes simplex virus, rhabdovirus, reovirus, senecavirus, enterovirus, Semliki Forest virus, maraba virus, Coxsackievirus A21, Influenza A, or retrovirus. In some embodiments, the viral particle is a vaccinia virus, poliovirus, adenovirus, adeno-associated virus, or retrovirus. In some embodiments, the viral particles of any one of claims H11, H13, or VP1, wherein the viral particle is replication-incompetent. In some embodiments, the viral particles of any one of claims H11, H13, or VP1, wherein the viral particle is replication-competent.

In any aspect herein that includes a replication incompetent recombinant retroviral particle (RIP), the RIP can include a viral envelope, a nucleic acid encoding a packaging signal, Gag and a hyaluronidase polypeptide within the RIP or as a membrane-bound hyaluronidase, and/or can encode a hyaluronidase polypeptide. In some embodiments, the RIP can include POL and/or INT.

In some embodiments of any aspect that includes a viral particle or retroviral particle, including a RIP, the viral particle or retroviral particle comprises a targeting element on its surface. In some embodiments of any aspect that includes a target cell, the target cell comprises a targeting element on its surface. In some embodiments, the targeting element is any of the targeting elements provided herein. In some embodiments, the targeting element is an activation element. In some embodiments, the targeting element is a binding element. In some embodiments, the targeting element is a pseudotyping element.

In any of the aspects provided herein that include intramuscular, and in illustrative embodiments subcutaneous administration, of lymphocytes (e.g., T cells and/or NK cells), RIPs, and/or modified lymphocytes (e.g., modified T cells and/or NK cells), in certain embodiments, the subcutaneous administration is performed on a mammalian subject in a method that does not require lymphodepletion of the subject for successful engraftment in the subject and/or for successful reduction of tumor volume in the subject, or that is performed on a mammalian (e.g., human) subject that has not been subjected to lymphodepletion in the prior 1, 2, 3, 4, 5, 6, or 7 days, or prior 1, 2, 3 or 4 weeks, or prior 1, 2, 3, 6, 9, 12, or 24 months or ever before such subcutaneous administration. In certain embodiments, the subcutaneous administration is performed on a mammalian (e.g., human) subject that is not suffering from a low white blood cell count, lymphopenia or lymphocytopenia. In certain embodiments, the subcutaneous administration is performed on a subject having a lymphocyte count in the normal range (i.e., 1,000 and 4,800 lymphocytes in 1 microliter (μL) of blood). In certain embodiments, the subcutaneous administration is performed on a subject having between 1,000 and 5,000, over 300, over 500, over 1,000, over 1,500, or over 2,000 lymphocytes per μL of blood). In certain embodiments, the subcutaneous administration is performed on a mammalian (e.g., human) subject that is lymphoreplete.

In any of the aspects provided herein that include intraperitoneal, intramuscular, intranodal, and in illustrative embodiments subcutaneous administration of lymphocytes (e.g., co-administered unmodified T cells and/or unmodified NK cells), RIPs or modified lymphocytes (e.g., modified T cells and/or NK cells) any of which in illustrative embodiments comprise membrane-bound hyaluronidase and/or in formulations that include any hyaluronidase polypeptide provided herein either free in solution or bound to a membrane, certain embodiments can include a step of delivery of another component(s) such as a molecule(s) (ion(s)), macromolecule(s) (e.g., DNA, RNA, peptides, and polypeptides) and/or other cell(s), such as other modified cell(s) (e.g., genetically modified cell(s)), that can affect the modified T cells and/or NK cells, subcutaneously at or near the site of delivery of the lymphocytes (e.g., co-administered unmodified T cells and/or unmodified NK cells), RIPs or modified T cells and/or NK cells, in the same or a different formulation. In certain illustrative embodiments, the other component(s) include an antigen, a recombinant cell encoding a recombinant antigen, or an RNA encoding the antigen, or a cytokine that drives proliferation of T cells and/or NK cells. These other components, which are disclosed in more detail herein, can be delivered either in the same formulation or in different formulation(s) than the lymphocytes (e.g., co-administered unmodified T cells and/or unmodified NK cells), RIPs or modified T cells and/or NK cells. Furthermore, these other components can be delivered along with the lymphocytes (e.g., co-administered unmodified T cells and/or unmodified NK cells), RIPs or modified T cells and/or NK cells or can be delivered days (e.g., 1, 2, 3, 4, 5, 6, or 7 days), weeks (e.g., 1, 2, 4, or 4 weeks), or even months (e.g., 1, 2, 3, 6, 12, or 24 months) before or after the lymphocytes (e.g., co-administered unmodified T cells and/or unmodified NK cells), RIPs or modified T cells and/or NK cells are delivered. In some embodiments, one or more of these other components is delivered at more than one time point, such as on the same day as, or simultaneously with the lymphocytes (e.g., co-administered unmodified T cells and/or unmodified NK cells), RIPs or modified T cells and/or NK cells, and at one or more of the times recited herein above in this paragraph. Accordingly, in some embodiments a second formulation is administered to the subject at a second timepoint between 1 day and 1 month, 2 months, 3 months, 6 months, or 12 months after the administering the RIP formulation or cell formulation. The other component that is administered to the subject in addition to modified lymphocytes or substantially purified or purified RIPs, can include (e.g., i) a cytokine, for example IL-2, ii) an antibody or polypeptide that is capable of binding CD2, CD3, CD28, OX40, 4-1BB, ICOS, CD9, CD53, CD63, CD81, and/or CD82, and/or iii) a source of the cognate antigen recognized by the CAR). In certain embodiments subcutaneous administration of the lymphocytes (e.g., co-administered unmodified T cells and/or unmodified NK cells), RIPs or modified T cells and/or NK cells is performed near (e.g., within 1, 2, 3, 4, 5, 10, 20, or 30 cm) a site of neoplastic (e.g., cancerous) cells, such as a tumor, or an organ comprising a tumor, including for example, the spleen in the case of blood cancers, or where multiple administrations are performed of the same formulation, or of different formulations, they can be performed at or near the site of a prior administration or away from such site. In some embodiments, the RIP formulation or cell formulation comprises a source of a cognate antigen for the CAR, wherein the source of the cognate antigen is the cognate antigen, an mRNA encoding the cognate antigen, or a cell expressing the cognate antigen. In some embodiments, the RIP formulation or the cell formulation comprises a cytokine and wherein the cytokine is IL-2, IL-7, IL-15, or IL-21 or a modified version of any of these cytokines that is capable of binding to and activating a native receptor for the cytokine. The cognate antigen for this and any embodiments herein, including in this Exemplary Embodiments section, can be any of the tumor associated or tumor specific antigens provided herein.

In certain aspects, provided herein is a population of genetically modified T cells and/or NK cells, wherein the populations is in a subcutaneous environment in a mammal, such as a human, wherein at least 50%, 75%, 90%, 95%, 96%, 97%, 98%, or 99% of the modified T cells and/or NK cells are genetically modified, and in illustrative embodiments comprise a polynucleotide encoding a CAR integrated into their genomic DNA. In some embodiments, such a population can further comprise a gradient of the modified T cells and/or NK cells emanating from a site of intramuscular, and in illustrative embodiments subcutaneous delivery, which in some embodiments is formed days (e.g., 1, 2, 3, 4, 5, 6, or 7 days), weeks (e.g., 1, 2, 4, or 4 weeks), or months (e.g., 1, 2, 3, 6, 12, or 24 months) after the RIPs, optionally co-administered with lymphocytes (e.g., unmodified T cells and/or unmodified NK cells), or modified T cells are injected intramuscularly, or in illustrative embodiments, subcutaneously into a subject. In some embodiments another component(s) such as a molecule(s) (ion(s)), macromolecule(s) (e.g., DNA, RNA, peptides, and polypeptides) and/or other cell(s), such as other modified (e.g., genetically modified) cell(s) that can affect the modified T cells and/or NK cells that are either administered to the subject or formed after T cells and/or NK cells are modified in vivo by RIPs that were directly administered to the subject, as disclosed herein, is present in the subcutaneous environment.

The subcutaneous environment comprising modified cells, such as modified T cells and/or NK cells and/or RIPs can be, for example, 1, 2, 3, 4, 5, 6, 7, 8, 9, or 10 cm³ in volume. In some embodiments, such subcutaneous environment can be considered an in vivo reaction mixture and can include one or more hyaluronidase polypeptides in solution in the reaction mixture, and/or bound to a membrane of the modified cells and/or RIPs. In some embodiments, such subcutaneous environment comprises, for example 1×10³ to 1×10⁸ TUs/kg subject of RIPs. In some embodiments, such subcutaneous environment can comprise at least 1×10¹⁰, 1×10¹¹, 1×10¹², or 1×10¹³ genetically modified T cells and/or NK cells, or between 1×10⁹ and 1×10¹⁵ genetically modified T cells and/or NK cells, for example between 1×10¹⁰ and 1×10¹³, between 1×10¹⁰ and 1×10¹², or between 1×10¹¹ and 1×10¹³ genetically modified T cells and/or NK cells. In some embodiments, such subcutaneous environment can comprise between 1×10⁹ and 1×10¹³, for example between 1×10¹⁰ and 1×10¹³, between 1×10¹⁰ and 1×10¹², or between 1×10¹¹ and 1×10¹³ genetically modified T cells and/or modified NK cells wherein such cells comprise a polynucleotide encoding a CAR integrated into their genomic DNA. In a related aspect, provided is a mammalian subject, for example a human subject, wherein at least 50, 60, 70, 75, 80, 90, or 95% of the genetically modified T cells and/or NK cells in the subject that express a CAR, are subcutaneous, and in illustrative embodiments, are the population of genetically modified T cells and/or NK cells in a subcutaneous environment disclosed in this paragraph. In some embodiments, such mammalian subject is located outside a hospital. In some embodiments, such mammalian subject was not subjected to lymphodepletion in the prior 1, 2, 3, 4, 5, 6, or 7 days, or prior 1, 2, 3 or 4 weeks, or prior 1, 2, 3, 6, 9, 12, or 24 months or ever before.

In some aspects, provided herein are a set of viruses, retroviruses, or RIPs “for use in” aspects that are directed to viruses, retroviruses, replication incompetent recombinant retroviral particles (RIPs) that in illustrative embodiments include a membrane-bound hyaluronidase polypeptide, for use in administering, delivering or injecting a RIP formulation to a subject or for use in modifying cells, such as T cells and/or NK cells in a subject, wherein use of the RIPs comprises any of the method aspects provided herein that include administering, delivering and/or injecting RIPs to a subject, for example in RIP formulations, in modifying compositions comprising RIPs, or in delivery solutions comprising RIPs. Thus, in some aspects, each aspect of the set of viruses, retroviruses or RIPs “for use in” aspects correspond to a method aspect provided herein.

In some aspects, provided herein are a set of “use of RIPs in the manufacture of a kit” (or similarly viruses or retroviruses use language) aspects that are directed to use of replication incompetent recombinant retroviral particles (RIPs) that in illustrative embodiments include a membrane-bound hyaluronidase polypeptide, in the manufacture of a kit for modifying and/or genetically modifying T cells and/or NK cells in a subject, wherein the use of the kit comprises any of the method aspects provided herein that include administering, delivering, and/or injecting RIPs into a subject, for example in RIP formulations, in modifying compositions comprising RIPs, or in delivery solutions comprising RIPs. Thus, in some aspects each aspect of the set of “use of RIPs in the manufacture of a kit” aspects correspond to a method aspect provided herein.

It will be understood that any administering step, can in some aspects and embodiments, be an injecting step or a delivering step or introducing step. Where aspects and embodiments refer to associated with a surface, they could interchangeable refer to on a surface, associated with one or more surfaces, bound to a surface, or bound to one or more surfaces.

In one aspect, provided herein is a method for delivering a RIP formulation to a subject, wherein the method comprises administering the RIPs, RIP formulation, or pharmaceutical composition to the subject, wherein the RIP formulation comprises any of the RIPs provided herein, including in illustrative embodiments, RIPs that include a membrane-bound hyaluronidase polypeptide. In some embodiments, the RIPs comprise:
a) an activation element associated with a membrane of the RIP; and
b) a polynucleotide comprising one or more transcriptional units, wherein each of the one or more transcriptional units is operatively linked to a promoter active in target cells. In some embodiments, the target cells are T cells and/or NK cells, wherein the one or more transcriptional units encode a lymphoproliferative element and a chimeric antigen receptor (CAR), and wherein the LE is constitutively active.

In another aspect, provided herein is a method for modifying T cells and/or NK cells in a subject, wherein the method comprises, administering the RIP formulation to the subject, wherein the RIP formulation comprises the RIPs, wherein the RIPs comprise:
a) an activation element associated with a membrane of the RIP; and
b) a polynucleotide comprising one or more transcriptional units, wherein each of the one or more transcriptional units is operatively linked to a promoter active in T cells and/or NK cells, and wherein the one or more transcriptional units encode one, two, three, four or more of a lymphoproliferative element (LE) a chimeric antigen receptor (CAR), an engineered TCR, a cell removal tag, and an inhibitory RNA, wherein the RIP formulation comprises 1 ng host cell protein/TU or less and 100 TU/ng p24 protein or more. In some embodiments the transcriptional units encode an LE and CAR.

In certain embodiments, the RIP formulation is substantially free of bovine protein as disclosed in further detail herein. In other embodiments, the RIP formulation is substantially free of non-human and non-viral protein as disclosed in further detail herein.

In another aspect, provided herein is a method for modifying T cells and/or NK cells in a subject, wherein the method comprises, administering the RIP formulation to the subject, wherein the RIP formulation comprises the RIPs, wherein the RIPs comprise:
a) an activation element associated with a membrane of the RIP; and
b) a polynucleotide comprising one or more transcriptional units, wherein each of the one or more transcriptional units is operatively linked to a promoter active in T cells and/or NK cells, and wherein the one or more transcriptional units encode a lymphoproliferative element and a chimeric antigen receptor (CAR), wherein the RIP formulation comprises 0.5% to 10% colloid, 0.4 to 1.8% dextrose, 2-10% human serum albumin, and 50 to 100 mM total electrolytes, wherein the total electrolytes comprise 25 to 100 mM sodium, 0.25 to 5 mM potassium, 10 to 75 mM chloride, and 0.1 to 1.5 mM magnesium.

In some aspects, provided herein is a replication incompetent recombinant retroviral particle (RIP) formulation, comprising RIPs, wherein the RIPs comprise:
a) an activation element associated with a membrane of the RIPs; and
b) a polynucleotide comprising one or more transcriptional units, wherein each of the one or more transcriptional units is operatively linked to a promoter active in T cells and/or NK cells, wherein the one or more transcriptional units encode a lymphoproliferative element and a chimeric antigen receptor (CAR);
c) one or more membrane-bound chemokines on the surface of the RIPs, wherein the one or more membrane-bound chemokines are CCL19 and/or CCL21, or an active fragment of CCL19 and/or CCL21 capable of binding CCR7 and/or CXCR3; and
d) a membrane-bound hyaluronidase polypeptide.

In some embodiments of any of the aspects herein, for example the aspects that include administering a RIP formulation to a subject, or a RIP formulation aspect, the RIPs in the RIP formulation have a membrane-bound hyaluronidase polypeptide and the RIP formulation has a volume between 0.5 ml and 20 ml contained within a syringe. In some embodiments, the RIP formulation has a volume between 2.5 ml and 10 ml contained within a syringe. In some embodiments, the method is a method for treating a disease, and wherein the disease is cancer. In some embodiments, the administering is by perilymphatic administration. In some embodiments, the administering is by intramuscular, intratumor, intraperitoneal, intranodal, or subcutaneous administration. In some embodiments, the administering is by intranodal or subcutaneous administration. In some embodiments, between 1×10⁵ to 4×10⁹ total TUs of RIPs are present in the RIP formulation. In some embodiments, between 1×10³ to 4×10⁹ TUs/kg subject are present in the RIP formulation. In some embodiments, between 1×10⁵ to 4×10⁹ total TUs of RIPs are administered to the subject. In some embodiments, between 1×10³ to 4×10⁹ TUs/kg subject, of RIPs are administered to the subject.

Accordingly, in some aspects, provided herein is a method for delivering, administering, and/or injecting replication incompetent recombinant retroviral particles (RIPs) to a subject, said method comprises administering a RIP formulation comprising the RIPs to the subject, wherein the RIPs are any of the RIPs provided herein that include a membrane-bound hyaluronidase polypeptide. Such RIPs can further comprise a polynucleotide comprising one or more transcriptional units, wherein each of the one or more transcriptional units is operatively linked to a promoter active in T cells and/or NK cells, and wherein the one or more transcriptional units encode a lymphoproliferative element and/or a chimeric antigen receptor (CAR). In some embodiments, one or more of the transcriptional units encodes a CAR. In some embodiments the one or more transcriptional units do not encode an LE. In some embodiments, the RIPs comprise an activation element on their surface. In some illustrative embodiments, the RIPs do not comprise an activation element on their surface. In certain embodiments the method further comprises administering to the subject, a cell formulation comprising a cell suspension, wherein the cell suspension comprises suspended T cells and/or suspended NK cells, wherein the suspended T cells and/or suspended NK cells are from the subject. In some embodiments, the suspended T cells and/or suspended NK cells are in an activated state within the cell formulation. In some embodiments, the suspended T cells and/or suspended NK cells were exposed to an activation element ex vivo before being administered. In some illustrative embodiments of these embodiments wherein the suspended T cells and/or NK cells have been exposed to, and in some embodiments activated ex vivo, the RIPs do not comprise an activation element and/or the RIPs do not comprise any polynucleotides encoding an LE. In some embodiments, the suspended T cells and/or NK cells are activated ex vivo before being administered to the subject.

In some embodiments of any of the aspects herein that include delivering a RIP formulation to a subject, and some embodiments of RIP formulation aspects herein, the RIPs comprise a membrane-bound hyaluronidase polypeptide and one or more membrane-bound cytokines associated with, in illustrative embodiments bound to one or more membranes on the surface of the RIPs. Such membrane-bound cytokines can be considered targeting elements herein. In some embodiments, the membrane-bound cytokines are one or more membrane-bound chemokines. In some embodiments, the one or more membrane-bound chemokines comprise one or more of CCL1, CCL2 (MCP-1), CCL3, CCL5, CCL7 (MCP-3), CCL8 (MCP-2), CCL19, CCL20, CCL21, CCL22, CCL28, CXCL1, CXCL9, CXCL10, CXCL11, CXCL12, CXCL14 (BRAK), or CX3CL1, or a variant of any of the preceding, or an active fragment of any of the preceding. In some embodiments, the one or more membrane-bound chemokines comprise one or more polypeptides capable of binding to one or more of CCR1, CCR2, CCR4, CCR5, CCR6, CCR7, CCR8, CCR9, CXCR3, CXCR4, CXCR5, CXCR6, CCR7, or Cx3cr1. In some embodiments, at least one of the chemokines comprises a C—C motif. In some embodiments, at least one of the chemokines comprises CCL1, CCL2 (MCP-1), CCL3, CCL5, CCL7 (MCP-3), CCL8 (MCP-2), CCL19, CCL20, CCL21, CCL22, CCL28, or variants thereof, or an active fragment of any of the preceding. In some embodiments, the chemokine comprises CCL19, CCL21, or a variant thereof, or an active fragment thereof capable of binding to CCR7 or CXCR3. In some embodiments, the chemokine comprises a C-X-C motif. In some examples of such embodiments, at least one of the chemokines comprises CXCL1, CXCL9, CXCL10, CXCL11, CXCL12, CXCL14 (BRAK), or a variant of any of the preceding, or an active fragment of any of the preceding. In some embodiments, the chemokine comprises a C-X3-C motif. In some examples of such embodiments, at least one of the chemokines comprises CX3CL1, or variants thereof, or an active fragment of any of the preceding. In some examples of such embodiments, the membrane-bound chemokine comprises one or more polypeptides capable of binding to CCR1, CCR2, CCR4, CCR5, CCR6, CCR7, CCR8, CCR9, CXCR3, CXCR4, CXCR5, CXCR6, or Cx3cr1. In some examples of such embodiments, the one or more polypeptides are capable of binding to CCR7, CXCR3, CXCR4, or CXCR6. In some embodiments, the membrane-bound chemokine comprises one or more polypeptides capable of binding to CCR1, CCR2, CCR4, CCR5, CCR6, CCR7, CCR8, CCR9, CXCR3, CXCR4, CXCR5, or CXCR6. In some embodiments, the membrane-bound chemokine comprises one or more polypeptides capable of binding to CCR2, CCR5, CCR7, CCR9, CXCR3, CXCR4, CXCR6, and Cx3cr1.

In any of the method aspects provided herein that include administering a RIP formulation to a subject, or any of the RIP formulation aspects herein, the RIP formulation comprises a colloid, dextrose, albumin, and electrolytes at pH 7.2 to 7.6. In some embodiments, the RIP formulation comprises a 0.5% to 10% colloid, 0.4 to 1.8% dextrose, 2-10% albumin, and 50 to 100 mM total electrolytes. In some embodiments, the electrolytes comprise 1, 2, 3, or all of sodium, potassium, chloride, and magnesium, and wherein the albumin is human serum albumin. In some embodiments, sodium is present in the RIP formulation at between 25 to 100 mM sodium, the potassium is present in the RIP formulation at between 0.25 to 5 mM, the chloride is present in the RIP formulation at between 10 to 75 mM, and/or the magnesium is present in the RIP formulation at between 0.1 to 1.5 mM.

In any of the aspects herein that includes administering RIPs in a RIP formulation to a subject, the method further comprises, administering, delivering, or injecting a formulation comprising modified T cells and/or modified NK cells to the subject. In some embodiments, the modified T cells and/or the modified NK cells express the LE and/or the CAR.

In any of the aspects and embodiments herein that include RIPs, including as non-limiting examples, aspects that include delivery of a RIP formulation to a subject, the RIPs can include an activation element on the surface of the RIPs, including any of the activation elements disclosed elsewhere herein. In some embodiments, an activation element can include one or more of an antibody or an antibody mimetic capable of binding CD3, TCRα/β, CD28, or a mitogenic tetraspanin, or an activation element can be a mitogenic tetraspanin. In some embodiments, an activation element can include an antibody or an antibody mimetic capable of binding CD3. In some embodiments, activation element is an anti-CD3 antibody. In some embodiments, and wherein the activation element is bound to the membrane of the RIPs.

In any of the aspects and embodiments herein that include viral particles, for example, recombinant retroviral particles that encode a hyaluronidase polypeptide herein, and/or that include a membrane-bound hyaluronidase polypeptide, the RIPs can include a fusogenic element on the surface of the RIP, including any of the pseudotyping elements disclosed elsewhere herein. In some embodiments, the fusogenic element is present on a pseudotyping element. In some embodiments, a pseudotyping element further comprises a binding element. In some embodiments, a binding element of pseudotyping element is altered to reduce binding.

In any of the aspects and embodiments herein that include viral particles, for example, recombinant retroviral particles that encode a hyaluronidase polypeptide herein, and/or that include a membrane-bound hyaluronidase polypeptide, including as non-limiting examples, aspects that include delivery of a RIP formulation to a subject, the polynucleotide can encode an lymphoproliferative element (LE), including any of the LEs disclosed elsewhere herein. In some embodiments, the LE does not comprise a cytokine tethered to its cognate receptor or fragment thereof. In some embodiments, the LE does not comprise any intracellular signaling domains from IL2RA, IL2RB, IL2RG, IL7RA, IL12RB2, MyD88, OX40, GITR, or CD79B or functional mutants and/or fragments thereof. In some embodiments, the LE does not comprise more than one intracellular signaling domains from IL2RA, IL2RB, IL2RG, IL7RA, IL12RB2, MyD88, CD40, MPL, OX40, GITR, or CD79B or functional mutants and/or fragments thereof. In some embodiments, the LE does not comprise more than two intracellular signaling domains from IL2RA, IL2RB, IL2RG, IL7RA.

In some embodiments, the LE does not comprise an intracellular signaling domain from an IL-2 receptor family or functional mutants and/or fragments thereof. In some embodiments, the LE does not comprise an intracellular signaling domain from IL7RA or functional mutants and/or fragments thereof.

In some embodiments, the LE does not comprise an intracellular signaling domain from IL12RB2 or functional mutants and/or fragments thereof. In some embodiments, the LE does not comprise an intracellular signaling domain from MyD88 or functional mutants and/or fragments thereof. In some embodiments, the LE does not comprise an intracellular signaling domain from CD40 or functional mutants and/or fragments thereof. In some embodiments, the LE does not comprise an intracellular signaling domain from MPL or functional mutants and/or fragments thereof. In some embodiments, the LE does not comprise an intracellular signaling domain from OX40 or functional mutants and/or fragments thereof. In some embodiments, the LE does not comprise an intracellular signaling domain from GITR or functional mutants and/or fragments thereof. In some embodiments, the LE does not comprise an intracellular signaling domain from CD79B or functional mutants and/or fragments thereof.

In any of the aspects and embodiments herein that include viruses, for example, recombinant retroviral particles that encode a hyaluronidase polypeptide herein, and/or that include a membrane-bound hyaluronidase polypeptide, including as non-limiting examples, aspects that include delivery of a RIP formulation to a subject, the RIPs can comprise an anti-idiotype polypeptide, including any of the anti-idiotype polypeptides disclosed elsewhere herein. The anti-idiotype polypeptide can comprise an anti-idiotype extracellular recognition domain, a membrane association domain, and a stalk that connects the anti-idiotype extracellular recognition domain to the membrane association domain, wherein the anti-idiotype extracellular recognition domain comprises an idiotype-binding variable region of an anti-idiotype antibody or antibody mimetic that recognizes the idiotype of a target antibody or a target antibody mimetic. In some aspects and embodiments herein that include CARs or LEs, the CAR or LE can comprise any of the extracellular recognition domains of the anti-idiotype polypeptides disclosed herein. In some embodiments, a target antibody or target antibody mimetic of the extracellular recognition domain can be an approved biologic antibody or antibody mimetic, approved by the Food And Drug Administration of the U.S. (USFDA), European Medicines Agency (EMA), National Medical Products Administration of China (NMPA) (Chinese FDA), or the Pharmaceutical and Food Safety Bureau (PFSB) of Japan.

In any of the aspects and embodiments herein that include viruses for example, recombinant retroviral particles that encode a hyaluronidase polypeptide herein, and/or that include a membrane-bound hyaluronidase polypeptide, including as non-limiting examples, aspects that include delivery of a RIP formulation to a subject, the RIPs can further comprise on their surfaces a safety switch on their surfaces, including any of the safety switches disclosed elsewhere herein. In some embodiments, the safety switch is a recognition domain. In some embodiments, the recognition domain is recognized by a monoclonal antibody approved biologic. In some embodiments, the recognition domain comprises a polypeptide that is recognized by an antibody that recognizes EGFR, or an epitope thereof.

In any of the aspects and embodiments herein that include viruses, for example, recombinant retroviral particles that encode a hyaluronidase polypeptide herein, and/or that include a membrane-bound hyaluronidase polypeptide and comprising a polynucleotide, and/or a polynucleotide comprising one or more transcriptional units, including as non-limiting examples, aspects that include delivery of a RIP formulation to a subject, the polynucleotide and/or can transcriptional units can encode an inhibitory RNA, including any of the inhibitory RNA molecules disclosed elsewhere herein. In some embodiments, the inhibitory RNA molecules can target one or more of TCRa, TCRb, SOCS1, miR155 target, IFN gamma, cCBL, TRAIL2, PP2A, ABCG1, CD3z, PD1, CTLA4, TIM3, LAG3, SMAD2, TNFRSF10B, PPP2CA, TNFRSF6 (FAS), BTLA, TIGIT, A2AR, AHR, EOMES, SMAD3, SMAD4, TGFBR2, PPP2R2D, TNFSF6 (FASL), CASP3, SOCS2, TIEG1, JunB, Cbx3, Tet2, or HK2. In illustrative embodiments, the inhibitory RNA molecules can target one or more of AHR, Cbx3, HK2, SMAD4, or EOMES.

In some aspects, provided herein is use of replication incompetent recombinant retroviral particles including for example, recombinant retroviral particles that encode a hyaluronidase polypeptide herein, and/or that include a membrane-bound hyaluronidase polypeptide, in the manufacture of a kit for modifying and/or genetically modifying T cells and/or NK cells subcutaneously in a subject, wherein the use of the kit is a separate method aspect herein and comprises:
administering to the subject subcutaneously, a modifying composition comprising replication incompetent recombinant retroviral particles (RIPs) and an activation element, wherein the RIPs comprise a polynucleotide encoding a first polypeptide comprising a transgene, an antigen, an engineered T cell receptor, or a chimeric antigen receptor (CAR). In illustrative embodiments, the RIPs include a membrane-bound hyaluronidase polypeptide disclosed here,
wherein the modifying composition has a volume between 0.5 ml and 10 ml contained within a syringe, wherein said administering facilitates association of the T cells and/or NK cells with the RIPs, wherein the T cells and/or NK cells are present in the subcutaneous region of the subject, and wherein the RIPs modify the T cells and/or NK cells to form a population of modified T cells and/or NK cells the modifying composition.

In some embodiments, the method further comprises administering a cell suspension to the subject. Such administration can be, for example, subcutaneously. The administering the cell suspension in some embodiments, has a volume between 2 ml and 25 ml contained within a syringe, wherein the cell suspension comprises T cells and/or NK cells. In some embodiments, the RIPs in the modifying composition contact the T cells and/or NK cells, thereby modifying and/or genetically modifying the T cells and/or NK cells in the cell suspension. Such modification can occur ex vivo, for example in a syringe that includes both the RIPs and T cells and/or NK cells, or in illustrative embodiments, occurs in vivo and in some embodiments, in situ at or near the site of administration.

In some embodiments, provided herein is a method comprising administering any of the viral particles or retroviral particles, including replication incompetent recombinant retroviral particles (RIPs) provided herein, in illustrative embodiments comprising a membrane-bound hyaluronidase polypeptide, in certain embodiments along with an activation element, which in illustrative embodiments is associated with a membrane of the RIP, directly to a subject. Such administration can be, for example by intravenous, intramuscular, intratumor, intraperitoneal, intranodal, and in illustrative embodiments subcutaneous administration. The RIPs, in certain illustrative embodiments, along with an activation element can be administered in a modifying composition, wherein contacting of the RIP and lymphocytes, for example T cells and/or NK cells occurs in vivo. In some embodiments, the RIPs comprise a polynucleotide encoding a first polypeptide comprising a transgene, and in illustrative embodiments an antigen, an engineered T cell receptor, or a chimeric antigen receptor (“CAR”) wherein the CAR comprises an antibody, or fragment thereof, which in illustrative embodiments is an antigen-specific targeting region (“ASTR”), a transmembrane domain, and an intracellular activating domain, and/or a lymphoproliferative element (“LE”). In some embodiments, the RIP comprises a membrane-bound cytokine. In some embodiments, the polynucleotide encodes an anti-idiotype extracellular recognition domain. In some embodiments, the polynucleotide encodes an LE and wherein the LE is a heterodimeric LE.

In any of the compositions herein that include a hyaluronidase polypeptide, including as non-limiting examples, viral formulations the composition can further comprising a biologic and/or a small molecule therapeutic. In some illustrative embodiments, such compositions further comprising a biologic, which in some embodiments, is a virus, a retrovirus, or a cell, for example, a recombinant virus, a recombinant retrovirus, or a recombinant cell. Thus, in aspects that use such compositions, compositions disclosed herein that include a hyaluronidase polypeptide, can be co-administered with a therapeutic agent that is not associated or attached to the hyaluronidase polypeptide, such as a small molecule and/or a biologic, as non-limiting examples, therapeutic cells, antibodies, viruses, or peptides.

Additional RIP formulations and cell formulation aspects and embodiments are provided below and in the Detailed Description herein, outside this Exemplary Embodiments section. Various volumes of RIP formulations or cell formulations are provided herein for any RIP formulation or cell formulation aspect, including for example, RIP formulations wherein the RIPS therein encode a hyaluronidase polypeptide and/or include a membrane-bound hyaluronidase polypeptide. In some embodiments, the RIP formulation or cell formulation is 3 ml or greater in volume, for example 3 ml to 600 ml in volume, or between 50 ml and 500 ml, or between 100 ml and 500 ml. In some embodiments, the cell formulation or RIP formulation comprises hyaluronidase, which can be, for example, any of the hyaluronidase polypeptides disclosed herein. In some embodiments, the cell formulation or RIP formulation is 1 ml to 10 ml, 1 ml to 5 ml, 1 ml to 3 ml or 10, 5, 4, 3, or 2 ml to 25 ml, or 2 ml to 10 ml, or 2 ml to 5 ml, or 2 ml or less, or less than 3 ml, or any of the Small Volume Elements provided herein. In illustrative embodiments, the cell formulation or RIP formulation does not comprise hyaluronidase. Other volumes and formulations are provided herein. In some embodiments for any of the RIP formulation or cell formulation aspects herein, the RIP formulation or cell formulation is contained within a syringe. In some embodiments, the cell formulation, for any cell formulation provided herein, is in an incubation bag or a blood processing bag. In illustrative embodiments, the syringe is made using Good Manufacturing Practice (GMP) and is GMP grade and quality.

In some embodiments of any of the RIP formulation or cell formulation aspects provided herein, the RIP formulation or cell formulation is localized subcutaneously, intranodally, or intramuscularly, or most of the RIP formulation or cell formulation is localized subcutaneously, intranodally, or intramuscularly, in a subject. In some embodiments, the RIP formulation or cell formulation further comprises a source of the antigen recognized by the CAR. In some embodiments, the modified lymphocytes are products of a method for modifying lymphocytes provided herein.

In another aspect herein, provided herein is a method comprising administering any of the replication incompetent recombinant retroviral particles (RIPs) provided herein, typically in illustrative embodiments, encode a hyaluronidase polypeptide and/or include a membrane-bound hyaluronidase polypeptide, in certain embodiments along with an activation element, in illustrative embodiments associated with a membrane of the RIP, directly to a subject, for example by intravenous, intramuscular, intratumor, intraperitoneal, intranodal, and in illustrative embodiments subcutaneous administration. Such RIPs typically along with an activation element can be administered in a RIP formulation, which are modifying compositions comprising RIPs. In such methods, any of the contacting steps provided herein can occur in vivo. Thus, in these embodiments, the contacting typically occurs e, in some embodiments with unaltered, naturally occurring target cells (e.g., T cells and/or NK cells) present within the subject, for example that are recruited to the site of the administering. In such embodiments, RIPs can be formulated in any of the delivery solutions and any of the volumes provided herein to form the RIP formulations, which are modifying compositions comprising RIPs. Such delivery can further include administering a cell formulation, which comprise a cell suspension to the subject, wherein the cell suspension comprises T cells and/or NK cells, at or near the site on the subject of administering the RIPs, or at a different site.

Accordingly, in some embodiments, provided herein is a method, or in related aspects use of replication incompetent recombinant retroviral particles in the manufacture of a kit for modifying and/or genetically modifying cells, such as for example T cells and/or NK cells subcutaneously in a subject, wherein the method or the use of the kit comprises:
administering to the subject, in illustrative embodiments subcutaneously, a formulation comprising replication incompetent recombinant retroviral particles (RIPs) (i.e., a modifying composition comprising RIPs) that encode a hyaluronidase polypeptide and/or include a membrane-bound hyaluronidase polypeptide, and in certain embodiments an activation element, wherein the RIPs comprise a polynucleotide encoding a first polypeptide comprising a transgene, which in illustrative embodiments is a lymphoproliferative element (LE), an antigen, an engineered T cell receptor, or a chimeric antigen receptor (CAR),
wherein the modifying composition has a volume between 0.5 ml and 10 ml contained within a syringe, wherein said administering facilitates association of the T cells and/or NK cells with the RIPs, wherein the T cells and/or NK cells are present in the subcutaneous region of the subject, and wherein the RIPs modify the T cells and/or NK cells to form a population of modified T cells and/or NK cells. In illustrative embodiments, the first polypeptide is a constitutively active LE.

In some embodiments, provided herein is a method, or in related aspects use of replication incompetent recombinant retroviral particles in the manufacture of a kit for modifying and/or genetically modifying T cells and/or NK cells subcutaneously in a subject, wherein the use further comprises administering a cell suspension in a cell formulation and a RIP formulation (i.e., modifying composition comprising RIPs) to the subject subcutaneously, wherein the cell formulation and/or RIP formulation has a volume between 2 ml and 25 ml contained within a syringe, wherein the cell suspension comprises T cells and/or NK cells, wherein the RIPs in the RIP formulation (i.e., modifying composition comprising RIPs) contact the T cells and/or NK cells, thereby modifying and/or genetically modifying the T cells and/or NK cells in the cell suspension. The cell suspension can comprise T cells and/or NK cells collected earlier from the subject or allogeneic T cells and/or NK cells.

In some embodiments, the RIP formulation (i.e., modifying composition comprising RIPs) and the cell suspension in the cell formulation are administered within 0.5, 1, 2, 3, 4, or 5 cm of each other on the surface of the skin of the subject. In some embodiments, the administering the cell suspension in the cell formulation occurs simultaneously or within 1, 2, 3, 4, 5, 10, 15, 30, 45, or 60 minutes or 1, 2, 3, 4, 5, 6, 7, or 8 hours of the administering the RIP formulation (modifying composition comprising RIPs). In some embodiments, the cell suspension comprises whole blood collected from the subject. In some embodiments, the cell suspension comprises neutrophils from the subject. In some embodiments, the whole blood has been subjected to a PBMC and TNC enrichment procedure.

In some embodiments, the RIP formulation (i.e., modifying composition comprising RIPs) and the cell suspension in the cell formulation are contained within the same syringe. Thus, the solution within the syringe is both a RIP formulation and a cell formulation. In some embodiments, the activation element is a T cell activation element. In some embodiments, the T cell activation element is a polypeptide capable of binding CD3 or any of the T cell activation elements provided herein. In some embodiments, the activation element is on the surface of the RIPs. In some embodiments, the population of modified T cells and/or NK cells comprise a persisting population of genetically modified T cells and/or NK cells, wherein the persisting population of genetically modified T cells and/or NK cells persists in the subject for at least 7, 14, 21, or 28 days or 1, 2, 3, 4, 5, 6, 7, 8, 9, 10, 11, or 12 months or 1, 2, 3, 4, or 5 years after the administering the modifying composition.

In some embodiments, at least 10% of the modified T cells and/or NK cells in the population of modified T cells and/or NK cells, which in illustrative embodiments for these aspects are formed in vivo, are in cell aggregates according to any of the cell aggregate embodiments provided herein. In some embodiments, at least 50% of the CD4+ and/or CD8+ cells in the population of modified T cells and/or NK cells are CD3—according to any of the Dimmed T Cell Characteristics or Dimmed NK Cell Characteristics provided herein.

In some embodiments, the amount of a gene vector, for example a RIP, that encodes a hyaluronidase polypeptide herein, or that include a membrane-bound hyaluronidase polypeptide herein is done based on dimming units.

In any of the persisting population or population of progeny cells aspects or embodiments herein, or any aspect or embodiment herein that includes a persisting population or a population of progeny cells, the number of co-administered T cells and/or NK cells, modified cells, such as modified T cells and/or NK cells, and in illustrative embodiments genetically modified T cells and/or NK cells, comprises at least 100, 1×10³, 1×10⁴, 1×10⁵, 1×10⁶, 1×10⁷,1×10⁸, 1×10⁹, 1×10¹⁰, 1×10¹¹, or 1×10¹² cells or between 1×10³ and 1×10⁴, 1×10⁵, 1×10⁶, 1×10⁷, 1×10⁸, or 1×10⁹ cells. In some embodiments, the modified cells, and in illustrative embodiments modified T cells and/or NK cells present in modified or unmodified form in the cell formulation administered to a subject multiply at least 5, 10, 15, 20, 25, 50, 75, 100, 250, 500, 750, 1,000, 2,500, 5,000, or 10,000 fold in the subject, for example to form a persisting population or a population of progeny cells.

In some embodiments of any of the aspects provided herein, the RIP formulation or cell formulation, which in illustrative embodiments can be in a syringe, has a volume of between 0.5 ml and 20 ml, 15 ml, 10 ml, 5 ml, or 1 ml; or between 1 ml and 20 ml, 15 ml, 10 ml, 5 ml, 4 ml, 3 ml, 2 ml or less; or between 2 ml and 20 ml, 15 ml, 10 ml, 7 ml or 5 ml; or between 5 ml and 20 ml, 15 ml or 10 ml, or between 3 ml and 12 ml, or less than 3 ml

For any of the aspects provided herein that include a syringe, in illustrative embodiments, the syringe is compatible with, effective for, and/or adapted for intramuscular, and in illustrative embodiments subcutaneous delivery, and/or is effective to inject intramuscularly, effective to inject subcutaneously, adapted to inject intramuscularly, and/or adapted to inject subcutaneously. For example, the syringe can have a needle with a gauge between 20 and 22 and a length between 1 inch and 1.5 inches for intramuscular delivery and a needle with a gauge between 26 and 30 and a length between 0.5 inches and 0.625 inches for subcutaneous delivery.

In certain embodiments of any of the aspects and other embodiments herein that comprise a polynucleotide that encodes a hyaluronidase polypeptide and/or that encodes a gene of interest, which in some embodiments can be a CAR and/or an LE and include a cell, (e.g., cell formulations, populations, genetically modified lymphocytes, reaction mixtures, kits, uses of a RIP(s) in the manufacture of a kit for genetically modifying and/or transducing a lymphocyte, methods for genetically modifying and/or transducing a T cell or an NK cell, methods for administering a genetically modified lymphocyte to a subject), the cell(s) can be a tumor infiltrating lymphocyte (TIL(s). As non-limiting examples, provided herein are TILs that comprise nucleic acids encoding, individually or in combination, any CAR, recombinant TCR, LE, inhibitory RNA, cell recognition tag (e.g., anti-idiotype polypeptide), cytokine, or checkpoint inhibitor ligand disclosed herein. As further non-limiting examples, provided herein are TILs contacted with any RIP provided herein to produce a modified TIL, which in some embodiments is a genetically modified TIL.

In some embodiments, for any aspects that include a polynucleotide including one or more first transcriptional units operably linked to an inducible promoter where at least one of the one or more first transcriptional units encodes an LE, the polynucleotide can further comprise a second sequence comprising one or more second transcriptional units operably linked to a constitutive T cell or NK cell promoter, wherein at least one of the one or more second transcriptional units comprises a second polynucleotide sequence encoding a second polypeptide comprising a CAR, wherein the CAR comprises an ASTR, a transmembrane domain, and an intracellular activating domain. In some embodiments, the inducible promoter is an NFAT-responsive promoter. In some embodiments, the first and second transcriptional units are separated by the 250 cHS4 insulator (SEQ ID NO:358) in the forward orientation. In some embodiments, the RIPs are lentiviral particles.

In any of the aspects herein that include recombinant retroviral particles in a container and/or reaction mixture, the recombinant retroviral particles are present in the container and/or reaction mixture at an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15 or are present in the reaction mixture at an MOI of at least 0.1, 0.5, 1, 2, 2.5, 3, 5, 10 or 15. For kit and isolated retroviral particle embodiments, such MOI can based on 1, 2.5, 5, 10, 20, 25, 50, 100, 250, 500, or 1,000 ml assuming 1×10⁶ target cells/ml, for example in the case of whole blood, assuming 1×10⁶ PBMCs/ml of blood.

In any of the aspects herein that include a contacting cells with retroviral particles, sufficient retroviral particles are present in a reaction to achieve an MOI of between 0.1 and 50, 0.5 and 50, 0.5 and 20, 0.5 and 10, 1 and 25, 1 and 15, 1 and 10, 1 and 5, 2 and 15, 2 and 10, 2 and 7, 2 and 3, 3 and 10, 3 and 15, or 5 and 15 or at least 0.1, 0.5, 1, 2, 2.5, 3, 5, 10 or 15, or to achieve an MOI of at least 0.1, 0.5, 1, 2, 2.5, 3, 5, 10 or 15.

In any of the aspects herein that include a genetically modified T cell and/or NK cell, at least 5%, at least 10%, at least 15%, or at least 20% of the T cells and/or NK cells are genetically modified, or between 5% and 85%, or between 5% and 20%, 25%, 50%, 60%, 70%, 80%, or 85%, or between 5%, 10%, 15%, 20%, or 25% on the low end of the range, and 20%, 25%, 50%, 60%, 70%, 80%, or 85% on the high end of the range.

In any of the aspects herein that include, RIPs, the RIPs are lentiviral particles. In further illustrative embodiments, the modified cell is a modified T cell or a modified NKT cell.

In any of the aspects herein that include a polynucleotide including one or more transcriptional units, the one or more transcriptional units can encode a polypeptide comprising a CAR. In some embodiments, the CAR is a microenvironment restricted biologic (MRB)-CAR. In other embodiments, the ASTR of the CAR binds to a tumor associated antigen. In other embodiments, the ASTR of the CAR is a microenvironment-restricted biologic (MRB)-ASTR.

In certain embodiments, any of the aspects and embodiments provided herein that include a polynucleotide that comprises a nucleic acid sequences operatively linked to a promoter active in T cells and/or NK cells, the polynucleotide encodes at least one polypeptide lymphoproliferative element. In illustrative embodiments, the polypeptide lymphoproliferative element is any of the polypeptide lymphoproliferative elements disclosed herein. In some embodiments, any or all of the nucleic acid sequences provided herein can be operably linked to a riboswitch. In some embodiments, the riboswitch is capable of binding a nucleoside analog. In some embodiments, the nucleoside analog is an antiviral drug.

In any of the aspects provided herein that include a RIP, in some embodiments, the RIP comprises on its surface a nucleic acid encoding a domain recognized by a monoclonal antibody approved biologic.

In certain illustrative embodiments of any of the aspects herein that include blood cells in a reaction mixture, the blood cells in the reaction mixture are blood cells that were produced by a PBMC enrichment procedure and comprise PBMCs, or the blood cells in illustrative embodiments are PBMCs. In illustrative embodiments, such embodiments including PMBC enrichment are not combined with an embodiment where the reaction mixture includes at least 10% whole blood. Thus, in certain illustrative embodiments herein, the blood cells in a reaction mixture are the PBMC cell fraction from a PBMC enrichment procedure to which retroviral particles are added to form the reaction mixture, and in other illustrative embodiments, the blood cells in a reaction mixture are from whole blood to which retroviral particles are added to form the reaction mixture.

In any of the aspects and embodiments provided herein that include, or optionally include, a nucleic acid sequence encoding an inhibitory RNA molecule, the inhibitory RNA molecule targets any of the gene (e.g., mRNAs encoding) targets identified for example in the Inhibitory RNA Molecules section herein.

In one aspect, provided herein are methods and compositions including delivery solutions and/or formulations, for example, RIP formulations and cell formulations, for administering, delivering, introducing, or injecting RIPs or cells into a subject, wherein in illustrative embodiments the RIPs encode a hyaluronidase polypeptide and/or include membrane-bound hyaluronidase polypeptide. In another aspect, provided herein are methods for treating a disease or disorder, and in illustrative embodiments, treating cancer using delivery solutions and/or formulations provided herein. A delivery solution of the present disclosure can include RIPs, unmodified, modified, genetically modified, and/or transduced cells, RIP formulations, and/or cell formulations. In some embodiments, the RIPs of the delivery solutions and/or formulations can include any of the RIPs disclosed herein. In some embodiments, the cells of the delivery solutions and/or formulations can include any of the unmodified, modified, genetically modified, and/or transduced cells disclosed herein. In illustrative embodiments, the RIPs can include one or more polynucleotides encoding and the cells can express one or more of any of the engineered signaling polypeptides disclosed herein, e.g., any of the CARs, engineered TCRs, and/or lymphoproliferative elements disclosed elsewhere herein. In some embodiments, the RIPs and/or cells comprise on their surfaces one or more of activation elements, pseudotyping elements, binding elements, and/or fusogenic elements disclosed herein.

In some aspects and embodiments provided herein, a composition, delivery solution, and/or formulation, can comprise cells and/or RIPs and one, two, three, four, five, or more formulation components of any of the components listed herein, wherein the encode a hyaluronidase polypeptide herein, and/or that include a membrane-bound hyaluronidase polypeptide. Furthermore, the RIPs can comprise
a) an activation element associated with a membrane of the RIPs or associated with the surface or on the surface of the RIPs; and
b) a polynucleotide encoding a lymphoproliferative element (LE) and/or a chimeric antigen receptor (CAR). In some aspects and in embodiments, the delivery solution and/or formulation components can include a colloid (e.g., dextran 40, hetastarch, albumin, or PEG), sugar (e.g., dextrose, sucrose, trehalose, or lactose), HSA, saline, a buffer, and/or an electrolyte solution and in illustrative embodiments, the composition, formulation, or delivery solution includes four or more or all of these formulation components. In some aspects and in some embodiments, the delivery solution and/or formulation components can include 0.5% to 2% Dextran 40, 0.45% to 1.8% Dextrose, 22 to 100 mg/mL HSA, 1.15% to 4.6% saline, and 11% to 56% multiple electrolyte solution (e.g., Plasma-Lyte A Solution). In some embodiments, a delivery solution, RIP formulation, or cell formulation comprises 0.75% to 1.5% Dextran 40, 0.7% to 1.1% Dextrose, 30 to 60 mg/mL HSA, 2% to 2.6% saline, and 20% to 56% multiple electrolyte solution (Plasma-Lyte A Solution) at a pH of 7.0 to 7.8, 7.2 to 7.6, or 7.3 to 7.5. Percentages of components of a delivery solution and/or formulation herein can refer to either weight/volume, i.e., grams of component in 100 ml delivery solution or formulation, or volume/volume, i.e., ml component in 100 ml delivery solution or formulation. A skilled artisan can understand how to use either to make the delivery solutions and/or formulations herein.

In some embodiments, the delivery solution and/or formulation components can include a buffer, such as, PBS, HBSS, Ringer's lactate solution, or Plasma-Lyte for maintaining a target pH. In some embodiments, the delivery solution or cell formulation components further comprise unmodified, modified, genetically modified, and/or transduced cells (for example, T cells and/or NK cells). In illustrative embodiments, the delivery solution and/or cell formulation components comprise unmodified cells as disclosed herein. In some embodiments, a composition, RIP formulation, or delivery solution is co-administered with a cell formulation, for example, a cell formulation comprising unmodified cells as disclosed herein. In some embodiments, the composition, formulation, or delivery solution does not comprise DMSO. In illustrative embodiments, the composition, formulation, or delivery solution comprises a RIP containing one or more polynucleotides encoding a constitutively active LE. In further illustrative embodiments, the composition, formulation, or delivery formulation is present within a syringe.

In some embodiments, a delivery solution and/or formulation comprises 1 to 3.5%, 1.25 to 3.25%, or 1.75 to 2.5% saline. In some embodiments, a delivery solution, RIP formulation, or cell formulation comprises 1.25 to 2.5% saline. In illustrative embodiments, a delivery solution, RIP formulation, or cell formulation comprises about 2.3% saline.

In some embodiments, a delivery solution, RIP formulation, or cell formulation is or includes a multiple electrolyte solution suitable for injection into a subject. In some embodiments, a delivery solution can be or include a sterile, nonpyrogenic isotonic solution in a container, such as a single dose container. Such solution in certain embodiments is suitable or adapted for intravenous administration or intraperitoneal administration as well as subcutaneous and/or intramuscular administration. In some embodiments, a delivery solution can include a multiple electrolyte solution for injection into a subject. In illustrative embodiments, the multiple electrolyte solution comprises in each 100 mL: 526 mg of Sodium Chloride, USP (NaCl); 502 mg of Sodium Gluconate (C₆H₁₁NaO₇); 368 mg of Sodium Acetate Trihydrate, USP (C₂H₃NaO₂·3H₂O); 37 mg of Potassium Chloride, USP (KCl); and 30 mg of Magnesium Chloride, USP (MgCl₂·6H₂O) with a pH adjusted to 7.4 (6.5 to 8.0). In some embodiments, a delivery solution, RIP formulation, or cell formulation comprises 10 to 50%, 15 to 45%, 20 to 35%, 22 to 28% of the multiple analyte/electrolyte solution as described herein. In some embodiments, a RIP formulation comprises at least 10% higher multiple electrolyte solution than either of a delivery solution and cell formulation. In illustrative embodiments, a RIP formulation comprises 23 to 35%, or 23 to 29% of the multiple analyte solution. In other illustrative embodiments, a delivery solution, and a cell formulation comprises 18 to 22% of the multiple analyte solution. In illustrative embodiments, the delivery solution contains no antimicrobial agents. In some embodiments, the pH is adjusted with sodium hydroxide. In some embodiments, a multiple electrolyte injection solution can be Plasma-Lyte A Injection pH 7.4 available from various commercial suppliers. In some embodiments, the multiple electrolyte injection can be Plasma-Lyte 148. In illustrative embodiments, Plasma-Lyte can contain about 150 mM sodium, about 5 mM potassium, about 1.5 mM magnesium, about 98 mM chloride, about 27 mM acetate, and about 23 mM gluconate.

In any of the aspects and embodiments provided herein that include, or optionally include, a cell mixture, delivery solution, or formulation, the cell mixture, delivery solution, or formulation can have a pH and ionic composition. In some embodiments, the pH can be between pH 6.5 to pH 8.0, pH 7.0 to pH 8.0, or pH 7.2 to pH 7.6. In some embodiments, for example, when the RIP has a polynucleotide that encodes an MRB-CAR, the pH can be between pH 6.0 to pH 7.0, for example, pH 6.2 to pH 7.0, or pH 6.4 to pH 7.0, or pH 6.4 to pH 6.8. In some embodiments, the cell mixture, delivery solution or formulation can be maintained by a buffer such as a phosphate buffer or bicarbonate present at a concentration effective for maintaining pH in a target range. In some embodiments, a cell mixture, delivery solution or formulation can include a saline composition with salts, for example 0.8 to 1.0% or about 0.9 salts such as sodium chloride. In some embodiments, the delivery solution or formulation is or includes PBS. In some embodiments of a delivery solution or formulation herein, the concentration of Na⁺ is between 110 mM and 204 mM, the concentration of Cl⁻ is between 98 mM and 122 mM, and/or the concentration of K⁺ is between 3 mM and 6 mM. In illustrative embodiments, a delivery solution or formulation comprising the same, contains calcium and/or magnesium. The concentration of calcium can, for example, be between 0.5 mM and 2 mM. The concentration of magnesium can, for example, be between 0.5 mM and 2 mM. In some embodiments, the delivery solution or formulation is calcium and magnesium free.

In some embodiments, the total concentration of electrolytes, including, for example, sodium, potassium, chloride, and/or magnesium, in a delivery solution or formulation can be 10 to 500 mM, 25 to 250 mM, 50 to 100 mM, or 60 to 80 mM. In illustrative embodiments, the total concentration of electrolytes, including, for example, sodium, potassium, chloride, and/or magnesium can be 60 to 80 mM. In some embodiments, the total concentration of sodium in a delivery solution or formulation can be 10 to 250 mM, 25 to 100 mM, or 35 to 45 mM. In illustrative embodiments, the total concentration of sodium can be 35 to 45 mM. In some embodiments, the total concentration of potassium in a delivery solution or formulation can be 0.1 to 10 mM, 0.25 to 5, or 0.5 to 2 mM. In illustrative embodiments, the total concentration of potassium can be 0.5 to 2 mM. In some embodiments, the total concentration of chloride in a delivery solution or formulation can be 5 to 200 mM, 10 to 75 mM, or 25 to 35 mM. In illustrative embodiments, the total concentration of chloride can be 25 to 35 mM. In some embodiments, the total concentration of magnesium in a delivery solution or formulation can be 0.05 to 5 mM, 0.1 to 1.5 mM, or 0.25 to 0.75 mM. In illustrative embodiments, the total concentration of magnesium can be 0.25 to 0.75 mM.

In some embodiments, the delivery solution, RIP formulation, or cell formulation, can include Ringer's lactate solution, also known as sodium lactate solution and Hartmann's solution. In illustrative embodiments, Ringer's lactate solution can contain about 130-131 mM sodium, 109-111 mM chloride, 28-29 mM lactate, 4-5 mM potassium, and 1 to 1.5 mM calcium, and is typically made by mixing sodium chloride (NaCl), sodium lactate (CH₃CH(OH)CO₂Na), calcium chloride (CaCl₂), and potassium chloride (KCl).

In some embodiments, the delivery solution, RIP formulation, or cell formulation comprises 1% to 10%, 1% to 9%, 1% to 8%, 2% to 8%, 3% to 8%, 4% to 8%, 5% to 8%, or 5% to 7.5% DMSO. In illustrative embodiments, a delivery solution comprising RIPs or a RIP formulation does not comprise DMSO. In some embodiments, the delivery solution, RIP formulation, or cell formulation comprises 1%, 2%, 3%, 4%, 5%, 6%, 7%, 8%, 9%, or 10% or less DMSO. In certain illustrative embodiments, a delivery solution, RIP formulation, or a cell formulation comprises DMSO. In some embodiments, a delivery solution or cell formulation comprises 6% to 8% DMSO. In some embodiments, a delivery solution or cell formulation comprises 5% to 7% DMSO. In some embodiments, a delivery solution or cell formulation comprises 4% to 6% DMSO. In some embodiments, a delivery solution or a formulation can comprise a commercially available solution, such as, CryoStor 10 that comprises 10% DMSO.

In some embodiments, the delivery solution, RIP formulations, or formulation contain human serum albumin. In some embodiments the delivery solution, RIP formulation, and cell formulation contains up to 5% HSA. In some embodiments, the delivery solution, RIP formulation or cell formulation comprises 0.20% to 5%, 0.25% to 5%, 1% to 5%, 2% to 5%, or 2.5% to 5% HSA. In some embodiments, the delivery solution, RIP formulation, or cell formulation comprises 2% to 5% HSA. In some embodiments, the delivery solution, RIP formulation, or cell formulation comprises 0.2% to 2.5% HSA. In some embodiments the delivery solution is PBS comprising 2% HSA. In some embodiments, the delivery solution is DPBS comprising 2% HSA. In some embodiments, the delivery solution comprises a saline solution at about pH 7.4 further comprising HSA and sodium bicarbonate.

In some embodiments, the delivery solution, RIP formulation, or formulation comprises 30 to 100 U/ml, 40 to 100 U/ml, 30 to 60 U/ml, or 60 to 80 U/ml heparin. In some embodiments, the solution is a saline solution that further comprises heparin. In some embodiments, the delivery solution, RIP formulation, or cell formulation further comprises 0.5 to 5%, 1 to 5%, or 1 to 2.5% HSA. Discussion elsewhere herein regarding concentrations of heparin in reaction mixture aspects, apply equally to delivery solution, RIP formulation, and cell formulation aspects.

In some embodiments, the delivery solution contains at least or about 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml HSA. In some embodiments, the delivery solution, RIP formulation or cell formulation comprises 10 to 30 mg/ml, 22 to 100 mg/ml, 30 to 90 mg/ml, 35 to 75 mg/ml, 40 to 60 mg/ml, or 44 to 50 mg/ml HSA. In some embodiments, the delivery solution, RIP formulation or cell formulation comprises 40 to 60 mg/ml HSA. In some embodiments, the delivery solution, RIP formulation or cell formulation comprises 44 to 50 mg/ml HSA.

In some embodiments, a delivery solution, RIP formulation, or cell formulation comprises 0.45 to 1.8%, 0.5 to 1.5%, 0.75 to 1.25%, or 0.75 to 1% dextrose (g/100 ml). In some embodiments, a delivery solution, RIP formulation, or cell formulation comprises at least 0.5%, 1%, 2%, 3%, 4%, 5%, 6%, 7%, 8%, 9%, or 10% dextrose. In some embodiments, a delivery solution, RIP formulation, or cell formulation comprises about 0.5%, 1%, 2%, 3%, 4%, 5%, 6%, 7%, 8%, 9%, or 10% dextrose. In some embodiments, a delivery solution, RIP formulation, or cell formulation comprises 0.5% to 10%, 1% to 9%, 2% to 8%, 3% to 7%, or 4% to 6% dextrose. In some embodiments, a delivery solution, RIP formulation, or cell formulation comprises 3 to 7% dextrose. In some embodiments, a delivery solution, RIP formulation, or cell formulation comprises 3 to 6% dextrose. In illustrative embodiments, the delivery solution can be 5% dextrose in 0.9% NaCl. In other illustrative embodiments, the delivery solution can be 5% dextrose in 0.45% NaCl.

In some embodiments, a delivery solution, RIP formulation, or cell formulation comprises 0.25% to 10%, 0.5% to 10%, 0.5% to 8%, 1% to 8%, 1% to 10%, 2% to 8%, 2% to 6%, 3% to 6%, 3% to 5%, 3.5% to 4.5%, 3.6% to 4.4%, 3.7% to 4.3%, 3.8% to 4.2%, 3.9% to 4.1% or 4% lactose (g/100 ml) In some embodiments, a delivery solution, RIP formulation, or cell formulation comprises 2 to 8% lactose. In some embodiments, a delivery solution, RIP formulation, or cell formulation comprises 2 to 6% lactose.

In some embodiments, a delivery solution, RIP formulation, or cell formulation comprises 0.25% to 10%, for example 0.5% to 10%, 0.5% to 8%, 1% to 8%, 1% to 10%, 2% to 8%, 2% to 6%, 3% to 6%, 3% to 5%, 3.5% to 4.5%, 3.6% to 4.4%, 3.7% to 4.3%, 3.8% to 4.2%, 3.9% to 4.1% or4% sucrose (g/100 ml). In some embodiments, a delivery solution, RIP formulation, or cell formulation comprises 2 to 8% sucrose. In some embodiments, a delivery solution, RIP formulation, or cell formulation comprises 2 to 6% sucrose.

In some embodiments, a delivery solution, RIP formulation, or cell formulation comprises 0.25% to 10%, for example 0.5% to 10%, 0.5% to 8%, 1% to 8%, 1% to 10%, 2% to 8%, 2% to 6%, 3% to 6%, 3% to 5%, 3.5% to 4.5%, 3.6% to 4.4%, 3.7% to 4.3%, 3.8% to 4.2%, 3.9% to 4.1% or4% trehalose (g/100 ml). In some embodiments, a delivery solution, RIP formulation, or cell formulation comprises 2 to 8% trehalose. In some embodiments, a delivery solution, RIP formulation, or cell formulation comprises 2 to 6% trehalose.

In some embodiments, a delivery solution, or a formulation comprises phosphate buffered saline (PBS), and lactose. In some embodiments, the delivery solution or formulation comprises PBS and 2% to 10% lactose. In some embodiments, the delivery solution or formulation comprises PBS and 3% to 6% lactose.

In some embodiments, a delivery solution, or a formulation comprises Hanks Balanced Salt Solution (HBSS), and lactose. In some embodiments, the delivery solution or formulation comprises HBSS and 2% to 10% lactose. In some embodiments, the delivery solution or formulation comprises HBSS and 3% to 6% lactose.

In some embodiments, a delivery solution, or a formulation comprises a multiple electrolyte solution, and lactose. In some embodiments, the multiple electrolyte solution can be a commercially available solution, such as, Plasma-Lyte A. In some embodiments, the delivery solution or formulation comprises multiple electrolyte solution and 2% to 10% lactose. In some embodiments, the delivery solution or formulation comprises multiple electrolyte solution and 3% to 6% lactose.

In some embodiments, a delivery solution, or a formulation comprises phosphate buffered saline (PBS), and sucrose. In some embodiments, the delivery solution or formulation comprises PBS and 2% to 10% sucrose. In some embodiments, the delivery solution or formulation comprises PBS and 3% to 6% sucrose.

In some embodiments, a delivery solution, or a formulation comprises Hanks Balanced Salt Solution (HBSS), and sucrose. In some embodiments, the delivery solution or formulation comprises HBSS and 2% to 10% sucrose. In some embodiments, the delivery solution or formulation comprises HBSS and 3% to 6% sucrose.

In some embodiments, a delivery solution, or a formulation comprises a multiple electrolyte solution, and sucrose. In some embodiments, the multiple electrolyte solution can be a commercially available solution, such as, Plasma-Lyte A. In some embodiments, the delivery solution or formulation comprises multiple electrolyte solution and 2% to 10% sucrose. In some embodiments, the delivery solution or formulation comprises multiple electrolyte solution and 3% to 6% sucrose.

In some embodiments, a delivery solution, or a formulation comprises phosphate buffered saline (PBS), and trehalose. In some embodiments, the delivery solution or formulation comprises PBS and 2% to 10% trehalose. In some embodiments, the delivery solution or formulation comprises PBS and 3% to 6% trehalose.

In some embodiments, a delivery solution, or a formulation comprises Hanks Balanced Salt Solution (HBSS), and trehalose. In some embodiments, the delivery solution or formulation comprises HBSS and 2% to 10% trehalose. In some embodiments, the delivery solution or formulation comprises HBSS and 3% to 6% trehalose.

In some embodiments, a delivery solution, or a formulation comprises a multiple electrolyte solution, and trehalose. In some embodiments, the multiple electrolyte solution can be a commercially available solution, such as, Plasma-Lyte A. In some embodiments, the delivery solution or formulation comprises multiple electrolyte solution and 2% to 10% trehalose. In some embodiments, the delivery solution or formulation comprises multiple electrolyte solution and 3% to 6% trehalose.

In some embodiments, a delivery solution, or a formulation comprises phosphate buffered saline (PBS), and HSA. In some embodiments, the delivery solution or formulation comprises PBS and 22 to 100 mg/ml HSA. In some embodiments, the delivery solution or formulation comprises PBS and 35 to 75 mg/ml HSA.

In some embodiments, a delivery solution, or a formulation comprises Hanks Balanced Salt Solution (HBSS), and HSA. In some embodiments, the delivery solution or formulation comprises HBSS and 22 to 100 mg/ml HSA. In some embodiments, the delivery solution or formulation comprises HBSS and 35 to 75 mg/ml HSA.

In some embodiments, a delivery solution, or a formulation comprises a multiple electrolyte solution, and HSA. In some embodiments, the multiple electrolyte solution can be a commercially available solution, such as, Plasma-Lyte A. In some embodiments, the delivery solution or formulation comprises multiple electrolyte solution and 22 to 100 mg/ml HSA. In some embodiments, the delivery solution or formulation comprises multiple electrolyte solution and 35 to 75 mg/ml HSA.

In some embodiments, a delivery solution or a formulation comprises DMSO, colloid (e.g., dextran), dextrose, HSA, saline, a multiple electrolyte solution. In some embodiments, the delivery solution or formulation comprises 1 to 10% DMSO, 0.5 to 10% colloid, 0.45 to 1.8% dextrose, 20 to 100 mg/ml HSA, 1 to 3.5% saline, and 10 to 50% multiple electrolyte solution. In some embodiments, the delivery solution or formulation comprises 3 to 8% DMSO, 0.5 to 3% colloid, 0.6 to 1.4% dextrose, 35 to 65 mg/ml HSA, 1.5 to 3% saline, and 15 to 35% multiple electrolyte solution. In some embodiments, the delivery solution or formulation can further comprise a buffer as described herein.

In some embodiments, a delivery solution or a formulation comprises a colloid (e.g., dextran), dextrose, HSA, saline, a multiple electrolyte solution. In some embodiments, the delivery solution or formulation comprises 0.5 to 10% colloid, 0.45 to 1.8% dextrose, 20 to 100 mg/ml HSA, 1 to 3.5% saline, and 10 to 50% multiple electrolyte solution. In some embodiments, the delivery solution or formulation comprises 0.5 to 3% colloid, 0.6 to 1.4% dextrose, 35 to 65 mg/ml HSA, 1.5 to 3% saline, and 15 to 35% multiple electrolyte solution. In some embodiments, the delivery solution or formulation comprises 0.5 to 2% colloid, 0.7 to 1.2% dextrose, 44 to 50 mg/ml HSA, 1.75 to 2.75% saline, and 23 to 35% multiple electrolyte solution.

In some embodiments, a delivery solution or a formulation comprises a multiple analyte/electrolyte solution as described herein, dextrose, colloid (e.g., dextran 40), HAS, and DMSO. In some embodiments, the delivery solution or formulation comprises 25 to 40% (v/v) multiple analyte solution, 0.45 to 1.8% dextrose, 0.5 to 20% dextran 40, 1 to 10% HSA, and 1 to 10% DMSO. In some embodiments, the delivery solution or formulation comprises 28 to 35% (v/v) multiple analyte solution, 0.75 to 1.5% dextrose, 5 to 12% dextran 40, 3 to 7% HSA, and 4 to 9% DMSO. In some embodiments, dextrose is in a 0.45% or 0.9% sodium chloride solution. In some other embodiments, dextran is in a 5% dextrose solution.

In some embodiments, a delivery solution or a formulation comprises DMSO, sodium chloride, and HSA. In some embodiments, the delivery solution, or formulation comprises 1 to 10% DMSO, 1 to 3.5% sodium chloride, and 1 to 10% HSA. In some embodiments, the delivery solution, or formulation comprises 3 to 7% DMSO, 1 to 3% sodium chloride, and 2 to 8% HSA.

In some embodiments, a delivery solution or a formulation comprises DMSO, and a multiple electrolyte solution. In some embodiments, the delivery solution or formulation comprises 1 to 10% DMSO, and 10-70% multiple electrolyte solution. In some embodiments, the delivery solution or formulation comprises 3-7% DMSO, and 30-70% multiple electrolyte solution.

In some embodiments, a delivery solution or a formulation comprises DMSO, a multiple electrolyte solution, and HSA. In some embodiments, the delivery solution, or formulation comprises 1 to 10% DMSO, 10 to 70% multiple electrolyte solution, and 0.1-10% HSA. In some embodiments, the delivery solution, or formulation comprises 5-9% DMSO, 15 to 35% multiple electrolyte solution, and 0.1 to 1% HSA. In some embodiments, the delivery solution or formulation comprises DMSO in the form of a commercially available solution CryoStor 10, and the multiple electrolyte solution is any such solution that is commercially available for injection.

In some embodiments, a delivery solution or a formulation comprises DMSO and HSA. In some embodiments, the delivery solution or formulation comprises 1 to 10% DMSO, and 1 to 10% HSA. In some embodiments, the delivery solution or formulation comprises 3 to 7% DMSO, and 1.5 to 4% HSA.

In some embodiments, the delivery solution, RIP formulation, or cell formulation includes components that form an artificial extracellular matrix such as a hydrogel. In some embodiments, a depot delivery solution comprises an effective amount of alginate, collagen, and/or colloid, for example, dextran, to form a depot formulation. The dextran can be of a specific molecular weight within the range of, for example, 40 kDa to 2×10⁶ kDa. Other types of colloids that can be used are hetastarch, albumin, and PEG. In some embodiments, PEG can be in the range of 5 kDa to 100 kDa. In some embodiments, the delivery solution, RIP formulation, or cell formulation comprises 0.5 to 10%, 0.75 to 7.5%, or 1 to 5% colloid. In some embodiments, the polymers used to make gel-forming biomaterials, and can be included in delivery solutions and cell formulations provided herein, is composed of poly(ethylene glycol) (PEG) and its copolymers with aliphatic polyesters, such as poly(lactic acid) (PLA), poly(D,L-lactic-co-glycolic acid) (PLGA), poly(ε-caprolactone) (PCL) and polyphosphazenes. In some embodiments, the polymers used to make gel-forming biomaterials, and can be included in delivery solutions and cell formulations provided herein, include thermosensitive triblock copolymers based on poly(N-(2-hydroxypropyl methacrylamide lactate) and poly(ethylenglycol) (p(HPMAm-lac)-PEG), capable of spontaneous self-assembling in physiological environments (Vermonden et. al 2006, Langmuir 22: 10180-10184).

In some embodiments, the hydrogel used in a delivery solution, RIP formulation, or cell formulation herein, contains hyaluronic acid (HA). Such HA can have carboxylic acid groups that can be modified with 1-ethyl-3-(3-dimethyl aminopropyl)-1-carbodiimide hydrochloride to react with amine groups on proteins, peptides, polymers, and linkers, such as those found on modified lymphocytes provided herein, preferentially in the presence of N-hydroxysuccinimide. In some embodiments, antibodies, cytokines and peptides are chemically conjugated to HA using such methods to produce a hydrogel for co-injection as a cell emulsion in some RIP formulation and cell formulation embodiments provided herein. Additionally, in some embodiments, HA in delivery solutions, RIP formulations, and cell formulations is a polymer (e.g., Healon) and/or are crosslinked (e.g., restylane (AbbVie/Allergan)), for example lightly crosslinked, through its —OH groups with agents such as glutaraldehyde to reduce the local catabolism of the material following subcutaneous injection. In some embodiments, the HA used in delivery solutions, RIP formulations, and cell formulations herein, can be of variable length and viscosity. In some embodiments, the HA used in delivery solutions, RIP formulations, and cell formulations herein, can further be crosslinked with other glycosaminoglycans such as chondroitin sulfate (e.g., Viscoat) or polymers or surfactants. A skilled artisan will recognize that the porosity of the matrix and degree of crosslinking can be regulated to ensure cells, such as modified lymphocytes herein, are capable of migration through the hydrogels. Accordingly, a matrix, such as a hydrogel matrix, when used in a RIP formulation or cell formulation herein, can be configured for, or adapted to permit migration of cells through the matrix. In some embodiments, the shear modulus is or is about 2.5 kPa, about 3 kPa, about 3.5 kPa, or about 4 kPa.

In illustrative embodiments of any of the kits, delivery solutions, RIP formulations, and/or cell formulations provided herein, especially those that effective for, or adapted for intramuscular and in illustrative embodiments subcutaneous delivery, the delivery solution, RIP formulation, and/or cell formulation is a depot formulation, or the RIP formulation and/or cell formulation is an emulsion of cells that promotes cell aggregation. In some embodiments, a depot delivery solution comprises an effective amount of alginate, hydrogel, PLGA, a cross-linked and/or polymer hyaluronan, PEG, collagen, and/or dextran to form a depot formulation. In some embodiments the delivery solution, RIP formulation, and/or cell formulation is designed for controlled or delayed release. In some embodiments, the delivery solution, RIP formulation, and/or cell formulation includes components that form an artificial extracellular matrix such as a hydrogel.

In some embodiments of any of the delivery solutions and/or formulations provided herein, the delivery solution and/or formulation can be substantially free of bovine protein. For RIP formulations and/or delivery solutions comprising RIPs, substantially free of bovine protein can include having less than 50 pg bovine protein/TU. For cell formulations and/or delivery solutions comprising human cells, substantially free of bovine protein can include having less than 50 pg bovine protein/1 g human cell protein. In illustrative embodiments, the delivery solution and/or formulation is free of bovine protein, i.e., bovine protein is not detectable. In some embodiments comprising RIPs, the ratio of bovine protein to TUs can be 10, 5, 3, 2, or 1 ng or less bovine protein/TU or 750, 500, 400, 300, 200, 100, 50, 40, 30, 20, or 10 μg or less bovine protein/TU. In some embodiments comprising human cells, the ratio of bovine protein to human protein can be 10, 5, 3, 2, or 1 ng or less bovine protein/μg human protein or 750, 500, 400, 300, 200, 100, 50, 40, 30, 20, or 10 μg or less bovine protein/μg human protein.

In some embodiments of any of the delivery solutions and/or formulations provided herein, the delivery solution and/or formulation can be substantially free of non-human and non-viral protein. For RIP formulations and delivery solutions comprising RIPs, substantially free of non-human and non-viral protein can include having less than 50 pg non-human and non-viral protein/TU. For cell formulations and/or delivery solutions comprising human cells, substantially free of non-human and non-viral protein can include having less than 50 pg non-human and non-viral protein/1 jg human cell protein. In illustrative embodiments, the delivery solution and/or formulation is free of non-human and non-viral protein, i.e., non-human and non-viral protein is not detectable. In some embodiments comprising RIPs, the ratio of non-human and non-viral protein to TUs can be 10, 5, 3, 2, or 1 ng or less non-human and non-viral protein/TU or 750, 500, 400, 300, 200, 100, 50, 40, 30, 20, or 10 μg or less non-human and non-viral protein/TU. In some embodiments comprising human cells, the ratio of non-human and non-viral protein to human protein can be 10, 5, 3, 2, or 1 ng or less non-human and non-viral protein/μg human protein or 750, 500, 400, 300, 200, 100, 50, 40, 30, 20, or 10 μg or less non-human and non-viral protein/μg human protein.

Provided herein in one aspect is a cell formulation (i.e., delivery composition), comprising a delivery solution formulated with tumor infiltrating lymphocytes (TILs) and/or other modified or unmodified lymphocytes, in illustrative embodiments T cells and/or NK cells, wherein the cell formulation is compatible with, effective for, and/or adapted for subcutaneous or intramuscular delivery. In some embodiments for any of the cell formulations provided herein, the cell formulation is localized subcutaneously, or most of the cell formulation is localized subcutaneously, in a subject. In some embodiments, the cell formulation is localized subcutaneously or intramuscularly, or most of the cell formulation is localized subcutaneously or intramuscularly, in a subject. In some embodiments, wherein the cell formulation comprises TILs, the cell formulation can further comprise modified lymphocytes modified by either or both, being associated with a recombinant nucleic acid vectors, in illustrative embodiments a RIP, comprising a polynucleotide comprising one or more transcriptional units operatively linked to a promoter active in T cells and/or NK cells, or by being genetically modified with the polynucleotide, wherein the one or more transcriptional units encode a first polypeptide comprising a first CAR. In some embodiments, wherein the cell formulation comprises TILs, the cell formulation further comprises a source of a tumor antigen recognized by the TILs. In some embodiments, the TILs are contacted with a nucleic acid vector.

In some embodiments, the persisting population of genetically modified T cells and/or NK cells persists in the subject for at least 15, 21, or 28 days or 1, 2, or 1, 2, 3, 4, 5, 6, 7, 8, 9, 10, 11, or 12 months or 1, 2, 3, 4, or 5 years after administration, and wherein at least 50%, 60%, 70%, 80%, 90% or 95% of the CAR-T cells expressing the first polypeptide comprising the transgene, and in illustrative embodiments the engineered T cell receptor, or the chimeric antigen receptor (CAR) are circulating in the blood and/or at the site of a tumor, for example a solid tumor.

In some embodiments, the persisting populations persist a) subcutaneously for at least or up to 14, 21, 28, 50, 60, 90 days as detectable by histology, or b) in the blood for 3, 6, 9, or 12 months, or 1.5, 2, 2.5, 5 or 10 years as detectable by at least 2 CART/μl blood by FACs or qPCR for hRNAseP.

In some embodiments, a delivery solution, RIP formulation, or cell formulation is frozen before being thawed and administered to a subject. In some embodiments, the frozen delivery solution, RIP formulation, or cell formulation is stored at a temperature less than 0° C. for 1, 2, 3, 4, 5, 6, 7, 8, 9, 10, 11, 12, 13, or 14 days or less before being administered to a subject. In some embodiments, the frozen delivery solution, RIP formulation, or cell formulation is stored at a temperature less than −15° C. for 1, 2, 3, 4, 5, 6, 7, 8, 9, 10, 11, 12, 13, or 14 days or less before being administered to a subject. In some embodiments, the frozen delivery solution, RIP formulation, or cell formulation is stored at a temperature less than −70° C. for 1, 2, 3, 4, 5, 6, 7, 8, 9, 10, 11, 12, 13, or 14 days or less before being administered to a subject. In illustrative embodiments, the frozen delivery solution, RIP formulation, or cell formulation is stored at a temperature less than −70° C. for 12 days or less before being administered to a subject. In other illustrative embodiments, the frozen delivery solution, RIP formulation, or cell formulation is stored at a temperature less than −70° C. for 7 days or less before being administered to a subject. In further illustrative embodiments, the frozen delivery solution, RIP formulation, or cell formulation is stored at a temperature less than −70° C. for 4 days or less before being administered to a subject. In some embodiments, the frozen delivery solution, RIP formulation, or cell formulation is stored at a temperature less than −15° C. for 12 days or less before being administered to a subject. In other illustrative embodiments, the frozen delivery solution, RIP formulation, or cell formulation is stored at a temperature less than −15° C. for 7 days or less before being administered to a subject. In further illustrative embodiments, the frozen delivery solution, RIP formulation, or cell formulation is stored at a temperature less than −15° C. for 4 days or less before being administered to a subject. In some embodiments, the delivery solution and/or formulation can be frozen for 1, 2, 3, 4, 5, or 6 days, or 1, 2, 3, or 4 weeks, or 1, 2, 3, 4, 5, 6, 9, or 12 months, or indefinitely, before they are administered to, or in illustrative embodiments of cell formulations and delivery solutions comprising cells, re-administered back to the subject. During the time period in which the RIPs or cells are frozen, or any time before administration to the subject, or re-administration back to the subject, the RIPs and/or cells can be tested for various quality control attributes disclosed elsewhere herein, for example, viral concentration, purity, and/or potency, and/or one or more cell and/or gene therapy quality control tests.

In other embodiments, a delivery solution, RIP formulation, or cell formulation is never frozen before being administered to a subject. In some embodiments, the delivery solution, RIP formulation, or cell formulation is stored at 2 to 8° C. before being administered to a subject. In some embodiments, the delivery solution, RIP formulation, or cell formulation is stored at 2 to 8° C. for 1, or less before being administered to a subject. In some embodiments, the delivery solution, RIP formulation, or cell formulation is stored at 2 to 8° C. for 12 days or less before being administered to a subject. In some embodiments, the delivery solution, RIP formulation, or cell formulation is stored at 2 to 8° C. for 7 days or less before being administered to a subject. In some embodiments, the delivery solution, RIP formulation, or cell formulation is stored at 2 to 8° C. for between 1, 2, 3, 4, 5, 6, or 7 days on the low end of the range and 2, 3, 4, 5, 6, 7, 8, 9, 10, 11, 12, 13, or 14 days on the high end of the range before being administered to a subject. In some embodiments, the delivery solution, RIP formulation, or cell formulation can be stored at 2 to 8° C. for 2 to 7 days before being administered to a subject. In some embodiments, the delivery solution, RIP formulation, or cell formulation is stored at 2 to 8° C. for 2 to 7 days before being administered to a subject. In some embodiments, the delivery solution and/or formulation can be stored at 2 to 8° C. for 1, 2, 3, 4, 5, or 6 days, or 1, 2, 3, or 4 weeks, or 1, 2, 3, 4, 5, 6, 9, or 12 months, or indefinitely, before they are administered to, or in illustrative embodiments of cell formulations and delivery solutions comprising cells, re-administered back to the subject. During the time period in which the RIPs or cells are stored at 2 to 8° C., or any time before administration to the subject, or re-administration back to the subject, the RIPs and/or cells can be tested for various quality control attributes disclosed elsewhere herein, for example, viral concentration, purity, and/or potency, and/or one or more cell and/or gene therapy quality control tests.

In addition to any of the method aspects and embodiments provided herein, further provided herein are use aspects and embodiments, comprising use of a kit for performing the method, or use of nucleic acid vectors, in illustrative embodiments RIPs, in the manufacture of a kit for performing the method, wherein the use of the kit is to perform the steps of the method aspects or embodiments. For example, in one aspect, provided herein is a method for preparing a cell formulation comprising the C/F steps that comprise nucleic acid vectors, and in illustrative embodiments RIPs, in the contacting step. Such methods can optionally include the administering step above or any administering step herein. Accordingly, further provided herein is use of nucleic acid vectors, and in illustrative embodiments replication incompetent recombinant retroviral particles, in the manufacture of a kit for preparing a cell formulation, wherein use of the kit comprises performing the C/F and optional “A” steps. Similarly, for any use aspects and embodiments provided herein, further provided herein are method aspects and embodiments, comprising the method as recited in the use aspects or embodiments.

In some aspects or embodiments of a method for administrating RIPs and/or cells to a subject, the method comprises administering at least 0.1 ml, 0.2 ml, 0.3 ml, 0.5 ml, 1 ml, 1.5 ml, 2 ml, 2.5 ml, 3 ml, 4 ml, 5 ml, 6 ml, 7 ml, 8 ml, 9 ml, 10 ml, 11 ml, 12 ml, 13 ml, 14 ml, or 15 ml of any of the formulations or a delivery solutions as disclosed herein to the subject. In some embodiments, the method for administrating RIPs and/or cells to a subject, comprises administering 0.1 to 20 ml, 0.5 to 15 ml, 1 to 12 ml, 1 to 10 ml, 1 to 8 ml, or 1 to 5 ml of a formulation or a delivery solution to the subject. In some embodiments, the method for administrating RIPs and/or cells to a subject, comprises administering 0.5 to 15 ml of a formulation or a delivery solution to the subject. In some embodiments, the method for administrating RIPs and/or cells to a subject, comprises administering 0.25 to 10 ml of a formulation or a delivery solution to the subject. In some embodiments, the method for administrating RIPs and/or cells to a subject, comprises administering 0.5 to 5 ml of a formulation or a delivery solution to the subject. In some embodiments, the method for administrating RIPs and/or cells to a subject, comprises administering 2 to 3 ml of a formulation or a delivery solution to the subject. In some embodiments, a method for administering RIPs and/or cells to a subject comprises administering 0.1 ml to 20 ml of a formulation or a delivery solution to the lymph node of the subject. In illustrative embodiments, the method for administering RIPs and/or cells to a subject comprises administering 0.5 to 5 ml of a formulation or a delivery solution to the lymph node of the subject. In illustrative embodiments, the method for administering RIPs and/or cells to a subject comprises administering subcutaneously 0.5 to 5 ml of a formulation or a delivery solution to the lymph node of the subject.

In some embodiments, a method for administering RIPs to a subject comprises administering at least 10⁵, 10⁶, 10⁷, 10⁸, 10⁹, 10¹⁰, 10¹¹, 10¹², 10¹³, or 10¹⁴ total TU to the subject. In some embodiments, a method for administering RIPs to a subject comprises administering 1000 to 10¹⁴, 10⁴ to 10¹², 10⁵ to 10¹⁰, 10⁵ to 10⁹, or 10⁵ to 10⁸ total TU to the subject. In some embodiments, a method for administering RIPs to a subject comprises administering 1×10⁶ to 4×10⁹ total TU to the subject. In some embodiments, a method for administering RIPs to a subject comprises administering 1×10⁶ to 1×10⁹ total TU to the subject to the subject. In some embodiments, a method for administering RIPs to a subject comprises administering 1×10⁶ to 1×10⁸ total TU to the subject to the subject. In some embodiments, a method for administering RIPs to a subject comprises administering 5×10⁶ to 5×10⁷ total TU to the subject to the subject.

In some embodiments, a method for administering RIPs to a subject comprises administering at least 1, 10, 100, 1000, 10⁴, 10⁵, 10⁶, 10⁷, 10⁸, 10⁹, 10¹⁰, 10¹¹, 10¹², 10¹³, or 10¹⁴ transducing units (TU)/kg to the subject. In some embodiments, the method for administering RIPs to a subject comprises administering 1 to 10¹⁴ TU/kg, 10 to 10¹⁴ TU/kg, 100 to 10¹⁴ TU/kg, 1000 to 10¹⁴ TU/kg, 10⁴ to 10¹⁴ TU/kg, 10⁴ to 10¹³ TU/kg, 10⁴ to 10¹² TU/kg, 10⁵ to 10¹² TU/kg, 10⁶ to 10¹⁰ TU/kg, 10¹¹ to 10⁸ or 10⁷ to 10¹⁰ or 1×10³ to 4×10⁹ TU/kg to the subject. In some embodiments, the method for administering RIPs to a subject comprises administering 1000 to 10¹⁴ TU/kg to the subject. In some embodiments, the method for administering RIPs to a subject comprises administering 10⁴ to 10¹³ TU/kg to the subject. In some embodiments, the method for administering RIPs to a subject comprises administering 10⁶ to 10¹² TU/kg to the subject. In some embodiments, the method for administering RIPs to a subject comprises administering 10′ to 10¹¹ TU/kg to the subject.

In some embodiments, a method for administering RIPs to a subject comprises administering at least 1000, 10⁴, 10⁵, 10⁶, 10⁷, 10⁸, 10⁹, 10¹⁰, 10¹¹, 10¹², 10¹³, or 10¹⁴, transducing units (TU)/target cell/ml of blood to the subject. In some embodiments, the method for administering RIPs to a subject comprises administering 1 to 10¹⁴, 10 to 10¹⁴, 100 to 10¹⁴, 1000 to 10¹⁴, 10⁴ to 10¹⁴, 10⁴ to 10¹³, 10⁴ to 10¹², 10⁵ to 10¹², 10⁶ to 10¹⁰, or 10⁷ to 10¹⁰ TU/target cell/ml blood to the subject. In some embodiments, the method for administering RIPs to a subject comprises administering 10⁴ to 10¹³ TU/target cell/ml blood to the subject. In some embodiments, the method for administering RIPs to a subject comprises administering 10⁶ to 10¹² TU/target cell/ml blood to the subject. In some embodiments, the method for administering RIPs to a subject comprises administering 10⁷ to 10¹⁰ TU/target cell/ml blood to the subject.

In some embodiments, a method for administering RIPs to a subject comprises administering at least 1, 10, 50, 75, 100, 125, 150, 175, 200, 250, 300, 350, 400, 450, 500, 550, 600, 700, or 800 ng/kg/day to the subject. In some embodiments, a method for administering RIPs to a subject comprises administering 1 ng/kg/day to 800 mg/kg/day. In some embodiments, a method for administering RIPs to a subject comprises administering 100 ng/kg/day to 600 mg/kg/day. In some embodiments, a method for administering RIPs to a subject comprises administering 200 ng/kg/day to 500 mg/kg/day. In some embodiments, a method for administering RIPs to a subject comprises administering 300 ng/kg/day to 500 mg/kg/day.

In some embodiments, a method for administering RIPs to a subject comprises administering at least 1.0×10², 1.0×10³, 1.0×10⁴, 1.0×10⁵, 1.0×10⁶, 1.0×10⁷, 1.0×10⁸, 1.0×10⁹, 1.0×10¹⁰, or 1.0×10¹¹ GC to the subject. In some embodiments, a method for administering RIPs to a subject comprises administering 1.0×10² to 1.0×10¹⁵ GC to the subject. In some embodiments, a method for administering RIPs to a subject comprises administering 1.0×10⁴ to 1.0×10¹³ GC to the subject. In some embodiments, a method for administering RIPs to a subject comprises administering 1.0×10⁶ to 1.0×10¹² GC to the subject. In some embodiments, a method for administering RIPs to a subject comprises administering 1.0×10⁷ to 1.0×10¹⁰ GC to the subject.

In some embodiments, a method for administering RIPs to a subject comprises administering at least 1.0×10², 1.0×10³, 1.0×10⁴, 1.0×10⁵, 1.0×10⁶, 1.0×10⁷, 1.0×10⁸, 1.0×10⁹, 1.0×10¹⁰, or 1.0×10¹¹ GC/kg to the subject. In some embodiments, a method for administering RIPs to a subject comprises administering 1.0×10² to 1.0×10¹⁵, 1.0×10³ to 1.0×10¹⁴, 1.0×10⁴ to 1.0×10¹³, 1.0×10⁵ to 1.0×10¹², or 1.0×10⁶ to 1.0×10¹⁰ GC/kg to the subject. In some embodiments, a method for administering RIPs to a subject comprises administering 1.0×10² to 1.0×10¹⁵ GC/kg to the subject. In some embodiments, a method for administering RIPs to a subject comprises administering 1.0×10⁴ to 1.0×10¹³ GC/kg to the subject. In some embodiments, a method for administering RIPs to a subject comprises administering 1.0×10⁶ to 1.0×10¹² GC/kg to the subject. In some embodiments, a method for administering RIPs to a subject comprises administering 1.0×10⁹ to 1.0×10¹⁰ GC/kg to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, 1.0×10¹¹ infectious units to the subject. In some embodiments, a method for administering RIPs to a subject comprises administering 1.0×10² to 1.0×10⁵, 1.0×10³ to 1.0×10¹⁴, 1.0×10⁴ to 1.0×10¹³, 1.0×10⁵ to 1.0×10¹², or 1.0×10⁶ to 1.0×10¹⁰ infectious units to the subject. In some embodiments, the method for administering RIPs to a subject comprises administering 1.0×10⁵ to 1.0×10¹⁵ infectious units to the subject. In some embodiments, the method for administering RIPs to a subject comprises administering 1.0×10⁶ to 1.0×10¹³ infectious units to the subject. In some embodiments, the method for administering RIPs to a subject comprises administering 1.0×10⁷ to 1.0×10¹² infectious units to the subject. In some embodiments, the method for administering RIPs to a subject comprises administering 1.0×10⁹ to 1.0×10¹⁵ infectious units to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, 1.0×10¹¹ infectious units/kg to the subject. In some embodiments, a method for administering RIPs to a subject comprises administering 1.0×10² to 1.0×10¹⁵, 1.0×10³ to 1.0×10¹⁴, 1.0×10⁴ to 1.0×10¹³, 1.0×10⁵ to 1.0×10¹², or 1.0×10⁶ to 1.0×10¹⁰ infectious units/kg to the subject. In some embodiments, the method for administering RIPs to a subject comprises administering 1.0×10⁵ to 1.0×10¹⁵ infectious units/kg to the subject. In some embodiments, the method for administering RIPs to a subject comprises administering 1.0×10⁶ to 1.0×10¹³ infectious units/kg to the subject. In some embodiments, the method for administering RIPs to a subject comprises administering 1.0×10⁷ to 1.0×10¹² infectious units/kg to the subject. In some embodiments, the method for administering RIPs to a subject comprises administering 1.0×10⁹ to 1.0×10¹⁵ infectious units/kg to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, 1.0×10¹¹ PFU to the subject. In some embodiments, a method for administering RIPs to a subject comprises administering 1.0×10² to 1.0×10¹⁵, 1.0×10³ to 1.0×10¹⁴, 1.0×10⁴ to 1.0×10¹³, 1.0×10⁵ to 1.0×10¹², or 1.0×10⁶ to 1.0×10¹⁰ PFU to the subject. In some embodiments, the method for administering RIPs to a subject comprises administering 1.0×10⁵ to 1.0×10¹⁵ PFU to the subject. In some embodiments, the method for administering RIPs to a subject comprises administering 1.0×10⁶ to 1.0×10¹³ PFU to the subject. In some embodiments, the method for administering RIPs to a subject comprises administering 1.0×10⁷ to 1.0×10¹² PFU to the subject. In some embodiments, the method for administering RIPs to a subject comprises administering 1.0×10⁹ to 1.0×10¹⁵ PFU to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, 1.0×10¹¹ PFU/kg to the subject. In some embodiments, a method for administering RIPs to a subject comprises administering 1.0×10² to 1.0×10⁵, 1.0×10³ to 1.0×10¹⁴, 1.0×10⁴ to 1.0×10¹³, 1.0×10⁵ to 1.0×10¹², or 1.0×10⁶ to 1.0×10¹⁰ PFU/kg to the subject. In some embodiments, the method for administering RIPs to a subject comprises administering 1.0×10⁵ to 1.0×10¹⁵ PFU/kg to the subject. In some embodiments, the method for administering RIPs to a subject comprises administering 1.0×10⁶ to 1.0×10¹³ PFU/kg to the subject. In some embodiments, the method for administering RIPs to a subject comprises administering 1.0×10⁹ to 1.0×10¹⁵ PFU/kg to the subject. In some embodiments, the method for administering RIPs to a subject comprises administering 1.0×10⁷ to 1.0×10¹² PFU/kg to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, or 1.0×10¹¹ DU to the subject. In some embodiments, a method for administering RIPs to a subject comprises administering 1.0×10⁴ to 1.0×10⁵, 1.0×10⁵ to 1.0×10¹⁴, 1.0×10⁶ to 1.0×10¹³, 1.0×10⁷ to 1.0×10¹², or 1.0×10⁸ to 1.0×10¹² DU to the subject. In some embodiments, the method for administering RIPs to a subject comprises administering 1.0×10⁵ to 1.0×10¹⁴ DU to the subject. In some embodiments, the method for administering RIPs to a subject comprises administering 1.0×10⁶ to 1.0×10¹² DU to the subject. In some embodiments, the method for administering RIPs to a subject comprises administering 1.0×10⁹ to 1.0×10¹⁵ DU to the subject.

In some embodiments, a method for administering RIPs to a subject comprises administering at least 1.0×10⁴, 1.0×10⁵, 1.0×10⁶, 1.0×10⁷, 1.0×10⁸, 1.0×10⁹, 1.0×10¹⁰, or 1.0×10¹¹ DU/kg to the subject. In some embodiments, a method for administering RIPs to a subject comprises administering 1.0×10⁴ to 1.0×10⁵, 1.0×10⁵ to 1.0×10¹⁴, 1.0×10⁶ to 1.0×10¹³, 1.0×10⁷ to 1.0×10¹², or 1.0×10⁸ to 1.0×10¹² DU/kg to the subject. In some embodiments, the method for administering RIPs to a subject comprises administering 1.0×10⁵ to 1.0×10¹⁴ DU/kg to the subject. In some embodiments, the method for administering RIPs to a subject comprises administering 1.0×10⁶ to 1.0×10¹² DU/kg to the subject. In some embodiments, the method for administering RIPs to a subject comprises administering 1.0×10⁹ to 1.0×10¹⁵ DU/kg to the subject as per the body weight.

Quality control attributes can include purity and potency of the RIPs. The purity of RIPs can be determined using the ratio of the amount of protein from the host cells used to generate the of RIPs to the transducing units (amount host cell protein/TU). In some embodiments, the ratio of host cell protein to TUs can be 10, 5, 3, 2, or 1 ng or less host cell protein/TU or 750, 500, 400, 300, 200, 100, 50, 40, 30, 20, or 10 μg or less host cell protein/TU. In some embodiments, the ratio of host cell protein to TUs can be 1 ng or less host cell protein/TU. In some embodiments, the ratio of host cell protein to TUs can be 50 μg or less host cell protein/TU. In some embodiments, the ratio of host cell protein to TUs can be in the range of 10 to 1 ng host cell protein/TU. In some embodiments, the ratio of host cell protein to TUs can be in the range of 10 to 0.5, 10 to 1, 8 to 2, 6 to 3, or 5 to 3 ng host cell protein/TU. In some embodiments, the ratio of host cell protein to TUs can be in the range of 10 to 1 ng host cell protein/TU. In some embodiments, the ratio of host cell protein to TUs can be in the range of 8 to 2 ng host cell protein/TU. In some embodiments, the ratio of host cell protein to TUs can be in the range of 6 to 2 ng host cell protein/TU. In some embodiments, the ratio of host cell protein to TUs can be in the range of 750 to 10, 500 to 20, 400 to 30, 300 to 40, or 200 to 50 pg host cell protein/TU. In some embodiments, the ratio of host cell protein to TUs can be in the range of 500 to 20 pg host cell protein/TU. In some embodiments, the ratio of host cell protein to TUs can be in the range of 200 to 50 pg host cell protein/TU. In some embodiments, the host cell can be a HEK 293 cell line or variant thereof including a HEK 293T cell line. In some embodiments, the ratio of HEK protein to TUs can be 10, 5, 3, 2, or 1 ng or less HEK protein/TU or 750, 500, 400, 300, 200, 100, 50, 40, 30, 20, or 10 μg or less HEK protein/TU. In some embodiments, the ratio of HEK protein to TUs can be 1 ng or less protein/TU. In some embodiments, the ratio of HEK protein to TUs can be 50 μg or less HEK protein/TU.

The potency of RIPs present in a delivery solution or RIP formulation can be determined using the ratio of the TUs to the ng of p24 protein. In some embodiments, the ratio of TUs to the ng of p24 protein can be 100, 200, 300, 400, 500, 1,000, 4,000, 10,000, 12,500, or 15,000 or more TUs/ng of p24 protein. In some embodiments, the ratio of TUs to the ng of p24 protein can be 100 to 15,000, 500 to 12,500, or 1,000 to 10,000 TUs/ng of p24. In some embodiments, the ratio of TUs to the ng of p24 protein can be 100 to 15,000 TUs/ng of p24. In some embodiments, the ratio of TUs to the ng of p24 protein can be 1,000 to 10,000 TUs/ng of p24.

In some embodiments, a delivery solution or RIP formulation can include ratios of host cell protein/TU and TU/ng p24 protein being, respectively: 1 ng host cell protein/TU or less and 100 TU/ng p24 protein or more; 1 ng host cell protein/TU or less and 500 TU/ng p24 protein or more; 1 ng host cell protein/TU or less and 1,000 TU/ng p24 protein or more; 1 ng host cell protein/TU or less and 5,000 TU/ng p24 protein or more; 1 ng host cell protein/TU or less and 10,000 TU/ng p24 protein or more; 1 ng host cell protein/TU or less and 12,500 TU/ng p24 protein or more; 1 ng host cell protein/TU or less and 15,000 TU/ng p24 protein or more; 50 pg host cell protein/TU or less and 100 TU/ng p24 protein or more; 50 pg host cell protein/TU or less and 500 TU/ng p24 protein or more; 50 pg host cell protein/TU or less and 1,000 TU/ng p24 protein or more; 50 pg host cell protein/TU or less and 5,000 TU/ng p24 protein or more; 50 pg host cell protein/TU or less and 10,000 TU/ng p24 protein or more; 50 pg host cell protein/TU or less and 12,500 TU/ng p24 protein or more; or 50 pg host cell protein/TU or less and 15,000 TU/ng p24 protein or more.

In some embodiments, the host cell can be a HEK 293 cell line or variant thereof including a HEK 293T cell line. In such embodiments, a delivery solution or RIP formulation can include ratios of HEK protein/TU and TU/ng p24 protein being, respectively: 1 ng HEK protein/TU or less and 100 TU/ng p24 protein or more; 1 ng HEK protein/TU or less and 500 TU/ng p24 protein or more; 1 ng HEK protein/TU or less and 1,000 TU/ng p24 protein or more; 1 ng HEK protein/TU or less and 5,000 TU/ng p24 protein or more; 1 ng HEK protein/TU or less and 10,000 TU/ng p24 protein or more; 1 ng HEK protein/TU or less and 12,500 TU/ng p24 protein or more; 1 ng HEK protein/TU or less and 15,000 TU/ng p24 protein or more; 50 pg HEK protein/TU or less and 100 TU/ng p24 protein or more; 50 pg HEK protein/TU or less and 500 TU/ng p24 protein or more; 50 pg HEK protein/TU or less and 1,000 TU/ng p24 protein or more; 50 pg HEK protein/TU or less and 5,000 TU/ng p24 protein or more; 50 pg HEK protein/TU or less and 10,000 TU/ng p24 protein or more; 50 pg HEK protein/TU or less and 12,500 TU/ng p24 protein or more; or 50 pg HEK protein/TU or less and 15,000 TU/ng p24 protein or more.

In some embodiments, the concentration of RIPs present in a delivery solution and/or RIP formulation can be at least 1×10⁶, 5×10⁶, 1×10⁷, 5×10⁷, 1×10⁸, 2×10⁸, 5×10⁸, or 1×10⁹ TU/ml. In some embodiments, the concentration of RIPs can be 1×10⁶ to 1×10⁷, 1×10⁶ t 1×10⁸, 1×10⁶ to 1×10⁹ TU/ml, 1×10⁷ to 1×10⁹ TU/ml, or 1×10⁸ to 1×10⁹ TU/ml.

In some embodiments, a delivery solution and/or cell formulation provided herein comprises an aggregating agent as provided herein. In some embodiment, a delivery solution and/or cell formulation comprises a cellular matrix, such as a hyaluronic acid matrix and/or a collagen matrix. Such cell formulation can be an ex vivo cell formulation or an in vivo cell formulation localized within a muscle or subcutaneously within a subject. In illustrative embodiments, the hyaluronic acid and/or collagen matrix are localized subcutaneously and in some embodiments, such matrix is the natural subcutaneous matrix found in the subject. Such a matrix found or localized subcutaneously in a subject when including exogenous lymphocytes such as tumor infiltrating lymphocytes and/or modified lymphocytes as provided herein, optionally including other cell formulation components provided herein, can be considered an artificial lymph node. As such, methods provided herein for administering cell formulations to a subject subcutaneously, where the cell formulations comprise an aggregating agent and/or a cellular matrix and/or where a matrix comprising only a subject's natural matrix components is formed around modified lymphocytes delivered subcutaneously, can be referred to as methods for forming an artificial lymph node, and such resulting structures can be considered artificial lymph nodes. In some embodiments the composition comprises modified T cells and/or NK cells, and/or TILs in an artificial matrix, such as a hyaluronic acid and/or collagen matrix, that is located subcutaneous.

In some embodiments, the unwanted cells can be epitope-masking target cells that express both a CAR and the antigen the CAR binds to. In some embodiments, the epitope-masking target cells can be depleted, removed, or killed by contacting them with CAR-T cells expressing a CAR to a different epitope or antigen that the target cells do not mask in a method provided herein, after genetically modifying the cells using methods provided herein. Such first CAR and second CAR in these embodiments, can be referred to as a CAR-pair. In some embodiments, cells expressing two or more separate CARs, and in illustrative embodiments two CARs expressed in two populations of cells, can be used to kill the epitope-masking target cells that are masking only one of the epitopes. In some embodiments, the two populations of cells are transduced or transfected separately so each population expresses either a first CAR or a second CAR. In illustrative embodiments, the epitope-masking target cell expressing the first or second CAR does not mask the epitope that the second and first CAR, respectively, bind to. In some embodiments, the first and second CARs can bind to different epitopes of the same antigen expressed on the epitope-masking target cell. In other embodiments, the first and second CARs can bind to different antigens expressed on the same epitope-masking target cell, including any of the antigens disclosed elsewhere herein. In some embodiments, the first and second CARs can bind to different epitopes of, or different antigens selected from CD19, CD20, CD22, CD25, CD32, CD34, CD38, CD123, BCMA, TACI, or TIM3. In some embodiments, two containers containing separate polynucleotides, each of which encodes one of the CARs of a CAR pair directed to two different epitopes or antigens expressed on the same target cell, are provided in kits herein. In other embodiments, one CAR can be an extracellular ligand or receptor binding to a cancer antigen and the other can be a CAR derived from an antibody fragment. In other embodiments both CARs can be an extracellular ligand or receptor against a different cancer antigen. In one example the CAR is BCMA and April is the ligand binding protein to TACI and BCMA receptors. In further illustrative embodiments, the first CAR can bind to CD19 and the second CAR can bind to CD22, both of which are expressed on B cells and lymphomas. In illustrative embodiments, the modified cell population expressing the first CAR and the modified cell population expressing the second CAR are formulated separately. In some embodiments, the separate cell formulations are introduced or reintroduced back into the subject at different sites. In some embodiments, separate cell formulations are separately introduced or reintroduced back into the subject at the same site. In other embodiments, the modified cell populations are combined into one formulation that is optionally introduced or reintroduced back into the subject. In illustrative embodiments wherein the cell populations are combined, the cell populations are not combined until after a washing step in which the cells are washed away from the recombinant nucleic acid vectors.

In some embodiments of any of the aspects herein that include a modified or genetically modified T cell or NK cell, or a kit or composition for producing the same, the proliferation and survival of genetically modified T cells and/or NK cells expressing a CAR can be promoted by adding an antigen to which an ASTR of a CAR binds, to a composition, such as a cell formulation, or environment, such as a subcutaneous environment or an intramuscular environment, comprising the genetically modified T cells and/or NK cells. In certain illustrative embodiments, the genetically modified T cell and/or NK cells are genetically modified with a nucleic acid encoding a CAR, but not with a nucleic acid encoding an LE. In some embodiments, the antigen can be added to a cell formulation comprising, or co-administered with, modified and/or genetically modified T cells and/or NK cells in cell formulations and methods provided herein. In some embodiments, the antigen is a protein antigen. In some embodiments, the antigen is mRNA encoding the protein antigen. In some embodiments, the antigen can be soluble. In some embodiments, the antigen can be immobilized on a surface of an artificial matrix, such as a hydrogel. In illustrative embodiments, the antigen can be expressed on the surface of a target cell. In some embodiments, such target cells are present in large numbers in whole blood and are naturally present in the cell formulation without having to be added. In some embodiments, B cells present in whole blood, isolated TNCs, and isolated PBMCs naturally present in the cell formulation can be target cells for T cells and/or NK cells expressing a CAR directed to CD19 or CD22, which are both expressed on B cells. In other embodiments, such target cells are not present in whole blood or are not present in large numbers in whole blood and need to be added exogenously to a cell formulation provided herein. In some embodiments, target cells can be isolated or enriched from a subject, such as from a tumor sample, using methods known in the art. In other embodiments, cells from the subject are modified to express a target antigen. In illustrative embodiments, the antigen expressed on the target cell can include all or a portion of the protein that contains the antigen. In further illustrative embodiments, the antigen expressed on the target cell can include all or a portion of the extracellular domain of the protein that includes the antigen. In some embodiments, the antigen expressed on the target cell can be a fusion with a transmembrane domain that anchors it to the cell surface. In some embodiments, any of the transmembrane domains disclosed elsewhere herein can be used. In some embodiments, the antigen expressed on the target cell can be a fusion with a stalk domain. In some embodiments, any of the stalk domains disclosed elsewhere herein can be used. In illustrative embodiments, the antigen can be a fusion with a CD8 stalk and transmembrane domain (SEQ ID NO:24).

In some embodiments, cells in a first cell mixture, and in illustrative embodiments cells in a first cell mixture from the subject, are modified with a recombinant nucleic acid vector encoding an antigen, and cells in a separate second cell mixture from a subject, and in illustrative embodiments cells in a second mixture from the same subject, are modified to express a CAR that binds the antigen. In further illustrative embodiments, either or both of the cell mixtures is whole blood, isolated TNCs, or isolated PBMCs. In illustrative embodiments, the first cell mixture can be modified with a recombinant nucleic acid vector encoding a fusion protein of the extracellular domain of Her2 and the transmembrane domain of PDGF and the second cell mixture can be modified with a recombinant nucleic acid vector encoding a CAR directed to HER2. The cells can then be formulated into a delivery solution to form a cell formulation. Thus, in one aspect, provided herein is a pair of such cell mixtures, or a pair of cell formulations, each comprising one of the cell mixtures or cell formulations, typically physically separated in any of the vessels such as cell bags, provided herein for holding cell formulations. Optionally, the cell formulations are administered to the subject at varying CAR effector cell-to-target-cell ratios. In some embodiments, the effector-to-target ratio at the time of formulation or administration is or is about 10:1, about 9:1, about 8:1, about 7:1, about 6:1, about 5:1, about 4:1, about 3:1, about 2:1, about 1:1, about 1:2, about 1:3, about 1:5, about 1:6, about 1:7, about 1:8, about 1:9, or about 1:10. In illustrative embodiments, antigen is co-administered with the modified T and/or NK cells subcutaneously or intramuscularly.

In some embodiments of any of the aspects herein that include a modified or genetically modified T cell or NK cell, or methods, compositions, and kits for genetically modifying T cells and/or NK cells, the proliferation and survival of genetically modified T cells and/or NK cells expressing a CAR can be promoted by cross-linking CAR molecule within a genetically modified T cell or NK cell, in the absence of the CAR molecules binding to their cognate antigens. Thus, in some embodiments, a T cell or NK cell can comprise an epitope tag bound by an antibody and cross-linked to an epitope tag of a second CAR on the same T cell or NK cell. In some embodiments, the extracellular domain of the CAR can include the epitope tag. In illustrative embodiments, the epitope tag can be in the stalk domain. In some embodiments, the epitope tag can be His5 (HHHHH; SEQ ID NO:76), HisX6 (HHHHHH; SEQ ID NO:77), c-myc (EQKLISEEDL; SEQ ID NO:75), Flag (DYKDDDDK; SEQ ID NO:74), Strep Tag (WSHPQFEK; SEQ ID NO:78), HA Tag (YPYDVPDYA; SEQ ID NO:73), RYIRS (SEQ ID NO:79), Phe-His-His-Thr (SEQ ID NO:80), or WEAAAREACCRECCARA (SEQ ID NO:81). In illustrative embodiments, the epitope tag can be the HisX6 tag (SEQ ID NO:77). In some embodiments, the CARs can be cross-linked and activated by adding soluble antibodies or antibody mimetics that bind the epitope tag, or in illustrative embodiments by adding cells, also referred to herein as universal feeder cells, expressing antibodies or antibody mimetics on their surfaces that bind the epitope tag. In some embodiments, the same universal feeder cells, for example universal feeder cells expressing an anti-HisX6 antibody, can be used with cells that express CARs that bind to different antigens but that include the same epitope tag, for example HisX6. In some embodiments, the CARs can be cross-linked and activated by adding mRNA that encodes for one or more antibodies or antibody mimetics that bind the epitope tag. The mRNA may encode for antibodies or antibody mimetics that are soluble, membrane-bound, or both soluble and membrane-bound in some embodiments.

The following non-limiting examples are provided purely by way of illustration of exemplary embodiments, and in no way limit the scope and spirit of the present disclosure. Furthermore, it is to be understood that any inventions disclosed or claimed herein encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more ofthe reagents disclosed herein may be used in the method, unless one of ordinary skill in the art would understand otherwise.

### EXAMPLES

### Example 1. Materials and Methods for Transduction Experiments

This Example provides materials and methods used in experiments disclosed in subsequent Examples herein.

Recombinant lentiviral particle production by transient transfection.

HEK 293T cells (Lenti-X™ 293T, Clontech) were adapted to chemically defined suspension culture by serial expansion in Freestyle™ 293 Expression Medium (animal origin-free, chemically defined, and protein-free), (ThermoFisher Scientific) followed by repeated single cell cloning by serial dilution in 96 well plates to generate a master and working cell bank of cells named F1XT cells, and were used as the packaging cells for experiments herein unless noted otherwise.

Where noted, a typical 4 vector packaging system included 3 packaging plasmids that encoded (i) gag/pol, (ii) rev, and (iii) a pseudotyping element such as VSV-G. The 4th vector of this packaging system is the genomic plasmid, a third generation lentiviral expression vector (containing a deletion in the 3′ LTR leading to self-inactivation) that encoded 1 or more genes of interest. For transfections using 4 plasmids, the total DNA used (1 pg/mL of culture volume) was a mixture of the 4 plasmids at the following molar ratios: 1× gag/pol-containing plasmid, 1×Rev-containing plasmid, 1× viral envelope containing plasmid (VSV-G unless noted otherwise), and 2× genomic plasmid unless noted otherwise. Where noted, a 5 vector packaging system was used to generate lentiviral particles. The system included 3 packaging plasmids that encoded (i) gag/pol, (ii) rev, and (iii) a pseudotyping element such as VSV-G. The 4^{th} vector of this packaging system was the genomic plasmid, a third generation lentiviral expression vector (containing a deletion in the 3′ LTR leading to self-inactivation) that encoded 1 or more genes of interest. The 5^{th} vector encoded the T cell activation element antiCD3-scFvFc-GPI. For transfections, the total DNA used (1 pg/mL of culture volume) was a mixture of the 5 plasmids at the following molar ratios: 1× gag/pol-containing plasmid, 1× Rev-containing plasmid, 1×VSV-G containing plasmid, 2× genomic plasmid, and 1× of the 5^{th} vector unless noted otherwise.

For small-scale (3 ml) lentivirus production, plasmid DNA was dissolved in either 1.5 ml Gibco™ Opti-MEM™ growth media for every 30 mL of culture containing packaging cells in Freestyle™ 293 Expression Medium. Polyethylenimine (PEI) (Polysciences) (dissolved in weak acid) was diluted in 1.5 ml Gibco™ Opti-MEM™ to 2 μg/ml. A 3 ml mixture of PEI and DNA was made by combining the two prepared reagents at a ratio of 2 ug of PEI to 1 ug of DNA. After a 5-minute room temperature incubation, the two solutions were mixed together thoroughly, and incubated at room temperature for 20 more minutes. The final volume (3 ml) was added to 30 ml of packaging cells in suspension at 1×10⁶ cells/ml in a 125 ml Erlenmeyer flask. The cells were then incubated at 37° C. for 72 hours with rotation at 125 rpm and with 8% CO₂ for transfection. For larger-scale lentivirus production (6.6 to 10 L), volumes and ratios of reagents were increased proportionally to support transfection and fermentation in larger reactors of F1XT cells that had been expanded through Erlenmeyer flasks of increasing size until final reactor inoculation and addition of transfection material when cells had reached 1×10⁶ cells/ml. Retroviral particles made by all of these methods are free of non-human derived animal proteins.

After 72 hours, for small scale lentivirus production, the supernatants were harvested and clarified by centrifugation at 1,200 g for 10 minutes. The clarified supernatants were sterile-filtered into anew container. Substantially purified virus was obtained from these clarified supernatants by addition of polyethylene glycol (PEG) followed by centrifugation. For PEG precipitation, % volume PEG (Takara Lenti-X™ Concentrator) was added to the clarified supernatant and incubated overnight at 4° C. The mixture was then centrifuged at 1600 g for 1 hour (for 50 ml conical tubes) or 1800 g for 1.5 hours (for 500 ml conical tubes). The supernatant was discarded, and the lentiviral particle pellets were resuspended in 1:100 of the initial volume of packaging cell culture.

For larger scale purification by depth filtration, culture media was harvested 72 hours after addition of transfection solution and clarified by depth filtration using Sartorius (#5445306G9 or #5445306G8) or Millipore (#MCE50027H1) depth filter cartridges using a peristaltic pump. Clarified media was then concentrated using a 500 Kd mPES Hollow Fiber TFF Module (Spectrum) on a KrossFlow TFF System (Spectrum) with a TMP of 2.0+/−0.5 PSI. Following addition of MgCl₂ to 2 mM final volume, Benzonase (EMD Millipore) was added to 50 U/ml to fragment residual DNA. The concentrate was then recirculated followed by diafiltration using 10 volumes of PBS 4% Lactose. The substantially purified concentrated and formulated virus was then sterile-filtered and frozen for use. In other cases, the benzonase was added first to the culture media 24 hours post transfection and the post depth-filtered material was diluted with concentrated Tris NaCl to 50 mM Tris 300 mM NaCl pH 8.0 final. Following loading on a Mustang-Q resin (Pall) and elution with 2 M NaCl, the virus was diluted with PBS Lactose and processed by TFF per above.

Lentiviral particles were titered by serial dilution and analysis of transgene expression, by transduction into Jurkat cells and analysis of transgene expression by FACS or qPCR for lentiviral genome using Lenti-X™ qRT-PCR Titration Kit (#631235) or p24 assay ELISA kit from Takara (Lenti-X™ p24 Rapid Titer Kit #632200). Copy number was calibrated against a plasmid standard containing target sequences for lentivirus and human RNAseP.

### Genomic Plasmids Used in Examples.

The following lentiviral genomic vectors encode genes and features of interest as indicated:

GCAR-19 encodes a CD19 CAR and a polypeptide lymphoproliferative element comprised of a FLAG-tagged anti-CD19scFv, a CD8 stalk and transmembrane region, and an intracellular domain from CD3z followed by T2A and an eTagged lymphoproliferative (anti-CD19:CD8:CD3z-T2A-LE)

F1-3-22 encodes a second generation CD19 CAR comprised of an anti-CD19scFv, a CD8 stalk and transmembrane region, a CD137 intracellular domain, and an intracellular domain from CD3z followed by T2A and an eTag (anti-CD19:CD8:CD137:CD3z-T2A-eTag).

F1-3-23 encodes a CD19 CAR comprised of an anti-CD19scFv, a CD8 stalk and transmembrane region, and an intracellular domain from CD3z followed by T2A and an eTag (anti-CD19:CD8:CD3z-T2A-eTag).

F1-3-469 encodes a CD19 CAR and a polypeptide lymphoproliferative element comprised of a FLAG-TAG (DYKDDDDK; SEQ ID NO:74), a linker, an anti-CD19scFv, a CD8 stalk, a transmembrane region, and an intracellular domain from CD3z followed by T2A and lymphoproliferative element comprising an extracellular domain containing a variant of c-Jun including a leucine zipper motif and an eTAG, a transmembrane domain, and a synthetic intracellular domain that is capable of associating with signaling molecules that include Jaks, STATs, TRAFs, and Shc.

All mice used in the examples were handled in accordance with Institutional Animal Care and Use committee approved protocols.

### Example 2. Administration of Substantially Pure Recombinant Retroviral Particles Directly to Humanized Lymphoreplete Hosts Results in Effective T Cell Transduction and Effector Function In Vivo

In this Example, substantially pure recombinant retroviral particles (such as, RIP formulations) were injected directly into several lymphoreplete mouse models. The recombinant retroviral particles were pseudotyped with VSV-G, displayed an activation element on their surface, and encoded a CD19 CAR and a lymphoproliferative element. Following perilymphatic administration of the recombinant retroviral particles (RIP), the mice were assessed for the presence of CAR transduced T cells and the presence of CAR target cells.

Recombinant lentiviral particles encoding GCAR-19 pseudotyped with VSV-G and displaying the T cell activation element, UCHT1-scFvFc-GPI (GCAR-19GU) (RIP) were produced by transfecting F1XT cells using the 5 plasmid protocol using a 10 L liter scale and purified by a combination of depth filtration, TFF, benzonase treatment, diafiltration, and formulated to generate substantially pure viral particles free of non-human animal proteins as described in Example 1. The GCAR-19 recombinant retroviral particles (RIP) were formulated (RIP formulation) and frozen in PBS 4% Lactose with lots demonstrating potency of 2-5×10⁷ TU/ml based upon a qPCR endpoint titer assay or flow cytometry.

Two different models of humanized lymphoreplete hosts were used in this Example. One was NSG-MHC1/2-DKO (NSG-(K^{b}D^{b})^{null}(IA)^{null} PBMC-humanized mice. These mice combine the features of the highly immunodeficient NSG mouse with MHC class 1 deficiency and MHC class 2 deficiency and were reconstituted with a human immune system by injecting 200 μl of human PBMCs at 5.0×10⁷ cells/mL in PBS-HSA (10 million PBMCs) IV in the tail vein. The other model was NSG-SGM3 CD34-humanized mice. These mice combine the features of the highly immunodeficient NSG mouse with transgenic overexpression of SCF, GM-CSF, and IL-3 which promote the proliferation and differentiation to immune cells, of the human CD34 hematopoietic stem cells that were transferred following whole body irradiation of these mice. The NSG-MHC1/2-DKO (NSG-(K^{b}D^{b})^{null}(IA)^{null} PBMC-humanized model transfers cells with a donor derived TCR profile but are resistant to graft versus host disease, whereas the CD34 model enables modification of human T cells that have differentiated in NSG mice from CD34 stem cells against a mixture of human and mouse MHC in vivo.

7 week-old NSG-MHC1/2-DKO PBMC-humanized mice (n=5) and 7 week-old NSG-SGM3 CD34-humanized mice (n=5) were injected intraperitoneally with 2×10⁷ TU (350 μl) of GCAR-19GU. Blood from each mouse was collected 5, 12, 18 (or 19), and 27 (or 28) days after dosing. The PBMCs were stained for FLAG, hCD3, and hCD20, and analyzed by FACS.

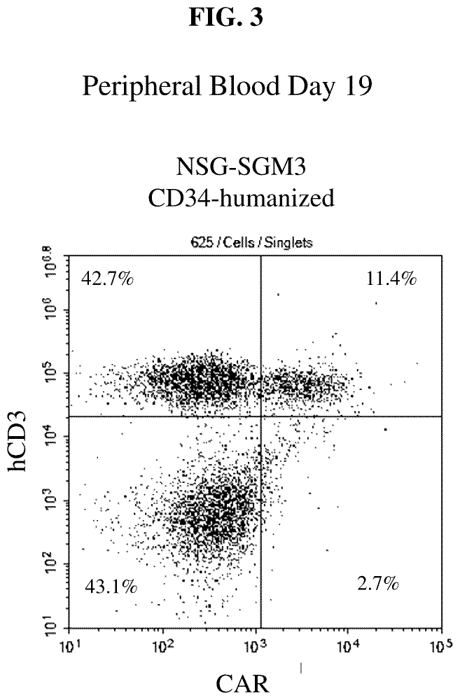

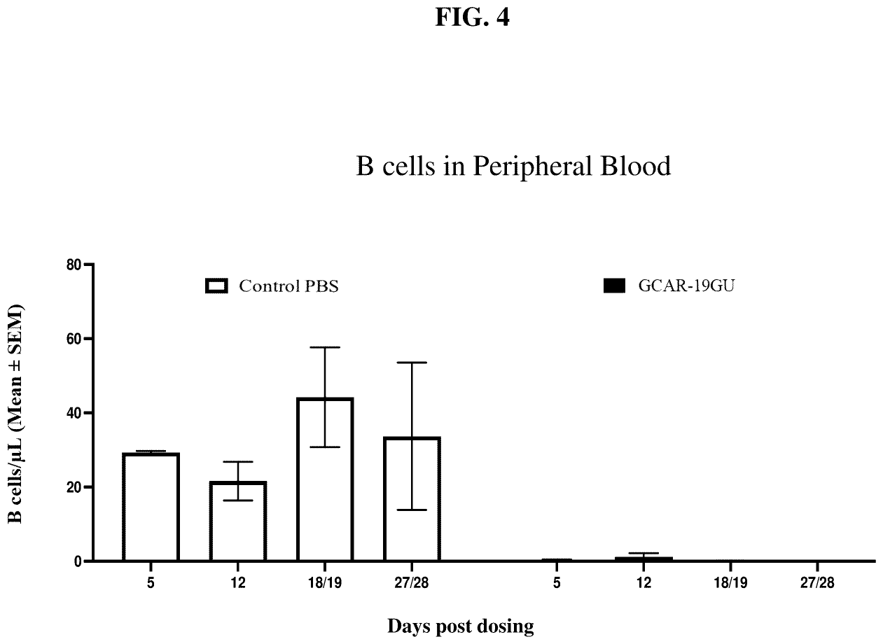

Expansion of CAR positive cells was observed in the periphery of NSG-SGM CD34-humanized mice. The greatest numbers of CAR-T cells in the peripheral blood was seen 19 days after dosing. A representative FACS plot in which 11.4% of the PBMCs were hCD3+ CAR+ CAR-T cells is shown in FIG. 3. The NSG-SGM3 CD34-humanized model contains high levels of human B cells. B cells per μl blood (as determined by CD20 expression) is shown in FIG. 4. Control mice mock treated with PBS contained high levels B cells in the periphery throughout the 28 day study, but mice treated with GCAR-19GU by IP injection exhibited an early and sustained absence of CD20 positive B cells. These data indicate that injection of GCAR-19GU leads to the in vivo modification of human CD3 positive T cells and the generation of CD3 positive CAR positive cells in lymphoreplete hosts. These CAR-T cells were functionally active and could kill their CD19-expressing B cell targets.

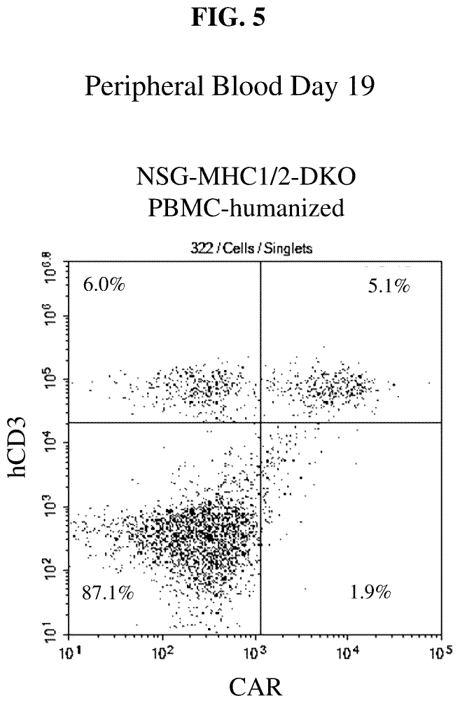

Expansion of CAR positive cells was also observed in the periphery of NSG-MHC1/2-DKO (NSG-(K^{b}D^{b})^{null}(IA)^{null} PBMC-humanized mice that were first administered human PBMC followed by the injection of a formulation comprising GCAR19GU RIP. While absolute B cell counts in peripheral blood were lower in this model, effective expansion of CD3 positive CAR positive cells was also observed by flow cytometry. A representative FACS plot in which 5.1% of the PBMCs were hCD3+ CAR+ CAR-T cells is shown in FIG. 5.

This example demonstrates that lentiviral particles displaying an activation element on their surface, and encoding a CAR and lymphoproliferative element (such as, RIP in a RIP formulation) when administered directly to a lymphoreplete host by perilymphatic delivery, can transduce/modify T cells in vivo and generate CAR-T cells in vivo that are functionally active. These results suggest that the vectors disclosed herein (RIP), and the formulation and/or delivery solution comprising RIP, such as RIP formulation may be used in CAR-T therapies by direct administration to patients without the need for ex vivo cell manufacturing or lymphodepleting chemotherapy. The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

### Example 3. CAR Cells that Also Express a Lymphoproliferative Element can Exhibit Sustained Cell Expansion without Exhaustion and Take on a T and NK Cell-Like Phenotype Following Serial Tumor Antigen Challenge In Vitro

In this Example, PBMCs transduced using a traditionally-based protocol with either a vector encoding a first generation CAR directed to CD19 or a vector encoding the same CAR and a lymphoproliferative element, were cultured in vitro and fed with CAR target cells to provide stimulation at regular intervals. The cultured cells were compared for the ability to expand and their expression of CD56 and markers of exhaustion.

The constructs used in this Example were F1-3-23 and F1-3-469. Briefly, the constructs encoded the same first generation CAR, comprised of an anti-CD19scFv, a CD8 stalk and transmembrane region, and an intracellular domain from CD3z, which was driven by the EF1-α promoter which is constitutively active in T and NK cells. F1-3-23 and F1-3-469 differed in that F1-3-469 also encoded a lymphoproliferative element.

Recombinant lentiviral particles were produced by transient transfection of 30 ml of F1XT using a 4 vector packaging system and purified by PEG precipitation as described in Example 1. Each sample was resuspended in 0.3 ml PBS with 3 mg/ml HSA.

The ability of the cells to proliferate and/or survive was examined in a first experiment. On Day 0, PBMCs from a single donor were enriched from buffy coats (San Diego Blood Bank) by density gradient centrifugation with Ficoll-Paque PREMIUM® (GE Healthcare Life Sciences) according to the manufacturer's instructions followed by lysis of red blood cells. 1.5×10⁶ viable PBMCs were seeded in the wells of G-Rex 6 Well Plates (Wilson Wolf, 80240M) in 3 ml Complete OpTmizer™ CTS T-Cell Expansion SFM supplemented with 100 IU/ml (IL-2), 10 ng/ml IL-7, and 50 ng/ml anti-CD3 antibody (317326, Biolegend) to activate the PBMCs for viral transduction. After incubation overnight at 37° C. and 5% CO₂, lentiviral particles including the constructs described above were added directly to the activated PBMCs at an MOI of 5 and incubated overnight at 37° C. and 5% CO₂. The following day, the media volume in each well was brought to 30 ml with Complete OpTmizer™ CTS™ T-Cell Expansion SFM and the plates were returned to the incubator.

The cells from each well were collected on Day 7, washed, and reseeded in the wells of G-Rex 24 Well Plates at 0.5×10⁶ cells in 1 ml of Complete OpTmizer™ CTS™ T-Cell Expansion SFM. 1×10⁶ Raji, which express CD19 that is recognized by the CD19 CAR, were added to samples designated as “fed” or no Raji cells were added to the samples designated as “unfed.” The volume in each well was brought up to 7 ml with Complete OpTmizer™ CTS™ T-Cell Expansion SFM. No IL-2, IL-7, or other exogenous cytokine was added at this or subsequent cell culture steps. Raji cells were added to the fed transduced PBMC samples every other day until Day 15 by removing 3 ml of media and replacing it with fresh media containing 1×10⁶ Raji cells. The cell density of transduced PBMCs was very high on Day 15 so the feeding protocol was modified. Starting on Day 15, 1.0×10⁶ CAR+ cells were reseeded into wells of new G-Rex 24 Well Plates, 1×10⁶ Raji cells were added, and the volume was brought to 7 ml with Complete OpTmizer™ CTS™ T-Cell Expansion Media.

Surface expression of CD56 and the exhaustion markers PD-1, LAG3, and TIM-3 were examined in a second experiment. A similar protocol was used to isolate, transduce, and culture PBMCs as described for the first experiment with the following differences; the starting material was an apheresis sample, anti-CD3 antibody and 100 IU/ml IL, but no IL-7 was used to activate the cells, and the transduced cells were not fed Raji cells, but rather were fed donor-matched PBMCs on Days 7, 14, and 21.

To analyze the cells in each experiment, 100 μl or 150 μl of cells were removed at each time point and stained with antibodies to CD3, CD8, CD19 CAR, CD56, LAG3, PD-1, and/or Tim-3. Flow cytometry was used to count the total live cells, CD3+CD19 CAR+, CD8+CD56+, CD8+LAG3+, CD3+PD-1+, and CD3+Tim-3CD8+ cells.

### Results

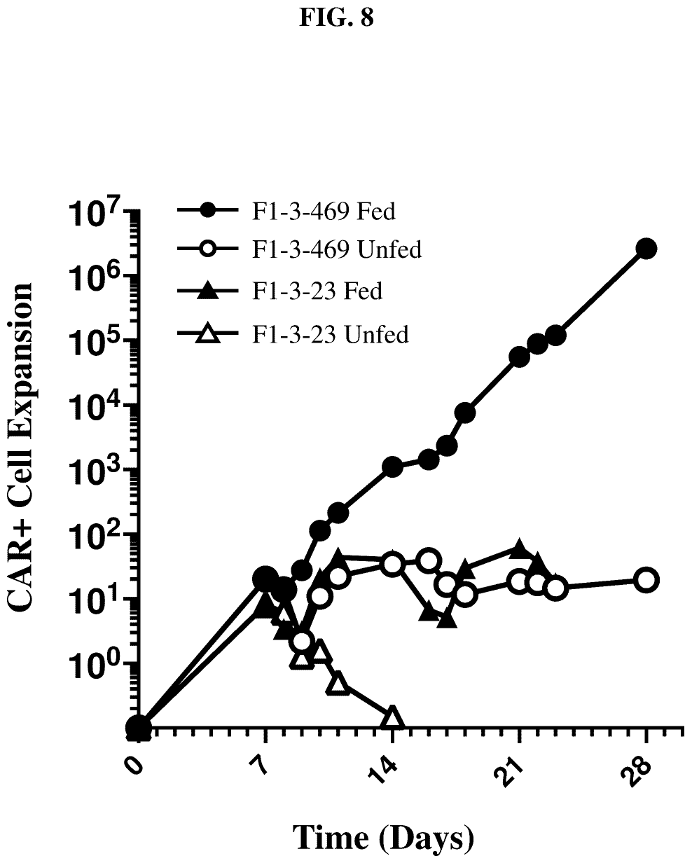

The results of the first experiment are shown in FIG. 8. Following cytokine withdrawal on Day 7, PBMCs modified with F1-3-469, which included a lymphoproliferative element, exhibited expansion when fed Raji cells. In the absence of Raji cells, F1-3-469 modified PBMCs were able to maintain a steady number for 28 days. In contrast, PBMCs modified with F1-3-23 were only able to maintain a steady number in the presence of Raji cells. In the absence of Raji cells, F1-3-23 modified cells were unable to survive in culture.

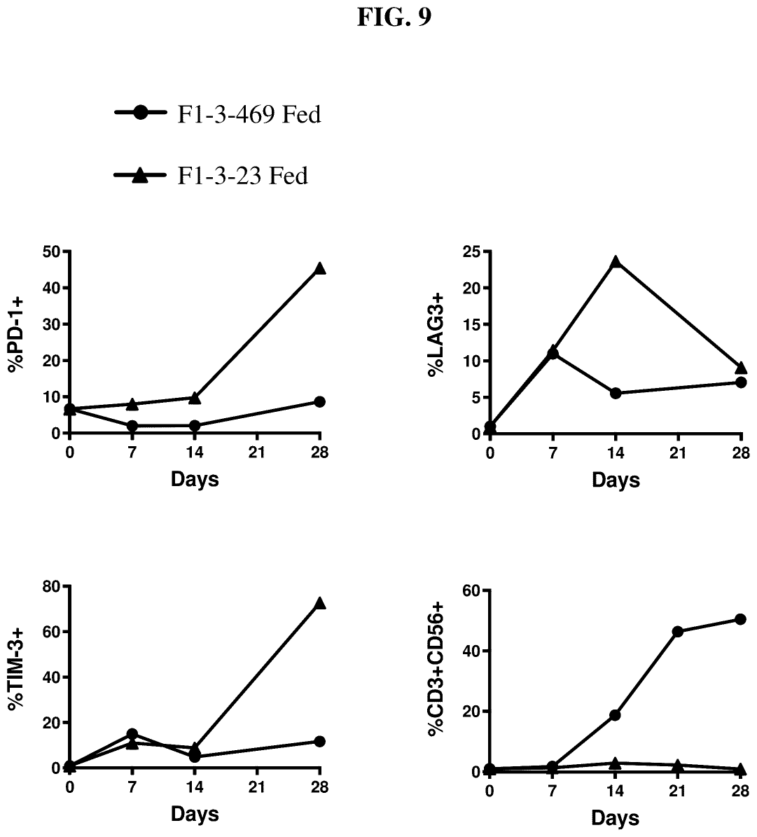

The results of the second experiment are shown in FIG. 9. Surprisingly, following serial Raji tumor exposure, CD8+F1-3-469 modified PBMCs exhibited reduced expression of the exhaustion markers PD-1, LAG3, and TIM-3 when the lymphoproliferative element. The presence of the lymphoproliferative element also facilitates conversion of these modified cells to CD56 positivity during continued CAR-target cell exposure.

This Example demonstrates that lymphoproliferative elements as disclosed herein are capable of promoting the survival and proliferation of modified cells and promoting the conversion of T cells to cells that express CD56 and display reduced markers of cellular exhaustion.

### Example 4. T Cells Genetically Modified with a Vector Encoding a CAR and a Lymphoproliferative Element Differentiate into CAR-TaNKs when Cultured In Vitro

In this Example, CD3+CD56− PMBCs genetically modified with a vector encoding a CD19 CAR and an LE were cultured in vitro in the presence of IL-2 for 14 days and examined by flow cytometry for CAR expression and markers of T and NK cells.

Fresh human PBMCs that had been magnetically sorted for CD3+CD56− cells were obtained from Oribiotech. 1.1×10⁷ viable PBMCs were seeded in the wells of G-Rex 6 Well Plates at 1×10⁶ cells/ml in RPMI1640 supplemented with 10% FBS and 1% PS (Complete RPMI). F1-3-469GU viral particles were added to the PBMCs at an MOI of 5. The transduction reaction mixtures were incubated for 4 hours in a standard humidified tissue culture incubator at 37° C. and 5% CO₂. After the 4 hour exposure, the cells were pelleted and washed 3 times in Complete RPMI. 3.0×10⁶ genetically modified PBMCs were seeded in triplicate in the wells of G-Rex 6 Well Plates in 8 mls of Complete RPMI+10 ng/ml rhIL-2. The cells were cultured for 14 days with half the volume of media and IL-2 exchanged on Days 2, 4, 8, 10, and 11. On Day 14, the cells were harvested and stained for CD3, FLAG, CD56, CD57, and NKG2D and analyzed by FACS.

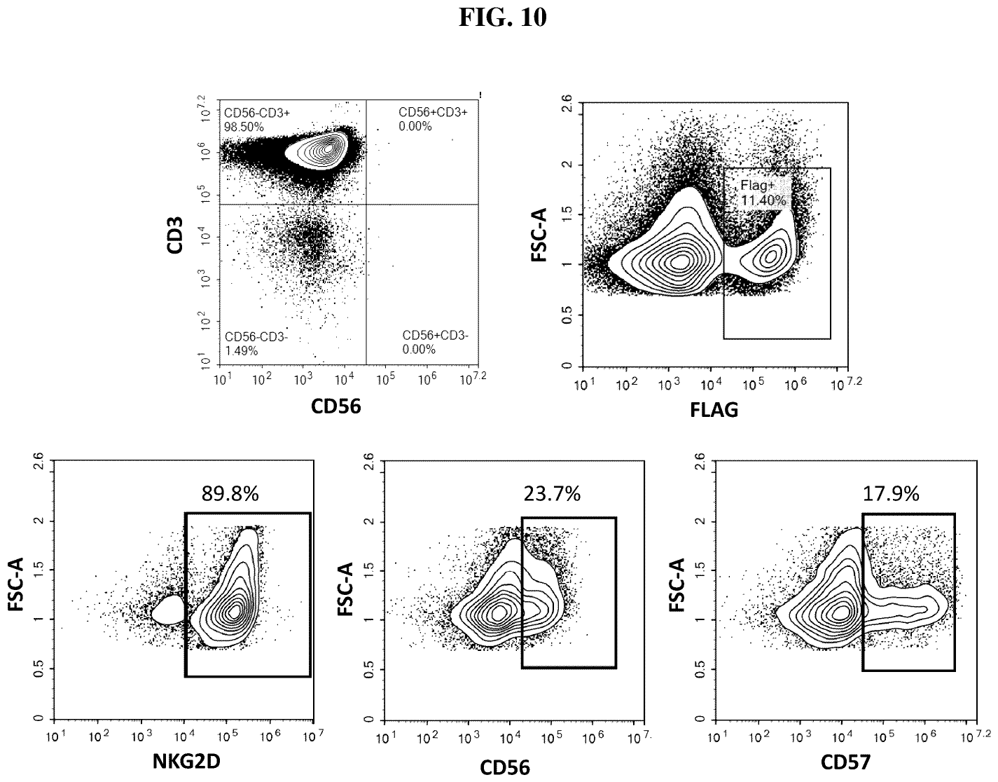

As shown in FIG. 10, 98.5% of the starting population of PBMCs were CD3+CD56−. After transduction and 14 days in culture, 11.4% of the cells expressed the CAR (as determined by FLAG). Among these CAR positive cells, 89.8% expressed the activation marker NKG2D. Among these CAR positive cells, 23.7% and 17.9% of the cells expressed CD56 and CD57, respectively.

This example shows that T cells which lack CD56 expression, when genetically modified with a vector encoding a CAR and an LE can differentiate into CAR-TaNKs with a T and NK-like phenotype that includes expression of NKG2D, CD56, and CD57. Therefore, while a starting population of PBMCs that express NKG2D, CD56, and or CD57 may be genetically modified by the vector and subsequently expand, a significant population of genetically modified NKG2D+, CD56+, and/or CD57+ PMBCs started as NKG2D−, CD56−, and/or CD57− and differentiate to express one or more of these markers.

### Example 5. PBMCs Genetically Modified with a Vector Encoding a CAR and a Lymphoproliferative Element Engraft and Differentiate into CAR-TaNKs in Lymphoreplete Hosts

In this example, a cell formulation comprising PBMCs genetically modified to express CARs were introduced into lymphoreplete hosts. The PBMCs were modified by a 4-hour incubation with RIPs encoding a CAR and a lymphoproliferative element, and the phenotype of the CAR cells was examined 28 days after dosing. For comparison, CAR cells were made using a more traditional method of PBMC transduction with RIPs encoding a second generation CAR and no lymphoproliferative element, expanded ex vivo, introduced into lymphoreplete mice, and the phenotype of the CAR cells was also examined 28 days after dosing.

Fresh PBMCs were modified with F1-3-469GU using a 4 hour rPOC cell process to obtain a cell formulation consisting of modified cells and injected subcutaneously were compared to PBMCs transduced with F1-3-22 using the more traditional cell process involving overnight activation with IL-2, IL-7, and anti-CD3/anti-CD28 conjugated beads, overnight transduction, and 12 days of ex vivo expansion, followed by injection IV.

Seven week-old NSG-MHC1/2-DKO mice were reconstituted with human PBMCs on day 0 by injecting 200 μl of human PBMCs at 5.0×10⁷ cells/mL in PBS-HSA (10 million PBMCs) IV in the tail vein. On day 2, mice were dosed subcutaneously with 1×10⁶ PBMCs modified with F1-3-469GU. On day 14, the PBMCs modified previously with F1-3-22 were thawed and mice were dosed intravenously with 1×10⁶ cells.

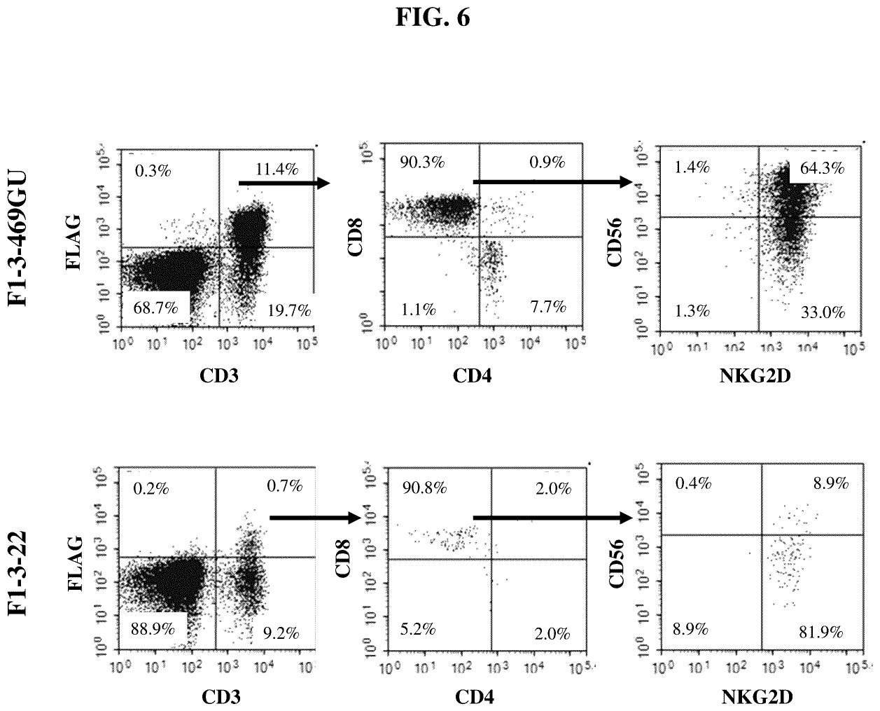

Peripheral blood 28-days post-dosing was examined by flow cytometry for CAR expression and markers of T and NK cells. The results are shown in FIG. 6. A significantly higher number of CAR positive (as detected by FLAG) cells was seen in mice treated subcutaneously with F1-3-469GU modified PBMCs as compared to mice treated intravenously with F1-3-22 modified PBMCs. 11.4% of the cells modified by F1-3-469GU were CAR+CD3+. Within this population, 90.3% of the cells were CD8+CD4-demonstrating a significant skewing toward the CD8 lineage. Further analysis of these CD8+ cells revealed that 64.3% of the cells were CD56+NKG2D+. In contrast, only 0.7% of the cells modified by F1-3-22 were CAR+CD3+. Within this population, 90.8% of the cells were CD8+CD4−. Further analysis of these CD8+ cells revealed that only 8.9% of them were CD56+NKG2D+. These results demonstrate that F1-3-469GU confers a proliferation and/or survival advantage to PBMCs and that the modified CD3+ cells exhibit a T and NK cell-like phenotype in that they are predominantly CD3+, CD8+, and CD56+ as well as NKG2D+.

### Example 6. CAR-TaNKs Produce a Favorable Cytokine Profile with Very Low Levels of CRS-Related Cytokines in a Lymphoreplete Model

In this example, a cell formulation comprising PBMCs genetically modified by a 4-hour incubation with RIPs encoding a CAR and a lymphoproliferative element, were introduced into lymphoreplete hosts and the profile of cytokines present in the blood 21 days after dosing was examined.

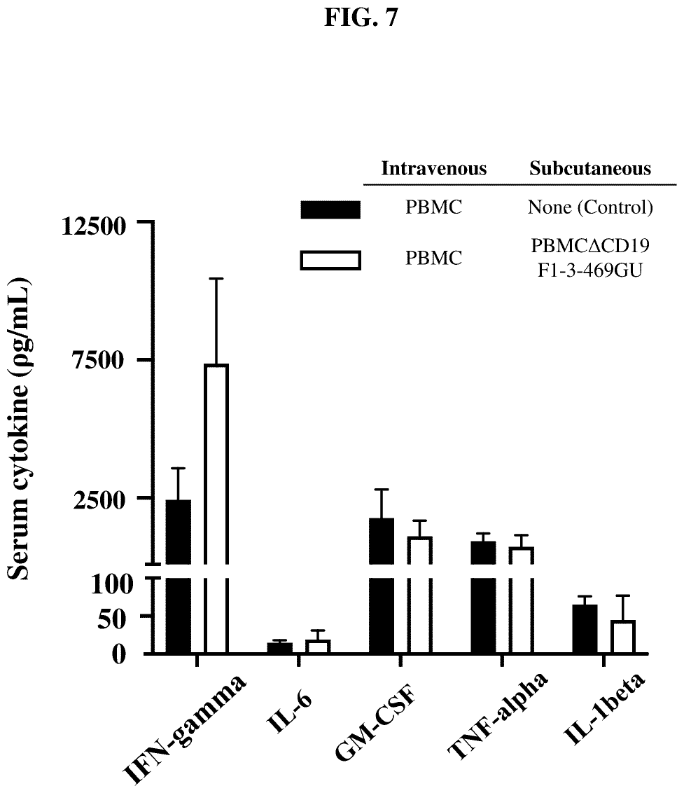

Fresh PBMCs depleted of CD19+ cells were modified with F1-3-469GU using a 4 hour rPOC cell process or mock transduced with PBS as a control. Seven week-old NSG-MHC1/2-DKO mice were reconstituted with human PBMCs on day 0 by injecting 200 μl of donor-matched PBMCs at 5.0×10⁷ cells/mL in PBS-HSA (10 million PBMCs) IV in the tail vein. On day 2, mice were dosed subcutaneously with 6×10⁶ F1-3-469 modified PBMCs or mock transduced PBMCs. 5 mice were included in each group. The profile of cytokines in the blood was assessed 21 days after dosing by Lumix. Results are shown on the graph in FIG. 7. The level of IFNγ in the serum of mice dosed with the CD19 CAR was approximately 7,500 pg/ml which was three times greater than that seen in the control mice. This is not surprising as CAR-T cells are known to secrete large amounts of IFNγ when they encounter antigen or are otherwise activated. Surprisingly, however, no significant increase in IL-6 was seen in the serum of mice dosed with CAR-T cells. Many CAR-T therapies lead to a significant increase in IL-6 which leads to cytokine release syndrome. Similarly, mice dosed with F1-3-469GU modified cells did not show an increase in GM-CSF, TNFα, or IL-6β which are associated with CAR-T related toxicities. Furthermore, there was no increase in the serum levels of any of the other cytokines examined; EGF, EOTAXIN/CCL11, FGF2/FGFB, FLT3L, FRACTALKINE/CX3CL1, GCSF, GM-CSF, GROA, IFNA2, IFNG, ILlA, IL1B, IL1RA, IL2, IL3, IL4, IL5, IL6, L7, IL8/CXCL8, IL9, IL10, IL12P40, IL12P70, IL13, IL15, IL17A/CTLA8, IL17E/IL25, IL17F, IL18, IL22, IL27, IP10/CXCL10, MCP1/CCL2, MCP3/CCL7, MCSF, MDC/CCL22, MIG/CXCL9, MIP1A/CCL3, MIP1B/CCL4, PDGFAA, PDGFAB/BB, RANTES/CCL5, SCD40L, TGFA, TNFα, TNFB/LYMPHOTOXINA(LTA), or VEGF.

This cytokine profile resembles that which would be produced by a population of cells that is comprised largely of NK, NKT, and NKT-like cells.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

TABLE 1
Parts, names, and amino acid sequences for domains of lymphoproliferative parts P1-P2, P1,
P2, P3, and P4.

| Part | Name | Amino Acid Sequence |
|---|---|---|
| M001 | eTAG IL7RA Ins | MLLLVTSLLLCELPHPAFLLIPRKVCNGIGIGEFKDSLSINATNIKHFKNCTSISG |
| | PPCL (interleukin 7 | DLHILPVAFRGDSFTHTPPLDPQELDILKTVKEITGFLLIQAWPENRTDLHAFEN |
| | receptor) | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGPEINNSSGEMDPILLPPCLTISILSFFSVALLVILACVL (SEQ ID |
| | | NO: 84) |
| M002 | eTAG IL7RA Ins | MLLLVTSLLLCELPHPAFLLIPRKVCNGIGIGEFKDSLSINATNIKHFKNCTSISG |
| | PPCL (interleukin 7 | DLHILPVAFRGDSFTHTPPLDPQELDILKTVKEITGFLLIQAWPENRTDLHAFEN |
| | receptor) | LEIIRGRTKQHGQFSLAVVSLNITSLGLRSLKEISDGDVIISGNKNLCYANTINW |
| | | KKLFGTSGQKTKIISNRGENSCKATGQPEINNSSGEMDPILLPPCLTISILSFFSV |
| | | ALLVILACVL (SEQ ID NO: 85) |
| M007 | Myc Tag LMP1 | MEQKLISEEDLEHDLERGPPGPRRPPRGPPLSSSLGLALLLLLLALLFWLYIVM |
| | NC_007605_1 | SDWTGGALLVLYSFALMLIIIILIIFIFRRDLLCPLGALCILLLMITLLLIALWNLH |
| | | GQALFLGIVLFIFGCLLVLGIWIYLLEMLWRLGATIWQLLAFFLAFFLDLILLIIA |
| | | LYLQQNWWTLLVDLLWLLLFLAILIWM (SEQ ID NO: 86) |
| M008 | Myc LMP1 | MEQKLISEEDLSSSLGLALLLLLLALLFWLYIVMSDWTGGALLVLYSFALMLII |
| | NC_007605_1 | IILIIFIFRRDLLCPLGALCILLLMITLLLIALWNLHGQALFLGIVLFIFGCLLVLGI |
| | | WIYLLEMLWRLGATIWQLLAFFLAFFLDLILLIIALYLQQNWWTLLVDLLWLL |
| | | LFLAILIWM (SEQ ID NO: 87) |
| M009 | LMP1 NC_007605_1 | MEHDLERGPPGPRRPPRGPPLSSSLGLALLLLLLALLFWLYIVMSDWTGGALL |
| | | VLYSFALMLIIIILIIFIFRRDLLCPLGALCILLLMITLLLIALWNLHGQALFLGIVL |
| | | FIFGCLLVLGIWIYLLEMLWRLGATIWQLLAFFLAFFLDLILLIIALYLQQNWW |
| | | TLLVDLLWLLLFLAILIWM (SEQ ID NO: 88) |
| M010 | LMP1 NC_007605 1 | MSLGLALLLLLLALLFWLYIVMSDWTGGALLVLYSFALMLIIIILIIFIFRRDLLC |
| | | PLGALCILLLMITLLLIALWNLHGQALFLGIVLFIFGCLLVLGIWIYLLEMLWRL |
| | | GATIWQLLAFFLAFFLDLILLIIALYLQQNWWTLLVDLLWLLLFLAILIWM |
| | | (SEQ ID NO: 89) |
| M012 | eTAG CRLF2 | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_022148_3 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGAETPTPPKPKLSKCILISSLAILLMVSLLLLSLW (SEQ ID NO: 90) |
| M013 | eTAG CRLF2 | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_022148_3 | LEIIRGRTKQHGQFSLAVVSLNITSLGLRSLKEISDGDVIISGNKNLCYANTINW |
| | | KKLFGTSGQKTKIISNRGENSCKATGQAETPTPPKPKLSKCILISSLAILLMVSL |
| | | LLLSLW (SEQ ID NO: 91) |
| M018 | eTAG CSF2RB | MLLLVTSLLLCELPHPAFLLIPRKVCNGIGIGEFKDSLSINATNIKHFKNCTSISG |
| | NM_000395_2 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGTESVLPMWVLALIEIFLTIAVLLAL (SEQ ID NO: 92) |
| M019 | eTAG CSF2RB | MLLLVTSLLLCELPHPAFLLIPRKVCNGIGIGEFKDSLSINATNIKHFKNCTSISG |
| | NM_000395_2 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQTESVLPMWVLALIEIFLTIAVLLAL |
| | | (SEQ ID NO: 93) |
| M024 | eTAG CSF3R | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000760_3 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGTPEGSELHIILGLFGLLLLLNCLCGTAWLCC (SEQ ID NO: 94) |
| M025 | eTAG CSF3R | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000760_3 | LEIIRGRTKQHGQFSLAVVSLNITSLGLRSLKEISDGDVIISGNKNLCYANTINW |
| | | KKLFGTSGQKTKIISNRGENSCKATGQTPEGSELHIILGLFGLLLLLNCLCGTA |
| | | WLCC (SEQ ID NO: 95) |
| M030 | eTAG EPOR | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000121_3 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGTPSDLDPCCLTLSLILVVILVLLTVLALLS (SEQ ID NO: 96) |
| M031 | eTAG EPOR | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000121_3 | LEIIRGRTKQHGQFSLAVVSLNITSLGLRSLKEISDGDVIISGNKNLCYANTINW |
| | | KKLFGTSGQKTKIISNRGENSCKATGQTPSDLDPCCLTLSLILVVILVLLTVLAL |
| | | LS (SEQ ID NO: 97) |
| M036 | eTAG GHR | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000163_4 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGTLPQMSQFTCCEDFYFPWLLCIIFGIFGLTVMLFVFLFS (SEQ ID |
| | | NO: 98) |
| M037 | eTAG GHR | MLLLVTSLLLCELPHPAFLLIPRKVCNGIGIGEFKDSLSINATNIKHFKNCTSISG |
| | transcript variant 1 | DLHILPVAFRGDSFTHTPPLDPQELDILKTVKEITGFLLIQAWPENRTDLHAFEN |
| | NM_000163_4 | LEIIRGRTKQHGQFSLAVVSLNITSLGLRSLKEISDGDVIISGNKNLCYANTINW |
| | | KKLFGTSGQKTKIISNRGENSCKATGQTLPQMSQFTCCEDFYFPWLLCIIFGIFG |
| | | LTVMLFVFLFS (SEQ ID NO: 99) |
| M042 | eTAG truncated after | MLLLVTSLLLCELPHPAFLLIPRKVCNGIGIGEFKDSLSINATNIKHFKNCTSISG |
| | Fn F523C IL27RA | DLHILPVAFRGDSFTHTPPLDPQELDILKTVKEITGFLLIQAWPENRTDLHAFEN |
| | NM_004843_3 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGHLPDNTLRWKVLPGILCLWGLFLLGCGLSLA (SEQ ID NO: 100) |
| M043 | eTAG truncated after | MLLLVTSLLLCELPHPAFLLIPRKVCNGIGIGEFKDSLSINATNIKHFKNCTSISG |
| | Fn F523C IL27RA | DLHILPVAFRGDSFTHTPPLDPQELDILKTVKEITGFLLIQAWPENRTDLHAFEN |
| | NM_004843_3 | LEIIRGRTKQHGQFSLAVVSLNITSLGLRSLKEISDGDVIISGNKNLCYANTINW |
| | | KKLFGTSGQKTKIISNRGENSCKATGQHLPDNTLRWKVLPGILCLWGLFLLGC |
| | | GLSLA (SEQ ID NO: 101) |
| M048 | eTAG truncated after | MLLLVTSLLLCELPHPAFLLIPRKVCNGIGIGEFKDSLSINATNIKHFKNCTSISG |
| | Fn S505N MPL | DLHILPVAFRGDSFTHTPPLDPQELDILKTVKEITGFLLIQAWPENRTDLHAFEN |
| | NM_005373_2 | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGETATETAWISLVTALHLVLGLNAVLGLLLL (SEQ ID NO: 102) |
| M049 | eTAG truncated after | MLLLVTSLLLCELPHPAFLLIPRKVCNGIGIGEFKDSLSINATNIKHFKNCTSISG |
| | Fn S505N MPL | DLHILPVAFRGDSFTHTPPLDPQELDILKTVKEITGFLLIQAWPENRTDLHAFEN |
| | NM_005373_2 | LEIIRGRTKQHGQFSLAVVSLNITSLGLRSLKEISDGDVIISGNKNLCYANTINW |
| | | KKLFGTSGQKTKIISNRGENSCKATGQETATETAWISLVTALHLVLGLNAVLG |
| | | LLLL (SEQ ID NO: 103) |
| E006 | eTag 0A JUN | MLLLVTSLLLCELPHPAFLLIPRKVCNGIGIGEFKDSLSINATNIKHFKNCTSISG |
| | NM_002228_3 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKV (SEQ ID |
| | | NO: 104) |
| E007 | eTag 1A JUN | MLLLVTSLLLCELPHPAFLLIPRKVCNGIGIGEFKDSLSINATNIKHFKNCTSISG |
| | NM_002228_3 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVA (SEQ |
| | | ID NO: 105) |
| E008 | eTag 2A JUN | MLLLVTSLLLCELPHPAFLLIPRKVCNGIGIGEFKDSLSINATNIKHFKNCTSISG |
| | NM_002228_3 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKFGTSGQKTKIISNRGENSCKATGQVCHALCSPEGCWGPEPRDCVSCRNVSR |
| | | GRECVDKCNLLEGEPREFVENSECIQCHPECLPQAMNITCTGRGPDNCIQCAH |
| | | YIDGPHCVKTCPAGVMGENNTLVWKYADAGHVCHLCHPNCTYGCTGPGLE |
| | | GCPTNGLERIARLEEKVKTLKAQNSELASTANMLREQVAQLKQKVAA (SEQ |
| | | ID NO: 106) |
| E009 | eTag 3A JUN | MLLLVTSLLLCELPHPAFLLIPRKVCNGIGIGEFKDSLSINATNIKHFKNCTSISG |
| | NM_002228_3 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVAAA |
| | | (SEQ ID NO: 107) |
| E010 | eTag 4A JUN | MLLLVTSLLLCELPHPAFLLIPRKVCNGIGIGEFKDSLSINATNIKHFKNCTSISG |
| | NM_002228_3 | DLHILPVAFRGDSFTHTPPLDPQELDILKTVKEITGFLLIQAWPENRTDLHAFEN |
| | | LEIIRGRTKQHGQFSLAVVSLNITSLGLRSLKEISDGDVIISGNKNLCYANTINW |
| | | KKLFGTSGQKTKIISNRGENSCKATGQVCHALCSPEGCWGPEPRDCVSCRNVS |
| | | RGRECVDKCNLLEGEPREFVENSECIQCHPECLPQAMNITCTGRGPDNCIQCA |
| | | HYIDGPHCVKTCPAGVMGENNTLVWKYADAGHVCHLCHPNCTYGCTGPGL |
| | | EGCPTNGLERIARLEEKVKTLKAQNSELASTANMLREQVAQLKQKVAAAA |
| | | (SEQ ID NO: 108) |
| E011 | Myc Tag 0A JUN | MTILGTTFGMVFSLLQVVSGEQKLISEEDLLERIARLEEKVKTLKAQNSELAST |
| | NM_002228_3 | ANMLREQVAQLKQKV (SEQ ID NO: 109) |
| E012 | Myc Tag 1A JUN | MTILGTTFGMVFSLLQVVSGEQKLISEEDLLERIARLEEKVKTLKAQNSELAST |
| | NM_002228_3 | ANMLREQVAQLKQKVA (SEQ ID NO: 110) |
| E013 | Myc Tag 2A JUN | MTILGTTFGMVFSLLQVVSGEQKLISEEDLLERIARLEEKVKTLKAQNSELAST |
| | NM_002228_3 | ANMLREQVAQLKQKVAA (SEQ ID NO: 111) |
| E014 | Myc Tag 3A JUN | MTILGTTFGMVFSLLQVVSGEQKLISEEDLLERIARLEEKVKTLKAQNSELAST |
| | NM_002228_3 | ANMLREQVAQLKQKVAAA (SEQ ID NO: 112) |
| E015 | Myc Tag 4A JUN | MTILGTTFGMVFSLLQVVSGEQKLISEEDLLERIARLEEKVKTLKAQNSELAST |
| | NM_002228_3 | ANMLREQVAQLKQKVAAAA (SEQ ID NO: 113) |
| E016 | 0A JUN | LERIARLEEKVKTLKAQNSELASTANMLREQVAQLKQKV |
| | NM_002228_3 | |
| T001 | CD2 transcript | LIIGICGGGSLLMVFVALLVFYI (SEQ ID NO: 114) |
| | variant 1 | |
| | NM_001328609_1 | |
| T002 | CD3D transcript | GIIVTDVIATLLLALGVFCFA (SEQ ID NO: 115) |
| | variant 1 | |
| | NM_000732_4 | |
| T003 | CD3E | VMSVATIVIVDICITGGLLLLVYYWS (SEQ ID NO: 116) |
| | NM_000733_3 | |
| T004 | CD3G | GFLFAEIVSIFVLAVGVYFIA (SEQ ID NO: 117) |
| | NM_000073_2 | |
| T005 | CD3Z CD247 | LCYLLDGILFIYGVILTALFL (SEQ ID NO: 118) |
| | transcript variant 1 | |
| | NM_198053_2 | |
| T006 | CD4 transcript | MALIVLGGVAGLLLFIGFIGLGIFF (SEQ ID NO: 119) |
| | variant 1 and 2 | |
| | NM_000616_4 | |
| T007 | CD8A transcript | IYIWAPLAGTCGVLLLSLVIT (SEQ ID NO: 120) |
| | variant 1 | |
| | NM_001768_6 | |
| T008 | CD8B transcript | LGLLVAGVLVLLVSLGVAIHLCC (SEQ ID NO: 121) |
| | variant 2 | |
| | NM_172213_3 | |
| T009 | CD27 NM_001242_4 | ILVIFSGMFLVFTLAGALFLH (SEQ ID NO: 122) |
| T010 | CD28 transcript | FWVLVVVGGVLACYSLLVTVAFIIFWV (SEQ ID NO: 123) |
| | variant 1 | |
| | NM_006139_3 | |
| T011 | CD40 transcript | ALVVIPIIFGILFAILLVLVFI (SEQ ID NO: 124) |
| | variant 1 and 6 | |
| | NM_001250_5 | |
| T012 | CD79A transcript | IITAEGIILLFCAVVPGTLLLF (SEQ ID NO: 125) |
| | variant 1 | |
| | NM_001783_3 | |
| T013 | CD79B transcript | GIIMIQTLLIILFIIVPIFLLL (SEQ ID NO: 126) |
| | variant 3 | |
| | NM_001039933_2 | |
| T014 | CRLF2 transcript | FILISSLAILLMVSLLLLSLW (SEQ ID NO: 127) |
| | variant 1 | |
| | NM_022148_3 | |
| T015 | CRLF2 transcript | CILISSLAILLMVSLLLLSLW (SEQ ID NO: 128) |
| | variant 1 | |
| | NM_022148_3 | |
| T016 | CSF2RA transcript | NLGSVYIYVLLIVGTLVCGIVLGFLF (SEQ ID NO: 129) |
| | variant 7 and 8 | |
| | NM_001161529_1 | |
| T017 | CSF2RB | MWVLALIVIFLTIAVLLAL (SEQ ID NO: 130) |
| | NM_000395_2 | |
| T018 | CSF2RB | MWVLALIEIFLTIAVLLAL (SEQ ID NO: 131) |
| | NM_000395_2 | |
| T019 | CSF3R transcript | IILGLFGLLLLLTCLCGTAWLCC (SEQ ID NO: 132) |
| | variant 1 | |
| | NM_000760_3 | |
| T020 | CSF3R transcript | IILGLFGLLLLLNCLCGTAWLCC (SEQ ID NO: 133) |
| | variant 1 | |
| | NM_000760_3 | |
| T021 | EPOR transcript | LILTLSLILVVILVLLTVLALLS (SEQ ID NO: 134) |
| | variant 1 | |
| | NM_000121_3 | |
| T022 | EPOR transcript | CCLTLSLILVVILVLLTVLALLS (SEQ ID NO: 135) |
| | variant 1 | |
| | NM_000121_3 | |
| T023 | FCER1G | LCYILDAILFLYGIVLTLLYC (SEQ ID NO: 136) |
| | NM_004106_1 | |
| T024 | FCGR2C | IIVAVVTGIAVAAIVAAVVALIY (SEQ ID NO: 137) |
| | NM_201563_5 | |
| T025 | FCGRA2 transcript | IIVAVVIATAVAAIVAAVVALIY (SEQ ID NO: 138) |
| | variant 1 | |
| | NM_001136219_1 | |
| T026 | GHR transcript | FPWLLIIIFGIFGLTVMLFVFLFS (SEQ ID NO: 139) |
| | variant 1 | |
| | NM_000163_4 | |
| T027 | GHR transcript | FPWLLCIIFGIFGLTVMLFVFLFS (SEQ ID NO: 140) |
| | variant 1 | |
| | NM_000163_4 | |
| T028 | ICOS NM_012092.3 | FWLPIGCAAFVVVCILGCILI (SEQ ID NO: 141) |
| T029 | IFNAR1 | IWLIVGICIALFALPFVIYAA (SEQ ID NO: 142) |
| | NM_000629_2 | |
| T030 | IFNAR2 transcript | IGGIITVFLIALVLTSTIVTL (SEQ ID NO: 143) |
| | variant 1 | |
| | NM_207585_2 | |
| T031 | IFNGR1 | SLWIPVVAALLLFLVLSLVFI (SEQ ID NO: 144) |
| | NM_000416_2 | |
| T032 | IFNGR2 transcript | VILISVGTFSLLSVLAGACFF (SEQ ID NO: 145) |
| | variant 1 | |
| | NM_001329128_1 | |
| T033 | IFNLR1 | FLVLPSLLILLLVIAAGGVIW (SEQ ID NO: 146) |
| | NM_170743_3 | |
| T034 | IL1R1 transcript | HMIGICVTLTVIIVCSVFIYKIF (SEQ ID NO: 147) |
| | variant 2 | |
| | NM_001288706_1 | |
| T035 | IL1RAP transcript | VLLVVILIVVYHVYWLEMVLF (SEQ ID NO: 148) |
| | variant 1 | |
| | NM_002182_3 | |
| T036 | IL1RL1 transcript | IYCIIAVCSVFLMLINVLVII (SEQ ID NO: 149) |
| | variant 1 | |
| | NM_016232.4 | |
| T037 | IL1RL2 | AYLIGGLIALVAVAVSVVYIY (SEQ ID NO: 150) |
| | NM_003854.2 | |
| T038 | IL2RA transcript | VAVAGCVFLLISVLLLSGL (SEQ ID NO: 151) |
| | variant 1 | |
| | NM_000417_2 | |
| T039 | IL2RB transcript | IPWLGHLLVGLSGAFGFIILVYLLI (SEQ ID NO: 152) |
| | variant 1 | |
| | NM_000878_4 | |
| T040 | IL2RG | VVISVGSMGLIISLLCVYFWL (SEQ ID NO: 153) |
| | NM_000206_2 | |
| T041 | IL3RA transcript | TSLLIALGTLLALVCVFVIC (SEQ ID NO: 154) |
| | variant 1 and 2 | |
| | NM_002183_3 | |
| T042 | IL4R transcript | LLLGVSVSCIVILAVCLLCYVSIT (SEQ ID NO: 155) |
| | variant 1 | |
| | NM_000418_3 | |
| T043 | IL5RA transcript | FVIVIMATICFILLILSLIC (SEQ ID NO: 156) |
| | variant 1 | |
| | NM_000564_4 | |
| T044 | IL6R transcript | TFLVAGGSLAFGTLLCIAIVL (SEQ ID NO: 157) |
| | variant 1 | |
| | NM_000565_3 | |
| T045 | IL6ST transcript | AIVVPVCLAFLLTTLLGVLFCF (SEQ ID NO: 158) |
| | variant 1 and 3 | |
| | NM_002184_3 | |
| T046 | IL7RA | ILLTISILSFFSVALLVILACVL (SEQ ID NO: 159) |
| | NM_002185_3 | |
| T047 | IL7RA Ins PPCL | ILLPPCLTISILSFFSVALLVILACVL (SEQ ID NO: 160) |
| | (interleukin 7 | |
| | receptor) | |
| T048 | IL9R transcript | GNTLVAVSIFLLLTGPTYLLF (SEQ ID NO: 161) |
| | variant 1 | |
| | NM_002186_2 | |
| T049 | IL10RA transcript | VIIFFAFVLLLSGALAYCLAL (SEQ ID NO: 162) |
| | variant 1 | |
| | NM_001558_3 | |
| T050 | IL10RB | WMVAVILMASVFMVCLALLGCF (SEQ ID NO: 163) |
| | NM_000628_4 | |
| T051 | IL11RA | SLGILSFLGLVAGALALGLWL (SEQ ID NO: 164) |
| | NM_001142784_2 | |
| T052 | IL12RB1 transcript | WLIFFASLGSFLSILLVGVLGYLGL (SEQ ID NO: 165) |
| | variant 1 and 4 | |
| | NM_005535_2 | |
| T053 | IL12RB2 transcript | WMAFVAPSICIAIIMVGIFST (SEQ ID NO: 166) |
| | variant 1 and 3 | |
| | NM_001559_2 | |
| T054 | IL13RA1 | LYITMLLIVPVIVAGAIIVLLLYL (SEQ ID NO: 167) |
| | NM_001560_2 | |
| T055 | IL13RA2 | FWLPFGFILILVIFVTGLLL (SEQ ID NO: 168) |
| | NM_000640_2 | |
| T056 | IL15RA transcript | VAISTSTVLLCGLSAVSLLACYL (SEQ ID NO: 169) |
| | variant 4 | |
| | NM_001256765_1 | |
| T057 | IL17RA | VYWFITGISILLVGSVILLIV (SEQ ID NO: 170) |
| | NM_014339_6 | |
| T058 | IL17RB | LLLLSLLVATWVLVAGIYLMW (SEQ ID NO: 171) |
| | NM_018725_3 | |
| T059 | IL17RC transcript | WALVWLACLLFAAALSLILLL (SEQ ID NO: 172) |
| | variant 1 | |
| | NM_153460_3 | |
| T060 | IL17RD transcript | AVAITVPLVVISAFATLFTVM (SEQ ID NO: 173) |
| | variant 2 | |
| | NM_017563_4 | |
| T061 | IL17RE transcript | LGLLILALLALLTLLGVVLAL (SEQ ID NO: 174) |
| | variant 1 | |
| | NM_153480_1 | |
| T062 | IL18R1 transcript | GMIIAVLILVAVVCLVTVCVI (SEQ ID NO: 175) |
| | variant 1 | |
| | NM_003855_3 | |
| T063 | IL18RAP | GVVLLYILLGTIGTLVAVLAA (SEQ ID NO: 176) |
| | NM_003853_3 | |
| T064 | IL20RA transcript | IIFWYVLPISITVFLFSVMGY (SEQ ID NO: 177) |
| | variant 1 | |
| | NM_014432_3 | |
| T065 | IL20RB | VLALFAFVGFMLILVVVPLFV (SEQ ID NO: 178) |
| | NM_144717_3 | |
| T066 | IL21R transcript | GWNPHLLLLLLLVIVFIPAFW (SEQ ID NO: 179) |
| | variant 2 | |
| | NM_181078_2 | |
| T067 | IL22RA1 | YSFSGAFLFSMGFLVAVLCYL (SEQ ID NO: 180) |
| | NM_021258_3 | |
| T068 | IL23R | LLLGMIVFAVMLSILSLIGIF (SEQ ID NO: 181) |
| | NM_144701_2 | |
| T069 | IL27RA | VLPGILFLWGLFLLGCGLSLA (SEQ ID NO: 182) |
| | NM_004843_3 | |
| T070 | IL27RA | VLPGILCLWGLFLLGCGLSLA (SEQ ID NO: 183) |
| | NM_004843_3 | |
| T071 | IL31RA transcript | IILITSLIGGGLLILIILTVAYGL (SEQ ID NO: 184) |
| | variant 1 | |
| | NM_139017_5 | |
| T072 | LEPR transcript | AGLYVIVPVIISSSILLLGTLLI (SEQ ID NO: 185) |
| | variant 1 | |
| | NM_002303_5 | |
| T073 | LIFR | VGLIIAILIPVAVAVIVGVVTSILC (SEQ ID NO: 186) |
| | NM_001127671_1 | |
| T074 | MPL NM_005373_2 | ISLVTALHLVLGLSAVLGLLLL (SEQ ID NO: 187) |
| T075 | MPL NM_005373_2 | ISLVTALHLVLGLNAVLGLLLL (SEQ ID NO: 188) |
| T076 | OSMR transcript | LIHILLPMVFCVLLIMVMCYL (SEQ ID NO: 189) |
| | variant 4 | |
| | NM_001323505_1 | |
| T077 | PRLR transcript | TTVWISVAVLSAVICLIIVWAVAL (SEQ ID NO: 190) |
| | variant 1 | |
| | NM_000949_6 | |
| T078 | TNFRSF4 | VAAILGLGLVLGLLGPLAILL (SEQ ID NO: 191) |
| | NM_003327_3 | |
| T079 | TNFRSF8 transcript | PVLDAGPVLFWVILVLVVVVGSSAFLLC (SEQ ID NO: 192) |
| | variant 1 | |
| | NM_001243_4 | |
| T080 | TNFRSF9 | IISFFLALTSTALLFLLFFLTLRFSVV (SEQ ID NO: 193) |
| | NM_001561_5 | |
| T081 | TNFRSF14 transcript | WWFLSGSLVIVIVCSTVGLII (SEQ ID NO: 194) |
| | variant 1 | |
| | NM_003820_3 | |
| T082 | TNFRSF18 transcript | LGWLTVVLLAVAACVLLLTSA (SEQ ID NO: 195) |
| | variant 1 | |
| | NM_004195_2 | |
| S036 | CD2 transcript | TKRKKQRSRRNDEELETRAHRVATEERGRKPHQIPASTPQNPATSQHPPPPPG |
| | variant 1 | HRSQAPSHRPPPPGHRVQHQPQKRPPAPSGTQVHQQKGPPLPRPRVQPKPPHG |
| | NM_001328609_1 | AAENSLSPSSN (SEQ ID NO: 196) |
| S037 | CD3D transcript | GHETGRLSGAADTQALLRNDQVYQPLRDRDDAQYSHLGGNWARNK (SEQ |
| | variant 1 | ID NO: 197) |
| | NM_000732_4 | |
| S038 | CD3E | KNRKAKAKPVTRGAGAGGRQRGQNKERPPPVPNPDYEPIRKGQRDLYSGLN |
| | NM_000733_3 | QRRI (SEQ ID NO: 198) |
| S039 | CD3G | GQDGVRQSRASDKQTLLPNDQLYQPLKDREDDQYSHLQGNQLRRN (SEQ ID |
| | NM_000073_2 | NO: 199) |
| S042 | CD4 transcript | CVRCRHRRRQAERMSQIKRLLSEKKTCQCPHRFQKTCSPI (SEQ ID NO: 200) |
| | variant 1 and 2 | |
| | NM_000616_4 | |
| S043 | CD8A transcript | LYCNHRNRRRVCKCPRPVVKSGDKPSLSARYV (SEQ ID NO: 201) |
| | variant 1 | |
| | NM_001768_6 | |
| S044 | CD8B transcript | RRRRARLRFMKQPQGEGISGTFVPQCLHGYYSNTTTSQKLLNPWILKT (SEQ |
| | variant 2 | ID NO: 202) |
| | NM_172213_3 | |
| S045 | CD8B transcript | RRRRARLRFMKQLRLHPLEKCSRMDY (SEQ ID NO: 203) |
| | variant 3 | |
| | NM_172101_3 | |
| S046 | CD8B transcript | RRRRARLRFMKQFYK (SEQ ID NO: 204) |
| | variant 5 | |
| | NM_004931_4 | |
| S047 | CD27 NM_001242_4 | QRRKYRSNKGESPVEPAEPCRYSCPREEEGSTIPIQEDYRKPEPACSP (SEQ ID |
| | | NO: 205) |
| S048 | mutated Delta Lck | RSKRSRLLHSDYMNMTPRRPGPTRKHYQAYAAARDFAAYRS (SEQ ID |
| | CD28 transcript | NO: 206) |
| | variant 1 | |
| | NM_006139_3 | |
| S049 | CD28 transcript | RSKRSRLLHSDYMNMTPRRPGPTRKHYQPYAPPRDFAAYRS (SEQ ID |
| | variant 1 | NO: 207) |
| | NM_006139_3 | |
| S050 | CD40 transcript | KKVAKKPTNKAPHPKQEPQEINFPDDLPGSNTAAPVQETLHGCQPVTQEDGK |
| | variant 1 and 6 | ESRISVQERQ (SEQ ID NO: 208) |
| | NM_001250_5 | |
| S051 | CD40 transcript | SESSEKVAKKPTNKAPHPKQEPQEINFPDDLPGSNTAAPVQETLHGCQPVTQE |
| | variant 5 | DGKESRISVQERQ (SEQ ID NO: 209) |
| | NM_001322421_1 | |
| S052 | CD79A transcript | RKRWQNEKLGLDAGDEYEDENLYEGLNLDDCSMYEDISRGLQGTYQDVGSL |
| | variant 1 | NIGDVQLEKP (SEQ ID NO: 210) |
| | NM_001783_3 | |
| S053 | CD79B transcript | LDKDDSKAGMEEDHTYEGLDIDQTATYEDIVTLRTGEVKWSVGEHPGQE |
| | variant 3 | (SEQ ID NO: 211) |
| | NM_001039933_2 | |
| S054 | CRLF2 transcript | KLWRVKKFLIPSVPDPKSIFPGLFEIHQGNFQEWITDTQNVAHLHKMAGAEQE |
| | variant 1 | SGPEEPLVVQLAKTEAESPRMLDPQTEEKEASGGSLQLPHQPLQGGDVVTIGG |
| | NM_022148_3 | FTFVMNDRSYVAL (SEQ ID NO: 212) |
| S057 | CSF2RB | RFCGIYGYRLRRKWEEKIPNPSKSHLFQNGSAELWPPGSMSAFTSGSPPHQGP |
| | NM_000395_2 | WGSRFPELEGVFPVGFGDSEVSPLTIEDPKHVCDPPSGPDTTPAASDLPTEQPPS |
| | | PQPGPPAASHTPEKQASSFDFNGPYLGPPHSRSLPDILGQPEPPQEGGSQKSPPP |
| | | GSLEYLCLPAGGQVQLVPLAQAMGPGQAVEVERRPSQGAAGSPSLESGGGPA |
| | | PPALGPRVGGQDQKDSPVAIPMSSGDTEDPGVASGYVSSADLVFTPNSGASSV |
| | | SLVPSLGLPSDQTPSLCPGLASGPPGAPGPVKSGFEGYVELPPIEGRSPRSPRNN |
| | | PVPPEAKSPVLNPGERPADVSPTSPQPEGLLVLQQVGDYCFLPGLGPGPLSLRS |
| | | KPSSPGPGPEIKNLDQAFQVKKPPGQAVPQVPVIQLFKALKQQDYLSLPPWEV |
| | | NKPGEVC (SEQ ID NO: 213) |
| S058 | CSF2RA transcript | KRFLRIQRLFPPVPQIKDKLNDNHEVEDEIIWEEFTPEEGKGYREEVLTVKEIT |
| | variant 7 and 8 | (SEQ ID NO: 214) |
| | NM_001161529_1 | |
| S059 | CSF2RA transcript | KRFLRIQRLFPPVPQIKDKLNDNHEVEDEMGPQRHHRCGWNLYPTPGPSPGSG |
| | variant 9 | SSPRLGSESSL (SEQ ID NO: 215) |
| | NM_001161531_1 | |
| S062 | CSF3R transcript | SPNRKNPLWPSVPDPAHSSLGSWVPTIMEEDAFQLPGLGTPPITKLTVLEEDEK |
| | variant 1 | KPVPWESHNSSETCGLPTLVQTYVLQGDPRAVSTQPQSQSGTSDQVLYGQLL |
| | NM_000760_3 | GSPTSPGPGHYLRCDSTQPLLAGLTPSPKSYENLWFQASPLGTLVTPAPSQEDD |
| | | CVFGPLLNFPLLQGIRVHGMEALGSF (SEQ ID NO: 216) |
| S063 | CSF3R transcript | SPNRKNPLWPSVPDPAHSSLGSWVPTIMEELPGPRQGQWLGQTSEMSRALTP |
| | variant 3 | HPCVQDAFQLPGLGTPPITKLTVLEEDEKKPVPWESHNSSETCGLPTLVQTYV |
| | NM_156039_3 | LQGDPRAVSTQPQSQSGTSDQVLYGQLLGSPTSPGPGHYLRCDSTQPLLAGLT |
| | | PSPKSYENLWFQASPLGTLVTPAPSQEDDCVFGPLLNFPLLQGIRVHGMEALG |
| | | SF (SEQ ID NO: 217) |
| S064 | CSF3R transcript | SPNRKNPLWPSVPDPAHSSLGSWVPTIMEEDAFQLPGLGTPPITKLTVLEEDEK |
| | variant 4 | KPVPWESHNSSETCGLPTLVQTYVLQGDPRAVSTQPQSQSGTSDQAGPPRRSA |
| | NM_172313_2 | YFKDQIMLHPAPPNGLLCLFPITSVL (SEQ ID NO: 218) |
| S069 | EPOR transcript | HRRALKQKIWPGIPSPESEFEGLFTTHKGNFQLWLYQNDGCLWWSPCTPFTED |
| | variant 1 | PPASLEVLSERCWGTMQAVEPGTDDEGPLLEPVGSEHAQDTYLVLDKWLLPR |
| | NM_000121_3 | NPPSEDLPGPGGSVDIVAMDEGSEASSCSSALASKPSPEGASAASFEYTILDPSS |
| | | QLLRPWTLCPELPPTPPHLKYLYLVVSDSGISTDYSSGDSQGAQGGLSDGPYS |
| | | NPYENSLIPAAEPLPPSYVACS (SEQ ID NO: 219) |
| S072 | EPOR transcript | HRRALKQKIWPGIPSPESEFEGLFTTHKGNFQLWLYQNDGCLWWSPCTPFTED |
| | variant 1 | PPASLEVLSERCWGTMQAVEPGTDDEGPLLEPVGSEHAQDTYLVLDKWLLPR |
| | NM_000121_3 | NPPSEDLPGPGGSVDIVAMDEGSEASSCSSALASKPSPEGASAASFEYTILDPSS |
| | | QLLRPWTLCPELPPTPPHLKFLFLVVSDSGISTDYSSGDSQGAQGGLSDGPYSN |
| | | PYENSLIPAAEPLPPSYVACS (SEQ ID NO: 220) |
| S074 | FCER1G | RLKIQVRKAAITSYEKSDGVYTGLSTRNQETYETLKHEKPPQ (SEQ ID |
| | NM_004106_1 | NO: 221) |
| S075 | FCGR2C | CRKKRISANSTDPVKAAQFEPPGRQMIAIRKRQPEETNNDYETADGGYMTLNP |
| | NM_201563_5 | RAPTDDDKNIYLTLPPNDHVNSNN (SEQ ID NO: 222) |
| S076 | FCGRA2 transcript | CRKKRISANSTDPVKAAQFEPPGRQMIAIRKRQLEETNNDYETADGGYMTLN |
| | variant 1 | PRAPTDDDKNIYLTLPPNDHVNSNN (SEQ ID NO: 223) |
| | NM_001136219_1 | |
| S077 | GHR transcript | KQQRIKMLILPPVPVPKIKGIDPDLLKEGKLEEVNTILAIHDSYKPEFHSDDSW |
| | variant 1 | VEFIELDIDEPDEKTEESDTDRLLSSDHEKSHSNLGVKDGDSGRTSCCEPDILET |
| | NM_000163_4 | DFNANDIHEGTSEVAQPQRLKGEADLLCLDQKNQNNSPYHDACPATQQPSVI |
| | | QAEKNKPQPLPTEGAESTHQAAHIQLSNPSSLSNIDFYAQVSDITPAGSVVLSP |
| | | GQKNKAGMSQCDMHPEMVSLCQENFLMDNAYFCEADAKKCIPVAPHIKVES |
| | | HIQPSLNQEDIYITTESLTTAAGRPGTGEHVPGSEMPVPDYTSIHIVQSPQGLIL |
| | | NATALPLPDKEFLSSCGYVSTDQLNKIMP (SEQ ID NO: 224) |
| S080 | ICOS NM_012092.3 | CWLTKKKYSSSVHDPNGEYMFMRAVNTAKKSRLTDVTL (SEQ ID NO: 225) |
| S081 | IFNAR1 | KVFLRCINYVFFPSLKPSSSIDEYFSEQPLKNLLLSTSEEQIEKCFIIENISTIATVE |
| | NM_000629_2 | ETNQTDEDHKKYSSQTSQDSGNYSNEDESESKTSEELQQDFV (SEQ ID |
| | | NO: 226) |
| S082 | IFNAR2 transcript | KWIGYICLRNSLPKVLNFHNFLAWPFPNLPPLEAMDMVEVIYINRKKKVWDY |
| | variant 1 | NYDDESDSDTEAAPRTSGGGYTMHGLTVRPLGQASATSTESQLIDPESEEEPD |
| | NM_207585_2 | LPEVDVELPTMPKDSPQQLELLSGPCERRKSPLQDPFPEEDYSSTEGSGGRITF |
| | | NVDLNSVFLRVLDDEDSDDLEAPLMLSSHLEEMVDPEDPDNVQSNHLLASGE |
| | | GTQPTFPSPSSEGLWSEDAPSDQSDTSESDVDLGDGYIMR (SEQ ID NO: 227) |
| S083 | IFNAR2 transcript | KWIGYICLRNSLPKVLRQGLAKGWNAVAIHRCSHNALQSETPELKQSSCLSFP |
| | variant 2 | SSWDYKRASLCPSD (SEQ ID NO: 228) |
| | NM_000874_4 | |
| S084 | IFNGR1 | CFYIKKINPLKEKSIILPKSLISVVRSATLETKPESKYVSLITSYQPFSLEKEVVCE |
| | NM_000416_2 | EPLSPATVPGMHTEDNPGKVEHTEELSSITEVVTTEENIPDVVPGSHLTPIERES |
| | | SSPLSSNQSEPGSIALNSYHSRNCSESDHSRNGFDTDSSCLESHSSLSDSEFPPN |
| | | NKGEIKTEGQELITVIKAPTSFGYDKPHVLVDLLVDDSGKESLIGYRPTEDSKE |
| | | FS (SEQ ID NO: 229) |
| S085 | IFNGR2 transcript | LVLKYRGLIKYWFHTPPSIPLQIEEYLKDPTQPILEALDKDSSPKDDVWDSVSII |
| | variant 1 | SFPEKEQEDVLQTL (SEQ ID NO: 230) |
| | NM_001329128_1 | |
| S086 | IFNLR1 | KTLMGNPWFQRAKMPRALDFSGHTHPVATFQPSRPESVNDLFLCPQKELTRG |
| | NM_170743_3 | VRPTPRVRAPATQQTRWKKDLAEDEEEEDEEDTEDGVSFQPYIEPPSFLGQEH |
| | | QAPGHSEAGGVDSGRPRAPLVPSEGSSAWDSSDRSWASTVDSSWDRAGSSGY |
| | | LAEKGPGQGPGGDGHQESLPPPEFSKDSGFLEELPEDNLSSWATWGTLPPEPN |
| | | LVPGGPPVSLQTLTFCWESSPEEEEEARESEIEDSDAGSWGAESTQRTEDRGRT |
| | | LGHYMAR (SEQ ID NO: 231) |
| S087 | IFNLR1 transcript | KTLMGNPWFQRAKMPRALELTRGVRPTPRVRAPATQQTRWKKDLAEDEEEE |
| | variant 2 | DEEDTEDGVSFQPYIEPPSFLGQEHQAPGHSEAGGVDSGRPRAPLVPSEGSSA |
| | NM_173064_2 | WDSSDRSWASTVDSSWDRAGSSGYLAEKGPGQGPGGDGHQESLPPPEFSKDS |
| | | GFLEELPEDNLSSWATWGTLPPEPNLVPGGPPVSLQTLTFCWESSPEEEEEARE |
| | | SEIEDSDAGSWGAESTQRTEDRGRTLGHYMAR (SEQ ID NO: 232) |
| S098 | IL1R1 transcript | KIDIVLWYRDSCYDFLPIKVLPEVLEKQCGYKLFIYGRDDYVGEDIVEVINENV |
| | variant 2 | KKSRRLIIILVRETSGFSWLGGSSEEQIAMYNALVQDGIKVVLLELEKIQDYEK |
| | NM_001288706_1 | MPESIKFIKQKHGAIRWSGDFTQGPQSAKTRFWKNVRYHMPVQRRSPSSKHQ |
| | | LLSPATKEKLQREAHVPLG (SEQ ID NO: 233) |
| S099 | IL1R1 transcript | KIDIVLWYRDSCYDFLPIKASDGKTYDAYILYPKTVGEGSTSDCDIFVFKVLPE |
| | variant 3 | VLEKQCGYKLFIYGRDDYVGEDIVEVINENVKKSRRLIIILVRETSGFSWLGGS |
| | NM_001320978_1 | SEEQIAMYNALVQDGIKVVLLELEKIQDYEKMPESIKFIKQKHGAIRWSGDFT |
| | | QGPQSAKTRFWKNVRYHMPVQRRSPSSKHQLLSPATKEKLQREAHVPLG |
| | | (SEQ ID NO: 234) |
| S100 | IL1RAP transcript | YRAHFGTDETILDGKEYDIYVSYARNAEEEEFVLLTLRGVLENEFGYKLCIFD |
| | variant 1 | RDSLPGGIVTDETLSFIQKSRRLLVVLSPNYVLQGTQALLELKAGLENMASRG |
| | NM_002182_3 | NINVILVQYKAVKETKVKELKRAKTVLTVIKWKGEKSKYPQGRFWKQLQVA |
| | | MPVKKSPRRSSSDEQGLSYSSLKNV (SEQ ID NO: 235) |
| S101 | IL1RAP transcript | YRAHFGTDETILDGKEYDIYVSYARNAEEEEFVLLTLRGVLENEFGYKLCIFD |
| | variant 6 | RDSLPGGNTVEAVFDFIQRSRRMIVVLSPDYVTEKSISMLEFKLGVMCQNSIAT |
| | NM_001167931_1 | KLIVVEYRPLEHPHPGILQLKESVSFVSWKGEKSKHSGSKFWKALRLALPLRS |
| | | LSASSGWNESCSSQSDISLDHVQRRRSRLKEPPELQSSERAAGSPPAPGTMSKH |
| | | RGKSSATCRCCVTYCEGENHLRNKSRAEIHNQPQWETHLCKPVPQESETQWI |
| | | QNGTRLEPPAPQISALALHHFTDLSNNNDFYIL (SEQ ID NO: 236) |
| S102 | IL1RL 1 transcript | LKMFWIEATLLWRDIAKPYKTRNDGKLYDAYVVYPRNYKSSTDGASRVEHF |
| | variant 1 | VHQILPDVLENKCGYTLCIYGRDMLPGEDVVTAVETNIRKSRRHIFILTPQITH |
| | NM_016232.4 | NKEFAYEQEVALHCALIQNDAKVILIEMEALSELDMLQAEALQDSLQHLMKV |
| | | QGTIKWREDHIANKRSLNSKFWKHVRYQMPVPSKIPRKASSLTPLAAQKQ |
| | | (SEQ ID NO: 237) |
| S103 | IL1RL2 | NIFKIDIVLWYRSAFHSTETIVDGKLYDAYVLYPKPHKESQRHAVDALVLNILP |
| | NM_003854.2 | EVLERQCGYKLFIFGRDEFPGQAVANVIDENVKLCRRLIVIVVPESLGFGLLKN |
| | | LSEEQIAVYSALIQDGMKVILIELEKIEDYTVMPESIQYIKQKHGAIRWHGDFT |
| | | EQSQCMKTKFWKTVRYHMPPRRCRPFPPVQLLQHTPCYRTAGPELGSRRKKC |
| | | TLTTG (SEQ ID NO: 238) |
| S104 | IL2RA transcript | TWQRRQRKSRRTI (SEQ ID NO: 239) |
| | variant 1 | |
| | NM_000417_2 | |
| S105 | IL2RB transcript | NCRNTGPWLKKVLKCNTPDPSKFFSQLSSEHGGDVQKWLSSPFPSSSFSPGGL |
| | variant 1 | APEISPLEVLERDKVTQLLLQQDKVPEPASLSSNHSLTSCFTNQGYFFFHLPDA |
| | NM_000878_4 | LEIEACQVYFTYDPYSEEDPDEGVAGAPTGSSPQPLQPLSGEDDAYCTFPSRDD |
| | | LLLFSPSLLGGPSPPSTAPGGSGAGEERMPPSLQERVPRDWDPQPLGPPTPGVP |
| | | DLVDFQPPPELVLREAGEEVPDAGPREGVSFPWSRPPGQGEFRALNARLPLNT |
| | | DAYLSLQELQGQDPTHLV (SEQ ID NO: 240) |
| S106 | IL2RG | ERTMPRIPTLKNLEDLVTEYHGNFSAWSGVSKGLAESLQPDYSERLCLVSEIPP |
| | NM_000206_2 | KGGALGEGPGASPCNQHSPYWAPPCYTLKPET (SEQ ID NO: 241) |
| S109 | IL3RA transcript | RRYLVMQRLFPRIPHMKDPIGDSFQNDKLVVWEAGKAGLEECLVTEVQVVQ |
| | variant 1 and 2 | KT (SEQ ID NO: 242) |
| | NM_002183_3 | |
| S110 | IL4R transcript | KIKKEWWDQIPNPARSRLVAIIIQDAQGSQWEKRSRGQEPAKCPHWKNCLTK |
| | variant 1 | LLPCFLEHNMKRDEDPHKAAKEMPFQGSGKSAWCPVEISKTVLWPESISVVR |
| | NM_000418_3 | CVELFEAPVECEEEEEVEEEKGSFCASPESSRDDFQEGREGIVARLTESLFLDLL |
| | | GEENGGFCQQDMGESCLLPPSGSTSAHMPWDEFPSAGPKEAPPWGKEQPLHL |
| | | EPSPPASPTQSPDNLTCTETPLVIAGNPAYRSFSNSLSQSPCPRELGPDPLLARH |
| | | LEEVEPEMPCVPQLSEPTTVPQPEPETWEQILRRNVLQHGAAAAPVSAPTSGY |
| | | QEFVHAVEQGGTQASAVVGLGPPGEAGYKAFSSLLASSAVSPEKCGFGASSG |
| | | EEGYKPFQDLIPGCPGDPAPVPVPLFTFGLDREPPRSPQSSHLPSSSPEHLGLEP |
| | | GEKVEDMPKPPLPQEQATDPLVDSLGSGIVYSALTCHLCGHLKQCHGQEDGG |
| | | QTPVMASPCCGCCCGDRSSPPTTPLRAPDPSPGGVPLEASLCPASLAPSGISEKS |
| | | KSSSSFHPAPGNAQSSSQTPKIVNFVSVGPTYMRVS (SEQ ID NO: 243) |
| S113 | IL4R transcript | KIKKEWWDQIPNPARSRLVAIIIQDAQGSQWEKRSRGQEPAKCPHWKNCLTK |
| | variant 1 | LLPCFLEHNMKRDEDPHKAAKEMPFQGSGKSAWCPVEISKTVLWPESISVVR |
| | NM_000418_3 | CVELFEAPVECEEEEEVEEEKGSFCASPESSRDDFQEGREGIVARLTESLFLDLL |
| | | GEENGGFCQQDMGESCLLPPSGSTSAHMPWDEFPSAGPKEAPPWGKEQPLHL |
| | | EPSPPASPTQSPDNLTCTETPLVIAGNPAYRSFSNSLSQSPCPRELGPDPLLARH |
| | | LEEVEPEMPCVPQLSEPTTVPQPEPETWEQILRRNVLQHGAAAAPVSAPTSGY |
| | | QEFVHAVEQGGTQASAVVGLGPPGEAGYKAFSSLLASSAVSPEKCGFGASSG |
| | | EEGYKPFQDLIPGCPGDPAPVPVPLFTFGLDREPPRSPQSSHLPSSSPEHLGLEP |
| | | GEKVEDMPKPPLPQEQATDPLVDSLGSGIVFSALTCHLCGHLKQCHGQEDGG |
| | | QTPVMASPCCGCCCGDRSSPPTTPLRAPDPSPGGVPLEASLCPASLAPSGISEKS |
| | | KSSSSFHPAPGNAQSSSQTPKIVNFVSVGPTYMRVS (SEQ ID NO: 244) |
| S115 | IL5RA transcript | KICHLWIKLFPPIPAPKSNIKDLFVTTNYEKAGSSETEIEVICYIEKPGVETLEDS |
| | variant 1 | VF (SEQ ID NO: 245) |
| | NM_000564_4 | |
| S116 | IL6R transcript | RFKKTWKLRALKEGKTSMHPPYSLGQLVPERPRPTPVLVPLISPPVSPSSLGSD |
| | variant 1 | NTSSHNRPDARDPRSPYDISNTDYFFPR (SEQ ID NO: 246) |
| | NM_000565_3 | |
| S117 | IL6ST transcript | NKRDLIKKHIWPNVPDPSKSHIAQWSPHTPPRHNFNSKDQMYSDGNFTDVSV |
| | variant 1 and 3 | VEIEANDKKPFPEDLKSLDLFKKEKINTEGHSSGIGGSSCMSSSRPSISSSDENES |
| | NM_002184_3 | SQNTSSTVQYSTVVHSGYRHQVPSVQVFSRSESTQPLLDSEERPEDLQLVDHV |
| | | DGGDGILPRQQYFKQNCSQHESSPDISHFERSKQVSSVNEEDFVRLKQQISDHI |
| | | SQSCGSGQMKMFQEVSAADAFGPGTEGQVERFETVGMEAATDEGMPKSYLP |
| | | QTVRQGGYMPQ (SEQ ID NO: 247) |
| S120 | IL7RA Isoform 1 | WKKRIKPIVWPSLPDHKKTLEHLCKKPRKNLNVSFNPESFLDCQIHRVDDIQA |
| | NM_002185.4 | RDEVEGFLQDTFPQQLEESEKQRLGGDVQSPNCPSEDVVITPESFGRDSSLTCL |
| | | AGNVSACDAPILSSSRSLDCRESGKNGPHVYQDLLLSLGTTNSTLPPPFSLQSGI |
| | | LTLNPVAQGQPILTSLGSNQEEAYVTMSSFYQNQ (SEQ ID NO: 248) |
| S121 | IL7RA Isoform 3 (C- | WKKRIKPIVWPSLPDHKKTLEHLCKKPRKVSVFGA (SEQ ID NO: 249) |
| | term deletion) | |
| | (interleukin 7 | |
| | receptor) | |
| S126 | IL9R transcript | KLSPRVKRIFYQNVPSPAMFFQPLYSVHNGNFQTWMGAHGAGVLLSQDCAG |
| | variant 1 | TPQGALEPCVQEATALLTCGPARPWKSVALEEEQEGPGTRLPGNLSSEDVLPA |
| | NM_002186_2 | GCTEWRVQTLAYLPQEDWAPTSLTRPAPPDSEGSRSSSSSSSSNNNNYCALGC |
| | | YGGWHLSALPGNTQSSGPIPALACGLSCDHQGLETQQGVAWVLAGHCQRPG |
| | | LHEDLQGMLLPSVLSKARSWTF (SEQ ID NO: 250) |
| S129 | IL10RA transcript | QLYVRRRKKLPSVLLFKKPSPFIFISQRPSPETQDTIHPLDEEAFLKVSPELKNL |
| | variant 1 | DLHGSTDSGFGSTKPSLQTEEPQFLLPDPHPQADRTLGNREPPVLGDSCSSGSS |
| | NM_001558_3 | NSTDSGICLQEPSLSPSTGPTWEQQVGSNSRGQDDSGIDLVQNSEGRAGDTQG |
| | | GSALGHHSPPEPEVPGEEDPAAVAFQGYLRQTRCAEEKATKTGCLEEESPLTD |
| | | GLGPKFGRCLVDEAGLHPPALAKGYLKQDPLEMTLASSGAPTGQWNQPTEE |
| | | WSLLALSSCSDLGISDWSFAHDLAPLGCVAAPGGLLGSFNSDLVTLPLISSLOS |
| | | SE (SEQ ID NO: 251) |
| S130 | IL10RB | ALLWCVYKKTKYAFSPRNSLPQHLKEFLGHPHHNTLLFFSFPLSDENDVFDKL |
| | NM_000628_4 | SVIAEDSESGKQNPGDSCSLGTPPGQGPQS (SEQ ID NO: 252) |
| S135 | IL11RA | RLRRGGKDGSPKPGFLASVIPVDRRPGAPNL (SEQ ID NO: 253) |
| | NM_001142784_2 | |
| S136 | IL12RB1 transcript | NRAARHLCPPLPTPCASSAIEFPGGKETWQWINPVDFQEEASLQEALVVEMSW |
| | variant 1 and 4 | DKGERTEPLEKTELPEGAPELALDTELSLEDGDRCKAKM (SEQ ID NO: 254) |
| | NM_005535_2 | |
| S137 | IL12RB1 transcript | NRAARHLCPPLPTPCASSAIEFPGGKETWQWINPVDFQEEASLQEALVVEMSW |
| | variant 3 | DKGERTEPLEKTELPEGAPELALDTELSLEDGDRCDR (SEQ ID NO: 255) |
| | NM_001290023_1 | |
| S138 | IL12RB2 transcript | HYFQQKVFVLLAALRPQWCSREIPDPANSTCAKKYPIAEEKTQLPLDRLLIDW |
| | variant 1 and 3 | PTPEDPEPLVISEVLHQVTPVFRHPPCSNWPQREKGIQGHQASEKDMMHSASS |
| | NM_001559_2 | PPPPRALQAESRQLVDLYKVLESRGSDPKPENPACPWTVLPAGDLPTHDGYLP |
| | | SNIDDLPSHEAPLADSLEELEPQHISLSVFPSSSLHPLTFSCGDKLTLDQLKMRC |
| | | DSLML (SEQ ID NO: 256) |
| S141 | IL13RA1 | KRLKIIIFPPIPDPGKIFKEMFGDQNDDTLHWKKYDIYEKQTKEETDSVVLIENL |
| | NM_001560_2 | KKASQ (SEQ ID NO: 257) |
| S142 | IL13RA2 | RKPNTYPKMIPEFFCDT (SEQ ID NO: 258) |
| | NM_000640_2 | |
| S143 | IL15RA transcript | KSRQTPPLASVEMEAMEALPVTWGTSSRDEDLENCSHHL (SEQ ID NO: 259) |
| | variant 4 | |
| | NM_001256765_1 | |
| S144 | IL17RA | CMTWRLAGPGSEKYSDDTKYTDGLPAADLIPPPLKPRKVWIIYSADHPLYVD |
| | NM_014339_6 | VVLKFAQFLLTACGTEVALDLLEEQAISEAGVMTWVGRQKQEMVESNSKIIV |
| | | LCSRGTRAKWQALLGRGAPVRLRCDHGKPVGDLFTAAMNMILPDFKRPACF |
| | | GTYVVCYFSEVSCDGDVPDLFGAAPRYPLMDRFEEVYFRIQDLEMFQPGRMH |
| | | RVGELSGDNYLRSPGGRQLRAALDRFRDWQVRCPDWFECENLYSADDQDAP |
| | | SLDEEVFEEPLLPPGTGIVKRAPLVREPGSQACLAIDPLVGEEGGAAVAKLEPH |
| | | LQPRGQPAPQPLHTLVLAAEEGALVAAVEPGPLADGAAVRLALAGEGEACPL |
| | | LGSPGAGRNSVLFLPVDPEDSPLGSSTPMASPDLLPEDVREHLEGLMLSLFEQS |
| | | LSCQAQGGCSRPAMVLTDPHTPYEEEQRQSVQSDQGYISRSSPQPPEGLTEME |
| | | EEEEEEQDPGKPALPLSPEDLESLRSLQRQLLFRQLQKNSGWDTMGSESEGPS |
| | | A (SEQ ID NO: 260) |
| S145 | IL17RB | RHERIKKTSFSTTTLLPPIKVLVVYPSEICFHHTICYFTEFLQNHCRSEVILEKWQ |
| | NM_018725_3 | KKKIAEMGPVQWLATQKKAADKVVFLLSNDVNSVCDGTCGKSEGSPSENSQ |
| | | DLFPLAFNLFCSDLRSQIHLHKYVVVYFREIDTKDDYNALSVCPKYHLMKDA |
| | | TAFCAELLHVKQQVSAGKRSQACHDGCCSL (SEQ ID NO: 261) |
| S146 | IL17RC transcript | KKDHAKGWLRLLKQDVRSGAAARGRAALLLYSADDSGFERLVGALASALCQ |
| | variant 1 | LPLRVAVDLWSRRELSAQGPVAWFHAQRRQTLQEGGVVVLLFSPGAVALCS |
| | NM_153460_3 | EWLQDGVSGPGAHGPHDAFRASLSCVLPDFLQGRAPGSYVGACFDRLLHPDA |
| | | VPALFRTVPVFTLPSQLPDFLGALQQPRAPRSGRLQERAEQVSRALQPALDSY |
| | | FHPPGTPAPGRGVGPGAGPGAGDGT (SEQ ID NO: 262) |
| S147 | IL17RC transcript | KKDHAKAAARGRAALLLYSADDSGFERLVGALASALCQLPLRVAVDLWSRR |
| | variant 4 | ELSAQGPVAWFHAQRRQTLQEGGVVVLLFSPGAVALCSEWLQDGVSGPGAH |
| | NM_001203263_1 | GPHDAFRASLSCVLPDFLQGRAPGSYVGACFDRLLHPDAVPALFRTVPVFTLP |
| | | SQLPDFLGALQQPRAPRSGRLQERAEQVSRALQPALDSYFHPPGTPAPGRGVG |
| | | PGAGPGAGDGT (SEQ ID NO: 263) |
| S148 | IL17RD transcript | CRKKQQENIYSHLDEESSESSTYTAALPRERLRPRPKVFLCYSSKDGQNHMNV |
| | variant 2 | VQCFAYFLQDFCGCEVALDLWEDFSLCREGQREWVIQKIHESQFIIVVCSKGM |
| | NM_017563_4 | KYFVDKKNYKHKGGGRGSGKGELFLVAVSAIAEKLRQAKQSSSAALSKFIAV |
| | | YFDYSCEGDVPGILDLSTKYRLMDNLPQLCSHLHSRDHGLQEPGQHTRQGSR |
| | | RNYFRSKSGRSLYVAICNMHQFIDEEPDWFEKQFVPFHPPPLRYREPVLEKFDS |
| | | GLVLNDVMCKPGPESDFCLKVEAAVLGATGPADSQHESQHGGLDQDGEARP |
| | | ALDGSAALQPLLHTVKAGSPSDMPRDSGIYDSSVPSSELSLPLMEGLSTDQTET |
| | | SSLTESVSSSSGLGEEEPPALPSKLLSSGSCKADLGCRSYTDELHAVAPL (SEQ |
| | | ID NO: 264) |
| S149 | IL17RE transcript | TCRRPQSGPGPARPVLLLHAADSEAQRRLVGALAELLRAALGGGRDVIVDLW |
| | variant 1 | EGRHVARVGPLPWLWAARTRVAREQGTVLLLWSGADLRPVSGPDPRAAPLL |
| | NM_153480_1 | ALLHAAPRPLLLLAYFSRLCAKGDIPPPLRALPRYRLLRDLPRLLRALDARPFA |
| | | EATSWGRLGARQRRQSRLELCSRLEREAARLADLG (SEQ ID NO: 265) |
| S154 | IL18R1 transcript | YRVDLVLFYRHLTRRDETLTDGKTYDAFVSYLKECRPENGEEHTFAVEILPRV |
| | variant 1 | LEKHFGYKLCIFERDVVPGGAVVDEIHSLIEKSRRLIIVLSKSYMSNEVRYELES |
| | NM_003855_3 | GLHEALVERKIKIILIEFTPVTDFTFLPQSLKLLKSHRVLKWKADKSLSYNSRF |
| | | WKNLLYLMPAKTVKPGRDEPEVLPVLSES (SEQ ID NO: 266) |
| S155 | IL18RAP | SALLYRHWIEIVLLYRTYQSKDQTLGDKKDFDAFVSYAKWSSFPSEATSSLSE |
| | NM_003853_3 | EHLALSLFPDVLENKYGYSLCLLERDVAPGGVYAEDIVSIIKRSRRGIFILSPNY |
| | | VNGPSIFELQAAVNLALDDQTLKLILIKFCYFQEPESLPHLVKKALRVLPTVTW |
| | | RGLKSVPPNSRFWAKMRYHMPVKNSQGFTWNQLRITSRIFQWKGLSRTETTG |
| | | RSSQPKEW (SEQ ID NO: 267) |
| S156 | IL20RA transcript | SIYRYIHVGKEKHPANLILIYGNEFDKRFFVPAEKIVINFITLNISDDSKISHQDM |
| | variant 1 | SLLGKSSDVSSLNDPQPSGNLRPPQEEEEVKHLGYASHLMEIFCDSEENTEGTS |
| | NM_014432_3 | LTQQESLSRTIPPDKTVIEYEYDVRTTDICAGPEEQELSLQEEVSTQGTLLESQA |
| | | ALAVLGPQTLQYSYTPQLQDLDPLAQEHTDSEEGPEEEPSTTLVDWDPQTGRL |
| | | CIPSLSSFDQDSEGCEPSEGDGLGEEGLLSRLYEEPAPDRPPGENETYLMQFME |
| | | EWGLYVQMEN (SEQ ID NO: 268) |
| S157 | IL20RB | WKMGRLLQYSCCPVVVLPDTLKITNSPQKLISCRREEVDACATAVMSPEELLR |
| | NM_144717_3 | AWIS (SEQ ID NO: 269) |
| S158 | IL21R transcript | SLKTHPLWRLWKKIWAVPSPERFFMPLYKGCSGDFKKWVGAPFTGSSLELGP |
| | variant 2 | WSPEVPSTLEVYSCHPPRSPAKRLQLTELQEPAELVESDGVPKPSFWPTAQNS |
| | NM_181078_2 | GGSAYSEERDRPYGLVSIDTVTVLDAEGPCTWPCSCEDDGYPALDLDAGLEPS |
| | | PGLEDPLLDAGTTVLSCGCVSAGSPGLGGPLGSLLDRLKPPLADGEDWAGGL |
| | | PWGGRSPGGVSESEAGSPLAGLDMDTFDSGFVGSDCSSPVECDFTSPGDEGPP |
| | | RSYLRQWVVIPPPLSSPGPQAS (SEQ ID NO: 270) |
| S161 | IL22RA1 | SYRYVTKPPAPPNSLNVQRVLTFQPLRFIQEHVLIPVFDLSGPSSLAQPVQYSQI |
| | NM_021258_3 | RVSGPREPAGAPQRHSLSEITYLGQPDISILQPSNVPPPQILSPLSYAPNAAPEVG |
| | | PPSYAPQVTPEAQFPFYAPQAISKVQPSSYAPQATPDSWPPSYGVCMEGSGKD |
| | | SPTGTLSSPKHLRPKGQLQKEPPAGSCMLGGLSLQEVTSLAMEESQEAKSLHQ |
| | | PLGICTDRTSDPNVLHSGEEGTPQYLKGQLPLLSSVQIEGHPMSLPLQPPSRPCS |
| | | PSDQGPSPWGLLESLVCPKDEAKSPAPETSDLEQPTELDSLFRGLALTVQWES |
| | | (SEQ ID NO: 271) |
| S165 | IL23R | NRSFRTGIKRRILLLIPKWLYEDIPNMKNSNVVKMLQENSELMNNNSSEQVLY |
| | NM_144701_2 | VDPMITEIKEIFIPEHKPTDYKKENTGPLETRDYPQNSLFDNTTVVYIPDLNTGY |
| | | KPQISNFLPEGSHLSNNNEITSLTLKPPVDSLDSGNNPRLQKHPNFAFSVSSVNS |
| | | LSNTIFLGELSLILNQGECSSPDIQNSVEEETTMLLENDSPSETIPEQTLLPDEFV |
| | | SCLGIVNEELPSINTYFPQNILESHENRISLLEK (SEQ ID NO: 272) |
| S168 | IL27RA | TSGRCYHLRHKVLPRWVWEKVPDPANSSSGQPHMEQVPEAQPLGDLPILEVE |
| | NM_004843_3 | EMEPPPVMESSQPAQATAPLDSGYEKHFLPTPEELGLLGPPRPQVLA (SEQ ID |
| | | NO: 273) |
| S169 | IL27RA | TSWVWEKVPDPANSSSGQPHMEQVPEAQPLGDLPILEVEEMEPPPVMESSQP |
| | NM_004843_3 | AQATAPLDSGYEKHFLPTPEELGLLGPPRPQVLA (SEQ ID NO: 274) |
| S170 | IL31RA transcript | KKPNKLTHLCWPTVPNPAESSIATWHGDDFKDKLNLKESDDSVNTEDRILKPC |
| | variant 1 | STPSDKLVIDKLVVNFGNVLQEIFTDEARTGQENNLGGEKNGYVTCPFRPDCP |
| | NM_139017_5 | LGKSFEELPVSPEIPPRKSQYLRSRMPEGTRPEAKEQLLFSGQSLVPDHLCEEG |
| | | APNPYLKNSVTAREFLVSEKLPEHTKGEV (SEQ ID NO: 275) |
| S171 | IL31RA transcript | KKPNKLTHLCWPTVPNPAESSIATWHGDDFKDKLNLKESDDSVNTEDRILKPC |
| | variant 4 | STPSDKLVIDKLVVNFGNVLQEIFTDEARTGQENNLGGEKNGTRILSSCPTSI |
| | NM_001242638_1 | (SEQ ID NO: 276) |
| S174 | LEPR transcript | SHQRMKKLFWEDVPNPKNCSWAQGLNFQKPETFEHLFIKHTASVTCGPLLLE |
| | variant 1 | PETISEDISVDTSWKNKDEMMPTTVVSLLSTTDLEKGSVCISDQFNSVNFSEAE |
| | NM_002303_5 | GTEVTYEDESQRQPFVKYATLISNSKPSETGEEQGLINSSVTKCFSSKNSPLKDS |
| | | FSNSSWEIEAQAFFILSDQHPNIISPHLTFSEGLDELLKLEGNFPEENNDKKSIYY |
| | | LGVTSIKKRESGVLLTDKSRVSCPFPAPCLFTDIRVLQDSCSHFVENNINLGTSS |
| | | KKTFASYMPQFQTCSTQTHKIMENKMCDLTV (SEQ ID NO: 277) |
| S175 | LEPR transcript | SHQRMKKLFWEDVPNPKNCSWAQGLNFQKMLEGSMFVKSHHHSLISSTQGH |
| | variant 2 | KHCGRPQGPLHRKTRDLCSLVYLLTLPPLLSYDPAKSPSVRNTQE (SEQ ID |
| | NM_001003680_3 | NO: 278) |
| S176 | LEPR transcript | SHQRMKKLFWEDVPNPKNCSWAQGLNFQKRTDIL (SEQ ID NO: 279) |
| | variant 3 | |
| | NM_001003679_3 | |
| S177 | LEPR transcript | SHQRMKKLFWEDVPNPKNCSWAQGLNFQKKMPGTKELLGGGWLT (SEQ ID |
| | variant 5 | NO: 280) |
| | NM_001198688_1 | |
| S180 | LIFR | YRKREWIKETFYPDIPNPENCKALQFQKSVCEGSSALKTLEMNPCTPNNVEVL |
| | NM_001127671_1 | ETRSAFPKIEDTEIISPVAERPEDRSDAEPENHVVVSYCPPIIEEEIPNPAADEAG |
| | | GTAQVIYIDVQSMYQPQAKPEEEQENDPVGGAGYKPQMHLPINSTVEDIAAEE |
| | | DLDKTAGYRPQANVNTWNLVSPDSPRSIDSNSEIVSFGSPCSINSRQFLIPPKDE |
| | | DSPKSNGGGWSFTNFFQNKPND (SEQ ID NO: 281) |
| S183 | LMP1 NC_007605_1 | YYHGQRHSDEHHHDDSLPHPQQATDDSGHESDSNSNEGRHHLLVSGAGDGP |
| | | PLCSQNLGAPGGGPDNGPQDPDNTDDNGPQDPDNTDDNGPHDPLPQDPDNT |
| | | DDNGPQDPDNTDDNGPHDPLPHSPSDSAGNDGGPPQLTEEVENKGGDQGPPL |
| | | MTDGGGGHSHDSGHGGGDPHLPTLLLGSSGSGGDDDDPHGPVQLSYYD |
| | | (SEQ ID NO: 282) |
| S186 | MPL NM_005373_2 | RWQFPAHYRRLRHALWPSLPDLHRVLGQYLRDTAALSPPKATVSDTCEEVEP |
| | | SLLEILPKSSERTPLPLCSSQAQMDYRRLQPSCLGTMPLSVCPPMAESGSCCTT |
| | | HIANHSYLPLSYWQQP (SEQ ID NO: 283) |
| S189 | MYD88 transcript | MAAGGPGAGSAAPVSSTSSLPLAALNMRVRRRLSLFLNVRTQVAADWTALA |
| | variant 1 | EEMDFEYLEIRQLETQADPTGRLLDAWQGRPGASVGRLLELLTKLGRDDVLL |
| | NM_001172567_1 | ELGPSIEEDCQKYILKQQQEEAEKPLQVAAVDSSVPRTAELAGITTLDDPLGH |
| | | MPERFDAFICYCPSDIQFVQEMIRQLEQTNYRLKLCVSDRDVLPGTCVWSIASE |
| | | LIEKRLARRPRGGCRRMVVVVSDDYLQSKECDFQTKFALSLSPGAHQKRLIPI |
| | | KYKAMKKEFPSILRFITVCDYTNPCTKSWFWTRLAKALSLP (SEQ ID NO: 284) |
| S190 | MYD88 transcript | MAAGGPGAGSAAPVSSTSSLPLAALNMRVRRRLSLFLNVRTQVAADWTALA |
| | variant 2 | EEMDFEYLEIRQLETQADPTGRLLDAWQGRPGASVGRLLELLTKLGRDDVLL |
| | NM_002468_4 | ELGPSIEEDCQKYILKQQQEEAEKPLQVAAVDSSVPRTAELAGITTLDDPLGH |
| | | MPERFDAFICYCPSDIQFVQEMIRQLEQTNYRLKLCVSDRDVLPGTCVWSIASE |
| | | LIEKRCRRMVVVVSDDYLQSKECDFQTKFALSLSPGAHQKRLIPIKYKAMKKE |
| | | FPSILRFITVCDYTNPCTKSWFWTRLAKALSLP (SEQ ID NO: 285) |
| S191 | MYD88 transcript | MAAGGPGAGSAAPVSSTSSLPLAALNMRVRRRLSLFLNVRTQVAADWTALA |
| | variant 3 | EEMDFEYLEIRQLETQADPTGRLLDAWQGRPGASVGRLLELLTKLGRDDVLL |
| | NM_001172568_1 | ELGPSIGHMPERFDAFICYCPSDIQFVQEMIRQLEQTNYRLKLCVSDRDVLPGT |
| | | CVWSIASELIEKRCRRMVVVVSDDYLQSKECDFQTKFALSLSPGAHQKRLIPIK |
| | | YKAMKKEFPSILRFITVCDYTNPCTKSWFWTRLAKALSLP (SEQ ID NO: 286) |
| S192 | MYD88 transcript | MAAGGPGAGSAAPVSSTSSLPLAALNMRVRRRLSLFLNVRTQVAADWTALA |
| | variant 4 | EEMDFEYLEIRQLETQADPTGRLLDAWQGRPGASVGRLLELLTKLGRDDVLL |
| | NM_001172569_1 | ELGPSIEEDCQKYILKQQQEEAEKPLQVAAVDSSVPRTAELAGITTLDDPLGAA |
| | | GWWWLSLMITCRARNVTSRPNLHSASLQVPIRSD (SEQ ID NO: 287) |
| S193 | MYD88 transcript | MAAGGPGAGSAAPVSSTSSLPLAALNMRVRRRLSLFLNVRTQVAADWTALA |
| | variant 5 | EEMDFEYLEIRQLETQADPTGRLLDAWQGRPGASVGRLLELLTKLGRDDVLL |
| | NM_001172566_1 | ELGPSIGAAGWWWLSLMITCRARNVTSRPNLHSASLQVPIRSD (SEQ ID |
| | | NO: 288) |
| S194 | MYD88 transcript | MAAGGPGAGSAAPVSSTSSLPLAALNMRVRRRLSLFLNVRTQVAADWTALA |
| | variant 1 | EEMDFEYLEIRQLETQADPTGRLLDAWQGRPGASVGRLLELLTKLGRDDVLL |
| | NM_001172567_1 | ELGPSIEEDCQKYILKQQQEEAEKPLQVAAVDSSVPRTAELAGITTLDDPLGH |
| | | MPERFDAFICYCPSDI (SEQ ID NO: 289) |
| S195 | MYD88 transcript | MAAGGPGAGSAAPVSSTSSLPLAALNMRVRRRLSLFLNVRTQVAADWTALA |
| | variant 3 | EEMDFEYLEIRQLETQADPTGRLLDAWQGRPGASVGRLLELLTKLGRDDVLL |
| | NM_001172568_1 | ELGPSIGHMPERFDAFICYCPSDI (SEQ ID NO: 290) |
| S196 | MYD88 transcript | MAAGGPGAGSAAPVSSTSSLPLAALNMRVRRRLSLFLNVRTQVAADWTALA |
| | variant 1 | EEMDFEYLEIRQLETQADPTGRLLDAWQGRPGASVGRLLELLTKLGRDDVLL |
| | NM_001172567_1 | ELGPSIEEDCQKYILKQQQEEAEKPLQVAAVDSSVPRTAELAGITTLDDPLGH |
| | | MPERFDAFICYCPSDIQFVQEMIRQLEQTNYRLKLCVSDRDVLPGTCVWSIASE |
| | | LIEKRLARRPRGGCRRMVVVVSDDYLQSKECDFQTKFALSLSPGAHQKRPIPI |
| | | KYKAMKKEFPSILRFITVCDYTNPCTKSWFWTRLAKALSLP (SEQ ID NO: 291) |
| S197 | MYD88 transcript | MAAGGPGAGSAAPVSSTSSLPLAALNMRVRRRLSLFLNVRTQVAADWTALA |
| | variant 2 | EEMDFEYLEIRQLETQADPTGRLLDAWQGRPGASVGRLLELLTKLGRDDVLL |
| | NM_002468_4 | ELGPSIEEDCQKYILKQQQEEAEKPLQVAAVDSSVPRTAELAGITTLDDPLGH |
| | | MPERFDAFICYCPSDIQFVQEMIRQLEQTNYRLKLCVSDRDVLPGTCVWSIASE |
| | | LIEKRCRRMVVVVSDDYLQSKECDFQTKFALSLSPGAHQKRPIPIKYKAMKKE |
| | | FPSILRFITVCDYTNPCTKSWFWTRLAKALSLP (SEQ ID NO: 292) |
| S198 | MYD88 transcript | MAAGGPGAGSAAPVSSTSSLPLAALNMRVRRRLSLFLNVRTQVAADWTALA |
| | variant 3 | EEMDFEYLEIRQLETQADPTGRLLDAWQGRPGASVGRLLELLTKLGRDDVLL |
| | NM_001172568_1 | ELGPSIGHMPERFDAFICYCPSDIQFVQEMIRQLEQTNYRLKLCVSDRDVLPGT |
| | | CVWSIASELIEKRCRRMVVVVSDDYLQSKECDFQTKFALSLSPGAHQKRPIPIK |
| | | YKAMKKEFPSILRFITVCDYTNPCTKSWFWTRLAKALSLP (SEQ ID NO: 293) |
| S199 | OSMR transcript | KSQWIKETCYPDIPDPYKSSILSLIKFKENPHLIIMNVSDCIPDAIEVVSKPEGTKI |
| | variant 4 | QFLGTRKSLTETELTKPNYLYLLPTEKNHSGPGPCICFENLTYNQAASDSGSCG |
| | NM_001323505_1 | HVPVSPKAPSMLGLMTSPENVLKALEKNYMNSLGEIPAGETSLNYVSQLASP |
| | | MFGDKDSLPTNPVEAPHCSEYKMQMAVSLRLALPPPTENSSLSSITLLDPGEH |
| | | YC (SEQ ID NO: 294) |
| S202 | PRLR transcript | KGYSMVTCIFPPVPGPKIKGFDAHLLEKGKSEELLSALGCQDFPPTSDYEDLLV |
| | variant 1 | EYLEVDDSEDQHLMSVHSKEHPSQGMKPTYLDPDTDSGRGSCDSPSLLSEKC |
| | NM_000949_6 | EEPQANPSTFYDPEVIEKPENPETTHTWDPQCISMEGKIPYFHAGGSKCSTWPL |
| | | PQPSQHNPRSSYHNITDVCELAVGPAGAPATLLNEAGKDALKSSQTIKSREEG |
| | | KATQQREVESFHSETDQDTPWLLPQEKTPFGSAKPLDYVEIHKVNKDGALSLL |
| | | PKQRENSGKPKKPGTPENNKEYAKVSGVMDNNILVLVPDPHAKNVACFEESA |
| | | KEAPPSLEQNQAEKALANFTATSSKCRLQLGGLDYLDPACFTHSFH (SEQ ID |
| | | NO: 295) |
| S211 | TNFRSF4 | ALYLLRRDQRLPPDAHKPPGGGSFRTPIQEEQADAHSTLAKI (SEQ ID NO:296) |
| | NM_003327_3 | |
| S212 | TNFRSF8 transcript | HRRACRKRIRQKLHLCYPVQTSQPKLELVDSRPRRSSTQLRSGASVTEPVAEE |
| | variant 1 | RGLMSQPLMETCHSVGAAYLESLPLQDASPAGGPSSPRDLPEPRVSTEHTNNK |
| | NM_001243_4 | IEKIYIMKADTVIVGTVKAELPEGRGLAGPAEPELEEELEADHTPHYPEQETEP |
| | | PLGSCSDVMLSVEEEGKEDPLPTAASGK (SEQ ID NO: 297) |
| S213 | TNFRSF9 | KRGRKKLLYIFKQPFMRPVQTTQEEDGCCRFPEEEEGGCEL (SEQ ID |
| | NM_001561_5 | NO: 298) |
| S214 | TNFRSF14 transcript | CVKRRKPRGDVVKVIVSVQRKRQEAEGEATVIEALQAPPDVTTVAVEETIPSF |
| | variant 1 | TGRSPNH (SEQ ID NO: 299) |
| | NM_003820_3 | |
| S215 | TNFRSF18 transcript | QLGLHIWQLRSQCMWPRETQLLLEVPPSTEDARSCQFPEEERGERSAEEKGRL |
| | variant 1 | GDLWV (SEQ ID NO: 300) |
| | NM_004195_2 | |
| S216 | TNFRSF18 transcript | QLGLHIWQLRKTQLLLEVPPSTEDARSCQFPEEERGERSAEEKGRLGDLWV |
| | variant 3 | (SEQ ID NO: 301) |
| | NM_148902_1 | |
| X001 | Linker | GSGGSEGGGSEGGAATAGSGSGS (SEQ ID NO: 302) |

What is claimed is:

1. A replication incompetent recombinant retroviral particle (RIP), comprising a viral envelope, a nucleic acid encoding a packaging signal, Gag and a membrane-bound hyaluronidase.

2. A replication incompetent recombinant retroviral particle (RIP) formulation, comprising a plurality of RIPs, wherein each of the RIPs comprises a viral envelope, a nucleic acid encoding a packaging element, Gag, and a membrane-bound hyaluronidase polypeptide.

3. The RIP of claim 1 or the RIP formulation of claim 2, wherein the RIP further comprises a polynucleotide comprising one or more transcriptional units, wherein each of the one or more transcriptional units is operatively linked to a promoter active in a target cell.

4. The RIP of claim 1 or the RIP formulation of claim 2, wherein the hyaluronidase polypeptide comprises a PH20, Hyal-1, Hyal-2, Hyal-3, Hyal-4, or Hyal-5 hyaluronidase polypeptide.

5. The RIP of claim 1 or the RIP formulation of claim 2, wherein the hyaluronidase polypeptide comprises a PH20, Hyal-1, Hyal-2, Hyal-3, or Hyal-5 hyaluronidase polypeptide.

6. The RIP or RIP formulation of claim 4, wherein the hyaluronidase polypeptide comprises a human PH20, bovine PH20, human Hyal-1, human Hyal-2, human Hyal-3, human Hyal-4, or bovine Hyal-5 hyaluronidase polypeptide.

7. The RIP or RIP formulation of claim 4, wherein the hyaluronidase polypeptide comprises a human PH20, human Hyal-1, human Hyal-2, human Hyal-3, or bovine Hyal-5 hyaluronidase polypeptide.

8. The RIP or RIP formulation of claim 6, wherein the hyaluronidase polypeptide comprises a bovine Hyal-5 hyaluronidase polypeptide.

9. The RIP of claim 1 or the RIP formulation of claim 2, wherein the hyaluronidase polypeptide comprises an amino acid sequence with at least 90% sequence identity to any one of SEQ ID NOs:422-480 or 1040-1077.

10. The RIP of claim 1 or the RIP formulation of claim 2, wherein the hyaluronidase polypeptide comprises an amino acid sequence with at least 90% sequence identity to any one of SEQ ID NOs:422-460, 470-480, 1040-1067, or 1077.

11. The RIP of claim 1 or the RIP formulation of claim 2, wherein the hyaluronidase polypeptide comprises an amino acid sequence with at least 90% sequence identity to any one of SEQ ID NOs:422-451, 461-480, 1040-1060, or 1068-1077.

12. The RIP of claim 1 or the RIP formulation of claim 2, wherein the hyaluronidase polypeptide comprises an amino acid sequence with at least 90% sequence identity to any one of SEQ ID NOs:422-423, 434, 443, 452, 470-471, or 475-480.

13. The RIP of claim 1 or the RIP formulation of claim 2, wherein the hyaluronidase polypeptide is a chimeric hyaluronidase polypeptide.

14. The RIP of claim 1 or the RIP formulation of claim 2, wherein the hyaluronidase polypeptide comprises a first stretch of at least 20 amino acids with at least 90% sequence identity to a first hyaluronidase and a second stretch of at least 20 amino acids with at least 90% sequence identity to a second hyaluronidase.

15. The RIP of claim 1 or the RIP formulation of claim 2, wherein the hyaluronidase polypeptide comprises from amino terminus to carboxy terminus Region 1, Region 2, Region 3, Region 4, Region 5, and Region 6, and wherein one or more Regions are from a first hyaluronidase and one or more other Regions are from a second hyaluronidase.

16. The RIP of claim 1 or the RIP formulation of claim 2, wherein the hyaluronidase polypeptide comprises from amino terminus to carboxy terminus Region 1, Region 2, Region 3, Region 4, Region 5, and Region 6, and wherein one or more Regions are from a first hyaluronidase and one or more other Regions are from a second hyaluronidase.

17. The RIP of claim 1 or the RIP formulation of claim 2, wherein the hyaluronidase polypeptide comprises an amino acid sequence with at least 90% sequence identity to any one of SEQ ID NOs:544-1003.

18. The RIP of claim 1 or the RIP formulation of claim 2, wherein the hyaluronidase polypeptide comprises an amino acid sequence with at least 90% sequence identity to any one of SEQ ID NOs:544-1003.

19. The RIP of claim 1 or the RIP formulation of claim 2, wherein the hyaluronidase polypeptide comprises from amino terminus to carboxy terminus Region 1, Region 2, Region 3, Region 4, Region 5, and Region 6, and wherein Region 5 comprises an amino acid sequence with at least 90% sequence identity to any one of SEQ ID NOs:499, 509, 518, 528, or 538.

20. The RIP or RIP formulation of claim 19, wherein Region 1, Region 2, Region 3, Region 4, and Region 6 include amino acid sequences with at least 90% sequence identity to SEQ ID NOs:483, 485, 487, 488, and 490, respectively.

21. The RIP of claim 1 or the RIP formulation of claim 2, wherein the hyaluronidase polypeptide comprises an amino acid sequence with at least 90% sequence identity to any one of SEQ ID NOs:554-558.

22. The RIP of claim 1 or the RIP formulation of claim 2, wherein the hyaluronidase polypeptide comprises an amino acid sequence with at least 90% sequence identity to any one of SEQ ID NOs:555-557.

23. A delivery solution, comprising the RIP or RIP formulation of any one of claims 1 to 22.

24. A pharmaceutical composition, comprising the RIP or RIP formulation of any one of claims 1 to 22 or the delivery solution of claim 23.

25. A method of administering to a subject, comprising administering the RIP or RIP formulation of any one of claims 1 to 22, the delivery solution of claim 23, or the pharmaceutical composition of claim 24 to the subject.

26. A method of treating a subject, comprising administering the RIP or RIP formulation of any one of claims 1 to 22, the delivery solution of claim 23, or the pharmaceutical composition of claim 24 to the subject, thereby treating the subject.

27. Replication incompetent recombinant retroviral particles (RIPs) for use in administering a RIP formulation to a subject, wherein use of the RIPs comprises, administering the RIP formulation to the subject, wherein the RIP formulation comprises the RIPs, wherein the RIPs comprise a polynucleotide comprising one or more transcriptional units, wherein each of the one or more transcriptional units is operatively linked to a promoter active in a target cell, and wherein the RIPs comprise a membrane-bound hyaluronidase polypeptide.

28. Replication incompetent recombinant retroviral particles (RIPs) for use in modifying target cells in a subject, wherein use of the RIPs comprises administering to the subject, a RIP formulation comprising the RIPs, wherein the RIPs comprise a polynucleotide comprising one or more transcriptional units, wherein each of the one or more transcriptional units is operatively linked to a promoter active in a target cell, and
wherein the RIPs comprise a membrane-bound hyaluronidase polypeptide,
wherein said administering facilitates association of the target cells with the RIPs, wherein the target cells are present in the subject, and wherein the RIPs modify the target cells to form a population of modified target cells in the subject.

29. The RIP or RIP formulation of any one of claims 1 to 22, the delivery solution of claim 23, the pharmaceutical composition of claim 24, the method of any one of claims 25 or 26, or the RIPs of any one of claims 27 or 28, wherein the RIP or each of the RIPs comprises a targeting element on its surface.

30. The RIP, RIP formulation, delivery solution, pharmaceutical composition, method, or RIPs of claim 29, wherein the targeting element binds to 5T4, αvβ6 integrin, alpha-fetoprotein (AFP), an abnormal p53 protein, an abnormal ras protein, ASGPR, ASGPR 1, ASGPR1, Asialoglycoprotein Receptor 1 (ASGR1), Axl, B cell maturation antigen (BCMA), B7-H3, B7-H6, CA19-9, CAIX, calretinin, cancer antigen-125 (CA-125), carcinoembryonic antigen (CEA), CCR5, CCR7, CD104, CD105, CD117, CD11b, CD11c, CD123, CD133, CD138, CD14, CD140b, CD146, CD16, CD171, CD184, CD19, CD20, CD21, CD22, CD23, CD24, CD25, CD27, CD28, CD3, CD30, CD31, CD33, CD34, CD37, CD38, CD4, CD40, CD44, CD44v6, CD44v7/8, CD45, CD45RA, CD45RO, CD5, CD55 (DAF1), CD56, CD57, CD62L, CD7, CD70, CD8, CD86, CD90, CD99, chromogranin, CK18, c-kit, c-met, cone opsin, CS1, CS-1, CSPG4, C-Type Lectin Domain Family 4 Member H1 (CLEC4H1), CXCR4, DC-SIGN (also known as CD209), desmin, EGFR, EGFRvIII (epidermal growth factor variant III), EGP2, EGP40, Encephalopsin (OPN3), EpCAM, EphA2, Eph-B2, epithelial membrane protein (EMA), epithelial tumor antigen (ETA), FAP (Fibroblast Activation Protein), fetal AchR, FoxP3, FRα, galactin, GD2, GD2 (ganglioside G2), GD3, GPC3, GUCY2C, Hepatic Lectin H1 (HL-1), HER1, Her2 (ERBB2), high molecular weight-melanoma associated antigen (HMW-MAA), HLA-A1+MAGE1, HLA-A1+NY-ESO-1, HLA-DR, ICAM-1, IL11Rα, IL-11Rα, IL13Rα2, IL-13Rα2, integrin αvβ3 (CD61), integrins, Itm2a, kappa, KIRs, Kras, K-Ras (V-Ki-ras2 Kirsten rat sarcoma viral oncogene), Kras G12D, Krt8, LICAM, lambda, Lewis-Y, MAGE-A1, melanoma-associated antigen (MAGE), mesothelin, MET, motor neuron marker non-phosphorylated neurofilament-H (SMI-32), MUC1, MUC-1, Muc16, MUC16 ecto, muscle-specific actin (MSA), MyoD, myo-D1, myogenin, NCAM, neurofilament, neuron-specific enolase (NSE), New York esophageal squamous cell carcinoma antigen (NYESO1), NKG2D, NKG2D Ligands, NY-ESO-1, PAP (prostatic acid phosphatase), Pax7, PDL-1, placental alkaline phosphatase, PRAME, prostate stem cell antigen (PSCA), prostein, protein melan-A (melanoma antigen recognized by T lymphocytes; MART-1), PSCA, PSMA (prostate-specific membrane antigen), Ral-B, recoverin, rhodopsin, Rlbp1, ROR1, ROR2, Rrh, Silv (also known as Pmel17), sperm protein 17 (Sp17), STEAP1 (six-transmembrane epithelial antigen of the prostate 1), Survivin, synaptophysin, TAG72, TARP (T cell receptor gamma alternate reading frame protein), TCR, TEMs, the dimeric form of the pyruvate kinase isoenzyme type M2 (tumor M2-PK), thyroglobulin, thyroid transcription factor-1, Trp-p8, tyrosinase, Vap2, VEGFR2, or WT-1.

31. The RIP, RIP formulation, delivery solution, pharmaceutical composition, method, or RIPs of claim 29, wherein the targeting element binds to a cytokine, chemokine, ligand, or receptor.

32. The RIP, RIP formulation, delivery solution, pharmaceutical composition, method, or RIPs of claim 29, wherein the targeting element binds to CD3, CD4, CD8, or CCR5.

33. The RIP, RIP formulation, delivery solution, pharmaceutical composition, method, or RIPs of claim 29, wherein the targeting element is an activation element.

34. The RIP, RIP formulation, delivery solution, pharmaceutical composition, method, or RIPs of claim 29, wherein the targeting element is a binding element.

35. The RIP, RIP formulation, delivery solution, pharmaceutical composition, method, or RIPs of claim 29, wherein the targeting element is a pseudotyping element.

36. The RIP or RIP formulation of any one of claims 1 to 22, the delivery solution of claim 23, the pharmaceutical composition of claim 24, the method of any one of claims 25 or 26, or the RIPs of any one of claims 27 or 28, wherein the nucleic acid encodes a gene of interest (GOI) or is a GOI.

37. The RIP, RIP formulation, delivery solution, pharmaceutical composition, method, or RIPs of claim 36, wherein the GOI is present in one of the one or more transcriptional units.

38. The RIP, RIP formulation, delivery solution, pharmaceutical composition, method, or RIPs of claim 36, wherein the GOI comprises an engineered signaling polypeptide, a hyaluronidase polypeptide, a checkpoint-inhibiting ligand, a nucleic acid sequence or polypeptide that stimulates an immune response, a nucleic acid sequence or polypeptide that stimulates apoptosis, a polypeptide that functions as a transporter, a polypeptide that stimulates growth, a toxic protein, a nucleic acid sequence or polypeptide that stimulates autophagy and shutdown of protein synthesis, a nucleic acid sequence or polypeptide that stimulates induction of anti-tumoral immunity, a suicide gene, a safety switch, an anti-idiotype polypeptide, any polypeptide known to be deleted or defective in diseases, CRISPR-Cas, guide RNAs, and/or an inhibitory RNA.

39. The RIP, RIP formulation, delivery solution, pharmaceutical composition, method, or RIPs of claim 36, wherein the GOI comprises an engineered signaling polypeptide.

40. The RIP, RIP formulation, delivery solution, pharmaceutical composition, method, or RIPs of claim 39, wherein the engineered signaling polypeptide is a chimeric antigen receptor, TCR, or lymphoproliferative element.

41. The RIP, RIP formulation, delivery solution, pharmaceutical composition, method, or RIPs of claim 40, wherein the engineered signaling polypeptide is the chimeric antigen receptor.

42. The RIP, RIP formulation, delivery solution, pharmaceutical composition, method, or RIPs of claim 40, wherein the engineered signaling polypeptide is the TCR.

43. The RIP, RIP formulation, delivery solution, pharmaceutical composition, method, or RIPs of claim 40, wherein the engineered signaling polypeptide is the lymphoproliferative element.

44. The RIP, RIP formulation, delivery solution, pharmaceutical composition, method, or RIPs of claim 36, wherein the GOI is not a targeting element or a membrane-bound hyaluronidase.

45. The RIP, RIP formulation, delivery solution, pharmaceutical composition, method, or RIPs of claim 36, wherein the GOI is a hyaluronidase.

46. The RIP, RIP formulation, delivery solution, pharmaceutical composition, method, or RIPs of claim 45, wherein the hyaluronidase comprises an amino acid sequence with at least 90% sequence identity to any one of SEQ ID NOs:544-1003.
